(19) United States
(12) Patent Application Publication
(10) Pub. No.: US 2026/0286360 A1
(43) Pub. Date: Sep. 24, 2026

(54) COMPOSITIONS AND METHODS TO MODULATE IDIOPATHIC PULMONARY FIBROTIC ASSOCIATED LONG NONCODING RNAS

(71) Applicant: HAYA Therapeutics SA, Epalinges (CH)

(72) Inventors: Heather D. LYNN, San Diego, CA (US); Rudi MICHELETTI, San Diego, CA (US); Samir OUNZAIN, Lausanne (CH); Marco MINA, Epalinges (CH); Daniel Roman BLESSING, Saint-Sulpice (CH)

(21) Appl. No.: 19/575,786

(22) Filed: Mar. 23, 2026

Related U.S. Application Data
(63) Continuation of application No. PCT/IB2024/000524, filed on Sep. 24, 2024.
(60) Provisional application No. 63/540,328, filed on Sep. 25, 2023.
(60) Provisional application No. 63/616,204, filed on Dec. 29, 2023.
(60) Provisional application No. 63/648,458, filed on May 16, 2024.

(51) Int. Cl. C12N 15/113; C12Q 1/6883

(52) U.S. Cl. CPC C12N 15/113; C12Q 1/6883; C12N 2310/113; C12N 2310/315; C12N 2310/321; C12N 2310/3231; C12N 2310/341; C12N 2320/34; C12Q 2600/118; C12Q 2600/158

(57) ABSTRACT

Disclosed herein are modulators of a long non-coding RNA (lncRNA), wherein an expression of the lncRNA is associated with idiopathic pulmonary fibrosis. Further provided herein are methods of treating idiopathic pulmonary fibrosis a subject in need thereof. Also, provided herein are methods of predicting severity and progression of idiopathic pulmonary fibrosis based on expression of the lncRNAs described herein.

### CROSS REFERENCE

This application is a continuation of International Application No. PCT/IB2024/000524, filed Sep. 24, 2024, which claims the benefit of U.S. Provisional Application No. 63/540,328, filed Sep. 25, 2023, U.S. Provisional Application No. 63/616,204, filed Dec. 29, 2023, and U.S. Provisional Application No. 63/648,458, filed May 16, 2024, each of which is incorporated herein by reference in its entirety.

### SEQUENCE LISTING

This instant application contains a Sequence Listing which has been submitted electronically in XML format and is hereby incorporated by reference in its entirety. Said XML copy, created on Mar. 23, 2026, is named 60448-702-301_SL.xml and is 3,618,217 bytes in size.

### BACKGROUND

Only a small portion of mammalian genome is transcribed to protein-coding mRNAs, with the rest of genome transcribed into an RNA form is transcribed to non-coding RNAs. Among them, long non-coding RNA (lncRNA) is a type of RNA, usually more than 200 nucleotides in length, which is not translated into protein. LncRNAs have been shown to regulate gene expression networks at different levels via various mechanisms (see, e.g., Yao et al., Nature Cell Biology, 21, pages 542-551, 2019).

Idiopathic pulmonary fibrosis (IPF) is a devastating lung disease characterized by the accumulation of fibroblasts and extracellular matrix in the lungs. Despite extensive research and two currently FDA approved drugs, effective therapies for reversing fibrosis in IPF affected pulmonary tissues are not present. Therefore, there are unmet needs for treatment of IPF.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY OF DISCLOSURE

Disclosed herein, in certain aspects, include a modulator of a long noncoding RNA (lncRNA), wherein the lncRNA is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +), relative to human genome GRCh38.p14 (GCF_000001405.40).

In some instances, the RNA expression level of the lncRNA is at least about 2 folds, at least about 10 folds, or at least about 50 folds higher in the pulmonary myofibroblasts than in the pulmonary fibroblasts. In some instances, the region comprises one or more genetic variants selected from one or more single nucleotide polymorphisms (SNPs), indel variations, copy number variations (CNV), or a combination thereof. In some instances, the one or more SNPs comprises rs150797, rs220262, rs220249, rs2292305, rs3763267, rs2586502, rs61980882, rs37505950, or a combination thereof.

In some instances, the modulator is a nucleic acid editing or modifying moiety or a synthetic or artificial oligonucleotide. In some instances, the synthetic or artificial oligonucleotide comprises a nucleic acid sequence at least 80%, at least 90%, or at least 95% complementary to at least 10, at least 11, at least 12, at least 13, at least 14, or at least 15 contiguous nucleotides of the lncRNA. In some instances, the synthetic or artificial oligonucleotide is about 13-35, or about 16-20 nucleotides in length.

In some instances, the synthetic or artificial oligonucleotide comprises a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), or an enzymatically prepared siRNA (esiRNA) or a precursor thereof.

In some instances, the synthetic or artificial oligonucleotide is an ASO, wherein the ASO comprises i) a GapmeR comprising a central region of consecutive DNA nucleotides flanked by a 5′-wing region and 3′-wing region, wherein at least one of 5′-wing region and 3′-wing region comprises at least one nucleic acid analogue; and/or ii) a MixmeR of DNA and at least one nucleic acid analogue.

In some instances, the at least one nucleic acid analogue comprises an LNA or a 2′-methoxyethyl (MOE)-modified nucleotide. In some instances, the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA. In some instances, the 5′-wing region comprises at least two LNAs or 2′-methoxyethyl (MOE)-modified nucleotides. In some instances, the 5′-wing region comprises three, four, or five consecutive LNAs or 2′-methoxyethyl (MOE)-modified nucleotides. In some instances, the 3′-wing region comprises at least one LNA or a 2′-methoxyethyl (MOE)-modified nucleotide. In some instances, the 3′-wing region comprises two, three, four, or five consecutive LNAs or 2′-methoxyethyl (MOE)-modified nucleotides.

In some instances, at least one internucleotide linkage of the ASO is a phosphorothioate internucleotide linkage. In some instances, each internucleotide linkage of the ASO is a phosphorothioate internucleotide linkage.

In some instances, the modulator comprises a nucleic acid molecule comprising at least 10, at least 11, at least 12, or at least 13 consecutive nucleotides with no more than 1, no more than 2, or no more than 3 mismatches from any one of SEQ ID NOs: 1-2566 and 2580-3987, or comprises a nucleic acid sequence comprising at least 80%, at least 85%, at least 90%, or at least 95% identical to a sequence selected from any one of SEQ ID NOs: 1-2566 and 2580-3987. In some instances, the modulator comprises a nucleic acid molecule comprising at least 10, at least 11, at least 12, or at least 13 consecutive nucleotides with no more than 1, no more than 2, or no more than 3 mismatches from any one of SEQ ID NOs: 1-28, 31-37, 378, 385, 387, 2010-2018, and 2564-2566, or comprises a nucleic acid sequence at least 80%, at least 85%, at least 90%, or at least 95% identical to a sequence selected from any one of SEQ ID NOs: 1-28, 31-37, 378, 385, 387, 2010-2018, and 2564-2566. In some instances, the modulator comprises a nucleic acid molecule comprising at least 10, at least 11, at least 12, or at least 13 consecutive nucleotides with no more than 1, no more than 2, or no more than 3 mismatches from any one of SEQ ID NOs: 1-2013, 2564-2566, and 2580-2947, or comprises a nucleic acid sequence at least 80%, at least 85%, at least 90%, or at least 95% identical to a sequence selected from any one of SEQ ID NOs: 1-2013, 2564-2566, and 2580-2947. In some instances, the modulator comprises a nucleic acid molecule comprising at least 10, at least 11, at least 12, or at least 13 consecutive nucleotides with no more than 1, no more than 2, or no more than 3 mismatches from any one of SEQ ID NOs: 2014-2563 and 3948-3987, or comprises a nucleic acid sequence at least 80%, at least 85%, at least 90%, or at least 95% identical to a sequence selected from any one of SEQ ID NOs: 2014-2563 and 3948-3987.

In various aspects, the present disclosure provides a pharmaceutical composition comprising the modulator described herein and a pharmaceutically acceptable salt, excipient, or derivative thereof. In some instances, the modulator is encapsulated in liposome or coupled with a nanoparticle.

In some aspects, the present disclosure provides a method of modulating expression or activity of one or more pulmonary-specific lncRNAs in a subject, comprising administering an effective amount of the modulator described herein or the pharmaceutical composition described herein to the subject in need thereof, wherein the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +). In some instances, the modulator directly or indirectly modulates expression or activity of one or more pulmonary-specific lncRNAs in the subject.

In some instances, the modulator further modulates RNA expression level, protein expression level, or both, of one or more genes from Table 18. In some instances, the modulator reduces RNA expression level, protein expression level, or both, of the one or more genes from Table 18. In some instances, the modulator reduces RNA expression level, protein expression level, or both, of one or more fibrotic genes selected from a group consisting of COL3A1, COL1A1, ACTA2, POSTN, FN1, FAP, and CTHRC1.

In some instances, the modulator further modulates one or more genes selected from a group consisting of i) MX1, MX2, thrombospondin 1 (THBS1), thrombospondin 2 (THBS2), or combination thereof, ii) B9D1, IQCG, LRRC23, TMEM231, SPACA9, CCDC65, ARHGAP39, C9orf116, CFAP300, RAB36, or a combination thereof, iii) CCDC60, FAM81B, TCTE1, MDH1B, RSPH9, PACRG, C9orf116, ODAD4, TMEM231, or a combination thereof, and iv) CERCAM, COL3A1, COL1A1, COL5A2, COL1A2, CIS, ROR2, EPHB2, COL15A1, CTHRC1, or a combination thereof.

In some instances, the modulator restores a cytokine level to a level similar to a reference cytokine level obtained from a healthy subject. In some instances, the cytokine comprises IL-9, IL-1a, MIP-1a (CCL3), G-CSF, IL-2, IL-7, or a combination thereof.

In some instances, the modulator inhibits epithelial to mesenchymal transition (EMT) of pulmonary cells. In some instances, the pulmonary cells comprise pulmonary fibroblasts, pulmonary myofibroblasts, or epithelial cells.

In some instances, the administering is performed intratracheally, orally, nasally, intravenously, intraperitoneally, or intramuscularly. In some instances, the administering is in a form of aerosol.

In one aspect, the present disclosure provides a method of treating idiopathic pulmonary fibrosis (IPF) in a subject by administering the subject an effective amount of the modulator described herein or the pharmaceutical composition comprising the modulator described herein, thereby treating the idiopathic pulmonary fibrosis in the subject. In some instances, wherein the method further comprising: obtaining a sample of the subject; measuring from the sample (a) RNA expression level, protein expression level, or both, of a plurality of genes from a set of genes setting forth in Table 18; or (b) RNA expression level of one or more pulmonary-specific lncRNAs; and selecting the subject for the treatment based on (a) or (b).

In some instances, the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +).

In another aspect, the present disclosure provides a method of i) diagnosing idiopathic pulmonary fibrosis (IPF), or ii) monitoring, determining or predicting severity or progression of idiopathic pulmonary fibrosis (IPF) in a subject, the method comprising: (a) obtaining a sample from the subject; (b) determining an RNA expression level of (i) a plurality of genes selected from a group consisting of COL3A1, COL1A1, ACTA2, POSTN, FN1, FAP, CTHRC1, THBS1, THBS2, or (ii) one or more pulmonary-specific lncRNAs; and (c) diagnosing idiopathic pulmonary fibrosis (IPF), or monitoring, determining or predicting severity or progression of idiopathic pulmonary fibrosis (IPF) in the subject based on the measurement from step (b).

In some instances, determining the RNA expression level comprises comparing the RNA expression level of the set of genes from to a first reference RNA expression level. In some instances, the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +). In some instances, the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr17: 50199876-50215922 (strand +), and chr2: 215717811-215721161 (strand +).

In various aspects, the present disclosure provides a method of determining an efficacy of a treatment for idiopathic pulmonary fibrosis, comprising: comparing a first RNA expression level of a subject after the treatment and a second RNA expression level of a control, wherein the first and second RNA expression levels comprise an RNA expression level of (i) a plurality of genes selected from a group consisting of COL3A1, COL1A1, ACTA2, POSTN, FN1, FAP, CTHRC1, THBS1, THBS2, or (ii) one or more pulmonary-specific lncRNAs, In some instances, the control is a subject before the treatment, a healthy subject, or a reference sample not affected by idiopathic pulmonary fibrosis. In some instances, the subject is a cell, a tissue, an animal, or a human. In some instances, the pulmonary-specific lncRNAs are transcribed from a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +).

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings.

FIG. 1A shows evaluation of tissue-specificity by comparing in vitro pulmonary fibrosis induced non-coding RNA (PUFIN) expression to healthy lung genotype-tissue expression (GTEx expression) (in vivo) and untreated fibroblasts cells (in vitro). FIG. 1B shows evaluation of tissue-specific expression of these 12 PUFINs (named PUFIN1-PUFIN12) in lung tissue (GTEx). FIG. 1C shows that PUFINs, e.g., PUFIN2, PUFIN6, and PUFIN5, are endogenously expressed in healthy lung tissue (GTEx). FIG. 1D shows that PUFINs, e.g., PUFIN12, PUFIN1, and PUFIN11, are endogenously expressed in healthy lung tissue (GTEx). FIG. 1E shows that PUFIN1 expression is higher in myofibroblast (MyoFB) compared to fibroblast (FB) and is also higher in lung MyoFB than cardiac FB or cardiac MyoFB.

FIG. 2A shows a criteria summary to identify PUFIN1-12. FIG. 2B shows SNP associations with PUFIN1, PUFIN3-4, PUFIN6, PUFIN8, and PUFIN10. FIG. 2C show that PUFIN's (PUFIN1, PUFIN2, PUFIN6, PUFIN7, PUFIN8, PUFIN11, and PUFIN12) expression is higher in MyoFB cluster compared to FB cluster in a snRNAseq multi-omic dataset. FIG. 2D shows cell cluster results based on snRNA-Seq. FIG. 2E shows additional cell cluster results based on snRNA-seq as in FIG. 2D. FIG. 2F shows that the MyoFB cluster showed higher expression of canonical myofibroblast genes (COL3A1, COL1A1, ACTA2, POSTN, FN1, FAP) compared to FB cluster. FIG. 2G shows differentially expression of upregulated and downregulated genes in IPF in MyoFB cluster compared to the FB cluster.

FIG. 3A shows PUFIN1 and associated GWAS single-nucleotide polymorphism (SNPs). FIG. 3B shows an LD plot of SNPs in the PUFIN1 locus. FIG. 3C shows an LD plot of SNPs in the PUFIN3 locus.

FIG. 4A shows expression of PUFIN1 in IPF patients and controls. FIGS. 4B-4D show that PUFIN7 showed increased expression in IPF patients compared to healthy controls. FIG. 4B shows a diagram of a genomic locus transcribing PUFIN7. PUFIN7 is located near the locus transcribing FN1. FIG. 4C shows upregulation of FN1 and PUFIN7 expression in IPF patient. FIG. 4D shows a diagram of a of genomic locus transcribing PUFIN2. FIG. 4E shows that PUFIN2 and PUFIN3 are located upstream of THBS1. FIG. 4F shows a diagram of a of genomic locus transcribing PUFIN6. PUFIN6 is located upstream of COL1A1

FIG. 5A shows PUFIN1 expression in various tissues.

FIGS. 6A-6G show that expression of PUFIN1 along with canonical MyoFB markers upon treatment with LNA ASOs (qPCR and bulk RNAseq). FIG. 6A shows a diagram of genomic locus of PUFIN1 and targeting sequences of ASOs, ASO-1 (SEQ ID NO: 36) and ASO-2 (SEQ ID NO: 37). FIG. 6B shows graphs illustrating expression level of PUFIN1 and canonical fibrotic markers upon ASO-1 or ASO-2 treatment. FIG. 6C shows graphs illustrating expressions of PUFIN1 and canonical fibrotic markers upon siRNA treatment. FIG. 6D shows dose dependent expression levels of PUFIN1 and canonical fibrotic marker expression levels upon ASO treatment. FIG. 6E shows a diagram of a genomic locus transcribing PUFIN1, overview of epigenetic regulation, and overview of expression upon ASO1 treatment. FIG. 6F shows expression levels of PUFIN1 and IPF gene signature from FB and upon scramble ASO treatment or ASO1 treatment. FIG. 6G shows expression levels of genes from target engagement panel upon ASO1 or scramble ASO treatment. FIG. 6H shows that PUFIN1 shares a topologically associating domain (TAD) with the genes located downstream of PUFIN1.

FIG. 7A shows a schematic diagram of experimental procedures using mouse models (bleomycin model and transchromosomic chromosome 21 (TcMAC21) bleomycin model) to test ASO1 targeting PUFIN1. FIG. 7B show graphs of body weight changes and lung weight changes in the mouse models of FIG. 7A with or without ASO treatment. FIG. 7C shows graphs of leukocyte and immune cell distribution in the mouse models of FIG. 7A with or without ASO treatment. FIG. 7D shows immunohistochemical images and graphs indicating fibrotic foci development upon bleomycin (Bleo) treatment, which is attenuated upon ASO-1 treatment. FIG. 7E shows graphs of hsPUFIN1 expression in mouse model. FIG. 7F shows graphs of pro-fibrotic gene expression (left panel) and IPF gene expression (right panel) in mouse model. FIG. 7G shows results of GSEA pathway analysis, ciliated pathways, and genes associated with hsPUFIN1 downregulation in after ASO-1 treatment in the bleomycin model.

FIGS. 8A-8G show expression of PUFIN7 lncRNA and fibrotic genes and modulating of expression by treatment with ASOs. FIG. 8A shows a schematic diagram of PUFIN7 location and expression, and target locations of ASOs targeting PUFIN7 (LNA1-LNA9). FIG. 8B shows a graph of expression levels of PUFIN7 in primary human lung fibroblasts (pHLF) upon ASO treatment. FIG. 8C shows graphs of expression of fibrotic gene markers upon PUFIN7 ASO treatment. FIG. 8D shows a brightfield image of pHLF. FIG. 8E shows expression of PUFIN7 and IPF gene signature upon ASO treatment. FIG. 8F shows expression of fibrotic markers, e.g., ACTA2, COL1A1, and COL3A1 upon ASO treatment. FIG. 8G shows expression of fibrotic markers, e.g., FAP, FN1, and POSTN upon ASO treatment. FIGS. 8H and 8I show co-expressed genes associated with PUFIN7 knock-down.

FIGS. 9A-9C show decrease in PUFIN3 expression and fibrotic marker genes upon ASO treatment. FIG. 9A top panel shows schematic diagram of PUFIN3 location and expression, and target locations of ASOs targeting PUFIN3 (LNA1-LNA7). FIG. 9A bottom panel shows PUFIN3 expression upon ASO (LNA1-7 or L1-7) treatment. FIG. 9B shows a graph of THBS1 and THBS2 expression upon ASO treatment targeting PUFIN3. FIG. 9C shows graphs of fibrotic markers expression upon ASO treatment targeting PUFIN3.

FIGS. 10A-10C show biomarker concentrations collected from ex vivo human precision cut lung slides (hPCLS) model using immunoassay. FIG. 10A shows graphs of concentration of biomarkers: MMP-1, MMP-7, MMP-3, TIMP-1, pro-collagen I alpha 1, and fibronectin. FIG. 10B shows graphs of concentration of biomarkers: VEGF, IL-2, CCL2/MCP-1, IL-7, IL-6, and IL-8. FIG. 10C shows graphs of concentration of biomarkers: PAI-1, collagen IV alpha 1, and thrombospondin 2.

FIGS. 11A-11G show RNA sequencing data of samples from the ex vivo hPCLS model. FIG. 11A shows a graph of IPF gene score from different timepoint and conditions. FIG. 11B shows graphs of expression of PUFIN1, PUFIN2, and PUFIN3. FIG. 11C shows graphs of expression of PUFIN4, PUFIN5, PUFIN6, and PUFIN7. FIG. 11D shows graphs of expression of PUFIN8, PUFIN9, and PUFIN12. FIG. 11E shows graphs of expression of fibrotic markers: ACTA2, COL1A1, and COL3A1. FIG. 11F shows graphs of expression of fibrotic markers: FAP, FN1, and POSTN. FIG. 11G shows expression of THBS1 and THBS2.

FIGS. 12A-12E show graphs of gene expression of PUFIN6 (FIG. 12A), COL1A1 (FIG. 12B), COL3A1 and FN1 (FIG. 12C), CTHRC1 and FAP (FIG. 12D), POSTN and ACTA2 (FIG. 12E) upon ASO treatment. FIG. 12F shows ProSeq data of PUFIN6 lncRNA.

FIGS. 13A-13F show graphs of gene expression of PUFIN2 (FIG. 13A), THBS1 and THBS2 (FIG. 13B), COL3A1 and COL1A1 (FIG. 13C), POSTN and CTHRC1 (FIG. 13D), FN1 and ACTA2 (FIG. 13E), and FAP (FIG. 13F) upon ASO treatment.

FIGS. 14A-14C show graphs of gene expression of PUFIN4 (FIG. 14A), ACTA2, FN1, FAP, and POSTN (FIG. 14B), COL1A1, COL3A1, and CTHRC1 (FIG. 14C) upon ASO treatment.

FIGS. 15A-15C show graphs of gene expression of PUFIN8 (FIG. 15A), COL3A1, COL1A1, FAP, CTHRC1, ACTA2, FN1 (FIG. 15B), and POSTN (FIG. 15C) upon ASO treatment.

FIG. 16A shows identification of PUFIN1 as a lncRNA transcript transcribed from a lung super-enhancer, with a RUNX1 motif located within PUFIN1 promoter. FIG. 16B shows an overview of epigenetic profile of PUFIN1 and correlation of PUFIN1 expression in human lung fibroblasts (HLFs) with super-enhancer marked by H3K27Ac. FIG. 16C shows results of bulk RNA-seq of IPF gene signature and PUFIN1 TEP evaluation in mice model. FIGS. 16D-16E show images of picrosirius red staining (FIG. 16D) and H&E staining (FIG. 16E) of samples from TcMAC21 mice model. FIG. 16F shows a graph of PUFIN1 expression upon TGF-β treatment using in vitro model. FIG. 16G shows graphs of PUFIN1 expression in IPF patients from single-cell RNA-seq data. FIGS. 16H-16I show that in vitro fibroblast differentiation using TGF-β resulted in activated myofibroblasts that expressed higher levels of canonical profibrotic marker genes, which are displayed in heatmap (FIG. 16H). Some of canonical profibrotic marker genes, e.g., FOSL1, COL1A1, ACTA2, TGFB1, MMP2, HAS2 were shown in FIG. 16I.

FIG. 17A shows a diagram of genomic location transcribing PUFIN7, which is near fibronectin (FN1). FIG. 17B shows a diagram of PUFIN7 enrichment in lung myofibroblasts and expression of PUFIN7 upon LNA6 (PUF7_LNA6) and PUFIN7 LNA9 (PUF7_LNA9) treatment.

FIG. 18A shows a diagram of PUFIN3 expression in myofibroblast, and upon PUFIN3 ASO treatment, PUFIN3 LNA3 (PUF3_LNA3) or PUFIN3 LNA1 (PUF3_LNA1). FIG. 18B shows PUFIN3 quantification from bulk RNA-seq upon ASO treatment.

FIG. 19A shows graphs of relative dose-dependent PUFIN1 expression upon ASO treatment (left panel). IPF gene signature (shown in Table 18) (right panel) was downregulated in a dose-dependent manner by ASO1 treatment. FIG. 19B shows a graph of dose-responsive PUFIN1 96 TEP gene expression upon ASO1 treatment. FIGS. 19C-19D show graphs of PUFIN1 TEP gene expression in IPF patient bulk RNAseq data. FIG. 19E shows graphs of dose dependent expression of CADM1, COL3A1, NTM, and PFN2 in the PUFIN1 TEP. FIG. 19F shows graphs of dose dependent expression of ELN, FBXL7, and SFRP4 in the PUFIN1 TEP. FIG. 19G shows PUFIN1 expression, PUFIN1 96 TEP genes, and IPF gene signature (genes listed in Table 18) in a human ex vivo advanced fibrosis model (as analyzed using IPF patient tissue data). FIG. 19H shows a boxplot of the PUFIN1 TEP that was increased in a fibrotic cocktail condition (FC) compared to either control (CC) or anti-fibrotic treatment (FC+Nintedanib).

FIG. 20A shows a dose response curve of PUFIN7 expression upon LNA6 (or L6) ASO treatment (left panel). IPF gene signature (genes listed in Table 18) (right panel) was downregulated in a dose-dependent manner by LNA6 ASO treatment. FIG. 20B shows a graph of PUFIN7 TEP gene expression in a dose-responsive manner upon LNA6 treatment. FIG. 20C shows graphs of IPF related gene expression e.g., collagen-containing extracellular matrix (COL3A1, COL5A1, COL6A3, ELN, TNC, MXRA5, FBN1), responded in a dose dependent manner upon PUFIN7 L6 ASO treatment. FIG. 20D shows graphs of IPF related gene expression, e.g., actin polymerization or depolymerization (DIAPH2, AVIL, GAS7), and pulmonary valve development (SMAD2, ROBO1), responded in a dose dependent manner upon PUFIN7 L6 ASO treatment. FIGS. 20E-20F show graphs of PUFIN7 TEP gene expression in IPF patient by bulk RNAseq data (FIGS. 20E-20F). FIG. 20G shows graphs of PUFIN7 expression, PUFIN7 96 TEP genes, and IPF Gene Signature (genes listed in Table 18) in a human ex vivo advanced fibrosis model (as analyzed using IPF patient tissue data). PNA33 is a peptide nucleic acid inhibitor for mir-33. FIG. 20H shows a boxplot of the PUFIN7 TEP which was increased in a fibrotic cocktail condition (FC) compared to either control (CC) or anti-fibrotic treatment (FC+Nintedanib). PUFIN7 TEP was significantly downregulated after the treatment of Nintedanib.

FIG. 21A shows graphs of PUFIN3 and IPF signature expression upon PUFIN3 LNA1 (or L1) ASO treatment (left panel). FIG. 21B shows a graph of PUFIN3 TEP gene expression. FIG. 21C shows graphs of PUFIN3 TEP gene expression, which are related to cell-cell adhesion pathways and the expression of Wnt receptor activity pathways. FIG. 21D shows graphs of genes PUFIN3 TEP gene expression, which are related to ciliary neurotrophic factor receptor binding and metalloendopeptidase activity. FIGS. 21E-21F show graphs of PUFIN3 TEP gene in IPF patient based on analysis of bulk RNAseq data (FIGS. 21E-21F). FIG. 21G shows graphs of PUFIN3 expression, PUFIN3 TEP genes, and IPF Gene Signature (genes listed in Table 18) in a human ex vivo advanced fibrosis model (as analyzed using IPF patient tissue data). FIG. 21H shows a boxplot of the PUFIN3 TEP among different conditions.

FIG. 22A shows a Venn diagram displaying the overlapping genes between genes in PUFIN1 TEP, genes in PUFIN3 TEP, and genes in PUFIN7 TEP.

FIGS. 23A-23B show graphs of PUFIN7 response in a dose dependent manner upon PUFIN1 ASO1 treatment (FIG. 23A) and upon PUFIN3 L1 ASO treatment (FIG. 23B). FIG. 23C shows graphs of LINC01239 expression upon PUFIN1 ASO1 treatment (left panel), PUFIN3 L1 ASO treatment (middle panel), and PUFIN 7 L6 ASO treatment (right panel). FIG. 23D shows graphs of gene expressions associated with PUFIN1 TEP, PUFIN3 TEP, and PUFIN7 TEP in fibrotic ex vivo hPCLS model.

### DETAILED DESCRIPTION

The present disclosure provides that long noncoding RNA (lncRNA) that are associated with onset, development, or prognosis of idiopathic pulmonary fibrosis (IPF) are identified, and that differential expression or transcriptional regulation of the lncRNA are associated with symptoms, development, or prognosis of IPF. In some aspects, the lncRNAs are associated with onset or development of pulmonary fibrosis, e.g., IPF, or a symptom of IPF. Such lncRNAs described herein can be utilized as biomarkers to predict severity and progression of IPF in a subject. Further, such lncRNAs described herein could be a druggable target to prevent, alleviate, or treat IPF. In some instances, modulation of such lncRNA described herein can improve a subject's conditions associated with IPF. As such, in one aspect, provided herein, are modulators of a lncRNA associated with onset, development or prognosis of IPF, or associated with onset or development of pulmonary fibrosis, e.g., IPF, or a symptom of IPF. Also, the present disclosure provides modulators or pharmaceutical compositions comprising the modulator, and kits comprising the modulator. Also provided herein are methods of modulating a lncRNA associated with onset, development, or prognosis of IPF or associated with onset or development of IPF or a symptom of IPF in a subject in need thereof. Further provided herein are methods of preventing, alleviating, or treating IPF or preventing, alleviating, or treating pulmonary fibrosis or inflammation in a subject affected by IPF. In another aspect, provided herein are methods of predicting severity and progression of idiopathic pulmonary fibrosis (IPF) in a subject.

Long Noncoding RNAs (lncRNAs) Associated with Idiopathic Pulmonary Fibrosis

Idiopathic pulmonary fibrosis (IPF) is a chronic life-threatening lung disease that is characterized by the scarring or fibrosis of lung, resulting in lung stiffness which affect breathing and oxygen delivery from alveoli to bloodstream. The histological hallmarks of IPF comprise excessive deposition of extracellular matrix protein (ECM) with presence of fibroblast loci and spatially heterogeneous fibrotic area. IPF is considered an age-related disorder with the unknown cause. Damaged lung tissues in IPF are irreversible and get worse over time. The disease pathogenesis of IPF involves injury to alveolar epithelium, chronic activation of inflammatory cells and fibroblasts, and abnormal tissue repairing/remodeling, which results in lung fibrosis.

Long non-coding RNAs (or long non-coding transcripts or lncRNAs) are RNA segments that lack protein-coding capacity, yet mediate various regulatory mechanisms in cell cycle or cell metabolism by regulating transcription and/or post-transcriptional modification of various genes. As such dysregulation of certain lncRNAs can be associated with an onset, development, or prognosis of a disease or a symptom of a disease. Additionally, dysregulation of certain lncRNAs can be a signature or indication of an onset, development, or prognosis of a disease or a symptom of a disease.

Some lncRNAs affect development of certain types of fibrosis by promoting extracellular matrix (ECM) synthesis by affecting cells, such as fibroblasts or myofibroblasts, in the tissue(s). Long non-coding RNAs (lncRNAs) are critical regulators of gene expression and play roles as master-regulators of cell state transition and transcription in IPF. In some aspects, provided herein are certain lncRNA is associated with onset, development, and/or prognosis of IPF. In some aspects, certain lncRNA is associated with onset, development, and/or prognosis of pulmonary fibrosis, e.g., IPF. In some aspects, the pulmonary fibrosis is induced by IPF. In some aspects, the pulmonary fibrosis has resulted from one or more symptoms or pathophysiology of IPF.

In some aspects, the expression of the lncRNAs disclosed herein is elevated in a subject affected by pulmonary fibrosis, e.g., IPF. In some aspects, the expression of the lncRNAs disclosed herein is elevated in a subject affected by IPF. In some aspects, the expression of the lncRNAs disclosed herein is elevated in a subject having an inflammation or increased immune response associated with onset or development of IPF. In some aspects, the inflammation or increased immune response is represented or shown by increased infiltration of immune cells to the tissue (e.g., lung or pulmonary tissue), activation of immune cells in the lung tissue or to the lung tissue (e.g., lung or pulmonary fibroblast), increased secretion or accumulation of inflammatory cytokines or chemokines in the lung tissue. In some aspects, the elevation is at least by 1%, 2%, 3%, 4%, 5%, 10%, 20%, 30%, 40%, 50%, 60%, 70%, 80%, 90%, 100%, 200%, 300%, 400%, 500%, 600%, 700%, 800%, 900%, or 1000%. In some aspects, the activity of the lncRNAs disclosed herein is elevated in a subject affected by fibrosis associated with IPF. In some aspects, the elevation is at least by 1%, 2%, 3%, 4%, 5%, 10%, 20%, 30%, 40%, 50%, 60%, 70%, 80%, 90%, 100%, 200%, 300%, 400%, 500%, 600%, 700%, 800%, 900%, or 1000%.

In some aspects, the elevated expression and/or activity of the lncRNA disclosed herein is associated with severity of an IPF symptom in the subject. In some aspects, the IPF symptom comprises chest pain or tightness, shortness of breath (dyspnea), dry cough, tiredness, aching muscles and joints, loss of appetite, weight loss, or widening and rounding of the tips of fingers or toes (clubbing). In some aspects, the elevated expression is associated with severity of chest pain or tightness. In some aspects, the elevated expression is associated with severity of shortness of breath. In some aspects, the elevated expression is associated with severity of dry cough. In some aspects, the elevated expression is associated with severity of tiredness.

In some aspects, the elevated expression and/or activity of the lncRNA disclosed herein is detected in pulmonary myofibroblasts compared to pulmonary fibroblasts obtained from a same individual. In some aspects, the elevated expression and/or activity of the lncRNA disclosed herein is detected in the induced pulmonary myofibroblasts compared to pulmonary fibroblasts obtained from a same individual. In some aspects, the expression level of the lncRNA is increased in the pulmonary myofibroblasts or induced pulmonary myofibroblasts at least 10%, 20%, 30%, 40%, 50%, 60%, 70%, 80%, 90%, 100%, 200%, 300%, 400%, 500% compared to pulmonary fibroblasts.

In some aspects, the elevated expression and/or activity of the lncRNA disclosed herein is detected in the pulmonary myofibroblasts obtained from a subject with IPF compared to pulmonary fibroblasts obtained from a healthy subject. In some aspects, the elevated expression and/or activity of the lncRNA disclosed herein is detected in the induced pulmonary myofibroblasts obtained from a subject with IPF compared to pulmonary fibroblasts obtained from a healthy subject. In some aspects, the expression level of the lncRNA is increased in the pulmonary myofibroblasts or induced pulmonary myofibroblasts obtained from a subject with IPF at least 10%, 20%, 30%, 40%, 50%, 60%, 70%, 80%, 90%, 100%, 200%, 300%, 400%, 500% compared to pulmonary fibroblasts obtained from a healthy subject. In some aspects, the normal pulmonary fibroblasts are healthy fibroblasts or non-diseased fibroblasts.

In some aspects, the lncRNA disclosed herein is associated with or modulates expression or activity of one or more fibrotic marker genes. In some instances, the one or more fibrotic marker genes comprise smooth muscle a actin (ACTA2), alpha 1 chain of collagen type I (COL1A1), alpha 1 chain of collagen type 3 (COL3A1), fibroblast activation protein (FAP), fibronectin 1 (FN1), periostin (POSTN), Collagen Triple Helix Repeat Containing 1 (CTHRC1), thrombospondin 1 (THBS1), thrombospondin 2 (THBS2), or a combination thereof. In some instances, the increased expression of lncRNA disclosed herein is proportional to the increased expression of the one or more fibrotic marker genes.

In some aspects, the lncRNA disclosed herein is transcribed from a genomic region located at the same chromosome with one or more fibrotic marker genes. In some aspects, the lncRNA is transcribed from the genomic region located at chromosome 2 and the one or more fibrotic markers genes comprise fibronectin 1 (FN1).

In some aspects, the lncRNA disclosed herein is transcribed from a genomic region where one or more single nucleotide polymorphisms (SNPs) that are related to pathology of IPF are located. In some aspects, the lncRNA disclosed herein is transcribed from a genomic allele where one or more single nucleotide polymorphisms (SNPs) associated with IPF are located. As used herein, SNPs associated with IPF is any genetic or epigenetic variation, mutation, or modification that contributes to IPF susceptibility, severity, and/or mortality. In some aspects, the one or more SNPs related to pathology of IPF or associated with IPF comprise one or more SNPs that are associated with susceptibility to pulmonary fibrosis (e.g., IPF), immune function (e.g., lymphocyte count), lung or pulmonary function (e.g., force vital capacity), systolic blood pressure, response to taxane treatment (e.g., docetaxel), or increase of expression of fibrotic marker (e.g., THBS2), or combination thereof. In some aspects, a certain aspect of IPF susceptibility, severity, and/or mortality relates to shortness of breath. In some aspects, a certain aspect of IPF susceptibility, severity, and/or mortality relates to difficulty in breathing. In some instances, the one or more SNPs that are related to pathology of IPF or associated with IPF are identified from genome-wide association study (GWAS).

In some aspects, the one or more SNPs are associated with pulmonary fibrosis (e.g., IPF) risk. In some aspects, the one or more SNPs are associated with IPF risk variants. In some aspects, the IPF risk variants comprise SNPs listed in any one of Tables 46-57. In some aspects, the one or more SNPs are associated with IPF transplant free survival variants. In some aspects, the IPF transplant free survival variants comprises SNPs listed in any one of Tables 58-69.

In some aspects, the genomic region in which the lncRNA are transcribed as disclosed herein is annotated according to the Human Genome Resources at NCBI. In some aspects, the genomic regions in which the lncRNAs are transcribed as disclosed herein are annotated according to the Human Genome Resources at NCBI Genome Assembly: GRCh38.p14 (GCF_000001405.40). In some aspects, the genomic regions in which the lncRNAs are transcribed as disclosed herein are located within chromosome 21, chromosome 15, chromosome 6, chromosome 4, chromosome 17, chromosome 2, chromosome 14, chromosome 11, or chromosome 16.

In some aspects, the lncRNA is transcribed from a region located within a genomic locus selected from a group consisting of chr21: 42,009,177-42,055,325; chr15: 38,864,806-39,427,195; chr15: 38,871,616-38,880,364; chr6: 169,171,312-169,188,635; chr4: 76,758,554-76,801,964; chr17: 50,199,876-50,215,922; chr2: 215,717,811-215,721, 161; chr14: 74,552,181-74,560,688; chr2: 188,966,458-188,984,794; chr11: 1,218,530-1,220,242; chr6: 74,069,451-74,690,727; and chr16: 86,845,338-87,058,332. In some aspects, the lncRNA is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +), relative to human genome GRCh38.p14 (GCF_000001405.40). In some aspects, the lncRNAs is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); and chr16: 86845338-87058332 (strand −), relative to human genome GRCh38.p14 (GCF_000001405.40). In some aspects, the lncRNAs is transcribed from a region located within a genomic locus selected from a group consisting of ENSG00000237232.9 (chr21: 42009177-42055325), ENSG00000259345.7 (chr15: 38864806-39427195), XLOC_029593 (chr15: 38871616-38880364), ENSG00000261039.3 (chr6: 169171312-169188635), ENSG00000224218.1 (chr4: 76758554-76801964), ENSG00000249406.3 (chr17: 50199876-50215922), ENSG00000230838.2 (chr2: 215717811-215721161), ENSG00000258425.1 (chr14: 74552181-74560688), XLOC_051798 (chr2: 188966458-188984794), ENSG00000286275.1 (chr11: 1218530-1220242), ENSG00000223786.1 (chr6: 74069451-74690727), and ENSG00000289423.1 (chr16: 86845338-87058332).

In some aspects, the lncRNA disclosed herein comprises a nucleic acid sequence of at least a portion of a lncRNA transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +).

In some aspects, the lncRNA disclosed herein comprises a nucleic acid sequence of at least a portion of lncRNA transcribed from a region located within a genomic locus selected from a group consisting of ENSG00000237232.9, ENSG00000259345.7, XLOC_029593, ENSG00000261039.3, ENSG00000224218.1, ENSG00000249406.3, ENSG00000230838.2, ENSG00000258425.1, XLOC_051798, ENSG00000286275.1, ENSG00000223786.1, and ENSG00000289423.1 (Ensembl/Gencode). In some aspects, the lncRNA disclosed herein comprises a nucleic acid sequence of RNA transcripts transcribed from a region located within a genomic locus selected from a group consisting of ENSG00000237232.9, ENSG00000259345.7, XLOC_029593, ENSG00000261039.3, ENSG00000224218.1, ENSG00000249406.3, ENSG00000230838.2, ENSG00000258425.1, XLOC_051798, ENSG00000286275.1, ENSG00000223786.1, and ENSG00000289423.1 (gene ID based on Ensembl/Gencode). In some aspects, the lncRNA disclosed herein comprises a nucleic acid sequence of RNA transcripts transcribed from a region located within a genomic locus selected from a group consisting of ENSG00000237232.9, ENSG00000259345.7, XLOC_029593, ENSG00000261039.3, ENSG00000224218.1, ENSG00000249406.3, ENSG00000230838.2, ENSG00000258425.1, XLOC_051798, ENSG00000286275.1, ENSG00000223786.1, and ENSG00000289423.1 (Ensembl/Gencode).

In some aspects, the lncRNA disclosed herein comprises a nucleic acid sequence of at least a portion of RNA transcripts transcribed from a region located within a genomic locus selected from a group consisting of ZNF295-AS1, ENSG00000259345, ENSG00000261039.3, ENSG00000224218.1, ENSG00000249406, LINC01614, ENSG00000258425.1, ENSG00000286275, ENSG00000223786, and ENSG00000289423. In some aspects, the lncRNA disclosed herein comprises a nucleic acid sequence of RNA transcripts transcribed from a region located within a genomic locus selected from a group consisting of ZNF295-AS1, ENSG00000259345, ENSG00000261039.3, ENSG00000224218.1, ENSG00000249406, LINC01614, ENSG00000258425.1, ENSG00000286275, ENSG00000223786, and ENSG00000289423.

In some aspects, the lncRNA described herein is a pulmonary-specific lncRNA. As described herein, in some instances, the pulmonary-specific lncRNA refers to lncRNAs that are expressed in the lung of a subject with IPF. In some instances, the pulmonary-specific lncRNA refers to lncRNAs that are overexpressed in the lung of a subject with IPF when compared to the lung of healthy subject. In some aspects, the pulmonary-specific lncRNA refers to pulmonary fibrosis induced non-coding RNA (PUFIN) as identified in Table 17. In some aspects, the pulmonary-specific lncRNA is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +). In some aspects, the pulmonary-specific lncRNA is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); and chr16: 86845338-87058332 (strand −). In some aspects, the pulmonary-specific lncRNA is associated with pathology of IPF. In some aspects, the pulmonary-specific lncRNA is associated with IPF.

In some aspects, RNA expression level of the pulmonary-specific lncRNA is higher in pulmonary myofibroblasts compared to RNA expression level of the pulmonary-specific lncRNA in pulmonary fibroblasts. In some instances, the RNA expression level of the lncRNA is at least about 2 folds, at least about 5 folds, at least about 10 folds, at least about 20 folds, at least about 30 folds, at least about 40 folds, or at least about 50 folds higher in the pulmonary myofibroblasts than in the pulmonary fibroblasts. In some instances, the RNA expression level of the lncRNA is at least about 2 folds, at least about 10 folds, or at least about 50 folds higher in the pulmonary myofibroblasts than in the pulmonary fibroblasts. In some aspects, RNA expression level of the pulmonary-specific lncRNA is at least about 10%, at least about 20%, at least about 30%, at least about 40%, at least about 50%, at least about 60%, at least about 70%, at least about 80%, at least about 90%, at least about 2 folds, at least about 5 folds, at least about 10 folds, at least about 20 folds, at least about 30 folds, at least about 40 folds, or at least about 50 folds higher in the pulmonary myofibroblasts than in the pulmonary fibroblasts. In some aspects, RNA expression level of the pulmonary-specific lncRNA is at least 10 folds, at least 20 folds, or at least 50 folds higher in the pulmonary myofibroblasts than in the pulmonary fibroblasts. In some aspects, RNA expression level of the pulmonary-specific lncRNA is higher in pulmonary myofibroblasts obtained from a subject with IPF compared to RNA expression level of the pulmonary-specific lncRNA in pulmonary fibroblasts obtained from a healthy subject. In some aspects, RNA expression level of the pulmonary-specific lncRNA is at least about 10%, at least about 20%, at least about 30%, at least about 40%, at least about 50%, at least about 60%, at least about 70%, at least about 80%, at least about 90%, at least about 2 folds, at least about 5 folds, at least about 10 folds, at least about 20 folds, at least about 30 folds, at least about 40 folds, or at least about 50 folds higher in the pulmonary myofibroblasts obtained from the subject with IPF than in the pulmonary fibroblasts obtained from the healthy subject. In some aspects, RNA expression level of the pulmonary-specific lncRNA is at least 10 folds, at least 20 folds, or at least 50 folds higher in the pulmonary myofibroblasts obtained from the subject with IPF than in the pulmonary fibroblasts obtained from the healthy subject.

In some aspects, the genomic locations encoding the pulmonary-specific lncRNAs comprise one or more genetic variants. In some instances, the region comprises one or more genetic variants selected from one or more single nucleotide polymorphisms (SNPs), indel variations, copy number variations (CNV), or combination thereof. In some aspects, the one or more genetic variants comprise one or more single nucleotide polymorphisms (SNPs), indel variations, copy number variations (CNV), or combination thereof. In some aspects, the one or more SNPs are associated with susceptibility to pulmonary fibrosis (e.g., IPF), immune function (lymphocyte count), lung function (e.g., forced vital capacity), systolic blood pressure, response to taxane treatment (e.g., docetaxel), or a combination thereof. In some aspects, the one or more SNPs are associated with pathology of IPF. In some aspects, the one or more SNPs are associated with IPF. In some aspects, the one or more SNPs are identified in GWAS studies. In some aspects, the one or more SNPs comprise rs150797, rs220262, rs220249, rs2292305, rs3763267, rs2586502, rs61980882, rs37505950, or combination thereof. In some instances, the one or more SNPs comprises SNPs listed in any one of Tables 40-69.

### Modulator of Long Noncoding RNA

In one aspect, the present disclosure provides a modulator of long noncoding RNAs (lncRNAs) described herein. In some aspects, the modulator disclosed herein modifies the genomic DNA that is transcribed to the lncRNA disclosed herein. In some aspects, the modulator disclosed herein modifies a portion of such genomic DNA so that the genomic DNA is mutated. In some aspects, the modulator disclosed herein modifies a portion of such genomic DNA so that the lncRNA transcription is suppressed. In some aspects, the modulator reduces the amount of the lncRNAs or reduces the RNA expression of the lncRNAs. In some aspects, the modulator disclosed herein modifies a portion of such genomic DNA so that the transcription level is activated. In some aspects, the modulator disclosed herein modifies the lncRNAs disclosed herein. In some aspects, the modulator disclosed herein modifies a portion of the lncRNAs so that the lncRNAs are degraded. In some aspects, the modulator disclosed herein modifies a portion of the lncRNAs so that the lncRNAs are retained longer. In some aspects, the modulator disclosed herein modifies a portion of the lncRNAs so that the activity of the lncRNAs on downstream reactions or pathways is suppressed. In some aspects, the modulator reduces the activity of the lncRNAs. In some instances, the modulator disclosed herein modifies a portion of the lncRNAs so that the activity of the lncRNAs on downstream reactions or pathways is activated.

In some aspects, a modulator of the lncRNAs increases or decreases of the expression or activity of the lncRNAs. In some aspects, a modulator of the lncRNAs prevent increases or decreases of the expression or activity of the lncRNAs. In some aspects, a modulator of the lncRNAs prevents the pathological changes associated with the expression or activity of the lncRNA. In some aspects, a modulator of the lncRNAs slows down the pathological changes associated with the expression or activity of the lncRNAs at least ±5%, at least ±10%, at least ±15%, at least ±20%, at least ±25% of the normal expression or activity of the lncRNAs before the onset or development of the pathological symptoms or diseases (e.g., baseline level), or at least ±5%, at least ±10%, at least ±15%, at least ±20%, at least ±25% of the expression or activity of the lncRNAs of a healthy subject or healthy tissue (e.g., baseline level). In some aspects, the modulator disclosed herein reduces the elevated amount of lncRNAs by at least 30%, at least 40%, at least 50%, at least 60%, at least 70%, at least 80%, or at least 90% in a cell or a tissue of the subject, wherein the elevated amount is associated with IPF. In some aspects, the modulator disclosed herein reduces the elevated activity of the lncRNAs by at least 30%, at least 40%, at least 50%, at least 60%, at least 70%, at least 80%, or at least 90% in a cell or a tissue of the subject, wherein the elevated amount is associated with IPF. In some aspects, the modulator described herein reduces the RNA expression level of the lncRNAs at least 30%, at least 40%, at least 50%, at least 60%, at least 70%, at least 80%, or at least 90% in a cell or a tissue of the subject with IPF.

In some aspects, the lncRNA modulates gene expression of one or more downstream gene. In some aspects, the gene expression of one or more downstream genes comprises expression of a marker for a disease onset, development, or prognosis. In some aspects, the modulator of the lncRNA modulates (e.g., increases or decreases) the expression or activity of the lncRNA or the one or more downstream genes. In some aspects, the modulator of the lncRNA increases the expression or activity of the one or more downstream genes at least 10%, at least 20%, at least 30%, at least 40%, at least 50%, at least 60%, at least 70%, at least 80%, at least 90%, or higher. In some aspects, the modulator of the lncRNA decreases the expression or activity of the one or more downstream genes at least 10%, at least 20%, at least 30%, at least 40%, at least 50%, at least 60%, at least 70%, at least 80%, at least 90%, or more. In some aspects, the modulator of the lncRNA prevents the pathological changes associated with the expression or activity of the one or more downstream genes. In some aspects, the modulator of the lncRNA slows down the pathological changes associated with the expression or activity of the one or more downstream genes. In some aspects, the downstream gene is a marker gene for pulmonary fibrosis, e.g., IPF. In some aspects, the downstream gene is a marker gene for IPF.

In some aspects, the modulator modulates RNA expression level of lncRNA. In some aspects, the modulator prevent transcription of the lncRNA. In some aspects, the modulator reduces RNA expression level of the lncRNA. In some aspects, the lncRNA is a pulmonary-specific lncRNA. In some aspects, the modulator reduces RNA expression level of the pulmonary-specific lncRNA. In some aspects, the pulmonary-specific lncRNA is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +). In some aspects, the pulmonary-specific lncRNA is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); and chr16: 86845338-87058332 (strand −). In some aspects, the pulmonary-specific lncRNA is associated with pathology of IPF. In some aspects, the pulmonary-specific lncRNA is associated with IPF.

In some aspects, the modulator reduces RNA expression level of the lncRNAs. In some aspects, the lncRNAs are pulmonary-specific lncRNAs. In some aspects, the pulmonary-specific lncRNAs are associated with IPF.

In some aspects, the lncRNAs described herein comprise one or more isoforms. In some aspects, the one or more isoforms share one or more exons that are similar in sequences (or conserved exon). As described herein, in some aspects, the modulator targets a region of the lncRNA. In some aspects, the region of the lncRNA comprises a conserved exon shared among a plurality of isoforms of the lncRNA. In some aspects, the modulator targets a region of the lncRNA that is unique to a specific isoform.

In some aspects, the marker gene for IPF is associated with autophagy, ubiquitin, cilium and flagellum assembly and function, extracellular matrix (e.g., collagen) synthesis, metabolism, deposition, and organization, cell organization, genome regulation, or ncRNA and mRNA regulation. In some aspects, the marker gene for IPF comprises one or more fibrotic genes. In some aspects, the marker gene for IPF comprises IPF gene signatures (Singscore—a group of genes), a subset thereof (e.g., at least 3, at least 5, at least 7, at least 10, at least 20, at least 30, at least 40, or at least 50 genes selected from the group of genes), or a specific subset thereof (e.g., at least 3, at least 5, at least 7, at least 10, at least 20, at least 30, at least 40, or at least 50 genes associated with specific phenotypes of cells, specific cellular pathway, specific signaling pathway, etc.), which are listed in Table 18. In some aspects, the IPF gene signatures are human IPF gene signatures constructed from expression data derived from human cells. In some aspects, the IPF gene signatures are constructed according to Example 1 described herein. In some aspects, the IPF signature listed in Table 18 are genes that distinguish IPF from other conditions, e.g., healthy control or other lung pathologies such as acute lung injury (ALI) or Chronic Obstructive Pulmonary Disease (COPD).

TABLE 18
List of IPF gene signature genes incorporated into Singscore

| | Indication | | Indication | | Indication | | Indication |
|---|---|---|---|---|---|---|---|
| Gene | in IPF | Gene | in IPF | Gene | in IPF | Gene | in IPF |
| AASS | down | LOXL3 | up | DOCK5 | down | RP11-2N1.2 | down |
| ABCA3 | down | LPCAT1 | down | DOCK9 | down | RP11- | up |
| | | | | | | 329B9.4 | |
| ABCB4 | up | LRIG3 | up | DOT1L | down | RP11- | up |
| | | | | | | 348N5.7 | |
| ABHD17C | down | LRP1 | up | DPT | up | RP11- | up |
| | | | | | | 403A21.1 | |
| ABHD5 | down | LRRC15 | up | DPYSL3 | up | RP11- | down |
| | | | | | | 432J24.5 | |
| AC007952.5 | down | LRRC17 | up | DRC3 | up | RP11- | up |
| | | | | | | 498C9.13 | |
| AC009237.16 | down | LRRC27 | up | DRP2 | up | RP1-152L7.5 | up |
| AC009237.17 | down | LRRC4C | up | DSCAM | down | RP11-54O7.1 | up |
| AC009238.7 | down | LRRN4 | down | DSEL | down | RP11- | up |
| | | | | | | 54O7.16 | |
| AC009238.8 | down | LSM6 | down | DTX1 | up | RP11- | up |
| | | | | | | 567M16.1 | |
| AC073130.3 | down | LSS | down | DUSP6 | down | RP11- | down |
| | | | | | | 61J19.5 | |
| AC124789.1 | down | LTBP1 | up | DUXAP8 | up | RP11- | up |
| | | | | | | 624L4.1 | |
| ACADS | down | LTBP2 | up | DUXAP9 | up | RP11- | down |
| | | | | | | 63G10.4 | |
| ACAT1 | down | LTBP3 | up | E2F1 | down | RP11- | up |
| | | | | | | 867G23.10 | |
| ACER3 | down | LUM | up | EAF2 | up | RP11- | down |
| | | | | | | 867O8.11 | |
| ACKR4 | down | LUZP2 | up | EBPL | down | RP11- | up |
| | | | | | | 893F2.5 | |
| ACOX2 | up | MACIR | down | ECHDC3 | down | RP11-92C4.6 | up |
| ACP3 | down | MAGED1 | up | ECM1 | up | RP11- | down |
| | | | | | | 93B14.10 | |
| ACTA2 | up | MAGED2 | up | ECM2 | up | RP11- | down |
| | | | | | | 96C23.10 | |
| ACTG2 | up | MAGED4 | up | EEF1AKNMT | down | RP11- | down |
| | | | | | | 96C23.5 | |
| ACTN1 | up | MAGED4B | up | EFCAB12 | up | RP11- | up |
| | | | | | | 999E24.3 | |
| ACVR1 | up | MAGEL2 | up | EFCAB6 | up | RP1- | up |
| | | | | | | 228H13.5 | |
| ACVRL1 | down | MAOA | down | EFHB | up | RP1- | down |
| | | | | | | 267D11.6 | |
| ADAM12 | up | MAP3K4-AS1 | up | EFHC1 | up | RP1-37C10.7 | up |
| ADAM19 | up | MAP3K6 | down | EFHC2 | up | RP3- | down |
| | | | | | | 331H24.6 | |
| ADAMTS10 | up | MAP3K7CL | up | EFL1 | down | RP3- | up |
| | | | | | | 337O18.9 | |
| ADAMTS16 | up | MAP4K2 | down | EFNA4 | up | RP3- | down |
| | | | | | | 342P20.2 | |
| ADAMTS6 | up | MAP6 | up | EFNB3 | up | RP3- | down |
| | | | | | | 403A15.5 | |
| ADCY5 | up | MAPK13 | down | ELAPOR1 | up | RP4-565E6.1 | up |
| ADCY8 | down | MARCKSL1 | up | EME2 | down | RP4-622L5.7 | up |
| ADGRA2 | up | MBP | down | EMILIN1 | up | RP5- | up |
| | | | | | | 1054A22.4 | |
| ADI1 | down | MCAM | up | EMP2 | down | RPS6KA1 | down |
| ADORA1 | up | MCCC1 | down | ENAH | up | RPS6KA2 | down |
| ADPRH | down | MCFD2 | down | ENOSF1 | down | RTN4 | down |
| ADRA1B | down | MDFI | up | ENTPD1 | up | RTTN | down |
| ADRB2 | down | MDK | up | ENTPD1-AS1 | up | RUNX1 | up |
| ADSS1 | up | MEG3 | up | ENTPD7 | up | S100A3 | down |
| AEBP1 | up | MEG8 | up | EPDR1 | down | SALL4 | up |
| AFAP1L1 | down | MEGF8 | up | EPHB2 | up | SAMD11 | up |
| AFDN | down | MEIS3 | up | EPHB3 | up | SASH1 | down |
| AGPAT2 | down | MEOX1 | up | ERBB3 | down | SCG2 | up |
| AGPAT3 | down | MEX3A | up | ERGIC3 | up | SCG5 | up |
| AGT | up | MFAP2 | up | ERRFI1 | down | SCML1 | down |
| AHNAK | down | MFAP4 | up | ESM1 | down | SCPEP1 | up |
| AKAP1 | down | MFSD13A | down | ETV6 | up | SCRG1 | up |
| AKNA | up | MFSD2A | down | EVA1A | down | SCUBE3 | up |
| ALCAM | down | MGLL | down | EXTL1 | up | SDC3 | up |
| ALDH18A1 | up | MGP | up | EYA2 | up | SEC23A | up |
| ALDH1L2 | up | MGST1 | down | FABP5 | down | SEC24D | up |
| ALKAL1 | up | MIDEAS | down | FAH | down | SEC31A | up |
| ALPK2 | up | MINAR1 | up | FAIM2 | up | SEL1L3 | up |
| ALS2CL | down | MIR100HG | up | FAM110A | down | SELENOM | up |
| AMD1 | down | MIR503HG | up | FAM111A | down | SEMA3C | up |
| AMOTL2 | down | MIR99AHG | up | FAM118A | up | SEMA3E | down |
| ANGPTL2 | up | MLLT11 | up | FAM131B | up | SEPTIN6 | up |
| ANK2 | up | MLPH | down | FAM133A | up | SERPINE2 | up |
| ANKH | up | MLX | down | FAM13C | up | SERPINF1 | up |
| ANKRD20A5P | up | MME | down | FAM160A1 | down | SERPINI1 | up |
| ANKRD29 | down | MMP10 | up | FAM167A | down | SESN3 | up |
| ANKRD33B | down | MMP11 | up | FAM171B | up | SFRP4 | up |
| ANO4 | up | MMP13 | up | FAM198B- | up | SFTA1P | down |
| | | | | AS1 | | | |
| ANTXR1 | up | MMP2 | up | FAM227A | up | SGCA | up |
| ANXA3 | down | MMP21 | up | FAM227B | up | SGCD | up |
| AOPEP | up | MMP3 | up | FAM229B | up | SGO1 | down |
| APLP1 | up | MORC4 | up | FAM234B | down | SGPL1 | up |
| APOL3 | down | MPP2 | up | FAM66D | up | SH2D5 | down |
| ARAP3 | down | MPZL1 | up | FAM98A | up | SH3BGR | up |
| ARF4 | up | MRAS | up | FANK1 | up | SH3PXD2A | up |
| ARHGAP29 | down | MRC2 | up | FAP | up | SH3PXD2B | up |
| ARHGAP6 | down | MROH8 | up | FASN | down | SH3RF1 | down |
| ARHGEF25 | up | MRPL14 | down | FBLIM1 | up | SH3RF3 | up |
| ARHGEF26 | down | MSC | up | FBLN1 | up | SH3RF3- | up |
| | | | | | | AS1 | |
| ARL6IP6 | down | MSC-AS1 | up | FBLN2 | up | SHANK2 | down |
| ARMCX2 | up | MT1F | up | FBN1 | up | SHISAL1 | up |
| ARMH4 | up | MTHFD1L | up | FBXL13 | up | SHMT1 | down |
| ASIC1 | up | MTMR12 | down | FBXL22 | up | SHROOM1 | down |
| ASPN | up | MXRA8 | up | FBXO32 | up | SIGIRR | down |
| ASS1 | up | MYH11 | up | FEM1C | down | SLC14A1 | down |
| ASTN1 | up | MYO1C | down | FGD4 | down | SLC16A1 | up |
| ASTN2 | up | MYOCD | up | FGF14 | up | SLC16A12 | down |
| ATAT1 | up | MYOM1 | up | FGF18 | up | SLC16A2 | up |
| ATG13 | up | MYOSLID | up | FH | down | SLC18B1 | up |
| ATP11A | down | NAALADL2 | up | FHL2 | up | SLC1A4 | up |
| ATP1A2 | up | NADK | down | FILIP1L | up | SLC22A17 | up |
| ATP2A3 | up | NAGS | down | FKBP10 | up | SLC25A24 | down |
| AZIN2 | up | NAP1L3 | up | FKBP11 | up | SLC25A25 | down |
| B3GALNT1 | down | NBAT1 | up | FKBP14 | up | SLC25A4 | down |
| B3GALNT2 | up | NBEA | up | FKBP4 | down | SLC25A5 | down |
| B4GALT1 | up | NBEAL2 | down | FKBP7 | up | SLC29A3 | up |
| B4GALT4 | up | NCAPH2 | down | FLII | down | SLC2A1 | up |
| BACE2 | up | NCKAP5 | down | FLRT2 | up | SLC2A10 | up |
| BAIAP2 | down | NCKAP5-AS2 | down | FLRT3 | down | SLC35F2 | up |
| BBOF1 | up | NDUFA6-DT | up | FLT1 | down | SLC38A4 | up |
| BCAR3 | down | NEDD4 | down | FLVCR2 | down | SLC44A2 | down |
| BCAT2 | down | NEDD4L | down | FMO3 | up | SLC44A3- | up |
| | | | | | | AS1 | |
| BCHE | up | NEK11 | up | FMOD | up | SLC46A3 | up |
| BCL2L1 | down | NEMP1 | down | FN1 | up | SLC51B | down |
| BCL9 | up | NFATC4 | up | FN3K | down | SLC66A1L | down |
| BCO2 | up | NFKBIA | down | FNDC5 | up | SLC7A5 | up |
| BGN | up | NHSL1 | down | FOLR3 | down | SLCO4A1 | down |
| BICC1 | up | NIBAN2 | down | FRK | up | SLIT3 | up |
| BLNK | up | NIPA1 | down | FRMD5 | up | SLITRK2 | down |
| BLVRB | down | NIPSNAP3B | up | FRMD6 | up | SLITRK6 | up |
| BMERB1 | up | NLGN2 | up | FRZB | up | SMAGP | down |
| BMP2 | down | NNMT | up | FSTL1 | up | SMC4 | down |
| BMP4 | up | NOTCH1 | down | FUT8 | up | SMIM10L2A | up |
| BMPER | down | NOTCH3 | up | FZD5 | down | SMIM29 | down |
| BMPR1B | up | NPAS2 | up | FZD7 | up | SMIM43 | up |
| BNC2 | up | NPC1 | down | GABRB3 | up | SMO | up |
| BNIP3 | down | NR2F1-AS1 | up | GAL | up | SMOX | up |
| BOC | up | NR3C2 | down | GALE | down | SMURF2 | down |
| BRI3 | down | NRGN | down | GALK1 | down | SNAI2 | up |
| BRINP3 | up | NRP2 | up | GALNT1 | up | SNCAIP | up |
| BTBD6 | down | NTHL1 | down | GALNT16 | up | SNED1 | up |
| C10orf95-AS1 | down | NTNG1 | down | GALNT3 | down | SNRPN | up |
| C13orf46 | down | NUDCD1 | down | GAN | up | SORCS2 | up |
| C16orf71 | up | NUDT15 | down | GAP43 | up | SOX4 | up |
| C1GALT1 | down | NUDT16L2P | down | GAS6-AS1 | up | SPARC | up |
| C1orf115 | down | NUP58 | down | GAS7 | up | SPC24 | down |
| C1orf122 | up | NYNRIN | up | GASK1B | up | SPC25 | down |
| C1orf21 | down | OCLN | down | GATA2 | down | SPECC1 | up |
| C1orf54 | up | OGDH | down | GBE1 | down | SPON1 | up |
| C1QTNF3 | up | OGN | up | GCAT | down | SPRY2 | down |
| C1QTNF6 | up | OLFM2 | up | GCDH | down | SPRY4 | down |
| C1R | up | OLFML2B | up | GCNT2 | down | SPRY4-AS1 | down |
| C1RL | up | OLFML3 | up | GDF6 | up | SPSB1 | up |
| C1S | up | OMD | up | GDNF | up | SPTBN1 | down |
| C20orf27 | down | OMG | up | GEMIN4 | down | SPTLC3 | down |
| C20orf85 | up | OSBPL10 | up | GFUS | up | SQOR | down |
| C4A | up | OSGIN1 | down | GIMAP2 | down | SRGAP3 | up |
| C4B | up | OSR2 | up | GIT1 | down | SRRM3 | up |
| C4orf46 | down | OSTF1 | down | GJC2 | down | SSC5D | up |
| C5orf66 | up | P2RY1 | down | GLI1 | up | SSPN | up |
| C7 | up | P3H1 | up | GLI2 | up | SSR4 | up |
| CABCOCO1 | up | P3H2 | down | GLIS3 | up | SSTR1 | down |
| CACNA1C | up | P3H3 | up | GLT8D1 | up | ST8SIA2 | up |
| CALB2 | up | P3H4 | up | GLT8D2 | up | STAC | down |
| CALD1 | up | P4HA3 | up | GOLGA2 | up | STARD3NL | down |
| CAMK1D | up | PAK3 | up | GOLM1 | up | STARD7 | down |
| CAMK2D | down | PAK4 | down | GPAT3 | down | STARD8 | down |
| CAP2 | up | PALB2 | down | GPC2 | up | STBD1 | down |
| CAPN2 | down | PALM2AKAP2 | down | GPD1L | down | STEAP3- | up |
| | | | | | | AS1 | |
| CAPS | up | PAMR1 | up | GPD2 | down | STK38L | up |
| CARD10 | down | PAPPA | up | GPER1 | down | STRADB | down |
| CASC15 | up | PAPPA2 | up | GPN3 | down | STX11 | down |
| CASP4LP | down | PAPPA-AS1 | up | GPR153 | up | STX3 | down |
| CASTOR3 | up | PAQR5 | down | GPR155 | up | SUFU | up |
| CASZ1 | down | PARD6G | up | GPR160 | down | SUGCT | up |
| CAV1 | down | PARP12 | down | GPR173 | up | SULF2 | up |
| CAVIN2 | down | PAX6 | up | GPR183 | up | SUN2 | down |
| CBR1 | down | PBLD | up | GPR78 | up | SVIP | down |
| CC2D1B | down | PBXIP1 | up | GPRACR | up | SYNDIG1 | up |
| CC2D2B | up | PCAT6 | up | GPRC5A | down | SYNPO2L | down |
| CCBE1 | down | PCDH19 | up | GPX7 | up | SYT12 | up |
| CCDC144NL- | up | PCDHB12 | up | GPX8 | up | SYTL4 | down |
| AS1 | | | | | | | |
| CCDC170 | up | PCDHB14 | up | GRIA3 | up | TACC2 | down |
| CCDC180 | up | PCDHB2 | up | GRK5 | down | TACR1 | up |
| CCDC40 | up | PCDHB7 | up | GSAP | down | TAGLN | up |
| CCDC68 | down | PCID2 | down | GSEC | up | TAOK3 | down |
| CCDC74A | up | PCOLCE | up | GSN | up | TBC1D4 | down |
| CCDC8 | up | PCSK1 | up | GXYLT2 | up | TBRG4 | down |
| CCDC80 | up | PCYOX1L | up | HACD1 | down | TBX2-AS1 | down |
| CCDC85C | down | PCYT2 | down | HAGH | down | TCF21 | down |
| CCK | down | PDCD4 | up | HAPLN3 | up | TDRD7 | down |
| CCND2 | up | PDE12 | down | HAUS4 | down | TEAD4 | down |
| CCND3 | down | PDE4DIPP2 | down | HCG27 | down | TEC | down |
| CD24 | up | PDGFRB | up | HEPH | up | TENM3 | up |
| CD274 | down | PDIA3 | up | HHAT | up | TENM4 | up |
| CD36 | down | PDIA4 | up | HIC1 | up | TENT5B | down |
| CD47 | down | PDLIM3 | up | HIF3A | down | TEX9 | up |
| CD55 | down | PDLIM4 | up | HILPDA | up | TF | up |
| CDAN1 | down | PDLIM7 | up | HIRIP3 | down | TFPI | down |
| CDCA7L | down | PDP2 | down | HK2 | down | TGFB3 | up |
| CDH11 | up | PDZRN3 | up | HLA-E | down | TGFBI | up |
| CDH2 | up | PEAR1 | down | HMCN1 | up | TGFBR3 | down |
| CDH6 | up | PECR | down | HMSD | down | THBS2 | up |
| CDHR1 | up | PEG10 | down | HNRNPF | down | THBS3 | up |
| CDKL1 | down | PGAM5 | down | HOPX | down | TIMP1 | up |
| CDKL2 | down | PGAP6 | down | HOXA3 | up | TJP2 | down |
| CDKN1A | up | PGBD5 | up | HPCAL1 | down | TLR3 | down |
| CDKN2AIPNL | down | PGM2L1 | up | HS3ST3A1 | up | TM4SF4 | down |
| CDKN2D | down | PGM5 | up | HSBP1L1 | down | TMBIM1 | down |
| CELF2 | down | PHLPP1 | down | HSDL2 | down | TMED3 | up |
| CELSR2 | up | PI4K2B | down | HSP90B1 | up | TMEM106C | down |
| CENPX | down | PIEZO1 | down | HSPB7 | up | TMEM117 | up |
| CEP72 | down | PITPNM2 | down | HTR2B | up | TMEM119 | up |
| CEP85 | down | PITRM1 | down | HTRA1 | up | TMEM132A | up |
| CERCAM | up | PKDCC | down | HTRA3 | up | TMEM182 | up |
| CERS2 | down | PKN1 | down | HYAL2 | down | TMEM190 | up |
| CFAP69 | up | PKN3 | down | HYDIN2 | up | TMEM192 | down |
| CFH | up | PKP1 | up | ICOSLG | up | TMEM200B | down |
| CFL2 | down | PLAAT3 | down | IDH2 | up | TMEM231 | up |
| CGN | down | PLCB4 | up | IER2 | down | TMEM245 | down |
| CH507-9B2.1 | up | PLEKHA6 | up | IER5L | up | TMEM263 | up |
| CHAC2 | down | PLEKHJ1 | down | IFIT3 | down | TMEM45A | up |
| CHD3 | up | PLEKHM1 | down | IFIT5 | down | TMEM53 | down |
| CHN1 | up | PLIN2 | down | IFITM10 | up | TMEM59L | up |
| CHPF | up | PLN | up | IFT27 | up | TMEM62 | down |
| CHPT1 | down | PLOD1 | up | IFT43 | up | TMPO | down |
| CHRAC1 | down | PLOD2 | up | IGDCC4 | up | TMSB15B | up |
| CHRDL2 | up | PLP1 | up | IGF1 | up | TMTC1 | down |
| CHST6 | up | PLPP4 | up | IGF2BP2 | down | TNC | up |
| CHSY1 | up | PLPP5 | up | IGFBP3 | up | TNFRSF21 | up |
| CILP | up | PLTP | up | IGFBP7 | up | TNFSF18 | up |
| CILP2 | up | PMEPA1 | up | IL11 | up | TNFSF4 | up |
| CIT | down | PMM1 | down | IL15RA | down | TNNT1 | down |
| CITED2 | down | PNMA8A | up | IL17RD | up | TOM1L1 | down |
| CKAP4 | up | PNMA8B | up | IL17RE | down | TOR4A | down |
| CLCA2 | up | PODN | up | IL18 | down | TOX | down |
| CLDN12 | down | PODNL1 | up | IL6R | down | TOX2 | down |
| CLDN4 | down | PODXL | down | IMP3 | down | TP53 | up |
| CLEC14A | down | POGLUT2 | up | IMPA1 | down | TP53INP1 | up |
| CLEC3B | down | POLA2 | down | INF2 | down | TP53TG1 | up |
| CLIP3 | up | POLE | down | INMT | down | TPRN | down |
| CLMP | up | POPDC3 | down | INSYN1 | up | TPST1 | up |
| CLPP | down | POSTN | up | INSYN2A | up | TPST2 | down |
| CLSTN2 | up | POU2F2 | up | INTS6L | up | TRAF5 | up |
| CLSTN3 | up | PPA1 | down | IPO5 | down | TRAV30 | up |
| CLU | up | PPARG | down | IQCK | up | TRHDE-AS1 | down |
| CNPY4 | up | PPFIBP1 | down | IRAK3 | down | TRIM25 | down |
| CNTROB | down | PPIB | up | ISLR | up | TRIM32 | up |
| COL10A1 | up | PPIC | up | ITGA11 | up | TRIM58 | down |
| COL14A1 | up | PPIC-AS1 | up | ITGA3 | down | TRIM62 | up |
| COL15A1 | up | PPL | down | ITGA9 | up | TRNP1 | down |
| COL16A1 | up | PPM1F | down | ITGAV | up | TRO | up |
| COL1A1 | up | PPP1R12B | up | ITGB3 | up | TRPS1 | up |
| COL1A2 | up | PPP1R15A | down | ITGB5 | up | TRPV4 | up |
| COL24A1 | up | PPP2R5A | down | ITGBL1 | up | TSKU | up |
| COL27A1 | up | PRDM1 | up | ITM2C | up | TSPAN11 | up |
| COL3A1 | up | PRDM6 | up | ITPK1 | down | TSPAN2 | up |
| COL4A2-AS2 | up | PRDX4 | up | ITPR3 | down | TSPAN4 | down |
| COL5A1 | up | PREX1 | down | ITPRID2 | down | TSPAN6 | up |
| COL5A2 | up | PRG4 | up | JAK3 | up | TTC3 | up |
| COL6A1 | up | PRKACB | up | JAZF1 | up | TTC39A | down |
| COL6A2 | up | PRKAR2B | down | KAT2B | down | TTC9 | up |
| COL6A3 | up | PRKCE | down | KCNA2 | up | TTLL1 | up |
| COL7A1 | up | PRKCQ-AS1 | down | KCND1 | up | TTLL11 | up |
| COL9A2 | up | PRKG2 | down | KCND3 | up | TTLL12 | down |
| COLQ | up | PRLR | down | KCNH1 | up | TTN | down |
| COMP | up | PROSER2 | down | KCNN4 | up | TTYH3 | up |
| COPZ2 | up | PRRG4 | down | KCTD11 | up | TUB | up |
| CPE | up | PRUNE2 | up | KDELR2 | up | TUBGCP3 | down |
| CPEB2 | down | PSD2 | up | KDM5B | up | TWIST1 | up |
| CPNE3 | down | PSD3 | up | KHDRBS3 | down | TXLNB | up |
| CPNE8 | down | PTCHD4 | up | KIAA1755 | up | TXNRD1 | down |
| CPQ | up | PTGFRN | up | KIF17 | down | TXNRD2 | down |
| CPZ | up | PTGIS | up | KIF26B | up | UCHL1 | up |
| CRACD | up | PTHLH | up | KIF3C | up | UNC13B | down |
| CREB3L2 | up | PTK7 | up | KIF5A | up | UNC5C | up |
| CRLF1 | up | PTPRQ | down | KIF9-AS1 | up | USP1 | down |
| CROT | up | PVR | down | KIFC3 | down | USP13 | down |
| CS | down | PYCARD | down | KLF15 | down | USP31 | down |
| CSF2 | down | PYCR1 | up | KLF6 | down | USP53 | down |
| CSGALNACT1 | up | QDPR | down | KLF9 | down | USP54 | down |
| CSMD2 | up | QPCT | up | KLHL4 | up | UTP18 | down |
| CST1 | up | RAB11FIP1 | down | KNSTRN | down | UTRN | down |
| CTC-308K20.1 | down | RAB17 | down | KSR1 | up | VASH2 | up |
| CTD-2003C8.2 | down | RAB20 | down | LAMA3 | down | VCAM1 | up |
| CTHRC1 | up | RAB30 | up | LCA5L | up | VCAN | up |
| CTNNAL1 | down | RAB32 | down | LDB3 | up | VCAN-AS1 | up |
| CTSH | down | RAB3D | down | LDLR | down | VEPH1 | down |
| CTSO | up | RABL2A | up | LDLRAD4 | up | VMP1 | up |
| CUL7 | up | RAMP1 | up | LEF1 | up | VSIG10 | down |
| CXXC5 | up | RAP1GAP2 | down | LETM2 | down | VSIR | down |
| CYB5A | down | RAP2B | up | LGMN | up | VWA1 | up |
| CYC1 | down | RAPGEF4 | down | LGR4 | up | VWCE | up |
| CYSTM1 | down | RASA4 | up | LHPP | up | WDR5 | down |
| DACH2 | up | RASA4B | up | LIMD1 | down | WHRN | up |
| DACT1 | up | RASD2 | up | LINC00472 | down | WIPI1 | up |
| DACT3 | up | RASGRP3 | up | LINC00475 | up | WNT11 | up |
| DACT3-AS1 | up | RASIP1 | down | LINC00513 | down | WNT3 | down |
| DAGLB | down | RASL11B | up | LINC00519 | up | WWC1 | down |
| DARS2 | down | RBP1 | up | LINC00535 | up | WWC3 | down |
| DCLRE1A | down | RCAN2 | up | LINC00578 | up | XBP1 | up |
| DCXR | down | RCC2 | up | LINC00632 | up | Xxyac- | up |
| | | | | | | YX65C7_A.2 | |
| DDIT4 | up | RCN3 | up | LINC00643 | up | XXYLT1 | up |
| DDX28 | down | RCOR2 | up | LINC01013 | up | ZBTB42 | down |
| DELEC1 | up | RERG | up | LINC01133 | up | ZC3H12C | down |
| DENND2B | up | RGMB | down | LINC01138 | up | ZDHHC12 | down |
| DENND3 | down | RGS2 | up | LINC01224 | down | ZDHHC14 | down |
| DERA | down | RHBDF1 | down | LINC01273 | down | ZDHHC7 | down |
| DERL3 | up | RHOF | down | LINC01503 | up | ZFP69B | up |
| DGKA | up | RIMS2 | up | LINC01614 | up | ZFPM2 | up |
| DGKE | down | RIPOR1 | down | LINC01615 | up | ZKSCAN7 | up |
| DGKI | up | RN7SL689P | up | LINC01711 | up | ZMAT3 | up |
| DIAPH3 | down | RNF150 | up | LINC01943 | up | ZNF154 | up |
| DLL4 | down | RNH1 | down | LINC02185 | down | ZNF185 | down |
| DMD | up | ROBO1 | up | LINC02544 | up | ZNF423 | up |
| DMGDH | up | ROBO2 | up | LINC02593 | up | ZNF436 | up |
| DNAH1 | up | ROBO4 | down | LINC02606 | up | ZNF469 | up |
| DNAJB13 | up | ROR2 | up | LINC02694 | up | ZNF521 | up |
| DNAJB5 | up | RP11- | up | LINC02731 | up | ZNF561-AS1 | up |
| | | 1151B14.4 | | | | | |
| DNAJC12 | up | RP11-119F7.5 | up | LIPH | down | ZNF711 | up |
| DNAJC22 | up | RP11- | up | LMO4 | up | ZNF726P1 | down |
| | | 125O18.1 | | | | | |
| DNHD1 | down | RP11-145A3.1 | up | LMOD1 | up | ZNF792 | down |
| DNM1 | up | RP11-167N4.2 | up | LNCTAM34A | up | ZNF823 | down |
| DNMT3A | up | RP11-212I21.3 | up | LOX | up | ZNF827 | up |
| DNPEP | down | RP11-229O3.1 | up | LOXL2 | up | ZNF846 | up |

In some aspects, the modulator further modulates RNA expression level, protein expression level, or both, of one or more genes from Table 18. In some aspects, the modulator reduces RNA expression level, protein expression level, or both, of one or more genes from Table 18. In some aspects, the modulator modulates RNA expression level, protein expression level, or both, of subsets of genes associated with autophagy, ubiquitin, cilium and flagellum assembly and function, extracellular matrix (e.g., collagen) synthesis, metabolism, deposition, and organization, cell organization, genome regulation, or ncRNA and mRNA regulation.

In some aspects, the modulator reduces RNA expression level, protein expression level, or both, of one or more fibrotic genes. In some aspects, the one or more fibrotic genes comprise COL3A1, COL1A1, ACTA2, POSTN, FN1, FAP, CTHRC1, or combination thereof. In some aspects, inhibition of expression or activity of the lncRNA modulates expression of the one or more fibrotic marker genes (e.g., ACTA2, COL1A1, COL3A1, FAP, FN1, POSTN, CTHRC1, THBS1, THBS2, etc.). In some aspects, inhibition of expression or activity of the lncRNA decreases expression of the one or more fibrotic marker genes (e.g., ACTA2, COL1A1, COL3A1, FAP, FN1, POSTN, CTHRC1, THBS1, THBS2, etc.). In some aspects, inhibition of expression or activity of the lncRNA decreases expression of the one or more fibrotic marker genes (e.g., ACTA2, COL1A1, COL3A1, FAP, FN1, POSTN, CTHRC1, THBS1, THBS2, etc.) at least 5%, 10%, 15%, 20%, 30%, 40%, 50%, 60%, 70%, 80%, or 90% or more. In some aspects, inhibition of expression or activity of the lncRNA decreases expression of the one or more fibrotic marker genes (e.g., ACTA2, COL1A1, COL3A1, FAP, FN1, POSTN, CTHRC1, THBS1, THBS2, etc.) at least 5%, 10%, 15%, 20%, 30%, 40%, 50%, 60%, 70%, 80%, or 90% or more from the increased expression level of the one or more fibrotic marker genes in the myofibroblast, induced myofibroblast, a cell affected by pulmonary fibrosis, e.g., IPF, a cell affected by IPF, a tissue affected by pulmonary fibrosis, e.g., IPF, or a tissue affected by IPF. In some aspects, inhibition of expression or activity of the lncRNA modulates a fibrotic status of a tissue affected by pulmonary fibrosis, e.g., IPF, or a tissue affected by IPF. In some aspects, inhibition of expression or activity of the lncRNA slows down the progress of a fibrotic status of a tissue affected by pulmonary fibrosis, e.g., IPF, or a tissue affected by IPF. In some aspects, inhibition of expression or activity of the lncRNA reduces or prevents progress of a fibrotic status of a tissue affected by pulmonary fibrosis, e.g., IPF, or a tissue affected by IPF. In some aspects, inhibition of expression or activity of the lncRNA reverses progress of a fibrotic status of a tissue affected by pulmonary fibrosis, e.g., IPF, or a tissue affected by IPF.

In the three-dimensional (3D) conformation of the genome, a topologically associating domain (TAD) characterizes a genomic region where DNA sequences within a TAD physically interact with each other more often than with sequences located outside the TAD. Boundaries of both sides of these domains or TAD boundaries contribute to regulation of gene expression by restricting interactions of cis-regulatory sequence to their target genes. In some instances, TAD boundaries are enriched with transcription factors, e.g., CTCF binding factor, enhancers, protein complex, e.g., cohesion, thereby impacting transcription of genes within the same TAD. In some instances, modulation of gene expression via 3D chromatic structure can affect physiological and pathological functions. In some aspects, the lncRNA disclosed herein shares a TAD with one or more genes located upstream or downstream of the lncRNA. In some aspects, the lncRNA shares a TAD with one or more genes located upstream of the lncRNA. In some aspects, the lncRNA shares a TAD with one or more genes located downstream of the lncRNA. In some aspects, the modulator described herein modulates transcription of the one or more genes. In some aspects, the modulator decreases the transcription of the one or more genes. In some aspects, the modulator increases the transcription of the one or more genes. In some aspects, the one or more genes comprises MX1, MX2, OAS1, OAS2, and OAS3.

Co-expression network analysis of gene expression can be utilized to investigate correlation patterns of genes among samples. In some instances, the co-expression network analysis, e.g., weighted gene co-expression network analysis, is used for identifying modules or clusters of highly correlated genes. In some instances, the highly correlated genes detected in this co-expression module can be utilized as candidate markers or therapeutic targets. In some aspects, the modulator modulates RNA expression level, protein expression level, or both of one or more genes detected in co-expression module. In some aspects, the one or more genes are MX1, MX2, thrombospondin 1 (THBS1), thrombospondin 2 (THBS2), or combination thereof. In some aspects, the one or more genes are B9D1, IQCG, LRRC23, TMEM231, SPACA9, CCDC65, ARHGAP39, C9orf116, CFAP300, RAB36, or a combination thereof. In some aspects, the one or more genes are CCDC60, FAM81B, TCTE1, MDHIB, RSPH9, PACRG, C9orf116, ODAD4, TMEM231, or a combination thereof. In some aspects, the one or more genes are NTN1, TMEM45A, GOLM1, SERPINB5, FAT2, SPTBN2, or a combination thereof. In some aspects, the one or more genes are CERCAM, COL3A1, COL1A1, COL5A2, COL1A2, C1S, ROR2, EPHB2, COL15A1, CTHRC1, or a combination thereof.

In some aspects, the modulator restores a cytokine level that is similar to a reference cytokine level obtained from a population of healthy subjects. In some aspects, the cytokine comprises IL-9, IL-1α, MIP-1α (CCL3), G-CSF, IL-2, IL-7, or a combination thereof.

In some aspects, the modulator modulates expression of lncRNA that is associated with cell type transition. The modulator inhibits epithelial to mesenchymal transition (EMT) of pulmonary cells. In some aspects, the pulmonary cells comprise pulmonary fibroblasts, pulmonary myofibroblasts, or epithelial cells. In some aspects, the pulmonary fibroblasts, the pulmonary myofibroblasts, or the epithelial cells are in the middle to lower airway of the lung. In some aspects, the modulator of the lncRNA inhibits or prevents the morphological or physiological changes of a fibroblast to myofibroblast or epithelial cell to myofibroblast in a tissue affected by pulmonary fibrosis, e.g., IPF. In some aspects, the modulator of the lncRNA inhibits or prevents the morphological or physiological changes of a fibroblast to myofibroblast or epithelial cell to myofibroblast in a tissue affected by IPF.

In one aspect, the modulator targets epigenomic features that allow disruption of cis- and trans-functional mechanisms of the lncRNA. In some aspects, the modulator is generated based on epigenomic, epigenetic, genetic, and transcriptional signal data, which collectively define the target epigenomic features as target regions of interest. In some aspects, the target epigenomic features comprises one or more epigenomic signals. In some aspects, the one or more epigenomic signals comprises histone modifications, DNA accessibility, DNA binding of relevant transcription factor e.g., CTCF, chromatin conformation, or combination thereof. In some aspects, the histone modifications are profiled with Cut&Run, ChipSeq, or other similar methodologies known in the art. In some aspects, the histone modifications help identifying active promoter, enhancer, and transcriptionally active regions that are labelled as target regions of interest. In some aspects, the histone modifications comprise H3K27ac, H3K4me1 and H3K4me3. In some aspects, the DNA accessibility is profiled with ATAC-seq both in bulk and single cell/single nuclei, or other similar methodologies known in the art. In some aspects, the DNA binding of relevant transcription factor is profiled using Cut&Run and ChipSeq, in silico analyses of motif analysis, or other similar methodologies known in the art. In some aspects, the DNA binding of relevant transcription factor comprises information obtained from Transcription Factor Binding Sites (TFBS). In some aspects, the chromatin conformation comprises information obtained from 4C, HiC, MicroC profiling, In Silico Machine Learning approaches based on genomic features, or other similar methodologies known in the art. In some aspects, the genetic signals comprise relevant genetics variants, e.g., single nucleotide polymorphism SNPs, indels, or copy number variations (CNVs). In some aspects, the transcriptional signals are obtained from expression profiling such as RNASeq, ProSeq, ProCap, long read sequencing, e.g., PacBio IsoSeq or Oxford Nanopore. In some aspects, in PUFIN2, chr15: 39,113,871-39,226,735 is the approximate location of a super enhancer that overlaps with PUFIN2. In some aspects, the genomic loci of chr15: 39,426,796-39,497,305 comprise several isoforms of PUFIN2 with 4 open chromatin binding peaks identified through snATACseq that can be transcribed into lncRNAs, and the modulator is generated to target this region of the lncRNAs.

In another aspect, the present disclosure provides a modulator that can modulate the RNA expression or function of the lncRNA. In some aspects, the modulator prevents RNA expression of the lncRNA. In some aspects, the modulator inhibits function of the lncRNA. In some aspects, the modulator comprises nucleic acid molecule, oligonucleotide, polynucleotide, small molecule, or biological molecule. In some instances, the modulator targets the genetics variants, e.g., SNP, thereby affecting the expression and/or activity of lncRNAs described herein. In some instances, the modulator targets the genetics variants, e.g., SNP, thereby affecting IPF pathology. In some instances, the genetics variants, e.g., SNP, are shown in any one of Tables 46-69.

### Oligonucleotide

In some aspects, the modulator disclosed herein is an oligonucleotide or polynucleotide. In some aspects, the oligonucleotide is a synthetic or artificial oligonucleotide. In some aspects, the oligonucleotide is a single-stranded nucleic acid molecule. In some aspects, the modulator comprises a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), or an enzymatically-prepared siRNA (esiRNA) or a precursor thereof.

In some aspects, the modulator is the oligonucleotide. In some aspects, the modulator is a synthetic or artificial oligonucleotide. In some aspects, the oligonucleotide disclosed herein is about 10-50 nucleotides long. In some instances, the oligonucleotide disclosed herein is about 10-40 nucleotides long. In some instances, the oligonucleotide disclosed herein is about 10-30, 10-28, 14-28, 14-25, 14-20, 15-25, or 18-25 nucleotides long. In some instances, the synthetic or artificial oligonucleotide is about 13-35 or about 16-20 nucleotides long. In some instances, the synthetic or artificial oligonucleotide is about 13-35 nucleotides long. In some instances, the synthetic or artificial oligonucleotide is about 16-20 nucleotides long.

In some aspects, the oligonucleotide disclosed herein is at least 10, 11, 12, 13, 14, 15, 16 nucleotides long. In some aspects, the oligonucleotide disclosed herein is at most 50, 40, 30, 20 nucleotides long. In some aspects, the oligonucleotide disclosed herein is about 15, 16, 17, 18, 19, 20 nucleotides long. In other aspects, the oligonucleotide disclosed herein is 16, 17, 18, 19, 20 nucleotides long.

In some aspects, the oligonucleotide is a double-stranded nucleic acid molecule comprising a sense strand and an antisense strand. In some aspects, the sense strand comprises a nucleic acid sequence at least 9, at least 10, at least 11, at least 12, at least 13, at least 14, or at least 15 consecutive nucleotides that are at least 80%, at least 85%, at least 90%, at least 95% identical to at least a portion of the lncRNAs disclosed herein. In some aspects, the antisense strand comprises a nucleic acid sequence comprising at least 9, at least 10, at least 11, at least 12, at least 13, at least 14, at least 15 consecutive nucleotides that are fully complementary or at least 80%, at least 85%, at least 90%, at least 95% complementary to at least a portion of the lncRNAs disclosed herein.

In some instances, the synthetic or artificial oligonucleotide comprises a nucleic acid sequence at least 80%, at least 90%, at least 95%, or at least 99% complementary to at least 10, at least 11, at least 12, at least 13, at least 14, or at least 15 contiguous nucleotides of the lncRNA described herein.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 1-2566 and 2580-3987. In some aspects, the modulator comprises a nucleic acid sequence comprising at least 80%, at least 85%, at least 90%, or at least 95% identical to a sequence selected from any one of SEQ ID Nos: 1-2566 and 2580-3987. In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 1-28,31-37, 378, 385, 387, 2010-2018, and 2564-2566. In some aspects, the modulator comprises a nucleic acid sequence comprising at least 80%, at least 85%, at least 90%, or at least 95% identical to a sequence selected from any one of SEQ ID Nos: 1-28,31-37, 378, 385, 387, 2010-2018, and 2564-2566. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 1-28,31-37, 378, 385, 387, 2010-2018, and 2564-2566. In some aspects, the modulator is a single stranded antisense oligonucleotide (ASO), and the ASO comprises at least 80%, at least 85%, at least 90%, at least 95% identical to any one of SEQ ID Nos: 1-28,31-37, 378, 385, 387, 2010-2018, and 2564-2566.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 2580-3987. In some aspects, the modulator comprises a nucleic acid sequence comprising at least 80%, at least 85%, at least 90%, or at least 95% identical to a sequence selected from any one of SEQ ID Nos: 2580-3987. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 2580-3987. In some aspects, the modulator is a single stranded antisense oligonucleotide (ASO), and the ASO comprises at least 80%, at least 85%, at least 90%, at least 95% identical to any one of SEQ ID Nos: 2580-3987.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 36-40 and 1406-1968. In some aspects, the modulator comprises a nucleic acid sequence comprising at least 80%, at least 85%, at least 90%, or at least 95% identical to a sequence selected from any one of SEQ ID Nos: 36-40 and 1406-1968. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 36-40 and 1406-1968. In some aspects, the modulator is a single stranded antisense oligonucleotide (ASO), and the ASO comprises at least 80%, at least 85%, at least 90%, at least 95% identical to any one of SEQ ID Nos: 36-40 and 1406-1968.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 1-9, 1969-2009, and 2564-2566. In some aspects, the modulator comprises a nucleic acid sequence comprising at least 80%, at least 85%, at least 90%, or at least 95% identical to a sequence selected from any one of SEQ ID Nos: 1-9, 1969-2009, and 2564-2566. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 1-9, 1969-2009, and 2564-2566. In some aspects, the modulator is a single stranded antisense oligonucleotide (ASO), and the ASO comprises at least 80%, at least 85%, at least 90%, at least 95% identical to any one of SEQ ID Nos: 1-9, 1969-2009, and 2564-2566.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 10-16 and 802-1405. In some aspects, the modulator comprises a nucleic acid sequence comprising at least 80%, at least 85%, at least 90%, or at least 95% identical to a sequence selected from any one of SEQ ID Nos: 10-16 and 802-1405. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 10-16 and 802-1405. In some aspects, the modulator is a single stranded antisense oligonucleotide (ASO), and the ASO comprises at least 80%, at least 85%, at least 90%, at least 95% identical to any one of SEQ ID Nos: 10-16 and 802-1405.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 2010-2013. In some aspects, the modulator comprises a nucleic acid sequence comprising at least 80%, at least 85%, at least 90%, or at least 95% identical to a sequence selected from any one of SEQ ID Nos: 2010-2013. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 2010-2013. In some aspects, the modulator is a single stranded antisense oligonucleotide (ASO), and the ASO comprises at least 80%, at least 85%, at least 90%, at least 95% identical to any one of SEQ ID Nos: 2010-2013.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 17-26 and 41-559. In some aspects, the modulator comprises a nucleic acid sequence comprising at least 80%, at least 85%, at least 90%, or at least 95% identical to a sequence selected from any one of SEQ ID Nos: 17-26 and 41-559. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 17-26 and 41-559. In some aspects, the modulator is a single stranded antisense oligonucleotide (ASO), and the ASO comprises at least 80%, at least 85%, at least 90%, at least 95% identical to any one of SEQ ID Nos: 17-26 and 41-559.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 27-35 and 560-801. In some aspects, the modulator comprises a nucleic acid sequence comprising at least 80%, at least 85%, at least 90%, or at least 95% identical to a sequence selected from any one of SEQ ID Nos: 27-35 and 560-801. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 27-35 and 560-801. In some aspects, the modulator is a single stranded antisense oligonucleotide (ASO), and the ASO comprises at least 80%, at least 85%, at least 90%, at least 95% identical to any one of SEQ ID Nos: 27-35 and 560-801.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 2014-2018. In some aspects, the modulator comprises a nucleic acid sequence comprising at least 80%, at least 85%, at least 90%, or at least 95% identical to a sequence selected from any one of SEQ ID Nos: 2014-2018. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 2014-2018. In some aspects, the modulator is a single stranded antisense oligonucleotide (ASO), and the ASO comprises at least 80%, at least 85%, at least 90%, at least 95% identical to any one of SEQ ID Nos: 2014-2018.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 2019-2073. In some aspects, the modulator comprises a nucleic acid sequence comprising at least 80%, at least 85%, at least 90%, or at least 95% identical to a sequence selected from any one of SEQ ID Nos: 2019-2073. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 2019-2073. In some aspects, the modulator is a single stranded antisense oligonucleotide (ASO), and the ASO comprises at least 80%, at least 85%, at least 90%, at least 95% identical to any one of SEQ ID Nos: 2019-2073.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 2074-2076. In some aspects, the modulator comprises a nucleic acid sequence comprising at least 80%, at least 85%, at least 90%, or at least 95% identical to a sequence selected from any one of SEQ ID Nos: 2074-2076. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 2074-2076. In some aspects, the modulator is a single stranded antisense oligonucleotide (ASO), and the ASO comprises at least 80%, at least 85%, at least 90%, at least 95% identical to any one of SEQ ID Nos: 2074-2076.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 2077-2310. In some aspects, the modulator comprises a nucleic acid sequence comprising at least 80%, at least 85%, at least 90%, or at least 95% identical to a sequence selected from any one of SEQ ID Nos: 2077-2310. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 2077-2310. In some aspects, the modulator is a single stranded antisense oligonucleotide (ASO), and the ASO comprises at least 80%, at least 85%, at least 90%, at least 95% identical to any one of SEQ ID Nos: 2077-2310.

In some aspects, the modulator comprises a nucleic acid molecule comprising at least 10, at least 11, at least 12, or at least 13 consecutive nucleotides with no more than 1, no more than 2, or no more than 3 mismatches from any one of SEQ ID Nos: 2311-2563. In some aspects, the modulator comprises a nucleic acid sequence comprising at least 80%, at least 85%, at least 90%, or at least 95% identical to a sequence selected from any one of SEQ ID Nos: 2311-2563. In some aspects, the modulator is a single stranded antisense oligonucleotide (ASO), and the ASO comprises at least 10, 11, 12, or 13 consecutive nucleotides with no more than 1, 2, or 3 mismatches from any one of SEQ ID Nos: 2311-2563. In some aspects, the modulator is a single stranded antisense oligonucleotide (ASO), and the ASO comprises at least 80%, at least 85%, at least 90%, at least 95% identical to any one of SEQ ID Nos: 2311-2563.

In some aspects, the modulator is an ASO. In some aspects, the oligonucleotide is an ASO. In some aspects, the ASO is a modified antisense oligonucleotide (modified ASO). In some aspects, the ASO or the modified ASO comprises a nucleic acid sequence at least 9, at least 10, at least 11, at least 12, at least 13, at least 14, or at least 15 consecutive nucleotides that are complementary to at least a portion of the lncRNAs disclosed herein. In some aspects, the modulator is a single stranded oligonucleotide (ASO), and the ASO or the modified ASO comprises a nucleic acid sequence at least 80%, at least 85%, at least 90%, at least 95% complementary to at least a portion of the lncRNAs disclosed herein.

In some aspects, the modulator is an ASO. In some aspects, the ASO is a modified or artificially synthesized ASO. In some aspects, the ASO or the modified or artificially synthesized ASO comprises at least 10 nucleotides, at least 11 nucleotides, at least 12 nucleotides, at least 13 nucleotides, at least 15 nucleotides, or at least 16 nucleotides. In some aspects, the ASO or the modified or artificially synthesized ASO comprises at least 13 nucleotides.

In some aspects, the ASO or the modified or artificially synthesized ASO comprises at most 22 nucleotides, at most 23 nucleotides, at most 24 nucleotides, at most 25 nucleotides, at most 26 nucleotides, at most 27 nucleotides, at most 28 nucleotides, at most 29 nucleotides, at most 30 nucleotides, at most 31 nucleotides, at most 32 nucleotides, at most 33 nucleotides, at most 34 nucleotides, at most 35 nucleotides, or at most 36 nucleotides. In some aspects, the ASO or the modified or artificially synthesized ASO comprises at most 35 nucleotides.

In some aspects, the ASO or the modified or artificially synthesized ASO is about 13-35 nucleotides, about 14-30 nucleotides, about 14-23 nucleotides, about 14-22 nucleotides, about 16-23 nucleotides, or about 16-22 nucleotides. In some aspects, the ASO or the modified or artificially synthesized ASO comprises 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, or 35 nucleotides. In some aspects, the ASO or the modified or artificially synthesized ASO comprises 21 nucleotides. In some aspects, the ASO or the modified ASO comprises 23 nucleotides.

In some aspects, the ASO is a modified antisense oligonucleotide (modified ASO). In some aspects, the modified ASO comprises one or more sugar modifications, one or more phosphate backbone modifications, one or more purine modifications, one or more pyrimidine modifications, or any combination thereof. In some aspects, the modified ASO comprises 2′-ribose sugar modification, a methoxyethyl (MOEs) modification, locked nucleic acid (LNA) modification, constrained ethyl (cEt) modification, or any combination thereof. In some aspects, the modified ASO comprises 2′-ribose sugar modification. In some aspects, the modification comprises an H, OR, R, halo, SH, SR, NH2, NHR, NR2, or CN, wherein R is an alkyl moiety. In some aspects, the modification at the 2′ hydroxyl group is a 2′-O-methyl modification or a 2′-O-methoxyethyl(2′-O-MOE) modification, 2′-halo modification, 2′-fluoro modification, 2′-O-aminopropyl modification. In some aspects, the modified ASO comprises methoxyethyl (MOEs) modification. In some aspects, the 2′-ribose sugar modification comprises 2′-O-Methoxyethyl (2′-MOE or MOE) modification. In some aspects, the modified ASO comprises locked nucleic acid (LNA) modification, ethylene nucleic acids (ENA), tricyclo-DNA, 2′ cyclic ethyl (CET), unlocked nucleic acid (UNA), and conformationally restricted nucleoside (CRN), or any combination thereof. In some instances, the at least one nucleic acid analogue comprises an LNA or a 2′-MOE-modified nucleotide. In some aspects, the modified ASO constrained ethyl (cEt) modification.

In some aspects, the modified ASO is a modified antisense GapmeR oligonucleotide. In some instances, the modified ASO comprises one or more LNAs. In some instances, the modified ASO comprises one or more 2′-O-MOE modified nucleotide, or combination thereof. In some aspects, the modified antisense GapmeR oligonucleotide comprises a central region of consecutive DNA nucleotides flanked by a 5′-wing region and 3′-wing region, wherein at least one of 5′-wing region and 3′-wing region comprises a nucleic acid analogue, wherein the nucleic acid analogue comprises one or more ribose modifications, one or more backbone modifications, one or more nucleobase modifications, or a combination thereof. In some aspects, the nucleic acid analogue comprises a 2′-O-Methoxyethyl(2′-MOE) modification. In some aspects, the nucleic acid analogue comprises a 2′-O-Methoxyethyl(2′-MOE) modification.

In some aspects, the nucleic acid analogue comprises a locked nucleic acid (LNA). In some aspects, the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA. In some aspects, the 5′-wing region comprises at least one LNAs. In some aspects, the 5′-wing region comprises at least two LNAs. In some aspects, the 5′-wing region comprises at least two LNAs. In some aspects, the 5′-wing region comprises three consecutive LNAs. In some aspects, the 3′-wing region comprises at least one LNA. In some aspects, the 3′-wing region comprises two consecutive LNAs.

In some instances, the 5′-wing region comprises at least two LNAs or 2′-methoxyethyl (MOE)-modified nucleotides. In some instances, the 5′-wing region comprises three, four, or five consecutive LNAs or 2′-methoxyethyl (MOE)-modified nucleotides. In some instances, the 3′-wing region comprises at least one LNA or a 2′-methoxyethyl (MOE)-modified nucleotide. In some instances, the 3′-wing region comprises two, three, four, or five consecutive LNAs or 2′-methoxyethyl (MOE)-modified nucleotides.

In some aspects, the ASO or the modified ASO comprises one or more phosphorothioate internucleotide linkages. In some aspects, the ASO or the modified ASO comprises one or more phosphorodithioate internucleotide linkages. In some aspects, the one or more phosphorothioate internucleotide linkages is a phosphorothioate backbone. In some aspects, the ASO or the modified ASO comprises one or more phosphate backbone modifications. In some aspects, the one or more phosphate backbone modifications comprise phosphorothioate linkage, methylphosphonate linkage, guanidinopropyl phosphoramidate linkage, or any combination thereof.

In some instances, at least one internucleotide linkage of the ASO is a phosphorothioate internucleotide linkage. In some instances, each internucleotide linkage of the ASO is a phosphorothioate internucleotide linkage.

In some aspects, the one or more purine modifications comprise 2,6-diaminopurin, 3-deaza-adenine, 7-deaza-guanine, 8-zaido-adenine, or any combination thereof. In some aspects, the one or more pyrimidine modifications comprise 2-thio-thymidine, 5-carboxamide-uracil, 5-methyl-cytosine, 5-ethynyl-uracil, or any combination thereof.

In some aspects, the ASO comprises a phosphorodiamidate morpholino oligomer (PMO). In some aspects, the modified ASO comprises a phosphorodiamidate morpholino oligomer (PMO).

In some aspects, the modified ASO comprises a GapmeR comprising a central region of consecutive DNA nucleotides flanked by a 5′-wing region and 3′-wing region, wherein at least one of 5′-wing region and 3′-wing region comprises a nucleic acid analogue. In some instances, 5′-wing region comprises 2 or 3 nucleotides, RNA mimics, nucleic acid analogues, or combination thereof. In some aspects, 3′-wing region comprises 2 or 3 nucleotides, RNA mimics, nucleic acid analogues, or combination thereof. In some aspects, 5′-wing region comprises 3 nucleotides or nucleic acid analogues, or combination thereof, and 3′-wing region comprises 2 nucleotides or nucleic acid analogues, or combination thereof. In some aspects, 5′-wing region comprises 2 nucleotides or nucleic acid analogues, or combination thereof, and 3′-wing region comprises 3 nucleotides or nucleic acid analogues, or combination thereof. In some aspects, 5′-wing region comprises 3 nucleotides or nucleic acid analogues, or combination thereof, and 3′-wing region comprises 3 nucleotides or nucleic acid analogues, or combination thereof. In some instances, 5′-wing region comprises 3 nucleic acid analogues, and 3′-wing region comprises 2 nucleic acid analogues.

In some aspects, the nucleic acid analogue comprises an LNA. In some aspects, the 5′-wing region of the GapmeR disclosed herein comprises at least one, two, three, four, or five LNA. In some aspects, the 5′-wing region of the GapmeR disclosed herein comprises at least two LNAs. In some aspects, the 5′-wing region of the GapmeR disclosed herein comprises two consecutive LNAs. In some aspects, the 5′-wing region of the GapmeR disclosed herein comprises at least three LNAs. In some aspects, the 5′-wing region of the GapmeR disclosed herein comprises three consecutive LNAs. In some aspects, the 5′-wing region of the GapmeR disclosed herein comprises at least four LNAs. In some aspects, the 5′-wing region of the GapmeR disclosed herein comprises four consecutive LNAs. In some aspects, the 5′-wing region of the GapmeR disclosed herein comprises five LNAs. In some aspects, the 5′-wing region of the GapmeR disclosed herein comprises five consecutive LNAs.

In some aspects, the 3′-wing region comprises an LNA. In some aspects, the 3′-wing region comprises at least two LNAs. In some aspects, the 3′-wing region comprises two consecutive LNAs. In some aspects, the 3′-wing region comprises at least three LNAs. In some aspects, the 3′-wing region comprises three consecutive LNAs.

In some aspects, the modified ASO is a modified antisense oligonucleotide comprising one or more nucleic acid analogue. In some instances, the one or more nucleic acid analogue comprises a 2′-O-Methoxyethyl(2′-MOE or MOE, used throughout interchangeably) modification. In some aspects, the modified ASO comprises a central region of consecutive DNA nucleotides flanked by a 5′-wing region and 3′-wing region, wherein at least one of 5′-wing region and 3′-wing region comprises a nucleic acid analogue, wherein the nucleic acid analogue comprises a 2′-MOE modification.

In some aspects, the 5′-wing region of the modified ASO comprises at least one 2′-MOE modified nucleotide. In some aspects, the 5′-wing region of the modified ASO comprises at least two 2′-MOE modified nucleotides. In some aspects, the 5′-wing region of the modified ASO comprises at least three 2′-MOE modified nucleotides. In some aspects, the 5′-wing region of the modified ASO comprises at least four 2′-MOE modified nucleotides. In some aspects, the 5′-wing region of the modified ASO comprises at least five 2′-MOE modified nucleotides. In some aspects, the 5′-wing region of the modified ASO comprises two consecutive 2′-MOE modified nucleotides. In some aspects, the 5′-wing region of the modified ASO comprises three consecutive 2′-MOE modified nucleotides. In some aspects, the 5′-wing region of the modified ASO comprises four consecutive 2′-MOE modified nucleotides. In some aspects, the 5′-wing region of the modified ASO comprises five consecutive 2′-MOE modified nucleotides. In some aspects, the 3′-wing region of the modified ASO comprises at least two 2′-MOE modified nucleotides. In some aspects, the 3′-wing region of the modified ASO comprises at least three 2′-MOE modified nucleotides. In some aspects, the 3′-wing region of the modified ASO comprises at least four 2′-MOE modified nucleotides. In some aspects, the 3′-wing region of the modified ASO comprises at least five 2′-MOE modified nucleotides. In some aspects, the 3′-wing region of the modified ASO comprises two consecutive 2′-MOE modified nucleotides. In some aspects, the 3′-wing region of the modified ASO comprises three consecutive 2′-MOE modified nucleotides. In some aspects, the 3′-wing region of the modified ASO comprises four consecutive 2′-MOE modified nucleotides. In some aspects, the 3′-wing region of the modified ASO comprises five consecutive 2′-MOE modified nucleotides.

In some aspects, one or more 2′-ribose sugar modifications are introduced to the ASOs described herein. In some embodiments, without binding to any specific theory, locked nucleic acid (LNA) increases the binding affinity with a 4-carbon tethered to a 2-hydroxyl group (2,4 bridged nucleic acid). In some embodiments, additional modification (e.g., constrained ethyl-cET) is built upon LNA chemistry and the both c-ET and LNA have similar target activity due to locking the 2′ and 4′ positions on the ribose ring. In some embodiments, 2′-O-Methoxyethyl(2′-MOE) increases binding activity by modifying the 2′ carbon.

In some aspects, one or more phosphodiester linkages in the ASOs described herein are modified with phosphorothioate linkage, resulting in phosphorothioate backbone connecting two nucleotides.

In some instances, the synthetic oligonucleotide is an ASO, wherein the ASO comprises i) a GapmeR comprising a central region of consecutive DNA nucleotides flanked by a 5′-wing region and 3′-wing region, wherein at least one of 5′-wing region and 3′-wing region comprises at least one nucleic acid analogue; and/or ii) a MixmeR of DNA and at least one nucleic acid analogue, or a combination thereof. In some instances, the synthetic oligonucleotide is an ASO, where the ASO comprises a GapmeR or a MixmeR of DNA. In some instances, the synthetic oligonucleotide is an ASO, where the ASO comprises a GapmeR and a MixmeR of DNA.

In some aspects, the modulator is an ASO. In some instances, the synthetic oligonucleotide is an ASO. In some aspects, the ASO is a modified ASO. In some instances, the modulator comprises a nucleic acid molecule comprising at least 10, at least 11, at least 12, or at least 13 consecutive nucleotides with no more than 1, no more than 2, or no more than 3 mismatches from any one of SEQ ID NOs: 1-2566 and 2580-3987, or comprises a nucleic acid sequence comprising at least 80%, at least 85%, at least 90%, or at least 95% identical to a sequence selected from any one of SEQ ID NOs: 1-2566 and 2580-3987. In some aspects, the ASO or the modified ASO comprises at least 10, at least 11, at least 12, or at least 13 consecutive nucleotides with no more than 1, no more than 2, or no more than 3 mismatches from any one of SEQ ID Nos: 1-2566 and 2580-3987. In some aspects, the ASO or the modified ASO comprises at least 10, at least 11, at least 12, or at least 13 consecutive nucleotides with no more than 1, no more than 2, or no more than 3 mismatches from any one of SEQ ID Nos: 1-28,31-37, 378, 385, 387, 2010-2018, and 2564-2566. In some instances, the modulator comprises a nucleic acid molecule comprising at least 10, at least 11, at least 12, or at least 13 consecutive nucleotides with no more than 1, no more than 2, or no more than 3 mismatches from any one of SEQ ID NOs: 1-28, 31-37, 378, 385, 387, 2010-2018, and 2564-2566, or comprises a nucleic acid sequence at least 80%, at least 85%, at least 90%, or at least 95% identical to a sequence selected from any one of SEQ ID NOs: 1-28, 31-37, 378, 385, 387, 2010-2018, and 2564-2566.

In some aspects, the ASO or the modified ASO comprises at least 8 consecutive nucleotides with no more than 8 mismatches, no more than 7 mismatches, no more than 6 mismatches, no more than 5 mismatches, no more than 4 mismatches, no more than 3 mismatches, no more than 2 mismatches or no more than 1 mismatch from any one of SEQ ID Nos: 1-2566 and 2580-3987. In some aspects, the ASO or the modified ASO comprises at least 9 consecutive nucleotides with no more than 7 mismatches, no more than 6 mismatches, no more than 5 mismatches, no more than 4 mismatches, no more than 3 mismatches, no more than 2 mismatches or no more than 1 mismatch from any one of SEQ ID Nos: 1-2566 and 2580-3987. In some aspects, the ASO or the modified ASO comprises at least 10 consecutive nucleotides with no more than 6 mismatches, no more than 5 mismatches, no more than 4 mismatches, no more than 3 mismatches, no more than 2 mismatches or no more than 1 mismatch from any one of SEQ ID Nos: 1-2566 and 2580-3987. In some aspects, the ASO or the modified ASO comprises at least 11 consecutive nucleotides with no more than 5 mismatches, no more than 4 mismatches, no more than 3 mismatches, no more than 2 mismatches or no more than 1 mismatch from any one of SEQ ID Nos: 1-2566 and 2580-3987. In some aspects, the ASO or the modified ASO comprises at least 12 consecutive nucleotides with no more than 4 mismatches, no more than 3 mismatches, no more than 2 mismatches or no more than 1 mismatch from any one of SEQ ID Nos: 1-2566 and 2580-3987. In some aspects, the ASO or the modified ASO comprises at least 13 consecutive nucleotides with no more than 3 mismatches, no more than 2 mismatches or no more than 1 mismatch from any one of SEQ ID Nos: 1-2566 and 2580-3987. In some aspects, the ASO or the modified ASO comprises at least 14 consecutive nucleotides with no more than 2 mismatches or no more than 1 mismatch from any one of SEQ ID Nos: 1-2566 and 2580-3987. In some aspects, the ASO or the modified ASO comprises 15 consecutive nucleotides with 1 mismatch from any one of SEQ ID Nos: 1-2566 and 2580-3987. In some aspects, the ASO or modified ASO comprises a nucleic acid sequence at least 80%, at least 85%, at least 90%, at least 95%, at least 96%, at least 97%, at least 98%, at least 99% identical to a sequence selected from any one of SEQ ID Nos: 1-2566 and 2580-3987. In some aspects, the ASO or the modified ASO disclosed herein comprises at least 8, at least 9, at least 10, at least 11, at least 12, at least 13, at least 14, at least 15 consecutive nucleotides selected from the group consisting of SEQ ID Nos: 1-2566 and 2580-3987.

In some instances, the modulator comprises a nucleic acid molecule comprising at least 10, at least 11, at least 12, or at least 13 consecutive nucleotides with no more than 1, no more than 2, or no more than 3 mismatches from any one of SEQ ID NOs: 1-2013, 2564-2566, and 2580-2947, or comprises a nucleic acid sequence at least 80%, at least 85%, at least 90%, or at least 95% identical to a sequence selected from any one of SEQ ID NOs: 1-2013, 2564-2566, and 2580-2947. In some instances, the modulator comprises a nucleic acid molecule comprising at least 10, at least 11, at least 12, or at least 13 consecutive nucleotides with no more than 1, no more than 2, or no more than 3 mismatches from any one of SEQ ID NOs: 2014-2563 and 3948-3987, or comprises a nucleic acid sequence at least 80%, at least 85%, at least 90%, or at least 95% identical to a sequence selected from any one of SEQ ID NOs: 2014-2563 and 3948-3987.

In some aspects, the ASO or the modified ASO comprises at least 8 consecutive nucleotides with no more than 8 mismatches, no more than 7 mismatches, no more than 6 mismatches, no more than 5 mismatches, no more than 4 mismatches, no more than 3 mismatches, no more than 2 mismatches or no more than 1 mismatch from any one of SEQ ID Nos: 1-28, 31-37, 378, 385, 387, 2010-2018, and 2564-2566. In some aspects, the ASO or the modified ASO comprises at least 9 consecutive nucleotides with no more than 7 mismatches, no more than 6 mismatches, no more than 5 mismatches, no more than 4 mismatches, no more than 3 mismatches, no more than 2 mismatches or no more than 1 mismatch from any one of SEQ ID Nos: 1-28,31-37, 378, 385, 387, 2010-2018, and 2564-2566. In some aspects, the ASO or the modified ASO comprises at least 10 consecutive nucleotides with no more than 6 mismatches, no more than 5 mismatches, no more than 4 mismatches, no more than 3 mismatches, no more than 2 mismatches or no more than 1 mismatch from any one of SEQ ID Nos: 1-28, 31-37, 378, 385, 387, 2010-2018, and 2564-2566. In some aspects, the ASO or the modified ASO comprises at least 11 consecutive nucleotides with no more than 5 mismatches, no more than 4 mismatches, no more than 3 mismatches, no more than 2 mismatches or no more than 1 mismatch from any one of SEQ ID Nos: 1-28, 31-37, 378, 385, 387, 2010-2018, and 2564-2566. In some aspects, the ASO or the modified ASO comprises at least 12 consecutive nucleotides with no more than 4 mismatches, no more than 3 mismatches, no more than 2 mismatches or no more than 1 mismatch from any one of SEQ ID Nos: 1-28, 31-37, 378, 385, 387, 2010-2018, and 2564-2566. In some aspects, the ASO or the modified ASO comprises at least 13 consecutive nucleotides with no more than 3 mismatches, no more than 2 mismatches or no more than 1 mismatch from any one of SEQ ID Nos: 1-28, 31-37, 378, 385, 387, 2010-2018, and 2564-2566. In some aspects, the ASO or the modified ASO comprises at least 14 consecutive nucleotides with no more than 2 mismatches or no more than 1 mismatch from any one of SEQ ID Nos: 1-28, 31-37, 378, 385, 387, 2010-2018, and 2564-2566. In some aspects, the ASO or the modified ASO comprises 15 consecutive nucleotides with 1 mismatch from any one of SEQ ID Nos: 1-28, 31-37, 378, 385, 387, 2010-2018, and 2564-2566. In some aspects, the ASO or modified ASO comprises a nucleic acid sequence at least 80%, at least 85%, at least 90%, at least 95%, at least 96%, at least 97%, at least 98%, at least 99% identical to a sequence selected from any one of SEQ ID Nos: 1-28,31-37, 378, 385, 387, 2010-2018, and 2564-2566. In some aspects, the ASO or the modified ASO disclosed herein comprises at least 8, at least 9, at least 10, at least 11, at least 12, at least 13, at least 14, at least 15 consecutive nucleotides selected from the group consisting of SEQ ID Nos: 1-28, 31-37, 378, 385, 387, 2010-2018, and 2564-2566.

In some aspects, the ASO or the modified ASO comprises at least 8 consecutive nucleotides with no more than 8, 7, 6, 5, 4, 3, 2, or 1 mismatches from any one of sequences set forth in Tables 1-16, 21-28, and 70-77. In some aspects, the ASO or the modified ASO comprises at least 9 consecutive nucleotides with no more than 7, 6, 5, 4, 3, 2, or 1 mismatches from any one of sequences set forth in Tables 1-16, 21-28, and 70-77. In some aspects, the ASO or the modified ASO comprises at least 10 consecutive nucleotides with no more than 6, 5, 4, 3, 2, or 1 mismatches from any one of sequences set forth in Tables 1-16, 21-28, and 70-77. In some aspects, the ASO or the modified ASO comprises at least 11 consecutive nucleotides with no more than 5, 4, 3, 2, or 1 mismatches from any one of sequences set forth in Tables 11-16, 21-28, and 70-77. In some aspects, the ASO or the modified ASO comprises at least 12 consecutive nucleotides with no more than 4, 3, 2, or 1 mismatches from any one of sequences set forth in Tables 1-16, 21-28, and 70-77. In some aspects, the ASO or the modified ASO comprises at least 13 consecutive nucleotides with no more than 3, 2, or 1 mismatches from any one of sequences set forth in Tables 1-16, 21-28, and 70-77. In some aspects, the ASO or the modified ASO comprises at least 14 consecutive nucleotides with no more than 2 mismatches or no more than 1 mismatch from any one of sequences set forth in Tables 1-16, 21-28, and 70-77. In some aspects, the ASO or the modified ASO comprises 15 consecutive nucleotides with 1 mismatch from any one of sequences set forth in Tables 1-16, 21-28, and 70-77. In some aspects, the ASO or modified ASO comprises a nucleic acid sequence at least 80%, at least 85%, at least 90%, at least 95%, at least 96%, at least 97%, at least 98%, at least 99% identical to a sequence selected from any one of sequences set forth in Tables 1-16, 21-28, and 70-77. In some aspects, the ASO or the modified ASO disclosed herein comprises at least 8, at least 9, at least 10, at least 11, at least 12, at least 13, at least 14, at least 15 consecutive nucleotides selected from the group consisting of sequences set forth in Tables 1-16, 21-28, and 70-77.

In some aspects, the ASO or the modified ASO comprises any one of sequences set forth in Tables 1-16, 21-28, and 70-77. In some instances, the ASO or the modified ASO comprises at least 9 consecutive nucleotides with no more than 1, 2, or 3 mismatches from sequences set forth in Tables 1-16, 21-28, and 70-77. In some instances, the ASO or the modified ASO comprises at least 10 consecutive nucleotides with no more than 1, 2, or 3 mismatches from sequences set forth in Tables 1-16, 21-28, and 70-77. In some instances, the ASO or the modified ASO comprises at least 11 consecutive nucleotides with no more than 1, 2, or 3 mismatches from sequences set forth in Tables 1-16, 21-28, and 70-77. In some instances, the ASO or the modified ASO comprises at least 12 consecutive nucleotides with no more than 1, 2, or 3 mismatches from sequences set forth in Tables 1-16, 21-28, and 70-77. In some instances, the ASO or the modified ASO comprises at least 13 consecutive nucleotides with no more than 1, 2, or 3 mismatches from sequences set forth in Tables 1-16, 21-28, and 70-77. In some instances, the ASO or the modified ASO comprises a nucleic acid sequence of 80%, at least 85%, at least 90%, at least 95% identical to sequences set forth in sequences set forth in Tables 1-16, 21-28, and 70-77.

Tables 1-16 show sequences of ASO candidates targeting lncRNAs disclosed herein and the criteria by which they were selected in silico. The ASO candidates were selected in silico based on the following criteria: 1) no off-target hybridization, 2) absence of questionable motifs, 3) target accessibility, 4) hybridization free energy, and 5) secondary structures.

### Gene Editing Modulator

In one aspect, the present disclosure provides a modulator that can edit or modify a target nucleic acid sequence. In some aspects, the target nucleic acid sequence comprises genomic DNA or RNA (e.g., lncRNA). The modulator comprises an endonuclease complex guided by a nucleic acid, wherein the nucleic acid targets the lncRNA or a genomic locus thereof.

In some instances, the modulator is a nucleic acid editing or modifying tool or a synthetic or artificial oligonucleotide. In some aspects, the modulator disclosed herein is a nucleic acid editing or modifying moiety. In some instances, the nucleic acid editing or modifying moiety targets the genomic region disclosed herein. In some instances, the nucleic acid editing or modifying moiety targets the lncRNA. In some instances, the nucleic acid editing or modifying moiety targets a premature form of the lncRNA.

In some aspects, the nucleic acid editing or modifying moiety is a programmable nucleic acid sequence specific endonuclease. In some instances, the nucleic acid editing or modifying moiety is a nucleic acid guided endonuclease. In some instances, the nucleic acid editing or modifying moiety is a CRISPR-based moiety. In other instances, the nucleic acid editing or modifying moiety is a meganuclease-based moiety. In other instances, the nucleic acid editing or modifying moiety is a zinc finger nuclease (ZFN)-based moiety. In other aspects, the nucleic acid editing or modifying moiety is a transcription activator-like effector-based nuclease (TALEN)-based moiety. In other instances, the nucleic acid editing or modifying tool is an Argonaute system.

In some aspects, the modulator comprises a CRISPR-based moiety. In some aspects, the CRISPR-based moiety is a CRISPR-directed DNA editing complex or a CRISPR-directed RNA editing complex.

In some aspects, the CRISPR-based moiety disclosed herein is a Type I, Type II, Type III, Type IV, Type V, or Type VI CRISPR system. CRISPR/Cas systems can be multi-protein systems or single effector protein systems. Multi-protein, or Class 1, CRISPR systems comprise Type I, Type III, and Type IV systems. In some instances, Class 2 systems comprise a single effector molecule and include Type II, Type V, and Type VI. In some aspects, the CRISPR-based tool disclosed herein comprises a single or multiple effector proteins. In some aspects, the effector protein can comprise one or multiple nuclease domains. In some aspects, the effector protein can target DNA or RNA, and the DNA or RNA can be single stranded or double stranded. In some aspects, the effector proteins can generate double strand or single strand breaks. In some aspects, the effector proteins comprise mutations in a nuclease domain thereby generating a nickase protein. In some aspects, the effector proteins comprise mutations in one or more nuclease domains, thereby generating a catalytically dead nuclease that is able to bind but not cleave a target sequence.

In some aspects, the CRISPR-based moiety disclosed comprises a single or multiple guiding RNAs (gRNAs). In some aspects, the gRNA disclosed herein targets a portion of chr21: 42,009,177-42,055,325 (strand +). In some aspects, the gRNA disclosed herein targets a portion of chr15: 38,864,806-39,427,195 (strand −). In some aspects, the gRNA disclosed herein targets a portion of chr15: 38,871,616-38,880,364 (strand −). In some aspects, the gRNA disclosed herein targets a portion of chr6: 169, 171,312-169,188,635 (strand −). In some aspects, the gRNA disclosed herein targets a portion of chr4: 76,758,554-76,801,964 (strand −). In some aspects, the gRNA disclosed herein targets a portion of chr17: 50,199,876-50,215,922 (strand +). In some aspects, the gRNA disclosed herein targets a portion of chr2: 215,717,811-215,721,161 (strand +). In some aspects, the gRNA disclosed herein targets a portion of chr14: 74,552,181-74,560,688 (strand +). In some aspects, the gRNA disclosed herein targets a portion of chr2: 188,966,458-188,984,794 (strand −). In some aspects, the gRNA disclosed herein targets a portion of chr11: 1,218,530-1,220,242 (strand +). In some aspects, the gRNA disclosed herein targets a portion of chr6: 74,069,451-74,690,727 (strand +). In some aspects, the gRNA disclosed herein targets a portion of chr16: 86,845,338-87,058,332 (strand −). In some aspects, the gRNA disclosed herein targets a portion of chr9: 22646200-22824213 (strand +).

In some aspects, the gRNA disclosed herein targets a portion of ENSG00000237232.9 (chr21: 42009177-42055325). In some aspects, the gRNA disclosed herein targets a portion of ENSG00000259345.7 (chr15: 38864806-39427195). In some aspects, the gRNA disclosed herein targets a portion of XLOC_029593 (chr15: 38871616-38880364). In some aspects, the gRNA disclosed herein targets a portion of ENSG00000261039.3 (chr6: 169171312-169188635). In some aspects, the gRNA disclosed herein targets a portion of ENSG00000224218.1 (chr4: 76758554-76801964). In some aspects, the gRNA disclosed herein targets a portion of ENSG00000249406.3 (chr17: 50199876-50215922). In some aspects, the gRNA disclosed herein targets a portion of ENSG00000230838.2 (chr2: 215717811-215721161). In some aspects, the gRNA disclosed herein targets a portion of ENSG00000258425.1 (chr14: 74552181-74560688). In some aspects, the gRNA disclosed herein targets a portion of XLOC_051798 (chr2: 188966458-188984794). In some aspects, the gRNA disclosed herein targets a portion of ENSG00000286275.1 (chr11: 1218530-1220242). In some aspects, the gRNA disclosed herein targets a portion of ENSG00000223786.1 (chr6: 74069451-74690727). In some aspects, the gRNA disclosed herein targets a portion of ENSG00000289423.1 (chr16: 86845338-87058332).

In some aspects, the gRNA comprises a crRNA. In some aspects, the gRNA comprises a chimeric RNA with crRNA and tracrRNA sequences. In some aspects, the gRNA comprises a separate crRNA and tracrRNA. In some aspects, target nucleic acid sequences comprise a protospacer adjacent motif (PAM) or a protospacer flanking site (PFS). In some aspects, the PAM or PFS can be 3′ or 5′ of the target or protospacer site. In some aspects, cleavage of a target sequence can generate blunt ends, 3′ overhangs, or 5′ overhangs.

In some aspects, the gRNA disclosed herein comprises a spacer sequence. In some aspects, the spacer sequences can be complementary to target sequences or protospacer sequences. In some aspects, the spacer sequences can be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some aspects, the spacer sequence can be less than 10 or more than 36 nucleotides in length.

In some aspects, the gRNA disclosed herein can comprise a repeat sequence. In some aspects, the repeat sequence is part of a double stranded portion of the gRNA. In some aspects, the repeat sequence can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some aspects, the spacer sequence can be less than 10 or more than 50 nucleotides in length.

In some aspects, the gRNA disclosed herein comprises one or more synthetic nucleotides, non-naturally occurring nucleotides, nucleotides with a modification, deoxyribonucleotide, or any combination thereof. In some aspects, the gRNA can comprise a hairpin, linker region, single stranded region, double stranded region, or any combination thereof. In some aspects, the gRNA can comprise a signaling or reporter molecule.

In some aspects, the gRNA disclosed herein can be encoded by genetic or episomal DNA. In some aspects, the gRNA disclosed herein can be provided or delivered concomitantly with a CRISPR nuclease or sequentially. In some aspects, the gRNA disclosed herein can be chemically synthesized, in vitro transcribed or otherwise generated using standard RNA generation techniques known in the art.

In some aspects, the CRISPR-based tool disclosed herein can be a Type II CRISPR system, for example a Cas9 system. In some aspects, the Type II nuclease can comprise a single effector protein, which comprises a RuvC and HNH nuclease domains. In some aspects, a functional Type II nuclease can comprise two or more polypeptides, each of which comprises a nuclease domain or fragment thereof. In some aspects, the target nucleic acid sequences can comprise a 3′ protospacer adjacent motif (PAM). In some aspects, the PAM can be 5′ of the target nucleic acid. In some aspects, guide RNAs (gRNA) can comprise a single chimeric gRNA, which contains both crRNA and tracrRNA sequences. In some instances, the gRNA can comprise a set of two RNAs, for example a crRNA and a tracrRNA. In some aspects, the Type II nuclease can generate a double strand break, which in some cases creates two blunt ends. In some aspects, the Type II CRISPR nuclease is engineered to be a nickase such that the nuclease only generates a single strand break. In such cases, in some aspects, the two distinct nucleic acid sequences can be targeted by gRNAs such that two single strand breaks are generated by the nickase. In some aspects, the two single strand breaks effectively create a double strand break. In some aspects where a Type II nickase is used to generate two single strand breaks, the resulting nucleic acid free ends can either be blunt, have a 3′ overhang, or a 5′ overhang. In some aspects, a Type II nuclease can be catalytically dead such that it binds to a target sequence, but does not cleave. For example, the Type II nuclease can have mutations in both the RuvC and HNH domains, thereby rendering both nuclease domains non-functional. In some aspects, the Type II CRISPR system can be one of three sub-types, namely Type II-A, Type II-B, or Type II-C.

In some aspects, the CRISPR-based tool disclosed herein can be a Type V CRISPR system, for example a Cpf1, C2c1, or C2c3 system. In some aspects, the Type V nuclease can comprise a single effector protein, which comprises a single RuvC nuclease domain. In some aspects, the function Type V nuclease comprises a RuvC domain split between two or more polypeptides. In some aspects, the target nucleic acid sequences can comprise a 5′ PAM or 3′ PAM. In some aspects, the guide RNAs (gRNA) can comprise a single gRNA or single crRNA, such as can be the case with Cpf1. In some aspects, a tracrRNA is not needed. In some aspects, such as when C2c1 is used, the gRNA can comprise a single chimeric gRNA, which contains both crRNA and tracrRNA sequences or the gRNA can comprise a set of two RNAs, for example a crRNA and a tracrRNA. In some aspects, the Type V CRISPR nuclease can generate a double strand break, which generates a 5′ overhang. In some aspects, the Type V CRISPR nuclease is engineered to be a nickase such that the nuclease only generates a single strand break. In some aspects, the two distinct nucleic acid sequences can be targeted by gRNAs such that two single strand breaks are generated by the nickase. In some aspects, the two single strand breaks effectively create a double strand break. In some aspects where a Type V nickase is used to generate two single strand breaks, the resulting nucleic acid free ends can either be blunt, have a 3′ overhang, or a 5′ overhang. In some aspects, a Type V nuclease can be catalytically dead such that it binds to a target sequence, but does not cleave. For example, the Type V nuclease can have mutations a RuvC domain, thereby rendering the nuclease domain non-functional.

In some aspects, the CRISPR-based tool disclosed herein can be a Type VI CRISPR system, for example a C2c2 system. In some aspects, the Type VI nuclease can comprise a HEPN domain. In some aspects, the Type VI nuclease comprises two or more polypeptides, each of which comprises a HEPN nuclease domain or fragment thereof. In some aspects, the target nucleic acid sequences can by RNA, such as single stranded RNA. When using Type VI CRISPR system, the target nucleic acid can comprise a protospacer flanking site (PFS). In some aspects, the PFS can be 3′ or 5′ or the target or protospacer sequence. In some aspects, the guide RNAs (gRNA) can comprise a single gRNA or single crRNA. In some aspects, a tracrRNA is not needed. In some aspects, the gRNA can comprise a single chimeric gRNA, which contains both crRNA and tracrRNA sequences or the gRNA can comprise a set of two RNAs, for example a crRNA and a tracrRNA. In some aspects, the Type VI nuclease can be catalytically dead such that it binds to a target sequence, but does not cleave. For example, the Type VI nuclease can have mutations in a HEPN domain, thereby rendering the nuclease domains non-functional.

Non-limiting examples of suitable nucleases, such as nucleic acid-guided nucleases, for use in the present disclosure include, but are not limited to, C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof.

In some aspects, the CRISPR-based tool disclosed herein is an Argonaute (Ago) system. In some aspects, the Ago system comprise an Ago protein. In some aspects, the Ago protein can be derived from a prokaryote, eukaryote, or archaea. In some aspects, the target nucleic acid can be RNA or DNA. A DNA target can be single stranded or double stranded. In some aspects, the target nucleic acid does not require a specific target flanking sequence, such as a sequence equivalent to a protospacer adjacent motif or protospacer flanking sequence. In some aspects, the Ago protein can create a double strand break or single strand break. In some aspects, when the Ago protein forms a single strand break, two Ago proteins can be used in combination to generate a double strand break. In some aspects, the Ago protein comprises one, two, or more nuclease domains. In some aspects, the Ago protein comprises one, two, or more catalytic domains. In some aspects, one or more nuclease or catalytic domains can be mutated in the Ago protein, thereby generating a nickase protein capable of generating single strand breaks. In other embodiments, mutations in one or more nuclease or catalytic domains of an Ago protein generates a catalytically dead Ago protein that can bind but not cleave a target nucleic acid.

In some aspects, the Ago proteins can be targeted to target nucleic acid sequences by a guiding nucleic acid. In some aspects, the guiding nucleic acid is a guide DNA (gDNA). In some aspects, the gDNA can have a 5′ phosphorylated end. In some aspects, the gDNA can be single stranded or double stranded. In some aspects, the single stranded gDNA can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some aspects, the gDNA can be less than 10 nucleotides in length. In some aspects, the gDNA can be more than 50 nucleotides in length.

In some aspects, Argonaute-mediated cleavage can generate blunt end, 5′ overhangs, or 3′ overhangs. In some aspects, one or more nucleotides are removed from the target site during or following cleavage.

In some aspects, the nucleic acid editing or modifying tool is a repressive dCas9 with the aid of a (single) guide RNA targeting the portion of the genomic region that is transcribed to the lncRNA. In some instances, the nucleic acid editing or modifying tool is dCas9-KRAB-MECP2 with the aid of a (single) guide RNA targeting the portion of the genomic region that is transcribed to the lncRNA. In other embodiments, the nucleic acid editing or modifying tool is dCas9-KRAB-DNMT1 with the aid of a (single) guide RNA targeting the portion of the genomic region that is transcribed to the lncRNA. In some aspects, the (single) guide RNA targets 5′ side of an enhancer region the genomic region that is transcribed to the lncRNA. In some aspects, the (single) guide RNA targets 5′ side of an enhancer region the genomic region that is transcribed to the lncRNA.

### Pharmaceutical Compositions and Kits

Further provided herein are pharmaceutical compositions comprising the modulator disclosed herein and a pharmaceutically acceptable salt, excipient, or derivative thereof.

In some aspects, the pharmaceutically acceptable salts, excipient, or derivative thereof comprise but are not limited to (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

In some aspects, the pharmaceutical composition described herein can be prepared to comprise the modulator disclosed herein, into a form suitable for administration to a subject using carriers, excipients, and vehicles. In some instances, excipients comprise magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. In some aspects, intravenous vehicles comprise fluid and nutrient replenishers. In some aspects, preservatives comprise antimicrobial, anti-oxidants, chelating agents, and inert gases. In some aspects, other pharmaceutically acceptable vehicles comprise aqueous solutions, non-toxic excipients, including salts, preservatives, buffers and the like, as described, for instance, in Remington: The Science and Practice of Pharmacy, 21st Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

In some aspects, the pharmaceutical compositions described herein can be administered locally or systemically. In some aspects, the therapeutically effective amounts will vary according to factors, such as the degree of infection in a subject, the age, sex, health conditions, and weight of the individual. In some aspects, dosage regimes can be adjusted to provide the optimum therapeutic response. For example, several divided doses can be administered daily, or the dose can be proportionally reduced as indicated by the exigencies of the therapeutic situation.

In some aspects, the pharmaceutical composition can be administered in a convenient manner, such as by injection (e.g., subcutaneous, intravenous, intraorbital, and the like), oral administration, ophthalmic application, inhalation, topical application, or rectal administration. Depending on the route of administration, in some aspects, the pharmaceutical composition can be coated with a material to protect the pharmaceutical composition from the action of enzymes, acids, and other natural conditions that can inactivate the pharmaceutical composition. In some aspects, the pharmaceutical composition can also be administered parenterally or intraperitoneally. In some aspects, dispersions can also be prepared in glycerol, liquid polyethylene glycols, and mixtures thereof, and in oils. Under ordinary conditions of storage and use, in some aspects, these preparations can contain a preservative to prevent the growth of microorganisms.

In some aspects, the pharmaceutical compositions suitable for injectable use comprise sterile aqueous solutions (where water soluble) or dispersions and sterile powders for the extemporaneous preparation of sterile injectable solutions or dispersions. In some aspects, the pharmaceutical composition can be sterile and fluid to the extent that easy syringeability exists. In some aspects, the pharmaceutical composition can be stable under the conditions of manufacture and storage and preserved against the contaminating action of microorganisms, such as bacteria and fungi. In some aspects, vehicle can be a solvent or dispersion medium comprising, for example, water, ethanol, polyol (for example, glycerol, propylene glycol, and liquid polyethylene glycol, and the like), suitable mixtures thereof, and vegetable oils. In some aspects, the proper fluidity can be maintained, for example, by the use of a coating, such as lecithin, by the maintenance of certain particle size, in the case of dispersion, and by the use of surfactants. In some aspects, prevention of the action of microorganisms can be achieved by various antibacterial and antifungal agents, for example, parabens, chlorobutanol, phenol, ascorbic acid, thimerosal, and the like. In some aspects, isotonic agents, for example, sugars, polyalcohols, such as mannitol, sorbitol, or sodium chloride are used in the composition. In some aspects, prolonged absorption of the injectable compositions can be brought about by including in the composition an agent that delays absorption, for example, aluminum monostearate and gelatin.

In some aspects, sterile injectable solutions can be prepared by incorporating the pharmaceutical composition in an appropriate solvent with one or a combination of ingredients enumerated above followed by filtered sterilization. In some aspects, dispersions are prepared by incorporating the pharmaceutical composition into a sterile vehicle that contains a basic dispersion medium and the other ingredients from those enumerated above.

In some aspects, the pharmaceutical composition can be formulated as parenteral compositions. In some aspects, the parenteral compositions in dosage unit form can provide ease of administration and uniformity of dosage. In some aspects, the dosage unit form as used herein, refers to physically discrete units suited as unitary dosages for the subject to be treated; each unit comprising a predetermined quantity of pharmaceutical composition is calculated to produce the desired therapeutic effect in association with the pharmaceutical vehicle. In some aspects, the specification for the dosage unit forms is related to the characteristics of the pharmaceutical composition and the particular therapeutic effect to be achieve. In some aspects, the pharmaceutical composition is compounded for convenient and effective administration in effective amounts with a suitable pharmaceutically acceptable vehicle in an acceptable dosage unit. In some aspects, in the case of compositions comprising supplementary active ingredients, the dosages are determined by reference to the usual dose and manner of administration of the ingredients.

In some aspects, the pharmaceutical composition can be orally administered, for example, in a carrier, e.g., in an enteric-coated unit dosage form. In some aspects, the pharmaceutical composition and other ingredients can also be enclosed in a hard or soft-shell gelatin capsule or compressed into tablets. For oral therapeutic administration, in some aspects, the pharmaceutical composition can be incorporated with excipients and used in the form of ingestible tablets, troches, capsules, pills, wafers, and the like. In some aspects, the pharmaceutical compositions and preparations can contain at least 1% by weight of active compound. In some aspects, the percentage of the pharmaceutical compositions and preparations can, of course, be varied and can conveniently be between about 5% to about 80% of the weight of the unit. In some aspects, the tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, in some aspects, it can comprise, in addition to materials of the above type, a liquid carrier. In some aspects, various other materials can be present as coatings or to otherwise modify the physical form of the dosage unit. For instance, tablets, pills, or capsules can be coated with shellac, sugar, or both. In some aspects, a syrup or elixir can contain the agent, sucrose as a sweetening agent, methyl and propylparabens as preservatives, a dye, and flavoring, such as cherry or orange flavor. Any material used in preparing any dosage unit form, in some aspects, can be of pharmaceutically acceptable purity and substantially non-toxic in the amounts employed. In some aspects, the pharmaceutical composition can be incorporated into sustained-release preparations and formulations.

In some aspects, the pharmaceutical composition described herein comprises one or more permeation enhancer that facilitates bioavailability of the modulator described herein. WO 2000/67798, Muranishi, 1990, Crit. Rev. Ther. Drug Carrier Systems, 7, 1, Lee et al., 1991, Crit. Rev. Ther. Drug Carrier Systems, 8, 91 are herein incorporated by reference in its entirety. In some aspects, the permeation enhancer is intestinal. In some aspects, the permeation enhancer is transdermal. In some aspects, the permeation enhancer is to facilitate crossing the brain-blood barrier. In some aspects, the permeation enhancer improves the permeability in the oral, nasal, buccal, pulmonary, vaginal, or corneal delivery model. In some aspects, the permeation enhancer is a fatty acid or a derivative thereof. In some aspects, the permeation enhancer is a surfactant or a derivative thereof. In some aspects, the permeation enhancer is a bile salt or a derivative thereof. In some aspects, the permeation enhancer is a chelating agent or a derivative thereof. In some aspects, the permeation enhancer is a non-chelating non-surfactant or a derivative thereof. In some aspects, the permeation enhancer is an ester or a derivative thereof. In some aspects, the permeation enhancer is an ether or a derivative thereof. In some specific aspects, the permeation enhancer is arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof. In one aspect, the permeation enhancer is sodium caprate (C10). In some instances, the permeation enhancer is chenodeoxycholic acid (CDCA), ursodeoxychenodeoxycholic acid (UDCA), cholic acid, dehydrocholic acid, deoxycholic acid, glucholic acid, glycholic acid, glycodeoxycholic acid, taurocholic acid taurodeoxycholic acid, sodium tauro-24,25-dihydro-fusidate or sodium glycodihydrofusidate. In some instances, the permeation enhancer is polyoxyethylene-9-lauryl ether, or polyoxyethylene-20-cetyl ether.

In some aspects, the pharmaceutical composition described herein comprise the modulator. In some aspects, the modulator is encapsulated in liposome or coupled with a nanoparticle. In some aspects, the modulator is encapsulated in liposome. In some aspects, the modulator is coupled with a nanoparticle.

In another aspect, the present disclosure provides a pharmaceutical composition comprising an expression vector. In some aspects, the expression vector comprises a transgene. In some aspects, the transgene encodes a modulator described herein. In some aspects, the modulator targets one or more lncRNAs. In some aspects, the one or more lncRNAs are transcribed from a region located within a genomic locus selected from any one of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +). In some aspects, the one or more lncRNAs are transcribed from a region located within a genomic locus selected from any one of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); and chr16: 86845338-87058332 (strand −).

Further provided herein are kits comprising the modulator disclosed herein. Further provided herein are kits comprising the pharmaceutical composition disclosed herein. In some aspects, the kit comprises suitable instructions in order to perform the methods of the kit. The instructions can provide information of performing any of the methods disclosed herein, whether or not the methods can be performed using only the reagents provided in the kit.

For use in the therapeutic applications described herein, kits and articles of manufacture are also described herein. In some aspects, such kits comprise a carrier, package, or container that is compartmentalized to receive one or more containers such as vials, tubes, and the like, each of the container(s) including one of the separate elements to be used in a method described herein. Suitable containers comprise, for example, bottles, vials, syringes, and test tubes. The containers can be formed from a variety of materials such as glass or plastic. The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. In some aspects, the container(s) have a sterile access port (for example the container is an intravenous solution bag or a vial having a stopper pierceable by a hypodermic injection needle). Such kits, in some aspects, comprise a composition with an identifying description or label or instructions relating to its use in the methods described herein.

A kit can comprise one or more additional containers, each with one or more of various materials (such as reagents, optionally in concentrated form, and/or devices) desirable from a commercial and user standpoint for use of the modulator described herein. Non-limiting examples of such materials include, but are not limited to, buffers, diluents, filters, needles, syringes, carrier, package, container, vial and/or tube labels listing contents and/or instructions for use, and package inserts with instructions for use. In some instances, a set of instructions is comprised.

In some aspects, a label is on or associated with the container. In some aspects, the label can be on a container when letters, numbers or other characters forming the label are attached, molded or etched into the container itself; a label can be associated with a container when it is present within a receptacle or carrier that also holds the container, e.g., as a package insert. In some aspects, the label can be used to indicate that the contents are to be used for a specific therapeutic application. The label can also indicate directions for use of the contents, such as in the methods described herein.

In some aspects, the pharmaceutical composition comprising the modulators provided herein and additional active agent is presented in a pack or dispenser device which can contain one or more unit dosage forms. The pack can, for example, contain metal or plastic foil, such as a blister pack. In some aspects, the pack or dispenser device can be accompanied by instructions for administration. In some aspects, the pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. In some aspects, the pharmaceutical compositions comprising the modulators described herein formulated in a compatible pharmaceutical carrier can also be prepared, placed in an appropriate container, and labeled for treatment of an indicated condition.

### Methods and Uses

In one aspect, the present disclosure provides methods of modulating the expression or activity of lncRNA disclosed herein by contacting a cell comprising and/or expressing the lncRNA to the modulator disclosed herein, or a pharmaceutical composition comprising the modulator disclosed herein. In some instances, the cell is affected by IPF. Also provided herein comprises methods of modulating the expression or activity of lncRNA disclosed herein in a subject by administering to the subject the modulator disclosed herein, or a pharmaceutical composition disclosed herein. In some instances, the subject has been developing IPF, affected by IPF or suffering from one or more symptoms of IPF. In some instances, the subject is a cell, a tissue, an animal, or a human.

In some aspects, the present disclosure provides methods of modulating one or more lncRNAs, the method comprising administering an effective amount of the modulator disclosed herein or a pharmaceutical composition disclosed herein to a subject in need thereof. In certain instances, the modulation of one or more lncRNAs is decreasing the expression or suppressing the expression or activity of the one or more lncRNAs. In some instances, the modulator modulates expression or activity of one or more pulmonary-specific lncRNAs in the subject. In some aspects, the one or more lncRNAs are pulmonary-specific lncRNAs. In some aspects, the pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +). In some aspects, the pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); and chr16: 86845338-87058332 (strand −).

In some aspects, the present disclosure provides a method of modulating expression or activity of one or more pulmonary-specific lncRNAs in a subject, comprising administering an effective amount of the modulator described herein or the pharmaceutical composition described herein to the subject in need thereof, wherein the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); and chr16: 86845338-87058332 (strand −). In some instances, the modulator further modulates RNA expression level, protein expression level, or both, of one or more genes from Table 18. In some instances, the modulator reduces RNA expression level, protein expression level, or both, of the one or more genes from Table 18. In some instances, the modulator reduces RNA expression level, protein expression level, or both, of one or more fibrotic genes selected from a group consisting of COL3A1, COL1A1, ACTA2, POSTN, FN1, FAP, and CTHRC1.

In some instances, the modulator further modulates one or more genes selected from a group consisting of i) MX1, MX2, thrombospondin 1 (THBS1), thrombospondin 2 (THBS2), or a combination thereof, ii) B9D1, IQCG, LRRC23, TMEM231, SPACA9, CCDC65, ARHGAP39, C9orf116, CFAP300, RAB36, or a combination thereof, iii) CCDC60, FAM81B, TCTE1, MDH1B, RSPH9, PACRG, C9orf116, ODAD4, TMEM231, or a combination thereof, and iv) CERCAM, COL3A1, COL1A1, COL5A2, COL1A2, CIS, ROR2, EPHB2, COL15A1, CTHRC1, or a combination thereof.

In some instances, the modulator restores a cytokine level to a level similar to a reference cytokine level obtained from a healthy subject. In some instances, the cytokine comprises IL-9, IL-1a, MIP-la (CCL3), G-CSF, IL-2, IL-7, or a combination thereof.

In some instances, the modulator inhibits epithelial to mesenchymal transition (EMT) of pulmonary cells. In some instances, the pulmonary cells comprise pulmonary fibroblasts, pulmonary myofibroblasts, or epithelial cells.

In another aspect, provided herein are methods of reducing expression or activity of the lncRNA disclosed herein in a subject in need thereof by administering to the subject the modulator disclosed herein, or a pharmaceutical composition disclosed herein. In some instances, the subject has been developing IPF, affected by IPF, or suffering from one or more symptoms of IPF.

In some aspect, provided herein are methods of preventing, alleviating, or treating pulmonary fibrosis or a symptom associated with pulmonary fibrosis in a subject in need thereof by administering to the subject an effective amount of the modulator disclosed herein or the pharmaceutical composition provided herein. In some instances, the pulmonary fibrosis is IPF. In some instances, the symptom comprises inflammation. In some instances, the inflammation is associated with onset or development of pulmonary fibrosis. In some instances, the inflammation is associated with onset or development of IPF.

Further provided herein are methods of preventing, alleviating, or treating pulmonary fibrosis or a symptom associated with such fibrosis in a subject in need thereof, the method comprising administering to the subject an effective amount of the modulator disclosed herein of a lncRNA transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +), or the pharmaceutical composition that modulates a lncRNA transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +). In some instances, the lncRNA comprising sequence transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +) is expressed at a moderate level in some tissues of mesenchymal origin such as lung.

In some aspects, the effective amount of a modulator as disclosed herein can decrease the amount, expression level (e.g., RNA expression level), or activity of the lncRNA disclosed herein at least 1%, 2%, 3%, 4%, 5%, 10%, 20%, 30%, 40%, 50%, 60%, 70%, 80%, 90% or more in the cell contacted with the modulator. In some aspects, the effective amount of a modulator as disclosed herein can decrease the amount, expression level (e.g., RNA expression level), or activity of the lncRNA disclosed herein at least 1%, 2%, 3%, 4%, 5%, 10%, 20%, 30%, 40%, 50%, 60%, 70%, 80%, 90% or more in the cells or tissues of the subject affected by pulmonary fibrosis, e.g., IPF.

In some aspects, the effective amount of a modulator as disclosed herein can modulate the amount, expression level (e.g., RNA expression level), or activity of transcripts or mRNAs of genes associated with or markers of pulmonary fibrosis, e.g., IPF in at least 1%, 2%, 3%, 4%, 5%, 10%, 20%, 30%, 40%, 50%, 60%, 70%, 80%, 90% or more in the cells or tissues of the subject affected by pulmonary fibrosis, e.g., IPF. In some aspects, the effective amount of the modulator disclosed herein can increase the amount, expression level, or activity of transcripts or mRNAs of at least one or more genes associated with or markers of pulmonary fibrosis, e.g., IPF in at least 1%, 2%, 3%, 4%, 5%, 10%, 20%, 30%, 40%, 50%, 60%, 70%, 80%, 90% or more in the cells or tissues of the subject affected by pulmonary fibrosis, e.g., IPF. In some aspects, the effective amount of a modulator as disclosed herein can decrease the amount, expression level, or activity of transcripts or mRNAs of at least one or more genes associated with or markers of pulmonary fibrosis, e.g., IPF in at least 1%, 2%, 3%, 4%, 5%, 10%, 20%, 30%, 40%, 50%, 60%, 70%, 80%, 90% or more in the cells or tissues of the subject affected by pulmonary fibrosis, e.g., IPF.

In some aspects, the effective amount of a modulator as disclosed herein can alleviate or reduce the severity or frequencies of symptoms of pulmonary fibrosis, e.g., IPF, or severity or frequencies of symptoms of IPF. In some aspects, the effective amount of a modulator as disclosed herein can slow down severity or frequencies symptoms of pulmonary fibrosis, e.g., IPF, or severity or symptoms of IPF, or progress of pulmonary fibrosis, e.g., IPF. In some aspects, the effective amount of a modulator as disclosed herein can reverse severity or frequencies symptoms of pulmonary fibrosis, e.g., IPF, or severity or symptoms of IPF, or progress of pulmonary fibrosis, e.g., IPF.

For delivery to the target cell, in some aspects, the modulator described herein can non-covalently bind an excipient to form a complex. In some aspects, the excipient can be used to alter biodistribution after delivery, to enhance uptake, to increase half-life or stability of the strands in the modulator described herein (e.g., improve nuclease resistance), and/or to increase targeting to a particular cell or tissue type. Non-limiting examples of excipients include, but are not limited to, a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include but are not limited to polyamines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly (L-lactide-co-glycolic) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; and a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

In some aspects, the administering is performed intratracheally, orally, nasally, intravenously, intraperitoneally, or intramuscularly. In some aspects, the administering is performed intratracheally.

In some aspects, the administering is a targeted delivery to a lung tissue of the subject. In some instances, the targeted delivery is via a local application. In some instances, the targeted delivery is via one or more specific binding moieties that target the lung tissue.

In some aspects, the administering is in a form of aerosol. In some instances, the aerodynamic diameter of particles of the modulator disclosed herein is less than 10 μm. In some instances, the aerodynamic diameter of particles of the modulator disclosed herein is less than 5 μm. In some instances, the aerodynamic diameter of particles of the modulator disclosed herein is less than 3 μm.

In another aspect, the present disclosure provides a method of i) diagnosing idiopathic pulmonary fibrosis (IPF), or ii) monitoring, determining or predicting severity or progression of idiopathic pulmonary fibrosis (IPF) in a subject. In some instances, the method comprises obtaining a sample from the subject, and determining an RNA expression level of (i) a plurality of genes selected from a group consisting of COL3A1, COL1A1, ACTA2, POSTN, FN1, FAP, CTHRC1, THBS1, THBS2, or (ii) one or more pulmonary-specific lncRNAs. Based on the RNA expression level measured in such step, idiopathic pulmonary fibrosis (IPF) can be diagnosed in the subject, or severity or progression of idiopathic pulmonary fibrosis (IPF) can be monitored, determined or predicted in the subject.

In some instances, RNA expression level is determined by comparing the RNA expression level of the set of genes from to a first reference RNA expression level. In some instances, the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +). In some instances, the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr17: 50199876-50215922 (strand +), and chr2: 215717811-215721161 (strand +).

In one aspect, provided herein are methods of diagnosing or monitoring IPF in a subject. In some instances, the method comprises obtaining a sample from the subject, and measuring an amount and/or an activity of a lncRNA, wherein the lncRNA is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +). In some instances, the subject is diagnosed with pulmonary fibrosis or evaluated/determined to have a high/higher chance to develop pulmonary fibrosis if the amount and/or the activity is at least 50%, at least 60%, at least 70%, at least 80%, at least 90% higher when compared to an amount and/or an activity of a lncRNA obtained from a control. In some instances, the control is a healthy subject or a non-disease subject. In some instances, the control is a group of healthy subjects or non-disease subjects. In some instances, the control is a subject before the treatment, a healthy subject, or a reference sample not affected by idiopathic pulmonary fibrosis. In some instances, the subject is a cell, a tissue, an animal, or a human. In some aspects, the measuring comprises using S1 nuclease protection assay, microarray analysis, polymerase chain reaction (PCR), hybridization technologies, reverse transcriptase polymerase chain reaction (RT-PCR), RNA-Seq, Northern blot, serial analysis of gene expression (SAGE), immunoassay, and/or mass spectrometry.

In another aspect, the present disclosure provides a method of treating idiopathic pulmonary fibrosis (IPF) in a subject by administering the subject an effective amount of the modulator described herein, or the pharmaceutical composition described herein, thereby treating the idiopathic pulmonary fibrosis in the subject. In some instances, the subject to be treated can be selected by obtaining a sample of the subject, measuring from the sample (a) RNA expression level, protein expression level, or both, of a plurality of genes from a set of genes setting forth in Table 18; or (b) RNA expression level of one or more pulmonary-specific lncRNAs, and selecting the subject for the treatment based on the measurement of (a) or (b).

In some instances, the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +).

In one aspect, the present disclosure provides a method of treating idiopathic pulmonary fibrosis (IPF) in a subject, the method comprises: (a) obtaining a sample from the subject; (b) measuring an amount and/or an activity of a lncRNA, wherein the lncRNA is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +); (c) diagnosing the subject with pulmonary fibrosis if the amount and/or the activity is at least 50%, at least 60%, at least 70%, at least 80%, at least 90% higher when compared to an amount and/or an activity of a lncRNA obtained from a control; and (d) treating the subject with an effective amount of the modulators disclosed herein.

In another aspect, the present disclosure provides a method of treating idiopathic pulmonary fibrosis (IPF) in a subject, the method comprising: (a) obtaining a sample from the subject; (b) measuring RNA expression level, protein expression level, or both, of a set of genes setting forth in Table 18; (c) measuring RNA expression level of one or more pulmonary-specific lncRNAs, wherein the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +); (d) diagnosing the subject with IPF; and (e) treating the subject with the modulator or the pharmaceutical composition described herein.

In one aspect, the present disclosure provides a method of predicting severity and progression of idiopathic pulmonary fibrosis (IPF) in a subject, the method comprising: (a) obtaining a sample from the subject; (b) measuring RNA expression of a set of genes comprising COL3A1, COL1A1, ACTA2, POSTN, FN1, FAP, CTHRC1, or a combination thereof; (c) measuring RNA expression of one or more pulmonary-specific lncRNAs, wherein the one or more pulmonary-specific lncRNAs transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +); thereby predicting severity and progression of IPF in the subject.

In various aspects, the present disclosure provides a method of determining an efficacy of a treatment for idiopathic pulmonary fibrosis, comprising: comparing a first RNA expression level of a subject after the treatment and a second RNA expression level of a control, wherein the first and second RNA expression levels comprise an RNA expression level of (i) a plurality of genes selected from a group consisting of COL3A1, COL1A1, ACTA2, POSTN, FN1, FAP, CTHRC1, THBS1, THBS2, or (ii) one or more pulmonary-specific lncRNAs, In some instances, the control is a subject before the treatment, a healthy subject, or a reference sample not affected by idiopathic pulmonary fibrosis. In some instances, the pulmonary-specific lncRNAs are transcribed from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +).

In another aspect, the present disclosure provides a method of rating severity and/or evaluating and/or predicting progression of idiopathic pulmonary fibrosis (IPF) in a subject, the method comprising: (a) obtaining a sample from the subject; (b) measuring an RNA expression level of a set of genes comprising COL3A1, COL1A1, ACTA2, POSTN, FN1, FAP, CTHRC1, THBS1, THBS2, or combination thereof; (c) measuring an RNA expression level of one or more pulmonary-specific lncRNAs, wherein the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +); and (d) computing i) an increased RNA expression level of the set of genes from (b) in comparison to a first reference RNA expression level and ii) an increased RNA expression level of the one or more pulmonary-specific lncRNAs from (c) in comparison to a second reference RNA expression level, thereby rating severity and/or evaluating and/or predicting progression of IPF in the subject. In some instances, the one or more pulmonary-specific lncRNAs are transcribed from a region located within a genomic locus selected from a group consisting of chr21: 42009177-42055325 (strand +), chr15: 38864806-39427195 (strand −), chr15: 38871616-38880364 (strand −), chr6: 169171312-169188635 (strand −), chr17: 50199876-50215922 (strand +), and chr2: 215717811-215721161 (strand +). In some instances, the one or more pulmonary-specific lncRNAs are transcribed from a region located within a genomic locus selected from any one of chr15: 38864806-39427195 (strand −) and chr15: 38871616-38880364 (strand −).

In some instances, the increased RNA expression level of the one or more pulmonary-specific lncRNAs from (c) is at least about 50%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, or at least about 1000% higher in comparison to the second reference RNA expression level. In some instances, the increased RNA expression level of the one or more pulmonary-specific lncRNAs from (c) is at most about 50%, at most about 100%, at most about 150%, at most about 200%, at most about 250%, at most about 300%, at most about 350%, at most about 400%, at most about 450%, at most about 500%, at most about 550%, at most about 600%, at most about 650%, at most about 700%, at most about 750%, at most about 800%, at most about 850%, at most about 900%, at most about 950%, or at most about 1000% higher in comparison to the second reference RNA expression level. In some instances, the increased RNA expression level of the one or more pulmonary-specific lncRNAs from (c) is at about 50%, at about 100%, at about 150%, at about 200%, at about 250%, at about 300%, at about 350%, at about 400%, at about 450%, at about 500%, at about 550%, at about 600%, at about 650%, at about 700%, at about 750%, at about 800%, at about 850%, at about 900%, at about 950%, or at about 1000% higher in comparison to the second reference RNA expression level. In some instances, the increased RNA expression level of the one or more pulmonary-specific lncRNAs from (c) is from about 50% to about 1100%, from about 100% to about 1100%, from about 150% to about 1100%, from about 200% to about 1100%, from about 250% to about 1100%, from about 300% to about 1100%, from about 350% to about 1100%, from about 400% to about 1100%, from about 450% to about 1100%, from about 500% to about 1100%, from about 550% to about 1100%, from about 600% to about 1100%, from about 650% to about 1100%, from about 700% to about 1100%, from about 750% to about 1100%, from about 800% to about 1100%, from about 850% to about 1100%, from about 900% to about 1100%, from about 950% to about 1100%, or from about 1000% to about 1100% higher in comparison to the second reference RNA expression level.

In some instances, the increased RNA expression level of the one or more pulmonary-specific lncRNAs from (c) is at least about 0.1-fold, at least about 0.5 fold, at least about 1 fold, at least about 2 folds, or at least about 5 folds, at least about 10 folds, at least about 50 folds, at least about 100 folds, or at least about 200 folds higher in comparison to the second reference RNA expression level. In some instances, the increased RNA expression level of the one or more pulmonary-specific lncRNAs from (c) is at most about 0.5 fold, at most about 1 fold, at most about 2 folds, or at most about 5 folds, at most about 10 folds, at most about 50 folds, at most about 100 folds, or at most about 200 folds higher in comparison to the second reference RNA expression level. In some instances, the increased RNA expression level of the one or more pulmonary-specific lncRNAs from (c) is at about 0.1-fold, at about 0.5 fold, at about 1 fold, at about 2 folds, or at about 5 folds, at about 10 folds, at about 50 folds, at about 100 folds, or at about 200 folds higher in comparison to the second reference RNA expression level. In some instances, the increased RNA expression level of the one or more pulmonary-specific lncRNAs from (c) is from about 0.1 fold to about 250 folds, from about 0.5 fold to about 250 folds, from about 1 fold to about 250 folds, from about 2 folds to about 250 folds, or from about 5 folds to about 250 folds, from about 10 folds to about 250 folds, from about 50 folds to about 250 folds, from about 100 folds to about 250 folds, or from about 200 folds to about 250 folds higher in comparison to the second reference RNA expression level.

In some instances, the first reference RNA expression level and the second reference RNA expression level are obtained from a control. In some instances, the control is a healthy subject or a non-disease subject. In some instances, the control is a group of healthy subjects or non-disease subjects.

In some instances, the first reference RNA expression level is an RNA expression level obtained from the control. In some instances, the first reference RNA expression level is the RNA expression level of a set of genes comprising fibrotic markers. In some instances, the first reference RNA expression level is an RNA expression level of a set of genes comprising COL3A1, COL1A1, ACTA2, POSTN, FN1, FAP, CTHRC1, THBS1, THBS2, or combination thereof.

In some instances, the second reference RNA expression level is an RNA expression level obtained from the control. In some instances, the second reference RNA expression level is the RNA expression level of the one or more pulmonary-specific lncRNAs. In some instances, the one or more pulmonary-specific lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +).

In some instances, the method further comprises administering to the subject the modulator or the pharmaceutical composition described herein.

In some aspects, the subject in need of treatment has or is suspected to have IPF. In some aspects, the subject in need of treatment is diagnosed with IPF using high resolution computed tomography (HRCT) chest scan.

In some aspects, the measuring comprises using S1 nuclease protection assay, microarray analysis, polymerase chain reaction (PCR), hybridization technologies, reverse transcriptase polymerase chain reaction (RT-PCR), RNA-Seq, Northern blot, serial analysis of gene expression (SAGE), immunoassay, and/or mass spectrometry. In some aspects, the measuring RNA expression comprises using microarray analysis, polymerase chain reaction (PCR), hybridization technologies, reverse transcriptase polymerase chain reaction (RT-PCR), or RNA-Seq.

### Definitions

The term “noncoding RNA” as used herein, can refer to RNA species that are not translated into protein. The term “long noncoding RNA” or “lncRNA” as used herein, refers to a noncoding RNA that is at least 100, at least 1000, at least 10000, or at least 100000 nucleotides long. In some instances, “long noncoding RNA” or “lncRNA” comprises at least 2, at least 5, at least 10, at least 50, at least 100, at least 150, at least 200, at least 250, at least 300, or at least 320 exons. The total number of exons in a lncRNA or noncoding RNA is dependent upon the mapping of the specific isoform of that transcript.

The term “healthy cell” as used herein refers to a healthy cell of a healthy individual. In some instances, the term “healthy cell” as used herein refers to a cell of the corresponding tissues (e.g., lung) from a healthy individual or an individual that has not been or is not affected by a diseased condition or symptoms (e.g., pulmonary fibrosis, IPF). In some instances, it refers to a cell of the same subject but before contracting a disorder (e.g., pulmonary fibrosis, IPF) or a symptom thereof (one or more symptoms of pulmonary fibrosis, IPF).

The term “normal tissues” as used herein refers to healthy tissues obtained from a healthy individual. In some instances, the term “normal tissues” as used herein refers to corresponding tissues (e.g., lung tissue) from a healthy individual or an individual that has not been or is not affected by a diseased condition or symptoms (e.g., pulmonary fibrosis, IPF). In some instances, it refers to a tissue of the same subject but before contracting a disorder (e.g., pulmonary fibrosis, IPF) or a symptom thereof (one or more symptoms of pulmonary fibrosis, IPF.

The term “nucleic acid editing or modifying moiety,” as used herein, can refer to a nucleic acid modifying moiety that edits or cleaves the target nucleic acid. It can also refer to a nucleic acid modifying tool that suppresses the transcription of the target nucleic acid.

The term “nucleic acid analogue,” as used herein, can refer to compounds which are analogous (structurally similar) to naturally occurring nucleic acid (see, e.g., Freier & Altmann; Nucl. Acid. Res., 1997, 25, 4429-4443 and Uhlmann; Curr. Opinion in Drug Development, 2000, 3 (2), 293-213), and examples of suitable nucleic acid analogues are provided by WO2007031091, which are hereby incorporated by reference.

The term “GapmeR” is a chimeric nucleic acid molecule that contains a central sequence of phosphorothioate DNA nucleotides (“DNA gap”) flanked by sequences of modified RNA residues at either end to protect the DNA gap from nuclease degradation, whereas the central DNA gap region allows RNase-H-mediated cleavage of the target RNA. GapmeR has an internal region having a plurality of nucleosides which is capable of recruiting RNase H activity, such as RNaseH, which region is positioned between external wings at either end (5′ or 3′ wing), having one or more nucleosides. Thus, in some instances, the nucleosides within the internal region are chemically distinct from the nucleoside or nucleosides within the external wings.

The term “MixmeR” refers to a chimeric nucleic acid molecule that contains natural nucleotides and modified nucleotides, e.g., nucleotide analogues. MixmeR can comprise modified nucleotides at internal region and/or at either end of the chimeric nucleic acid molecule.

A “locked nucleic acid” or “LNA” is often referred to as inaccessible RNA and is a modified RNA nucleobase. The ribose moiety of an LNA nucleobase is modified with an extra bridge connecting the 2′ oxygen and 4′ carbon. An LNA oligonucleotide offers substantially increased affinity for its complementary strand, compared to traditional DNA or RNA oligonucleotides.

The term “methoxyethyl” or “MOE” refer to a type of modification of ribose structure. In some instances, the ribose 2′—OH group is modified and is replaced with, for example, a 2′-ethylene glycol derivative, which is also known as 2′-O-Methoxyethyl(2′-MOE) modification. An oligonucleotide with MOE modification offers higher affinity and specificity for RNA substrates, as well as greater stability to nucleases compared to an unmodified oligonucleotide.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers to the primary structure of the molecule. Thus, the term includes triple-, double- and single-stranded DNA, as well as triple-, double- and single-stranded RNA. In some aspects, it also includes modifications, such as by methylation and/or by capping, and unmodified forms of the polynucleotide. More particularly, the terms “polynucleotide,” “oligonucleotide,” “nucleic acid” and “nucleic acid molecule” comprise polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Vials, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, RNA, double- and single-stranded DNA, as well as double- and single-stranded RNA, microRNA, DNA: RNA hybrids, and hybrids between PNAs and DNA or RNA, and also include known types of modifications, for example, labels which are known in the art, methylation, “caps,” substitution of one or more of the naturally occurring nucleotides with an analog (e.g., 2′-aminoadenosine, 2′-thiothymidine, inosine, pyrrolo-pyrimidine, 3′-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2′-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30:1911-1918.

The terms “siRNA” and “short interfering RNA” are interchangeable and refer to single-stranded or double-stranded RNA molecules that are capable of inducing RNA interference. In some aspects, siRNA molecules have a duplex region that is between 18 and 30 nucleotides in length.

The terms “microRNA,” “miRNA,” and MiR” are interchangeable and refer to endogenous or artificial non-coding RNAs that are capable of regulating gene expression. It is believed that miRNAs function via RNA interference.

The terms “piRNA” and “Piwi-interacting RNA” are interchangeable and refer to a class of small RNAs involved in gene silencing. In some instances, piRNA molecules are between 26 and 31 nucleotides in length.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against one or more lncRNAs of the disclosure.

The term “complementary” and “complementarity” are interchangeable and refer to the ability of polynucleotides to form nucleotides with one another. In some instances, nucleotides are formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

As use here in, in some instances, the terms “transcribe” or “align” are used interchangeably, to refer to the sequence information of lncRNAs relative to human genome sequence information, e.g., NCBI Homo sapiens (human) GRCh38.p14 (GCF_000001405.40), from start to end locations located on the respective chromosome with respective strand.

The term “administering”, as it applies in the present disclosure, refers to contact of a modulator of one or more lncRNAs of the disclosure, to the subject. Administering a nucleic acid, such as a microRNA, siRNA, piRNA, snRNA, or antisense nucleic acid (e.g., antisense oligonucleotide or ASO), to a cell comprises transducing, transfecting, electroporating, translocating, fusing, phagocytosing, shooting or ballistic methods, or any means by which a nucleic acid can be transported across a cell membrane.

The term “pharmaceutically acceptable excipient or carrier” refers to an excipient that may optionally be included in the compositions of the disclosure and that causes no significant adverse toxicological effects to the patient.

The term “pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts comprising pharmaceutically acceptable cations include, but are not limited to, sodium, potassium, calcium, aluminum, lithium, and ammonium (including substituted ammonium).

The term “effective amount” of modulator of one or more lncRNAs of the disclosure (e.g., microRNA, siRNA, piRNA, snRNA, antisense nucleic acid, e.g., antisense oligonucleotide (ASO), ribozyme, or small molecule inhibitor, CRISPRs etc.) is an amount sufficient to effect beneficial or desired results, such as an amount that inhibits the activity of a lncRNA, for example by interfering with transcription. An effective amount can be administered in one or more administrations, applications, or dosages.

By “therapeutically effective dose or amount” of a modulator of one or more lncRNAs of the disclosure is intended an amount that, when administered as described herein, brings about a positive therapeutic response. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Alternatively, homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

As used herein, a “sample” refers to a sample of tissue or fluid isolated from a subject, including but not limited to, for example, urine, blood, plasma, serum, fecal matter, bone marrow, bile, spinal fluid, lymph fluid, samples of the skin, external secretions of the skin, respiratory, intestinal, and genitourinary tracts, tears, saliva, milk, blood cells, organs, biopsies, and also samples comprising cells or tissues derived from the subject and grown in culture, and in vitro cell culture constituents, including but not limited to, conditioned media resulting from the growth of cells and tissues in culture, recombinant cells, stem cells, and cell components.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

“Diagnosis” as used herein generally includes determination as to whether a subject is likely affected by a given disease, disorder or dysfunction of the disclosure. The skilled artisan often makes a diagnosis on the basis of one or more diagnostic indicators (e.g., a biomarker), the presence, absence, or amount of which is indicative of the presence or absence of the disease, disorder or dysfunction.

The term “comprising” (and related terms such as “comprise” or “comprises” or “having” or “including”) is not intended to exclude that in other certain instances, for example, an instance of any composition of matter, composition, method, or process, or the like, described herein, “consist of” or “consist essentially of” the described features.

The term “subject” or “patient,” as used herein, generally encompasses organisms such as mammals. Examples of mammals include, but are not limited to, any member of the mammalian class: humans, non-human primates, such as chimpanzees, and other apes and monkey species; farm animals, such as cattle, horses, sheep, goats, swine; domestic animals, such as rabbits, dogs, and cats; laboratory animals, including rodents, such as rats, mice and guinea pigs, and the like. In one aspect, the mammal is a human.

The term “treatment” or “treating,” as used herein, are used interchangeably. These terms generally refer to an approach for obtaining beneficial or desired results including but not limited to therapeutic benefit and/or a prophylactic benefit. In some instances, the term refers to eradication of the underlying disorder being treated. In other instances, the term refers to the eradication of one or more of the physiological symptoms associated with the underlying disorder such that an improvement is observed in the patient, notwithstanding that the patient is still afflicted with the underlying disorder.

The term “alleviating” or “alleviate,” as used herein, refers to amelioration, improving, or stalling the further progression of the underlying disorder being treated. In other instances, the term refers to the amelioration, improving, or stalling the further progression of one or more of the physiological symptoms associated with the underlying disorder such that an improvement is observed in the patient, notwithstanding that the patient is still afflicted with the underlying disorder.

The term “preventing” or “prevent,” as used herein, refers to the situation where the compositions disclosed herein are administered to a patient at risk of developing a particular disease, or to a patient reporting one or more of the physiological symptoms of a disease, even though a diagnosis of this disease has not been made.

Whenever the term “at least,” “more than,” or “less than” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

The term “a,” “an” and “the” include plural referents unless the content clearly dictates otherwise. Thus, for example, reference to “a lncRNA” includes a mixture of two or more lncRNAs, and the like.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The term “about,” particularly in reference to a given quantity, is meant to encompass deviations of plus or minus ten percent.

The term “positive strand” and “negative strand” as used herein, refer to double-stranded DNA as containing a positive strand (or “plus”, “+”) and a complementary strand, which refers to as a negative strand (or “minus”, “−”). The default sequence in database alignment, e.g., NCBI, is a positive strand.

### Examples

The following examples are included for illustrative purposes only and are not intended to limit the scope of the invention. Abbreviations presented herein and in figures are as follows: IPF: Idiopathic Pulmonary Fibrosis, lncRNA: Long noncoding RNA, COPD: Chronic Obstructive Pulmonary Disease, PUFIN: Pulmonary Fibrosis Induced lncRNA, TEP: Target engagement panel, FB: Fibroblast, MyoFB: Myofibroblast, AT1: Alveolar Type 1 cell, AT2: Alveolar Type 2 cell, NHLF: Normal human lung fibroblast, EMT: Epithelial-to-Mesenchymal Transition, ASO: Anti-sense Oligonucleotide, LNA: Locked nucleic acid, NT: Non-target control oligonucleotide, MoA: Mechanism of Action, scRNAseq: Single cell RNA sequencing, snRNAseq: Single nuclei RNA sequencing, GWAS: Genome wide association study, SNP: Single-nucleotide polymorphism, FDR: False discovery rate, H3K27ac: Histone 3 lysine 27 acetylation, TcMAC21: Transchromosomic Chromosome 21, Bleo: Bleomycin. All SEQ ID NOS disclosed in the application are base sequences, specific notes are added for cases where sequences are modified.

### Example 1: Multi-Omic Lung Atlas Identifies Novel Pulmonary Fibrosis Induced Non-Coding RNAs (PUFINs) as Potential Therapeutic Targets Specific to Idiopathic Pulmonary Fibrosis (IPF) Disease Pathology

In this example, multi-omic analysis were performed to investigate therapeutic targets in IPF. Using a multi-omic database mining approach, several pulmonary fibrosis induced non-coding (PUFINs) that are associated and/or specific to IPF were identified in pulmonary myofibroblasts.

### Methods:

### Lung Atlas

Transcript reconstruction was performed on 7 bulk RNAseq datasets, including GSE52463, GSE92592 and GSE99621, and 4 data sets generated in-house. Read libraries included in the analysis ranged from more than 5 billion reads. Raw RNASeq FASTQ data were cleaned/trimmed, and trimmed data were then mapped to the GRCh38 reference genome using STAR. StringTie was ran both in de novo and ab initio mode, and results were merged into a unique GTF file using Cufflinks cuffmerge. The resulting GTF was annotated using the gene symbols/names from the Gencode Reference human transcriptome (v44 GRCh38.p14). The resulting GTF was cleaned to remove suspicious transcripts using the following criteria: 1) novel mono-exonic genes were removed (annotated mono-exonic genes were kept), 2) novel genes shorter than 200 bp were removed, 3) transcripts spanning multiple annotated Genes were removed, and 4) the clean GTF was used downstream for Target ID and quantification of gene expression in all downstream analyses. Gene expression was quantified using SubRead featureCounts on each dataset separately. The aforementioned clean GTF was used as input transcriptome file. Unless otherwise specified, the expression was quantified at gene meta-feature level using transcripts as features and enabling counting of overlapping reads using the—fraction mode.

Gene expression was quantified using SubRead featureCounts on each dataset separately, and differential expression analysis was then performed with DEseq2. The differential expression results from different dataset were then aggregated with the Stouffer test.

Publicly available data from the NHGRI-EBI Catalog of human genome-wide association studies were used to annotate all lncRNAs in the GTF that contained single-nucleotide polymorphisms (SNPs) significantly associated with any of the 102 traits that were considered relevant to lung function.

### ScRNAseq Data Processing and Label Transfer

Raw FASTQ files were mapped to the human genome (GRCh38) using CellRanger (10× genomics version 7.1.0) with annotation for Gencode genes (v43) and newly discovered lncRNAs. Subsequent data analysis was performed, and the data processing was achieved by bounding the analysis to published cell barcodes and cell identities (each cell identity can be identified via a barcode provided in the annotation tables reported in the publications). Raw UMI counts were normalized with a scale factor of 10,000 UMIs per cell and subsequently natural log transformed. The top 2000 most variable genes were then used to perform a PCA (with 50 principal components, PC). 30 PC were used to identify neighboring cells and to perform UMAP. Cell annotation was transferred from published data tables. Plots were generated using ggplot2 and SCpubr libraries.

Multi-Omic snRNAseg/snATACseq

pHLFs (Lonza, Basel, Swirtzerland) were cultured and either treated with control media or TGF-β (10 ng/mL) for 48 hours. 4 million cells were harvested per condition (fibroblast and myofibroblast), and 15% of the cells were taken for RNA extraction. 10 mL of swelling buffer was added to the cell pellet, it was resuspended, and incubated for 5 minutes at 4° C. The supernatant was discarded, and the lysis buffer+SUPERase was added to dissolve the pellet (5 min incubation on ice). Cells were then counted and divided 1:1 for snRNAseq and snATACseq. 30,000 cells were separated at harvest for multi-omic sequencing. Single nuclei were barcoded using the 10× chromium platform (Single Cell 3′ Reagent Kit v3, 10× Genomics, USA). Post GEM-RT clean-up was performed on barcoded cDNA. 250 million reads/sample were sequenced (NovaSeq 6000, Illumina, San Diego, USA).

### GSEA Analysis

Gene Set Enrichment Analysis (GSEA) was performed with the top upregulated and down regulated genes from the differential expression analysis between scramble control and ASO (e.g., ASO1 (TCAGATAGAGTTAGAC (SEQ ID NO: 36)) for PUFIN1, LNA6 (TGAAGTAGCCATAGCC (SEQ ID NO: 32)) or LNA9 (GTCACCACATGTTAGT (SEQ ID NO: 35)) for PUFIN7). Prior to the GSEA, differential gene expression analyses were performed using DESeq2 with default parameters, default normalization method, and no filters on raw counts, enabling the quantification of low expressed lncRNAs. The differentially expressed genes were ranked by adjusted false discovery rate (FDR), keeping either down regulated, up regulated genes, or both. The ranks were used as input for the R package ClusterProfiler for all GSEA with default parameters, against the ontology database featured in org.Hs.eg.db (Genome wide annotation for Human. R package version 3.8.2). Noteworthily, ClusterProfiler called FGSEA package was used to perform the enrichment analyses. The result tables were processed with R custom scripts, and data visualizations were generated with ggplot2.

### Network Module Analysis

Co-expression network was constructed using weighted gene co-expression network analysis (WGCNA) R package. Publicly available dataset from the Lung Tissue Research Consortium (LTRC) was analyzed for the Network Module Analysis. This Dataset (P0005) has 39 samples with two conditions IPF (n=20) and control (n=19). After filtering out genes with zero counts (expression) in at least 10 samples, 34,036 genes (Protein-coding, long non-coding RNAs and other gene types) were kept for the network analysis. Log2 transformed counts were used as an input for WGCNA. Using the soft-threshold power=7, calculated adjacency matrix and turned it into Topological Overlap Matrix (TOM) followed by dissimilarity (1-TOM) value to minimize the effects of noise and spurious associations. Based on TOM, dissimilarity measure for hierarchical clustering was performed using flashClust R package. Once the information of the adjacency matrix was obtained, the data was transformed to TOM (Topological overlap matrix). The values in TOM matrix are between 0 and 1. The corresponding dissimilarity of TOM was calculated, on which “flashClust” was applied to produce hierarchical clustering tree of genes (clustering dendrogram of genes). TOM was used to better estimate “connectedness” of 2 genes.

Dynamic Tree cut approach was applied to identify co-expression modules with minimum module size of 50 genes. ModuleEigengenes (1st principal component of each module) and clustered modules which has similar expression profiles were calculated using flashClust based on height cut of 0.2 (which is corresponding to correlation of 0.8). To identify the relationship between modules and phenotypes, spearman correlation was applied between the eigengenes (1st principal component of each module) and the disease conditions.

Based on the results, 41 modules were identified, in which lncRNAs and protein-coding genes were co-expressed based on their similar expression patterns. A height cut of 0.2 was chosen, corresponding to the correlation of 0.8 to merge the modules that are similar. After merging similar modules into a unique cluster based on the cut-off chosen, 11 modules were identified.

### Target Engagement Panel

Target Engagement Panel (TEP) is used to define how to cluster ASO responsive genes. Firstly, an unbiased hierarchical agglomerative was performed. Genes in clusters that displayed a significant responsiveness to the ASO or ASOs being analyzed can be reclustered to define higher-confidence TEPs.

An initial Ward hierarchical clustering was utilized for the basis calculation to determine the gene subsets that were the most-sensitive subsets to ASO or ASOs treatment and could be considered for reclustering. After selecting the genes from the best performing initial cluster(s), a second clustering using the identical methodology was performed to narrow down the subset of genes that showed the strongest response to ASO or ASOs treatment. Genes from the most responsive clustered formed the second pass clustering were parts of the TEP.

### Results:

Identification of lncRNA Targets

Multi-omic studies with snRNAseq and snATACseq on an IPF in vitro myofibroblast model were performed to determine the epigenetic landscape of IPF and to identify potential therapeutic targets, e.g., non-coding RNA such as lncRNAs, for ASO knockdown related to IPF disease state and progression.

Further, lung atlas was developed to identify lncRNAs associated with IPF. The raw sequence files comprising fragments of lncRNAs were aligned to both strands (+ or −) of genomic DNA to define genomic sequence and/or information of lncRNAs associated with IPF, e.g., pulmonary fibrosis induced non-coding RNA. The information regarding each pulmonary fibrosis induced non-coding RNA (PUFIN) is shown in Table 17, which identified 12 lncRNAs (PUFINs). The sequence of each of PUFIN is transcribed from their respective strand of genomic sequence of respective chromosome with start and end locations, as provided in Table 17. Genomic regions in Table 17 are defined relative to NCBI Homo sapiens (human) GRCh38.p14 (GCF_000001405.40).

TABLE 17
Identified pulmonary fibrosis induced non-coding
RNA (PUFINs).

| | | Chromo- | Start | End |
|---|---|---|---|---|
| Name | Strand | some | location | location |
| PUFIN1 | + | chr21 | 42009177 | 42055325 |
| PUFIN2 | − | chr15 | 38864806 | 39427195 |
| PUFIN3 | − | chr15 | 38871616 | 38880364 |
| PUFIN4 | − | chr6 | 169171312 | 169188635 |
| PUFIN5 | − | chr4 | 76758554 | 76801964 |
| PUFIN6 | + | chr17 | 50199876 | 50215922 |
| PUFIN7 | + | chr2 | 215717811 | 215721161 |
| PUFIN8 | + | chr14 | 74552181 | 74560688 |
| PUFIN9 | − | chr2 | 188966458 | 188984794 |
| PUFIN10 | + | chr11 | 1218530 | 1220242 |
| PUFIN11 | + | chr6 | 74069451 | 74690727 |
| PUFIN12 | − | chr16 | 86845338 | 87058332 |

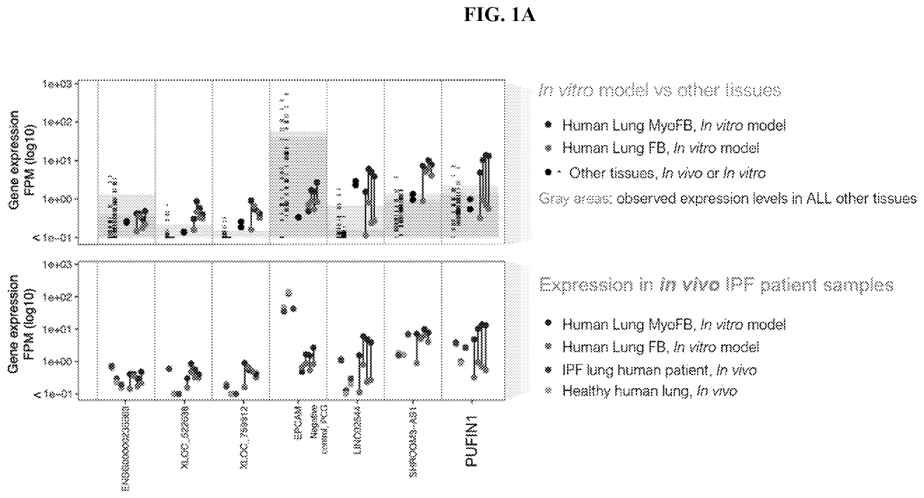

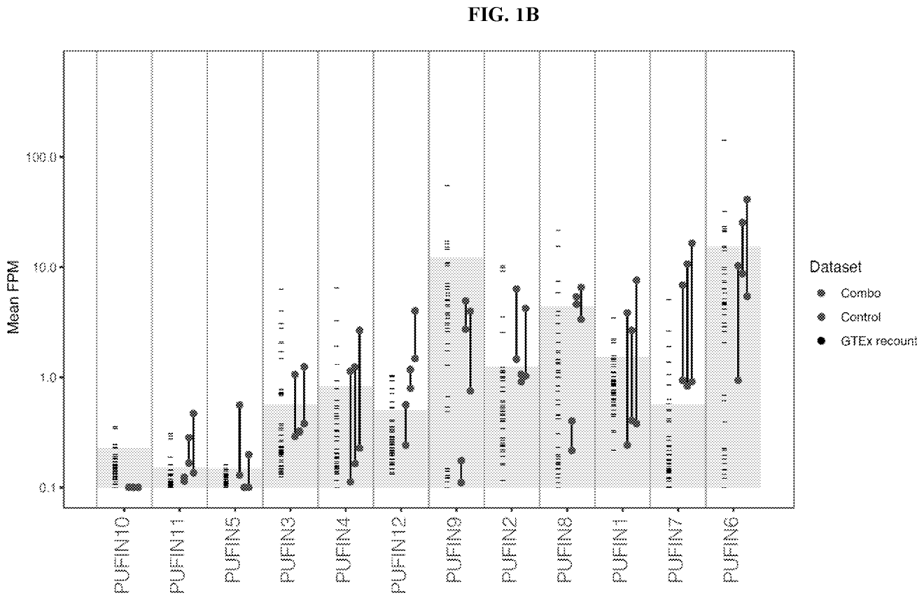

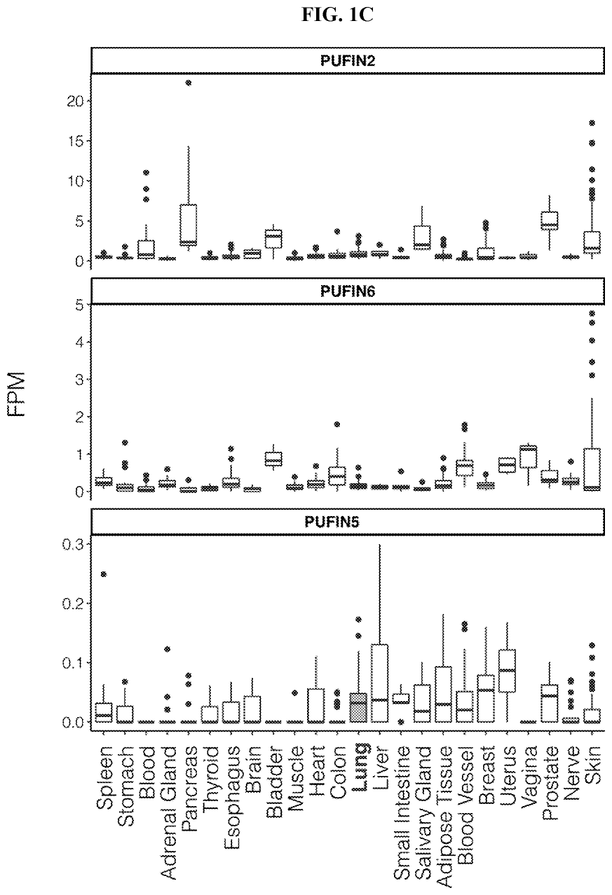

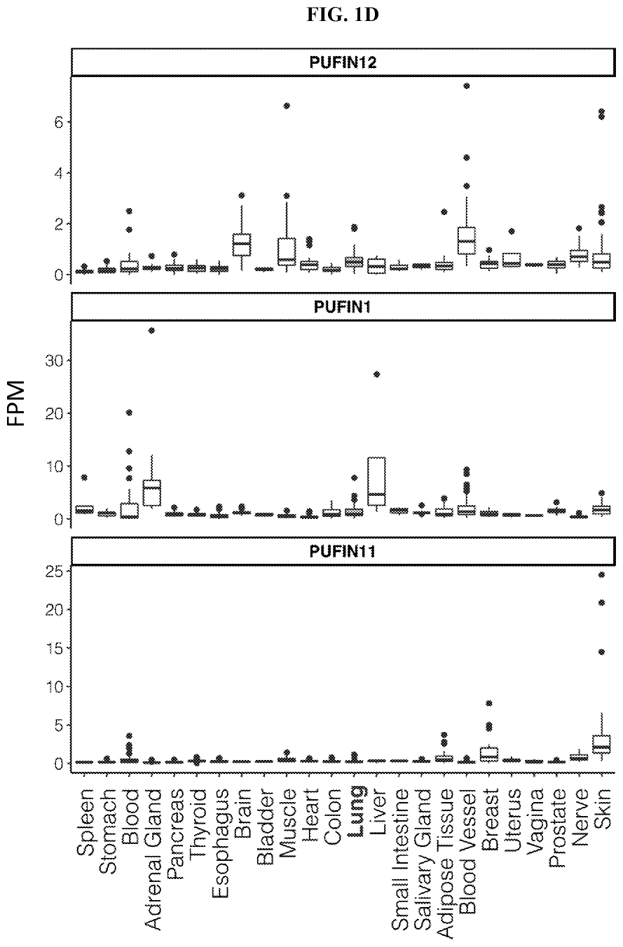

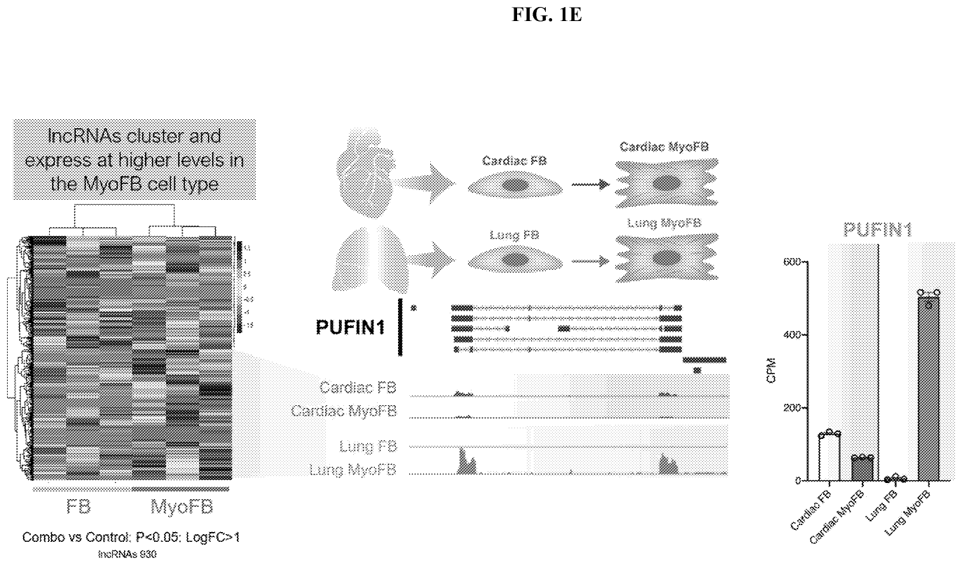

The lung atlas was developed from >100,000 noncoding RNA to identify lncRNAs (PUFIN) associated with disease. The model prioritized identifying novel, tissue-specific, cell state-specific, and disease-state specific lncRNAs. The workflow of new target discovery in lung based on in vivo IPF patient data, in vitro model, and in vivo pre-clinical model generated Atlas Assembly using results obtained from multi-omic studies. Searchable features, such as super enhancer landscape, tissue & cell state specificity, epigenomic regulation interference, disease & cell state differential expression analysis, human GWAS/eQTL signal, cross-species conservation, and regulation program correlation analysis, were utilized to identify PUFINs as therapeutic targets, as shown in Table 17. Antisense oligonucleotides (ASOs) were then developed and tested in lung myofibroblast (MyoFB) in both in vitro and in vivo as described in the next examples. As shown in FIG. 1A, association with and specificity to lung fibrosis of the identified PUFIN lncRNAs were evaluated by comparing in vitro PUFIN expression to healthy lung genotype-tissue expression (GTEx expression) (in vivo) and untreated fibroblasts cells (in vitro). The expression of the lncRNAs (PUFIN) in the disease model (in this case, human lung fibroblast to myofibroblast transition in vitro) and tissues from publicly available data (in this case, GTEx and ENCODE consortia repositories) were compared. The public data of expression of the lncRNAs (PUFIN) in different human tissues was used to create a reference of the basal and background expression of the lncRNAs (PUFIN) across the human body (gray are in the plot). Specifically, the gray area was the median expression across tissues+1.5 times the 25-75 inter-quantile range. Next, the expression of the lncRNAs (PUFIN) in the disease model (human lung myofibroblasts) was determined whether if the expression is outside (higher) or inside such a reference area. lncRNAs (PUFIN) outside the reference area were considered specific (good), whereas lncRNAs (PUFIN) inside the reference area were classified as non-specific, which is shown in FIG. 1A. Beyond the comparison between the in vitro disease model and in vivo tissue data, the expressions of the lncRNAs (PUFIN) against other in vitro models (e.g. cardiac fibroblasts, black dots in the example) were compared. This additional layer of analysis can be deployed if necessary.

The tissue specificity of potential lncRNAs (PUFINs) was ranked as shown in FIG. 1B. Expression of these lncRNAs (PUFIN) in various tissues and cell types were evaluated. As shown in FIGS. 1C and 1D, lncRNAs (PUFINs) were endogenously expressed in healthy lung tissue. As shown in FIG. 1E, PUFIN1 showed higher expression in lung MyoFB compared to lung FB, or cardiac FB and cardiac MyoFB. Similarly, PUFIN1, PUFIN6, and PUFIN7 also showed higher expression in lung MyoFB compared to other cell types (data not shown).

Integration of In Vivo scRNA-Seq Data from IPF Patients with In Vitro Myofibroblast Model Identified Novel lncRNAs Regulated in Myofibroblasts in IPF.

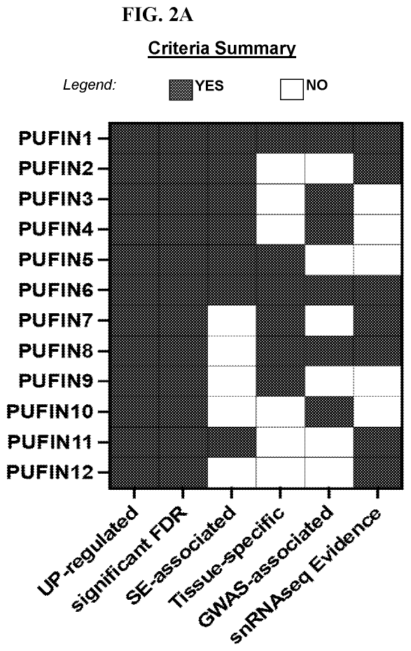

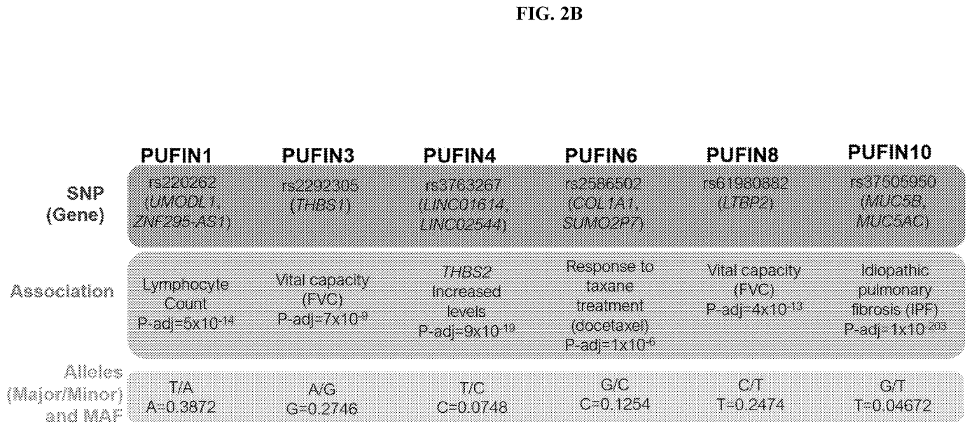

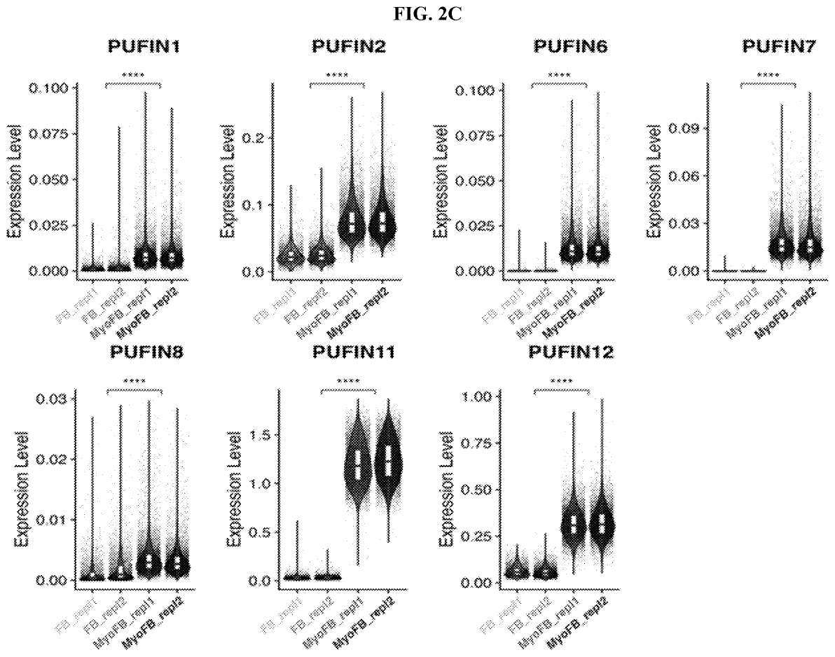

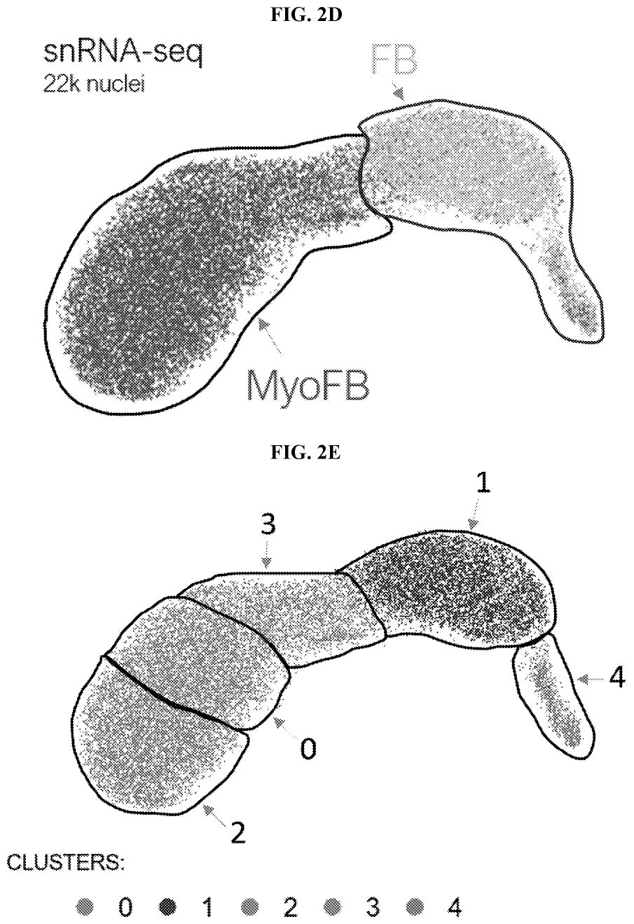

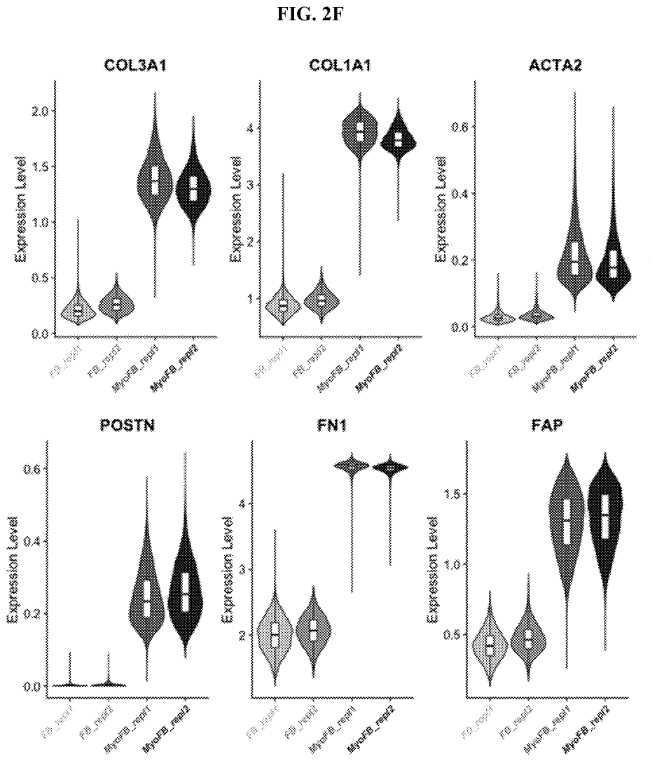

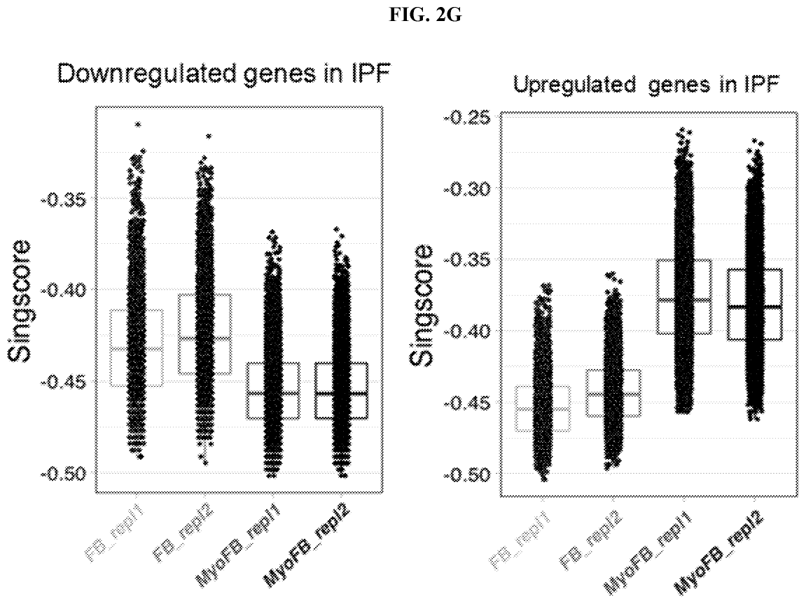

Based on the Lung Atlas, great than 39,000 lncRNAs were identified from in vivo and in house in vitro database. 4,953 of these lncRNAs were upregulated, and 712 had an FDR >0.05. Several top lncRNA targets or PUFINs with IPF therapeutic potential were identified based upon non-mutually exclusive criteria of interest: super enhancer association, tissue-specificity, human GWAS evidence, cell-type specificity, and cross-species conservation, which are shown in FIGS. 2A-2B, 1A, 1B, 1C. Super enhancers association ranked by K27Ac signal was detected in several PUFINs (data not shown). Based on snRNAseq results from ~22 k nuclei that passed quality control, cells were distinctly grouped into myofibroblast (MyoFB) cluster or fibroblast (FB) cluster (data not shown). MyoFB cluster was correlated with high density of ACTA2 and POSTN, which are MyoFB markers (data not shown). Further, as shown in FIG. 2C, lncRNAs (PUFIN) showed significantly higher expression in the myofibroblast cluster (MyoFB) compared to the fibroblast cluster (FB).

Further, based on snRNAseq results, ~22 k nuclei passed quality control and were clustered into two larger clusters (FB and MyoFB) by location of key fibrotic gene expression markers (ACTA2, COL1A1, COL3A1, POSTN, FAP, FN1) that define the MyoFB cluster (FIG. 2D). Upon additional clustering, these two larger clusters (FB and MyoFB) were further divided into five clusters (undirected clustering) (FIG. 2E). Among the five clusters (clusters 0, 1, 2, 3, and 4), cluster 0 and cluster 2 had the most MyoFB population (data not shown). 51 genes were differentially expressed (FC>1, p-adjusted <0.05) between the five clusters, and these clusters were generated using Cell Ranger and undirected clustering. 15 genes (COL4A2, CALD1, CCN2, DDAH1, COL4A1, PLCB4, ATP10A, IGFBP7, LINC00578, PAWR, SERPINE1, MYL9, DMD, NEGRI, TAGLN) were upregulated in cluster 2, which were classified as the primary myofibroblast cluster. The undirected clustering of genes into a primary myofibroblast cluster (cluster 2) supported the rationale for a direct clustering based upon canonical fibroblast gene expression (data not shown). As shown in FIG. 2F, two biological replicates of myofibroblasts showed highly differential expression of canonical myofibroblast genes (COL3A1, COL1A1, ACTA2, POSTN, FN1, FAP). FIG. 2G shows the corrected and uncorrected IPF gene score (Singscore) in fibroblast vs myofibroblast clusters. As shown in FIG. 2G top panel, differentially expression of upregulated genes in IPF were upregulated in MyoFB cluster compared to the FB cluster. Similarly, as shown in FIG. 2G bottom panel, differentially expression of downregulated genes in IPF were downregulated in MyoFB cluster compared to FB cluster. Further, 385 genes were upregulated in the myofibroblast cluster (FC>1, p-adjusted <0.05) compared to the fibroblast cluster (data not shown). Examples of genes that were upregulated in MyoFB compared to FB are KIF26B, COL1A1, SERPINE2, COL1A2, ADAM12, SPOCK1, ITGBL1, COL8A1, FN1, and TPM1. Examples of genes that were downregulated in MyoFB compared to FB are ABCC1, NCKAP5, FOSL1, SDK1, CACNAIA, ANO1, RNA5-8SN1, SMAD3, SHANK2, and ENSG00000229771.

Myofibroblast and fibroblast replicated cluster distinctly in the snATACseq data, and expression of PUFINs (PUFIN1, PUFIN2, PUFIN3, PUFIN6, PUFIN7, PUFIN8, PUFIN11, PUFIN12) was detected preferentially in the MyoFB cluster in the snATACseq dataset (data not shown). snATAC-seq cell-state phenotyping also showed that chromatin encompassing genomic loci of PUFINs became accessible upon myofibroblast activation. Higher chromatin availability indicated that there was higher availability for transcription at that site (data not shown). Increased chromatin accessibility (open regions) indicated that the loci encoding for PUFINs became more active in myofibroblasts. Also, changes in accessibility levels at loci encoding for PUFINs suggested that PUFINs play a role in governing myofibroblast cell-state transition. 8831 differentially expressed peaks were identified between fibroblast and myofibroblast cell populations.

Further, during cell differentiation from fibroblasts to myofibroblasts, changes in gene expressions were observed (data not shown). Chromatin accessibility of unique and differential peaks revealed a cell state that indicated a drive to myofibroblast activation. Chromatin accessibility reveals unique and differential peaks of gene cluster in MyoFB. Motif analysis on snATACseq peaks also allowed identification of transcription factors regulating fibroblast-to-myofibroblast transition. From motif enrichment analysis, transcription factors enriched in fibroblast-specific peaks (close in myofibroblasts) were JUNB, FOS, JUN, BACH1, FOSB, FOSL1, FOSL2, JUND, and BACH2. On the other hands, RUNX1, RUNX2, TEAD4, SMAD3, SMAD4, SMAD9, TEAD1, and CBFB were part of an upregulated transcription factors enriched in myofibroblast-specific peaks (open in the myofibroblast state) and associated with myofibroblast specific peaks.

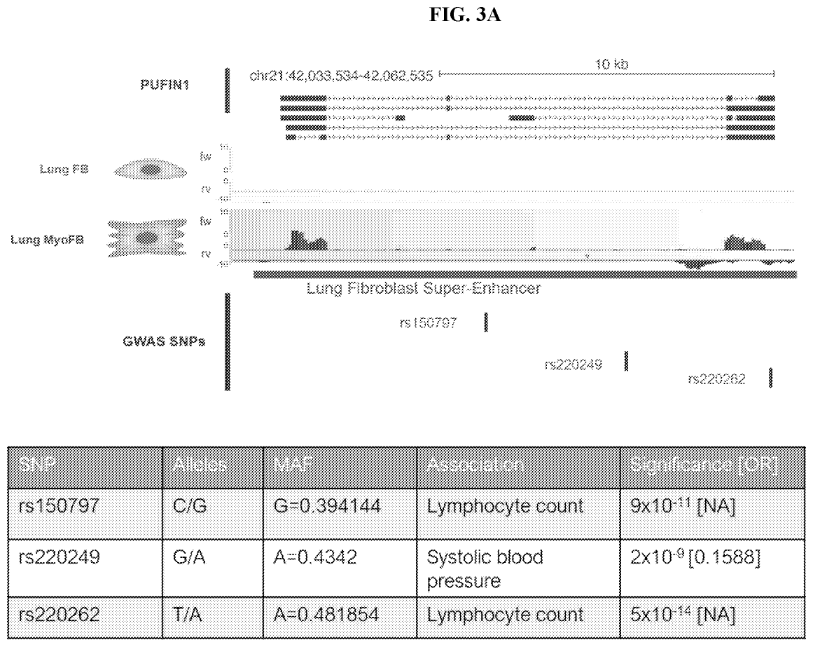

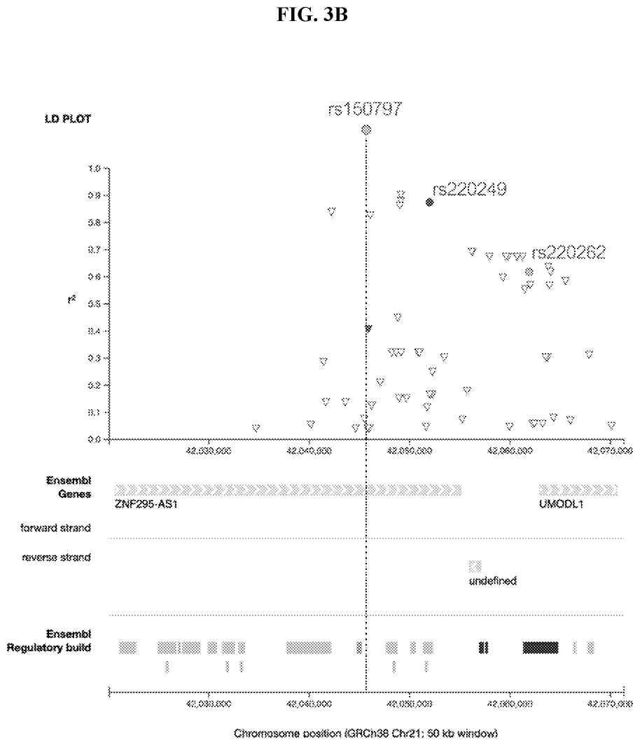

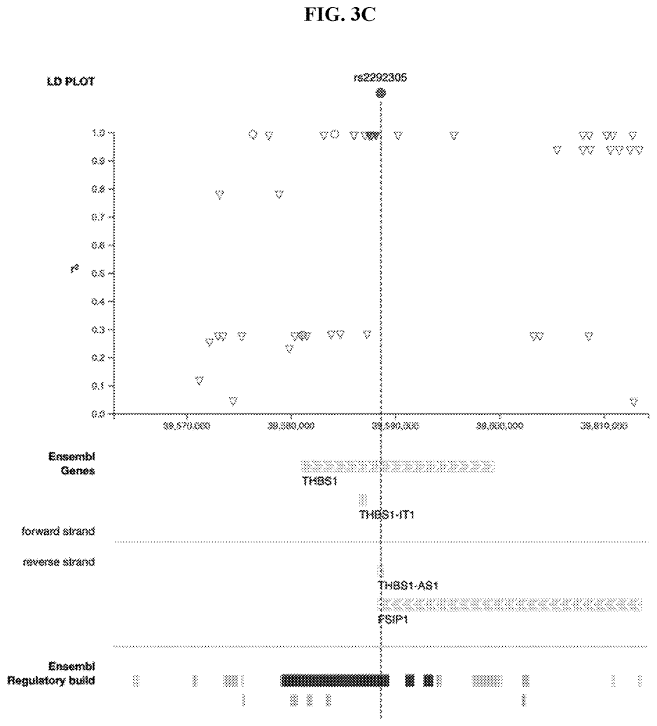

Publicly available data from the NHGRI-EBI Catalog of human genome-wide association studies was used to annotate all lncRNAs in the GTF that contained single-nucleotide polymorphisms (SNPs) significantly associated with any of the 102 traits that were considered relevant to lung function. All the terms considered in the analysis are available in Table 18. Several PUFINs have human genome wide associated SNPs (GWAS) hits on their transcripts. As shown in FIG. 3A, in PUFIN1, there were three significant GWAS alleles on the PUFIN1 transcript, and two of these (rs150797 and rs220262) SNPs were associated with lymphocyte count. As shown in FIG. 3B, in LD plot of these SNPs showed rs150797 as having the strongest association on the PUFIN1 transcript. As shown in FIG. 3C, on the THBS1 transcript, there was a SNP (rs2292305) that had significant association with decreased FVC lung function. PUFIN2 and PUFIN3 might both affect THBS1 expression or interact with this significant GWAS site. The closest upstream and downstream protein coding gene in the genome were investigated. In the downstream analyses, the expression of the neighboring genes that were affected upon target (PUFIN2/3) knock-down was examined.

Novel lncRNAs (PUFIN) were Specific to the IPF Disease State.

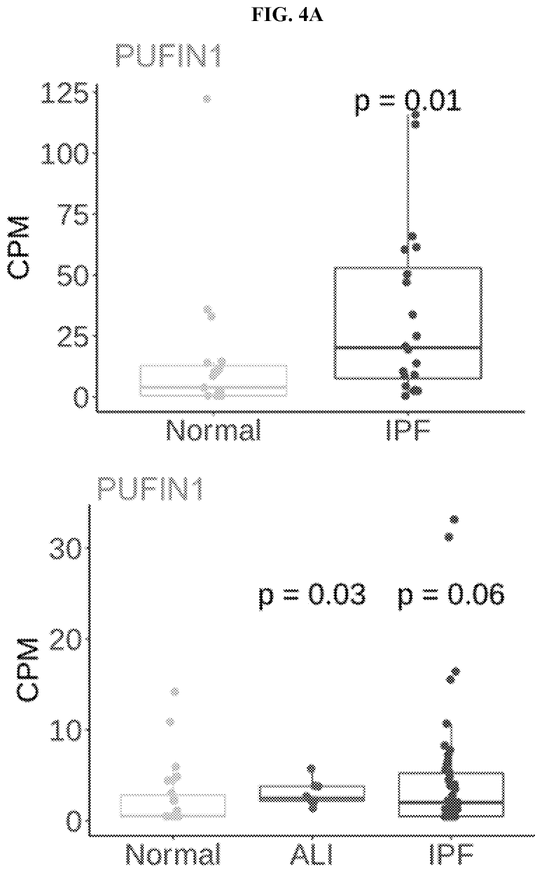

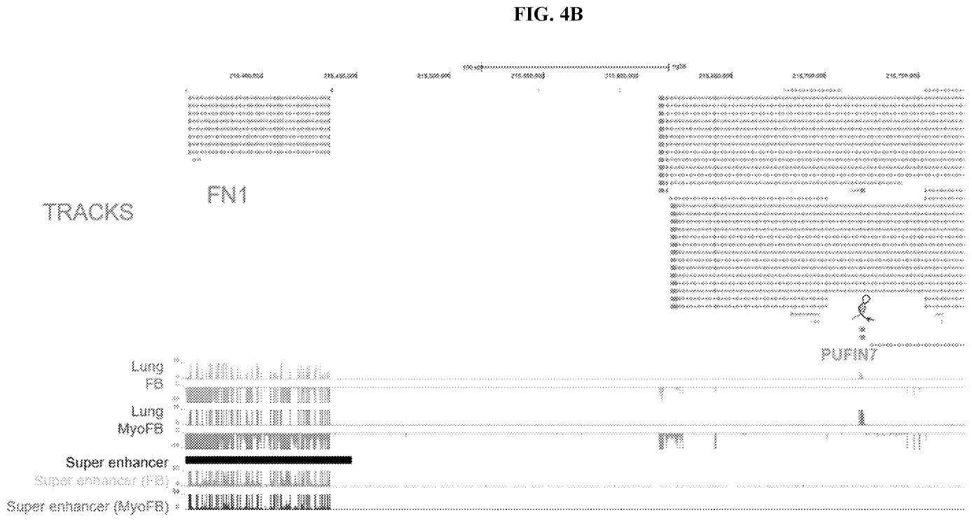

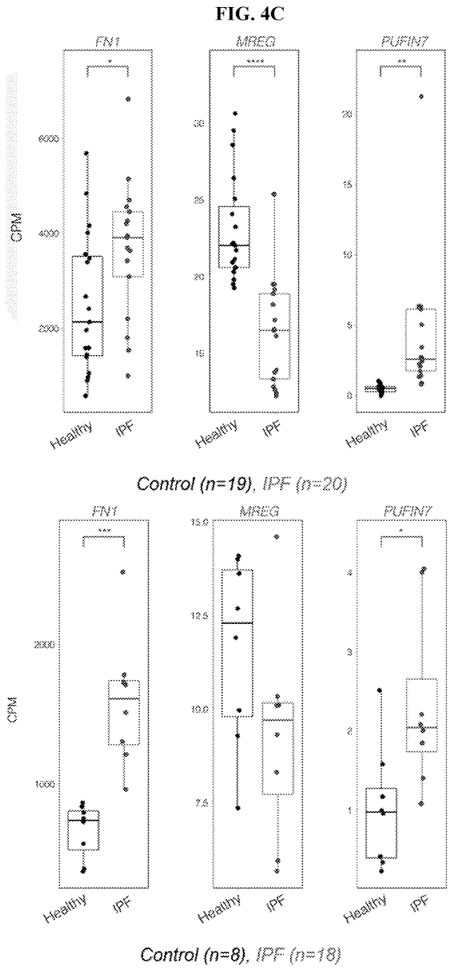

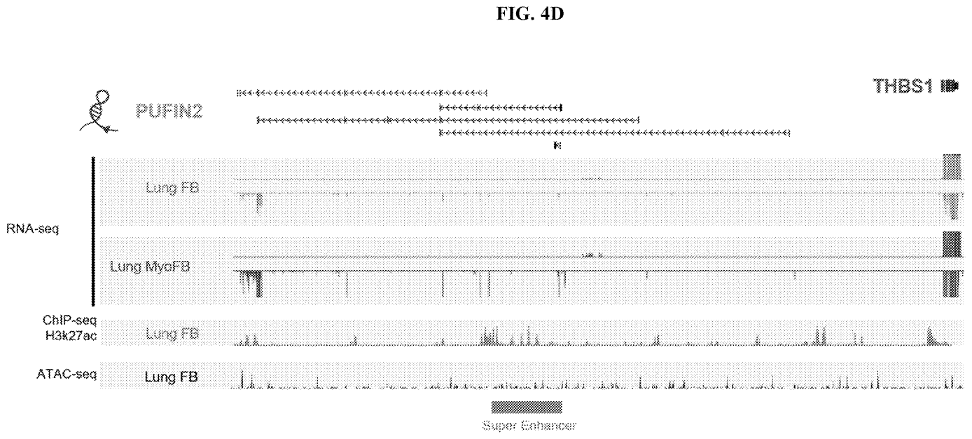

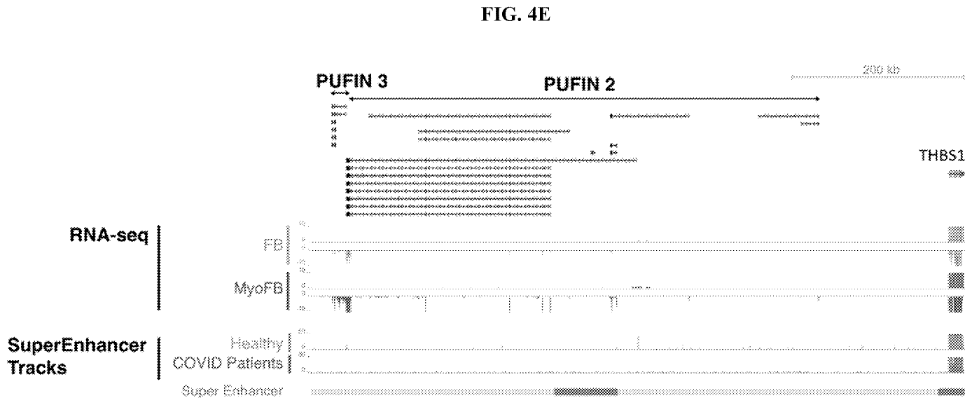

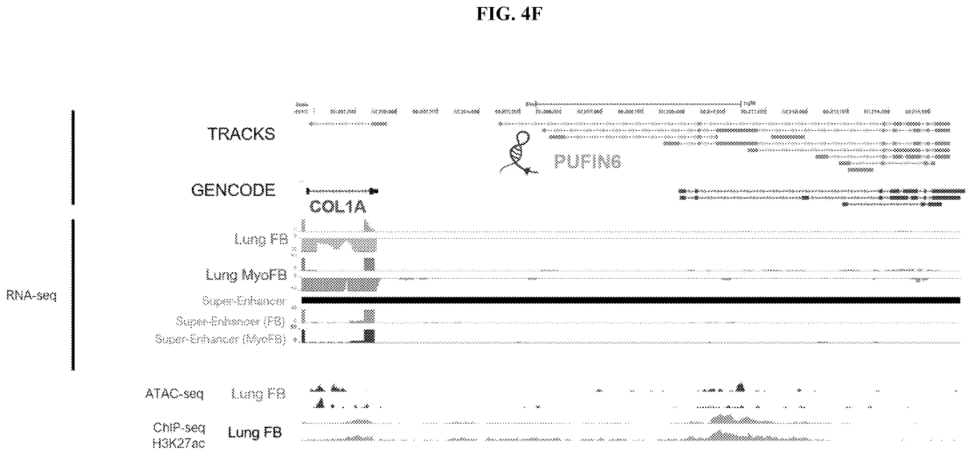

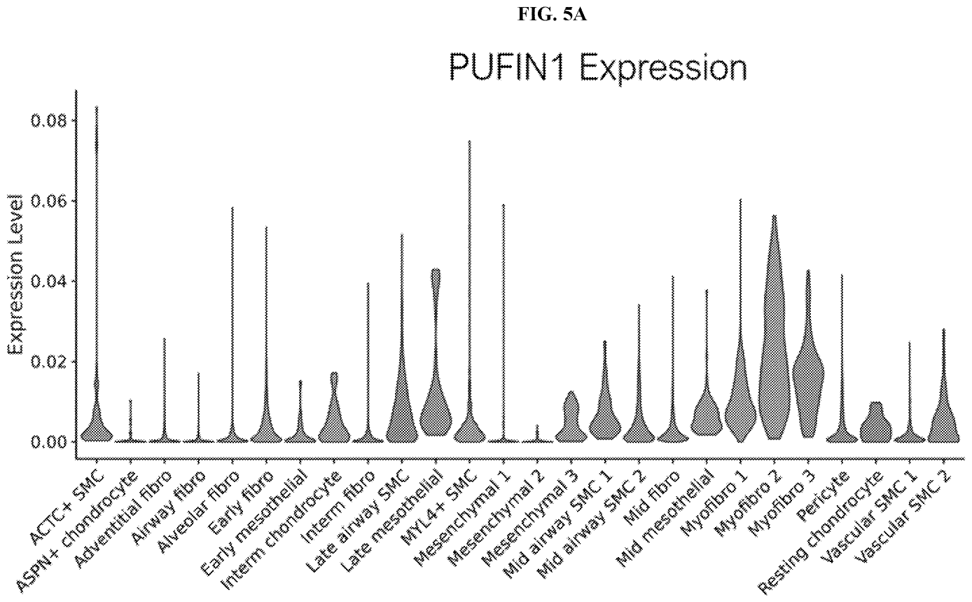

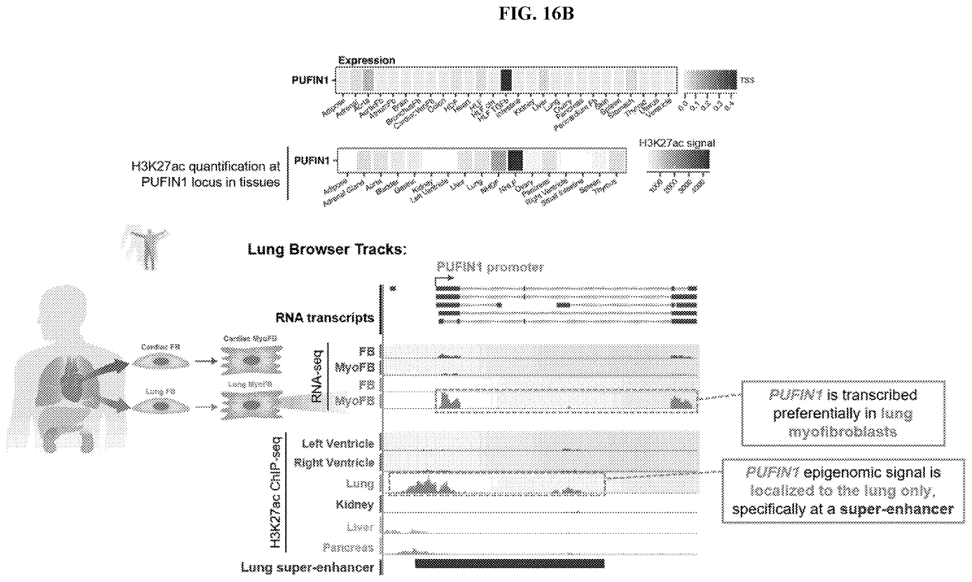

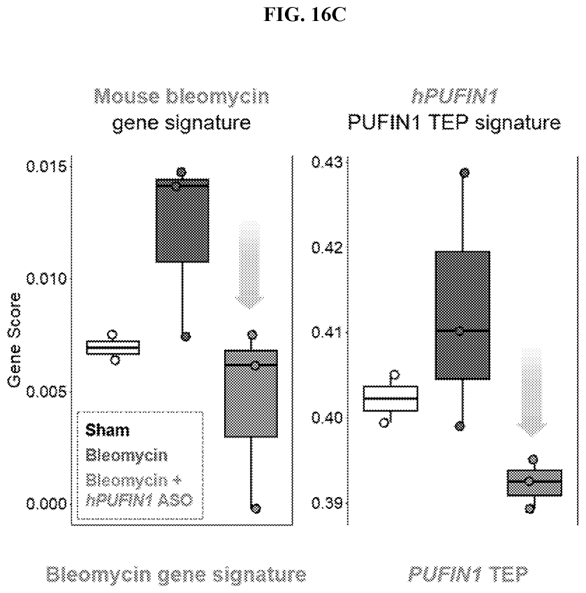

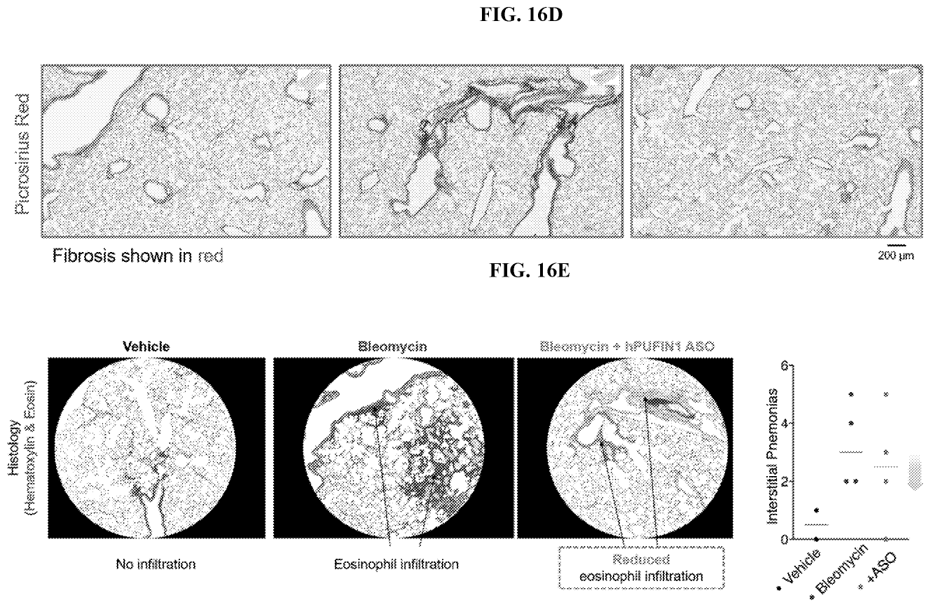

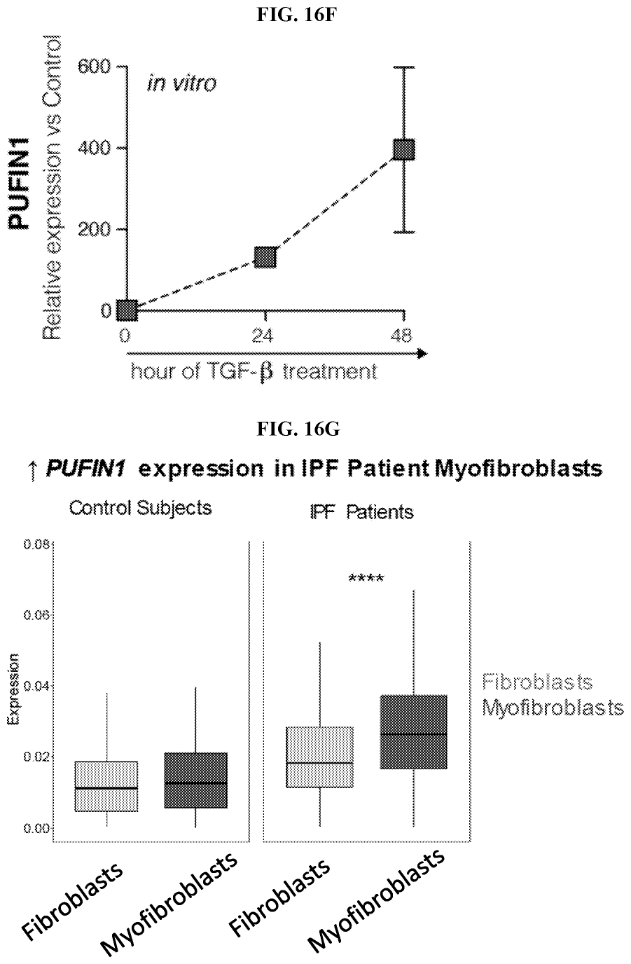

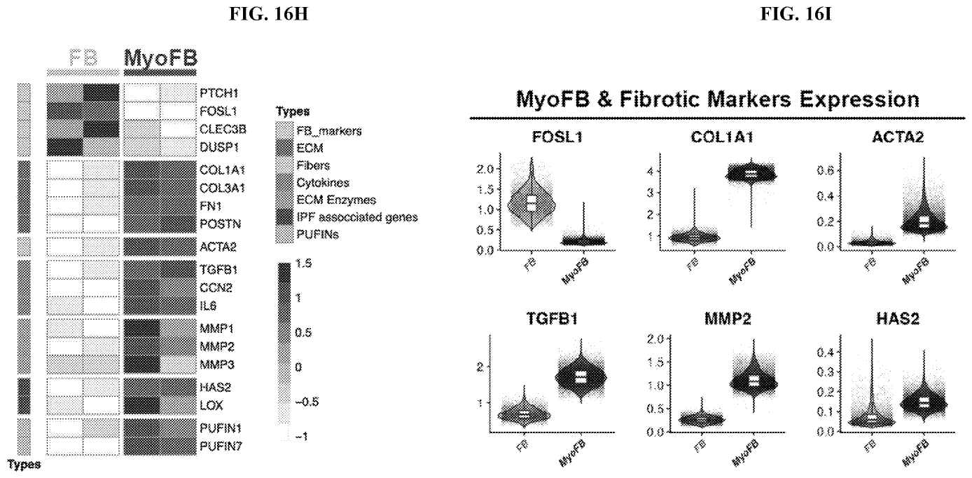

Expression levels of several lncRNAs (PUFINs) was examined using on bulk RNA-seq data. As shown in FIG. 4A, several PUFINs showed significantly increase expression in lung tissue samples derived from patients with IPF compared to lung tissue samples derived from control subjects (data in the top panel is analyzed using the RNA-seq data in Schafer et al., Nat Commun. (2017)). Results of RNA-seq analysis of lung tissues from a cohort of patients with transplant-stage IPF, acute lung injury (ALI), and nondisease controls, also show that expression of PUFINs is increased in lung tissues derived from IPF patients (data in the bottom panel is analyzed using the RNA-seq data in Sivakumar et al. ERJ Open Research (2019)). Normal in FIG. 4A refers to nondisease controls. Similar analysis of lncRNA expression was performed in a single cell level using single-cell RNA-seq data reported by Adams et al., Sci Adv (2020). As shown in FIG. 16G, PUFIN expression was upregulated in IPF patient myofibroblasts.

Further genomic analysis identified the relative locations of PUFINs to one or more genes associated with fibrosis. For example, as shown in FIGS. 4D-4E, PUFIN2 and PUFIN3 are located upstream of THBS1. As shown in FIG. 4F, PUFIN6 is located upstream of COL1A1. As shown in FIGS. 4B-4C, an PUFIN7, is located upstream of FN1 All those PUFIN lncRNAs, as well as nearby genes associated with fibrosis (THBS1, COL1A1, FN1) showed increased expression levels in lung myofibroblast samples compared to lung fibroblast, indicating potential co-expression or regulation of expression of the fibrosis associated genes by the lncRNA.

lncRNAs (PUFIN) Showed Cell-Type, Phenotype Specificity in IPF Patients.

Several PUFINs comprising PUFIN1, PUFIN2, PUFIN3, PUFIN6, PUFIN7, PUFIN8, PUFIN11, and PUFIN12, are present in fibroblasts and airway cell populations during lung development (data not shown). Such initial tissue specific expression pattern is observed to change upon being affected by lung fibrosis (e.g., IPF). For example, during development, PUFIN1 is exclusively expressed in ciliated epithelial cells (data was obtained from Human Cell Atlas (PMID: 30917859 PMCID: PMC6437997; data not shown). In IPF patients, PUFIN1 expression decreased in ciliated cells and increased in myofibroblasts. Similarly, PUFIN7 is clustered with mesenchymal cells that expressed the HAS1+phenotype, which has been strongly linked to IPF, indicating that PUFIN7 expression is strongly associated with IPF disease state (high expression in IPF compared to COPD). Similar to PUFIN7, PUFIN3 showed strong association with disease state of IPF as it showed upregulation in abnormal basal cells that express the KRT17+ phenotype. PUFIN6 also showed higher expression in myofibroblast cells as shown by scRNAseq data in IPF patient sample (HAS1+ and mesenchymal cells). PUFIN2 showed high expression in ciliated cells and basal cells in IPF patient sample, which were associated with fibrotic processes (data not shown).

These results indicate that several PUFINs that are specifically associated with IPF in vivo were identified, and these PUFINs are strongly associated with IPF disease pathology and may play a regulatory role in cell type transition or cell stage regulation. For example, PUFIN1 may have a regulatory role in EMT due to its expression shifting from cilia to myofibroblasts in IPF. PUFIN7 was associated with fibroblasts in lung development in healthy cells, but in IPF, PUFIN7 was expressed with HAS1+fibroblasts, which is a known pathogenic population associated with IPF progression. PUFIN3 was expressed in the KRT17+abnormal basal cell population, which is another cell subphenotype that has been associated with IPF pathologies including end stage senescence.

Similar to PUFINs, groups of genes associated with IPF or IPF pathology have been identified by using a combination of meta-analysis and machine learning of in vivo and in vitro IPF patient gene expression datasets. This group of genes represents IPF gene signature comprising i) gene expression changes and ii) gene expression consistency. The IPF gene signature was validated with control, acute lung injury (ALI), and IPF datasets, and can be used to discriminate IPF from other pathologies. The IPF gene signatures were constructed and shown in Table 18. As described in this example, IPF gene signature refers to a group of genes or a subset of genes whose collective transcription significantly correlates with IPF pathology.

The IPF gene signatures obtained from in vitro experiments were further validated with IPF patient single cell RNA sequencing (scRNAseq) data (obtained from Adams, T. S. et al. Sci Adv (2020)), which disclosed a single cell RNAseq study of whole lung dissociated from IPF, chronic obstructive pulmonary disease (COPD), and control patients. The epigenetic regulation and network identified in the in vitro model using human lung fibroblast were further analyzed and validated with in vivo patient data. The human lung fibroblast network showed some transcription factors (TFs), e.g., SMAD3, RUNX1, and FOSL1, as key master regulators of fibroblasts to myofibroblast transition. The in vitro model using human lung fibroblasts as described herein recapitulated in vivo IPF transcription, showing that IPF gene signatures from IPF patients could be reproduced in vitro. For example, transcription factor analysis showed that RUNX1 and SMAD3 were upregulated in the cells affected by IPF (data not shown).

Gene signature from IPF patients was also recapitulated at single nucleus resolution from in vitro model using human lung fibroblasts treated with TGF-β. IPF patient data were leveraged to generate gene signatures from scRNAseq, and these gene signatures showed upregulation in IPF affected myofibroblasts. The gene signatures from these cells recapitulated the IPF gene signature (genes listed in Table 18) derived from public human genomic data, suggesting that the results from in vitro model correlated with subsets of genes that were upregulated or downregulated in patient fibrotic lung tissue. 427 IPF genes in IPF gene signature were upregulated in myofibroblast compared to fibroblasts, and 317 genes in IPF gene signature were downregulated in myofibroblasts compared to fibroblasts (data not shown).

Next, a transcription regulatory network (TRN) modulated or affected by the IPF model was identified and evaluated whether it replicated IPF data transcription network. Integration of snRNAseq and snATAC-seq datasets enabled unbiased reconstruction of the transcriptional regulatory network (TRN). The TRN in lung fibroblast to myofibroblast transition was performed using multi-omics in vitro models. TRN analysis identified functionally relevant gene sets regulated by each specific transcription factor (TF). A TRN revealed groups of functionally relevant genes (these were called regulons) that were regulated by key transcription factors (TFs) that drove myofibroblast activation. Quantifying regulon gene expression is a robust method to assess master TF activity. The TF signatures regulon derived from in vitro data was validated using in vivo patient data. For examples, FOSL1 transcription factor was shown to regulates 279 gene comprising BCL2L1, DNMT3A, ETV6, FGF14, IGF1, NFIA, or TCF4. These TFs were shown to be disease relevance, confirmed on independent in vivo IPF patient data. The inferred TF network regulated a significant portion of the genes in the IPF myofibroblast signature. As described in this example, the IPF myofibroblast signature refers to genes consistently upregulated or downregulated in myofibroblast in multiple independent in vivo (Adams, et al., 2020) and in vitro cohorts. The results were also validated with publicly available data as disclosed in Jones, D. et al. bioRxiv (2020), and Sueblinvong, V. et al. Am J Med Sci (2012). Further, 142 genes were found to be co-regulated by RUNX1 and SMAD3 TFs, and these genes were functionally enriched in, e.g., assembly of collagen fibrils, collagen chain trimerization, and collagen biosynthesis and formation (data not shown). Genes in PUFIN1 TEP were mainly regulated by SMAD3, RUNX1, and FOSL1 transcription factors (data not shown).

### Example 2: ASO Knockdown of PUFIN1 Attenuated Myofibroblast Gene Expression

In this example, ASO sequences were developed in silico to target and modulate expression of PUFIN1, as identified in the Example 1.

### Methods:

### ASO Designs and Generation

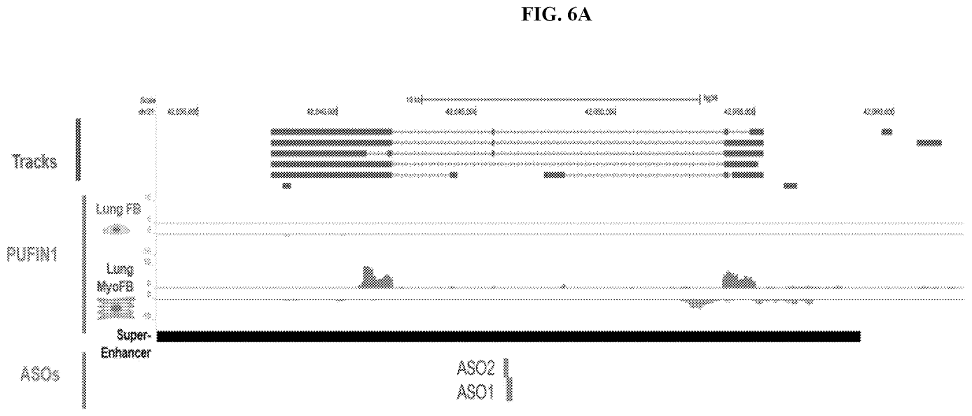

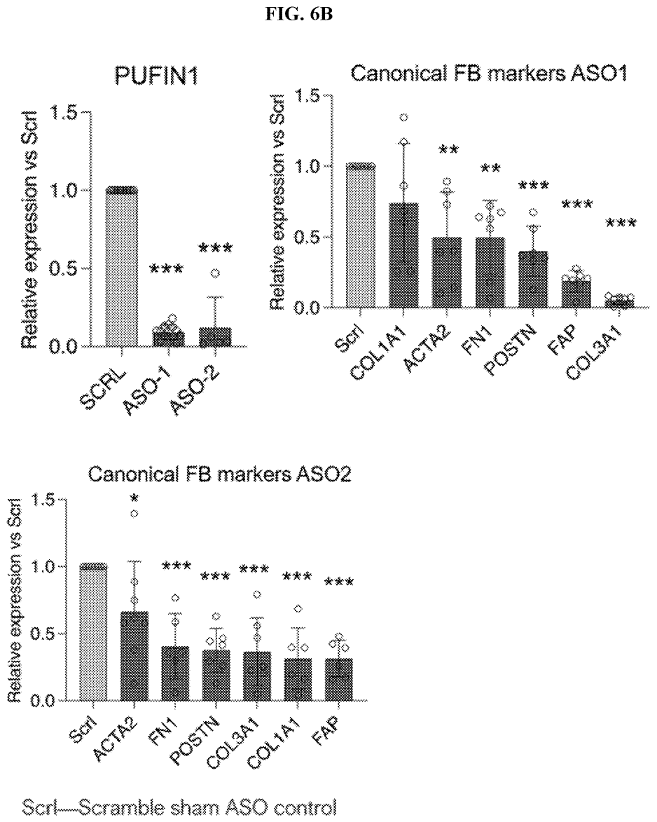

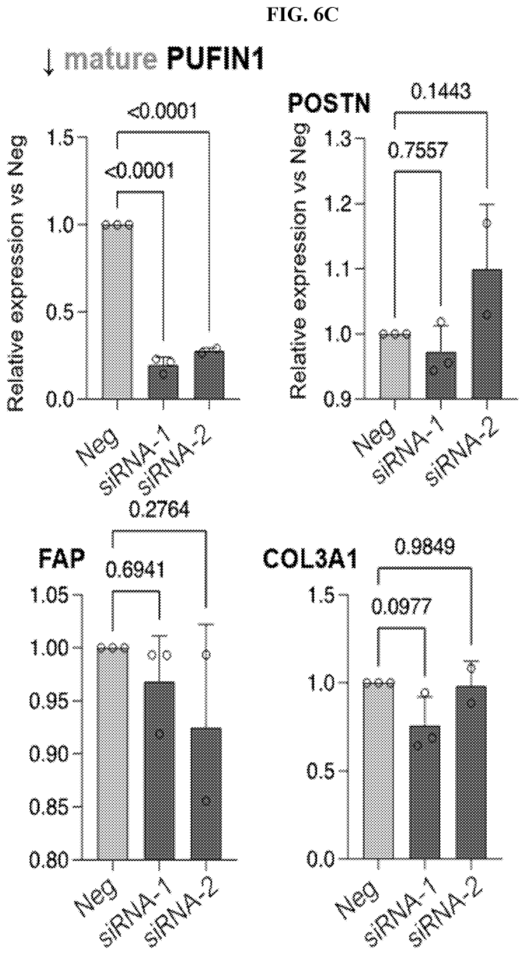

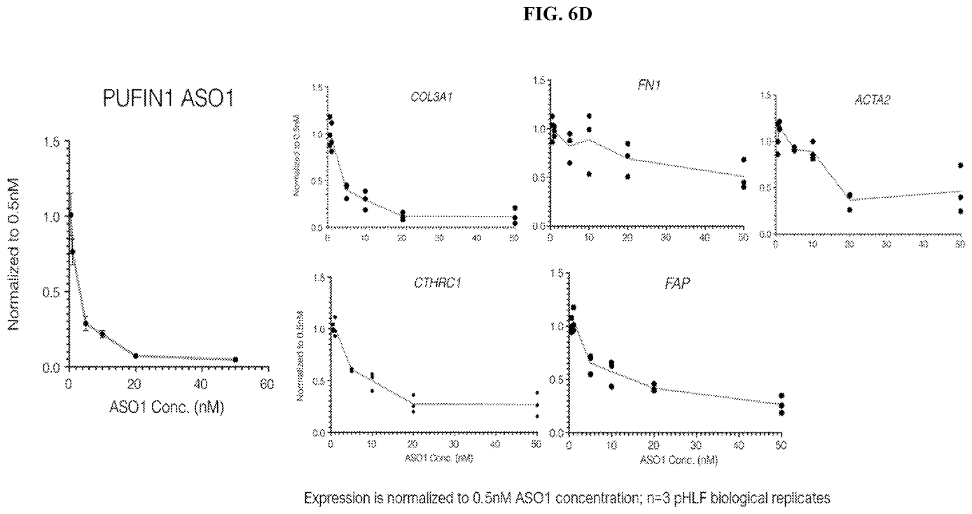

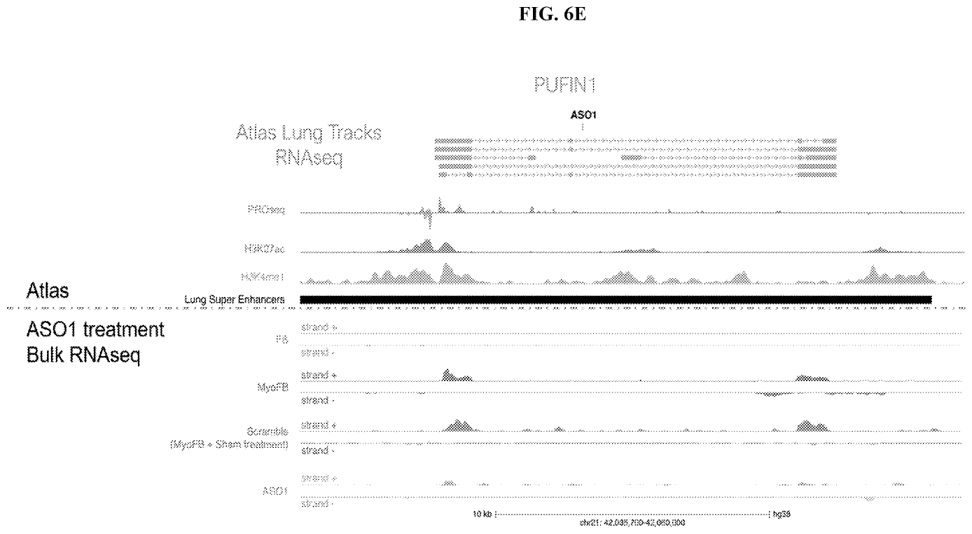

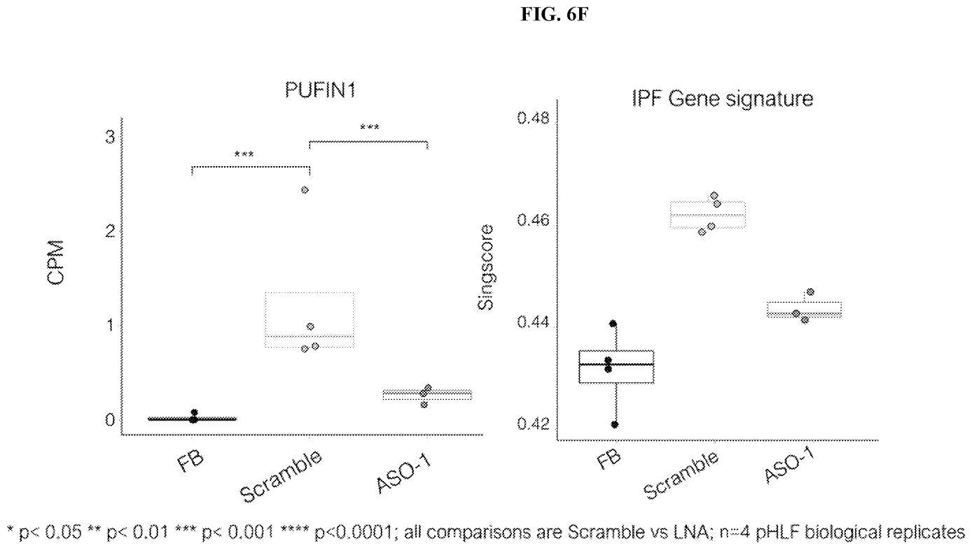

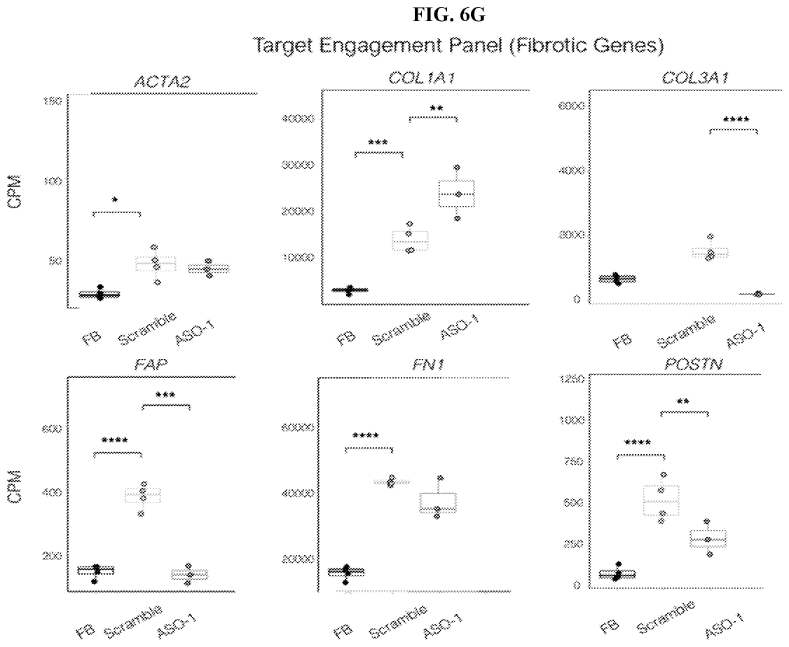

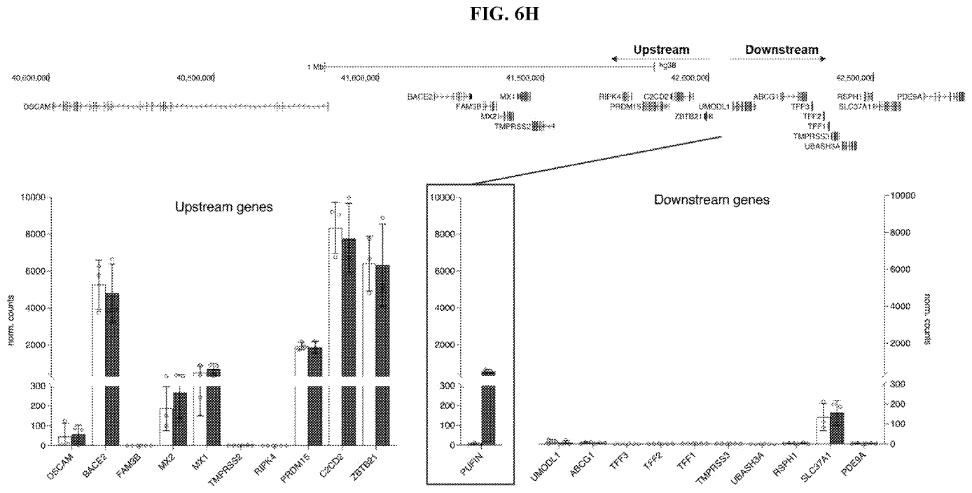

Generated ASOs comprised one of two modified oligonucleotides in its 5′- or 3′-wing regions: locked nucleic acids (LNAs) and Methoxyethyl modified nucleotides (MOEs). The in silico ASO design process generated all potential ASO sequences for a given loci (chromosome, start location, end location, strand). The ASO filtering process eliminated ASO designs that break chemical constraints (duplex energy, hybrid energy) and biological constraints (having >1 CG or a high >60 CG %) that limit ASO effectiveness in vitro and in vivo. Table 6 shows the ASO sequences generated in silico to target PUFIN1. ASO sequences targeting PUFIN1 that were selected for further testing can be found in Table 5 or Tables 25-26. Among these sequences, two ASO sequences, ASO-1 (SEQ ID NO: 36) and ASO-2 (SEQ ID NO: 37), target the super-enhancer region within the genomic locus where PUFIN1 is transcribed (FIG. 6A).

### Cell Culture

Primary human lung fibroblasts (pHLFs) were obtained from the manufacturer (Lonza, Switzerland). Cells were cultured according to the manufacturer's instructions with FGM-2 bullet kit (Lonza, MD, USA) with the addition of 1% Pen-Strep (Gibco, NY, YSA). For transfection, fibroblasts were seeded at 150,000 cells/well 24 hrs prior to ASO transfection with either ASO or scramble control (Qiagen, MD, USA) at 20 nM. All samples were processed in three biological replicates. X-treme Gene HP DNA transfection reagent (Roche, Mannheim, Germany) and reduced serum media Optimem (Gibco, NY, USA) were used to aid the transfection and the cells were left to grow for 24 hrs. Fibroblasts were then induced with 5 ng/ml TGF-β and Serum Free Lonza FGM-2 media or no treatment control for 24 hrs. Fibroblasts were then lysed on the plate using the Lysis Buffer from High Pure RNA Isolation Kit (Roche, Mannheim, Germany) and lysate was collected.

RNA Extraction and qPCR

mRNA was extracted via the High Pure RNA Isolation kit (Roche, Mannheim, Germany) according to the manufacturer's instructions. Quality and quantity of mRNA were assessed via Nanodrop One (ThemoScientific, CA, USA). cDNA was synthesized using Quantitect Reverse transcription Kit (Qiagen, Hilden, Germany). For protein coding mRNA genes, qPCR was then run on Quantstudio5 using Taqman Premix Ex Taq (Takara, Shiga, Japan) and custom TaqMan gene expression assays (Applied Biosystems, CA, USA). For lncRNA targets, TB Green Premix Ex Taq (Takara, Shiga, Japan) and primer+reverse mix at 10 μM (IDT, CA, USA) were used to access qPCR expression (Primers in Supplementary Methods). Fibrotic markers' expression was measured through relative quantification based on an internal reference gene (GAPDH) to determine fold change differences in expression of the target gene. Conversion of quantification cycle values (Cq) into normalized relative quantities was done using a single reference gene (GAPDH) and NRQ=2-AACT (Livak, K. J., & Schmittgen, T. D. (2001)).

### Bulk RNAseq

RNA quality was assessed using the Bioanalyzer 4150 TapeStation (Agilent). RIN>8 were considered high-quality samples for deep sequencing. RNAseq libraries were built according to the Illumina protocol via Poly (A) capture ligation-based addition of adapters and indexes. The final libraries were checked for quality and quantity using D1000 screen tape and Qubit. Samples were sequenced at 150 million reads/sample for discovery on NextSeq or NovaSeq platforms (Illumina). RNAseq data was preprocessed to remove background noise, normalize the data, and to filter out genes with low expression levels.

### Lung Browser Tracks for Super-Enhancer Analysis

Super-enhancer: Super-enhancer is an enhancer that it is larger in size than typical enhancers. It binds multiple transcription factors or have a greater density of potential transcription factor binding domains than typical enhancers. It has an elevated ability to activate transcription, and it is more sensitive to perturbation. Whyte, W. et al. Cell. (2013) and Lovén, J. et al. Cell. (2013). Lung super-enhancers were identified by the Rank Ordering of Super-Enhancers (ROSE) algorithm in lung fibroblasts and myofibroblasts.

### TcMAC21 Bleomycin Murine Model

Information on the lung atlas was used to determine the synteny positioning for mouse PUFIN1 (mmPUFIN1). Synteny was based upon taking the neighboring protein coding genes in human PUFIN1 (hsPUFIN1) and involved mapping the positional and functional equivalent in the mouse genome to find mmPufin1. When hsPUFIN1 was mapped to find mmPufin1, the syntenic loci for mmPufin1 was revealed to be split between two mouse chromosomes (chr16 and chr17). HsPUFIN1 seems to be a site of recombination and is not conserved in small mammals (rat, murine, rabbit, etc.). Initially, studies were done in mouse lung fibroblasts (MLFs) in vitro with the rationale that the partial transcript on chr16 with the downstream genes of the target could prove to be a functional equivalent (mmPufin1) in a murine model. These studies revealed that the truncated mmPufin1 on chr16 did not have the same functionality that hsPUFIN1 had shown in vitro. Due to this, a humanized murine model where ASO1 (TCAGATAGAGTTAGAC (SEQ ID NO: 36)) could be directly tested against hsPUFIN1 was sought. The transgenic mouse line B6D2F1 was used to breed the transchromosomic (Tc) mouse line that fully expressed the human chr21 (HSA21). This transchromosomic mouse model (TcMAC21) was bred for and exclusively used for the study of trisomy. For study of IPF pathology using the animal models, the intratracheal bleomycin model is the most well-accepted and well-recognized pharmaceutical murine model of the disease. While there is criticism of the bleomycin model, the stimulus of inflammation and accumulation of ECM in the lung mimics the acute immune responses and resulting hypertrophy of IPF in humans. Due to hsPUFIN1's location on chr21, the bleomycin model in the TcMAC21 mouse were used to study pulmonary fibrosis. The adaptation of the TcMAC21 line to the field of IPF was an alternative application, and due to how the TcMAC21 line was bred, the B6D2F1 was able to be used as a control group in this study.

A transchromosomic chr21 mouse model (TcMAC21) was bred from the transgenic B6D2F1 mouse line (Supplier: Trans Chromosomics. Breeder: BLEA Japan, Inc). Mice at 9 weeks of age were weighted and monitored for clinical signs and appearances.

Five groups of mice were used in the in vivo experiment: 1) B6D2F1 PBS (n=4), 2) B6D2F1 1 U/kg bleomycin (n=6), 3) TcMAC21 PBS (n=4), 4) TcMAC21 1 U/kg bleomycin (n=8), and 5) TcMAC21 1 U/kg bleomycin+100 μg ASO1 treatment (n=8). PBS was used as vehicle. Bleomycin was administered at day 0. PUFIN1 ASO1 (SEQ ID NO: 36) was intratracheally administered during the inflammation phase post-bleomycin, and the biological impact of the ASO treatment was assessed during the fibrotic phase. ASO treatment was performed on day 7 and day 14 post-bleomycin. Organs were harvested on day 21.

### Results:

### ASO Knockdown of PUFIN1 Attenuated Myofibroblast Gene Expression

TABLE 25
Sequences of ASOs targeting PUFIN1.

| | SEQ | | |
|---|---|---|---|
| Figure | ID | Sequence | |
| Legends | NO: | (from 5′ to 3′) | Modification |
| Scrl/SCRL | 2567 | AACACGTCTATACGC | LNA |
| (scramble) | | | |
| ASO1/ASO-1 | 36 | TCAGATAGAGTTAGAC | LNA |
| ASO2/ASO-2 | 37 | CTCAGTCTGGTCGTGC | LNA |

Notes:
The ASO1 and ASO2 sequences described in this table comprise 3 LNA modified nucleotides at the 5′ end and 2 LNA modified nucleotides at the 3′ end, forming 3-11-2 configuration of LNA-ASO GapmeR. Phosphorothioate linkages were incorporated between each of the nucleotides. Scramble ASO contained 3 LNA modified nucleotides at the 5′ end and 2 LNA modified nucleotides at the 3′ end resulting in 3-10-2. configuration of INA-ASO GanmeR

FIG. 16F shows that PUFIN1 expression was upregulated upon TGF-β treatment. FIG. 16G shows that PUFIN1 expression was elevated in IPF patients based on validation analysis using single-cell RNA-seq data reported by Adams et al., Sci Adv (2020), similar to results shown in FIG. 4A based on analysis using bulk RNA-seq data reported by Schafer et al., Nat Commun. (2017) and Sivakumar et al. ERJ Open Research (2019).

Myofibroblasts increase in expression of cytokines (e.g., TGFβ, CTGF, IL-6), increase in expression of contractile fibers (e.g., αSMA), increase in expression of extracellular matrix enzymes (e.g., MMP1, MMP3), and increase in expression of extracellular matrix components (e.g., COL1A1, COL3A1, FN) when affected by the fibrosis or undergoing fibrosis. In vitro fibroblast differentiation upon TGF-β treatment to induce fibrosis resulted in activated myofibroblasts that expressed higher levels of canonical profibrotic marker genes (data not shown).

As shown in FIGS. 6B and 6D-6G, PUFIN1 was knocked down when targeted by LNA ASOs (qPCR and bulk RNAseq). PUFIN1 expression level was decreased 80-90% when myofibroblasts were treated with LNA ASOs (ASO-1 (SEQ ID NO:36) and ASO-2 (SEQ ID NO: 37)) relative to the sample treated with scramble ASO. Expression levels of canonical markers of fibrosis (ACTA2, FN1, POSTN, FAP, COL1A1, and COL3A1) were also downregulated when myofibroblasts were treated with ASO-1 or ASO-2. As shown in FIG. 6F, ASO-1 treatment in pHLF decreases PUFIN1 and IPF gene signature expression. FB was untreated control. Similarly, most genes in target engagement panel showed similar expression trends as PUFIN1 and IPF gene signature upon ASO-1 treatment, as shown in FIG. 6G. FIG. 6C shows siRNA treatment reduced expression of PUFIN1 but not markers for fibrosis (POSTN, FAP, and COL3A1. Figure legends in FIG. 6B and FIG. 6G: * indicates p<0.05; ** indicates p<0.01; *** indicates p<0.001; * ** indicates p<0.0001; n=4 pHLF biological replicates; all comparisons are scramble vs ASO. Sequences of siRNA used in FIG. 6C were siRNA-1: 5′-CACCGGCAGUUAUUCCAAUtt-3′; and siRNA-2: 5′-GGCUCAGACAGAGAACUCAtt-3′. Neg (or control) was obtained from ThermoFisher/Invitrogen, Silencer™ Select Negative Control No. 1 siRNA (Catalog No. 4390843).

### PUFIN1 and Downstream Genes Shared TAD Regulatory Elements

As shown in FIG. 6H, PUFIN1 shared a topologically associating domain (TAD) with the genes downstream of it but not with genes upstream. This result was supported by analysis of bulk RNA-seq data from human IPF lungs reported by Schafer et al., Nat Commun. (2017), Nance et al., PLOS One (2014), and Luzina et al., Cell Immunol. (2018) (data not shown). Similar results that were reanalyzed from published data reported by Habermann et al., and Adams et al. (data not shown). Results from in vitro model using primary human lung fibroblasts (pHLF) correlated with results from in vivo analysis, supporting that PUFIN1 shares a topologically associating domain (TAD) with the genes located downstream of PUFIN1 When PUFIN1 expression was knocked down with treatment with ASO-1, 543 genes were significantly downregulated, and 173 genes were upregulated compared to the sample treated with scramble control. The top GSEA pathways that were downregulated were cilium chromosome, assembly, and fission pathways; defense response symbiont virus; establishment cell; cellular type interferon pathways; and mRNA trans-splicing addition; the top GSEA upregulated pathways were grouped into positive regulation and protein localization; aerobic ATP energy; nucleoside nucleotide and ribonucleotide ribose; ncRNA rRNA complex biogenesis; and RNA mRNA splicing (data not shown). Two of these downregulated genes (MX1 and MX2) were genes that are in the same regulatory TAD as PUFIN1. Also, three additional genes on chr21 (OAS1, OAS2, and OAS3) were also downregulated when PUFIN1 was knocked down (data not shown).

PUFIN1 was Associated with Cilia Regulatory Genes and Knockdown of PUFIN1 Downregulated these Genes

Weighted gene co-expression network analysis (WGCNA) classifies genes into modules whose member genes have highly correlated transcription. This can be used to predict the functional role and reprogramming potentials of lncRNAs, e.g., PUFINs. PUFIN1 co-expressed in a cilia and motility network, and the top genes PUFIN1 co-expressed with were dominated by genes with cilia and motility function (B9D1, LRRC23, SPACA9, TMEM231, CCDC65, ARHGAP39, CFAP300, and RAB36). The top pathways in this network were cilium organization, cilium assembly, microtubule-based movement, cilium-dependent cell motility, axoneme assembly, and extracellular transport (data not shown). The effect of ASO1 knockdown on PUFIN1 also significantly downregulated 137 genes in network (5% of total genes) and showed significant downregulation when compared to other networks (p=0.000241). This result suggests that PUFIN1 downregulated genes are preferentially expressed in the PUFIN1 module. Further, rs188240612 variant and risk allele was identified and located in CFAP300 gene (data not shown).

An analysis using scRNAseq in vivo IPF data provided that the genes associated with PUFIN1 were predominately associated with epithelial cilia/upper airway epithelial cells (data not shown). PUFIN1's discovery in vitro showed that it was a lncRNA that was significantly involved in myofibroblast regulation, and its association with cilia genes suggested that PUFIN1 was associated with epithelial-to-mesenchymal transition (EMT) in ciliated cells.

### PUFIN1 Downregulation in a Humanized Mouse Model

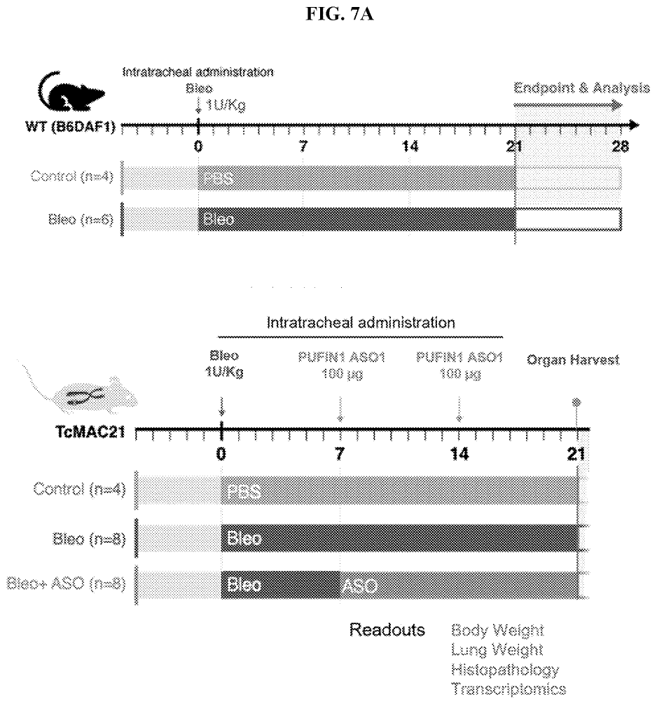

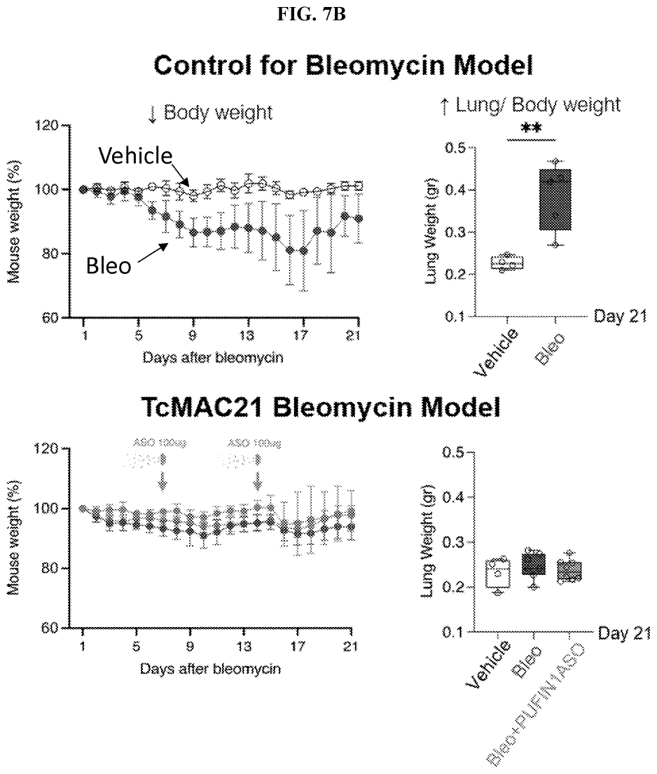

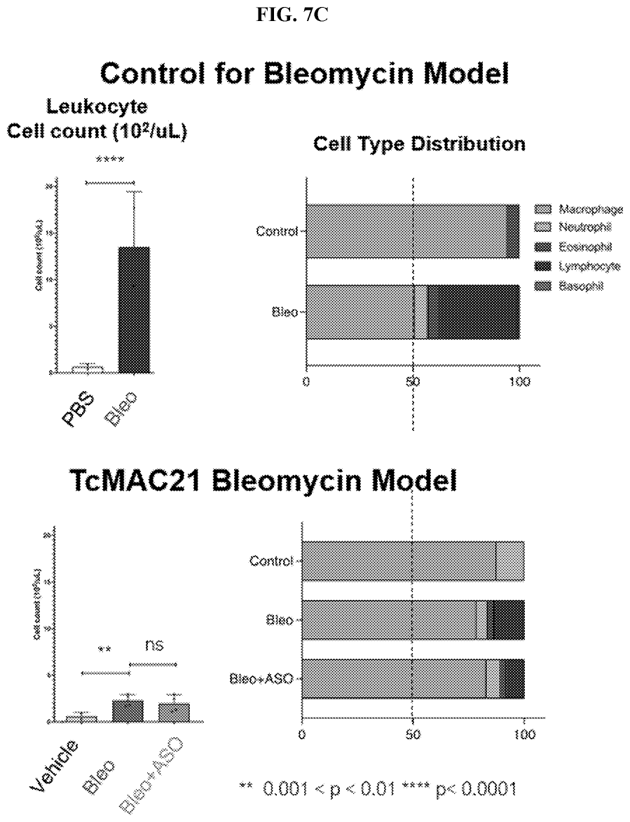

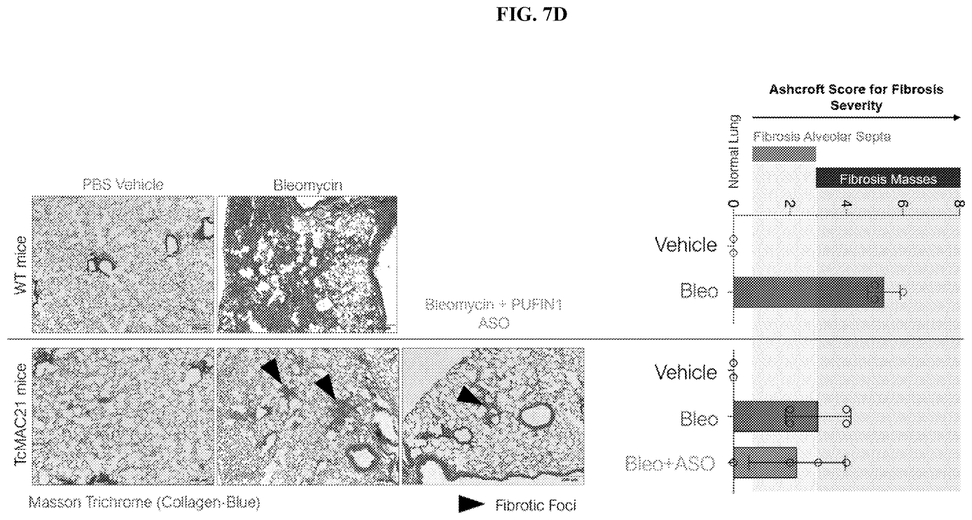

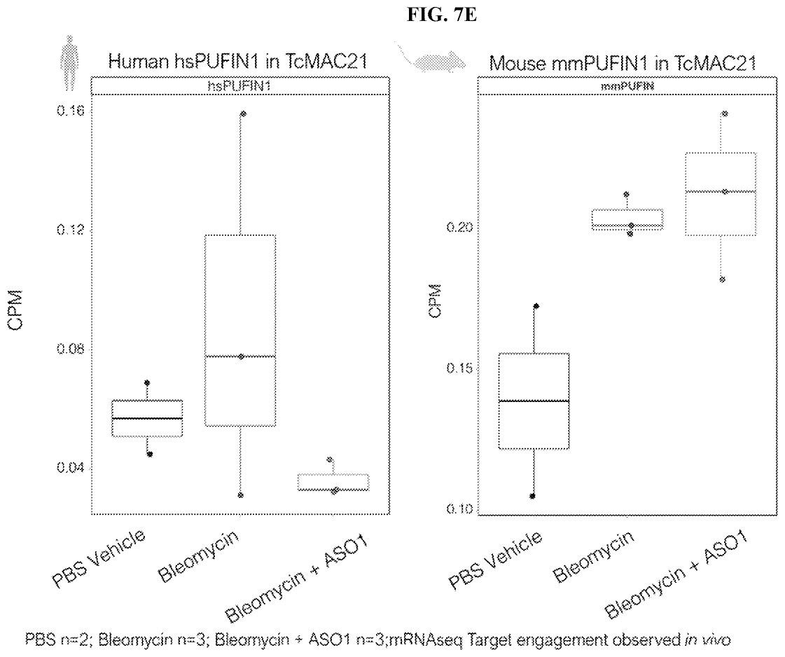

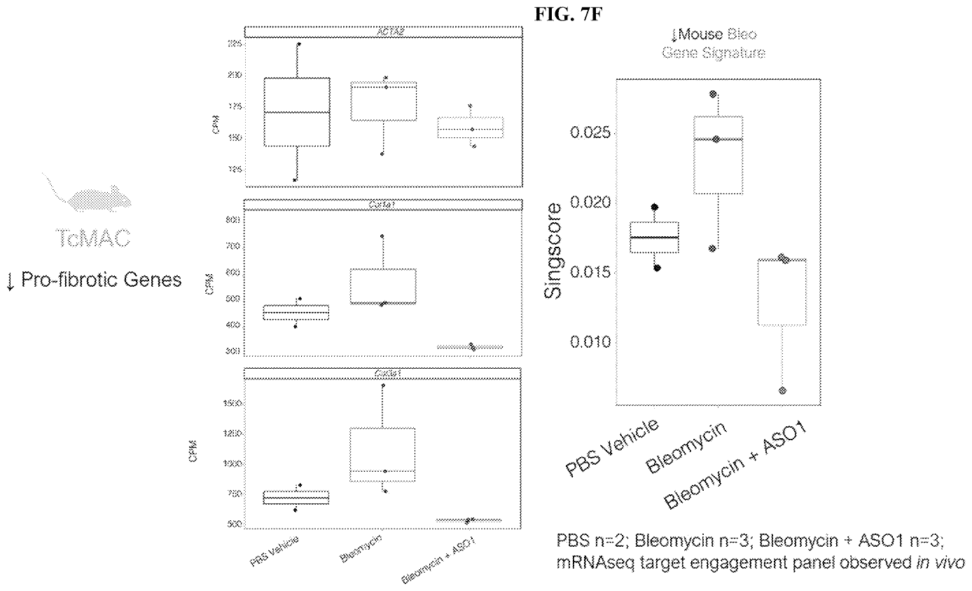

As shown in FIG. 7A, a mouse model was generated to translate ASO-1 treatment in vitro into an in vivo. As for control of bleomycin model, WT mice (B6DAF1) were used (FIG. 7A top panel). As shown in FIG. 7A bottom panel, in TcMAC21 Bleomycin Model, mice were administered with ASO via intratracheal administration on day 7 and 14 post-bleomycin treatment (during inflammation phase). Organs were harvested on day 21 (during fibrotic phase) for further analysis. A transchromosomic mouse model with an entire human chr21 inserted was generated (diagram not shown). Sequence of ASO used in a mouse model to target PUFIN1 and modification were shown in Table 26.

TABLE 26
Sequence of ASO used in a mouse model

| | SEQ | | |
|---|---|---|---|
| Figure | ID | Sequence | |
| Legends | NO: | (from 5′ to 3′) | Modification |
| ASO1 | 36 | TCAGATAGAGTTAGAC | LNA |

Notes:
the ASO1 sequence described in this table comprise 3 LNA modified nucleotides at the 5′ end and 2 LNA modified nucleotides at the 3′ end, forming 3-11-2 configuration of LNA-ASO GapmeR. Phosphorothioate linkages were incorporated between each of the nucleotides. PBS was used as a negative control.

FIG. 7B shows that the TcMAC21 bleomycin mice treated with ASO showed steady weight comparable to control animals. As shown in FIG. 7C, bleomycin treatment in TcMAC21 mice resulted in immune activation, as indicated with increase in leukocyte counts, compared to vehicle treated mice. Upon ASO1 treatment, the bleomycin induced increase of leukocytes was abrogated, indicating that suppression or knockdown of PUFIN1 expression using ASO could reverse or prevent the immune activation upon onset or development of fibrosis.

When treated with bleomycin, the mouse tissue developed fibroblast foci, which is shown in FIG. 7D. When bleomycin treated animals were treated with ASO to knockdown PUFIN1, histology of the mouse tissue showed significant attenuation of fibrotic foci, measured by Ashcroft score, indicating that suppression or knockdown of PUFIN1 expression using ASO could reverse or prevent the immune activation upon onset or development of fibrosis.

FIG. 16D shows picrosirius red staining of samples from TcMAC21 mice model. Consistently with results in FIG. 7D, collagen content deposited in the pulmonary interstitium was decreased after PUFIN1 ASO1 treatment, indicating that suppression or knockdown of PUFIN1 expression using ASO could reduce the development of fibrosis.

In IPF patient's lung, eosinophils can infiltrate lung tissue, and these immune cells can induce fibroblast-to-myofibroblast differentiation. Increased eosinophil abundance is one of a risk factor for acute exacerbation and negatively correlates with survival. FIG. 16E shows H&E staining, and PUFIN1 ASO1 treatment abrogated bleomycin-induced eosinophil infiltration in transgenic humanized mouse lungs, indicating that suppression or knockdown of PUFIN1 expression using ASO may increase the survival rate of the subject affected by pulmonary fibrosis (e.g., IPF).

As shown in FIGS. 7E-7F, RNA samples were obtained from the biopsied lung tissue from the mouse model as described in FIG. 7A, and the RNA samples were sequenced. The RNA sequencing data showed that the expression of hsPUFIN1 was knocked down in bleomycin/ASO treated animals compared to bleomycin treated animals (FIG. 7E). Col1a1 and Col3a1 were also downregulated in bleomycin/ASO treated animals compared to the animals treated with bleomycin only (FIG. 7F left panel). Consistently, the IPF gene score (Singscore) was decreased after ASO1 treatment in bleomycin (FIG. 7F right panel).

Multiple gene pathways were affected by the modulation of PUFIN1 expression. GSEA pathway analysis in TcMAC21 Bleomycin+ASO treated mice showed that most upregulated pathways were actomyosin ameboidal-type regulation catabolic; autophagy utilizing and autophagic mechanisms; striated tissue development and differentiation; response to peptide hormone stimulus; and internal peptidyl-lysine and acetylation modification. GSEA pathway analysis showed cilia dependent pathways were the top downregulated pathways in ASO treated mice. Other pathways that were downregulated in TcMAC21 Bleomycin+ASO treated mice were dynein extracellular arm fluid; cilium-dependent flagellated sperm motility; negative division activation factor; MHC class II protein; and regulation interleukin-1 beta production. As shown in FIG. 7G, genes associated with PUFIN1 co-expression module comprised, for examples, B9D1, IQCG, LRRC23, TMEM231, SPACA9, CCDC65, ARHGAP39, C9orf116, CFAP300, and RAB36.

The modulation of cilia expressing pathways and genes upon knockdown of PUFIN1 by ASO1 indicated that PUFIN1 is closely tied to cilia expressing pathways and genes. Also, analysis by integration of scRNAseq databases supported that PUFIN1 showed preferential expression in upper airway epithelial cells. The analysis also showed that expression of PUFIN1 was linked to expression of several genes (LRRC23, CFAP300, CCDC65) that have human genetic mutations associated with lung function (CFAP300), cilia dyskinesia (LRRC23), and lung adenocarcinoma (CCDC65). Epithelial to mesenchymal transition (EMT) in ciliated cells are often observed in IPF, and the loss of cilia in cells expediates the fibroblast cell state transition to the myofibroblast state. As such, PUFIN1's association with cilia gene expression and ciliated pathways suggests that PUFIN1 can be a regulator of EMT in ciliated airway cells. Also, the association of these genes with IPF pathology further supports the strong association with PUFIN1 expression and IPF pathology.

PUFIN1 shares a TAD domain with nearby genes at chromosome 21. Because of PUFIN1's location on chr21, a humanized mouse model that contained an additional chromosome was utilized to study knockdown of human lncRNA (hsPUFIN1) in vivo. The utility of this unique in vivo humanized mouse model showed promise for preclinical testing of the therapeutic potential of PUFIN1 ASO knockdown. The observed attenuation of fibrotic foci (histology) in these mice following ASO1 treatment decreased the expression of fibroblast foci (FIG. 7D), which are distinct to human IPF pathophysiology. These fibrotic foci in mice treated with bleomycin ASO1 are lessened (by count) compared to foci in mice treated with only bleomycin (FIG. 7D).

In the TcMAC21 humanized mouse model, when comparing bleomycin and bleomycin/ASO1 treated mice, GSEA analysis (FIG. 7F) showed a similar pathway analysis that was dominated by cilia and motility gene express pathways, which confirmed the cilia driven gene signature that was derived in vitro.

Myofibroblast state is associated with increased ECM deposition, increased interleukin secretion, and increased detection of HAS1+ and PLIN2+myofibroblast populations (Sieber, P. et al. JCI Insight (2023)). HAS1+ and PLIN2+fibroblasts are pathogenic cell population in IPF and are detectable before the complete transition into myofibroblasts. Further analysis and validation using scRNAseq data published by Habermann, A. et al. Sci Adv. (2020) showed that PUFIN1 expression was upregulated in myofibroblasts of IPF patients (data not shown). PUFIN1 expression was also detectable in HAS1+fibroblasts. In addition, PUFIN1 expression was detected during the transition from epithelial cells to myofibroblasts in IPF patients (based on analysis using public data from Adams et al., 2020). Further, PUFIN1 expression increased in myofibroblasts in the injured lung, and was depleted from epithelial (cilia) cell types with lung damage.

### Super-Enhancer Analysis and Results

Super-enhancer refers to an enhancer that differs from typical enhancers in terms of size, transcription factor density, ability to activate transcription, and sensitivity to perturbation. Super-enhancer can be larger, bind multiple transcription factors or have greater density of potential transcription factor binding domains, have elevated ability to activate transcription, and are more sensitive to perturbation. The Rank Ordering of Super-Enhancers (ROSE) algorithm was utilized to identified super-enhancers in lung fibroblasts and myofibroblasts, and these super-enhancers are referred to as lung super-enhancer.

PROseq, measuring nascent transcription, was used to identify the PUFIN1 promoter. The ROSE algorithm was performed, and a super-enhancer (in the region where PUFIN1 is transcribed from) was identified. PUFIN1 was identified as a lncRNA transcript that was transcribed from a lung super-enhancer, with a RUNX1 motif located within PUFIN1 promoter (FIG. 16A). RUNX1 is a lung-relevant transcription factor. These results suggested that PUFIN1 was located in transcriptionally active area that can be opened under conditions of cell stress which can lead to IPF. Further, PUFIN1 ASO1 (SEQ ID NO: 36) treatment led to the depletion of PUFIN1 transcript.

PUFIN1 epigenomic activity was investigated by quantification of histone 3 lysine 27 acetylation (H3K27ac) signal. As shown in FIG. 16B, epigenomic profiling revealed that PUFIN1 was not only lung specific but resided within a super-enhancer. PUFIN1 expression in human lung fibroblasts (HLFs) correlated with super-enhancer marked by H3K27Ac and was upregulated in the disease state. Abbreviation in FIG. 16B: NHLF, normal healthy lung fibroblasts; NHDF, normal healthy dermal fibroblasts.

### Target Engagement Panel (TEP) Study of ASO Targeting PUFIN1

In vitro dosage response experiment using PUFIN1 ASO1 (SEQ ID NO: 36) was performed to define PUFIN1 Target Engagement Panel (TEP).

### Methods:

### Dosage Dependent Experiment

In vitro cell culture methods and experiments were performed as described in previous examples. The effect of ASO-1 (SEQ ID NO: 36) to modulate various downstream gene expression was examined at concentrations of 0.5 nM, 1 nM, 5 nM, 10 nM, and 20 nM. Results from this experiment were used to cluster RNA transcripts showing dose-dependent expression where the lncRNA (PUFIN1) was targeted by ASO1 (SEQ ID NO: 36). RNA extraction, qPCR, and bulk RNAseq were performed as described in the previous examples. Expression of these dose-dependent genes were clustered to form a Target Engagement Panel (TEP).

### Target Engagement Panel (TEP)

For PUFIN1 Target Engagement Panel (TEP) construction, an unbiased hierarchical agglomerative clustering of all genes based on their expression at 5 different concentrations of PUFIN1 ASO1, measured by RNA-seq, was performed. Dose responsive genes identified from ASO dose response and bulk RNA-seq were analyzed. Genes in clusters that displayed a significant dose-dependent downregulation were considered as candidate PUFIN1 TEP genes.

TEP construction involved 2 clustering steps, the first pass clustering which identified genes responsive to ASO treatment, and the second clustering, where the most responsive genes, e.g., in clusters 6 and 7, in this experiment, were further reclustered to define the subcluster(s) with the strongest response to the ASO. Briefly, an initial Ward hierarchical clustering was performed to establish the first pass clustering to identified subsets of genes that response to ASO treatment in a dose-dependent manner. Genes that were best performing from the initial clusters were further analyzed (the second clustering) using similar methodology, thereby narrowing down the subsets of genes that showed the strongest dose-dependent manner to the ASO treatment. Genes from the most responsive clusters from this second clustering were considered in TEP.

### Results:

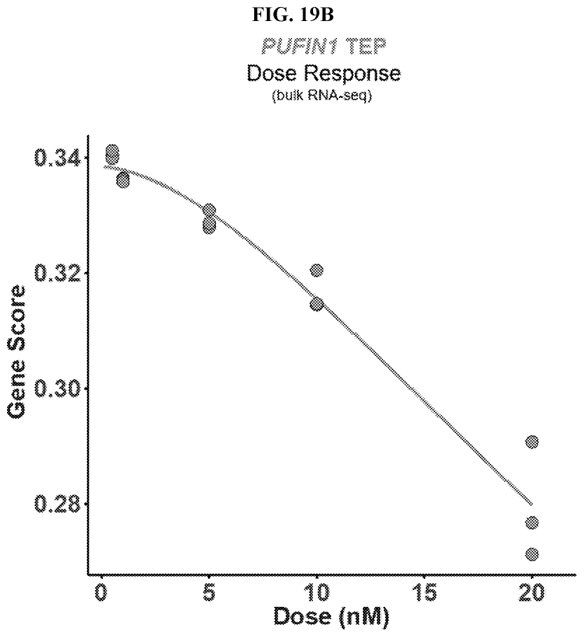

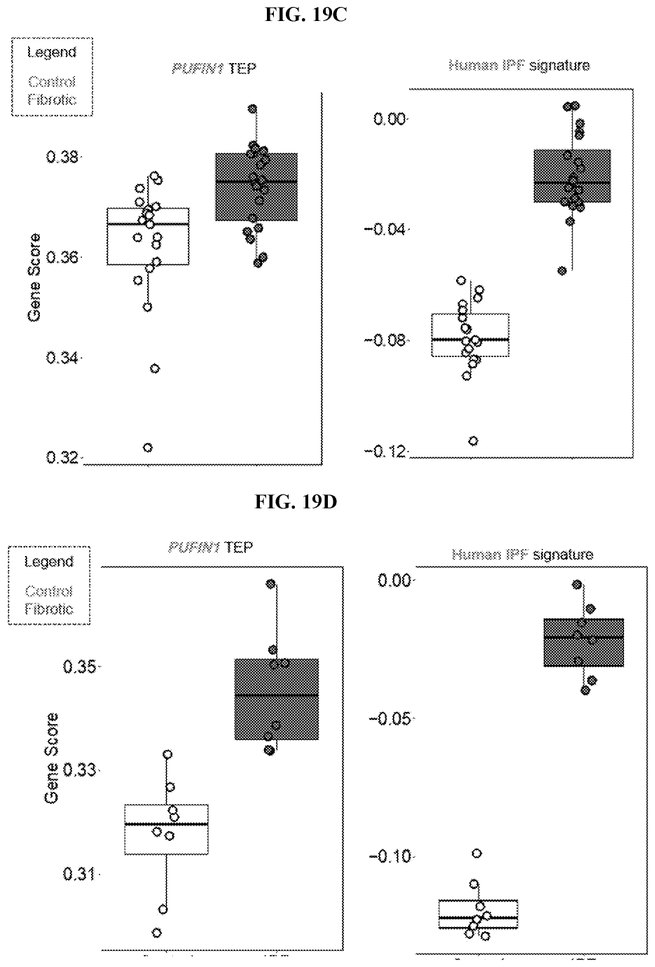

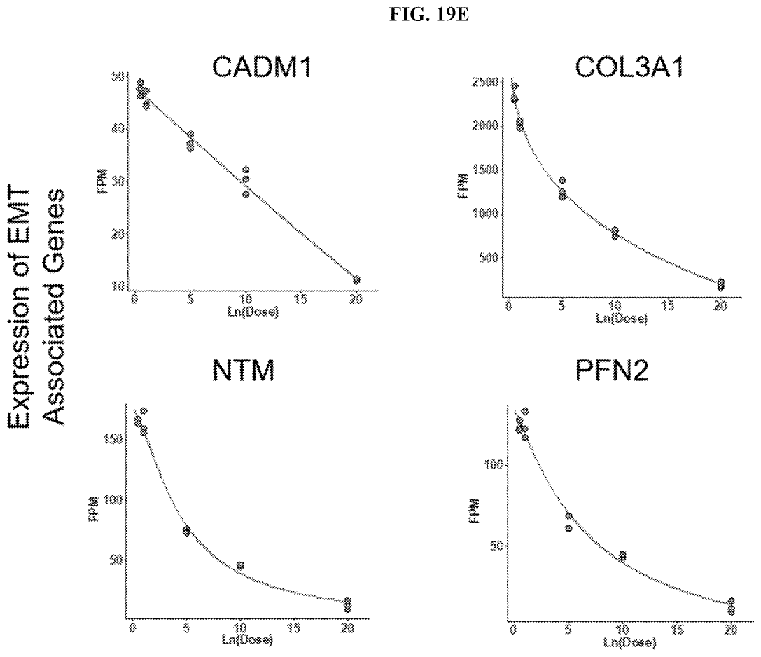

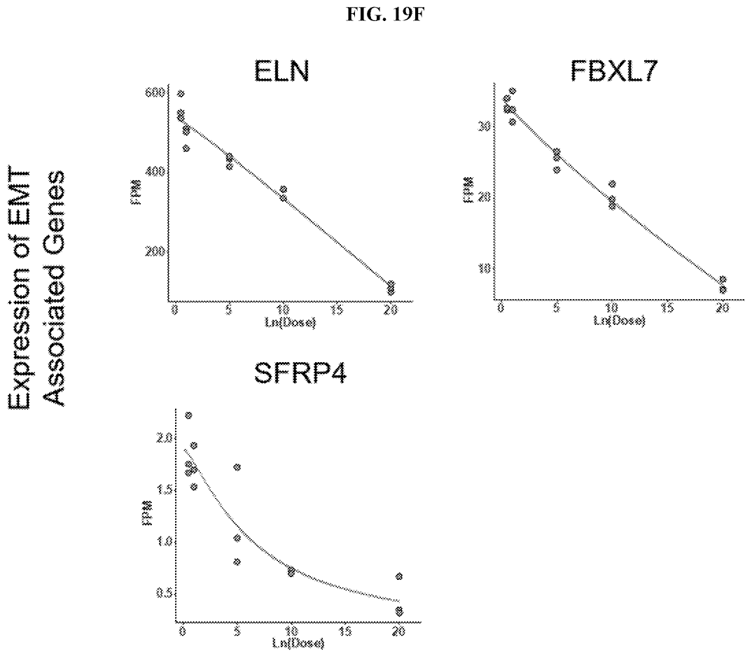

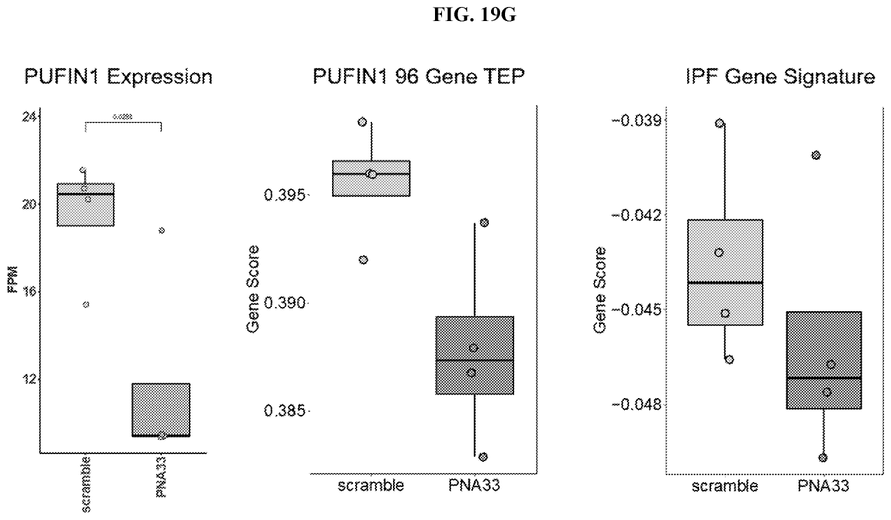

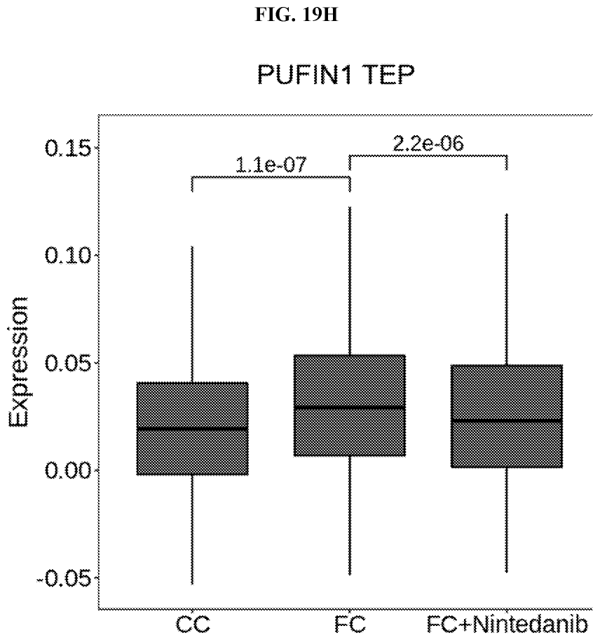

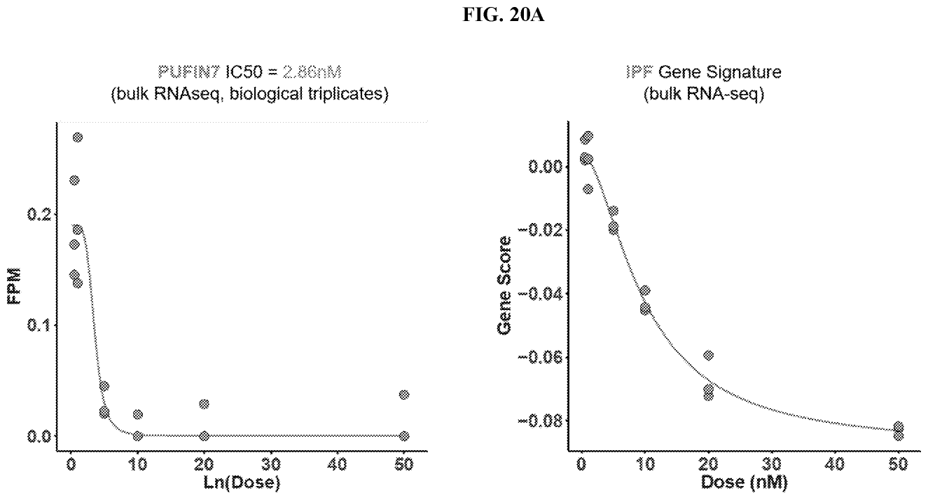

FIG. 19A shows that PUFIN1 expression was dose-dependent upon ASO-1 (SEQ ID NO: 36) treatment. Also, IPF gene signature (the genes that go up in IPF condition from Table 18) was downregulated in a dose-dependent manner upon ASO-1 treatment. The first pass clustering resulted in 7 initial gene clusters. Clusters 6 and 7 from this step were further reclustered for TEP refinement. The second clustering step resulted in final 5 gene clusters. The most responsive gene clusters of the second clustering step, which were clusters 3 and 4, were identified as PUFIN1 TEP, which comprises 96 genes. List of genes in PUFIN1 TEP are shown in Table 31. FIG. 19B shows dose response curve calculated from 96 dose-responsive TEP genes upon ASO1 (SEQ ID NO: 36) treatment.

TABLE 31
Genes in the PUFIN1 TEP upon ASO1 treatment.

| | | | | | | | | | |
|---|---|---|---|---|---|---|---|---|---|
| DOK6 | IMMP2L | SYT16 | TIA1 | KLF8 | PLN | TNIK | HDGFL3 | SDK1 | OPCML |
| ABHD3 | NAALADL2 | COL15A1 | LSAMP | FBXL7 | RNF150 | CILP | ZC3H12B | ELN | ROBO2 |
| MYCBP2 | CHSY3 | NTM | SLC8A1 | RUNX2 | ZHX2 | GPC4 | PLCB1 | PARPBP | CADM1 |
| ZDHHC16 | MAPK10 | PITPNA | PARD3B | LINC01239 | WDR70 | P3H2 | ABCC1 | PTPRE | ICA1L |
| TPT1-AS1 | ABTB3 | EYA4 | COL3A1 | SFRP4 | TENM4 | SNCAIP | PRG4 | LINC02593 | BCYRN1 |
| ATAD2B | C1QTNF7 | SORBS2 | NOVA1 | SPON1 | PRICKLE2 | CAMK1D | CHRM2 | NEDD9 | SH3YL1 |
| GPC6 | TDRP | PFN2 | MNAT1 | PCED1B | FARS2 | BRINP3 | SLC10A7 | TXLNB | |
| CMSS1 | FRMPD4 | IGF1 | ITGA11 | BBS9 | CDHR3 | KIF26B | MDFI | OLFM2 | |
| PRKG1 | MYOCD | CACNA1C | PLXDC2 | DERL3 | NEGR1 | PAPPA2 | XIST | CAPN1 | |
| CCBE1 | FBXL17 | LINC01614 | TYRP1 | PTPRG | MMP11 | ORC3 | BACH2 | PAMR1 | |

Expression of gene sets of PUFIN1 TEP was validated using the data available in public bulk RNA-seq database. FIGS. 19C and 19D show that expression of gene sets of PUFIN1 TEP is correlated with human IPF signature (genes listed in Table 18) based on analysis of RNA-seq data reported by Schafer et al., Nat Commun. (2017) (FIG. 19C) and Luzina et al., Cell Immunol. (2018) (FIG. 19D). These results suggested that genes of PUFIN1 TEP panel were highly expressed in IPF patients and that targeting PUFIN1 provided potential approach to modulate disease or revert disease prognosis. Genes in PUFIN1 TEP that were most sensitive to PUFIN1 ASO1 treatment comprised genes associated with collagen-containing extracellular matrix (SPON1, COL3A1, CILP, ELN, and PRG4), genes associated with basement membrane (COL15A1 and P3H2), and genes associated with cell-cell adhesion (ROBO2, SDK1, TENM4, CADM1, CDHR3, and GPC4).

Results of bulk RNA-seq heatmap of the top 100 most downregulated genes based on PUFIN1 ASO1 dose response showed that these downregulated genes displayed dose-responsive decrease associated with PUFIN1 ASO treatment (data not shown). Protein-protein interaction network was reconstructed, and results revealed key genes in fibrosis that were downregulated in PUFIN1 ASO treatment. PUFIN1 TEP protein-protein functional interaction network was generated by STRING Database analysis, and genes that were shown to interact with each other were shown in Table 36.

TABLE 36
Genes in PUFIN1 TEP that were predicted to have protein-protein functional interaction.

| | | | | | | | | |
|---|---|---|---|---|---|---|---|---|
| BBS9 | CHRM2 | GPC4 | MMP11 | ORC3 | SFRP4 | CDHR3 | CDH11 | CCBE1 |
| BGN | COL10A1 | HDGFL3 | MNAT1 | PAPPA2 | SLC10A7 | FBXL17 | EYA4 | ELN |
| BRINP3 | COL11A1 | HNRNPU | MYCBP2 | PLCB1 | SLC8A1 | MFAP5 | MDFI | LSAMP |
| CACNA1C | COL15A1 | IGF1 | MYOCD | PLN | SORBS2 | OPCML | OLFM2 | NTM |
| CADM1 | COL3A1 | IMMP2L | NEDD9 | PRKG1 | TDRP | SDK1 | RUNX2 | ROBO2 |
| CAMK1D | COL5A2 | ITGA11 | NEGR1 | PTPRD | TENM4 | ZHX2 | ZDHHC16 | TYRP1 |
| CAPN1 | CSMD1 | KIF26B | NOVA1 | PTPRG | TNIK | | | |

Transcription factor enrichment analysis in genes of PUFIN1 TEP was performed using ChIP-X Enrichment Analysis version 3 (ChEA3), and results showed that SMAD4, NFκB1, SMARCD1, WT1, PAX3-FKHR, KDM2B, SUZ12, TCF4, SUZ12, and TALI, were among the top transcription factors that regulated the 96 genes in PUFIN1 TEP. ENRICHR analysis of top Reactome Gene Ontology Pathways associated with genes in PUFIN1 TEP was performed, and results show that top pathways were post-translational modification: synthesis of GPI-anchored proteins; defective EXI1 causes exostoses 1, TRPS2, and CHDS; chondroitin sulfate/dermatan sulfate metabolism; extracellular matrix organization; defective B3GALT6 causes EDSP2 and SEMDJL1; defective B2GAT3 causes JDSSDHD; defective B4GALT7 causes EDS, progeroid type; collagen biosynthesis and modifying enzymes; HS-GAG degradation; and GAG synthesis requires tetrasaccharide linker sequence. ENRICHR analysis of top Wikipathways associated with genes in PUFIN1 TEP was also performed and results show that top pathways were Wnt signaling; miR509 P3 alteration of YAP1 ECM Axis; extracellular vehicles in the crosstalk of cardiac cells; miRNA targets in the ECM and membrane receptors; arrhythmogenic right ventricular cardiomyopathy; calcium regulation in cardiac cells; Alzheimer 39 S disease and miRNA effects; Alzheimer 39 S disease; GPR143 in melanocytes and retinal pigment epithelium cells; and integrin mediated cell adhesion.

Gene Ontology (GO) Pathway Analysis was performed based on GO Molecular Processes, GO Cellular Components, and GO Biological Processes, revealing ECM and collagens GO pathways. Top pathways in GO molecular processes comprised transmembrane receptor protein phosphatase activity; transmembrane receptor protein tyrosine phosphatase activity; phosphatidylinositol-4,5-bisphophate binding; protein tyrosine phosphatase activity; phosphatidylinositol biphosphate binding; phosphatidylinositol binding; and protease binding. Top pathways in GO cellular components comprised collagen-containing extracellular matrix; golgi lumen; basement membrane; intracellular organelle lumen; Hrd1p ubiquitin ligase ERAD-L complex; nuclear stress granule; Hrd1p ubiquitin ligase complex; SCF ubiquitin ligase complex; dendrite; and clathrin-coated endocytic vesicle membrane. Top pathways in GO biological processes comprised cell-cell adhesion via plasma-membrane adhesion molecules; regulation of smooth muscle cell differentiation; integrin-mediated signaling pathway; calcium ion transport into cytosol; insulin-like growth factor receptor signaling pathway; relaxation of cardiac muscle; relaxation of muscle; positive regulation of glycoprotein biosynthetic process; membrane depolarization during action potential; and regulation of synapse assembly. Based on analysis using human molecular signatures database (DB Sig or MSigDB), which result is shown in Table 37 below, epithelial Mesenchymal Transition (EMT) pathway was significantly associated with the genes from the PUFIN1 TEP. Key genes identified are Collagen type III alpha 1 chain (COL3A1), Neurotrimin (NTM), elastin (ELN), Profilin 2 (PFN2), Secreted frizzled related protein 4 (SFRP4), Cell adhesion molecule 1 (CADM1), and F-box and leucine rich repeat protein 7 (FBXL7), and as shown in FIGS. 19E and 19F, the expression of these genes was dose-responsive to PUFIN1 ASO treatment.

TABLE 37
Pathways associated with genes from the PUFIN1 TEP
based on human molecular signatures database.

| | | | Ad- | | Com- |
|---|---|---|---|---|---|
| In- | | | justed | Odds | bined |
| dex | Name | P-value | p-value | Ratio | score |
| 1 | Epithelial | 0.0004075 | 0.008966 | 6.77 | 52.87 |
| | mesenchymal | | | | |
| | transition | | | | |
| 2 | myogenesis | 0.07172 | 0.6097 | 3.23 | 8.5 |
| 3 | PI3K/AKT/ | 0.09065 | 0.6097 | 4.09 | 9.82 |
| | mTOR | | | | |
| | signaling | | | | |
| 4 | UV | 0.1521 | 0.6097 | 2.96 | 5.58 |
| | response Dn | | | | |
| 5 | angiogenesis | 0.1592 | 0.6097 | 5.98 | 10.98 |
| 6 | Fatty acid | 0.1758 | 0.6097 | 2.69 | 4.68 |
| | metabolism | | | | |
| 7 | reactive | 0.2103 | 0.6097 | 4.35 | 6.79 |
| | oxygen | | | | |
| | species | | | | |
| | pathway | | | | |
| 8 | mitotic | 0.2476 | 0.6097 | 2.13 | 2.97 |
| | spindle | | | | |
| 9 | KRAS | 0.2494 | 0.6097 | 2.12 | 2.94 |
| | signaling up | | | | |
| 10 | peroxisome | 0.3945 | 0.6198 | 2.02 | 1.88 |

Furthermore, based on analysis of public scRNAseq data (Habermann, A. et al., Sci Adv. (2020)), expression of genes in PUFIN1 TEP was observed in a dose-dependent manner in myofibroblast sample from IPF patient. Consistently, expression of genes in PUFIN1 TEP in myofibroblasts was shown to have similar trend as PUFIN1 expression (data not shown). As shown in FIG. 19G, which was analyzed from public bulk RNAseq data (Ahangari et al., JCI Insight. (2023)), PUFIN1 expression was predicted to decrease in an ex vivo human lung tissue model after post-fibrotic treatment, e.g., PNA33 (a peptide nucleic acid inhibitor of miR-33 for fibrotic treatment). Consistently, expression of genes in PUFIN1 TEP and expression of genes in IPF gene signature were also predicted to decrease in this dataset. This result indicated that targeting PUFIN1 can have anti-fibrotic effects.

Based on analysis from scRNAseq results of ex vivo model reported by Lang N et al., Sci Transl Med. (2023), genes in PUFIN1 TEP and in IPF gene signature showed highest expression in myofibroblasts. In this report, three ex vivo conditions were tested: control cocktail (CC), fibrotic cocktail (FC), and fibrotic cocktail+Nintedanib (FC+Nintedanib). FIG. 19H shows that expression of genes in PUFIN1 TEP was increased upon FC treatment and was decreased upon anti-fibrotic treatment (FC treatment along with Nintedanib, an anti-fibrotic drug), suggesting that genes in PUFIN1 TEP can be used to predict anti-fibrotic effects and/or efficacy of a drug for treating fibrosis.

In addition, expression of genes in PUFIN1 TEP was determined using mice data. FIG. 16C shows results of bulk RNA-seq of genes in IPF gene signature and genes in PUFIN1 TEP evaluated in mice model. This result shows that PUFIN1 ASO1 treatment decreased/reversed expression of genes in IPF gene signature as well as expression of genes in PUFIN1 TEP. Unbiased heatmap display of in vivo differentially expressed genes was generated and results showed that genes whose expression were induced by bleomycin were attenuated upon hPUFIN1 ASO treatment (data not shown). Examples of genes relevant to IPF were collagen-containing extracellular matrix (e.g., GDF15, MGP, LOXL1), extracellular matrix organization (e.g., COL28A1, ELN), and cellular response to chemical stress (e.g., GPX7, PYCR1). These data confirmed in vivo proof-of-concept for hPUFIN1 ASO1 specificity and reversion of disease transcription.

Overall, PUFIN1 TEP can be used to investigate PUFIN1 mechanism of action (MoA) or to access or compare ASO potency and efficacy.

Further, identification of PUFIN1 as a target for IPF treatment provides opportunity and advantages for IPF therapeutic development. Data from in vitro, in vivo, and ex vivo models, demonstrated an anti-fibrotic effect in lungs upon PUFIN1 ASO treatment, and utilizing these models allowed translational assessment of PUFIN interventions as well. Overall, these results showed that ASOs targeting PUFIN1 were highly effective, and upon ASO knockdown of PUFIN1, a significant reduction in myofibroblast gene expression was observed. These results provided evidence of the potential utility of ASO knockdown of PUFIN1 as a therapeutic strategy. Further, these results provided important insights into the cellular and molecular mechanisms underlying IPF and highlight the potential of using ASOs targeting PUFIN1 as a therapeutic strategy to treat IPF.

### Example 3: ASO Knockdown of PUFIN7 Attenuated Myofibroblast Gene Expression

In this example, ASO sequences were developed in silico to target PUFIN7, as identified in the Example 1.

Methods of ASO design and generation as well as experiments were performed as described in the Example 2.

Antisense oligonucleotides (ASOs) comprising locked nucleic acid (LNA) modifications (LNA ASOs) were designed and generated. Table 7 shows the ASO sequences generated in silico to target PUFIN7. ASO sequences targeting PUFIN7 that were selected for further testing can be found in Table 4 or Table 27.

### Results:

ASO knockdown of PUFIN7 attenuated myofibroblast gene expression

Table 27 shows modified ASO sequences targeting PUFIN7 with LNA.

TABLE 27
Sequence of ASOs targeting PUFIN7.

| | SEQ | | |
|---|---|---|---|
| Figure | ID | Sequence | Modifi- |
| Legends | NO: | (from 5′ to 3′) | cation |
| Scr/Scramble | 2567 | AACACGTCTATACGC | LNA |
| L1 | 27 | AGCATAACTCAGCCGT | LNA |
| L2 | 28 | TCTTACGTGTCTGAAG | LNA |
| L5 | 31 | GACCCTAGCACTTGAG | LNA |
| L6 (ASO L6) | 32 | TGAAGTAGCCATAGCC | LNA |
| L7 | 33 | GAAGATTACCTAGCAG | LNA |
| L8 | 34 | GCAGTACCAGAACTAT | LNA |
| L9 (ASO L9) | 35 | GTCACCACATGTTAGT | LNA |

Notes:
The L1, L2, L5, L6, L7, L8, and L9 sequences described in this table comprise 3 LNA modified nucleotides at the 5′ end and 2 LNA modified nucleotides at the 3′ end, forming 3-11-2 configuration of LNA-ASO GapmeR. Phosphorothioate linkages were incorporated between each of the nucleotides. Scramble ASO comprises 3 LNA modified nucleotides at the 5′ end and 2 LNA modified nucleotides at the 3′ end, forming 3-10-2 configuration of LNA-ASO GapmeR.

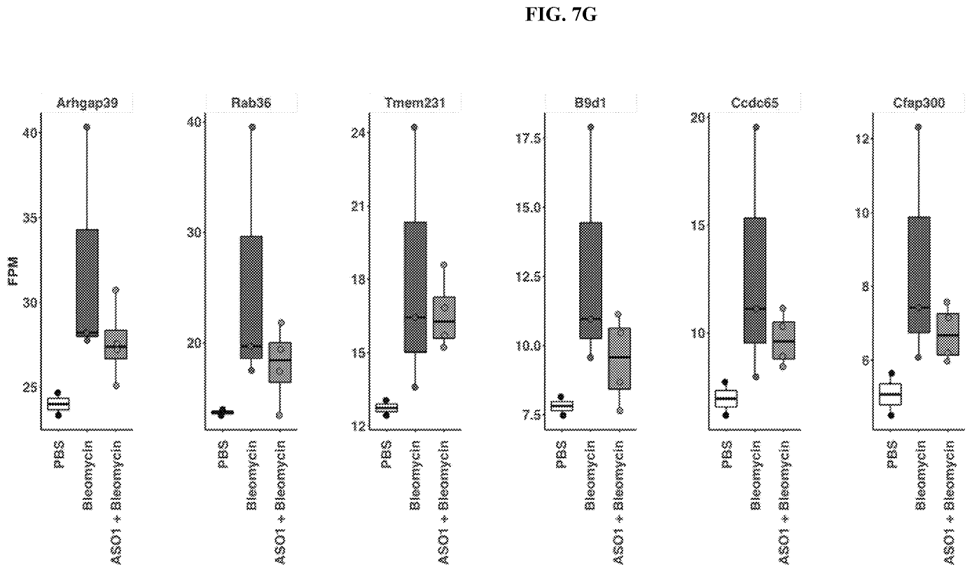

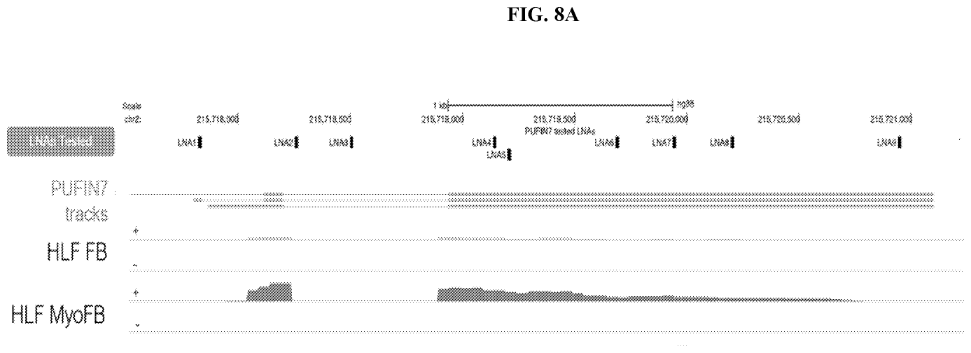

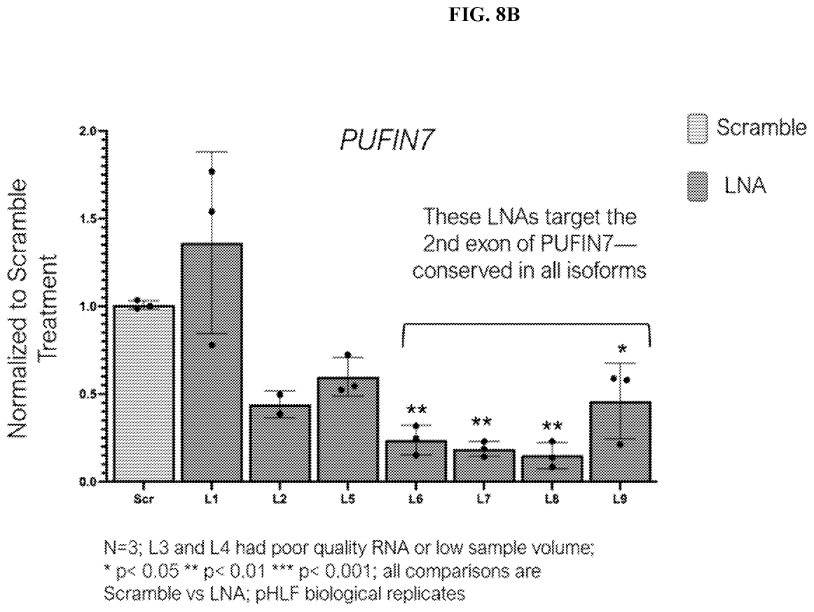

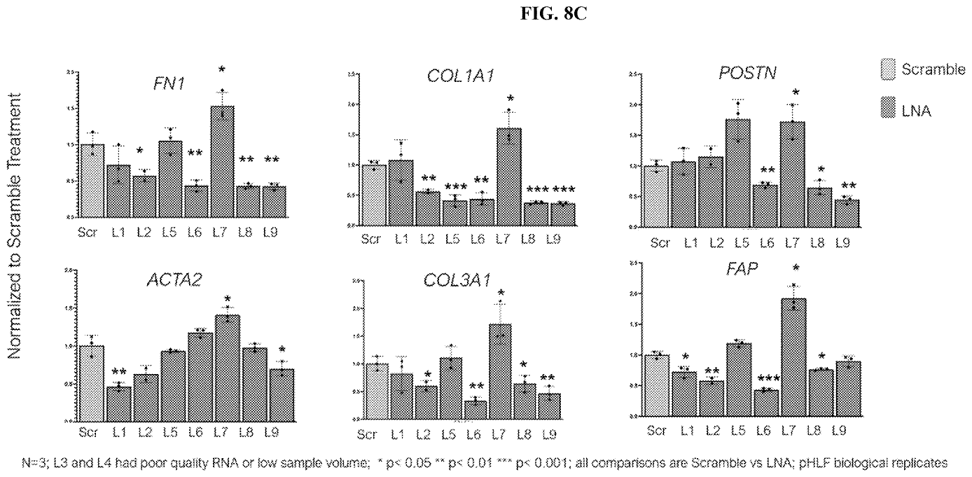

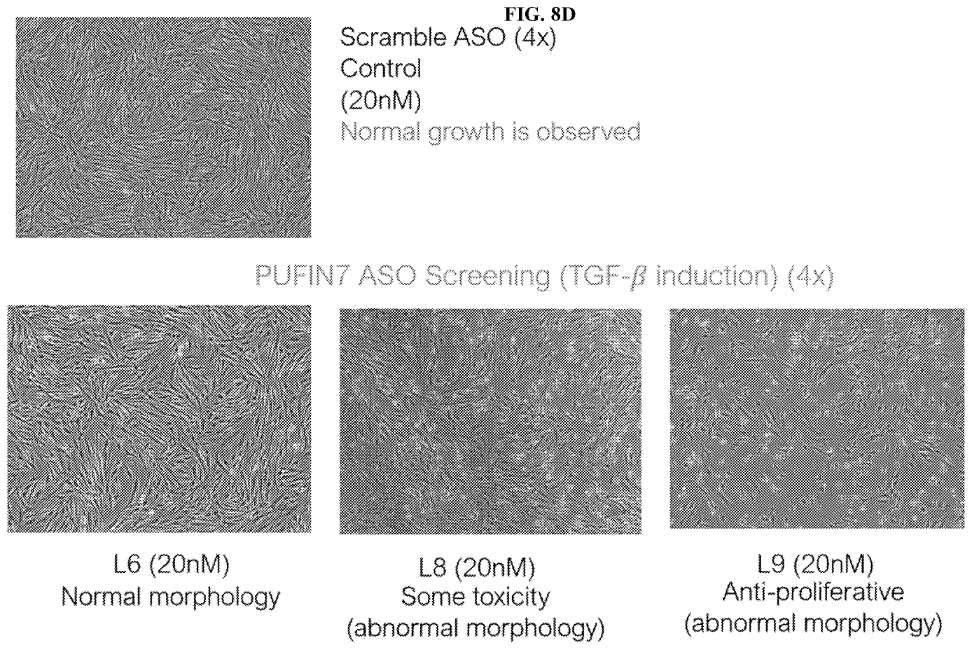

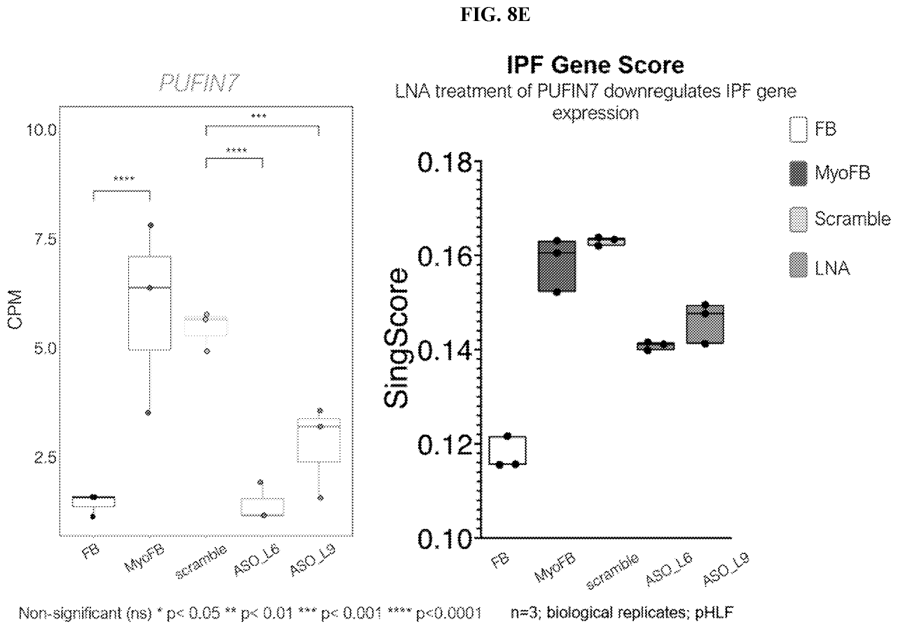

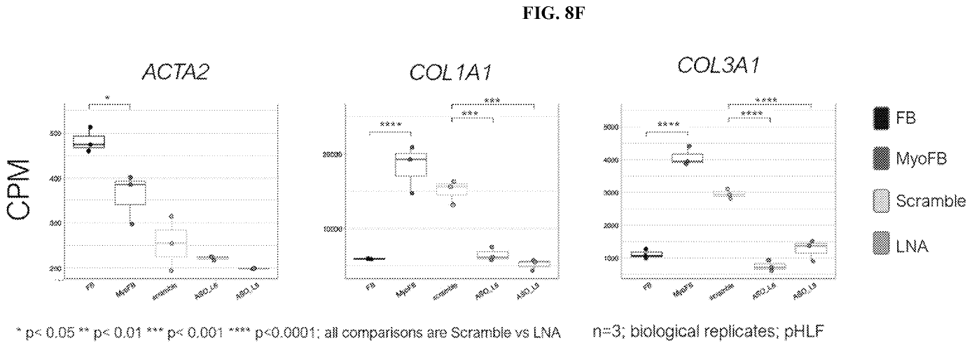

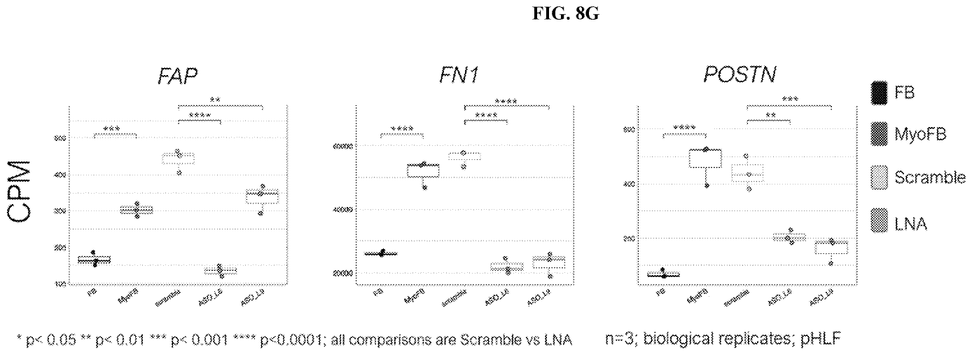

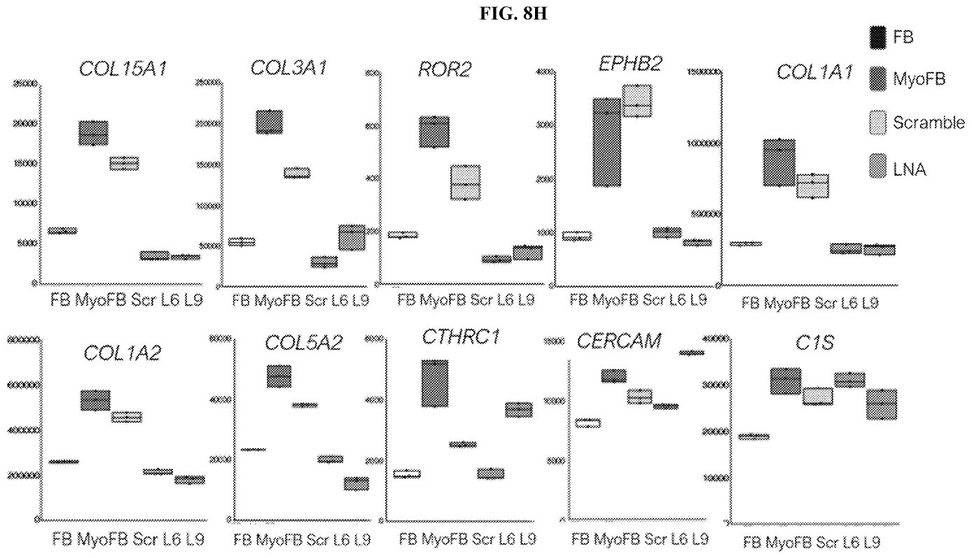

FIG. 8A shows an overview of PUFIN7 and target location of designed ASOs (LNA1-LNA9). As shown in FIGS. 8B-8G, PUFIN7 expression level was decreased when myofibroblasts were treated with LNA ASOs as shown in Table 27 relative to the sample treatment with scramble ASO. Further, gene expression of canonical markers of fibrosis (COL1A1, FN1, POSTN, FAP, ACTA2, CTHRC1, and COL3A1) were decreased when myofibroblasts was treated with LNA ASOs, e.g., LNA6 (or L6) (TGAAGTAGCCATAGCC (SEQ ID NO: 32)) or LNA9 (or L9) (GTCACCACATGTTAGT (SEQ ID NO: 35)) compared to expression of myofibroblasts treated with scramble ASO (FIGS. 8C, 8F-8G).

### LNA Knockdown of PUFIN7 Downregulated Differentially Expressed Genes

Genome wide differential expression and IPF gene score (Singscore) were affected when PUFIN7 expression was suppressed by treatment with ASOs (LNA6 or LNA9). Examples of genes affected by PUFIN7 downregulation were COL3A1, FN1, FAP, COL1A1, POSTN, LINCO1614, ACTA2, ENSG00000223786, ENSG00000259345, ENGS00000289423, ENSG00000226526, ENSG00000249406, XLOC_025418, and ZNF295-AS1 (data not shown). Upon LNA6 treatment, 1425 genes were downregulated, and 2933 genes were upregulated compared to scramble control. Upon LNA9 treatment, 963 genes were downregulated, and 2898 genes were upregulated compared to scramble control (data not shown). GSEA analysis of pathways downregulated by LNA6 treatment comprised regulation of cell organization, regulation of actin filament organized, establishment or maintenance of polarity, and other ECM organizational related pathways. The top 50 downregulated pathways were Golgi vesicle transport; positive regulation of cell projection organization; regulation of actin filament organization; establishment or maintenance of cell polarity; actin polymerization or depolymerization; glycerophospholipid metabolic process; synapse organization; developmental growth involved in morphogenesis; regulation of small GTPase mediated signal transduction; positive regulation of nervous system development; regulation of neuron projection development; regulation of cellular component size; regulation of actin cytoskeleton organization; regulation of actin filament-based process; cell-substrate adhesion; phospholipid metabolic process; regulation of GTPase activity; regulation of supramolecular fiber organization; positive regulation of cell development; small GTPase mediated signal transduction; regulation of cell morphogenesis; glycoprotein biosynthetic process; extracellular matrix organization; cilium assembly; glycerolipid metabolic process; external encapsulating structure organization; extracellular structure organization; protein localization to plasma membrane; positive regulation of GTPase activity; regulation of neurogenesis; vesicle organization; protein localization to cell periphery; glycoprotein metabolic process; regulation of nervous system development; cilium organization; actin filament organization; regulation of anatomical structure size; positive regulation of cellular component biogenesis; axon development; axonogenesis; cell junction assembly; negative regulation of organelle organization; negative regulation of locomotion; regulation of cell development; skeletal system development; regulation of Wnt signaling pathway; response to peptide; muscle cell differentiation; regulation of protein-containing complex assembly; positive regulation of cell migration; ameboidal-type cell migration; positive regulation of organelle organization; Wnt signaling pathway; cell-cell signaling by wnt; morphogenesis of an epithelium; wound healing; modulation of chemical synaptic transmission; regulation of trans-synaptic signaling; and calcium ion transport. The top pathways that were upregulated were related to genome regulation. The top 50 upregulated pathways were positive regulation of striated muscle contraction; negative regulation of necroptotic process; DNA replication-dependent chromatin assembly; protection from non-homologous end joining at telomere; maturation of SSU-rRNA; negative regulation of programmed necrotic cell death; DNA replication checkpoint signaling; ribosomal small subunit biogenesis; ribosomal small subunit assembly; ribosome assembly; regulation of DNA-templated DNA replication initiation; response to UV-B; DNA replication initiation; chaperone cofactor-dependent protein refolding; ‘de novo’ post-translational protein folding; ‘de novo’ protein folding; regulation of p38MAPK cascade; blastocyst formation; ribosome biogenesis; nuclear DNA replication; nucleosome assembly; transcription elongation by RNA polymerase II promoter; rRNA processing; ribonucleoprotein complex biogenesis; rRNA metabolic process; protein-DNA complex assembly; regulation of transcription elongation by RNA polymerase II; nucleosome organization; regulation of DNA-templated transcription elongation; intrinsic apoptotic signaling pathway in response to DNA damage; mismatch repair; DNA integrity checkpoint signaling; keratinization; transcription initiation at RNA polymerase II promoter; DNA replication; mitochondria translation; ribonucleoprotein complex assembly; DNA damage checkpoint signaling; DNA-templated DNA replication; response to UV; mitochondrial gene expression; ncRNA processing; response to topologically incorrect protein; protein folding; protein-DNA complex subunit organization; RNA phosphodiester bond hydrolysis; DNA-templated transcription initiation; ribonucleoprotein complex subunit organization; cell cycle checkpoint signaling; tRNA metabolic process; telomere organization; nucleic acid phosphodiester bond hydrolysis; DNA recombination; negative regulation of cell cycle; and regulation of response to DNA damage stimulus. Genes associated with PUFIN7 co-expression module were, for examples, CERCAM, COL3A1, COL1A1, COL5A2, COL1A2, CIS, ROR2, EPHB2, COL15A1, and CTHRC1. The top pathways that were downregulated by LNA9 were dominated by ECM and cellular organization pathway, and these top downregulated pathways comprised cell-matrix adhesion, regulation of Ras protein signal transduction, regulation of cell morphogenesis, and several GTPase activity pathways. The top 50 downregulated pathways by LNA9 were regulation of small GTPase mediated signal transduction; cell-matrix adhesion; phospholipid biosynthetic process; regulation of Ras protein signal transduction; regulation of cell morphogenesis; regulation of GTPase activity; actin polymerization or depolymerization; cilium assembly; establishment or maintenance of cell polarity; positive regulation of GTPase activity; small GTPase mediated signal transduction; glycerophospholipid biosynthetic process; external encapsulating structure organization; extracellular matrix organization; regulation of actin filament organization; extracellular structure organization; positive regulation of cell projection organization; cilium organization; regulation of supramolecular fiber organization; negative regulation of organelle organization; Golgi vesicle transport; phospholipid metabolic process; protein localization to plasma membrane; regulation of actin cytoskeleton organization; Ras protein signal transduction; glycerolipid metabolic process; glycerolipid biosynthetic process; actin filament organization; protein localization to cell periphery; regulation of actin filament-based process; regulation of Wnt signaling pathway; neuron projection guidance; peptidyl-serine phosphorylation; axon guidance; glycerophospholipid metabolic process; dendrite development; regulation of cellular component size; regulation of neuron projection development; glycoprotein biosynthetic process; cell junction assembly; ameboidal-type cell migration; skeletal system development; glycoprotein metabolic process; Wnt signaling pathway; cell-cell signaling by Wnt; dephosphorylation; regulation of protein-containing complex assembly; positive regulation of cellular component biogenesis; positive regulation of cell migration; synapse organization; anoxogenesis; regulation of anatomical structure size; axon development; positive regulation of kinase activity; regulation of nervous system development; regulation of vesicle-mediated transport; morphogenesis of an epithelium; regulation of cell development; and angiogenesis. The top upregulated pathways by LNA9 were related to genome regulation. The top 50 upregulated pathway by LNA9 were U2-type prespliceosome assembly; protein refolding; maturation of SSU-rRNA from tricistronic rRNA transcript (SSU-rRNA, 5.8S rRNA, LSU-rRNA); ribosomal small subunit biogenesis; maturation of SSU-rRNA; ribosomal large subunit biogenesis; spliceosomal snRNP assembly; rRNA processing; ribosome biogenesis; ribosome assembly; rRNA metabolic process; cytoplasmic translation; ribonucleoprotein complex biogenesis; protein insertion into membrane; spliceosomal complex assembly; chaperone-mediated protein folding; ribonucleoprotein complex assembly; RNA splicing, via transesterification reactions; mRNA splicing, via spliceosome; RNA splicing, via transesterification reactions with bulged adenosine as nucleophile; ncRNA processing; ribonucleoprotein complex subunit organization; mitochondrial translation; nucleosome assembly; RNA splicing; tRNA processing; positive regulation of mRNA metabolic process; response to type I interferon; mitochondrial gene expression; nucleosome organization; oxidative phosphorylation; protein folding; translational initiation; regulation of RNA splicing; RNA modification; protein-DNA complex assembly; negative regulation of response to biotic stimulus; protein-DNA complex subunit organization; negative regulation of translation; RNA localization; negative regulation of cellular macromolecule biosynthetic process; negative regulation of cellular amide metabolic process; regulation of viral process; RNA catabolic process; regulation of translation; regulation of cellular amide metabolic process; response to virus; defense response to virus; defense response to symbiont; nucleobase-containing compound catabolic process; viral process; mitochondrion organization; regulation of cellular macromolecule biosynthetic process; heterocycle catabolic process; establishment of protein localization to organelle; intrinsic apoptotic signaling pathway; organic cyclic compound catabolic process; cellular response to lipopolysaccharide; and cellular response to molecule of bacterial origin. Knockdown of PUFIN7 by both LNA6 and LNA9 showed a certain cohesive pattern of gene pathway down regulation—both LNAs effect cell organization, wound healing, and ECM related pathways (data not shown). As shown in FIG. 8E, IPF gene score (Singscore) showed a decrease in fibrotic gene expression upon treatment with LNA ASOs, either LNA6 or LNA9, that target PUFIN7.

PUFIN7 was Expressed within a Collagen Network Module and Treatment of ASO Targeting PUFIN7 Downregulated Gene Expression in this ECM Gene Network

Network analysis of IPF in vivo data produced 41 total clusters and 11 merged clusters. PUFIN7 and PUFIN3 were both within the same enriched network pathway (same network module), and the enriched pathways in this network module were extracellular structure organization, cell-substrate adhesion, cell junction assembly, Wnt signaling, wound healing, heart contraction, collagen metabolic process, collagen fibril organization, and extracellular matrix disassembly. As shown in FIGS. 8H and 8I, upon ASO treatment, e.g., LNA6 (L6) or LNA9 (L9), top 10 genes that were co-expressed with PUFIN7 expression were COL15A1, COL3A1, ROR2, EPHB2, COL1A1, COL1A2, COL5A2, CTHRC1, CERCAM, CIS. Upon PUFIN7 ASO treatment (either LNA6 or LNA9 compared to scramble), significant proportion of gene expression in the same network pathway, were downregulated (data not shown). Common enriched pathways were extracellular structure organization; cell-substrate adhesion; cell junction assembly; Wnt signaling pathway; wound healing; heart contraction; collagen metabolic process; collagen fibril organization; and extracellular matrix disassembly.

Further, network analysis results indicated that PUFIN7 and PUFIN3 play roles in the same enriched network pathway, as being co-expressed with various ECM related genes. Top enriched pathways in this shared network hinted at the potential role of these PUFINs in various biological processes such as extracellular structure organization, wound healing, and collagen metabolic processes, all of which are crucial in fibrotic disease processes. For example, the knockdown of PUFIN7 affected many ECM genes (~18% of the total ECM associated network), indicating PUFIN7's potential role as a regulator in the gene expression of this network. Such collagen and ECM based gene signatures have shown a robust ability to predict IPF severity and mortality.

Further, genes associated with PUFIN3 co-expression network were, for examples, NTN1, TMEM45A, GOLM1, SERPINB5, FAT2, and SPTBN2. When PUFIN7 was knocked down with LNA ASOs (LNA6 or LNA9), the gene expression in this network was significantly downregulated. LNA6 downregulated 18% of gene expression in the network (Fisher's test p value=1.21069e-³⁰) and LNA9 downregulated 16% of gene expression in the network compared to scramble control (Fisher's test p-value=4.032515e-²⁴).

ScRNAseq data analysis using the database by Adams et al., Sci Adv (2020) and Habermann, A. et al. Sci Adv. (2020) provided that the expression of PUFIN7 was higher in IPF patient samples than COPD patient samples, and the expression was generally low in heathy controls. Further, in the cellular level, PUFIN7 expression was substantially high in the myofibroblasts in IPF patients (data not shown). This result indicates that expression of PUFIN7 is associated with IPF pathology or prognosis, and pulmonary fibrosis development.

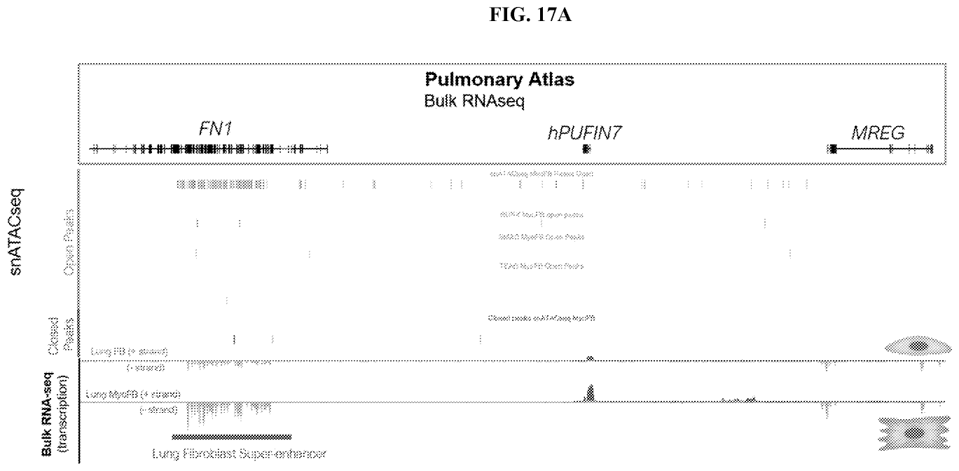

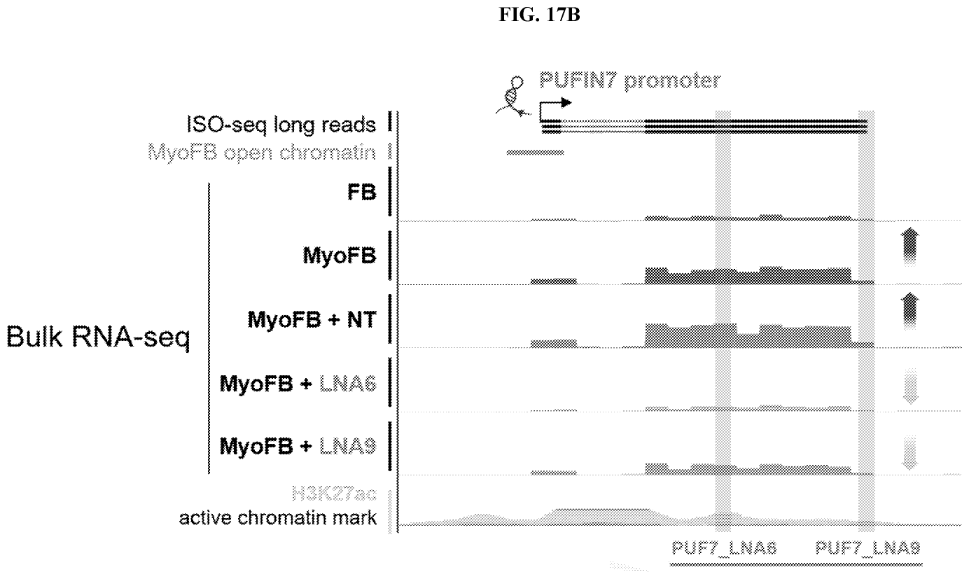

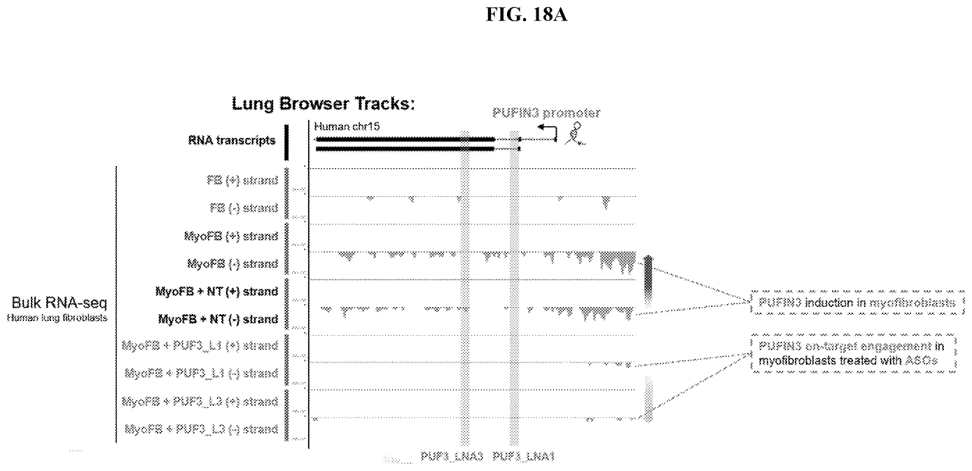

As shown in FIG. 17A, genomic locus of PUFIN7 was identified as being near fibronectin (FN1), which is associated with fibrosis development. Long read sequencing of PUFIN7 locus and bulk RNA-seq identified druggable exon of PUFIN7 that was upregulated in myofibroblasts. FIG. 17B shows that expression of PUFIN7 was enriched in lung myofibroblasts and the expression of PUFIN 7 was decreased upon LNA6 (PUF7_LNA6) (SEQ ID NO: 32) and PUFIN7 LNA9 (PUF7_LNA9) (SEQ ID NO: 35) treatment. In FIG. 17B, open chromatin peak (measured by snATAC-seq) was a predicted cis-regulatory region in myofibroblasts. H3K27ac signal highlighted a regulatory region at PUFIN7 promoter.

Overall, these results showed that ASOs targeting PUFIN7 were highly effective to downregulate PUFIN7 expression, and upon ASO knockdown of PUFIN7, a significant reduction in myofibroblast-specific or -enriched gene expression was observed. These results provided evidence of the potential utility of ASO targeting PUFIN7 as a therapeutic strategy to treat IPF or IPF symptoms. Further, these results provided important insights into the cellular and molecular mechanisms underlying IPF and highlight the potential of using ASOs targeting PUFIN7 as a therapeutic strategy to treat IPF.

### TEP Study of ASO Targeting PUFIN7

In vitro dosage response experiment using PUFIN7 L6 ASO (SEQ ID NO: 32) was performed as described in the previous example.

### Methods:

In vitro cell culture methods and experiments were performed as described in previous examples. The effect of L6 ASO (SEQ ID NO: 32) to modulate various downstream gene expression was examined at concentrations of 0.5 nM, 1 nM, 5 nM, 10 nM, 20 nM, and 50 nM.

### Results:

Results from this experiment were used to cluster RNA transcripts showing dose-dependent expression where the lncRNA (PUFIN7) was targeted by L6 ASO (SEQ ID NO: 32). RNA extraction, qPCR, and bulk RNAseq were performed as described in the previous examples. Expression of these dose-dependent genes were clustered to form a Target Engagement Panel (TEP).

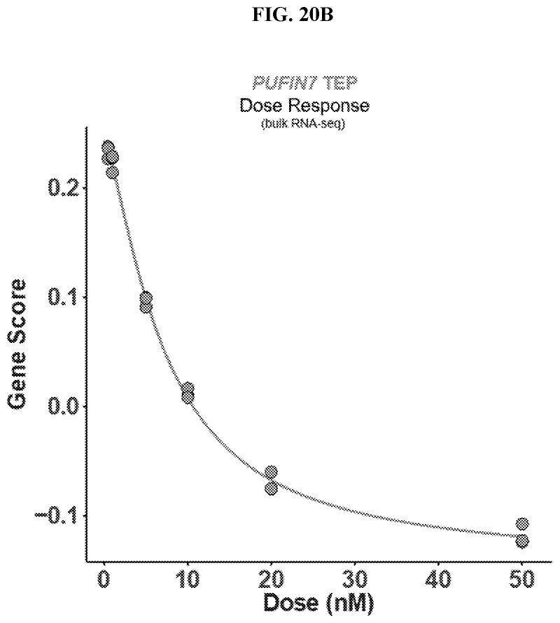

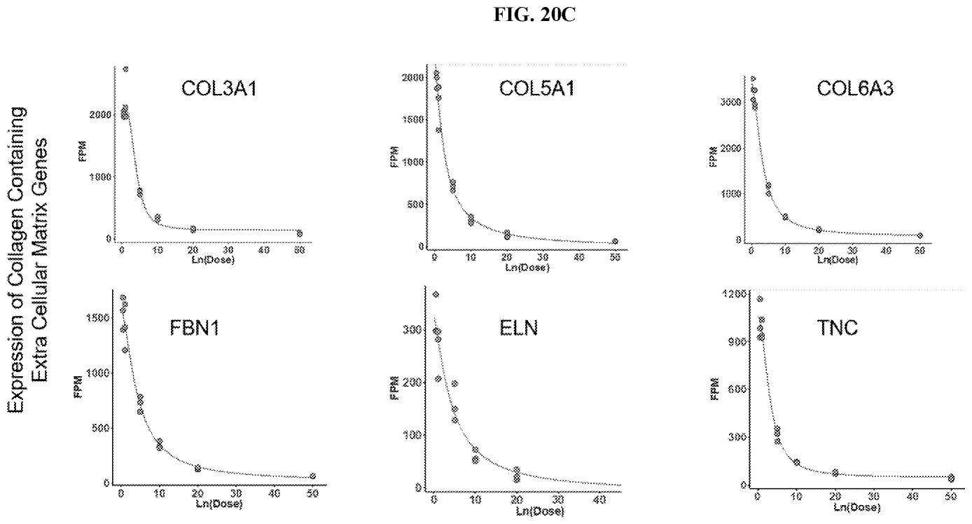

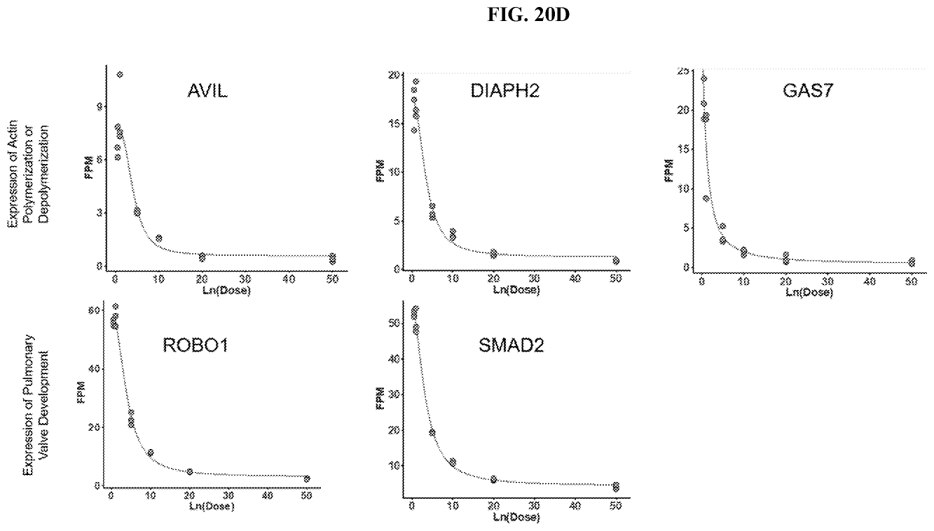

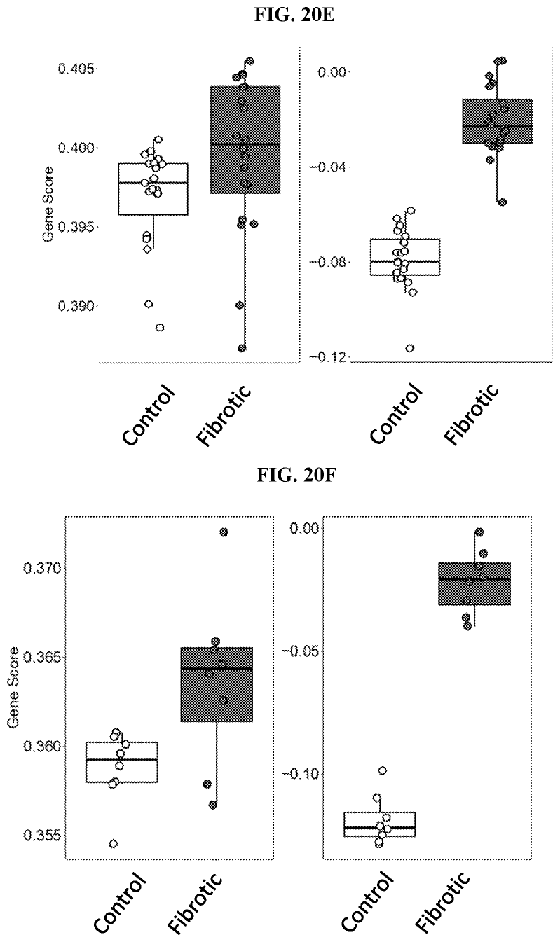

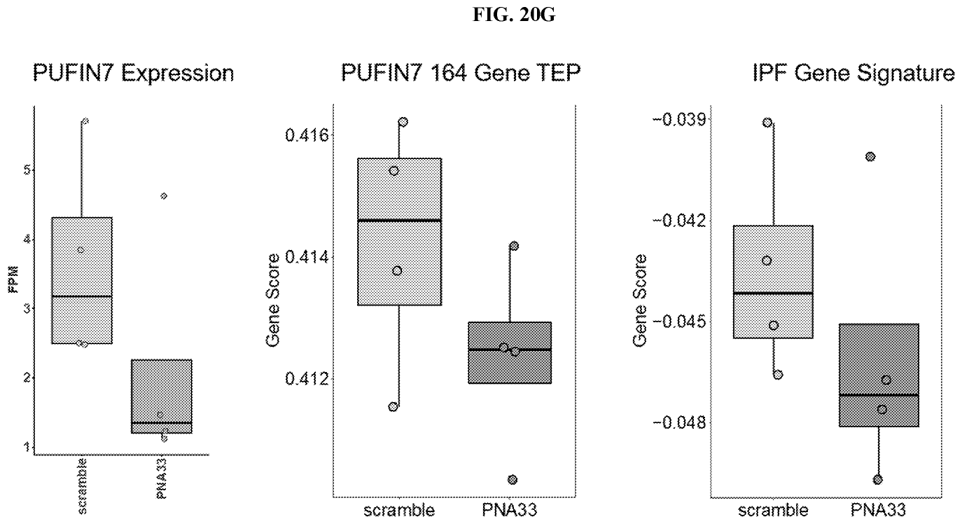

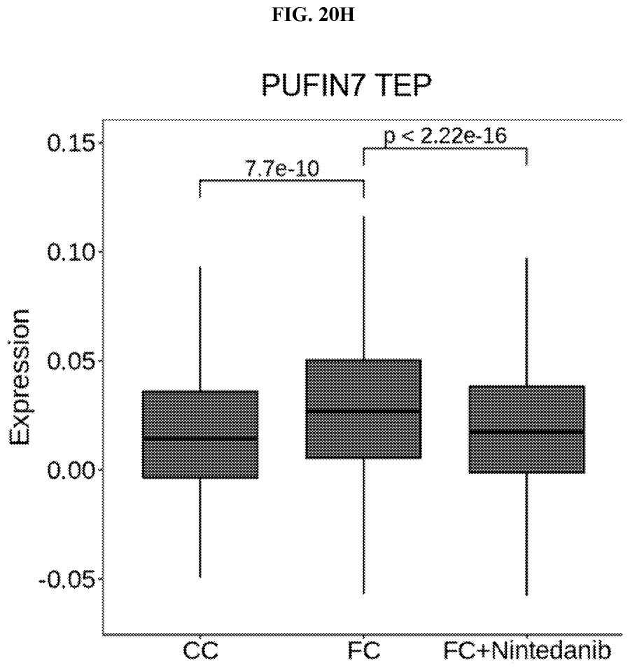

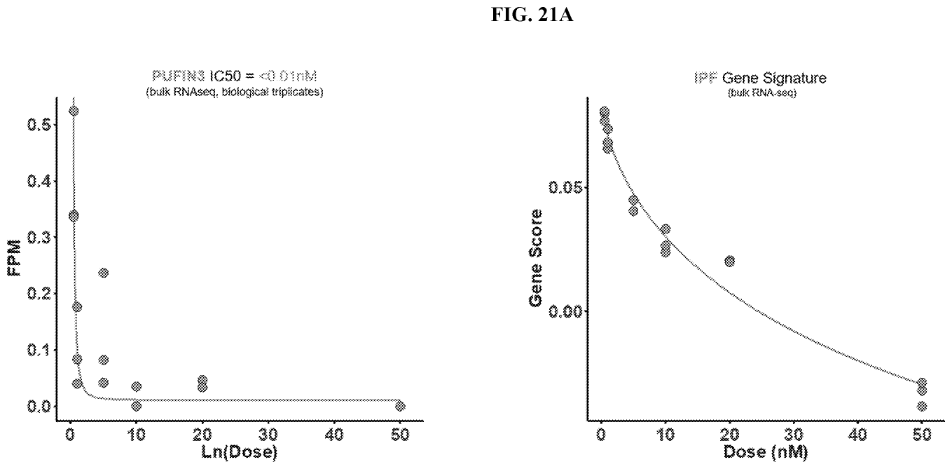

FIG. 20A shows that PUFIN7 expression was a dose-dependent upon L6 ASO (SEQ ID NO: 32) treatment. Also, expression of IPF gene signature (genes listed in Table 18) was downregulated in a dose-dependent manner upon L6 ASO treatment. Dose-responsive genes were identified and further clustered into PUFIN7 Target Engagement Panel (TEP). First pass clustering resulted in initial 7 gene clusters. Clusters 1 and 4 from this step were further reclustered for TEP refinement. The second clustering step resulted in 7 gene clusters, and cluster 3 of the second clustering step comprised the most responsive genes. Genes in cluster 3 were identified as PUFIN7 TEP, which comprises 164 genes. List of genes in PUFIN7 TEP are shown in Table 32. FIG. 20B shows dose response curve calculated from 164 dose-responsive TEP genes upon L6 ASO (SEQ ID NO: 32) treatment. Top GO pathways associated with genes in PUFIN7 TEP were genes associated with collagen-containing extracellular matrix comprising Collagen type III alpha 1 chain (COL3A1), Collagen type V alpha 1 chain (COL5A1), Collagen type VI alpha 3 chain (COL6A3), Elastin (ELN), Tenascin C (TNC), MXRA5, Fibrillin 1 (FBN1); genes associated with actin polymerization or depolymerization comprising Diaphanous related formin 2 (DIAPH2), Advillin (AVIL), Growth arrest specific 7 (GAS7); and genes associated with pulmonary valve development comprising SMAD family member 2 (SMAD2), Roundabout guidance receptor 1 (ROBO1). As shown in FIG. 20C, expression of IPF related genes comprising collagen-containing extracellular matrix, e.g., COL3A1, COL5A1, COL6A3, ELN, TNC, and FBN1, responded in a dose dependent manner upon PUFIN7 L6 ASO treatment. As shown in FIG. 20D, expression of additional genes, e.g., AVIL, DIAPH2, GAS7, ROBO1, and SMAD2, responded in a dose dependent manner upon PUFIN7 L6 ASO treatment.

TABLE 32
Genes in the PUFIN7 TEP.

| | | | | |
|---|---|---|---|---|
| AAK1 | DNAJC8 | JAM3 | NXPH3 | SLC66A2 |
| ADAM12 | DYM | JAZF1 | OSBPL3 | SLC7A11 |
| ADAMTS13 | DYSF | KCNH1 | PACS1 | SLC7A6 |
| ADM2 | ECI2 | KCNT2 | PAPPA2 | SMAD2 |
| ADORA1 | EDA2R | KDM3A | PCDH1 | SMARCAD1 |
| AGFG1 | EHBP1 | KIF1B | PCNX1 | SMYD3 |
| AGK | ELN | KLF12 | PHF8 | SND1 |
| ANKS1A | ENOX1 | LIN7A | PKNOX2 | SNTB1 |
| AREL1 | ENOX2 | LINC00968 | PLCB4 | SNX29 |
| ARHGAP42 | ENSG00000253317 | LINC01239 | POLR3B | SPATA20 |
| ARID2 | ERI3 | LINC02544 | PPFIBP1 | STAT3 |
| ARMC8 | ETV6 | LPP | PPP1R11 | STEAP3 |
| ASTN2 | EXOC4 | LSAMP | PREP | SUPT6H |
| AVIL | EXT1 | MAMDC2 | PRRC2B | SYPL2 |
| BPHL | FAM114A2 | MAPKAPK2 | RABGAP1 | SYT7 |
| BTBD9 | FBN1 | MAST2 | RABGAP1L | TANGO6 |
| CAB39 | FBXL7 | MB21D2 | RALGAPA2 | TFEB |
| CADPS | FBXO41 | MINDY3 | RBM33 | TMEM167A |
| CALD1 | FOXN3 | MOGS | ROBO1 | TMEM168 |
| CAMK1D | GAS6-DT | MPDZ | RPS6KC1 | TNC |
| CCDC183-AS1 | GAS7 | MSRB3 | RSAD2 | TRMT9B |
| CCT3 | GATA6 | MTX3 | SBNO2 | TTC28 |
| CEP170B | GLCE | MX2 | SCAMP1-AS1 | TVP23B |
| CEPT1 | GNB1L | MXRA5 | SCRN1 | USP3 |
| CIZ1 | GOLGA6L5P | NAV2 | SEC23IP | USP30 |
| CLCN5 | GOLGA8A | NEGR1 | SEC61A1 | VRK2 |
| COL3A1 | GOLGA8B | NEK10 | SGCD | WDR48 |
| COL5A1 | HECTD2 | NFYA | SH3PXD2A | XLOC_063910 |
| COL6A3 | HPCAL1 | NHLRC2 | SKAP2 | XLOC_068687 |
| COMMD 10 | HYDIN2 | NLK | SLC12A9 | XLOC_091557 |
| DDX1 | ITPR1 | NME7 | SLC39A11 | ZCCHC7 |
| DENND1A | JADE2 | NTM | SLC41A2 | ZRANB2 |
| DIAPH2 | JADE3 | NUP133 | SLC44A1 | |

Expression of gene sets of PUFIN7 TEP was validated using the data available in public bulk RNA-seq database. FIGS. 20E and 20F show that expression of gene sets of PUFIN7 TEP correlated with human IPF signature (genes listed in Table 18) based on analysis of RNA-seq data reported by Schafer et al., Nat Commun. (2017) (FIG. 20E) and Luzina et al., Cell Immunol. (2018) (FIG. 20F). These results suggest that genes of PUFIN7 TEP were highly expressed in IPF patients and that targeting PUFIN7 provides potential approach to modulate disease or revert disease transcription.

Results of bulk RNA-seq heatmap of the top 100 most downregulated genes based on PUFIN7 L6 ASO dose response showed that these downregulated genes displayed dose-responsive decrease associated with PUFIN7 L6 ASO treatment (data not shown). Protein-protein interaction network was reconstructed using genes in PUFIN7 TEP. Protein-protein functional interaction network of genes in PUFIN7 TEP was generated by STRING Database analysis, and genes that were shown to interact with each other were shown in Tables 38-39. The PUFIN7 TEP Network was connected by key genes that regulated IPF disease. This network was dominated by an interconnected set of collagen genes.

TABLE 38
Genes in PUFIN7 TEP that were predicted to have protein-protein functional
interaction.

| | | | | | |
|---|---|---|---|---|---|
| NLK | COL3A1 | NUP133 | VRK2 | PCDH1 | SEC61A1 |
| MAMDC2 | MFAP5 | HECTD2 | PCNX1 | SLC39A11 | MAST2 |
| GAT6 | COL5A1 | FBXO41 | COMMD10 | SCRN1 | VIRMA |
| GLCE | SMAD2 | SMARCAD1 | SLC66A2 | LPP | AS7 |
| SNX29 | JADE2 | ZRANB2 | TMEM167A | SYT7 | ASTN2 |
| MFAP2 | JADE3 | RABGAP1L | RALGAPA2 | NEK10 | SH3PXDA2 |
| LOXL2 | PREP | DDX1 | TVP23B | SBNO2 | TFEB |
| FOXN3 | DNAJC8 | GOLGA8B | BPHL | SYPL2 | CALD1 |
| PLOD3 | KDM3A | GOLGA8A | CEP170B | NXPH3 | SGCD |
| ELN | KLF12 | RBM33 | SLC41A2 | ADM2 | ADORA1 |
| EFEMP2 | EXT1 | ARMC8 | PKNOX2 | SNTB1 | SKAP2 |
| COL15A1 | MAPKAPK2 | USP3 | AVIL | CLCN5 | NTM |
| LOXL1 | MX2 | ZCCHC7 | KCNT2 | PACS1 | BTBD9 |
| ADAMTS13 | PRRC2B | TANGO6 | OSBPL3 | JAM3 | EDA2R |
| MFAP5 | RSAD2 | WDR48 | ECI2 | DYSF | PPP1R11 |
| COL5A2 | NIZ1 | DENND1A | HPCAL1 | SYDIN | ITPR1 |
| COL6A3 | ARID2 | FAM114A2 | STEAP3 | MPDZ | NHLRC2 |
| PLOD1 | CCT3 | AREL1 | ARHGAP42 | EHBP1 | DYM |
| COL16A1 | SMYD3 | MTX3 | PPFIBP1 | PAPPA2 | POLR3B |
| COL11A1 | MSRB3 | TMEM168 | KIF1B | PAPPA | EXOC4 |
| ADAMTS2 | STAT3 | MOGS | SLC12A9 | LSAMP | NME7 |
| COL4A1 | TP51 | ENOX2 | LIN7A | NAV2 | SLC7A6 |
| ROBO1 | TTC28 | CEPT1 | CAB38 | JAZF1 | SEC23IP |
| PLCB4 | ETV6 | RABGAP1L | KCNH1 | ASTN2 | |
| ADAM12 | PHF8 | AAK1 | PCDH1 | TFEB | |
| TNC | NFYA | MINDY3 | SCRN1 | NEGR1 | |
| MXRA5 | DDX1 | ENOX1 | CADPS | ANKS1A | |

TABLE 39
A subset of genes from Table 38 that were associated with
collagen and other extracellular matrix related genes.

| | | | | | |
|---|---|---|---|---|---|
| GATA6 | ELN | POSTN | MFAP2 | COLA2 | ADAM12 |
| STAT3 | MXRA5 | COL1A2 | COL5A1 | MFAP5 | ADAMTS13 |
| SMAD2 | LOXL1 | COL6A3 | LTBP2 | ADAMTS2 | |

Transcription factor enrichment analysis in genes of PUFIN7 TEP was performed as described in the previous example, and results show that ESR1, NR3C1, TEAD4, CTNNB1, SMAD4, STAT3, SMARCD1, WT1, TOP2B, and PPARD were among the top transcription factors that regulated 164 genes in PUFIN7 TEP. ENRICHR analysis of top Reactome Gene Ontology Pathways associated with genes in PUFIN7 TEP was performed, and results show that top pathways were signaling by PDGFR in disease; transcriptional regulation of pluripotent stem cells; collagen chain trimerization; RHOD GTPase cycle; extracellular matrix organization; assembly of collagen fibrils and other multimeric structures; chromatin modifying enzymes; signaling by BRAF and RAF1 fusions; collagen biosynthesis and modifying enzymes; and basigin interactions. ENRICHR analysis of top Wikipathways associated with genes in PUFIN7 TEP was also performed and results show that these pathways were miRNA targets in ECM and membrane receptors; serotonin HTR1 group and FOS pathway; endoderm differentiation; TGF beta signaling in thyroid cells for epithelial mesenchymal transition; miR509 3P alteration of YAP1 ECM axis; MFAP5 effect on permeability and motility of endothelial cells via cytoskeleton rearrangement; autophagy in pancreatic ductal adenocarcinoma; serotonin receptor 2 and ELK SRF GATA4 signaling; TGF beta receptor signaling in skeletal dysplasias; and progeria associated lipodystrophy. Gene Ontology (GO) Pathway Analysis based on GO Molecular Processes, GO Cellular Components, and GO Biological Processes, revealing variety of cellular, molecular, and biological pathways associated with genes in PUFIN7 TEP. Top pathways in GO molecular processes comprised platelet-derived growth factor binding; histone H3K9 demethylase activity; antiporter activity; calmodulin-dependent protein kinase activity; histone demethylase activity; metal ion transmembrane transporter activity; calcium ion binding; chromatin DNA binding; phosphatidylinositol bisphosphate binding; and 2-oxoglutarate-dependent dioxygenase activity. Top pathways in GO cellular components comprised nuclear membrane; mitochondrial outer membrane; protein kinase complex; organelle outer membrane; histone acetyltransferase complex; actin filament; polymeric cytoskeletal fiber; endoplasmic reticulum lumen; collagen-containing extracellular matrix; supramolecular fiber. Top pathways in GO biological processes comprised eye morphogenesis; establishment of protein localization to extracellular region; histone H4-K12 acetylation; protein secretion; aortic valve morphogenesis; macrophage activation involved in immune response; aortic valve development; exocytosis; histone H4-K5 acetylation; histone H4-K8 acetylation. Based on analysis using MSigDB, whose result is shown in Table 40 below, genes in PUFIN7 TEP were highly associated with EMT. Key genes identified were COL3A1, SGCD, COL6A1, ADAM12, COL5A1, CALD1, ELN, FXBL7, TNC, and FBN1.

TABLE 40
Pathways associated with genes from the PUFIN7 TEP
based on human molecular signatures database.

| | | | | | Com- |
|---|---|---|---|---|---|
| In- | | | Adjusted | Odds | bined |
| dex | Name | P-value | p-value | Ratio | score |
| 1 | Epithelial | 9.97E−08 | 0.000003291 | 8.25 | 133.01 |
| | mesenchymal | | | | |
| | transition | | | | |
| 2 | myogenesis | 0.006203 | 0.06824 | 3.84 | 19.54 |
| 3 | apical junction | 0.006203 | 0.06824 | 3.84 | 19.54 |
| 4 | spermatogenesis | 0.09963 | 0.7463 | 2.78 | 6.41 |
| 5 | protein secretion | 0.1861 | 0.7463 | 2.59 | 4.36 |
| 6 | PI3K/AKT/ | 0.2129 | 0.7463 | 2.37 | 3.66 |
| | mTOR signaling | | | | |
| 7 | hypoxia | 0.2262 | 0.7463 | 1.86 | 2.76 |
| 8 | interferon gamma | 0.2262 | 0.7463 | 1.86 | 2.76 |
| | response | | | | |
| 9 | glycolysis | 0.2262 | 0.7463 | 1.86 | 2.76 |
| 10 | p53 pathway | 0.2262 | 0.7463 | 1.86 | 2.76 |

As shown in FIG. 20G, which was analyzed from public bulk RNAseq data (Ahangari et al., JCI Insight. (2023)), PUFIN7 expression was predicted to decrease in an ex vivo human lung tissue model after post-fibrotic treatment, e.g., PNA33. Expression of genes in PUFIN7 TEP and expression of genes in IPF gene signatures were also predicted to decrease in this dataset. This result shows that targeting PUFIN7 can have anti-fibrotic effects.

Based on analysis of scRNAseq results of ex vivo model reported by Lang et al., Sci Tran Med. (2024), genes in PUFIN7 TEP and in IPF gene signature showed highest expression in myofibroblasts (data not shown). In this example, three ex vivo conditions were tested: control cocktail (CC), fibrotic cocktail (FC), and fibrotic cocktail+Nintedanib (FC+Nintedanib). FIG. 20H shows that expression of genes in PUFIN7 TEP was increased upon FC treatment and was decreased upon anti-fibrotic treatment (FC treatment along with Nintedanib), suggesting that genes in PUFIN7 TEP can be used to predict anti-fibrotic effects and/or efficacy of a drug for treating fibrosis.

Overall, PUFIN7 TEP can be used to investigate PUFIN1 mechanism of action (MoA) or to access or compare ASO potency and efficacy.

### Example 4: ASO Knockdown of PUFIN3 Attenuated Myofibroblast Gene Expression

In this example, ASO sequences were developed in silico to target the lncRNA, PUFIN3, as identified in the Example 1.

Methods of ASO design and generation as well as experiments were performed as described in the Example 2.

Antisense oligonucleotides (ASOs) comprising locked nucleic acid (LNA) modifications were designed and generated. The ASO sequences generated in silico to target PUFIN3 can be found in Table 8. ASO sequences targeting PUFIN3 that were selected for further testing are shown in Table 2 or Table 28.

### Results:

### ASO Knockdown of PUFIN3 Attenuated Myofibroblast Gene Expression

Table 28 shows modified ASO sequences targeting PUFIN3 with LNA.

TABLE 28
Sequence of ASOs targeting PUFIN3.

| | SEQ | | |
|---|---|---|---|
| Figure | ID | Sequence | Modifi- |
| Legends | NO: | (from 5′ to 3′) | cation |
| Scr/Scramble | 2567 | AACACGTCTATACGC | LNA |
| L1 | 10 | GTCAGATCCTTGGTAT | LNA |
| L2 | 11 | GATCATTGTAGCAGAC | LNA |
| L3 | 12 | GTGTCAAGGGTAAGTC | LNA |
| L4 | 13 | GGTAGTGTTTAGGTAC | LNA |
| L5 | 14 | CCATTAGTAGACCATG | LNA |
| L6 | 15 | CTATCTTAGACCCAGC | LNA |
| L7 | 16 | GCACTAATTAACCCTT | LNA |

Notes:
The L1, L2, L3, L4, L5, L6, and L7 sequences described in this table comprise 3 LNA modified nucleotides at the 5′ end and 2 LNA modified nucleotides at the 3′ end, forming 3-11-2 configuration of LNA-ASO GapmeR. Phosphorothioate linkages were incorporated between each of the nucleotides. Scramble ASO comprises 3 LNA modified nucleotides at the 5′ end and 2 LNA modified nucleotides at the 3′ end, forming 3-10-2 configuration of LNA-ASO GapmeR.

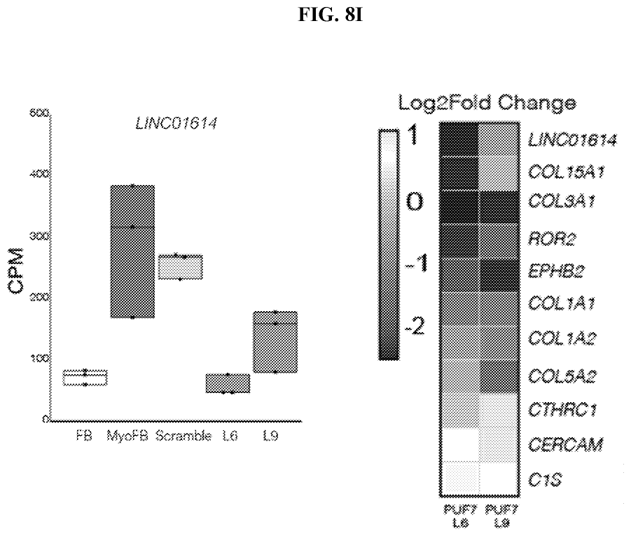

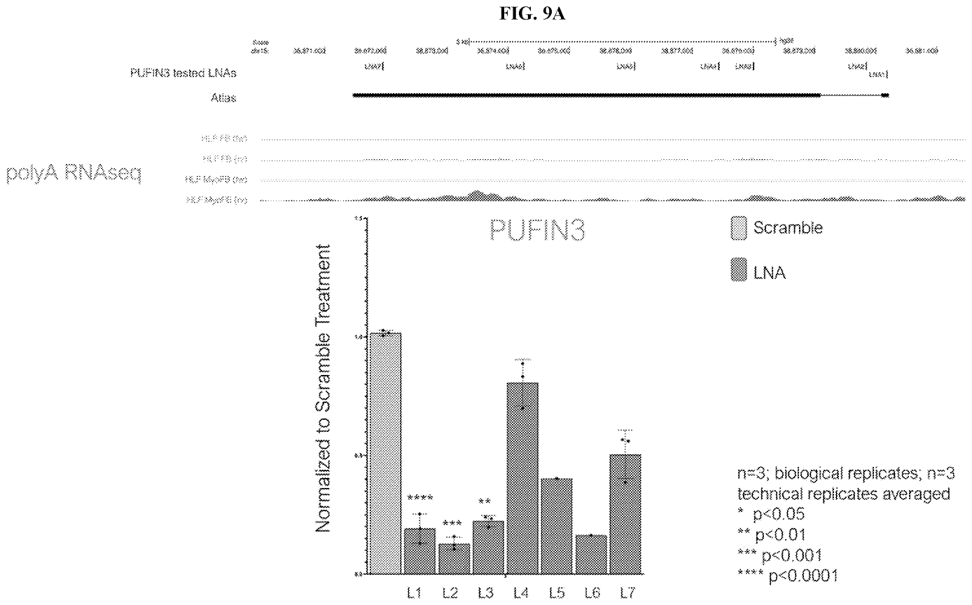

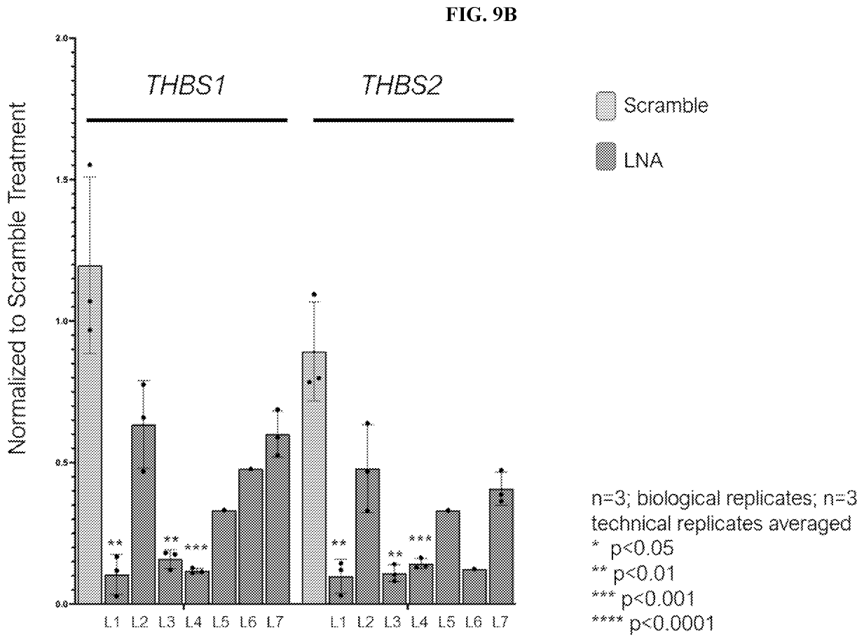

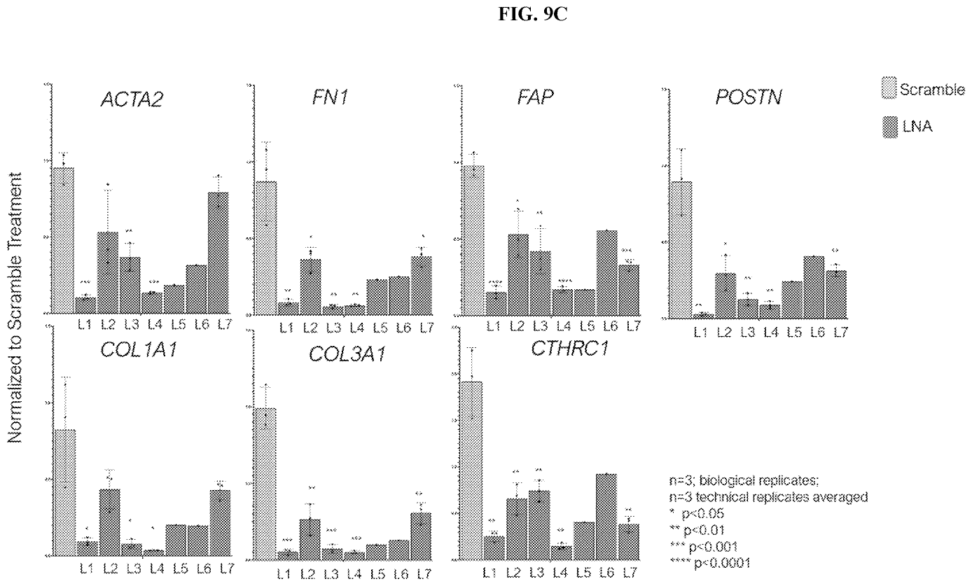

As shown in FIGS. 9A-9C, expression of PUFIN3 was decreased upon LNA ASOs treatment (results obtained from qPCR and bulk RNAseq). As shown in FIG. 9A, expression of PUFIN3 was decreased in cells treated with LNA ASOs compared to expression level of PUFIN3 from cells treated with scramble control. When cells were treated with LNA ASOs targeting PUFIN3, e.g., LNA1 or L1 (GTCAGATCCTTGGTAT (SEQ ID NO: 10)) or LNA3 or L3 (GTGTCAAGGGTAAGTC (SEQ ID NO: 12)), fibrotic genes (COL1A1, FN1, ACTA2, FAP, COL3A1, POSTN, and CTHRC1), nearest protein coding gene thrombospondin 1 (THBS1), and thrombospondin 2 (THBS2) were downregulated, which are shown in FIGS. 9B-9C

Upon lung injury, epithelial cells lose healthy cell state. An analysis of PUFIN3 expression using scRNAseq data reported by Habermann, A. et al. Sci Adv. (2020) and Adams et al., Sci Adv (2020) showed that expression of PUFIN3 increased in lung biopsies obtained from IPF patient (data not shown). Expression of PUFIN3 was upregulated in subphenotypes of epithelial cells (expressing mucin and keratin), and these cells are highly associated with IPF progression. Aberrant basal cell types are associated with honeycombing and hyperplastic basaloid formation in IPF. These abnormal formations are only present in IPF, suggesting that expression of PUFIN3 was a potential biomarker for this cell population.

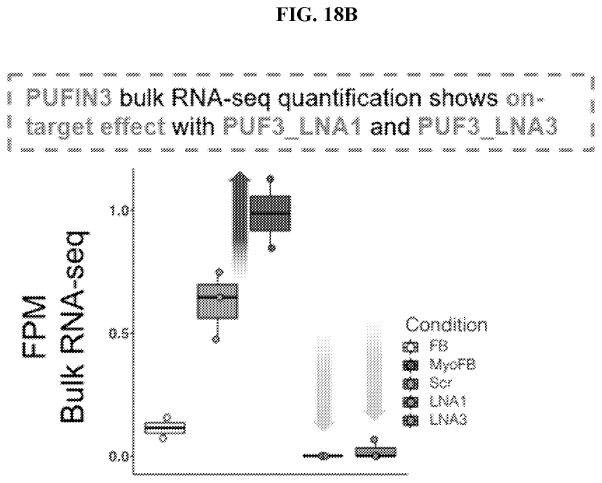

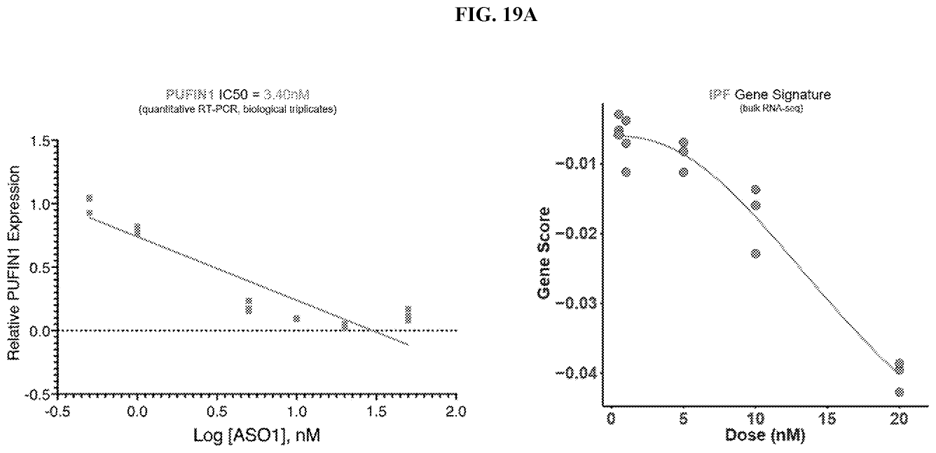

As shown in FIG. 18A, based on bulk RNA-seq analysis, PUFIN3 was transcribed from the negative (−) strand and expression of PUFIN3 was induced in myofibroblasts. Further, the expression of PUFIN3 was decreased upon PUFIN3 ASO treatment, e.g., PUFIN3 LNA3 (PUF3_LNA3) (SEQ ID NO: 12) or PUFIN3 LNA1 (PUF3_LNA1) (SEQ ID NO: 10). FIG. 18B shows expression of PUFIN3 from bulk RNA-seq, showing that PUFIN3 expression increased in myofibroblasts. Further, PUFIN3 expression decreased upon PUFIN3 LNA3 (PUF3_LNA3) (SEQ ID NO: 12) or PUFIN3 LNA1 (PUF3_LNA1) (SEQ ID NO: 10) treatment.

Overall, these results showed that ASOs targeting PUFIN3 were highly effective to downregulate PUFIN3 expression, and upon ASO knockdown of PUFIN3, a significant reduction in myofibroblast-specific or -enriched gene expression was observed. These results provided evidence of the potential utility of ASO targeting PUFIN3 as a therapeutic strategy to treat IPF or IPF symptoms. Further, these results provided important insights into the cellular and molecular mechanisms underlying IPF and highlight the potential of using ASOs targeting PUFIN3 as a therapeutic strategy to treat IPF.

### TEP Study of ASO Targeting PUFIN3

In vitro dosage response experiment using PUFIN3 LNA1 (L1) ASO (SEQ ID NO: 10) was performed as described in the previous example.

### Methods:

In vitro cell culture methods and experiments were performed as described in previous examples. The effect of. L1 ASO (SEQ ID NO: 10) to modulate various downstream gene expression was examined at concentrations of 0.5 nM, 1 nM, 5 nM, 10 nM, 20 nM, and 50 nM.

### Results:

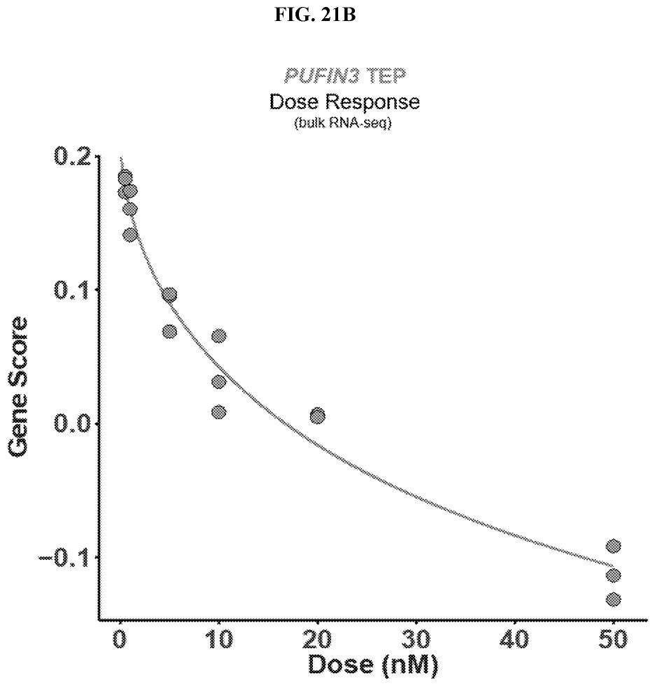

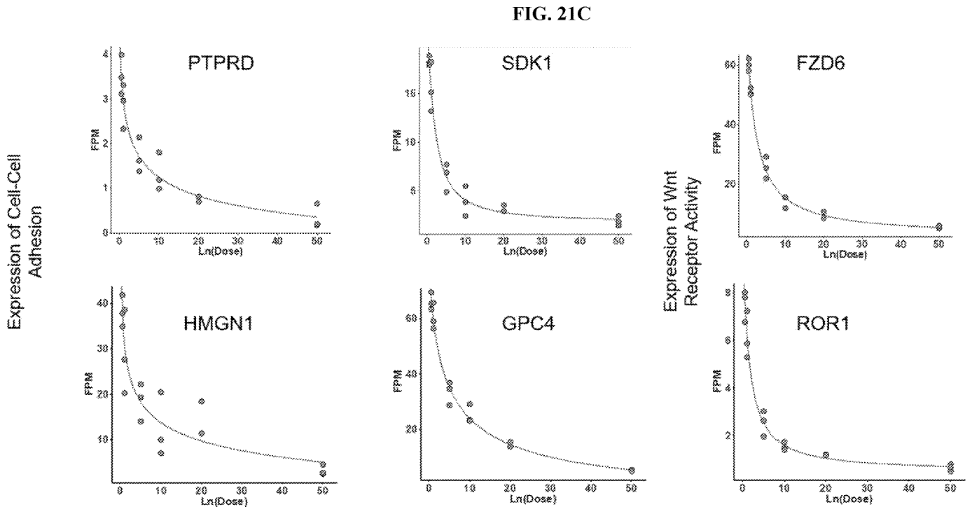

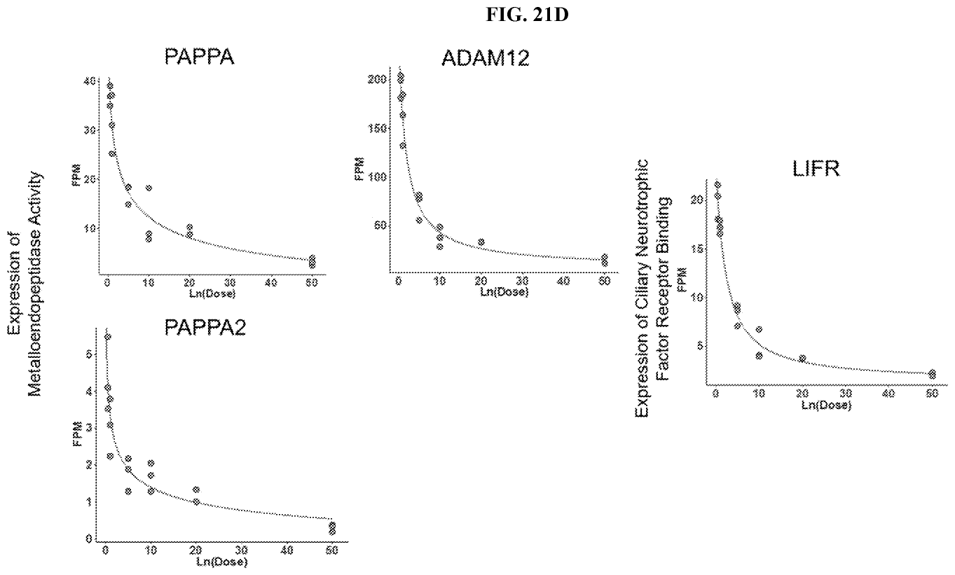

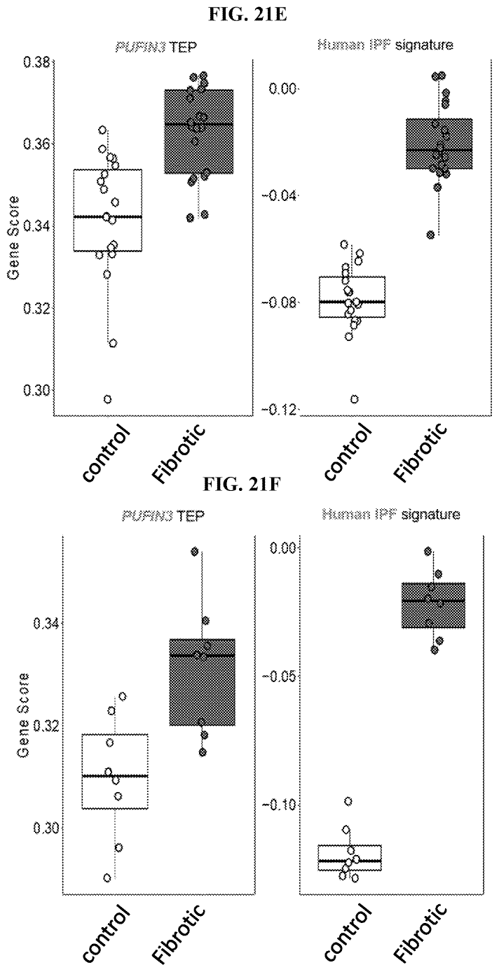

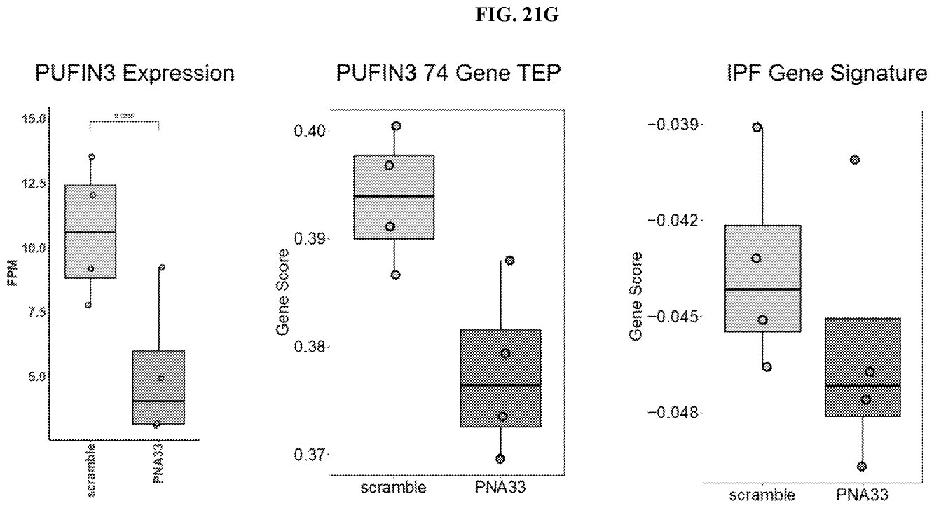

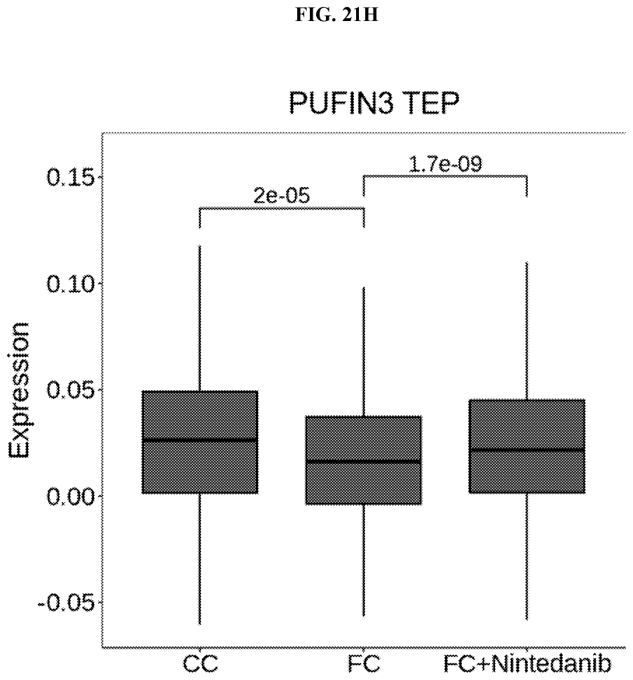

FIG. 21A shows PUFIN3 expression was a dose-dependent manner upon PUFIN3 L1 ASO (SEQ ID NO: 10) treatment. Also, expression of IPF gene signature (genes listed in Table 18) was downregulated in a dose-dependent manner upon PUFIN3 L1 ASO (SEQ ID NO: 10) treatment. Dose-responsive genes were identified and further clustered into PUFIN3 Target Engagement Panel (TEP) as described in the previous example. The first pass clustering resulted in initial 7 gene clusters. Cluster 5 from this step were further reclustered for TEP refinement. The second clustering step resulted in 5 gene clusters, and cluster 5 comprised the most responsive gene. Genes in cluster 5 were identified as PUFIN3 TEP, which comprises 74 genes. List of genes in PUFIN3 TEP are shown in Table 33. FIG. 21B shows dose response curve calculated from 74 dose-responsive TEP genes upon PUFIN 3 L1 ASO treatment. Top GO pathways associated with the PUFIN3 TEPs genes were associated with Cell-cell adhesion, e.g., Protein tyrosine phosphatase receptor type D (PTPRD), Sidekick cell adhesion molecule 1 (SDK1), High mobility group nucleosome binding domain 1 (HMGN1), Glypican 4 (GPC4), Wnt Receptor Activity, e.g., Frizzled class receptor 6 (FZD6), Receptor tyrosine kinase like orphan receptor 1 (ROR1), Ciliary neurotrophic factor receptor binding, e.g., LIF receptor subunit alpha (LIFR), and Metalloendopeptidase activity, e.g., Pappalysin 1 (PAPPA), Pappalysin 2 (PAPPA2), ADAM Metallopeptidase domain 12 (ADAM12). As shown in FIGS. 21C-21D, expression of IPF related genes that are overlapped with genes in PUFIN3 TEP responded in a dose dependent manner upon PUFIN3 L1 ASO treatment. These genes were shown to be related to cell-cell adhesion, Wnt receptor activity pathway, ciliary neurotrophic factor receptor binding, and metalloendopeptidase activity.

TABLE 33
Genes in the PUFIN3 TEP.

| | | | | | |
|---|---|---|---|---|---|
| ADAM12 | ENSG00000229160 | KIF26B | MTSS1 | PRUNE2 | TMEM132B |
| ARHGAP28 | ENSG00000277701 | KLF12 | MYH11 | PSD3 | TSHZ3-AS1 |
| BACH2 | FAM106A | LARGE1 | NAV2 | PTPRD | UNC5C |
| BRINP3 | FBXO30-DT | LIFR | OPCML | RALGPS1 | VPS13B |
| BTBD9 | FNDC1 | LINC00607 | OVCH2 | RASL11B | XLOC_90423 |
| CACNA1C | FZD6 | LINC01239 | PAPPA | RNF180 | ZBTB20 |
| CADPS | GLIS1 | LINC01614 | PAPPA2 | ROR1 | ZC3H12B |
| CAMK2D | GPC4 | LNX2 | PARD3 | SDK1 | ZHX2 |
| CARMIL1 | GRIN2A | MAML3 | PLCB4 | SGCD | ZMAT1 |
| CILP | HMCN1 | MAN1C1 | PLXDC2 | SLC12A2 | |
| COL14A1 | HMGA2 | MAP7D3 | PLXNA4 | SRGAP1 | |
| DACH2 | IRAG1 | MAPK10 | PRG4 | THSD7A | |
| DIAPH2 | ITGA11 | MDC1 | PRKG1 | TMEM117 | |

Expression of gene sets of PUFIN3 TEP was validated using the data available in public bulk RNA-seq database. FIGS. 21E and 21F show that expression of gene sets of PUFIN3 TEP correlated with human IPF signature (genes listed in Table 18) based on analysis of RNA-seq data reported by Schafer et al., Nat Commun. (2017) (FIG. 21E) and Luzina et al., Cell Immunol. (2018) (FIG. 21F). These results suggest that genes of PUFIN3 TEP were highly expressed in IPF patients and that targeting PUFIN3 provided potential approach to modulate disease or revert disease transcription.

Results of bulk RNA-seq heatmap of the top 100 most downregulated genes based on PUFIN3 L1 ASO dose response showed that these downregulated genes displayed dose-responsive decrease associated with PUFIN3 L1 ASO treatment (data not shown). Construction of protein-protein interaction network of genes in PUFIN3 TEP was performed as described in the previous example. PUFIN3 TEP protein-protein functional interaction network was generated using STRING Database analysis, and genes that were shown to interact with each other were shown in Table 41. Network analysis of PUFIN3 TEP was performed and several pathways such as actin and cell adhesion, Wnt-signaling pathway, and cell assembly, were identified. Actin and cell adhesion pathways were among the largest group of downregulated GSEA pathways, and PUFIN3 might be involved in reactivating dormant wound healing pathway via Wnt-signaling. This network showed that genes in PUFIN3 TEP formed a network that might be heavily regulated by ADAM12 and might be involved with Wnt signaling pathways.

TABLE 41
Genes in PUFIN 3 TEP that were predicted to have protein-protein
functional interaction.

| | | | | | |
|---|---|---|---|---|---|
| ADAM12 | CILP | PLCB4 | SLC12A2 | GPC4 | UNC5C |
| SGCD | PRG4 | MYH11 | CADPS | FZD6 | MTSS1 |
| PAPPA | PRKG1 | IRAG1 | KIF26B | ROR1 | RNF180 |
| PAPPA2 | CACNA1C | PLXDC2 | PLXNA4 | CADPS | SRGAP1 |
| ITGA11 | PARD3 | CAMK2D | THSD7A | SLC12A2 | |
| FNDC1 | DIAPH2 | GRIN2A | BTBD9 | OPCML | |
| COL14A1 | DACH2 | OPCML | PTPRD | GRIN2A | |

Transcription factor enrichment analysis in genes of PUFIN3 TEP was performed as described in the previous example, and results show that STAT3, WT1, SMAD4, SMARCD1, YAP1, AR, SOX2, NFκB1, PAX3-FKHR, and CEBPD, were among the top transcription factors associated with 74 genes in PUFIN3 TEP. ENRICHR analysis of top Reactome Gene Ontology Pathways associated with genes in PUFIN3 TEP was performed, and results show that top pathways were cGMP effects; synaptic adhesion-like molecules; unblocking of NMDA receptors, glutamate binding and activation; negative regulation of NMDA receptor-mediated neuronal transmission; nitric oxide stimulates guanylate cyclase; long-term potentiation; assembly and cell surface presentation of NMDA receptors; phase 0—rapid depolarization; PLC beta mediated events; G-protein mediated events. Gene Ontology (GO) Pathway Analysis was performed based on GO Molecular Processes, GO Cellular Components, and GO Biological Processes, revealing variety of cellular, molecular, and biological pathways associated with genes in PUFIN3 TEP. Top pathways in GO molecular processes comprised positive regulation of cell projection organization; enzyme-linked receptor protein signaling pathway; muscle organ development; regulation of cardiac muscle contraction by regulation of the release of sequestered calcium ion; cardiac muscle cell; regulation of cardiac muscle contraction by calcium ion signaling; regulation of release of sequestered calcium ion into cytosol by sarcoplasmic reticulum; calcium ion transmembrane import into cytosol; cell-cell adhesion via plasma-membrane adhesion molecules; regulation of cardiac muscle cell contraction. Top pathways in GO cellular components comprised sarcoglycan complex; macropinosome; dystroglycan complex; pinosome; NMDA selective glutamate receptor complex; platelet dense tubular network membrane; glutamatergic synapse; platelet dense tubular network; collagen-containing extracellular matrix; muscle myosin complex. Top pathways in GO biological processes comprised Wnt receptor activity; metalloendopeptidase activity; inorganic cation transmembrane transporter activity; phosphatidylinositol-3-phosphate binding; metallopeptidase activity; alkaline phosphatase activity; ciliary neurotrophic factor receptor binding; coreceptor activity involved in Wnt signaling pathway, plana cell polarity pathway; cell-matrix adhesion mediator activity; mannosyl-oligosaccharide 1,2-alpha-mannosidase activity. ENRICHR analysis of top Wikipathways associated with genes in PUFIN3 TEP was performed, and result is shown in Table 42 below. Wnt signaling pathway was shown to be the top pathway associated with genes in PUFIN3 TEP.

TABLE 42
Pathways associated with genes from PUFIN3 TEP.

| | | | Ad- | | Com- |
|---|---|---|---|---|---|
| In- | | | justed | Odds | bined |
| dex | Name | P-value | p-value | Ratio | score |
| 1 | Wnt Signaling WP428 | 0.00006486 | 0.00733 | 13.17 | 127.04 |
| 2 | Arrhythmogenic Right | 0.002611 | 0.0666 | 11.82 | 70.28 |
| | ventricular cardiomyo- | | | | |
| | pathy WP2118 | | | | |
| 3 | Alzheimer 39 S disease | 0.002774 | 0.0666 | 5.59 | 32.91 |
| | and miRNA effects | | | | |
| | WP2059 | | | | |
| 4 | Alzheimer 39 S disease | 0.002774 | 0.0666 | 5.59 | 32.91 |
| | WP5124 | | | | |
| 5 | Regulatory circuits of | 0.003032 | 0.0666 | 11.18 | 64.85 |
| | the STAT3 signaling | | | | |
| | pathway WP4528 | | | | |
| 6 | Physiological and | 0.003536 | 0.0666 | 25.13 | 141.96 |
| | pathological | | | | |
| | hypertrophy of the | | | | |
| | heart WP1528 | | | | |
| 7 | Disruption of post- | 0.006621 | 0.9843 | 17.83 | 89.45 |
| | synaptic signaling | | | | |
| | by CNV SP4875 | | | | |
| 8 | Vitamin D sensitive | 0.007844 | 0.9843 | 16.25 | 78.79 |
| | calcium signaling | | | | |
| | in depression | | | | |
| | WP4698 | | | | |
| 9 | Wnt signaling in | 0.007844 | 0.9843 | 16.25 | 78.79 |
| | kidney disease | | | | |
| | WP4150 | | | | |
| 10 | Sleep regulation | 0.008711 | 0.9843 | 15.35 | 72.79 |
| | WP3591 | | | | |

As shown in FIG. 21G, which was analyzed from public bulk RNAseq data (Ahangari et al., JCI Insight. (2023)), expression of PUFIN3 was predicted to decrease in an ex vivo human lung tissue model after post-fibrotic treatment, e.g., PNA33. Expression of genes in PUFIN3 TEP and expression of genes in IPF gene signatures were also predicted to decrease in this dataset. This result shows that targeting PUFIN3 can have anti-fibrotic effects.

Based on analysis of scRNAseq results of ex vivo model reported by Lang et al., Sci Tran Med. (2024), genes in PUFIN3 TEP and in IPF gene signature showed highest expression in mesenchymal/myofibroblast cell types (data not shown). In this report, three ex vivo conditions were tested: control cocktail (CC), fibrotic cocktail (FC), and fibrotic cocktail+Nintedanib (FC+Nintedanib). FIG. 21H shows that expression of genes in PUFIN3 TEP was increased with FC and was decreased after Nintedanib treatment, suggesting that genes in PUFIN3 TEP can be used to predict anti-fibrotic effects and/or efficacy of a drug for treating fibrosis.

Overall, PUFIN3 TEP can be used to investigate PUFIN1 mechanism of action (MoA) or to access or compare ASO potency and efficacy.

### Example 5: ASO Knockdown of PUFIN2

In this example, ASO sequences were developed in silico to target PUFIN2, as identified in the Example 1.

Methods of ASO design and generation as well as experiments are performed as described in the Example 2.

Antisense oligonucleotides (ASOs) comprising locked nucleic acid (LNA) modifications are designed and generated. Table 9 shows the ASO sequences generated in silico to target PUFIN2.

Gene expression of PUFIN2 lncRNA and canonical fibrotic gene expression markers are examined by using qPCR or bulk RNAseq.

### Results:

In a merged module network analysis, PUFIN2 was co-expressed with protein coding genes and may have similar functions to cilium genes in these pathways (data not shown). Genes associated with PUFIN2 co-expression network were, for examples, CCDC60, FAM81B, TCTE1, MDH1B, RSPH9, PACRG, C9orf116, ODAD4, TMEM231, and CFAP300.

### Additional Screening

Further, in this example, primary human lung fibroblasts (pHLFs) were utilized to screen ASOs targeting PUFIN2. A total of 13 ASOs, which were 12 LNAs and 1 control (scramble, non-target control) ASO, were screened. Biological replicates were n=3. Student t-test was performed for statistical analysis of each ASO compared to scramble.

The ASO sequences generated in silico to target PUFIN2 can be found in Table 9. Table 22 shows the ASO sequences designed and generated to target PUFIN2, and these sequences were tested in this example.

TABLE 22
Sequences of ASOs tested in this example to
target PUFIN2.

| | SEQ | | |
|---|---|---|---|
| Figure | ID | Sequence | Modifi- |
| Legends | NO: | (from 5′ to 3′) | cation |
| Scr/Scramble | 2567 | AACACGTCTATACGC | LNA |
| L1 | 1 | GTAATGCCTAGCAGTG | LNA |
| L2 | 2 | CGAATCCAGACCCAAG | LNA |
| L3 | 3 | GGCATACTCAATAGCT | LNA |
| L4 | 4 | CAAGCTGAATAGGACC | LNA |
| L5 | 5 | CTTAAGGGTGCTGGTC | LNA |
| L6 | 6 | GCATCTGTTGTGAGTC | LNA |
| L7 | 7 | CTATCTAGTGAACACC | LNA |
| L8 | 8 | GGGTCGAAGTCTGAAC | LNA |
| L9 | 9 | GTAGTCCTGGTTTAGA | LNA |
| L10 | 2564 | GCCACAACCTACTTAT | LNA |
| L11 | 2565 | GACACATACTACTGGT | LNA |
| L12 | 2566 | CTACTAAGTGTCATCC | LNA |

Notes:
The L1, L2, L3, L4, L5, L6, L7, L8, L9, L10, L11, and L12 sequences described in this table comprise 3 LNA modified nucleotides at the 5′ end and 2 LNA modified nucleotides at the 3′ end, forming 3-11-2 configuration of LNA-ASO GapmeR. Phosphorothioate linkages were incorporated between each of the nucleotides. Scramble ASO comprises 3 LNA modified nucleotides at the 5′ end and 2 LNA modified nucleotides at the 3′ end, forming 3-10-2 configuration of LNA-ASO GapmeR.

Cell Culture, RNA Extraction, and qPCR were Performed as Described in the Previous Examples.

### Results

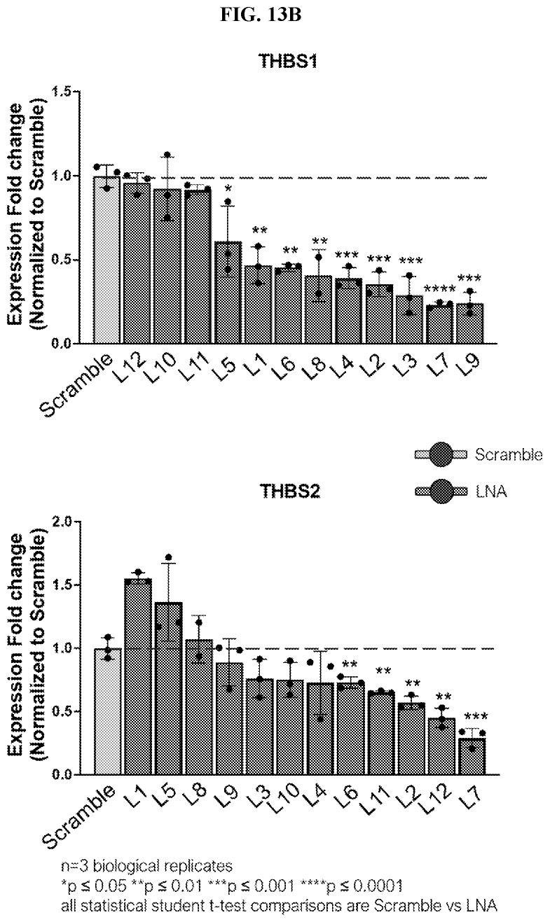

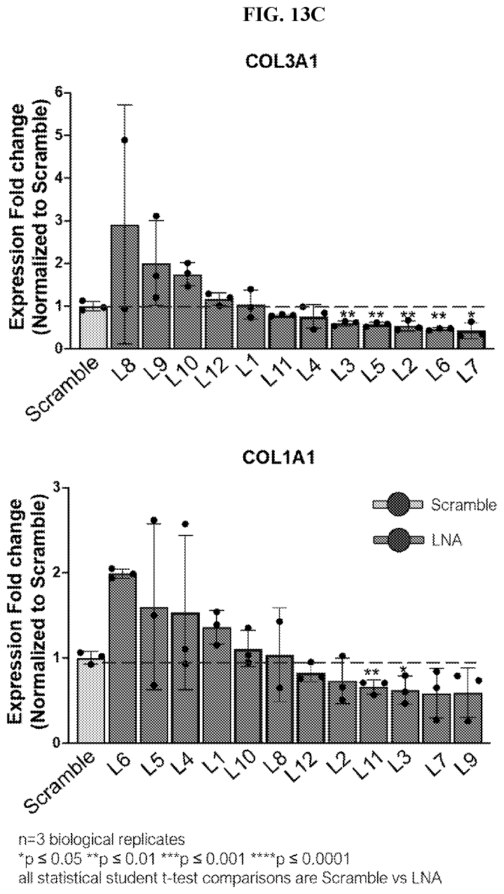

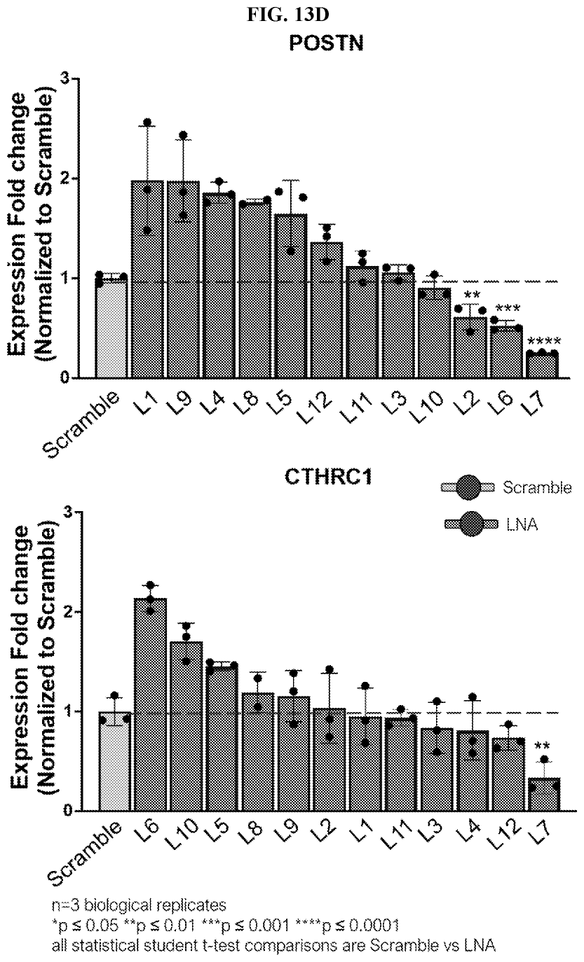

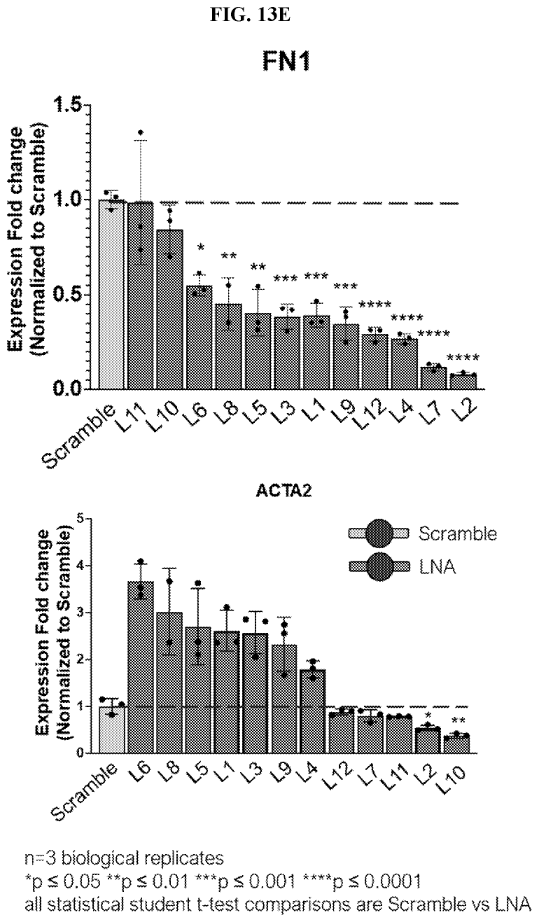

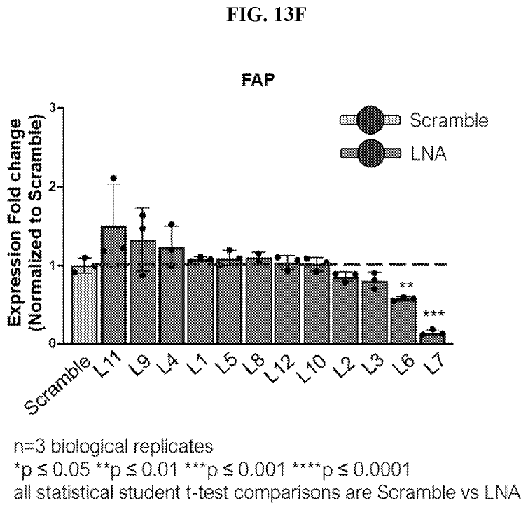

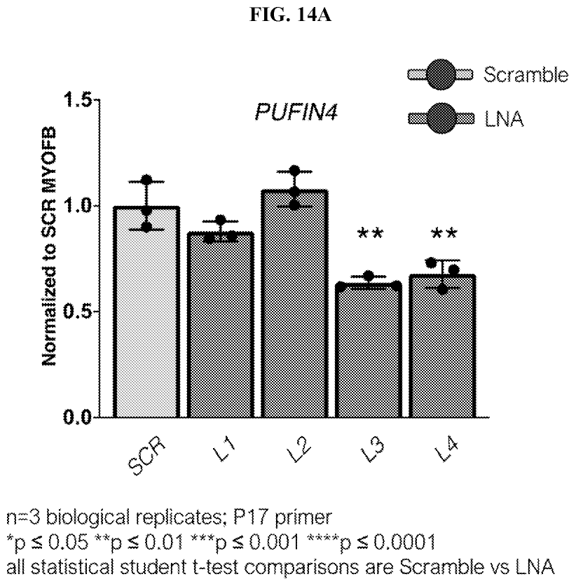

ASOs that were tested in this example were listed in Table 22. As shown in FIG. 13A, expression of PUFIN2 in cells treated with ASOs was significantly downregulated. Expression of PUFIN2 by LNA4 (or L4) (SEQ ID NO: 4) was decreased using primers listed in Table 35 for quantification. FIG. 13B top graph shows that the expression of THBS1 in cells treated with ASOs (SEQ ID NOs: 5, 1, 6, 8, 4, 2, 3, 7, 9) was significantly downregulated compared to cells treated with scramble ASO. THBS1 is the closest coding gene located near genomic loci encoding PUFIN2. FIG. 13B bottom graph shows that THBS2 expression in cells treated with ASOs (SEQ ID NOs: 6, 2565, 2, 2566, 7) was significantly downregulated. FIG. 13C bottom graph shows that COL1A1 expression in cells treated with ASOs (SEQ ID NOs: 2565, 3) was significantly downregulated. FIG. 13C top graph shows that COL3A1 expression in cells treated with ASOs (SEQ ID NOs: 3, 5, 2, 6, 7) was significantly decreased. FIG. 13D top graph shows that POSTN expression in cells treated with ASOs (SEQ ID Nos: 2, 6, 7) was downregulated. FIG. 13D bottom graph shows that CTHRC1 expression in cells treated with ASOs (SEQ ID NO: 7) was significantly downregulated. FIG. 13E top graph shows that FN1 expression in cells treated with ASOs (SEQ ID NOs: 6, 8, 5, 1, 9, 2566, 4, 7, 2) was downregulated. FIG. 13E bottom graph shows that ACTA2 expression in cells treated with ASOs (SEQ ID NOs: 2, 2564) was downregulated. FIG. 13F shows that FAP expression in cells treated with ASOs (SEQ ID NOs: 6, 7) was downregulated. These results were determined by evaluating the relative expression of the genes via qRT-PCR as described in the previous example.

### Example 6: ASO Knockdown of PUFIN6

In this example, primary human lung fibroblasts (pHLFs) were utilized to screen ASOs targeting PUFIN6. A total of 14 ASOs, which were 10 LNA-modified ASOs, 3 MOE-modified ASOs, and 1 control (scramble, non-target control) ASO, were screened. Biological replicates were n=3. Student t-test was performed for statistical analysis of each ASO compared to scramble.

### Methods

### ASO Designs and Generation

ASOs were designed and generated as described in Example 2. The ASO sequences generated in silico to target PUFIN6 can be found in Table 10. Table 21 shows the ASO sequences designed and generated to target PUFIN6, and these sequences were tested in this example.

TABLE 21
Sequences of ASOs tested in this example to target PUFIN6.

| Figure | SEQ | Sequence | |
|---|---|---|---|
| Legends | ID NO: | (from 5′ to 3′) | Modification |
| Scr/Scramble | 2567 | AACACGTCTATACGC | LNA |
| L1 | 17 | TTATCATCAGTCTAGG | LNA |
| L2 | 18 | CTCCGACTGTGCAATG | LNA |
| L3 | 20 | ATGACAGACCTCGTGT | LNA |
| L4 | 21 | GAATGTAGCTCCTTAG | LNA |
| L5 | 22 | GGATCAACTACAGGGC | LNA |
| L6 | 24 | AGACTCTAAAGCTCCG | LNA |
| L7 | 25 | CAAACCCGCCATCCAC | LNA |
| L8 | 19 | GCAGCATATAACTCTC | LNA |
| L9 | 23 | CCACTGAGTATAACTC | LNA |
| L10 | 26 | GTCTGATCCTAGCACC | LNA |
| M1 | 378 | GCTACAATGACCTCAGAGCC | MOE |
| M2 | 385 | CTAGATCTTACAAAGGACCC | MOE |
| M3 | 387 | CTTGGGAAGGTAGAATCTGG | MOE |

Notes:
The L1, L2, L3, L4, L5, L6, L7, L8, L9, and L10 sequences described in this table comprise 3 LNA modified nucleotides at the 5′ end and 2 LNA modified nucleotides at the 3′ end, forming 3-11-2 configuration of LNA-ASO GapmeR. Phosphorothioate linkages were incorporated between each of the nucleotides. The M1, M2, and M3 sequences described in this table comprise five 2′-MOE modified nucleotides at the 5′ end and five 2′-MOE modified nucleotides at the 3′ end, forming a 5-10-5 configuration of 2′-MOE-ASO GapmeR. Scramble ASO comprises 3 LNA modified nucleotides at the 5′ end and 2 LNA modified nucleotides at the 3′ end, forming 3-10-2 configuration of LNA-ASO GapmeR.

### Experiment was Performed as Described in Example 2.

### Results

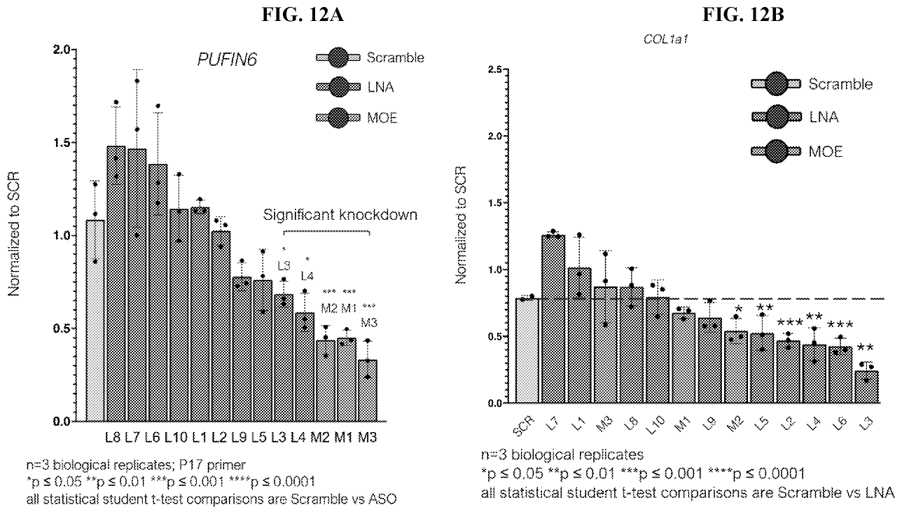

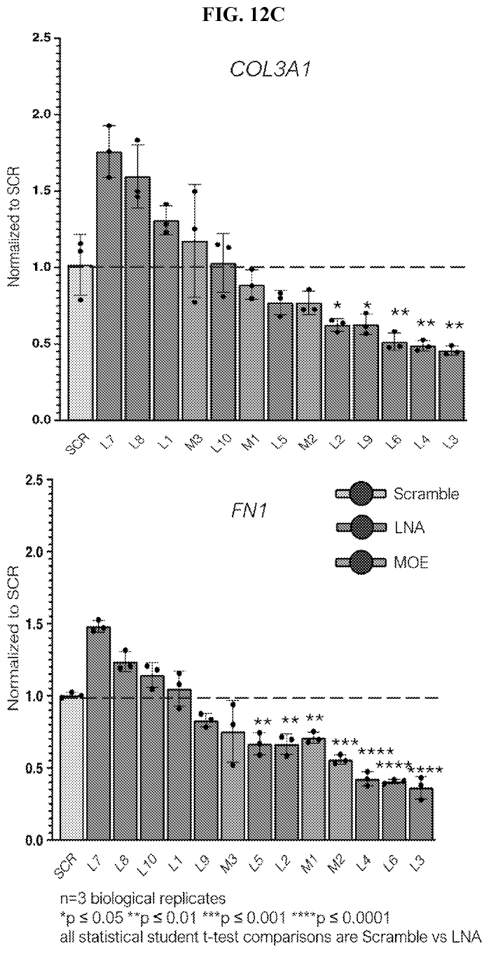

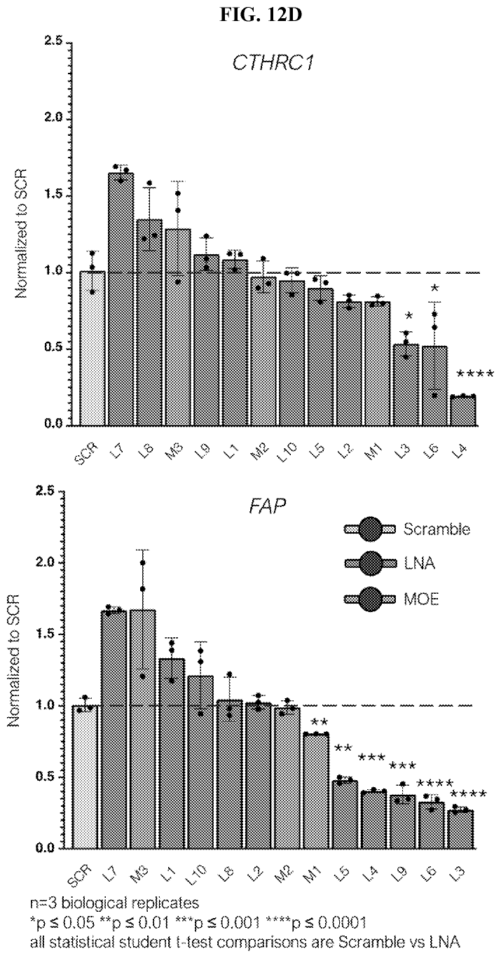

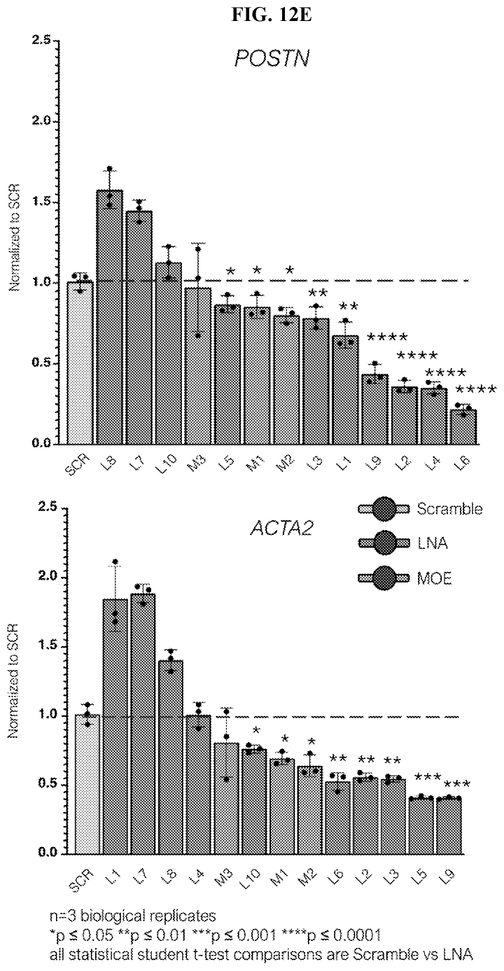

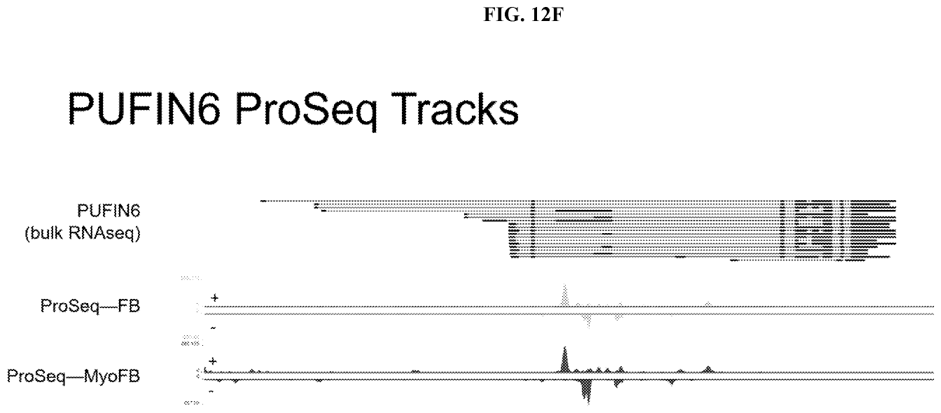

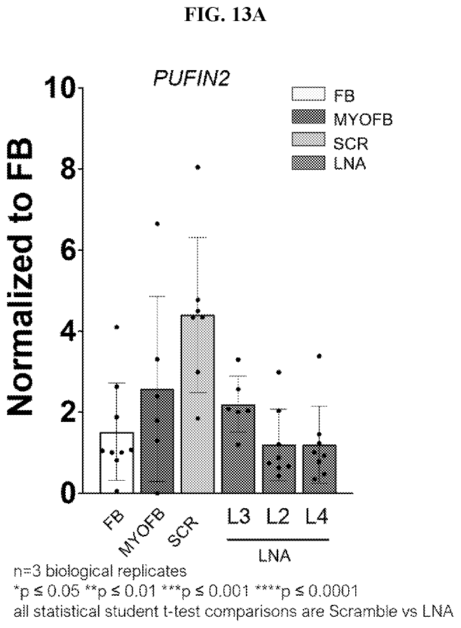

ASOs tested to target PUFIN6 were listed in Table 21. As shown in FIG. 12A, expression of PUFIN6 in cells treated with ASOs (SEQ ID NOs: 20, 21, 378, 385, 387) in Table 21 was significantly downregulated. MOEs, which were M1, M2, and M3, listed in Table 21, were designed to target a conserved exon, which was mapped in all potential isoforms of PUFIN6 based on bulk RNAseq and had a strong PROseq signal as shown in FIG. 12F. LNA10 (or L10) (SEQ ID NO: 26) was designed to target to the conserved exon but it didn't show downregulation of the PUFIN6. FIGS. 12B-12E show that expression of fibrotic genes in cells treated with ASOs was significantly downregulated. FIG. 12B shows that COL1A1 expression in cells treated with ASOs (SEQ ID NOs: 385, 22, 18, 21, 24, 20) was significantly downregulated. FIG. 12C top graph shows that COL3A1 expression in cells treated with ASOs (SEQ ID NOs: 18, 23, 24, 21, 20) was significantly downregulated. FIG. 12C bottom graph shows that FN1 expression in cells treated with ASOs (SEQ ID NOs: 22, 18, 378, 385, 21, 24, 20) was downregulated. FIG. 12D top graph shows that CTHRC1 expression in cells treated with ASOs (SEQ ID NOs: 20, 21,24) was significantly downregulated. FIG. 12D bottom graph shows that FAP expression in cells treated with ASOs (SEQ ID NOs: 378, 22, 21, 23, 24, 20) was significantly downregulated. FIG. 12E top graph shows that POSTN expression in cells treated with ASOs (SEQ ID NOs: 23, 18, 21, 24) was significantly downregulated. FIG. 12E bottom graph shows that ACTA2 expression in cells treated with ASOs (SEQ ID NOs: 26, 378, 385, 24, 18, 20, 22, 23) was significantly downregulated. These results were determined by evaluating the relative expression of the genes via qRT-PCR as described above.

### Example 7: ASO Knockdown of Additional PUFIN

In this example, ASO sequences are developed in silico to target the lncRNAs (PUFIN) such as PUFIN5, PUFIN9, PUFIN10, PUFIN11, and PUFIN12, as identified in the Example 1.

Methods of ASO design and generation as well as experiments are performed as described in the Example 2.

Antisense oligonucleotides (ASOs) comprising locked nucleic acid (LNA) modifications are designed and generated. Tables 13-16 show the ASO sequences generated in silico to target additional PUFINs (PUFIN5, PUFIN9, PUFIN10, PUFIN11, and PUFIN12).

Gene expression of PUFIN5, PUFIN9, PUFIN10, PUFIN11, and PUFIN12 and canonical gene expression markers of fibrosis are investigated by using qPCR or bulk RNAseq.

### Example 8: Ex Vivo Fibrotic Treatment Induced Fibrotic Phenotypes in Human Precision Cut Lung Slices (hPCLS)

A study was designed to test the induced fibrotic model on respiratory tissue using human precision-cut lung slices (hPCLS). The ex vivo PCLS model has employed in the study of IPF, a lung disease characterized by excessive extracellular matrix deposition and abnormal tissue remodeling. The PCLS model offers an ex vivo platform that replicates the structural and functional characteristics of the lung tissue, allowing investigation of aspects of IPF pathogenesis and assessment of the efficacy of potential therapeutic interventions in human tissues. The PCLS model enables the evaluation of various candidate drugs and therapies in a physiologically relevant context, offering insights into their potential efficacy and safety before progressing to in vivo studies. The ability of PCLS model to maintain the human lung's structural complexity and functionality makes it a valuable model for dissecting the molecular and cellular mechanisms underlying IPF, exploring potential therapeutic targets, and accelerating the development of novel treatments.

In this example, fibrosis-like changes were induced in healthy ex vivo hPCLS. The hPCLS were exposed to a fibrotic cocktail (FC) at select time points including at day 4 and day 8 post-exposure. The effects on the tissues were determined by measuring protein marker release, e.g., cytokine release using immunoassay, and expression analysis, e.g., RNA Sequencing.

### Methods

Cryopreserved hPCLS (~8 mm in diameter, with a 500±100 μm mean thickness, cultured submerged) from lung tissue of healthy individual were used in this example. Briefly, cryopreserved ex vivo hPCLS were thawed and cultured. Three conditions were utilized: 1) control cocktail (CC) (negative control) 2) fibrotic cocktail (FC) and 3) Lipopolysaccharide (LPS) treatment (positive control). Five hPCLS were used for the FC and the negative control groups, and three hPCLS were used for the positive control group.

Negative control(s): control cocktail (CC).

Positive control(s): Lipopolysaccharide (LPS) (5 μg/mL). LPS can be used as a positive control for fibrotic condition. LPS dosage is a model of acute exacerbation IPF. LPS is typically introduced to mice via intratracheal or intranasal routes. Upon exposure to LPS, a cascade of inflammatory and immune responses is triggered. These responses encompass the recruitment and activation of various immune cells, the release of pro-inflammatory cytokines, and the generation of reactive oxygen species (ROS). This initial inflammatory phase ultimately contributes to lung injury by disrupting the lung's delicate alveolar structure and function. As the inflammation persists, it can lead to the development of IPF.

Fibrotic cocktail (FC); recombinant transforming growth factor-β (TGF-β) (5 ng/ml), platelet-derived growth factor-AB (PDGF-AB) (10 ng/mL), tumor necrosis factor (TNF-α) (10 ng/mL), and lysophosphatidic acid (LPA) (5 μM).

Cryopreserved hPCLS obtained from lung tissue of healthy individual were thawed and placed in 24-well plates (1 slice/well), with 1 mL of culture media per well and cultured under submerged conditions during the acclimation period (2 to 4 days) at standard culture conditions (37±1° C. in a humidified (90±10%) incubator containing 5±1% CO₂). The culture media was replaced every 2 to 3 days thereafter. Following the acclimation period, the hPCLS were used for treatment initiation on day 0.

For sample and data collection, the tissues and culture media were harvested and collected at day 4 and day 8 post-exposure to the control cocktail (CC), positive control (LPS), or fibrotic cocktails (FC), respectively.

At 4 days post-exposure, for fibrotic cocktail (FC) and the control cocktail (CC) groups only, media and tissues were harvested and stored frozen. Tissues were stored in RNA later for RNA sequencing. The remaining tissues were cultured with fresh material.

At 8 days post-exposure, for all groups, media and tissues were harvested and stored frozen. Tissues were stored in RNA later for RNA sequencing.

Immunoassay: A multiplex assay (Luminex) was used to measures multiple analytes in culture media of the sample. The multiplex assay was run as per the manufacturer's manual. The assay reagents, standards and sample dilutions (if applicable) were prepared per the instructions in the manual. The multiplex analyte panel used in this example comprised: IL-6, IL-8, MCP-1, TIMP-1, fibronectin, pro-collagen Iα1, MMP-1, MMP-7, MMP-3, VEGF, IL-2, and IL-7. Luminex's xPONENT software was used to process data sets. The analyte concentrations obtained from xPONENT software were sorted and presented as mean±standard deviation per group, and/or as fold change relative to negative control group.

cDNA library building and total bulk RNA Sequencing: Total RNA was isolated using the High Pure RNA Isolation Kit according to the manufacturer's instruction. Illumina Ribo-Zero kit (Cat #20040526) was used to build total RNA libraries. Libraries were diluted 1:10 and quality was accessed on the D1000 high sensitivity ship (Agilent TapeStation, Cat #5067-5584). RNA sequencing was performed to determine expression of lncRNAs, e.g., PUFINs, and other markers. Transcriptome analysis of the human tissue slices was performed. Transcriptomic gene expression was accessed via bulk RNAseq for both coding and non-coding genes. For bulk RNAseq, 1 lane of the NovaSeq X was used to sequence the 23 samples. Approximately 38 million reads/sample were aligned. Reads per samples showed roughly equal distributions. GC bias was consistent between samples even though some bias was present. Adaptor content was <2% and no over-represented sequence was found.

### Results

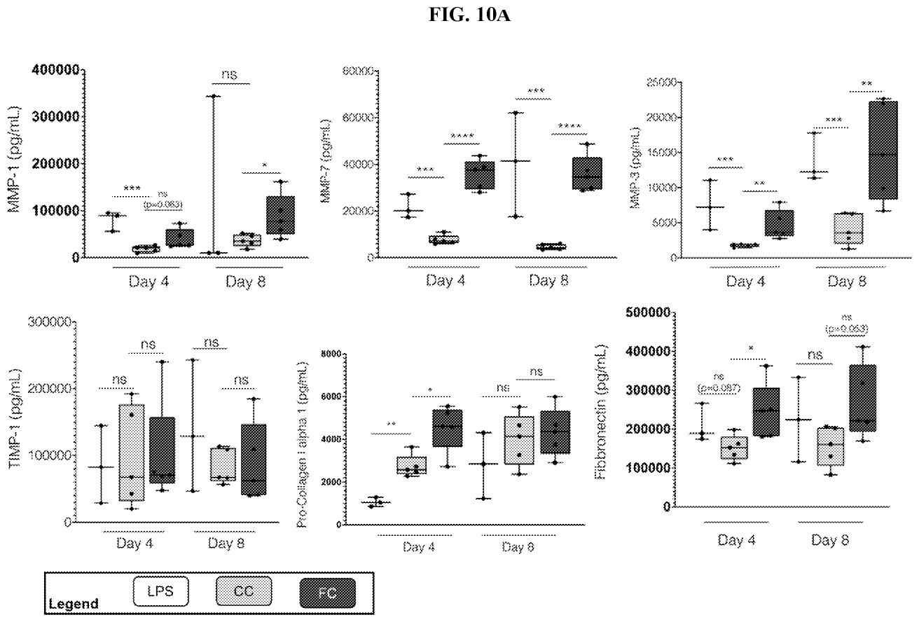

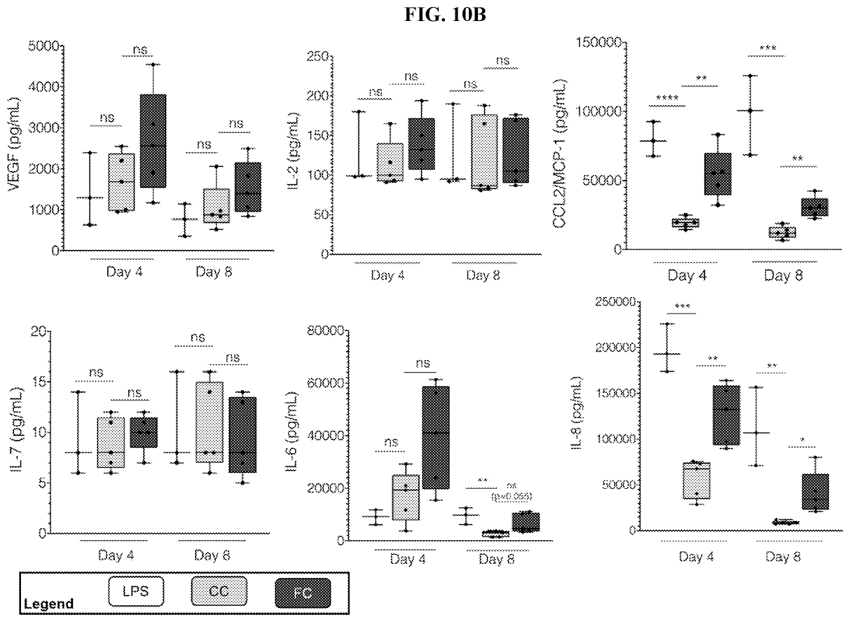

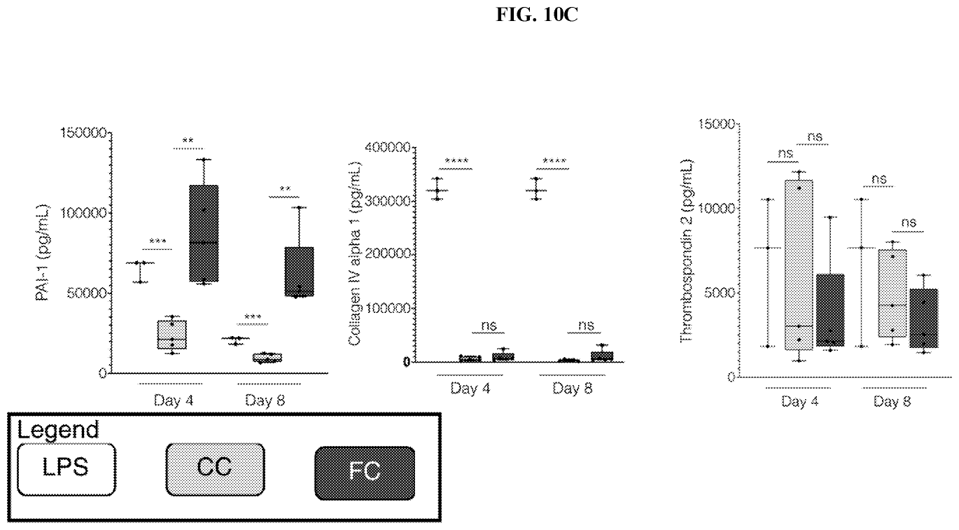

Results from Luminex 12-plex assay are shown in FIGS. 10A-10C. For MMP analytes, the secreted protein level of MMP-7 was significantly higher at day 4 in culture media of FC group compared to culture media of CC group, day 8 in culture media of FC group compared to culture media of CC group, day 4 in culture media of LPS group compared to culture media of CC group, and day 8 in culture media of LPS group compared to culture media of CC group. The secreted protein level of MMP-3 was significantly higher at day 4 in culture media of FC group compared to culture media of CC group, day 8 in culture media of FC group compared to culture media of CC group, day 4 in culture media of LPS group compared to culture media of CC group, and day 8 in culture media of LPS group compared to culture media of CC group. The secreted protein level of MMP-1 was significantly higher at day 4 in culture media of LPS group compared to culture media of CC group, and day 8 in culture media of FC group compared to culture media of CC group (FIG. 10A, top row). There was no significant difference for the secreted protein level of TIMP-1 level in culture media of FC group or culture media of LPS group when compared to culture media of CC group (FIG. 10A, bottom left). For fibrotic analytes, at day 4, the secreted protein level of pro-collagen I alpha I was significantly higher in culture media of FC group compared to culture media of CC group. Secreted protein level of fibronectin was significantly higher in culture media of FC group compared to culture media of CC group at day 4, and higher at day 8 in culture media of FC group compared to culture media of CC group although not significant (FIG. 10A, bottom center and bottom right). There were no significant differences for the secreted protein level of VEGF in culture media among different groups (FIG. 10B, top left). For cytokine analytes, at day 8, the secreted protein level of IL-6 was significantly higher in culture media of LPS group compared to culture media of CC group (FIG. 10B, bottom center). The secreted protein level of IL-8 was significantly higher at day 4 in culture media of FC group compared to culture media of CC group, day 8 in culture media of FC group compared to culture media of CC group, day 4 in culture media of LPS group compared to culture media of CC group, and day 8 culture media of LPS group compared to culture media of CC group (FIG. 10B, bottom right). There was no significant difference in the secreted protein levels of IL-2 and IL-7 in culture media among LPS group, CC group, or FC group (FIG. 10B, top center and bottom left, respectively). The secreted protein level of CCL2/MCP-1 was significantly higher at day 4 in culture media of FC group compared to culture media of CC group, day 8 in culture media of FC group compared to culture media of CC group, day 4 in culture media of LPS group compared to culture media of CC group, and day 8 in culture media of LPS group compared to culture media of CC group (FIG. 10B, top right). Additional analytes, e.g., PAI-1, Collagen IV alpha 1, and Thrombospondin 2, were analyzed. The secreted protein level of PAI-1 is significantly upregulated at day 4 in culture media of FC group compared to culture media of CC group, day 8 in culture media of FC group compared to culture media of CC group, day 4 in culture media of LPS group compared to culture media of CC group, and day 8 in culture media of LPS group compared to culture media of CC group (FIG. 10C). The secreted protein level of collagen IV alpha 1 was significantly higher in culture media of LPS group compared to culture media of CC group in both day 4 and day 8. There was no significant difference in the secreted protein level of Thrombospondin 2 in culture media among these groups (FIG. 10C). Biological replicates: Day 4: LPS n=3, CC n=5, Fc n=5. Day 8: LPS n=3, CC n=5, FC n=5. ns not significant *p<0.05 **p<0.01 ***p<0.001 ****p<0.00001. Overall, the results of the analyte analysis showed 7 analyte levels that significantly increased in culture media of FC group compared to culture media of CC group (MMP7, MMP3, COL1A1, FN1, CCL2, IL-8, and PAI-1) and 8 analyte levels that significantly increased in culture media of LPS group compared to culture media of CC group (MMP1, MMP7, MMP3, COL1A, CCL2, IL-8, PAI-1, and COL4A1) at day 4. For day 8, there were 5 significant analyte levels that increased in culture media of FC group compared to culture media of CC group (MMP7, MMP3, CCL2, IL-8, and PAI-1), and 8 analyte levels that were significantly increased in culture media of LPS group compared to culture media of CC group (MMP1, MMP7, MMP3, CCL2, IL-6, IL-8, PAI-1, and COL4A1).

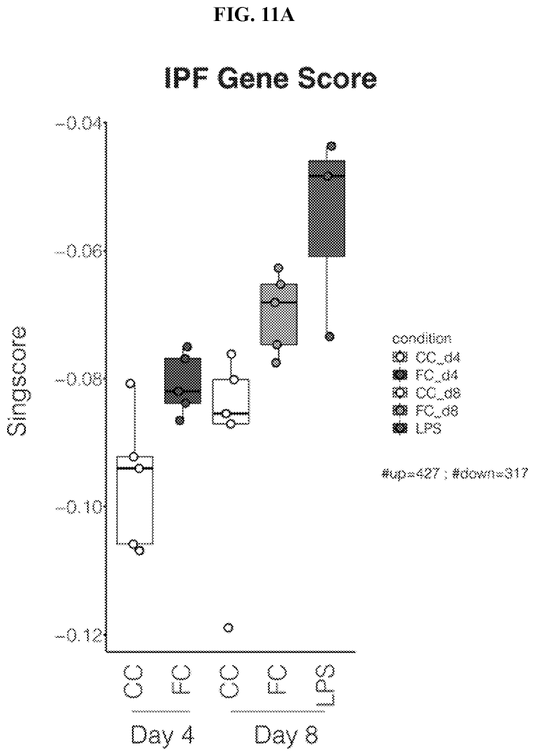

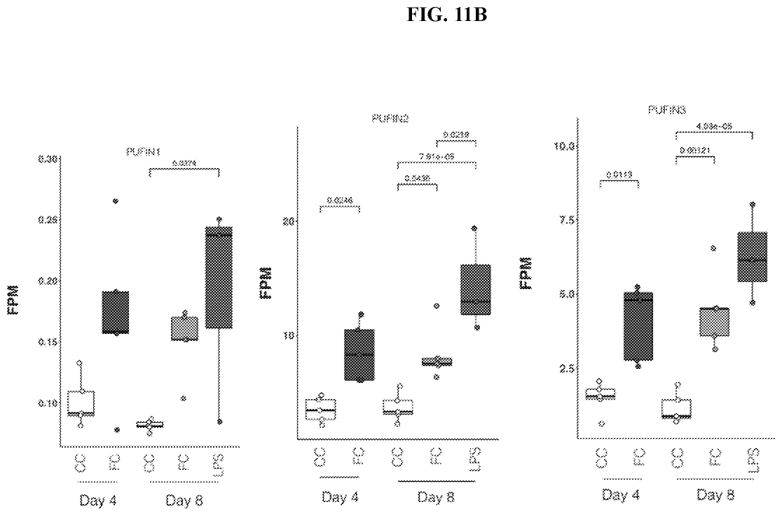

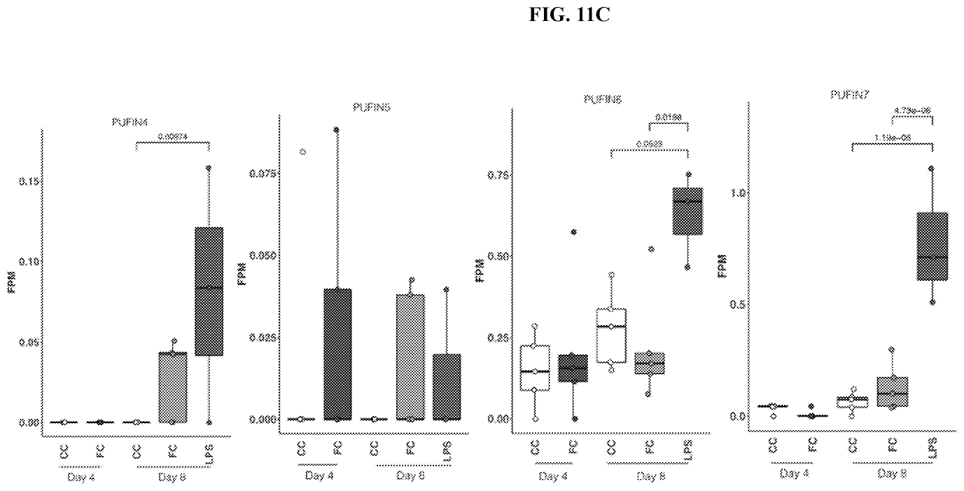

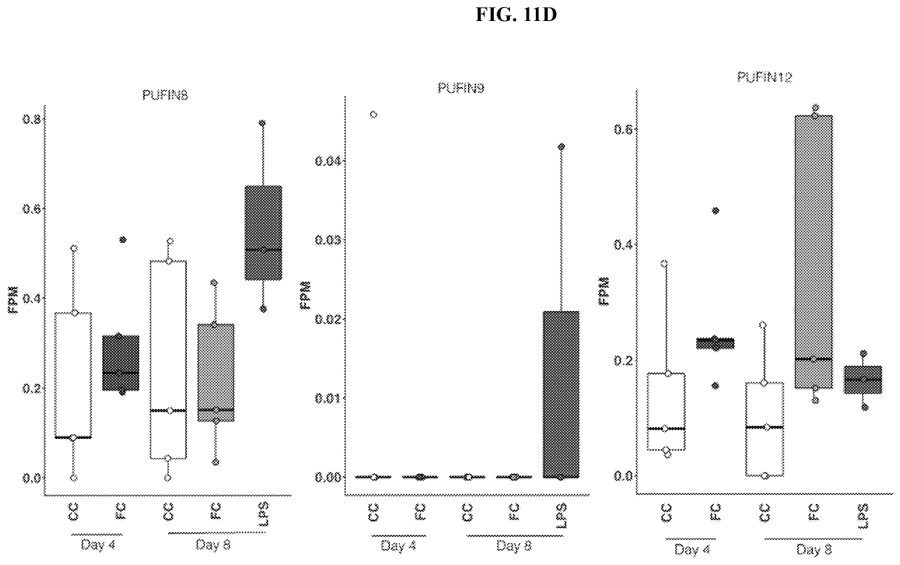

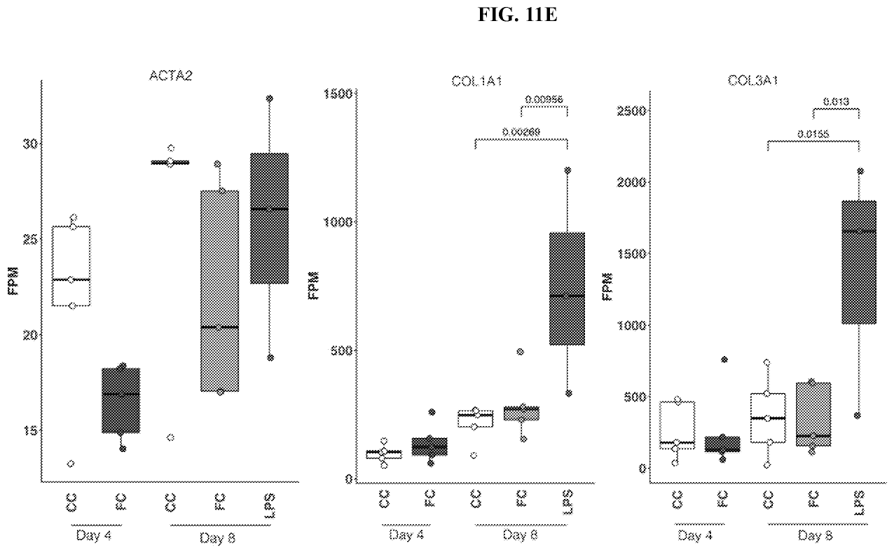

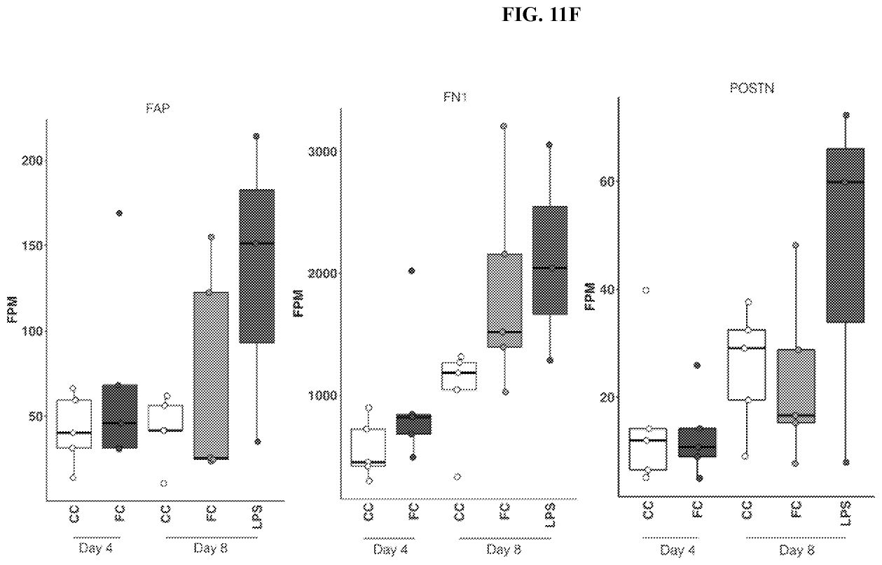

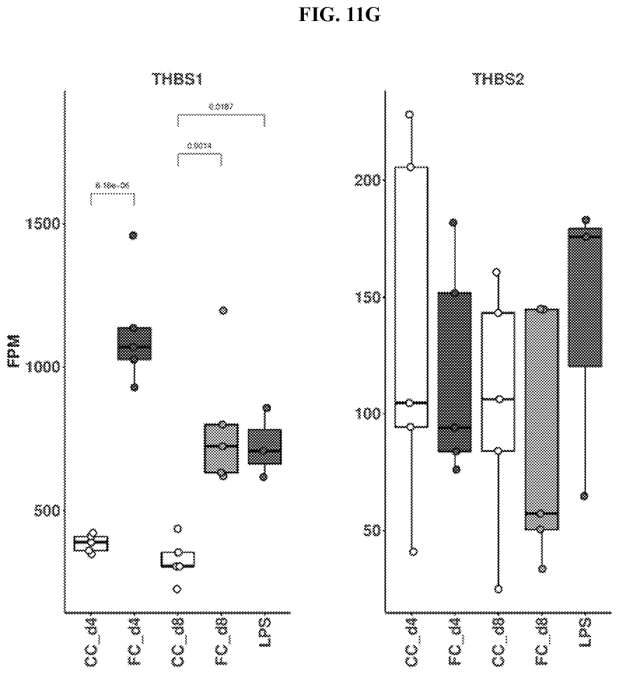

Results from RNA Sequencing are shown in FIGS. 11A-11G. PCA clustering of samples and read QC from bulk RNAseq were performed and results showed that, based on gene expression, samples obtained from CC group were roughly clustered together, and were separated from samples obtained from FC group, which were roughly clustered together (data not shown). As shown in FIG. 11A, the IPF gene score (Singscore) showed that gene expression of FC group was increased at Day 4 and Day 8 compared to gene expression of CC group. Also, IPF gene score (Singscore) increased the most in LPS group compared to FC group and/or CC group. Consistent with the PCA plot, overall expression of IPF gene showed a high level of variation in these samples.

FIGS. 11B-11D show expression of PUFIN in ex vivo hPCLS upon no treatment (CC), FC treatment, or LPS treatment at day 4 and day 8. As shown in FIG. 11B, expression of PUFIN2 and PUFIN3 was increased at day 4 and at day 8 in FC group compared to expression level in CC group. Expression of PUFIN1, PUFIN6, and PUFIN7 in LPS group was increased at day 8 compared to expression in CC group, which are shown in FIGS. 11B-11C. These results showed that expression of PUFIN1, PUFIN3, and PUFIN 7, were significantly induced in fibrotic (LPS) condition using ex vivo hPCLS.

FIGS. 11E-11G show results of expression of fibrotic protein coding gene. As shown in FIG. 11G, expression of THBS1 was significantly upregulated in the FC group compared to expression in CC group at day 4 and day 8 respective. At day 8, expressions of COL1A1, COL3A1, and THBS1 were significantly upregulated in LPS group compared to expression in CC group, which are shown in FIGS. 11E and 11G.

Differential gene expression profiles were analysed. Results of Day 4 comparing expression of FC group and CC group showed that differential expression from samples in the same group were broadly clustered together in this genome-wide gene expression profile (data not shown). With log-fold change >0.5 or <−0.05 and p-adjusted <0.05, there are 2287 up-regulated genes and 3264 down-regulated genes. Similarly, results of Day 8 comparing expression of FC group and CC group showed that differential expression from samples in the same group were broadly clustered together in this genome-wide gene expression profile (data not shown). There were 3374 up-regulated genes and 4018 down-regulated genes in the FC group compared to CC group (log-fold change >0.5 or <−0.05 and p-adjusted <0.05). Results of Day 8 comparing expression of LPS group and CC group also showed that differential expression from samples in the same group were broadly clustered together in this genome-wide gene expression profile (data not shown). There were 1281 up-regulated genes and 1407 down-regulated genes in the LPS group compared to CC group (log-fold change >0.5 or <−0.05 and p-adjusted <0.05).

Overall, the results of the bulk RNAseq analysis indicated higher gene expression scoring (Singscore) of IPF genes in LPS group when compared to gene expression scoring of CC group at day 4 and day 8. Both expression of PUFIN2 and PUFIN3 were significantly upregulated in the FC group when compared to expression from CC group on both day 4 and day 8. Expressions of PUFIN1, PUFIN2, PUFIN3, PUFIN4, PUFIN6, and PUFIN7 were upregulated in LPS group in comparison to expression from CC group on day 8. THBS1 was significantly expressed in the FC group when compared to CC group at both day 4 and day 8. Expression levels of COL1A1 and COL3A1 were significantly higher in LPS group when compared to expression from CC group on day 8.

In this example, IPF phenotypes in ex vivo models using hPCLS were induced using fibrotic cocktails (FC). Results show that the fibrotic cocktail and the LPS positive control stimulated the expression of PUFINs. The top PUFIN, e.g., PUFIN1, PUFIN2, PUFIN3, PUFIN6 & PUFIN7, were induced by LPS administration in hPCLS (FIGS. 11B-11C).

These data suggest different mechanisms that induced expression of PUFINs. First, reagents from the fibrotic cocktail (FC) stimulated PUFIN2 and PUFIN3 expression in hPCLS at multiple time points compared to expression in hPCLS in control cocktail (CC) group. Secondly, the acute immune stimulation by LPS caused PUFIN1, PUFIN4, PUFIN6 and PUFIN7 to express (e.g., PUFIN 2 and PUFIN3 also expressed). This suggests that expression of these PUFINs in myofibroblasts might need immune stimulation or immune cell infiltration, which is a characteristic of IPF.

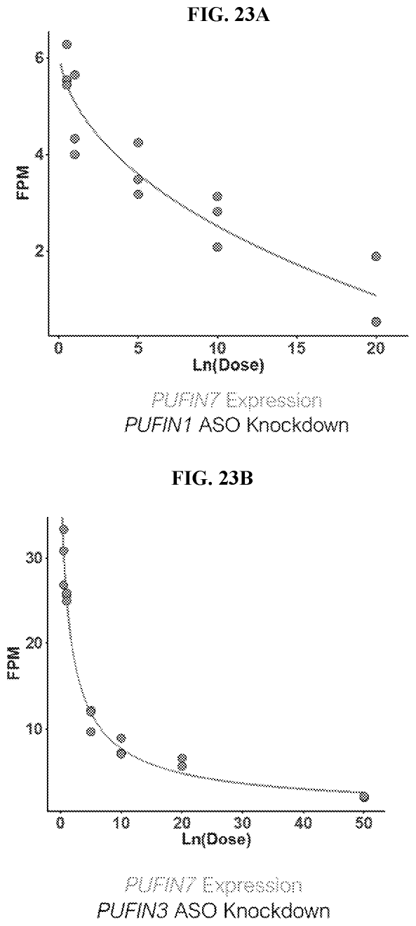

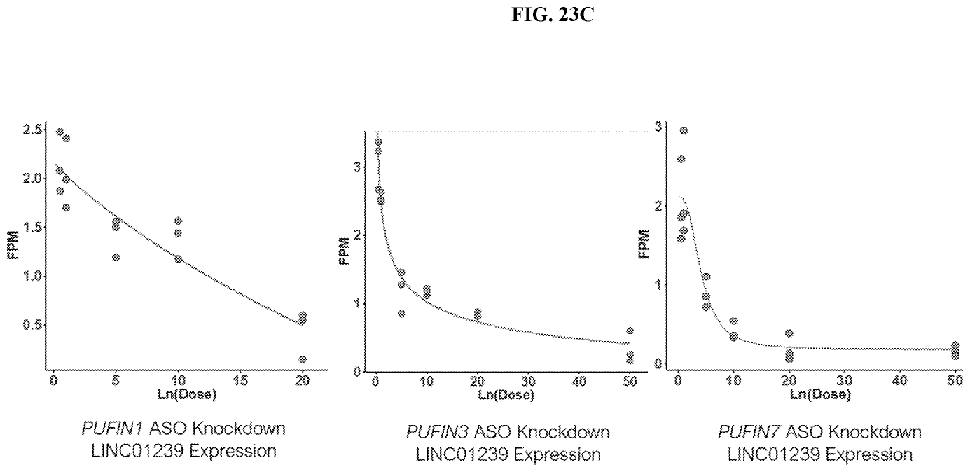

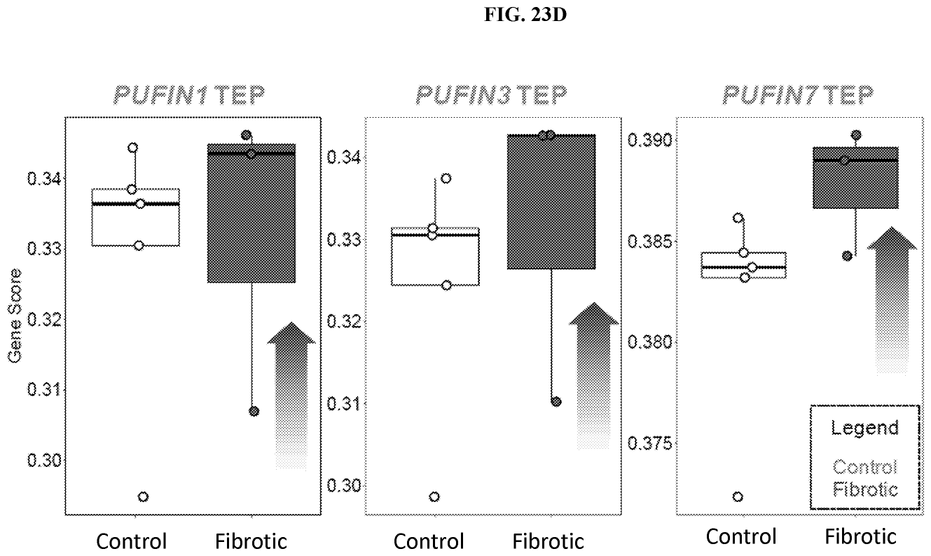

Genes associated with PUFIN1 TEP, PUFIN3 TEP, and PUFIN7 TEP, as described in the previous examples, were analyzed in human ex vivo lung model, and results are shown in FIG. 23D. These genes in TEPs were activated in fibrotic ex vivo hPCLS (LPS positive control), suggesting that ASOs targeting PUFINs have a high likelihood to functionally impact the disease transcriptome.

This example shows detecting pro-fibrotic analytes in culture media and measuring expression of PUFINs using this ex vivo FC model. The analytes and gene expression Singscore indicated that fibrosis broadly occurred. The PUFINs that can be candidates for utilization as biomarkers of the FC model comprised measuring expression of PUFIN2 and PUFIN3. Expressions of PUFIN2, PUFIN3, and their closest protein coding gene THBS1 were all upregulated at day 4 and day 8 in hPCLS treated with the FC condition. This suggests that this region was heavily upregulated in this FC model that was shown to mimic the early stages of IPF injury, which is stimulated by abnormal wound repair from airway epithelial cells and mesenchymal progenitor cells.

### Example 9: ASO Knockdown of PUFIN4 Attenuated Fibrotic Gene Expression

In this example, primary human lung fibroblasts (pHLFs) were utilized to screen ASOs targeting PUFIN4. A total of 5 ASOs, which were 4 LNAs and 1 control (scramble, non-target control) ASO, were screened. Biological replicates were n=3. Student t-test was performed for statistical analysis of each ASO compared to scramble.

### Methods

### ASO Designs and Generation

ASOs were designed and generated as described in previous examples. Table 23 shows the ASO sequences designed and generated to target PUFIN4, and these sequences were tested in this example.

TABLE 23
Sequences of ASOs tested in this example to target
PUFIN4.

| | SEQ | | |
|---|---|---|---|
| Figure | ID | Sequence | Modifi- |
| Legends | NO: | (from 5′ to 3′) | cation |
| Scr/Scramble | 2567 | AACACGTCTATACGC | LNA |
| L1 | 2010 | AGTGCTACCCACAAGG | LNA |
| L2 | 2011 | GGACATGCTATTGATG | LNA |
| L3 | 2012 | TTGATGCTAACAGACC | LNA |
| L4 | 2013 | GGACTACACCTTGACA | LNA |

Notes:
The L1, L2, L3, and L4 sequences described in this table comprise 3 LNA modified nucleotides at the 5′ end and 2 LNA modified nucleotides at the 3′ end, forming 3-11-2 configuration of LNA-ASO GapmeR. Phosphorothioate linkages were incorporated between each of the nucleotides. Scramble ASO comprises 3 LNA modified nucleotides at the 5′ end and 2 LNA modified nucleotides at the 3′ end, forming 3-10-2 configuration of LNA-ASO GapmeR.

Experiment was performed as described in previous examples.

### Results

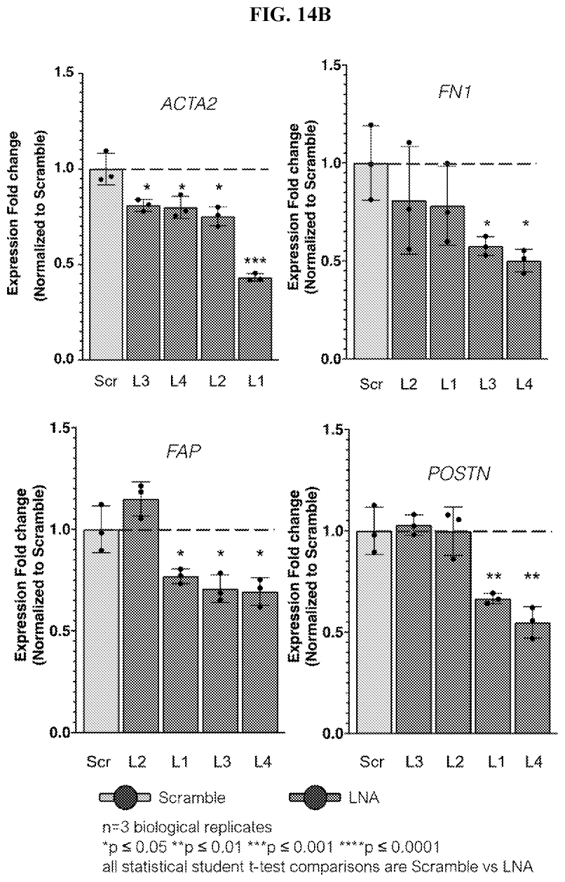

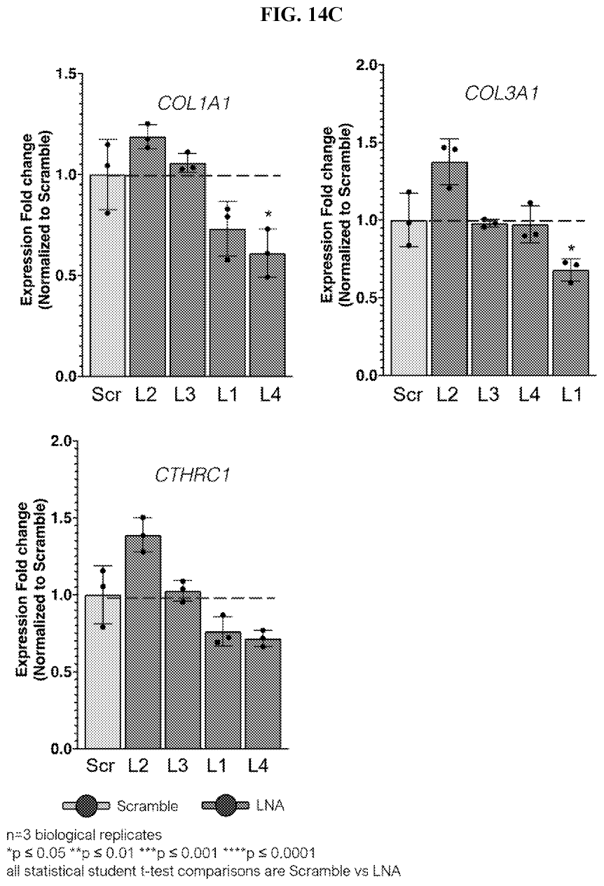

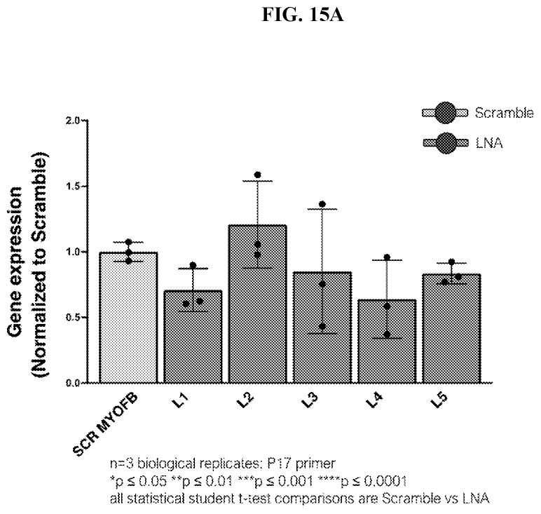

ASOs tested in this example were listed in Table 23. as shown in FIG. 14A, expression of PUFIN4 in cells treated with ASOs (SEQ ID NOs: 2012 and 2013) was significantly downregulated. FIG. 14B shows that ACTA2 expression in cells treated with ASOs (SEQ ID Nos: 2012, 2013, 2011, 2010) was downregulated. FN1 expression in cells treated with ASOs (SEQ ID NOs: 2012, 2013) was downregulated. Expression of FAP in cells treated with ASOs (SEQ ID NOs: 2010, 2012, 2013) was downregulated. Expression of POSTN in cells treated with ASO (SEQ ID NOs: 2010, 2013) was downregulated. FIG. 14C shows that expression of COL1A1 in cells treated with ASOs (SEQ ID NO: 2013) was significantly downregulated. Expression of COL3A1 in cells treated with ASOs (SEQ ID NO: 2010) was significantly downregulated. These results were determined by evaluating the relative expression of the genes via qRT-PCR as described in the previous example. Comparison was performed against Scramble ASO.

### Example 10: ASO Knockdown of PUFIN8 Attenuated Fibrotic Gene Expression

In this example, primary human lung fibroblasts (pHLFs) were utilized to screen ASOs targeting PUFIN8. A total of 6 ASOs, which were 5 LNA-modified ASOs and 1 control (scramble, non-target control) ASO, were screened. Biological replicates were n=3. Student t-test was performed for statistical analysis of each ASO compared to scramble.

### Methods

### ASO Designs and Generation

ASOs were designed and generated as described in previous examples. Table 24 shows the ASO sequences designed and generated to target PUFIN8, and these sequences were tested in this example.

TABLE 24
Sequences of ASOs tested in this example to
target PUFIN8.

| | SEQ | | |
|---|---|---|---|
| Figure | ID | Sequence | Modifi- |
| Legends | NO: | (from 5′ to 3′) | cation |
| Scr/Scramble | 2567 | AACACGTCTATACGC | LNA |
| L1 | 2014 | TTAGACCTAGACTGCT | LNA |
| L2 | 2015 | GCCTTAGATCAGTCCT | LNA |
| L3 | 2016 | CCATTAGAGTAGTAGG | LNA |
| L4 | 2017 | GTTACCAGACCTCTAC | LNA |
| L5 | 2018 | GAAACCTTAGTGTGTC | LNA |

Notes:
The L1, L2, L3, L4, and L5 sequences described in this table comprise 3 LNA modified nucleotides at the 5′ end and 2 LNA modified nucleotides at the 3′ end, forming 3-11-2 configuration of LNA-ASO GapmeR. Phosphorothioate linkages were incorporated between each of the nucleotides. Scramble ASO comprises 3 LNA modified nucleotides at the 5′ end and 2 LNA modified nucleotides at the 3′ end, forming 3-10-2 configuration of LNA-ASO GapmeR.

Experiment was performed as described in previous examples.

### Results

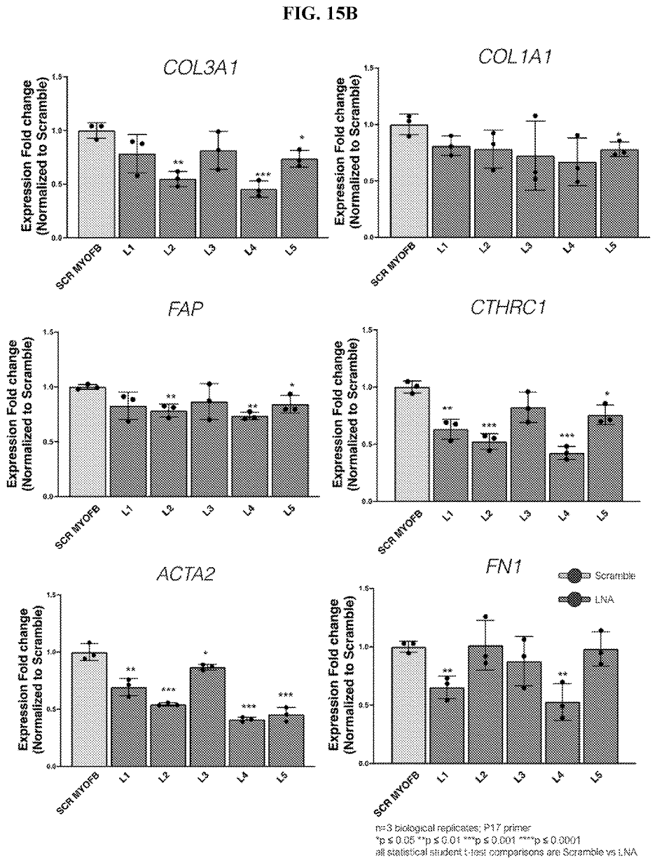

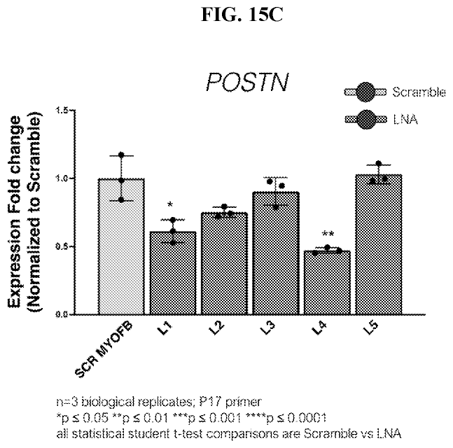

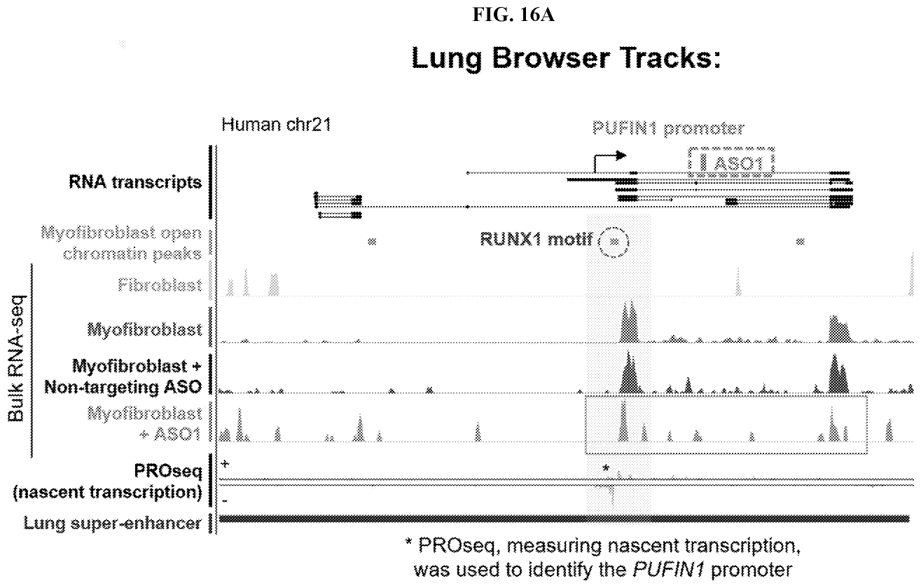

ASOs tested in this example were listed in Table 24. FIG. 15A shows expression of PUFIN8 upon ASOs treatment. FIG. 15B shows expression of COL1A1 in cells treated with ASOs (SEQ ID NO: 2018) was significantly downregulated. Expression of COL3A1 in cells treated with ASOs (SEQ ID NOs: 2015, 2017, 2018) was significantly downregulated. Expression of FAP in cells treated with ASOs (SEQ ID NOs: 2015, 2017, 2018) was downregulated. CTHRC1 expression in cells treated with ASOs (SEQ ID NOs: 2014, 2015, 2017, 2018) was significantly downregulated ACTA2 expression in cells treated with ASOs (SEQ ID NOs: 2014, 2015, 2016, 2017, 2018) was downregulated. FN1 expression in cells treated with ASOs (SEQ ID NOs: 2014, 2017) was downregulated. FIG. 15C shows that POSTN expression in cells treated with ASOs (SEQ ID NOs: 2014, 2017) was downregulated. These results were determined by evaluating the relative expression of the genes via qRT-PCR as described in the previous example. Comparison was performed against Scramble ASO.

### Example 11: Combined TEP Study of ASOs Targeting PUFIN1, PUFIN3, and PUFIN 7

In this example, genes in PUFIN1 TEP, PUFIN3 TEP, and PUFIN7 TEP, as described in the above examples, was combined and further examined.

### Methods:

Further analysis of genes in PUFIN1 TEP, genes in PUFIN3 TEP, and genes in PUFIN7 TEP was performed by 1) overlapping genes among genes in PUFIN1 TEP, genes in PUFIN3 TEP, and genes in PUFIN7 TEP, 2) conducting String DB functional protein interaction analysis, and 3) ENRICHR pathway analysis as described in the previous examples.

### Results:

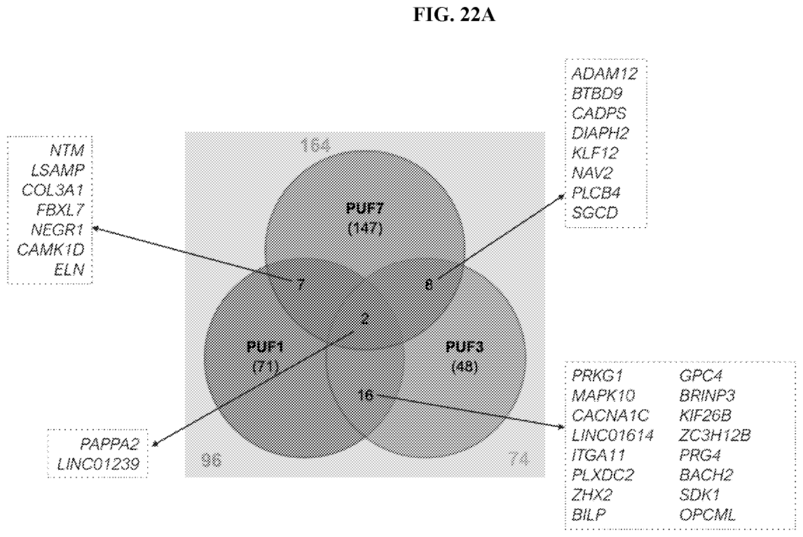

A Venn diagram of the overlapping genes between genes in PUFIN1 TEP, genes in PUFIN3 TEP, and genes in PUFIN7 TEP was plotted (FIG. 22A), and result showed that two genes that were shared among genes listed in PUFIN1 TEP, PUFIN3 TEP, and PUFIN7 TEP were PAPPA2 and LINC01239. Further, seven genes that were shared among genes listed in PUFIN1 TEP and PUFIN7 TEP were NTM, LSAMP, COL3A1, FBXL7, NEGRI, CAMK1D, and ELN. Eight genes that were shared among genes listed in PUFIN7 TEP and PUFIN3 TEP were ADAM12, BTBD9, CADPS, DIAPH2, KLF12, NAV2, PLCB4, and SGCD. 16 genes that were shared among genes listed in PUFIN1 TEP and PUFIN3 TEP were PRKG1, MAPK10, CACNAIC, LINC01614, ITGA11, PLXDC2, ZHX2, BILP, GPC4, BRINP3, KIF26B, ZC3H12B, PRG4, BACH2, SDK1, and OPCML. These genes may be involved in common pathways upon knocking down of PUFINs (FIG. 22A).

Protein-protein interaction network using StringDB in silico analysis of genes in PUFIN1 TEP, genes in PUFIN3 TEP, and genes in PUFIN7 TEP was performed as described in the previous examples and results are shown in Tables 43-44. This network showed the protein-protein interactions in genes from all three PUFIN TEPs and suggested a common network that existed among all three PUFIN TEPs. Table 43 shows genes that were predicted to have protein-protein interaction. Table 44 shows results of co-expression network of genes in PUFIN1 TEP, genes in PUFIN3 TEP, and genes in PUFIN7 TEP.

TABLE 43
Genes in PUFIN1 TEP, PUFIN3 TEP, and PUFIN7 TEP that were
predicted to have protein-protein functional interaction.

| | | | | | |
|---|---|---|---|---|---|
| MMP11 | CCBE1 | GPC6 | PRKG1 | GATA6 | CADM1 |
| SH3PXD | P3H2 | MTS | CCT3 | SMAD2 | PCDH1 |
| 2A | | | | | |
| ADAM12 | COL6A3 | PPFIBP1 | SFRP4 | HMGA2 | LARGE1 |
| TNC | COL3A1 | KCNH1 | LIFR | NEDD9 | RABGA |
| | | | | | P1 |
| ITGA11 | ZBTB20 | OLFM2 | RUNX2 | EHBP1 | EHBP1 |
| COL15A1 | ROR1 | GRIN2A | NLK | CARMI | |
| | | | | L1 | |
| FBN1 | FZD6 | OSBPL3 | KDM3A | JAM3 | |
| ELN | PAPPA2 | CAMK2D | MYOCD | PARD3 | |
| COL14A1 | PTPRD | ITPR1 | STAT3 | MPDZ | |
| COL15A1 | GPC4 | MRVI1 | IGF1 | LIN7A | |

TABLE 44
Genes in co-expression network of PUFIN1 TEP, PUFIN3 TEP, and PUFIN7
TEP.

| | | | | | |
|---|---|---|---|---|---|
| CCBE1 | ZRANB2 | TYRP1 | ADAM12 | MAST2 | PPP1R11 |
| DOK6 | MDC1 | TFEB | TNC | PHF8 | MFAP5 |
| ADAMTS13 | NME7 | PAPPA2 | GPC4 | JADE2 | COL11A1 |
| KCNH1 | JADE3 | PRG4 | SMAD2 | NEDD9 | FBN1 |
| FAM114A2 | EXOC4 | MMP11 | DIF1B | AALADL2 | COL5A2 |
| SEC23IP | MDC1 | SNCAIP | UNC5C | HNRNPU | CHRM2 |
| WDR48 | BBS9 | DYSF | FZD6 | SH3PXD2 | COL15A1 |
| AREL1 | RABGAP1L | P3H2 | PTPRD | ARID2 | MAMDC2 |
| ICA1L | DIAPH2 | ITGA11 | GATA6 | TENM4 | TANGO6 |
| CIA1 | KDM3A | PLN | SBNO2 | CRIN2A | AGK |
| HPCAL1 | PLXNA4 | COL6A2 | STAT3 | CACNA1C | IMMP2L |
| HMCN1 | DUSP16 | CAMK2D | PRICKLE2 | PAPPA | SND1 |
| LARGE1 | RSAD2 | ADORA1 | ZBTB20 | HNRNPL | OLFM2 |
| MRVI1 | JAM3 | CILP | NFYA | NOVA1 | THSD7A |
| ITGA11 | ROR1 | RNDC1 | ABCC1 | AAK1 | DNAJC8 |
| PLN | SRGAP1 | COL14A1 | ZDHHC16 | HMGA2 | DENND1A |
| COL6A3 | SMYD3 | COL3A1 | PPFIBP1 | ETV6 | SMARCAD1 |
| SPON1 | MX2 | LIN7A | CAMK1D | LPP | TIA1 |
| CAMK2D | SLC7A6 | GLCE | SRGAP1 | CCT3 | COL5A1 |
| ASTN2 | SLC7A11 | CAPN1 | RALGAPA2 | PHF8 | ITPR1 |
| MXRA5 | BACH2 | SLC8A1 | ROBO2 | VIRMA | TNC |
| DNAJC8 | LIFR | FRMPD4 | CADM1 | BTBD9 | PRKG1 |
| DENND1A | MTSS1 | CALD1 | ROBO1 | WDR70 | |
| SMARCAD1 | CADPS | SFRP4 | GAS7 | ZHX2 | |
| TIA1 | LSAMP | OPCML | PARD3 | EXT1MYOCD | |
| RNF150 | GPC6 | SMAD2 | SH3YL1 | SGCD | |
| NUP133 | RUNX2 | IGF1 | MAPKAPK2 | PCDH1 | |

Transcription factor enrichment analysis was performed using combined genes in PUFIN1 TEP, genes in PUFIN3 TEP, and genes in PUFIN7 TEP. Top factors were SMAD4, SMARCD1, WT1, STAT3, ESR1, NFκB1, TCF4, CTNNB1, YAP1, and AR. ENRICHR analysis of the top Reactome Gene Ontology Pathways associated with combined genes in PUFIN1 TEP, genes in PUFIN3 TEP, and genes in PUFIN7 TEP was performed, and results show that top pathways were extracellular matrix organization; collagen chain trimerization; collagen biosynthesis and modifying enzymes; defective EXT1 causes exostoses 1, TRPS2, and CHDS; HS-GAG biosynthesis; cGMP effects; diseases of glycosylation; Ca2+ pathway; collagen formation; signaling by PDGFR in disease. Gene Ontology (GO) Pathway Analysis based on GO Molecular Processes, GO Cellular Components, and GO Biological Processes, revealing basal membrane and cell organization pathways were key pathway associated with combined PUFIN1 TEP, PUFIN3 TEP, and PUFIN7 TEP. Top pathways in GO molecular processes comprised phosphatidylinositol bisphosphate binding; phosphatidylinositol-4,5-bisphosphate binding; transmembrane receptor protein phosphatase activity; transmembrane receptor protein tyrosine phosphatase activity; coreceptor activity involved in Wnt signaling pathway, planar cell polarity pathway; coreceptor activity involved in Wnt signaling pathway; calmodulin-dependent protein kinase activity; phosphatidylinositol binding; potassium: chloride symporter activity; metal ion transmembrane transporter activity. Top pathways in GO cellular components comprised basement membrane; collagen-containing extracellular matrix; platelet dense tubular network membrane; platelet dense tubular network; endoplasmic reticulum lumen; supramolecular fiber; protein kinase complex; neuron projection; sarcolemma. Top pathways in GO biological processes comprised positive regulation of cell projection organization; establishment of protein localization to extracellular region; relaxation of cardiac muscle; relaxation of muscle; cell-cell adhesion via plasma-membrane adhesion molecules; inositol lipid-mediated signaling; calcium ion transmembrane import into cytosol; regulation of cardiac muscle contraction by regulation of the release of sequestered calcium ion; muscle organ development; homophilic cell adhesion via plasma membrane adhesion molecules. ENRICHR analysis of top Wikipathways associated with combined genes in PUFIN1 TEP, genes in PUFIN3 TEP, and genes in PUFIN7 TEP, and result is shown in Table 45. The top Wikipathways were significantly dominated by Wnt Signaling pathways. Key genes identified were CAMK2D, GPC4, FZD6, NLK, PLCB1, and PLCB4.

TABLE 45
Wikipathways associated with genes from the PUFIN1 TEP,
PUFIN3 TEP, and PUFIN7 TEP.

| | | | Ad- | | Com- |
|---|---|---|---|---|---|
| In- | | | justed | Odds | bined |
| dex | Name | P-value | p-value | Ratio | score |
| 1 | Wnt Signaling | 0.00005436 | 0.01729 | 5.79 | 56.88 |
| | WP428 | | | | |
| 2 | Vitamin D | 0.0001862 | 0.0296 | 10.79 | 92.68 |
| | Sensitive | | | | |
| | Calcium | | | | |
| | Signaling in | | | | |
| | Depression | | | | |
| | WP4698 | | | | |
| 3 | miRNA | 0.0002895 | 0.03069 | 14.83 | 120.8 |
| | Targets In | | | | |
| | ECM and | | | | |
| | Membrane | | | | |
| | Receptors | | | | |
| | WP2911 | | | | |
| 4 | GPR143 In | 0.0009874 | 0.0785 | 10.26 | 71.01 |
| | Melanocytes | | | | |
| | and Retinal | | | | |
| | Pigment | | | | |
| | Epithelium | | | | |
| | Cells | | | | |
| | WP4941 | | | | |
| 5 | TGF Beta | 0.001926 | 0.1021 | 14.25 | 89.11 |
| | Signaling In | | | | |
| | Thyroid | | | | |
| | Cells for | | | | |
| | Epithelial | | | | |
| | Mesenchymal | | | | |
| | Transition | | | | |
| | WP3859 | | | | |
| 6 | mR509 3P | 0.001926 | 0.1021 | 14.25 | 73.82 |
| | Alteration of | | | | |
| | YAP1 ECM | | | | |
| | Axis WP3967 | | | | |
| 7 | Extracellular | 0.002684 | 0.1219 | 12.47 | 57.82 |
| | Vesicles in the | | | | |
| | Crosstalk of | | | | |
| | Cardiac | | | | |
| | Cells WP4300 | | | | |
| 8 | PKC Gamma | 0.004127 | 0.164 | 10.5 | 57.64 |
| | Calcium | | | | |
| | Signaling | | | | |
| | Pathway in | | | | |
| | Ataxia | | | | |
| | WP4760 | | | | |
| 9 | Neuroinflam- | 0.005161 | 0.1687 | 3.53 | 18.58 |
| | mation and | | | | |
| | Glutamatergic | | | | |
| | Signaling | | | | |
| | WP5083 | | | | |
| 10 | Physiological | 0.005306 | 0.1687 | 9.5 | 49.76 |
| | And Patholog- | | | | |
| | ical hypertrophy | | | | |
| | of the | | | | |
| | Heart WP1528 | | | | |

LINC01614, which is a lncRNA, was identified in both PUFIN1 TEP and in PUFIN3 TEP. LINC01614 and PUFIN7 share overlapped genomic locations. Thus, expression of PUFIN7 was analyzed in cells treated with PUFIN1 ASO (SEQ ID NO: 36) and in cells treated with PUFIN3 ASO (SEQ ID NO: 10). As shown in FIGS. 23A-23B, expression of PUFIN7 responded in a dose dependent manner upon PUFIN1 ASO1 (SEQ ID NO: 36) treatment (FIG. 23A) and upon PUFIN3 L1 ASO (SEQ ID NO: 10) treatment (FIG. 23B), suggesting that lncRNAs, e.g., PUFIN7, may be involved in common pathways or networks with other lncRNAs, e.g., PUFIN1 or PUFIN3. This results show that lncRNAs can modulate/affect not only expression of protein-coding genes but also expression of other lncRNAs.

LINC01239, which is a lncRNA, was found to be downregulated in a dose dependent manner upon PUFIN1 ASO1 (SEQ ID NO: 36) treatment, PUFIN3 L1 ASO (SEQ ID NO: 10) treatment, and PUFIN7 L6 ASO (SEQ ID NO: 32) treatment, as shown in FIG. 23C. Information regarding LINC01239 is shown in Table 34.

TABLE 34
Information of LINC01239 or PUFIN13

| | | | | | Start | End |
|---|---|---|---|---|---|---|
| Name | Gene ID | Gene Name | Strand | Chromosome | location | location |
| PUFIN13 | ENSG00000234840.2 | LINC01239 | + | Chr9 | 22646200 | 22824213 |

Upon examination, LINC01239 (i.e., PUFIN13) was reported to be in the same regulatory network as identified in the Example 1, suggesting that this lncRNA, LINC01239, can be used as a biomarker for other PUFINs or for IPF.

### Example 12. Identification of IPF Risk Variants Associated with Genomic Locations of PUFIN Transcripts

Genome-wide association studies (GWAS) of IPF susceptibility identified common variants (minor allele frequency [MAF]>5%) near genes that are involved in host defense, telomere maintenance, cell-cell adhesion and signaling in disease susceptibility. In this example, GWAS IPF risk variants associated with genomic locations of PUFIN transcripts were identified.

Methods: A genome-wide association study (GWAS) using the genomic dataset from Allen, R. et al. 2021 medRxiv (“Genome wide association study across five cohorts identifies five novel loci associated with idiopathic pulmonary fibrosis”) was analyzed to identify the single nucleotide polymorphisms (SNP) variants, and their associations with survival rates of idiopathic pulmonary fibrosis patient. The dataset included a list of SNPs and their association with IPF patient survival (which had been mapped to human genome hg19 coordinates). The data in hg19 coordinates was converted to human genome hg38. SNPs variants overlapping with PUFIN loci were extracted.

Results: IPF risk variants associated with PUFIN loci were analyzed and identified as shown in Tables 46-57. Some of these SNPs are located in target regions of interest for design and generation of modulators, such as ASOs. The presence of SNPs associated with IPF patient survival rate located within PUFIN loci serves as additional genetic evidence that the genomic loci transcribing PUFIN and PUFIN expression may impact human IPF pathology.

### Example 13. Identification of IPF Transplant Free Survival Variants Associated with Genomic Locations of PUFIN Transcripts

A few IPF susceptibility-associated variants have been shown to predict IPF outcomes, suggesting that genomic determinants of IPF susceptibility and survival may have limited overlap. In 150his example, GWAS IPF transplant free survival variants associated with genomic locations of PUFIN transcripts were identified.

Methods: In this example, a GWAS using the genomic dataset from Oldham, J. et al., 2023 Am J Respir Crit Care Med. (“PCSK6 and Survival in Idiopathic Pulmonary Fibrosis”) was analyzed to identify SNP variants. Similar methods as described in Example 12 were performed.

Results: IPF transplant free survival variants associated with PUFIN loci were analyzed and identified as shown in Tables 58-69. Some of these SNPs are located in target regions of interest for design and generation of modulators, such as ASOs. The presence of SNPs associated with IPF patient survival rate located within PUFIN loci serves as additional genetic evidence that the genomic loci transcribing PUFIN and PUFIN expression may impact human IPF pathology.

### Example 14. ASOs Targeting SNPs Associated with Genomic Locations of PUFIN Transcripts

Based on identification of IPF risk variants and transplant free survival variants as describe in Examples 12-13, ASOs were designed to target these sites.

Tables 70-77 show lists of ASOs targeting SNPs associated with each PUFIN lncRNAs.

To determine efficacy and target specificity, ASOs are tested in vitro and/or in vivo as described in the previous examples. Additional tests are performed to evaluate other parameters, e.g., efficacy, specificity, and/or toxicity.

While preferred embodiments of the present invention have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the instances herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

### List of Exemplary Embodiments

Embodiment 1. A modulator of a long noncoding RNA (lncRNA), wherein the lncRNA is transcribed from a region located within a genomic locus selected from any one from shown in Table 17 or Table 34: ENSG00000237232.9 (chr21: 42009177-42055325), ENSG00000259345.7 (chr15: 38864806-39427195), XLOC_029593 (chr15: 38871616-38880364), ENSG00000261039.3 (chr6: 169171312-169188635), ENSG00000224218.1 (chr4: 76758554-76801964), ENSG00000249406.3 (chr17: 50199876-50215922), ENSG00000230838.2 (chr2: 215717811-215721161), ENSG00000258425.1 (chr14: 74552181-74560688), XLOC_051798 (chr2: 188966458-188984794), ENSG00000286275.1 (chr11: 1218530-1220242), ENSG00000223786.1 (chr6: 74069451-74690727), ENSG00000289423.1 (chr16: 86845338-87058332), and ENSG00000234840.2 (chr9: 22646200-22824213).

Embodiment 2. The modulator of embodiment 2, wherein RNA expression level of the lncRNA is higher in pulmonary myofibroblasts compared to RNA expression level of the lncRNA in pulmonary fibroblasts.

Embodiment 3. The modulator of any one of embodiment 1 or embodiment 2, wherein the RNA expression level of the lncRNA is at least about 2 folds, at least about 10 folds, or at least about 50 folds higher in the pulmonary myofibroblasts than in the pulmonary fibroblasts.

Embodiment 4. The modulator of any one of embodiments 1-3, wherein RNA expression level of the lncRNA is higher in pulmonary myofibroblasts obtained from a subject with IPF compared to RNA expression level of the lncRNA in pulmonary myofibroblasts obtained from a healthy subject.

Embodiment 5. The modulator of any one of embodiments 1-4, wherein the RNA expression level of the lncRNA is at least about 2 folds, at least about 10 folds, or at least about 50 folds higher in the pulmonary myofibroblasts obtained from the subject with IPF than in the pulmonary myofibroblasts obtained from the healthy subject.

Embodiment 6. The modulator of any one of embodiments 1-5, wherein the lncRNA is a pulmonary-specific lncRNA.

Embodiment 7. The modulator of any one of embodiments 1-6, wherein the lncRNA is associated with pathology of IPF.

Embodiment 8. The modulator of any one of embodiments 1-7, wherein genomic locations encoding the lncRNA comprises one or more genetic variants.

Embodiment 9. The modulator of embodiment 8, wherein the one or more genetic variants comprise one or more single nucleotide polymorphisms (SNPs), indel variations, copy number variations (CNV), or combination thereof.

Embodiment 10. The modulator of embodiment 9, wherein the one or more SNPs are associated with susceptibility to pulmonary fibrosis (e.g., IPF), immune function (lymphocyte count), lung function (e.g., forced vital capacity), systolic blood pressure, response to taxane treatment (e.g., docetaxel), or a combination thereof.

Embodiment 11. The modulator of any one of embodiment 9 or embodiment 10, wherein the one or more SNPs comprises rs150797, rs220262, rs220249, rs2292305, rs3763267, rs2586502, rs61980882, rs37505950, or combination thereof.

Embodiment 12. The modulator of any one of embodiments 1-11, wherein the modulator is a nucleic acid editing or modifying tool.

Embodiment 13. The modulator of any one of embodiments 1-12, wherein the modulator is a synthetic or artificial oligonucleotide.

Embodiment 14. The modulator of embodiment 13, wherein the synthetic or artificial oligonucleotide comprises a nucleic acid sequence complementary to at least 10, 11, 12, 13, 14, or 15 nucleotides of the lncRNA.

Embodiment 15. The modulator of any one of embodiment 13 or embodiment 14, wherein the synthetic or artificial oligonucleotide comprises at least 13 nucleotides.

Embodiment 16. The modulator of any one of embodiments 13-15, wherein the synthetic or artificial oligonucleotide comprises at most 35 nucleotides.

Embodiment 17. The modulator of any one of embodiments 13-16, wherein the synthetic or artificial oligonucleotide is about 13-35 nucleotides.

Embodiment 18. The modulator of any one of embodiments 13-16, wherein the synthetic or artificial oligonucleotide is about 16-20 nucleotides.

Embodiment 19. The modulator of any one of embodiments 13-18, wherein the synthetic or artificial oligonucleotide comprises a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), or an enzymatically-prepared siRNA (esiRNA) or a precursor thereof.

Embodiment 20. The modulator of any one of embodiments 13-19, wherein the synthetic oligonucleotide comprises one or more sugar modifications, one or more phosphate backbone modifications, one or more base modifications, or any combination thereof.

Embodiment 21. The modulator of embodiment 20, wherein the one or more sugar modifications are locked nucleic acid (LNA), tricyclo-DNA, 2′-fluoro, 2′-O-methyl, 2′-methoxyethyl(2′-MOE), 2′ cyclic ethyl (CET), UNA, and conformationally restricted nucleoside (CRN), or any combination thereof.

Embodiment 22. The modulator of any one of embodiment 20 or embodiment 21, wherein the one or more phosphate backbone modifications comprise phosphorothioate internucleotide linkage, methylphosphonate internucleotide linkage, guanidinopropyl phosphoramidate internucleotide linkage, or any combination thereof.

Embodiment 23. The modulator of any one of embodiments 20-22, wherein the one or more base modifications comprise a purine modification selected from a group consisting of 2,6-diaminopurin, 3-deaza-adenine, 7-deaza-guanine, 8-zaido-adenine, or any combination thereof.

Embodiment 24. The modulator of any one of embodiments 20-23, wherein the one or more base modifications comprise a pyrimidine modification selected from a group consisting of 2-thio-thymidine, 5-carboxamide-uracil, 5-methyl-cytosine, 5-ethynyl-uracil, or any combination thereof.

Embodiment 25. The modulator of any one of embodiments 13-24, wherein the synthetic oligonucleotide is an ASO, wherein the ASO comprises i) a GapmeR comprising a central region of consecutive DNA nucleotides flanked by a 5′-wing region and 3′-wing region, wherein at least one of 5′-wing region and 3′-wing region comprises at least one nucleic acid analogue; and/or ii) a MixmeR of DNA and at least one nucleic acid analogue, or a combination thereof.

Embodiment 26. The modulator of embodiment 25, wherein the at least one nucleic acid analogue comprises an LNA.

Embodiment 27. The modulator of embodiment 26, wherein the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA.

Embodiment 28. The modulator of any one of embodiments 25-27, wherein the 5′-wing region comprises at least two LNAs.

Embodiment 29. The modulator of embodiment 28, wherein the 5′-wing region comprises three consecutive LNAs.

Embodiment 30. The modulator of any one of embodiments 25-29, wherein the 3′-wing region comprises at least one LNA.

Embodiment 31. The modulator of embodiment 30, wherein the 3′-wing region comprises two consecutive LNAs.

Embodiment 32. The modulator of any one of embodiments 26-31, wherein the central region of consecutive DNA nucleotides comprises at least 8, 9, or 10 DNA nucleotides.

Embodiment 33. The modulator of embodiment 32, wherein the central region of consecutive DNA nucleotides comprises 11 DNA nucleotides.

Embodiment 34. The modulator of any one of embodiments 26-33, wherein at least one internucleotide linkage of the ASO is a phosphorothioate internucleotide linkage.

Embodiment 35. The modulator of embodiment 34, each internucleotide linkage of the ASO is a phosphorothioate internucleotide linkage.

Embodiment 36. The modulator of embodiment 25, wherein the at least one nucleic acid analogue comprises a 2′-methoxyethyl (MOE)-modified nucleotide.

Embodiment 37. The modulator of embodiment 36, wherein the 5′-wing region comprises at least 2, 3, or 4 MOE-modified nucleotides.

Embodiment 38. The modulator of embodiment 37, wherein the 5′-wing region comprises 5 consecutive MOE-modified nucleic acids.

Embodiment 39. The modulator of any one of embodiments 36-38, wherein the 3′-wing region comprises at least 2, 3, or 4 MOE-modified nucleotides.

Embodiment 40. The modulator of embodiment 39, wherein the 3′-wing region comprises 5 consecutive MOE-modified nucleotides.

Embodiment 41. The modulator of any one of embodiments 36-40, wherein the central region of consecutive DNA nucleotides comprises at least 8 or 9 DNA nucleotides.

Embodiment 42. The modulator of embodiment 41, wherein the central region of consecutive DNA nucleotides comprises 10 DNA nucleotides.

Embodiment 43. The modulator of any one of embodiments 36-42, wherein at least one internucleotide linkage of the ASO is a phosphorothioate internucleotide linkage.

Embodiment 44. The modulator of embodiment 43, wherein each internucleotide linkage of the ASO is a phosphorothioate internucleotide linkage.

Embodiment 45. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 1-2566 and 2580-3987.

Embodiment 46. The modulator of any one of embodiments 1-44, wherein the modulator comprises a nucleic acid sequence comprising at least 80%, at least 85%, at least 90%, or at least 95% identical to a sequence selected from any one of SEQ ID NOs: 1-2566 and 2580-3987.

Embodiment 47. The modulator of any one of embodiments 1-44, wherein the modulator comprises a nucleic acid sequence consisting of a sequence selected from any one of SEQ ID NOs: 1-2566 and 2580-3987.

Embodiment 48. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 1-28, 31-37, 378, 385, 387, 2010-2018, and 2564-2566.

Embodiment 49. The modulator of any one of embodiments 1-44, wherein the modulator comprises a nucleic acid sequence comprising at least 80%, at least 85%, at least 90%, or at least 95% identical to a sequence selected from any one of SEQ ID NOs: 1-28, 31-37, 378, 385, 387, 2010-2018, and 2564-2566.

Embodiment 50. The modulator of any one of embodiments 1-44, wherein the modulator comprises a nucleic acid sequence consisting of a sequence selected from any one of SEQ ID NOs: 1-28, 31-37, 378, 385, 387, 2010-2018, and 2564-2566.

Embodiment 51. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 36-40 and 1406-1968.

Embodiment 52. The modulator of any one of embodiments 1-44, wherein the modulator comprises a nucleic acid sequence comprising at least 80%, at least 85%, at least 90%, or at least 95% identical to a sequence selected from any one of SEQ ID NOs: 36-40 and 1406-1968.

Embodiment 53. The modulator of any one of embodiments 1-44, wherein the modulator comprises a nucleic acid sequence consisting of a sequence selected from any one of SEQ ID NOs: 36-40 and 1406-1968.

Embodiment 54. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 1-9, 1969-2009, and 2564-2566.

Embodiment 55. The modulator of any one of embodiments 1-44, wherein the modulator comprises a nucleic acid sequence comprising at least 80%, at least 85%, at least 90%, or at least 95% identical to a sequence selected from any one of SEQ ID NOs: 1-9, 1969-2009, and 2564-2566.

Embodiment 56. The modulator of any one of embodiments 1-44, wherein the modulator comprises a nucleic acid sequence consisting of a sequence selected from any one of SEQ ID NOs: 1-9, 1969-2009, and 2564-2566.

Embodiment 57. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 10-16 and 802-1405.

Embodiment 58. The modulator of any one of embodiments 1-44, wherein the modulator comprises a nucleic acid sequence comprising at least 80%, at least 85%, at least 90%, or at least 95% identical to a sequence selected from any one of SEQ ID NOs: 10-16 and 802-1405.

Embodiment 59. The modulator of any one of embodiments 1-44, wherein the modulator comprises a nucleic acid sequence consisting of a sequence selected from any one of SEQ ID NOs: 10-16 and 802-1405.

Embodiment 60. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 2010-2013.

Embodiment 61. The modulator of any one of embodiments 1-44, wherein the modulator comprises a nucleic acid sequence comprising at least 80%, at least 85%, at least 90%, or at least 95% identical to a sequence selected from any one of SEQ ID NOs: 2010-2013.

Embodiment 62. The modulator of any one of embodiments 1-44, wherein the modulator comprises a nucleic acid sequence consisting of a sequence selected from any one of SEQ ID NOs: 2010-2013.

Embodiment 63. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 17-26 and 41-559.

Embodiment 64. The modulator of any one of embodiments 1-44, wherein the modulator comprises a nucleic acid sequence comprising at least 80%, at least 85%, at least 90%, or at least 95% identical to a sequence selected from any one of SEQ ID NOs: 17-26 and 41-559.

Embodiment 65. The modulator of any one of embodiments 1-44, wherein the modulator comprises a nucleic acid sequence consisting of a sequence selected from any one of SEQ ID NOs: 17-26 and 41-559.

Embodiment 66. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 27-35 and 560-801.

Embodiment 67. The modulator of any one of embodiments 1-44, wherein the modulator comprises a nucleic acid sequence comprising at least 80%, at least 85%, at least 90%, or at least 95% identical to a sequence selected from any one of SEQ ID NOs: 27-35 and 560-801.

Embodiment 68. The modulator of any one of embodiments 1-44, wherein the modulator comprises a nucleic acid sequence consisting of a sequence selected from any one of SEQ ID NOs: 27-35 and 560-801.

Embodiment 69. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 2014-2018.

Embodiment 70. The modulator of any one of embodiments 1-44, wherein the modulator comprises a nucleic acid sequence comprising at least 80%, at least 85%, at least 90%, or at least 95% identical to a sequence selected from any one of SEQ ID NOs: 2014-2018.

Embodiment 71. The modulator of any one of embodiments 1-44, wherein the modulator comprises a nucleic acid sequence consisting of a sequence selected from any one of SEQ ID NOs: 2014-2018.

Embodiment 72. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 2019-2073.

Embodiment 73. The modulator of any one of embodiments 1-44, wherein the modulator comprises a nucleic acid sequence comprising at least 80%, at least 85%, at least 90%, or at least 95% identical to a sequence selected from any one of SEQ ID NOs: 2019-2073.

Embodiment 74. The modulator of any one of embodiments 1-44, wherein the modulator comprises a nucleic acid sequence consisting of a sequence selected from any one of SEQ ID NOs: 2019-2073.

Embodiment 75. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 2074-2076.

Embodiment 76. The modulator of any one of embodiments 1-44, wherein the modulator comprises a nucleic acid sequence comprising at least 80%, at least 85%, at least 90%, or at least 95% identical to a sequence selected from any one of SEQ ID NOs: 2074-2076.

Embodiment 77. The modulator of any one of embodiments 1-44, wherein the modulator comprises a nucleic acid sequence consisting of a sequence selected from any one of SEQ ID NOs: 2074-2076.

Embodiment 78. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 2077-2310.

Embodiment 79. The modulator of any one of embodiments 1-44, wherein the modulator comprises a nucleic acid sequence comprising at least 80%, at least 85%, at least 90%, or at least 95% identical to a sequence selected from any one of SEQ ID NOs: 2077-2310.

Embodiment 80. The modulator of any one of embodiments 1-44, wherein the modulator comprises a nucleic acid sequence consisting of a sequence selected from any one of SEQ ID NOs: 2077-2310.

Embodiment 81. The modulator of any one of embodiments 1-44, wherein the modulator comprises a nucleic acid molecule comprising at least 10, at least 11, at least 12, or at least 13 consecutive nucleotides with no more than 1, no more than 2, or no more than 3 mismatches from any one of SEQ ID NOs: 2311-2563.

Embodiment 82. The modulator of any one of embodiments 1-44, wherein the modulator comprises a nucleic acid sequence comprising at least 80%, at least 85%, at least 90%, or at least 95% identical to a sequence selected from any one of SEQ ID NOs: 2311-2563.

Embodiment 83. The modulator of any one of embodiments 1-44, wherein the modulator comprises a nucleic acid sequence consisting of a sequence selected from any one of SEQ ID NOs: 2311-2563.

Embodiment 84. The modulator of embodiment 12, wherein the modulator comprises an endonuclease complex guided by a nucleic acid, wherein the nucleic acid targets the lncRNA or a genomic locus thereof.

Embodiment 85. The modulator of embodiment 84, wherein the modulator is a CRISPR-directed DNA editing complex or a CRISPR-directed RNA editing complex.

Embodiment 86. The modulator of any one of embodiments 1-85, wherein the modulator targets a region of the lncRNA, wherein the region of the lncRNA comprises a conserved exon shared among a plurality of isoforms of the lncRNA.

Embodiment 87. A pharmaceutical composition comprising the modulator of any one of embodiments 1-86 and a pharmaceutically acceptable salt, excipient, or derivative thereof.

Embodiment 88. The pharmaceutical composition of embodiment 87, wherein the modulator is encapsulated in liposome or coupled with a nanoparticle.

Embodiment 89. A pharmaceutical composition comprising an expression vector, wherein the expression vector comprises a transgene, wherein the transgene encodes the modulator of any one of embodiments 1-86.

Embodiment 90. A kit comprising the modulator of any one of embodiments 1-86 or the pharmaceutical composition of any one of embodiments 87-89.

Embodiment 91. A method of modulating one or more pulmonary-specific lncRNAs, comprising administering an effective amount of the modulator of any one of embodiments 1-86 or the pharmaceutical composition of any one of embodiments 87-89 to a subject in need thereof, wherein the one or more pulmonary-specific lncRNAs are transcribed from a region located within a genomic locus selected from any one from shown in Table 17 or Table 34: ENSG00000237232.9 (chr21: 42009177-42055325), ENSG00000259345.7 (chr15: 38864806-39427195), XLOC_029593 (chr15: 38871616-38880364), ENSG00000261039.3 (chr6: 169171312-169188635), ENSG00000224218.1 (chr4: 76758554-76801964), ENSG00000249406.3 (chr17: 50199876-50215922), ENSG00000230838.2 (chr2: 215717811-215721161), ENSG00000258425.1 (chr14: 74552181-74560688), XLOC_051798 (chr2: 188966458-188984794), ENSG00000286275.1 (chr11: 1218530-1220242), ENSG00000223786.1 (chr6: 74069451-74690727), ENSG00000289423.1 (chr16: 86845338-87058332), and ENSG00000234840.2 (chr9: 22646200-22824213).

Embodiment 92. The method of embodiment 91, wherein the modulator reduces RNA expression level of the pulmonary-specific lncRNAs.

Embodiment 93. The method of any one of embodiment 91 or embodiment 92, wherein the modulator further modulates RNA expression level, protein expression level, or both, of one or more genes from Table 18.

Embodiment 94. The method of embodiment any one of embodiments 91-93, wherein the modulator reduces RNA expression level, protein expression level, or both, of the one or more genes from Table 18.

Embodiment 95. The method of any one of embodiments 91-94, wherein the modulator modulates RNA expression level, protein expression level, or both, of subsets of genes associated with autophagy, ubiquitin, cilium and flagellum assembly and function, extracellular matrix (e.g., collagen) synthesis, metabolism, deposition, and organization, cell organization, genome regulation, or ncRNA and mRNA regulation.

Embodiment 96. The method of any one of embodiments 91-95, wherein the modulator reduces RNA expression level, protein expression level, or both, of one or more fibrotic genes.

Embodiment 97. The method of embodiment 96, wherein the one or more fibrotic genes comprise COL3A1, COL1A1, ACTA2, POSTN, FN1, FAP, CTHRC1, or combination thereof.

Embodiment 98. The method of any one of embodiments 91-97, wherein the modulator further modulates one or more genes detected in co-expression module.

Embodiment 99. The method of embodiment 98, wherein the one or more genes are MX1, MX2, thrombospondin 1 (THBS1), thrombospondin 2 (THBS2), or combination thereof.

Embodiment 100. The method of embodiment 98, wherein the one or more genes are B9D1, IQCG, LRRC23, TMEM231, SPACA9, CCDC65, ARHGAP39, C9orf116, CFAP300, RAB36, or a combination thereof.

Embodiment 101. The method of embodiment 98, wherein the one or more genes are CCDC60, FAM81B, TCTE1, MDH1B, RSPH9, PACRG, C9orf116, ODAD4, TMEM231, or a combination thereof.

Embodiment 102. The method of embodiment 98, wherein the one or more genes are NTN1, TMEM45A, GOLM1, SERPINB5, FAT2, SPTBN2, or a combination thereof.

Embodiment 103. The method of embodiment 98, wherein the one or more genes are CERCAM, COL3A1, COL1A1, COL5A2, COL1A2, CIS, ROR2, EPHB2, COL15A1, CTHRC1, or a combination thereof.

Embodiment 104. The method of any one of embodiments 91-103, wherein the modulator restores a cytokine level that is similar to a reference cytokine level obtained from a population of healthy subjects.

Embodiment 105. The method of embodiment 104, wherein the cytokine comprises IL-9, IL-1a, MIP-1a (CCL3), G-CSF, IL-2, IL-7, or a combination thereof.

Embodiment 106. The method of any one of embodiments 91-105, wherein the modulator inhibits epithelial to mesenchymal transition (EMT) of pulmonary cells.

Embodiment 107. The method of embodiment 106, wherein the pulmonary cells comprise pulmonary fibroblasts, pulmonary myofibroblasts, or epithelial cells.

Embodiment 108. The method of any one of embodiments 91-107, wherein the administering is performed intratracheally, orally, nasally, intravenously, intraperitoneally, or intramuscularly.

Embodiment 109. The method of any one of embodiments 91-108, wherein the administering is a targeted delivery to a lung tissue of the subject.

Embodiment 110. The method of any one of embodiments 91-109, wherein the administering is in a form of aerosol.

Embodiment 111. A method of treating idiopathic pulmonary fibrosis (IPF) in a subject, the method comprising: (a) obtaining a sample from the subject; (b) measuring RNA expression level, protein expression level, or both, of a set of genes setting forth in Table 18; (c) measuring RNA expression level of one or more pulmonary-specific lncRNAs, wherein the one or more pulmonary-specific lncRNAs are transcribed from a region located within a genomic locus selected from any one of ENSG00000237232.9 (chr21: 42009177-42055325), ENSG00000259345.7 (chr15: 38864806-39427195), XLOC_029593 (chr15: 38871616-38880364), ENSG00000261039.3 (chr6: 169171312-169188635), ENSG00000224218.1 (chr4: 76758554-76801964), ENSG00000249406.3 (chr17: 50199876-50215922), ENSG00000230838.2 (chr2: 215717811-215721161), ENSG00000258425.1 (chr14: 74552181-74560688), XLOC_051798 (chr2: 188966458-188984794), ENSG00000286275.1 (chr11: 1218530-1220242), ENSG00000223786.1 (chr6: 74069451-74690727), ENSG00000289423.1 (chr16: 86845338-87058332), and ENSG00000234840.2 (chr9: 22646200-22824213); (d) diagnosing the subject with IPF; and (e) treating the subject with the modulator of any one of embodiments 1-86 or the pharmaceutical composition of any one of embodiments 87-89.

Embodiment 112. A method of predicting severity and progression of idiopathic pulmonary fibrosis (IPF) in a subject, the method comprising: (a) obtaining a sample from the subject; (b) measuring an RNA expression level of a set of genes comprising COL3A1, COL1A1, ACTA2, POSTN, FN1, FAP, CTHRC1, THBS1, THBS2, or a combination thereof; (c) measuring an RNA expression level of one or more pulmonary-specific lncRNAs, wherein the one or more pulmonary-specific lncRNAs are transcribed from a region located within a genomic locus selected from any one of ENSG00000237232.9 (chr21: 42009177-42055325), ENSG00000259345.7 (chr15: 38864806-39427195), XLOC_029593 (chr15: 38871616-38880364), ENSG00000261039.3 (chr6: 169171312-169188635), ENSG00000224218.1 (chr4: 76758554-76801964), ENSG00000249406.3 (chr17: 50199876-50215922), ENSG00000230838.2 (chr2: 215717811-215721161), ENSG00000258425.1 (chr14: 74552181-74560688), XLOC_051798 (chr2: 188966458-188984794), ENSG00000286275.1 (chr11: 1218530-1220242), ENSG00000223786.1 (chr6: 74069451-74690727), ENSG00000289423.1 (chr16: 86845338-87058332), and ENSG00000234840.2 (chr9: 22646200-22824213); and (d) computing (i) an increased RNA expression level of the set of genes from (b) in comparison to a first reference RNA expression level and (ii). an increased RNA expression level of the one or more pulmonary-specific lncRNAs from (c) in comparison to a second reference RNA expression level, thereby predicting severity and progression of IPF in the subject.

Embodiment 113. The method of embodiment 112, wherein the one or more pulmonary-specific lncRNAs are transcribed from a region located within a genomic locus selected from any one of ENSG00000237232.9 (chr21: 42009177-42055325), ENSG00000259345.7 (chr15: 38864806-39427195), XLOC_029593 (chr15: 38871616-38880364), ENSG00000261039.3 (chr6: 169171312-169188635), ENSG00000249406.3 (chr17: 50199876-50215922), and ENSG00000230838.2 (chr2: 215717811-215721161).

Embodiment 114. The method of any one of embodiment 112 or embodiment 113, wherein the one or more pulmonary-specific lncRNAs are transcribed from a region located within a genomic locus selected from any one of ENSG00000259345.7 (chr15: 38864806-39427195) and XLOC_029593 (chr15: 38871616-38880364).

Embodiment 115. The method of any one of embodiments 112-114, wherein the increased RNA expression level of the one or more pulmonary-specific lncRNAs from (c) is at least about 0.5-fold, or at least about 10 folds higher in comparison to the second reference RNA expression level.

Embodiment 116. The method of any one of embodiments 112-115, wherein the first reference RNA expression level and the second reference RNA expression level are obtained from a control.

Embodiment 117. The method of any one of embodiments 112-116, further comprising administering to the subject the modulator of any one of embodiments 1-85 or the pharmaceutical composition of any one of embodiments 87-89.

TABLE 1
Sequences of ASO candidates targeting PUFIN2.

| SEQ ID | | SEQ ID | | SEQ ID | |
|---|---|---|---|---|---|
| NO. | Sequence | NO. | Sequence | NO. | Sequence |
| 1 | GTAATGCCTAGCAGTG | 4 | CAAGCTGAATAGGACC | 7 | CTATCTAGTGAACACC |
| 2 | CGAATCCAGACCCAAG | 5 | CTTAAGGGTGCTGGTC | 8 | GGGTCGAAGTCTGAAC |
| 3 | GGCATACTCAATAGCT | 6 | GCATCTGTTGTGAGTC | 9 | GTAGTCCTGGTTTAGA |

TABLE 2
Sequences of ASO candidates targeting PUFIN3.

| SEQ ID | | SEQ ID | | SEQ ID | |
|---|---|---|---|---|---|
| NO. | Sequence | NO. | Sequence | NO. | Sequence |
| 10 | GTCAGATCCTTGGTAT | 13 | GGTAGTGTTTAGGTAC | 16 | GCACTAATTAACCCTT |
| 11 | GATCATTGTAGCAGAC | 14 | CCATTAGTAGACCATG | | |
| 12 | GTGTCAAGGGTAAGTC | 15 | CTATCTTAGACCCAGC | | |

TABLE 3
Sequences of ASO candidates targeting PUFIN6.

| SEQ ID | | SEQ ID | | SEQ ID | |
|---|---|---|---|---|---|
| NO. | Sequence | NO. | Sequence | NO. | Sequence |
| 17 | TTATCATCAGTCTAGG | 21 | GAATGTAGCTCCTTAG | 25 | CAAACCCGCCATCCAC |
| 18 | CTCCGACTGTGCAATG | 22 | GGATCAACTACAGGGC | 26 | GTCTGATCCTAGCACC |
| 19 | GCAGCATATAACTCTC | 23 | CCACTGAGTATAACTC | | |
| 20 | ATGACAGACCTCGTGT | 24 | AGACTCTAAAGCTCCG | | |

TABLE 4
Sequences of ASO candidates targeting PUFIN7.

| SEQ ID | | SEQ ID | | SEQ ID | |
|---|---|---|---|---|---|
| NO. | Sequence | NO. | Sequence | NO. | Sequence |
| 27 | AGCATAACTCAGCCGT | 30 | GTCATTGTATAGCCAG | 33 | GAAGATTACCTAGCAG |
| 28 | TCTTACGTGTCTGAAG | 31 | GACCCTAGCACTTGAG | 34 | GCAGTACCAGAACTAT |
| 29 | TTACCTTGAGTGACCC | 32 | TGAAGTAGCCATAGCC | 35 | GTCACCACATGTTAGT |

TABLE 5
Sequences of ASO candidates targeting PUFIN1.

| SEQ ID | | SEQ ID | | SEQ ID | |
|---|---|---|---|---|---|
| NO. | Sequence | NO. | Sequence | NO. | Sequence |
| 36 | TCAGATAGAGTTAGAC | 38 | ACAGATAAGTGAACGA | 40 | CGTAGAAGAGGTCGGT |
| 37 | CTCAGTCTGGTCGTGC | 39 | GATAGAGTTAGACATA | | |

TABLE 6
Additional sequences of ASO targeting PUFIN1.

| SEQ | | SEQ | | SEQ | |
|---|---|---|---|---|---|
| ID | | ID | | ID | |
| NO. | Sequence | NO. | Sequence | NO. | Sequence |
| 1406 | GTAAATTGAGCCATAC | 1595 | TCCATGACACTGTAAACTCC | 1784 | TTTAGTTGCCATTGTCACTG |
| 1407 | GTCATGTAAATTGAGC | 1596 | CTCCATGACACTGTAAACTC | 1785 | CTATCATCAGGATCAAACAA |
| 1408 | GTTGCACAATGGTCAC | 1597 | TCTCCATGACACTGTAAACT | 1786 | GCTATCATCAGGATCAAACA |
| 1409 | CAGTTGCACAATGGTC | 1598 | GTTCTGTATTTGGTAGTCTC | 1787 | CAAGCTATCATCAGGATCAA |
| 1410 | TTGGACCCTTGTGATG | 1599 | TAGTTCTGTATTTGGTAGTC | 1788 | CACAAGCTATCATCAGGATC |
| 1411 | GATTAGTTCATAGGGA | 1600 | CATAGTTCTGTATTTGGTAG | 1789 | TCACAAGCTATCATCAGGAT |
| 1412 | GGTCTGTTGATACTGT | 1601 | GCATAGTTCTGTATTTGGTA | 1790 | CTCACAAGCTATCATCAGGA |
| 1413 | GGGTCTGTTGATACTG | 1602 | TGCATAGTTCTGTATTTGGT | 1791 | CACTCACAAGCTATCATCAG |
| 1414 | TGGGTCTGTTGATACT | 1603 | TTGCATAGTTCTGTATTTGG | 1792 | TCACTCACAAGCTATCATCA |
| 1415 | GGATAAGTTTCAACCA | 1604 | GATGTTTGCATAGTTCTGTA | 1793 | CTCACTCACAAGCTATCATC |
| 1416 | GTATTTGGTAGTCTCC | 1605 | GGAATTAGATGTTTGCATAG | 1794 | CCTCACTCACAAGCTATCAT |
| 1417 | GCACTTTCAATCAGCC | 1606 | CAATGGAATTAGATGTTTGC | 1795 | GCACTATCCTTTACTGGCTG |
| 1418 | GGGCAAGCAGTTGATT | 1607 | CATCACAATGGAATTAGATG | 1796 | GGCACTATCCTTTACTGGCT |
| 1419 | GAGCCAATCATAGTAC | 1608 | CTCATCACAATGGAATTAGA | 1797 | TCACCAAACCCAGCCAGAGC |
| 1420 | TGTTAGGAAGGTCACC | 1609 | CCTCATCACAATGGAATTAG | 1798 | GTCACCAAACCCAGCCAGAG |
| 1421 | GGACAACTTGGCTTGA | 1610 | CCCTCATCACAATGGAATTA | 1799 | TGTCACCAAACCCAGCCAGA |
| 1422 | TATTGGACAACTTGGC | 1611 | TAAACATCACACTCATGGAT | 1800 | TTGTCACCAAACCCAGCCAG |
| 1423 | CTGACAAGTGATGCCC | 1612 | CTAAACATCACACTCATGGA | 1801 | CATTGTCACCAAACCCAGCC |
| 1424 | GCTTAGTGTTCATGTC | 1613 | CCTAAACATCACACTCATGG | 1802 | CCATTGTCACCAAACCCAGC |
| 1425 | CCAACTACAAGGGACC | 1614 | GCCTAAACATCACACTCATG | 1803 | GCCATTGTCACCAAACCCAG |
| 1426 | GCCAACTACAAGGGAC | 1615 | CAGCCTAAACATCACACTCA | 1804 | GAGCCATTGTCACCAAACCC |
| 1427 | GTACCTCCAATACACC | 1616 | TCAGCCTAAACATCACACTC | 1805 | GAAAGAGCCATTGTCACCAA |
| 1428 | TGTACCTCCAATACAC | 1617 | CTCAGCCTAAACATCACACT | 1806 | CCAGAGAAAGAGCCATTGTC |
| 1429 | TCAGATAGAGTTAGAC | 1618 | TCTCAGCCTAAACATCACAC | 1807 | TCCAGAGAAAGAGCCATTGT |
| 1430 | GGGTAATATGGGTCAT | 1619 | CTCTCAGCCTAAACATCACA | 1808 | GTCCAGAGAAAGAGCCATTG |
| 1431 | TTAGAGGTAACTGTCC | 1620 | TACTCTCAGCCTAAACATCA | 1809 | TGTCCAGAGAAAGAGCCATT |
| 1432 | GGTTGTGGTGTCATTC | 1621 | CTACTCTCAGCCTAAACATC | 1810 | CTGTCCAGAGAAAGAGCCAT |
| 1433 | TCCCATAGTGACCTAA | 1622 | TCTACTCTCAGCCTAAACAT | 1811 | CAACTGTCCAGAGAAAGAGC |
| 1434 | CTCCCATAGTGACCTA | 1623 | GTCTACTCTCAGCCTAAACA | 1812 | CACAACTGTCCAGAGAAAGA |
| 1435 | GCACTTCACTTTAGGG | 1624 | TGTCTACTCTCAGCCTAAAC | 1813 | CCACAACTGTCCAGAGAAAG |
| 1436 | GTCAGTTACCAATGTC | 1625 | GTGTCTACTCTCAGCCTAAA | 1814 | CCCACAACTGTCCAGAGAAA |
| 1437 | GGATAGTCAGTTACCA | 1626 | TCAGGTGTCTACTCTCAGCC | 1815 | CACCCACAACTGTCCAGAGA |
| 1438 | GGGATAGTCAGTTACC | 1627 | CTCAGGTGTCTACTCTCAGC | 1816 | GCACCCACAACTGTCCAGAG |
| 1439 | TGGGATAGTCAGTTAC | 1628 | GACACAGTCCATTCCCTGCC | 1817 | TGCACCCACAACTGTCCAGA |
| 1440 | TTGGGCATTACTTACC | 1629 | CACAGACACAGTCCATTCCC | 1818 | TCAAAGCCACAATGAACAGG |
| 1441 | GTTGGGCATTACTTAC | 1630 | CACACAGACACAGTCCATTC | 1819 | CTCAAAGCCACAATGAACAG |
| 1442 | CTAGGATTAGAGAGCC | 1631 | CACACACAGACACAGTCCAT | 1820 | GCTCAAAGCCACAATGAACA |
| 1443 | TTGGTGTAGGATATGC | 1632 | TCCCAGACAGATAAGTGAAC | 1821 | CCCAAAGAAACCCATTCTGG |
| 1444 | CTTGGTGTAGGATATG | 1633 | TTCCCAGACAGATAAGTGAA | 1822 | TCCCAAAGAAACCCATTCTG |
| 1445 | GAGACTCCTATGCCCT | 1634 | GTTCCCAGACAGATAAGTGA | 1823 | CCACCAGCTCCCAAAGAAAC |
| 1446 | GGTCACAGCTATATGC | 1635 | CACTTTCAATCAGCCTCTGG | 1824 | GACCACCAGCTCCCAAAGAA |
| 1447 | GCCTAATCTCAGTGGA | 1636 | GCACTTTCAATCAGCCTCTG | 1825 | CTGAAGATATAAACAGCTAC |
| 1448 | TAGAACCTAGAGTGTC | 1637 | CAGCACTTTCAATCAGCCTC | 1826 | TCAACCAAACCCATGCTGTG |
| 1449 | GTAGAACCTAGAGTGT | 1638 | CCAGCACTTTCAATCAGCCT | 1827 | TTCAACCAAACCCATGCTGT |
| 1450 | TGTAGAACCTAGAGTG | 1639 | TCCAGCACTTTCAATCAGCC | 1828 | CTTCAACCAAACCCATGCTG |
| 1451 | GCACTTGTTGGAACTC | 1640 | TTCCAGCACTTTCAATCAGC | 1829 | CCTTCAACCAAACCCATGCT |
| 1452 | GGAAGGTTATACATGC | 1641 | CTTCCAGCACTTTCAATCAG | 1830 | TGTTCATGTCAAGTCCAGGA |
| 1453 | GAGGTGATCCAATGAC | 1642 | GGTCTCCCAGTTGTTCATGC | 1831 | GTGTTCATGTCAAGTCCAGG |
| 1454 | GAATTGACACACTAGG | 1643 | CCAAGGTCTCCCAGTTGTTC | 1832 | TAGTGTTCATGTCAAGTCCA |
| 1455 | GCTTGGATCATTGTGA | 1644 | CCCAAGGTCTCCCAGTTGTT | 1833 | TTAGTGTTCATGTCAAGTCC |
| 1456 | GAGCATTCAAGTAGAC | 1645 | CCACAGAACAGTCACATGGG | 1834 | CTTAGTGTTCATGTCAAGTC |
| 1457 | GAGTGGCAACAGGTCC | 1646 | GCCACAGAACAGTCACATGG | 1835 | GCTTAGTGTTCATGTCAAGT |
| 1458 | CATGTAAATTGAGCCATACA | 1647 | CATCAGAGCCACAGAACAGT | 1836 | CCCAGACAAGACTAAACAGA |
| 1459 | TCATGTAAATTGAGCCATAC | 1648 | TCACATCAGAGCCACAGAAC | 1837 | GCCCAGACAAGACTAAACAG |
| 1460 | GTCATGTAAATTGAGCCATA | 1649 | GTCACATCAGAGCCACAGAA | 1838 | CTTGAAAGTCTGAAGGTGGG |
| 1461 | TGTCATGTAAATTGAGCCAT | 1650 | CAGTCACATCAGAGCCACAG | 1839 | TCTTGAAAGTCTGAAGGTGG |
| 1462 | CTAAGCAACCACTTATCTGC | 1651 | GCAGTCACATCAGAGCCACA | 1840 | CTCTTGAAAGTCTGAAGGTG |
| 1463 | CCTAAGCAACCACTTATCTG | 1652 | GAGCAGTCACATCAGAGCCA | 1841 | GCTCTTGAAAGTCTGAAGGT |
| 1464 | CATGCTATTCAATTCACCCA | 1653 | CAGAGCAGTCACATCAGAGC | 1842 | GCAGCCAACTACAAGGGACC |
| 1465 | CACATGCTATTCAATTCACC | 1654 | CACAGAGCAGTCACATCAGA | 1843 | CTGACTGTATTTGAATTCCA |
| 1466 | CTGTTGTAATGGCTTTATTG | 1655 | CCACAGAGCAGTCACATCAG | 1844 | GCTGACTGTATTTGAATTCC |
| 1467 | GATTCTTTCTGTTGTAATGG | 1656 | GCCACAGAGCAGTCACATCA | 1845 | GAGCTGACTGTATTTGAATT |
| 1468 | GACTGTGACCCTCCCTGCTA | 1657 | GAGCCACAGAGCAGTCACAT | 1846 | GAAGAGCTGACTGTATTTGA |
| 1469 | TTAAGTGACAACTGGTGGGA | 1658 | CAGAGCCACAGAGCAGTCAC | 1847 | CAAGAAGAGCTGACTGTATT |
| 1470 | GATTAAGTGACAACTGGTGG | 1659 | TCAGAGCCACAGAGCAGTCA | 1848 | TCAAGAAGAGCTGACTGTAT |
| 1471 | GAGATTAAGTGACAACTGGT | 1660 | CATCAGAGCCACAGAGCAGT | 1849 | CTCAAGAAGAGCTGACTGTA |
| 1472 | TGAGATTAAGTGACAACTGG | 1661 | CACATCAGAGCCACAGAGCA | 1850 | TACTGTACCTCCAATACACC |
| 1473 | CTGAGATTAAGTGACAACTG | 1662 | TCACATCAGAGCCACAGAGC | 1851 | TTACTGTACCTCCAATACAC |
| 1474 | CCTGAGATTAAGTGACAACT | 1663 | GTCACATCAGAGCCACAGAG | 1852 | CATTACTGTACCTCCAATAC |
| 1475 | TCCTGAGATTAAGTGACAAC | 1664 | GAGTCACATCAGAGCCACAG | 1853 | TCATTACTGTACCTCCAATA |
| 1476 | TTCCTGAGATTAAGTGACAA | 1665 | GAAGAGTCACATCAGAGCCA | 1854 | CATCATTACTGTACCTCCAA |
| 1477 | CTTCCTGAGATTAAGTGACA | 1666 | CAGAAGAGTCACATCAGAGC | 1855 | CACACATCATTACTGTACCT |
| 1478 | TCTTCCTGAGATTAAGTGAC | 1667 | CACAGAAGAGTCACATCAGA | 1856 | CAAACACACATCATTACTGT |
| 1479 | CAGCACAGCTCAGACCTTTG | 1668 | CCACAGAAGAGTCACATCAG | 1857 | GACATTCAAACACACATCAT |
| 1480 | CCAGCACAGCTCAGACCTTT | 1669 | GCCACAGAAGAGTCACATCA | 1858 | TGACATTCAAACACACATCA |
| 1481 | GCCAGCACAGCTCAGACCTT | 1670 | CAGACACATAAGAGCCACAG | 1859 | GTGACATTCAAACACACATC |
| 1482 | TTCAGTTGCACAATGGTCAC | 1671 | GCCACAGAACAGACACATAA | 1860 | GGTGACATTCAAACACACAT |
| 1483 | GTTCAGTTGCACAATGGTCA | 1672 | GAGCCACAGAACAGACACAT | 1861 | TTAGACATACAGAACCACTG |
| 1484 | GAGTCTGTTTATATTCCTGC | 1673 | CAGAGCCACAGAACAGACAC | 1862 | GTTAGACATACAGAACCACT |
| 1485 | GATGCTGAAAGGTGGAGTCT | 1674 | TCAGAGCCACAGAACAGACA | 1863 | GAGTTAGACATACAGAACCA |
| 1486 | CCATGTTCCTAACCAAGCTC | 1675 | TTCAGAGCCACAGAACAGAC | 1864 | TAGAGTTAGACATACAGAAC |
| 1487 | GCCATGTTCCTAACCAAGCT | 1676 | CTTCAGAGCCACAGAACAGA | 1865 | CAGATAGAGTTAGACATACA |
| 1488 | CTGTCAAGCCATGTTCCTAA | 1677 | CACTTCAGAGCCACAGAACA | 1866 | TCAGATAGAGTTAGACATAC |
| 1489 | TGATGGATGGAGTTTAACAT | 1678 | TCACTTCAGAGCCACAGAAC | 1867 | GCCCATGCACACCCTCTTGG |
| 1490 | GTGATGGATGGAGTTTAACA | 1679 | GTCACTTCAGAGCCACAGAA | 1868 | CATGAAGCTGTTAGAGGTAA |
| 1491 | AGTGATGGATGGAGTTTAAC | 1680 | GAGTCACTTCAGAGCCACAG | 1869 | GTGGTGTCATTCAGTCATGA |
| 1492 | TCTAAGACCCAGAGTACATG | 1681 | CACAGAAGAGTTGCATTGGA | 1870 | TGTGGTGTCATTCAGTCATG |
| 1493 | TCCATTCTCTAAGACCCAGA | 1682 | CCACAGAAGAGTTGCATTGG | 1871 | TTGTGGTGTCATTCAGTCAT |
| 1494 | TTCCATTCTCTAAGACCCAG | 1683 | CAGAACAGTCACATCAGGGC | 1872 | GGTTGTGGTGTCATTCAGTC |
| 1495 | GACTTCTCATTTATCCTAAG | 1684 | GCAGAACAGTCACATCAGGG | 1873 | CAGTGGTTGTGGTGTCATTC |
| 1496 | GGCAATACACTCTGGAAGGC | 1685 | CAGAACAGTCTCATAGAGCT | 1874 | GCCTCTAATTCTAGTTGATA |
| 1497 | GATTCAGGCAATACACTCTG | 1686 | CACAGAACAGTCTCATAGAG | 1875 | TTAAGTTCAACATGGAAAGC |
| 1498 | GGATTCAGGCAATACACTCT | 1687 | CCACAGAACAGTCTCATAGA | 1876 | GCCTCAGCACTCTTTATAGG |
| 1499 | CAGGATTCAGGCAATACACT | 1688 | GCCACAGAACAGTCTCATAG | 1877 | CAGCCTCAGCACTCTTTATA |
| 1500 | CAGTCATTTGTGTGCTTGTT | 1689 | CAGAGCCACAGAACAGTCTC | 1878 | TATTAGTTCCCAAGAAAGCT |
| 1501 | GAACAGTCATTTGTGTGCTT | 1690 | TCAGAGCCACAGAACAGTCT | 1879 | CTATTAGTTCCCAAGAAAGC |
| 1502 | TGAACAGTCATTTGTGTGCT | 1691 | CATCAGAGCCACAGAACAGT | 1880 | TCTATTAGTTCCCAAGAAAG |
| 1503 | CAGTCTACCACAGAATGAAC | 1692 | TCACATCAGAGCCACAGAAC | 1881 | CTCTATTAGTTCCCAAGAAA |
| 1504 | TATTCAGTCTACCACAGAAT | 1693 | GTCACATCAGAGCCACAGAA | 1882 | TACATAGCAAGACCTCCCTC |
| 1505 | TTATTCAGTCTACCACAGAA | 1694 | GAGTCACATCAGAGCCACAG | 1883 | CTACATAGCAAGACCTCCCT |
| 1506 | TTTATTCAGTCTACCACAGA | 1695 | GAAGAGTCACATCAGAGCCA | 1884 | CAGATCCTTTAAGAAACAAG |
| 1507 | CTTTATTCAGTCTACCACAG | 1696 | CAGAAGAGTCACATCAGAGC | 1885 | CCAGATCCTTTAAGAAACAA |
| 1508 | CAAGATCCTTCACTTAGTCA | 1697 | TGCAGAAGAGTCACATCAGA | 1886 | TCCAGATCCTTTAAGAAACA |
| 1509 | TCAAGATCCTTCACTTAGTC | 1698 | GGAACAGTTACATCAGAGCT | 1887 | GTCCAGATCCTTTAAGAAAC |
| 1510 | GCTCAAGATCCTTCACTTAG | 1699 | GTGGCTCTGAAGAATGTGGG | 1888 | TCCCATAGTGACCTAAAGTC |
| 1511 | TCCAGCTCAAGATCCTTCAC | 1700 | TGTGGCTCTGAAGAATGTGG | 1889 | CTCCCATAGTGACCTAAAGT |
| 1512 | CTCCAGCTCAAGATCCTTCA | 1701 | GTGTGGCTCTGAAGAATGTG | 1890 | GACTCCCATAGTGACCTAAA |
| 1513 | CAGCCAGCCTCTTCAAACTT | 1702 | CAAAGCTTCCATCTGAGTGT | 1891 | TGATTTAAGCAGAGGTGGAC |
| 1514 | GCAGCCAGCCTCTTCAAACT | 1703 | GCAAAGCTTCCATCTGAGTG | 1892 | TGTGATTTAAGCAGAGGTGG |
| 1515 | GAGAAATGAACATCTGTTGT | 1704 | CACACAGCAAAGCTTCCATC | 1893 | GTGTGATTTAAGCAGAGGTG |
| 1516 | TGAGAAATGAACATCTGTTG | 1705 | GCACACAGCAAAGCTTCCAT | 1894 | CTGCTCTCAAACTTGCTGGG |
| 1517 | GTGAGAAATGAACATCTGTT | 1706 | GATGAATGACAGTTGGTTTC | 1895 | GCTGCTCTCAAACTTGCTGG |
| 1518 | TGTGAGAAATGAACATCTGT | 1707 | TCTTCAGGAAAGACAGGCAG | 1896 | TGCTGCTCTCAAACTTGCTG |
| 1519 | CTGTGAGAAATGAACATCTG | 1708 | GAGGGAGCTGGGACATTTAT | 1897 | GTGCTGCTCTCAAACTTGCT |
| 1520 | CAGCTATAAACCAGGAAGTG | 1709 | TGATCTCAGGAAGCACAGTG | 1898 | GGCTGCACTTCACTTTAGGG |
| 1521 | CCAGCTATAAACCAGGAAGT | 1710 | GCCAATCATAGTACATACAA | 1899 | TGGCTGCACTTCACTTTAGG |
| 1522 | GCCAGCTATAAACCAGGAAG | 1711 | GAGCCAATCATAGTACATAC | 1900 | CTGGCTGCACTTCACTTTAG |
| 1523 | TGCCAGCTATAAACCAGGAA | 1712 | CAGAGCCAATCATAGTACAT | 1901 | TTGAATAACTACCTTGGCTG |
| 1524 | GATGCCAGCTATAAACCAGG | 1713 | GACAGAGCCAATCATAGTAC | 1902 | TTTGAATAACTACCTTGGCT |
| 1525 | GAAGATGCCAGCTATAAACC | 1714 | TGACAGAGCCAATCATAGTA | 1903 | TTCCTGAGCAAGCCATCTTG |
| 1526 | TGAGAAGATGCCAGCTATAA | 1715 | CTGACAGAGCCAATCATAGT | 1904 | CTTCCTGAGCAAGCCATCTT |
| 1527 | GTGAGAAGATGCCAGCTATA | 1716 | TAAACTGACAGAGCCAATCA | 1905 | TCTTCCTGAGCAAGCCATCT |
| 1528 | CAGTGAGAAGATGCCAGCTA | 1717 | TTAAACTGACAGAGCCAATC | 1906 | TTCTTCCTGAGCAAGCCATC |
| 1529 | CACAGTGAGAAGATGCCAGC | 1718 | CATTAAACTGACAGAGCCAA | 1907 | TTTCTTCCTGAGCAAGCCAT |
| 1530 | GGACCCTAGAAAGAGACTTT | 1719 | CCATTAAACTGACAGAGCCA | 1908 | TGCATTTAGAGACACAGAGA |
| 1531 | TGATTAGTTCATAGGGATGA | 1720 | TCCATTAAACTGACAGAGCC | 1909 | TTGCATTTAGAGACACAGAG |
| 1532 | GTGTGTCCCACCCAAATTCA | 1721 | TTCCATTAAACTGACAGAGC | 1910 | TTTGCATTTAGAGACACAGA |
| 1533 | TATTACCTGCTACAGACTGA | 1722 | TTTCCATTAAACTGACAGAG | 1911 | CTTTGCATTTAGAGACACAG |
| 1534 | TTATTACCTGCTACAGACTG | 1723 | TAATGCACCATTACCAACTT | 1912 | GTTGAAATGTCCCATGTTTG |
| 1535 | CTTATTACCTGCTACAGACT | 1724 | CCAAGCATTATTCCTAAGAT | 1913 | TGTTGAAATGTCCCATGTTT |
| 1536 | GCTTATTACCTGCTACAGAC | 1725 | CACCAAGCATTATTCCTAAG | 1914 | CTGTTGAAATGTCCCATGTT |
| 1537 | TGCTTATTACCTGCTACAGA | 1726 | TCACCAAGCATTATTCCTAA | 1915 | TCTGTTGAAATGTCCCATGT |
| 1538 | TATGCTTATTACCTGCTACA | 1727 | TTCACCAAGCATTATTCCTA | 1916 | GAATCTGTTGAAATGTCCCA |
| 1539 | CATATGCTTATTACCTGCTA | 1728 | GTTTCACCAAGCATTATTCC | 1917 | TGAATCTGTTGAAATGTCCC |
| 1540 | GACATATGCTTATTACCTGC | 1729 | TGTTTCACCAAGCATTATTC | 1918 | TTGAATCTGTTGAAATGTCC |
| 1541 | CTGTAACAAAGCACACAAGC | 1730 | GGCAAATTAATTGAGACACA | 1919 | CTTGAATCTGTTGAAATGTC |
| 1542 | TTGTGAAACCTTGCCTGTTT | 1731 | GACACCTGCTTTCTGACATC | 1920 | GCTTGAATCTGTTGAAATGT |
| 1543 | CTTGTGAAACCTTGCCTGTT | 1732 | TAGACACCTGCTTTCTGACA | 1921 | GGCTTGAATCTGTTGAAATG |
| 1544 | TCTTGTGAAACCTTGCCTGT | 1733 | TTAGACACCTGCTTTCTGAC | 1922 | TGGCTTGAATCTGTTGAAAT |
| 1545 | TTCTTGTGAAACCTTGCCTG | 1734 | TTTAGACACCTGCTTTCTGA | 1923 | TTGGCTTGAATCTGTTGAAA |
| 1546 | CAGCTTCCTTTCTTGTGAAA | 1735 | TCTTAGAAGGAAGAGTGACT | 1924 | CCTTGGCTTGAATCTGTTGA |
| 1547 | CAGTGTCCTCCAGAACTAGT | 1736 | GACAACTTGGCTTGATTCTG | 1925 | TCCTTGGCTTGAATCTGTTG |
| 1548 | TTGATACTGTGGGAAGGACA | 1737 | GGACAACTTGGCTTGATTCT | 1926 | TAAAGACTATGATTCTCCCA |
| 1549 | GTTGATACTGTGGGAAGGAC | 1738 | TGGACAACTTGGCTTGATTC | 1927 | TTAAAGACTATGATTCTCCC |
| 1550 | TGTTGATACTGTGGGAAGGA | 1739 | TTGGACAACTTGGCTTGATT | 1928 | GTCTAAATCCTGCCTTATTG |
| 1551 | GTCTGTTGATACTGTGGGAA | 1740 | TATTGGACAACTTGGCTTGA | 1929 | CAATGTCTAAATCCTGCCTT |
| 1552 | GGTCTGTTGATACTGTGGGA | 1741 | TCCTCAGGCAGCACAGATGA | 1930 | CCAATGTCTAAATCCTGCCT |
| 1553 | GGGTCTGTTGATACTGTGGG | 1742 | CATCCTCAGGCAGCACAGAT | 1931 | TACCAATGTCTAAATCCTGC |
| 1554 | TGGGTCTGTTGATACTGTGG | 1743 | CACATCCTCAGGCAGCACAG | 1932 | TTACCAATGTCTAAATCCTG |
| 1555 | CTGGGTCTGTTGATACTGTG | 1744 | TCACATCCTCAGGCAGCACA | 1933 | GTTACCAATGTCTAAATCCT |
| 1556 | TTCAACCAGAGAGTCAGAGA | 1745 | GTCACATCCTCAGGCAGCAC | 1934 | CAGTTACCAATGTCTAAATC |
| 1557 | TTTCAACCAGAGAGTCAGAG | 1746 | GTCCTCAGAGTCCTGCTGCC | 1935 | GTCAGTTACCAATGTCTAAA |
| 1558 | GTTTCAACCAGAGAGTCAGA | 1747 | CAAGTTCCAAGCACTGAGAC | 1936 | GATAGTCAGTTACCAATGTC |
| 1559 | GATAAGTTTCAACCAGAGAG | 1748 | GCAAGTTCCAAGCACTGAGA | 1937 | GGATAGTCAGTTACCAATGT |
| 1560 | GGATAAGTTTCAACCAGAGA | 1749 | CAAGCAAGTTCCAAGCACTG | 1938 | GGGATAGTCAGTTACCAATG |
| 1561 | TGGATAAGTTTCAACCAGAG | 1750 | CCAAGCAAGTTCCAAGCACT | 1939 | TAAACATGGGATAGTCAGTT |
| 1562 | CACAAGAATTGGGAAACTCC | 1751 | CTTAAACCAAGCAAGTTCCA | 1940 | CAAATAAACATGGGATAGTC |
| 1563 | CACACAAGAATTGGGAAACT | 1752 | TCTTAAACCAAGCAAGTTCC | 1941 | GTACTTCAAATAAACATGGG |
| 1564 | CACACACACAAGAATTGGGA | 1753 | TTCTTAAACCAAGCAAGTTC | 1942 | GGTACTTCAAATAAACATGG |
| 1565 | CCACACACACAAGAATTGGG | 1754 | CTTCTTAAACCAAGCAAGTT | 1943 | TTTCACACTGAGACAGGTTT |
| 1566 | CCCACACACACAAGAATTGG | 1755 | GACTTCTTAAACCAAGCAAG | 1944 | TTGGACTAGAGATGCTTAAG |
| 1567 | GACAACTTGCAGAGATAAGC | 1756 | TGACTTCTTAAACCAAGCAA | 1945 | TTTGGACTAGAGATGCTTAA |
| 1568 | CAGAGACAACTTGCAGAGAT | 1757 | CTGACTTCTTAAACCAAGCA | 1946 | GTTTGGACTAGAGATGCTTA |
| 1569 | CCAGAGACAACTTGCAGAGA | 1758 | TCTGACTTCTTAAACCAAGC | 1947 | GGATTCAGCCCAATGATGTC |
| 1570 | TCCAGAGACAACTTGCAGAG | 1759 | TTCTGACTTCTTAAACCAAG | 1948 | CCCTCTAAATTCTCAGATGC |
| 1571 | CATCCAGAGACAACTTGCAG | 1760 | GTTCTGACTTCTTAAACCAA | 1949 | TACCCTCTAAATTCTCAGAT |
| 1572 | GACATCCAGAGACAACTTGC | 1761 | TGTTCTGACTTCTTAAACCA | 1950 | TTACCCTCTAAATTCTCAGA |
| 1573 | GAGACATCCAGAGACAACTT | 1762 | TGATTATCACAGCCCTCCTG | 1951 | CTAATATTCTAGCTTGGTGT |
| 1574 | TGAGACATCCAGAGACAACT | 1763 | GACACCACCAATCCTTGTGC | 1952 | CCTAATATTCTAGCTTGGTG |
| 1575 | GTGAGACATCCAGAGACAAC | 1764 | CAGACACCACCAATCCTTGT | 1953 | GCCTAATATTCTAGCTTGGT |
| 1576 | TGTGAGACATCCAGAGACAA | 1765 | GCAGACACCACCAATCCTTG | 1954 | GTGGTAATGACTTCAGAATC |
| 1577 | GAACAGACTCCAAGCAACTG | 1766 | GGCAGACACCACCAATCCTT | 1955 | GACAGGCACATTACTCAGAA |
| 1578 | TGAACAGACTCCAAGCAACT | 1767 | CAGGCAGACACCACCAATCC | 1956 | CAAGACAGGCACATTACTCA |
| 1579 | CTGCTGAACAGACTCCAAGC | 1768 | TCAGGCAGACACCACCAATC | 1957 | TACAAGACAGGCACATTACT |
| 1580 | GCTGCTGAACAGACTCCAAG | 1769 | TTCAGGCAGACACCACCAAT | 1958 | CTACAAGACAGGCACATTAC |
| 1581 | CTCAGAACATAGATGGCAAA | 1770 | CTTCAGGCAGACACCACCAA | 1959 | TCTACAAGACAGGCACATTA |
| 1582 | CAACTCAGAACATAGATGGC | 1771 | CCTTCAGGCAGACACCACCA | 1960 | CCTAAATAAAGCTGGAGTGA |
| 1583 | TCAACTCAGAACATAGATGG | 1772 | CCCTTCAGGCAGACACCACC | 1961 | GACCTAAATAAAGCTGGAGT |
| 1584 | CATCAACTCAGAACATAGAT | 1773 | CACCCTTCAGGCAGACACCA | 1962 | TGACCTAAATAAAGCTGGAG |
| 1585 | GACATCAACTCAGAACATAG | 1774 | CCACCCTTCAGGCAGACACC | 1963 | TAACAGCCCAGCACCTTGTA |
| 1586 | CAAGACATCAACTCAGAACA | 1775 | GTTAGAATTCCCTGTGCCTC | 1964 | TTAACAGCCCAGCACCTTGT |
| 1587 | TCAAGACATCAACTCAGAAC | 1776 | CATGGTATTAAAGTAGATCC | 1965 | CTTAACAGCCCAGCACCTTG |
| 1588 | TTTCAAAGGAGTGGAGAGCT | 1777 | TTGGAAACACTATTCATGGT | 1966 | CCTTAACAGCCCAGCACCTT |
| 1589 | TAACCACAGAATCCCTAGCC | 1778 | CATTGGAAACACTATTCATG | 1967 | CAACCTGCTCATTGTTACTG |
| 1590 | GAATAACCACAGAATCCCTA | 1779 | GTCATTGGAAACACTATTCA | 1968 | GCAACCTGCTCATTGTTACT |
| 1591 | GGAATAACCACAGAATCCCT | 1780 | TGTCATTGGAAACACTATTC | | |
| 1592 | TGACACTGTAAACTCCTAGT | 1781 | TGCCATTGTCACTGTTAATG | | |
| 1593 | CATGACACTGTAAACTCCTA | 1782 | TTGCCATTGTCACTGTTAAT | | |
| 1594 | CCATGACACTGTAAACTCCT | 1783 | GTTGCCATTGTCACTGTTAA | | |

TABLE 7
Additional sequences of ASO targeting PUFIN7.

| SEQ | | SEQ | | SEQ | |
|---|---|---|---|---|---|
| ID | | ID | | ID | |
| NO. | Sequence | NO. | Sequence | NO. | Sequence |
| 560 | GTTCTCCACAAATATGTCCT | 641 | CTGCTAAGATTGCTACTTTA | 722 | GAACTTACTTGTCTGCTGCT |
| 561 | TGTTCTCCACAAATATGTCC | 642 | TCTGCTAAGATTGCTACTTT | 723 | TGAACTTACTTGTCTGCTGC |
| 562 | CTGTTCTCCACAAATATGTC | 643 | TCATGTAAGGTGGCAGTCTG | 724 | CTGAACTTACTTGTCTGCTG |
| 563 | TCTGTTCTCCACAAATATGT | 644 | GATAGTTCAGTCTTCATTGT | 725 | CTCAAGGGCATGGACACAGC |
| 564 | GTCTGTTCTCCACAAATATG | 645 | CATCTTTCTTAGGTAATGTG | 726 | GTTTCCACTGAACTTGTCTG |
| 565 | CATTGAAGTCTCCAAAGAGC | 646 | CCATCTTTCTTAGGTAATGT | 727 | CAAGATGAAGTTGATGGATC |
| 566 | GAACATTGAAGTCTCCAAAG | 647 | CCCATCTTTCTTAGGTAATG | 728 | CTTAGACAAGATGAAGTTGA |
| 567 | GGAAGGAACATTGAAGTCTC | 648 | GTCATGTGCCAATCTGTCAC | 729 | GCTTAGACAAGATGAAGTTG |
| 568 | TCTGATAAACACTGCACAAA | 649 | CCAGATTATGTCATGTGCCA | 730 | TGCTTAGACAAGATGAAGTT |
| 569 | GTCTGATAAACACTGCACAA | 650 | CCCAGATTATGTCATGTGCC | 731 | CTGCTTAGACAAGATGAAGT |
| 570 | TGTCTGATAAACACTGCACA | 651 | GAACCCAGATTATGTCATGT | 732 | TCTGCTTAGACAAGATGAAG |
| 571 | TCCCAAAGTCTGCCTTCACA | 652 | TAAAGAACCCAGATTATGTC | 733 | CTCTGCTTAGACAAGATGAA |
| 572 | TACACATTACAATTCTGCCC | 653 | GTCTATAAAGAACCCAGATT | 734 | GACTCTGCTTAGACAAGATG |
| 573 | GTACACATTACAATTCTGCC | 654 | GAGTCTATAAAGAACCCAGA | 735 | TGACTCTGCTTAGACAAGAT |
| 574 | TGTACACATTACAATTCTGC | 655 | CAACTGAGTCTATAAAGAAC | 736 | TATGACTCTGCTTAGACAAG |
| 575 | CAACCTGACTTCATTAGACC | 656 | GTTGGTACAAGATAAAGCTA | 737 | CTATGACTCTGCTTAGACAA |
| 576 | GCAACCTGACTTCATTAGAC | 657 | GGTTGGTACAAGATAAAGCT | 738 | GCTATGACTCTGCTTAGACA |
| 577 | GGCAACCTGACTTCATTAGA | 658 | TGGTTGGTACAAGATAAAGC | 739 | TTCAGTCACACTTCTAGCTA |
| 578 | CAAGGCAACCTGACTTCATT | 659 | TGGTGGTTGGTACAAGATAA | 740 | TATTTCAGTCACACTTCTAG |
| 579 | CAACCTGGCATAACACACTT | 660 | CTGGTGGTTGGTACAAGATA | 741 | GTACCAGAACTATTAC |
| 580 | CATCATGTTTCTAGGGTTTC | 661 | CTGGTGGTTGGTACAA | 742 | TGCAGTACCAGAACTATTAC |
| 581 | GCATCATGTTTCTAGGGTTT | 662 | TAAGAGAAAGAGCCTATTCC | 743 | GAGTAGAGGTGACAGTATCT |
| 582 | TGCATCATGTTTCTAGGGTT | 663 | TTAAGAGAAAGAGCCTATTC | 744 | GTAGCCTTAAACTTTATTGG |
| 583 | TTGCATCATGTTTCTAGGGT | 664 | CATTAAGAGAAAGAGCCTAT | 745 | CATGGCAGAATTGTAAACAT |
| 584 | TTTGCATCATGTTTCTAGGG | 665 | TCATTAAGAGAAAGAGCCTA | 746 | GCATGGCAGAATTGTAAACA |
| 585 | CTTTGCATCATGTTTCTAGG | 666 | GTCATTAAGAGAAAGAGCCT | 747 | GGACAAGTATTTACACCTTG |
| 586 | GCTTTGCATCATGTTTCTAG | 667 | TGTTGGAAGTCATTAAGAGA | 748 | TGGACAAGTATTTACACCTT |
| 587 | TCTTAGGCAAGTCCCTTTAG | 668 | CTATGTTGGAAGTCATTAAG | 749 | CTGGACAAGTATTTACACCT |
| 588 | CTCTTATACTATGACAACTC | 669 | GAACTATGTTGGAAGTCATT | 750 | GGTCTCTTTACTGGAC |
| 589 | CCTCTTATACTATGACAACT | 670 | GAGAACTATGTTGGAAGTCA | 751 | GAAGACCTTTAGCCACTCAG |
| 590 | TCCTCTTATACTATGACAAC | 671 | TTGAGAGAACTATGTTGGAA | 752 | GACCTTTAGCCACTCA |
| 591 | CAATCCTCTTATACTATGAC | 672 | TTTGAGAGAACTATGTTGGA | 753 | GAAAGAAGACCTTTAGCCAC |
| 592 | CCAATCCTCTTATACTATGA | 673 | GTTTGAGAGAACTATGTTGG | 754 | TGAAAGAAGACCTTTAGCCA |
| 593 | GAACACTATCAATCCTCCAA | 674 | TCTGGAGGAGTAAGGTTTGA | 755 | TGTGAAAGAAGACCTTTAGC |
| 594 | TAAGAACACTATCAATCCTC | 675 | CTGAAGTAGCCATAGCCAGG | 756 | GATGTGAAAGAAGACCTTTA |
| 595 | GTTAAGAACACTATCAATCC | 676 | TCTGAAGTAGCCATAGCCAG | 757 | TTTGAGGTGGGCAAAGATGT |
| 596 | CTGAAGTTAAGAACACTATC | 677 | TTCTGAAGTAGCCATAGCCA | 758 | TCAGGATTTGAGGTGGGCAA |
| 597 | CCTGAAGTTAAGAACACTAT | 678 | TTTCTGAAGTAGCCATAGCC | 759 | CCAGAGACCTTGCCTTCAGG |
| 598 | CCCTGAAGTTAAGAACACTA | 679 | CTATTATACCAGAGGC | 760 | GAAGACATCCTCAGCCCACC |
| 599 | TCCCTGAAGTTAAGAACACT | 680 | TTCTGCTCTATTATACCAGA | 761 | CTTGACCATATTGCATCCTG |
| 600 | CTCCCTGAAGTTAAGAACAC | 681 | GATTCTGCTCTATTATACCA | 762 | CATCCTTGACCATATTGCAT |
| 601 | TCTCCCTGAAGTTAAGAACA | 682 | TGGTGATTCTGCTCTATTAT | 763 | CCTTGACCATATTGCA |
| 602 | TTCTCCCTGAAGTTAAGAAC | 683 | GAATGTGAGGTGGTGATTCT | 764 | TCATCCTTGACCATATTGCA |
| 603 | GATTCTCCCTGAAGTTAAGA | 684 | TAGAATGTGAGGTGGTGATT | 765 | TACATCTTCAATGCAGACTT |
| 604 | TAGATTCTCCCTGAAGTTAA | 685 | CTTGAAATAGAATGTGAGGT | 766 | GAAATACATCTTCAATGCAG |
| 605 | TTAGATTCTCCCTGAAGTTA | 686 | TGGCTTGAAATAGAATGTGA | 767 | CAGAAATACATCTTCAATGC |
| 606 | CTTAGATTCTCCCTGAAGTT | 687 | TTGGCTTGAAATAGAATGTG | 768 | GCAACAGAAATACATCTTCA |
| 607 | TCTTAGATTCTCCCTGAAGT | 688 | CTTTGGCTTGAAATAGAATG | 769 | TTCATCATTTATAACCAGGG |
| 608 | GTCTTAGATTCTCCCTGAAG | 689 | GACTTTGGCTTGAAATAGAA | 770 | GACCCATTATAGTTTCATCA |
| 609 | GAGACTCCATTCTTCTGAGC | 690 | TGACTTTGGCTTGAAATAGA | 771 | CAGACCCATTATAGTTTCAT |
| 610 | TGAGACTCCATTCTTCTGAG | 691 | TTGACTTTGGCTTGAAATAG | 772 | GACCCATTATAGTTTC |
| 611 | TTGAGACTCCATTCTTCTGA | 692 | GATATTGACTTTGGCTTGAA | 773 | TACAGACCCATTATAGTTTC |
| 612 | TATTTGCCTGATGTCTTGAG | 693 | GAGATATTGACTTTGGCTTG | 774 | CAGACCCATTATAGTT |
| 613 | GTATTTGCCTGATGTCTTGA | 694 | TGAGATATTGACTTTGGCTT | 775 | TACAGACCCATTATAG |
| 614 | GAACTTGGCTCTGATCTCTT | 695 | TTGAGATATTGACTTTGGCT | 776 | CTATTACAGACCCATTATAG |
| 615 | GCTGCCCTTTAGAACTTGGC | 696 | CAGCCTTTGAGATATTGACT | 777 | GCTATTACAGACCCATTATA |
| 616 | TGGTTGACATTTGGGAAATG | 697 | CCAGCCTTTGAGATATTGAC | 778 | GCTATTACAGACCCAT |
| 617 | TTGGTTGACATTTGGGAAAT | 698 | GAACCAGCCTTTGAGATATT | 779 | TACAGACAAGAGCTATATGG |
| 618 | CTTGGTTGACATTTGGGAAA | 699 | CAGAACCAGCCTTTGAGATA | 780 | GTACAGACAAGAGCTATATG |
| 619 | GAAATCATAATTCTCTTGGC | 700 | CACAGAACCAGCCTTTGAGA | 781 | CACATGTTAGTAATGGAAGT |
| 620 | GGAAATCATAATTCTCTTGG | 701 | TCACAGAACCAGCCTTTGAG | 782 | CCACATGTTAGTAATGGAAG |
| 621 | CCAGGAAATCATAATTCTCT | 702 | TAAATCACAGAACCAGCCTT | 783 | CACCACATGTTAGTAATGGA |
| 622 | CTGTCATTGTATAGCC | 703 | TCAGCACTTTCAGTAGGAGC | 784 | TCACCACATGTTAGTAATGG |
| 623 | TTGCACTCCAGCTTTGATCA | 704 | TTCAGCACTTTCAGTAGGAG | 785 | GTCACCACATGTTAGTAATG |
| 624 | TAACTTGCACTCCAGCTTTG | 705 | TTTCAGCACTTTCAGTAGGA | 786 | TGTCACCACATGTTAGTAAT |
| 625 | CTTGAGTACCTTACAC | 706 | CATTTCAGCACTTTCAGTAG | 787 | TTGTCACCACATGTTAGTAA |
| 626 | GACACAGACCCTAGCACTTG | 707 | GACATTTCAGCACTTTCAGT | 788 | CTTGTCACCACATGTTAGTA |
| 627 | GGACACAGACCCTAGCACTT | 708 | GCTATAAGTCTGAAGTGTCA | 789 | GCTTGTCACCACATGTTAGT |
| 628 | TGGACACAGACCCTAGCACT | 709 | GGAGTCTAGGTAGCTA | 790 | TGCTTGTCACCACATGTTAG |
| 629 | TTGGACACAGACCCTAGCAC | 710 | GGCACCCTACACTCTTTGGT | 791 | GAATGCTTGTCACCACATGT |
| 630 | CTTGGACACAGACCCTAGCA | 711 | GAAGGCACCCTACACTCTTT | 792 | GAAGAATGCTTGTCACCACA |
| 631 | GATTGGGCTTGGACACAGAC | 712 | GAAGGCACCCTACACT | 793 | TGAAGAATGCTTGTCACCAC |
| 632 | TGATTGGGCTTGGACACAGA | 713 | GTGAAGGCACCCTACA | 794 | GTAAATGAAGAATGCTTGTC |
| 633 | TTGATTGGGCTTGGACACAG | 714 | CATAGAAGATTACCTAGCAG | 795 | GGTAAATGAAGAATGCTTGT |
| 634 | GATTGGGCTTGGACAC | 715 | GGGCATAGAAGATTACCTAG | 796 | TGGTAAATGAAGAATGCTTG |
| 635 | TGATTGGGCTTGGACA | 716 | TAGGGCATAGAAGATTACCT | 797 | CTGTAAGATAGGGTAAACTA |
| 636 | TTGATTGGGCTTGGAC | 717 | GGCATAGAAGATTACC | 798 | TAACTGGCTGTAAGATAGGG |
| 637 | TTAAGGAAGAGGTGCTGTTG | 718 | TTAGGGCATAGAAGATTACC | 799 | TTTATCTAACTGGCTGTAAG |
| 638 | TTTAAGGAAGAGGTGCTGTT | 719 | CATTAGGGCATAGAAGATTA | 800 | CATTTATCTAACTGGCTGTA |
| 639 | CTAAGATTGCTACTTTAAGG | 720 | CCATTAGGGCATAGAAGATT | 801 | TCATTTATCTAACTGGCTGT |
| 640 | GCTAAGATTGCTACTTTAAG | 721 | CCATTAGGGCATAGAA | | |

TABLE 8
Additional sequences of ASO targeting PUFIN3.

| SEQ | | SEQ | | SEQ | |
|---|---|---|---|---|---|
| ID | | ID | | ID | |
| NO. | Sequence | NO. | Sequence | NO. | Sequence |
| 802 | TCCAGTGACCTCAGTACTCA | 1004 | TGTTGGGTTGGTACACATAG | 1206 | TAATAGTGGTGGTTTAGGGA |
| 803 | TTCCAGTGACCTCAGTACTC | 1005 | GTTGGGTTGGTACACA | 1207 | TTAATAGTGGTGGTTTAGGG |
| 804 | CTTCCAGTGACCTCAGTACT | 1006 | TGTTGGGTTGGTACAC | 1208 | GAGTCCCAAATTTAATAGTG |
| 805 | GCTTCCAGTGACCTCAGTAC | 1007 | GTGTTGGGTTGGTACA | 1209 | CAGAGTCCCAAATTTAATAG |
| 806 | GGCTTCCAGTGACCTCAGTA | 1008 | TATAGTGTTGGGTTGGTACA | 1210 | GTTTCAGAGTCCCAAATTTA |
| 807 | TGGCTTCCAGTGACCTCAGT | 1009 | CTATAGTGTTGGGTTGGTAC | 1211 | GGGTATTTCTGTGGTTTCAG |
| 808 | GAAGATTCCACCAGTTTCCA | 1010 | TATAGTGTTGGGTTGG | 1212 | TAGGGTATTTCTGTGGTTTC |
| 809 | TGAAGATTCCACCAGTTTCC | 1011 | CTATAGTGTTGGGTTG | 1213 | GTAGGGTATTTCTGTGGTTT |
| 810 | CATGAAGATTCCACCAGTTT | 1012 | GATGCTCAAATGATAGTTGT | 1214 | TGTAGGGTATTTCTGTGGTT |
| 811 | CCATGAAGATTCCACCAGTT | 1013 | CATAGATGCTCAAATGATAG | 1215 | TCAGTATTTGAGGAAATCAG |
| 812 | TCCATGAAGATTCCACCAGT | 1014 | GTACATAGATGCTCAAATGA | 1216 | GTCAGTATTTGAGGAAATCA |
| 813 | TTCCATGAAGATTCCACCAG | 1015 | GGTACATAGATGCTCAAATG | 1217 | TGTCAGTATTTGAGGAAATC |
| 814 | TCAGATCCTTGGTATTAGCC | 1016 | TGGTACATAGATGCTCAAAT | 1218 | CTCTCCATTGTTTAGGTTAA |
| 815 | GATCCTTGGTATTAGC | 1017 | TGGTTACATCAGCACCCTGG | 1219 | TTCTTATCTACCAAGTGCTG |
| 816 | GTCAGATCCTTGGTATTAGC | 1018 | CACTCAGCATGGTTACATCA | 1220 | GCATCACTGAAGAATTTGTA |
| 817 | TGTCAGATCCTTGGTATTAG | 1019 | CAGCACTCAGCATGGTTACA | 1221 | CAAGCATCACTGAAGAATTT |
| 818 | TATGTCAGATCCTTGGTATT | 1020 | GTACCTCAGCACTCAGCATG | 1222 | CCAAGCATCACTGAAGAATT |
| 819 | CTATGTCAGATCCTTGGTAT | 1021 | TCAGCATGAGTACCTC | 1223 | GCCAAGCATCACTGAAGAAT |
| 820 | TCTATGTCAGATCCTTGGTA | 1022 | CATAGGCACAATCTTCTCTC | 1224 | GAGCCAAGCATCACTGAAGA |
| 821 | GTCTATGTCAGATCCTTGGT | 1023 | CCATAGGCACAATCTTCTCT | 1225 | TGCAAAGTCAGCCAGCCAGC |
| 822 | TGTCTATGTCAGATCCTTGG | 1024 | TACCATAGGCACAATCTTCT | 1226 | TTGCAAAGTCAGCCAGCCAG |
| 823 | CTATGTCAGATCCTTG | 1025 | CTACCATAGGCACAATCTTC | 1227 | TAGAAGTAGTTGCAAAGTCA |
| 824 | TTGTCTATGTCAGATCCTTG | 1026 | CTCATTCCTGGCTACCATAG | 1228 | CTAGAAGTAGTTGCAAAGTC |
| 825 | GTCTATGTCAGATCCT | 1027 | CAAGAACTTTCTATCTCATC | 1229 | GAACCCTTAACTAGAAGTAG |
| 826 | TTTGTTAAGACCATCTCAGG | 1028 | CTGCCTCTTGCCTAAATTAG | 1230 | TGGAACCCTTAACTAG |
| 827 | CAACTGAACTTTGCCTGCCT | 1029 | GCTGCCTCTTGCCTAAATTA | 1231 | CCTTTGGAACCCTTAACTAG |
| 828 | TAATGTAGAAGGGTCC | 1030 | CTCCAAGGTAATTCAGGACC | 1232 | GCCATCAGATACCACTCATC |
| 829 | TTAGGATCAACATGGG | 1031 | TCTCCAAGGTAATTCAGGAC | 1233 | CAGCCATCAGATACCACTCA |
| 830 | GGCTTGTTAGGATCAA | 1032 | TTCTCCAAGGTAATTCAGGA | 1234 | CCAGCCATCAGATACCACTC |
| 831 | GTGGGACTTGGCTTGT | 1033 | TTTCTCCAAGGTAATTCAGG | 1235 | GCCAGGACCAGCCATCAGAT |
| 832 | CAATGTGGGACTTGGCTTGT | 1034 | GTAAGTCTCATGTTCCTACC | 1236 | TGCCAGGACCAGCCATCAGA |
| 833 | GATGCAATGTGGGACTTGGC | 1035 | GGTAAGTCTCATGTTCCTAC | 1237 | GGTTCAAGTCCTAAACTGGC |
| 834 | TGATGCAATGTGGGACTTGG | 1036 | GTCAAGGGTAAGTCTC | 1238 | GGATTACTCTGACTGGAATC |
| 835 | GATGATGCAATGTGGGACTT | 1037 | CATGTGTCAAGGGTAAGTCT | 1239 | TGGATTACTCTGACTGGAAT |
| 836 | GGATGATGCAATGTGGGACT | 1038 | TCATGTGTCAAGGGTAAGTC | 1240 | TTGGATTACTCTGACTGGAA |
| 837 | TGTGGGATGATGCAATGTGG | 1039 | TAATCATGTGTCAAGGGTAA | 1241 | TTTGGATTACTCTGACTGGA |
| 838 | GAGTTTCACATCTTGAAGTT | 1040 | TAATAATCATGTGTCAAGGG | 1242 | GGATTACTCTGACTGG |
| 839 | GGAGTTTCACATCTTGAAGT | 1041 | TTGCTTGTCAGAAACACAAA | 1243 | GTTTGGATTACTCTGACTGG |
| 840 | GAACCCAATACAGGAGACTC | 1042 | CAAATTGCTTGTCAGAAACA | 1244 | TAGGTTTGGATTACTCTGAC |
| 841 | TGAACCCAATACAGGAGACT | 1043 | CATTTGGCAATTCTGGAGGC | 1245 | CTAGGTTTGGATTACTCTGA |
| 842 | TAATGAACCCAATACAGGAG | 1044 | TCATTTGGCAATTCTGGAGG | 1246 | GACTAGGTTTGGATTACTCT |
| 843 | TTAATGAACCCAATACAGGA | 1045 | GACTCATTTGGCAATTCTGG | 1247 | TAGGTTTGGATTACTC |
| 844 | TCATTGTAGCAGACCAGAAA | 1046 | CAGACTCATTTGGCAATTCT | 1248 | GACTAGGTTTGGATTA |
| 845 | GATCATTGTAGCAGACCAGA | 1047 | GTAACAACCAGACTCATTTG | 1249 | GCAGCCAGAATTACTAAGCT |
| 846 | CATTGTAGCAGACCAG | 1048 | GCAGATGTTACCTTGAGTCT | 1250 | TATTTGGAAACTGGTGAGGC |
| 847 | GGATCATTGTAGCAGA | 1049 | CACTAGCAATAAGCAGATGT | 1251 | TTATTTGGAAACTGGTGAGG |
| 848 | TTTGTGCCTTTGGACTATAG | 1050 | CCTGGTCTATGCACTA | 1252 | GAATTCTTCTCCTAAAGTAC |
| 849 | GTGCCTTTGGACTATA | 1051 | TCTGGGTCTACTTGCAATTT | 1253 | CTGAATTCTTCTCCTAAAGT |
| 850 | CATTAGTACAGGTTTAGGTG | 1052 | GGGTCTACTTGCAATT | 1254 | CCTGAATTCTTCTCCTAAAG |
| 851 | TAGTACAGGTTTAGGT | 1053 | TTCTGGGTCTACTTGCAATT | 1255 | GCCTGAATTCTTCTCCTAAA |
| 852 | TACATTAGTACAGGTTTAGG | 1054 | TGGGTCTACTTGCAAT | 1256 | CCTATCTTAGACCCAGCCTG |
| 853 | GCATCTAGTGGTATAT | 1055 | TAATTCTGGGTCTACTTGCA | 1257 | GCCTATCTTAGACCCAGCCT |
| 854 | TGAAGCATCTAGTGGT | 1056 | TAAACAGTTCCTATTTCTGC | 1258 | GCCTATCTTAGACCCA |
| 855 | TCCCATACTGAAGCATCTAG | 1057 | CTAAACAGTTCCTATTTCTG | 1259 | CAACTTAAGACTTCCTTATG |
| 856 | CTCCCATACTGAAGCATCTA | 1058 | GCTAAACAGTTCCTATTTCT | 1260 | CCAACTTAAGACTTCCTTAT |
| 857 | TCTCCCATACTGAAGCATCT | 1059 | GGCTAAACAGTTCCTATTTC | 1261 | TTATCAAATGCACCCTTAGG |
| 858 | CCCATACTGAAGCATC | 1060 | GGCTAAACAGTTCCTA | 1262 | GTTATCAAATGCACCCTTAG |
| 859 | TATCTCCCATACTGAAGCAT | 1061 | GTTACCATCTAAGCCAGATC | 1263 | TGTTATCAAATGCACCCTTA |
| 860 | CAATATCTCCCATACTGAAG | 1062 | TGTTACCATCTAAGCCAGAT | 1264 | TTGTTATCAAATGCACCCTT |
| 861 | TCAATATCTCCCATACTGAA | 1063 | TTGTTACCATCTAAGCCAGA | 1265 | GTTGTTATCAAATGCACCCT |
| 862 | TTCAATATCTCCCATACTGA | 1064 | TTTGTTACCATCTAAGCCAG | 1266 | GTTATCAAATGCACCC |
| 863 | CAGCACCCTTAGAACTTTAG | 1065 | CTTTGTTACCATCTAAGCCA | 1267 | TGTTGTTATCAAATGCACCC |
| 864 | CACAGCACCCTTAGAACTTT | 1066 | CACTTTGTTACCATCTAAGC | 1268 | TTGTTGTTATCAAATGCACC |
| 865 | CCACAGCACCCTTAGAACTT | 1067 | TCACTTTGTTACCATCTAAG | 1269 | GGTGCTTGTTAAATATTTGC |
| 866 | CACCACAGCACCCTTAGAAC | 1068 | CTGATGGACAGCTACAATCC | 1270 | CTGATAGATAATAGGTGCTT |
| 867 | CCACCACAGCACCCTTAGAA | 1069 | CAGCAGTTTCATCCAGTGTG | 1271 | TAGTGCCTGATAGATAATAG |
| 868 | TCCACCACAGCACCCTTAGA | 1070 | TCAGCAGTTTCATCCAGTGT | 1272 | GTAGTGCCTGATAGATAATA |
| 869 | CTCCACCACAGCACCCTTAG | 1071 | TTCAGCAGTTTCATCCAGTG | 1273 | TGTAGTGCCTGATAGATAAT |
| 870 | CATATCTCCACCACAGCACC | 1072 | CTTCAGCAGTTTCATCCAGT | 1274 | TATGTAGTGCCTGATAGATA |
| 871 | CCATATCTCCACCACAGCAC | 1073 | CCTTCAGCAGTTTCATCCAG | 1275 | GTAGTGCCTGATAGAT |
| 872 | TCCATATCTCCACCACAGCA | 1074 | GCCTTTAGCACCACAGCCTG | 1276 | TGTAGTGCCTGATAGA |
| 873 | TGTCCATATCTCCACCACAG | 1075 | TGCCTTTAGCACCACAGCCT | 1277 | TATATGTAGTGCCTGATAGA |
| 874 | CATCCTTTCTCATAACCAGA | 1076 | GTACAGAATCTTAATGGGCC | 1278 | GTATATGTAGTGCCTGATAG |
| 875 | GGGCATAGTTCTCAGT | 1077 | GGTACAGAATCTTAATGGGC | 1279 | TGTATATGTAGTGCCTGATA |
| 876 | TTCCAGCTACACTCACTTGA | 1078 | TAGGTACAGAATCTTAATGG | 1280 | GAATGTATATGTAGTGCCTG |
| 877 | GACATCTTCCAGCTACACTC | 1079 | GGTGGGTAGTGTTTAGGTAC | 1281 | GAGAATGTATATGTAGTGCC |
| 878 | TGACATCTTCCAGCTACACT | 1080 | GGTGGGTAGTGTTTAG | 1282 | GGAAATAATTGCTAGGATAC |
| 879 | TAACTAGGGTTCTGATGACA | 1081 | CTGAAAGTTGCTAACCATTT | 1283 | CATGACTCCTGCACAAGAAG |
| 880 | CTAGGGTTCTGATGAC | 1082 | TCTGAAAGTTGCTAACCATT | 1284 | TTCCACTAAATAGCCAAGGT |
| 881 | TTAACTAGGGTTCTGATGAC | 1083 | TTCTGAAAGTTGCTAACCAT | 1285 | CAAATTCCACTAAATAGCCA |
| 882 | TATTAACTAGGGTTCTGATG | 1084 | TTTCTGAAAGTTGCTAACCA | 1286 | CCAAATTCCACTAAATAGCC |
| 883 | TAACTAGGGTTCTGAT | 1085 | GTTTCTGAAAGTTGCTAACC | 1287 | CACCAAATTCCACTAAATAG |
| 884 | GTTATTAACTAGGGTTCTGA | 1086 | GTGAAGCAGAGTTTAATGTT | 1288 | GAATCACCAAATTCCACTAA |
| 885 | TGTTATTAACTAGGGTTCTG | 1087 | TGTGAAGCAGAGTTTAATGT | 1289 | TGAATCACCAAATTCCACTA |
| 886 | CATGTTATTAACTAGGGTTC | 1088 | GTGCTGAAAGTTTATGGTCC | 1290 | CTGAATCACCAAATTCCACT |
| 887 | CACATGTTATTAACTAGGGT | 1089 | GGTGCTGAAAGTTTATGGTC | 1291 | TCTGAATCACCAAATTCCAC |
| 888 | GACCACATGTTATTAACTAG | 1090 | CAAGGTGCTGAAAGTTTATG | 1292 | TTCTGAATCACCAAATTCCA |
| 889 | GTAGACCACATGTTATTAAC | 1091 | TCAAGGTGCTGAAAGTTTAT | 1293 | GTTCTGAATCACCAAATTCC |
| 890 | GTAGCAATGGTAGACC | 1092 | TTCAAGGTGCTGAAAGTTTA | 1294 | CATGACAATCTTGGCTTCCC |
| 891 | TGTAGCAATGGTAGAC | 1093 | GTTCAAGGTGCTGAAAGTTT | 1295 | GAACAGCTCCATGACAATCT |
| 892 | GAGGGCATTGTATTGT | 1094 | TGTTCAAGGTGCTGAAAGTT | 1296 | GACATTTGTACTTGAGATAG |
| 893 | CTGTGAATGTAATTTGAGGA | 1095 | TTGTTCAAGGTGCTGAAAGT | 1297 | CCACTCAATAAGAACTTAGT |
| 894 | TCTGTGAATGTAATTTGAGG | 1096 | TTTGTTCAAGGTGCTGAAAG | 1298 | CACCACTCAATAAGAACTTA |
| 895 | TTCATAGAGCATGGGTTGTT | 1097 | CTGTAGAAGGGATTGAATTG | 1299 | CCACCACTCAATAAGAACTT |
| 896 | CTTCATAGAGCATGGGTTGT | 1098 | GGTGAATTACAGGCTA | 1300 | GCCACCACTCAATAAGAACT |
| 897 | CCTTCATAGAGCATGGGTTG | 1099 | TTTAGAAGGTGGTGAATTAC | 1301 | CATGTCCTGAAAGCCTTCCT |
| 898 | TCCTTCATAGAGCATGGGTT | 1100 | CTAAATGTGAGACCTGAGAG | 1302 | GCTTAATAATCAACACAGAC |
| 899 | CTCCTTCATAGAGCATGGGT | 1101 | GCTAAATGTGAGACCTGAGA | 1303 | CAGCTTAATAATCAACACAG |
| 900 | GACTCCTTCATAGAGCATGG | 1102 | TGCTAAATGTGAGACCTGAG | 1304 | GCAACAGCTTAATAATCAAC |
| 901 | GACTCCTTCATAGAGC | 1103 | GATGCTAAATGTGAGACCTG | 1305 | TGGAGTTTACAGGCAACAGC |
| 902 | CATTGGGAAGGATAGTATGC | 1104 | GGATGCTAAATGTGAGACCT | 1306 | CAAAGCTGAAATGTGATTAC |
| 903 | TCATTGGGAAGGATAGTATG | 1105 | TGGATGCTAAATGTGAGACC | 1307 | GCAAAGCTGAAATGTGATTA |
| 904 | GAACTGTCACATCTCCCTCA | 1106 | CTGGATGCTAAATGTGAGAC | 1308 | GGCAAAGCTGAAATGTGATT |
| 905 | GAATCCTGAACTGTCACATC | 1107 | TCTGGATGCTAAATGTGAGA | 1309 | TGGCAAAGCTGAAATGTGAT |
| 906 | TGAATCCTGAACTGTCACAT | 1108 | TTAAGTCCTGAGTTGTCTGG | 1310 | CTGGCAAAGCTGAAATGTGA |
| 907 | CTGAATCCTGAACTGTCACA | 1109 | CTTAAGTCCTGAGTTGTCTG | 1311 | TCTGGCAAAGCTGAAATGTG |
| 908 | CCTGAATCCTGAACTGTCAC | 1110 | GTCATTCCCATACAGAATTT | 1312 | CTCTGGCAAAGCTGAAATGT |
| 909 | TCCTGAATCCTGAACTGTCA | 1111 | TGTCATTCCCATACAGAATT | 1313 | CCTCTGGCAAAGCTGAAATG |
| 910 | TTCCTGAATCCTGAACTGTC | 1112 | CACATCTTTGCTGGTTTGAA | 1314 | CCCTCTGGCAAAGCTGAAAT |
| 911 | TTTCCTGAATCCTGAACTGT | 1113 | CATAATTGCTATTCACTACC | 1315 | TCCCTCTGGCAAAGCTGAAA |
| 912 | CTTTCCTGAATCCTGAACTG | 1114 | GCCCTCAGAACCACAGGTCT | 1316 | GAAATCCCTCTGGCAAAGCT |
| 913 | GTCCTTTCCTGAATCCTGAA | 1115 | CAGCCCTCAGAACCACAGGT | 1317 | TGCTGGGATGATACTGACAG |
| 914 | GGTCCTTTCCTGAATCCTGA | 1116 | GACAGCCCTCAGAACCACAG | 1318 | CTGCTGGGATGATACTGACA |
| 915 | CCATCCTAAAGCACACTTGC | 1117 | GTTGCAAAGGTTTAGC | 1319 | GCTGCTGGGATGATACTGAC |
| 916 | TCCATCCTAAAGCACACTTG | 1118 | TCAGGTTGCAAAGGTTTAGC | 1320 | TAACTGCAAACTGGAGCTAA |
| 917 | CTCCATCCTAAAGCACACTT | 1119 | CTCAGGTTGCAAAGGTTTAG | 1321 | GTGTGTTCTTGTAGTGAGGA |
| 918 | CCTCCATCCTAAAGCACACT | 1120 | GGCTCAGGTTGCAAAGGTTT | 1322 | GAGTGTGTTCTTGTAGTGAG |
| 919 | CCCTCCATCCTAAAGCACAC | 1121 | CAACTTAACTTAGAGCATGG | 1323 | GGAGTGTGTTCTTGTAGTGA |
| 920 | GCCCTCCATCCTAAAGCACA | 1122 | CCAACTTAACTTAGAGCATG | 1324 | GGGAGTGTGTTCTTGTAGTG |
| 921 | TGCCCTCCATCCTAAAGCAC | 1123 | GCCAACTTAACTTAGAGCAT | 1325 | GGGACTCAATATCTGG |
| 922 | CTGCCCTCCATCCTAAAGCA | 1124 | TGCCAACTTAACTTAGAGCA | 1326 | TATAAGGAATTGAAGCACTG |
| 923 | GAAATCTACACTCCTGGCAC | 1125 | CAACTTAACTTAGAGC | 1327 | CCTCAGAATATTGCTCAATA |
| 924 | TGAAATCTACACTCCTGGCA | 1126 | CTGCCAACTTAACTTAGAGC | 1328 | GCCTCAGAATATTGCTCAAT |
| 925 | GATGAAATCTACACTCCTGG | 1127 | GCTGCCAACTTAACTTAGAG | 1329 | TAGCCTCAGAATATTGCTCA |
| 926 | GCAGCCAACAGATGAAATCT | 1128 | GCCAACTTAACTTAGA | 1330 | TATAGCCTCAGAATATTGCT |
| 927 | TGCAGCCAACAGATGAAATC | 1129 | TGCTGCCAACTTAACTTAGA | 1331 | GTATAGCCTCAGAATATTGC |
| 928 | TTGCAGCCAACAGATGAAAT | 1130 | CATGCTGCCAACTTAACTTA | 1332 | CAGTATAGCCTCAGAATATT |
| 929 | TTTGCAGCCAACAGATGAAA | 1131 | CCATGCTGCCAACTTAACTT | 1333 | CACAGTATAGCCTCAGAATA |
| 930 | CACATTTGCAGCCAACAGAT | 1132 | GCCATGCTGCCAACTTAACT | 1334 | TAACACAGTATAGCCTCAGA |
| 931 | GGGAACTGGCACACATTTGC | 1133 | TGCCATGCTGCCAACTTAAC | 1335 | TTAACACAGTATAGCCTCAG |
| 932 | TGGGAACTGGCACACATTTG | 1134 | CTGCCATGCTGCCAACTTAA | 1336 | TTTAACACAGTATAGCCTCA |
| 933 | TTGGGAACTGGCACACATTT | 1135 | GAATCTGTGCTCTCAATCTC | 1337 | GTTGTGATTCTGGAGGCTGC |
| 934 | CATTGGGAACTGGCACACAT | 1136 | TAATTTGAGCCCATGCAGTC | 1338 | TGTTGTGATTCTGGAGGCTG |
| 935 | GAACAGTAGCATTCCATTGG | 1137 | GTAATTTGAGCCCATGCAGT | 1339 | CTGTTGTGATTCTGGAGGCT |
| 936 | GAGAACAGTAGCATTCCATT | 1138 | CTGTAATTTGAGCCCATGCA | 1340 | TCTGTTGTGATTCTGGAGGC |
| 937 | CAGAGAACAGTAGCATTCCA | 1139 | GTGGTAAATTATGAAGCTGT | 1341 | GACTCTCTCTGAATCTGTTG |
| 938 | TCAGAGAACAGTAGCATTCC | 1140 | TGTGGTAAATTATGAAGCTG | 1342 | CAAATGTTGAGCTGTAATCA |
| 939 | GTCAGAGAACAGTAGCATTC | 1141 | CATGTGGTAAATTATGAAGC | 1343 | GCAAATGTTGAGCTGTAATC |
| 940 | GGTCAGAGAACAGTAGCATT | 1142 | TATTTCTTACTGTGGATTGC | 1344 | GCCTGTAACAATAGCTGAGG |
| 941 | GTTATGAGTTGAGGGCAGGT | 1143 | CATAGAATAGGACCAATCAA | 1345 | CATGAGGACAAACTGTAACC |
| 942 | TGTTATGAGTTGAGGGCAGG | 1144 | TAGAATAGGACCAATC | 1346 | CCATGAGGACAAACTGTAAC |
| 943 | GTGTTATGAGTTGAGGGCAG | 1145 | TACATAGAATAGGACCAATC | 1347 | CTAAATTTGAGTCCATGAGG |
| 944 | TGTGTTATGAGTTGAGGGCA | 1146 | TGGATTATGACTAGCA | 1348 | TCTAAATTTGAGTCCATGAG |
| 945 | GTTATGAGTTGAGGGC | 1147 | CAATTTGCTGGATTATGACT | 1349 | CTTCTAAATTTGAGTCCATG |
| 946 | TTGTGTTATGAGTTGAGGGC | 1148 | TCAATTTGCTGGATTATGAC | 1350 | GCCATACTTCTAAATTTGAG |
| 947 | GTGAATTGTGACTTGTGTTA | 1149 | CATGAAATCAATTTGCTGGA | 1351 | GCACTTGTTAAACTGAACTA |
| 948 | CAGTGAATTGTGACTTGTGT | 1150 | GACCATGAAATCAATTTGCT | 1352 | TGCACTTGTTAAACTGAACT |
| 949 | CCAGTGAATTGTGACTTGTG | 1151 | CATTAGTAGACCATGAAATC | 1353 | GTCAGAAGCCATATGCACTT |
| 950 | TGAAACTCCAGTGAATTGTG | 1152 | CCATTAGTAGACCATGAAAT | 1354 | TGTCAGAAGCCATATGCACT |
| 951 | GTGAAACTCCAGTGAATTGT | 1153 | CATTAGTAGACCATGA | 1355 | TTGTCAGAAGCCATATGCAC |
| 952 | TGTGAAACTCCAGTGAATTG | 1154 | GAAACCATTAGTAGACCATG | 1356 | TTTGTCAGAAGCCATATGCA |
| 953 | CTGTGAAACTCCAGTGAATT | 1155 | TGAAACCATTAGTAGACCAT | 1357 | CTTTGTCAGAAGCCATATGC |
| 954 | TCTGATTCCAAACATCCACA | 1156 | CTGAAACCATTAGTAGACCA | 1358 | CCTTTGTCAGAAGCCATATG |
| 955 | TTCTGATTCCAAACATCCAC | 1157 | TCTGAAACCATTAGTAGACC | 1359 | CACCTTTGTCAGAAGCCATA |
| 956 | TTTCTGATTCCAAACATCCA | 1158 | CAAATTGTGGGTCTGAAACC | 1360 | CACACCTTTGTCAGAAGCCA |
| 957 | CATTTCTGATTCCAAACATC | 1159 | TCAAATTGTGGGTCTGAAAC | 1361 | CCACACCTTTGTCAGAAGCC |
| 958 | GCATTTCTGATTCCAAACAT | 1160 | TTCAAATTGTGGGTCTGAAA | 1362 | GCCACACCTTTGTCAGAAGC |
| 959 | TGCATTTCTGATTCCAAACA | 1161 | TTTCAAATTGTGGGTCTGAA | 1363 | GAATACCATGTAAGTGACAG |
| 960 | CTGCATTTCTGATTCCAAAC | 1162 | CCACAGAGAGGATATTATTT | 1364 | TGAATACCATGTAAGTGACA |
| 961 | TCTGCATTTCTGATTCCAAA | 1163 | TTCATAACAACTCCACAGAG | 1365 | CTGAATACCATGTAAGTGAC |
| 962 | GTCTGCATTTCTGATTCCAA | 1164 | GTCATCTTCATAACAACTCC | 1366 | CCTGAATACCATGTAAGTGA |
| 963 | GTGAGTCTGCATTTCTGATT | 1165 | TACCTTCCAAGCTAAACCTT | 1367 | CCCTGAATACCATGTAAGTG |
| 964 | TGTGAGTCTGCATTTCTGAT | 1166 | TTACCTTCCAAGCTAAACCT | 1368 | TCCCTGAATACCATGTAAGT |
| 965 | CTCTTTAAATCCTATACAGC | 1167 | TATTACCTTCCAAGCTAAAC | 1369 | CTCCCTGAATACCATGTAAG |
| 966 | CCAAATTAGTAAGTCTTCAG | 1168 | CTTATTACCTTCCAAGCTAA | 1370 | CCTCCCTGAATACCATGTAA |
| 967 | GCCAAATTAGTAAGTCTTCA | 1169 | GCACTTATTACCTTCCAAGC | 1371 | TCCTCCCTGAATACCATGTA |
| 968 | CTAAACTGCCATGCAACCCT | 1170 | GAGCACTTATTACCTTCCAA | 1372 | GTCCTCCCTGAATACCATGT |
| 969 | GCTAAACTGCCATGCAACCC | 1171 | TGAGCACTTATTACCTTCCA | 1373 | TGTCCTCCCTGAATACCATG |
| 970 | CTCTGCTAAACTGCCATGCA | 1172 | CACTGAGCACTTATTACCTT | 1374 | GTGTCCTCCCTGAATACCAT |
| 971 | TACTCTGCTAAACTGCCATG | 1173 | TCACTGAGCACTTATTACCT | 1375 | TGTGTCCTCCCTGAATACCA |
| 972 | CTACTCTGCTAAACTGCCAT | 1174 | TTCACTGAGCACTTATTACC | 1376 | CTCAATATGATCTCAAAGCT |
| 973 | TTAACTACTCTGCTAAACTG | 1175 | CACTAATATTCACTGAGCAC | 1377 | TCTCAATATGATCTCAAAGC |
| 974 | GTTTAACTACTCTGCTAAAC | 1176 | CCCTTCCTTGAGTAATAATA | 1378 | GAACACTCCTCATATATTAC |
| 975 | CAAGCCCATTTGTTTAACTA | 1177 | GCCCTTCCTTGAGTAATAAT | 1379 | CAGAACACTCCTCATATATT |
| 976 | TCAAGCCCATTTGTTTAACT | 1178 | TGCCCTTCCTTGAGTAATAA | 1380 | CCAGAACACTCCTCATATAT |
| 977 | TGAGTTTCAAGCCCATTTGT | 1179 | GATGCCCTTCCTTGAGTAAT | 1381 | GCCAGAACACTCCTCATATA |
| 978 | CACAATTTAAGATTCTCAGG | 1180 | TGATGCCCTTCCTTGAGTAA | 1382 | TATGCTCACAGTCAAATCTT |
| 979 | GCACACAATTTAAGATTCTC | 1181 | CTGATGCCCTTCCTTGAGTA | 1383 | TTATGCTCACAGTCAAATCT |
| 980 | GGCACACAATTTAAGATTCT | 1182 | CACTGATGCCCTTCCTTGAG | 1384 | GTTATGCTCACAGTCAAATC |
| 981 | CTAAGGCACACAATTTAAGA | 1183 | GATTTATTTCACACTGATGC | 1385 | TGTTATGCTCACAGTCAAAT |
| 982 | CCTAAGGCACACAATTTAAG | 1184 | GGCATGATTACTGGGAGCTT | 1386 | CAATGTTATGCTCACAGTCA |
| 983 | TCTTACAAATAACCTCTGAG | 1185 | GGGCATGATTACTGGGAGCT | 1387 | CATGTCTTGAATGAAGTAAC |
| 984 | GACTTGGCTTTGCATGATAG | 1186 | CATGATTACTGGGAGC | 1388 | CCATGTCTTGAATGAAGTAA |
| 985 | CAGAAGACTTGGCTTTGCAT | 1187 | GGGCATGATTACTGGG | 1389 | TCCATGTCTTGAATGAAGTA |
| 986 | TCAGAAGACTTGGCTTTGCA | 1188 | TGAAGGGCATGATTACTGGG | 1390 | TTCCATGTCTTGAATGAAGT |
| 987 | TATTTAAAGGAGGGATCATG | 1189 | CTTAAATTCTGACTTGCTTG | 1391 | CTTCCATGTCTTGAATGAAG |
| 988 | CTATTTAAAGGAGGGATCAT | 1190 | GCTTAAATTCTGACTTGCTT | 1392 | GCTTCCATGTCTTGAATGAA |
| 989 | TCTATTTAAAGGAGGGATCA | 1191 | CAAGCACCATGTCTTCACTG | 1393 | TGCTTCCATGTCTTGAATGA |
| 990 | GAATGTCTCTGTAAGTTCAT | 1192 | GCAAGCACCATGTCTTCACT | 1394 | TTGCTTCCATGTCTTGAATG |
| 991 | TAGAATGTCTCTGTAAGTTC | 1193 | CAGCAACTCAGTAATACCAA | 1395 | GCAGCACTAATTAACCCTTT |
| 992 | GTACACATAGAATGTCTCTG | 1194 | TCAGCAACTCAGTAATACCA | 1396 | GAGCAGCACTAATTAACCCT |
| 993 | GGTACACATAGAATGTCTCT | 1195 | TTCAGCAACTCAGTAATACC | 1397 | TGAGCAGCACTAATTAACCC |
| 994 | TGGTACACATAGAATGTCTC | 1196 | GTTCAGCAACTCAGTAATAC | 1398 | GCAGCACTAATTAACC |
| 995 | TTGGTACACATAGAATGTCT | 1197 | GGTTCAGCAACTCAGTAATA | 1399 | GACCAAAGTAAGCCACATGG |
| 996 | GTTGGTACACATAGAATGTC | 1198 | TGGTTCAGCAACTCAGTAAT | 1400 | TGACCAAAGTAAGCCACATG |
| 997 | GGTTGGTACACATAGAATGT | 1199 | TTGGTTCAGCAACTCAGTAA | 1401 | TTGACCAAAGTAAGCCACAT |
| 998 | GGGTTGGTACACATAGAATG | 1200 | CTGTAGGAGGTAATGAGCTG | 1402 | TATTGACCAAAGTAAGCCAC |
| 999 | TGGGTTGGTACACATAGAAT | 1201 | CACTGTAGGAGGTAATGAGC | 1403 | CTATTGACCAAAGTAAGCCA |
| 1000 | TTGGGTTGGTACACATAGAA | 1202 | GGTGGTTTAGGGAATGCTGA | 1404 | TCTATTGACCAAAGTAAGCC |
| 1001 | GGTTGGTACACATAGA | 1203 | TGGTGGTTTAGGGAATGCTG | 1405 | TTCTATTGACCAAAGTAAGC |
| 1002 | GTTGGGTTGGTACACATAGA | 1204 | GTGGTGGTTTAGGGAATGCT | | |
| 1003 | GGGTTGGTACACATAG | 1205 | TAGTGGTGGTTTAGGGAATG | | |

TABLE 9
Additional sequences of ASO targeting PUFIN2.

| SEQ | | SEQ | | SEQ | |
|---|---|---|---|---|---|
| ID | | ID | | ID | |
| NO. | Sequence | NO. | Sequence | NO. | Sequence |
| 1969 | CTAGGACAATCATGGG | 1983 | GACTGTTGATTATCAC | 1997 | TTAAGGGTGCTGGTCT |
| 1970 | GCATACTCAATAGCTC | 1984 | GACATGGACTTACCTC | 1998 | CCTATAGCACTCCATA |
| 1971 | TGGCATACTCAATAGC | 1985 | TGGGTGGTTAGGCTTC | 1999 | GTTGGTCCCTATCAAA |
| 1972 | TGACTTAATAGGAGGC | 1986 | GTGGGTGGTTAGGCTT | 2000 | TAGTTGGTCCCTATCA |
| 1973 | GTTAGGCTCTTCAACC | 1987 | CCAGATTAGTGATGCC | 2001 | GTCTAGTTGGTCCCTA |
| 1974 | GGTCCTATCTCTAAGC | 1988 | CCCAGATTAGTGATGC | 2002 | GGAACTAGCCATCAGG |
| 1975 | GAGTGTCTCCAAGGGT | 1989 | GACCCAGATTAGTGAT | 2003 | GCTCAATTCCCATAAC |
| 1976 | CAAACCTTTGTAGTAC | 1990 | GGACCCAGATTAGTGA | 2004 | GTGATACTACCTTGGA |
| 1977 | GTCAAACCTTTGTAGT | 1991 | CCTAGATGCACTCCAA | 2005 | GCTTTGTGATACTACC |
| 1978 | CAATTAGCAGTAAGCC | 1992 | TCCTAGATGCACTCCA | 2006 | GTCCTAGATTACATCC |
| 1979 | GTGTGCTCTACAGGAC | 1993 | GCCAAAGTGTACCCTT | 2007 | GCAAGTCCTAGATTAC |
| 1980 | GAGCTATCTACTTAAC | 1994 | TTGCCAAAGTGTACCC | 2008 | GGTCCCTTAAATCATA |
| 1981 | TAGTATTGGATTTGCC | 1995 | CATTAGAGTTAGGGTG | 2009 | GTAAGGATATTGCCCT |
| 1982 | TAGCAGTTGGTGATCT | 1996 | CACATTAGAGTTAGGG | | |

TABLE 10
Additional sequences of ASO candidates targeting PUFIN6.

| SEQ | | SEQ | | SEQ | |
|---|---|---|---|---|---|
| ID | | ID | | ID | |
| NO. | Sequence | NO. | Sequence | NO. | Sequence |
| 41 | CAGTCCCACCAATCACCTGC | 216 | CAGTCACCACCATTCTGCTA | 391 | GTGAAATGCCCAGACATGCT |
| 42 | GTGTGCCTCTTAGACC | 217 | GATCAGTCACCACCATTCTG | 392 | GAGTGAAATGCCCAGACATG |
| 43 | TTTAAATTCCTGAGAACTGG | 218 | GGATCAGTCACCACCATTCT | 393 | GGATCATAGACTCACCCATG |
| 44 | GTTTAAATTCCTGAGAACTG | 219 | CATCTCTCCACCACAGCAGC | 394 | CAGGATCATAGACTCACCCA |
| 45 | CTGAGGTTGGAGTTGGAAGC | 220 | CCATCTCTCCACCACAGCAG | 395 | GATCATAGACTCACCC |
| 46 | GAGGTTAGGGTGTCTC | 221 | GGCTATGGACATTTGAGGGC | 396 | GGATCATAGACTCACC |
| 47 | GCTGGAGGTTAGGGTGTCTC | 222 | TCATCTTCAAGAGCCTGGAA | 397 | GACAGGATCATAGACTCACC |
| 48 | TGCTGGAGGTTAGGGTGTCT | 223 | TTCATCTTCAAGAGCCTGGA | 398 | CAGAGACAGGATCATAGACT |
| 49 | GTGCTGGAGGTTAGGGTGTC | 224 | CTACCAGAGAGCAGCAGTGA | 399 | TGTGCAGAGACAGGATCATA |
| 50 | CTAACTTCTGGACTATTTGC | 225 | CACTGCCTCCAGACATCAGG | 400 | TGTGAGTGCCTGAGTCTATC |
| 51 | GCTAACTTCTGGACTATTTG | 226 | TACACTGCCTCCAGACATCA | 401 | GTGTGAGTGCCTGAGTCTAT |
| 52 | GCTAACTTCTGGACTA | 227 | CTACACTGCCTCCAGACATC | 402 | TTTGAGTGGGTGTGAGTGCC |
| 53 | CAGCCCAAGCCTCTTAAAGA | 228 | GAACTACACTGCCTCCAGAC | 403 | GTTTGAGTGGGTGTGAGTGC |
| 54 | GCAGCCCAAGCCTCTTAAAG | 229 | GAAGAACTACACTGCCTCCA | 404 | TGGGTTTGAGTGGGTGTGAG |
| 55 | GCTAGAGTGTTGCTGG | 230 | TAAGAAGAACTACACTGCCT | 405 | GTGGGTTTGAGTGGGTGTGA |
| 56 | TCATAAGGAAAGATTGCCCT | 231 | TTAAGAAGAACTACACTGCC | 406 | GATGTGTGTGTAAGTGTGCA |
| 57 | TTCATAAGGAAAGATTGCCC | 232 | TTTAAGAAGAACTACACTGC | 407 | TAGATGTGTGTGTAAGTGTG |
| 58 | GATTCATAAGGAAAGATTGC | 233 | CCAGAAAGGTGTAAATATTC | 408 | GTAGATGTGTGTGTAAGTGT |
| 59 | CCAAGATGTAGAAGCAATCA | 234 | CCCAGAAAGGTGTAAATATT | 409 | TGTAGATGTGTGTGTAAGTG |
| 60 | CCCAAGATGTAGAAGCAATC | 235 | GCATTTATCTCCCAAACACA | 410 | GTGTAGATGTGTGTGTAAGT |
| 61 | GCCCAAGATGTAGAAGCAAT | 236 | GGCATTTATCTCCCAAACAC | 411 | GAAATGGTGGGCTGTC |
| 62 | TTGAAGCCCAAGATGTAGAA | 237 | TGGCATTTATCTCCCAAACA | 412 | CAAATCTCACTGCCCTGCCA |
| 63 | TTTGAAGCCCAAGATGTAGA | 238 | GGTCATGTTATCCATACAGG | 413 | GTTCAAATCTCACTGCCCTG |
| 64 | TTGGTGCAAGAGAGAATCCC | 239 | CAAGGTCATGTTATCCATAC | 414 | GGTCTGATACTCTGAAGTTC |
| 65 | GTTGGTGCAAGAGAGAATCC | 240 | GCAAGGTCATGTTATCCATA | 415 | GTAGGAAAGAACCAGCCTTT |
| 66 | GTGCAAGGATACTCTATATC | 241 | CAAATGAATGTAGCTCCTTA | 416 | CATAATGTTCAGGAAAGACA |
| 67 | TTTGGGAGTGCAAGGATACT | 242 | GCAAATGAATGTAGCTCCTT | 417 | TCATAATGTTCAGGAAAGAC |
| 68 | CCATTCCAACTCCCAAATTG | 243 | CAGCAAATGAATGTAGCTCC | 418 | GTTCATAATGTTCAGGAAAG |
| 69 | CAAATATTCCTAAGCTCCAT | 244 | TCAGCAAATGAATGTAGCTC | 419 | CCTAGAAGGGTGCAAC |
| 70 | CCAAATATTCCTAAGCTCCA | 245 | TATCAGCAAATGAATGTAGC | 420 | TGTTATTCTGAAGTTGGGCC |
| 71 | CCCAAATATTCCTAAGCTCC | 246 | GACTATTGTTGCATGA | 421 | TTGTTATTCTGAAGTTGGGC |
| 72 | GAAGAATTGACATCCTCAAA | 247 | TTGAATGCCCAGTACCCAGC | 422 | GTTGTTATTCTGAAGTTGGG |
| 73 | CAGAAGAATTGACATCCTCA | 248 | CATTGAATGCCCAGTACCCA | 423 | TGTTGTTATTCTGAAGTTGG |
| 74 | GCAGAAGAATTGACATCCTC | 249 | CATCAGGCAGACACAGGGTC | 424 | CCTGGCACAATCATTTGTTG |
| 75 | CACAGCAGAAGAATTGACAT | 250 | CCATCAGGCAGACACAGGGT | 425 | CCTTTGAAATTGGAGACAAT |
| 76 | CATTTGATTCTGTTCTGGAG | 251 | TCCATCAGGCAGACACAGGG | 426 | CAACTGACTTGACTTCTCTG |
| 77 | GCATTTGATTCTGTTCTGGA | 252 | TCTTTACCAGACACAAACTC | 427 | GCAACTGACTTGACTTCTCT |
| 78 | GGCATTTGATTCTGTTCTGG | 253 | GCTGTCTGTGTCTTTACCAG | 428 | GGCAACTGACTTGACTTCTC |
| 79 | GAAGGCATTTGATTCTGTTC | 254 | GATTTCATTGCTGTCTGTGT | 429 | TAGCCACTTCTTTAGATGAA |
| 80 | TGGAGGAAGGCATTTGATTC | 255 | TGATTTCATTGCTGTCTGTG | 430 | CAAATAGCCACTTCTTTAGA |
| 81 | TTGGAGGAAGGCATTTGATT | 256 | CTTCTTAGTAACCTTAATGG | 431 | GCAAATAGCCACTTCTTTAG |
| 82 | TTTGGAGGAAGGCATTTGAT | 257 | CATTCAAAGCTCTGCATTCT | 432 | TGCAAATAGCCACTTCTTTA |
| 83 | CATCTCTCAATCATGCCTAG | 258 | CCATTCAAAGCTCTGCATTC | 433 | TATGCAAATAGCCACTTCTT |
| 84 | GCCACCTAGTCATGTT | 259 | CCCATTCAAAGCTCTGCATT | 434 | TTATGCAAATAGCCACTTCT |
| 85 | TCACCCAACCACCAAAGCTT | 260 | GCCCATTCAAAGCTCTGCAT | 435 | TTTATGCAAATAGCCACTTC |
| 86 | GCTGCTCCATCACCCAACCA | 261 | TGCCCATTCAAAGCTCTGCA | 436 | GTTTATGCAAATAGCCACTT |
| 87 | CACCTCTGTGTCCCTAGAAT | 262 | CTGCCCATTCAAAGCTCTGC | 437 | TGTTTATGCAAATAGCCACT |
| 88 | CCACCTCTGTGTCCCTAGAA | 263 | TCTGCCCATTCAAAGCTCTG | 438 | GAAGCCTGTTTATGCAAATA |
| 89 | CTCTAAGTCACCAATGATCA | 264 | GTCTGCCCATTCAAAGCTCT | 439 | GGAAGCCTGTTTATGCAAAT |
| 90 | TCTCTAAGTCACCAATGATC | 265 | TGTCTGCCCATTCAAAGCTC | 440 | TGGAAGCCTGTTTATGCAAA |
| 91 | TTCTCTAAGTCACCAATGAT | 266 | CATGTCTGCCCATTCAAAGC | 441 | GATGGAAGCCTGTTTATGCA |
| 92 | CTTCTCTAAGTCACCAATGA | 267 | CCATGTCTGCCCATTCAAAG | 442 | GATCTTTCCAACTACTGGTG |
| 93 | TCTTCTCTAAGTCACCAATG | 268 | GCCATGTCTGCCCATTCAAA | 443 | TGATCTTTCCAACTACTGGT |
| 94 | TTGACTGTGCTGGGAAGGAG | 269 | TGCCATGTCTGCCCATTCAA | 444 | CTGATCTTTCCAACTACTGG |
| 95 | TTTGACTGTGCTGGGAAGGA | 270 | GGCAAACTACACCTGTGATG | 445 | CACAATAGTTCTCCAGCAAA |
| 96 | GTTTGACTGTGCTGGGAAGG | 271 | GAGGCAAACTACACCTGTGA | 446 | TCACAATAGTTCTCCAGCAA |
| 97 | GGTTTGACTGTGCTGGGAAG | 272 | GGCAAACTACACCTGT | 447 | CTCACAATAGTTCTCCAGCA |
| 98 | TGGTTTGACTGTGCTGGGAA | 273 | GGAGGCAAACTACACC | 448 | GCTCACAATAGTTCTCCAGC |
| 99 | TCTCTGGTTTGACTGTGCTG | 274 | CAGACCTGATTGTGACCCTC | 449 | CAGCTCACAATAGTTCTCCA |
| 100 | GGTTCTCTCTGGTTTGACTG | 275 | GCAGACCTGATTGTGACCCT | 450 | TCAGCTCACAATAGTTCTCC |
| 101 | TATGCAGCTCCCAGAAAGAC | 276 | GACCTGATTGTGACCC | 451 | TCTGACTCTACAGCTATAAA |
| 102 | TTATGCAGCTCCCAGAAAGA | 277 | TGCAGACCTGATTGTGACCC | 452 | TTCTGACTCTACAGCTATAA |
| 103 | TTTATGCAGCTCCCAGAAAG | 278 | CAAATGCAGACCTGATTGTG | 453 | CTTCTGACTCTACAGCTATA |
| 104 | CTTTATGCAGCTCCCAGAAA | 279 | TACAAATGCAGACCTGATTG | 454 | TCAATGAGAGAAGCCTGATC |
| 105 | GTTTCACCCAGACACCCAAA | 280 | TTACAAATGCAGACCTGATT | 455 | TGGATCAATGAGAGAAGCCT |
| 106 | GCATAGAGCAATGACC | 281 | TTTACAAATGCAGACCTGAT | 456 | CATGTAGATTTGGAAATAGG |
| 107 | CTAAGGCATAGAGCAATGAC | 282 | CAAGTATAGCAGCCTCTCAG | 457 | CTAGAAGCCATGTAGATTTG |
| 108 | CCTAAGGCATAGAGCAATGA | 283 | GTATAGCAGCCTCTCA | 458 | TGTATGTGGCTGTGTGTGTC |
| 109 | TCCTAAGGCATAGAGCAATG | 284 | GCAAGTATAGCAGCCTCTCA | 459 | TTGTATGTGGCTGTGTGTGT |
| 110 | TTCCTAAGGCATAGAGCAAT | 285 | CAAGTATAGCAGCCTC | 460 | GTTGTATGTGGCTGTGTGTG |
| 111 | TCCTAAGGCATAGAGC | 286 | CAAACTCAGCATATTGTCCC | 461 | TGTTGTATGTGGCTGTGTGT |
| 112 | GATTCTCTCTGGTTCCTAAG | 287 | CCAAACTCAGCATATTGTCC | 462 | CTGTTGTATGTGGCTGTGTG |
| 113 | CCCAGACCCTTTCAGAGTGT | 288 | CACCAAACTCAGCATATTGT | 463 | TCTGTTGTATGTGGCTGTGT |
| 114 | TTAGATATAGCCTGCT | 289 | GCACCAAACTCAGCATATTG | 464 | GTCTGTTGTATGTGGCTGTG |
| 115 | TTCCTTAGATATAGCCTGCT | 290 | GGCACCAAACTCAGCATATT | 465 | GCTGTAATTCTTTCCAACAT |
| 116 | CTTAGATATAGCCTGC | 291 | CAGGCACCAAACTCAGCATA | 466 | GAGCTGTAATTCTTTCCAAC |
| 117 | CTTCCTTAGATATAGCCTGC | 292 | CAACAGGCACCAAACTCAGC | 467 | CAAGAGCTGTAATTCTTTCC |
| 118 | CCTTAGATATAGCCTG | 293 | GCAACAGGCACCAAACTCAG | 468 | CCAAGAGCTGTAATTCTTTC |
| 119 | TCTTCCTTAGATATAGCCTG | 294 | GGCAACAGGCACCAAACTCA | 469 | GCCAAGAGCTGTAATTCTTT |
| 120 | TAGAGCCATCTTCCTTAGAT | 295 | GATCAACTACAGGGCC | 470 | CAACAGAATGAGCATAAGCC |
| 121 | GAGGCAATGGAATCTTGGAT | 296 | TGAGCAAGACATAGACCAGC | 471 | TCACAACAGAATGAGCATAA |
| 122 | CTCTCTGGAAACTCTATATT | 297 | CTGAGCAAGACATAGACCAG | 472 | TTTACCACTTCCCAGCCAGG |
| 123 | CCTCTCTGGAAACTCTATAT | 298 | TACTGAGCAAGACATAGACC | 473 | GATCTATTCACAGCTTTACC |
| 124 | TAATTGATGAAGGTCC | 299 | TTACTGAGCAAGACATAGAC | 474 | CATCTAGCTCATGCCTACAC |
| 125 | TGCCATCTCAGCACCTCAGC | 300 | TTTACTGAGCAAGACATAGA | 475 | CAGCTCTGCAACTTCCTAGC |
| 126 | CTGCCATCTCAGCACCTCAG | 301 | CATTTACTGAGCAAGACATA | 476 | TCAGCTCTGCAACTTCCTAG |
| 127 | TTCAAGAACTGCCATCTCAG | 302 | CCATTTACTGAGCAAGACAT | 477 | TCAATATTAGAGAATGGGCC |
| 128 | GACCACCACTCTAAATGTCT | 303 | TCCATTTACTGAGCAAGACA | 478 | TGATGACTGACTGAATGAGT |
| 129 | TAGACCACCACTCTAAATGT | 304 | TTCCATTTACTGAGCAAGAC | 479 | TTGATGACTGACTGAATGAG |
| 130 | CTAGACCACCACTCTAAATG | 305 | CTTCCATTTACTGAGCAAGA | 480 | GTTGATGACTGACTGAATGA |
| 131 | CCTAGACCACCACTCTAAAT | 306 | CATCATCCTTTGTGTCCAGA | 481 | CACAGCAGTCAGTAATAGTT |
| 132 | CCCTAGACCACCACTCTAAA | 307 | CATTTGAACCCTGTCACCCA | 482 | CAACACAGCAGTCAGTAATA |
| 133 | GCCCTAGACCACCACTCTAA | 308 | GCATTTGAACCCTGTCACCC | 483 | TGGGCACAACACAGCAGTCA |
| 134 | CTAGACCACCACTCTA | 309 | TGCATTTGAACCCTGTCACC | 484 | TTGGGCACAACACAGCAGTC |
| 135 | TGCCCTAGACCACCACTCTA | 310 | CTGCATTTGAACCCTGTCAC | 485 | CAGAGCCAATATCCACATGT |
| 136 | CTCATACTACCAAGCCCTGC | 311 | CACTGCATTTGAACCCTGTC | 486 | GAGACACACAGAGCCATATA |
| 137 | CCTCATACTACCAAGCCCTG | 312 | TAAACACTGCATTTGAACCC | 487 | GGAGACACACAGAGCCATAT |
| 138 | GTCACTAACCCTCATACTAC | 313 | TTAAACACTGCATTTGAACC | 488 | CAGGAGACACACAGAGCCAT |
| 139 | GGTCACTAACCCTCATACTA | 314 | CTTAAACACTGCATTTGAAC | 489 | CAAGCTCTGATCTGTTGTCC |
| 140 | GGTCACTAACCCTCAT | 315 | CACTTAAACACTGCATTTGA | 490 | CCAAGCTCTGATCTGTTGTC |
| 141 | CATGATGTAGCAACCC | 316 | CCACTTAAACACTGCATTTG | 491 | TGTCCATATTGGTGAC |
| 142 | CCATGATGTAGCAACC | 317 | CCCACTTAAACACTGCATTT | 492 | TAGATGTCCATATTGGTGAC |
| 143 | CCCATGATGTAGCAAC | 318 | TCCCACTTAAACACTGCATT | 493 | CTAGATGTCCATATTGGTGA |
| 144 | GCCTGGAACTGGACATTGGG | 319 | CTCCCACTTAAACACTGCAT | 494 | GATGTCCATATTGGTG |
| 145 | GCTTGGAAACTGTCTGCCTC | 320 | CCTCCCACTTAAACACTGCA | 495 | CCAGTGTAAAGGGTGC |
| 146 | TGCTTGGAAACTGTCTGCCT | 321 | CAATCTATGTCAAGCTTCCC | 496 | CTGTGGTCCTTTGTGAAGAG |
| 147 | GATGCTTGGAAACTGTCTGC | 322 | TCAATCTATGTCAAGCTTCC | 497 | CCTGTGGTCCTTTGTGAAGA |
| 148 | GGATGCTTGGAAACTGTCTG | 323 | CTCAATCTATGTCAAGCTTC | 498 | TTCAGGGACAAGGATAGCCT |
| 149 | TAAAGACACATCTTCAGCCT | 324 | CACTCAATCTATGTCAAGCT | 499 | TAGTATTCAGGGACAAGGAT |
| 150 | TTAAAGACACATCTTCAGCC | 325 | TCACTCAATCTATGTCAAGC | 500 | TTAGTATTCAGGGACAAGGA |
| 151 | GCAGAAATTAAAGACACATC | 326 | CTCACTCAATCTATGTCAAG | 501 | CATTAGTATTCAGGGACAAG |
| 152 | TTTCCTAAAGATGAGGTGGC | 327 | TCTCACTCAATCTATGTCAA | 502 | GACATTAGTATTCAGGGACA |
| 153 | GTTTCCTAAAGATGAGGTGG | 328 | GCCACCACTGAGTATAACTC | 503 | TAGACATTAGTATTCAGGGA |
| 154 | GAGTTTCCTAAAGATGAGGT | 329 | CAGTCACCAACTCTCTATGG | 504 | TTAGACATTAGTATTCAGGG |
| 155 | CAGAGTTTCCTAAAGATGAG | 330 | GCAGTCACCAACTCTCTATG | 505 | GACCTTTAGACATTAG |
| 156 | TCAGAGTTTCCTAAAGATGA | 331 | CAAAGGCAGTCACCAACTCT | 506 | GGCTGACCTTTAGACATTAG |
| 157 | TTCAGAGTTTCCTAAAGATG | 332 | TCAAAGGCAGTCACCAACTC | 507 | GGCTGACCTTTAGACA |
| 158 | CTTCAGAGTTTCCTAAAGAT | 333 | CTCAAAGGCAGTCACCAACT | 508 | CACATCACATGCACACTGCT |
| 159 | GCTTCAGAGTTTCCTAAAGA | 334 | TCTCAAAGGCAGTCACCAAC | 509 | GACACATCACATGCACACTG |
| 160 | TTCCACAAGAAGCCAGAGCT | 335 | CATTCTCCTCTCAAAGGCAG | 510 | GGACACATCACATGCACACT |
| 161 | CATTCCACAAGAAGCCAGAG | 336 | CCATTCTCCTCTCAAAGGCA | 511 | GGGACACATCACATGCACAC |
| 162 | TACATTCCACAAGAAGCCAG | 337 | GGATCACAACCATTCTCCTC | 512 | TGGGACACATCACATGCACA |
| 163 | TTACATTCCACAAGAAGCCA | 338 | GGTATAAGTCCTCTGGGTGA | 513 | GACACAGCTTCTTCCTCCAA |
| 164 | CTTACATTCCACAAGAAGCC | 339 | TATAAGTCCTCTGGGT | 514 | TGATGGGAGGGACACAGCTT |
| 165 | TCTTACATTCCACAAGAAGC | 340 | GTATAAGTCCTCTGGG | 515 | GTTATACCCTCAAAGAACCC |
| 166 | GTCTTACATTCCACAAGAAG | 341 | GGTATAAGTCCTCTGG | 516 | GGCATCATACCTTCAC |
| 167 | CAAAGTCTTACATTCCACAA | 342 | TCCTTCTGCTGTCACCAAGG | 517 | GGGAGGGCATCATACC |
| 168 | GACAAAGTCTTACATTCCAC | 343 | GTCCTTCTGCTGTCACCAAG | 518 | GGAAGTAAGAACCCATCTGG |
| 169 | GCTGATGACTTTACCATTTC | 344 | GAGTCCTTCTGCTGTCACCA | 519 | TTTGTGGAATGCTGGATATT |
| 170 | TGCTGATGACTTTACCATTT | 345 | TATCCATGAATTCCTCCCAA | 520 | CTTTGTGGAATGCTGGATAT |
| 171 | CTGCTGATGACTTTACCATT | 346 | CATCTCCATATCCATGAATT | 521 | TCTTTGTGGAATGCTGGATA |
| 172 | TAACTGCTGATGACTTTACC | 347 | TCATCTCCATATCCATGAAT | 522 | TTCTTTGTGGAATGCTGGAT |
| 173 | CTAACTGCTGATGACTTTAC | 348 | TTCATCTCCATATCCATGAA | 523 | TGATTGGGCAATATCC |
| 174 | CCTAACTGCTGATGACTTTA | 349 | GTTCATCTCCATATCCATGA | 524 | GTGATTGGGCAATATC |
| 175 | CCCTAACTGCTGATGACTTT | 350 | GTGTTATCTGGTCTAC | 525 | GGTGATTGGGCAATAT |
| 176 | TCCCTAACTGCTGATGACTT | 351 | GATATTTCCACCTAGAGACT | 526 | TGGTGATTGGGCAATA |
| 177 | TTCCCTAACTGCTGATGACT | 352 | TGATATTTCCACCTAGAGAC | 527 | CAGATGTCCATGTTGGTGAC |
| 178 | TTTCCCTAACTGCTGATGAC | 353 | GTGATATTTCCACCTAGAGA | 528 | TCAGATGTCCATGTTGGTGA |
| 179 | GAGCAACAGAACCTCAACTG | 354 | TGTGATATTTCCACCTAGAG | 529 | TTCAGATGTCCATGTTGGTG |
| 180 | TCTGGAGAATTGGAGTTGGG | 355 | CTGTGATATTTCCACCTAGA | 530 | GTTCAGATGTCCATGTTGGT |
| 181 | TATCATCAGTCTAGGG | 356 | TCTGTGATATTTCCACCTAG | 531 | CTCCAAATACATCCAGAGAT |
| 182 | TCTTTATCATCAGTCTAGGG | 357 | TAGCAGAATGTCTCTTATTC | 532 | GCTCCAAATACATCCAGAGA |
| 183 | CTCTTTATCATCAGTCTAGG | 358 | CTAGCAGAATGTCTCTTATT | 533 | AGCTCCAAATACATCCAGAG |
| 184 | TGCTCCAGGCTCCCTCAACA | 359 | TCTAGCAGAATGTCTCTTAT | 534 | GGGATATATGGGAACTTTCT |
| 185 | CAAACACTCAGCATGGACAA | 360 | TGATCCCTCAATCCTAGCTG | 535 | AGGGATATATGGGAACTTTC |
| 186 | TCAAACACTCAGCATGGACA | 361 | GATCCCTCAATCCTAG | 536 | AAGGGATATATGGGAACTTT |
| 187 | TTCAAACACTCAGCATGGAC | 362 | TCATAGGGTTCTTACC | 537 | TAAGGGATATATGGGAACTT |
| 188 | CTTCAAACACTCAGCATGGA | 363 | GTCATAGGGTTCTTAC | 538 | TGATGATGTAGGAGGTTAAC |
| 189 | GCTTCAAACACTCAGCATGG | 364 | GAAGTGTCATAGGGTTCTTA | 539 | CATGATGATGTAGGAGGTTA |
| 190 | GGCTTCAAACACTCAGCATG | 365 | GTGTCATAGGGTTCTT | 540 | GCAAATGTTCCATGATGATG |
| 191 | CAGGCTTCAAACACTCAGCA | 366 | TGAAGTGTCATAGGGTTCTT | 541 | GGCAAATGTTCCATGATGAT |
| 192 | CACAGGCTTCAAACACTCAG | 367 | TTGAAGTGTCATAGGGTTCT | 542 | TGGCAAATGTTCCATGATGA |
| 193 | TCACAGGCTTCAAACACTCA | 368 | CTTGAAGTGTCATAGGGTTC | 543 | TTGGCAAATGTTCCATGATG |
| 194 | CTCACAGGCTTCAAACACTC | 369 | GAAGTGTCATAGGGTT | 544 | TTCACAGCAAAGCACCAGTG |
| 195 | TCTCACAGGCTTCAAACACT | 370 | TCTTGAAGTGTCATAGGGTT | 545 | GTTCACAGCAAAGCACCAGT |
| 196 | GAACTCCTGACCCAAGTGCC | 371 | TTGAAGTGTCATAGGG | 546 | TAGTTCACAGCAAAGCACCA |
| 197 | TGAACTCCTGACCCAAGTGC | 372 | CTTGAAGTGTCATAGG | 547 | TTAGTTCACAGCAAAGCACC |
| 198 | CTGAACTCCTGACCCAAGTG | 373 | GGGATTATGTGGGATC | 548 | TTTAGTTCACAGCAAAGCAC |
| 199 | TCTGAACTCCTGACCCAAGT | 374 | CAACTGGGATTATGTGGGAT | 549 | GAGTTTAGTTCACAGCAAAG |
| 200 | CTCTGAACTCCTGACCCAAG | 375 | CAGGTTTGAGGAGGTTTGAG | 550 | GGAGTTTAGTTCACAGCAAA |
| 201 | CCTCTGAACTCCTGACCCAA | 376 | CAACCCACTTCATTGTCATG | 551 | CAAATGCAATGTGATATCCT |
| 202 | TTCAGTTACCATCATCCTGG | 377 | CTACAATGACCTCAGAGCCC | 552 | CCAAATGCAATGTGATATCC |
| 203 | CAGCTTCAGTTACCATCATC | 378 | GCTACAATGACCTCAGAGCC | 553 | CCAAGGAGACACAAACACCA |
| 204 | CCAGCTTCAGTTACCATCAT | 379 | GGCTACAATGACCTCAGAGC | 554 | TGGAATACCTGACCAGTACT |
| 205 | CCCAGCTTCAGTTACCATCA | 380 | TGGCTACAATGACCTCAGAG | 555 | TATGGAATACCTGACCAGTA |
| 206 | TGAGACAACACCATCCACAA | 381 | GAACTCTGCTTCTTGTGAAT | 556 | CTATGGAATACCTGACCAGT |
| 207 | CTGAGACAACACCATCCACA | 382 | GGAACTCTGCTTCTTGTGAA | 557 | GCTAACAGTAGGACATTCTA |
| 208 | TCTGAGACAACACCATCCAC | 383 | TGGAACTCTGCTTCTTGTGA | 558 | GCTAACAGTAGGACAT |
| 209 | GCAGCATATAACTCTCCTTC | 384 | CATGGAACTCTGCTTCTTGT | 559 | GGATCATTTCTCATCACTGC |
| 210 | CCATTCTGCTAAGTCCAGGG | 385 | CTAGATCTTACAAAGGACCC | | |
| 211 | CACCATTCTGCTAAGTCCAG | 386 | GACTAGATCTTACAAAGGAC | | |
| 212 | CCACCATTCTGCTAAGTCCA | 387 | CTTGGGAAGGTAGAATCTGG | | |
| 213 | CACCACCATTCTGCTAAGTC | 388 | GAAGGACACTTGGGCAGATT | | |
| 214 | TCACCACCATTCTGCTAAGT | 389 | TCAGAAGGACACTTGGGCAG | | |
| 215 | GTCACCACCATTCTGCTAAG | 390 | TTCAGAAGGACACTTGGGCA | | |

TABLE 11
Sequences of ASO candidates targeting PUFIN4.

| SEQ | | SEQ | | SEQ | |
|---|---|---|---|---|---|
| ID | | ID | | ID | |
| NO. | Sequence | NO. | Sequence | NO. | Sequence |
| 2010 | AGTGCTACCCACAAGG | 2012 | TTGATGCTAACAGACC | 2013 | GGACTACACCTTGACA |
| 2011 | GGACATGCTATTGATG | | | | |

TABLE 12
Sequences of ASO candidates targeting PUFIN8.

| SEQ | | SEQ | | SEQ | |
|---|---|---|---|---|---|
| ID | | ID | | ID | |
| NO. | Sequence | NO. | Sequence | NO. | Sequence |
| 2014 | TTAGACCTAGACTGCT | 2016 | CCATTAGAGTAGTAGG | 2018 | GAAACCTTAGTGTGTC |
| 2015 | GCCTTAGATCAGTCCT | 2017 | GTTACCAGACCTCTAC | | |

TABLE 13
Sequences of ASOs targeting PUFIN9.

| SEQ | | SEQ | | SEQ | |
|---|---|---|---|---|---|
| ID | | ID | | ID | |
| NO. | Sequence | NO. | Sequence | NO. | Sequence |
| 2019 | GTCCTAACAGAGTAAC | 2038 | AGCCTAGCTTTAACTA | 2057 | ATTGTTGTTAGAACCC |
| 2020 | TTGAGCTACTAATGCC | 2039 | ACACCCTTAATGAGAC | 2058 | GGAGTTGCATAGCATG |
| 2021 | GTTGAGCTACTAATGC | 2040 | GACACCCTTAATGAGA | 2059 | TGGAGTTGCATAGCAT |
| 2022 | TGTTGAGCTACTAATG | 2041 | GGACACCCTTAATGAG | 2060 | GAGTTACATTAACCAC |
| 2023 | TCATATCATGTTGAGC | 2042 | AGGACACCCTTAATGA | 2061 | GGGAGTTACATTAACC |
| 2024 | GTCATATCATGTTGAG | 2043 | CTAATGATGTGAGGAC | 2062 | GTAGCCCAGCTCTTTA |
| 2025 | GAACTACTTGCCTACT | 2044 | TATTATAGATCCCAGC | 2063 | TGGACCTTGAGTTGTC |
| 2026 | CCTCTTGACCACTAGG | 2045 | ATTAGGTGCTTTAGGG | 2064 | CTGGACCTTGAGTTGT |
| 2027 | GCCTCTTGACCACTAG | 2046 | GCTGGTGGTTAGATAT | 2065 | GCTAGGACCAATATTC |
| 2028 | GTAGATCAAGTGTCTT | 2047 | AGTCCCTACAAGATAG | 2066 | TACTGCTCCATTAGGG |
| 2029 | GAGTAGATCAAGTGTC | 2048 | GCAGTCCCTACAAGAT | 2067 | ATACTGCTCCATTAGG |
| 2030 | GTTAGCTGTAAACTAC | 2049 | GAGCAGTCCCTACAAG | 2068 | GTAGTCCTTATTTGAC |
| 2031 | GCATAGACTCACCTGT | 2050 | TGTAAGCTCATAAGGC | 2069 | GTCTCTGATGTAGTCC |
| 2032 | GGTGAACTTAGTACTT | 2051 | ATCAGGGTAGCTTTAG | 2070 | GTACCTTGACATCACA |
| 2033 | TGGTGAACTTAGTACT | 2052 | GTCATCAGGGTAGCTT | 2071 | AGTACCTTGACATCAC |
| 2034 | CTGGTGAACTTAGTAC | 2053 | TGTTAGAACCCTACTT | 2072 | CTTACAGTACCTTGAC |
| 2035 | GATCTGGAGGGCTTAA | 2054 | TTGTTAGAACCCTACT | 2073 | GCCACTTACAGTACCT |
| 2036 | GATATGCCTGATCCAG | 2055 | GTTGTTAGAACCCTAC | | |
| 2037 | GCCTAGCTTTAACTAT | 2056 | TGTTGTTAGAACCCTA | | |

TABLE 14
Sequences of ASOs targeting PUFIN10.

| SEQ | | SEQ | | SEQ | |
|---|---|---|---|---|---|
| ID | | ID | | ID | |
| NO. | Sequence | NO. | Sequence | NO. | Sequence |
| 2074 | GTAGCTGTGTGGGACC | 2075 | GCTATTGAGACATCCC | 2076 | AGCTATTGAGACATCC |

TABLE 15
Sequences of ASO candidates targeting PUFIN11.

| SEQ | | SEQ | | SEQ | |
|---|---|---|---|---|---|
| ID | | ID | | ID | |
| NO. | Sequence | NO. | Sequence | NO. | Sequence |
| 2077 | GGCTACCCTTCAAGAG | 2156 | GCCTGCTAACAATCTG | 2235 | ACCCTAACATTGGAGT |
| 2078 | AACCTGTCCAATACCC | 2157 | TGGCACCCTATTTCAG | 2236 | GTTGCCAATAGAGTAT |
| 2079 | TATAGCCAGTAGTTGT | 2158 | GCACCAATCCACCATT | 2237 | CCACTATACAGCTTGA |
| 2080 | CCATAGATAGCTCTAG | 2159 | GTACCTTTAGCACACT | 2238 | GGATACCACTTGATCA |
| 2081 | GACTCATCCATAGCCC | 2160 | AGACTAGACTCATCAC | 2239 | TACACACCTAGTACTC |
| 2082 | CTAACCACTCTAGTAG | 2161 | GCATAAGGTCCCATAG | 2240 | GCAACATTCATAGCCC |
| 2083 | GTGATACAATAGGTTG | 2162 | CCTGGTAAAGCACTAC | 2241 | TAGCCACCAGCTTTAG |
| 2084 | GCTAACCACTCTAGTA | 2163 | CCTGACAAGAGATAGC | 2242 | GACAGCCTACTACCAA |
| 2085 | GGCTAACCACTCTAGT | 2164 | GCCCTCCTACTAATGG | 2243 | GCCTCAACAATGCCTA |
| 2086 | GAGGCATTACTAACCC | 2165 | CTAGATTCTTGGAGCC | 2244 | GCTGATTACCCTACAT |
| 2087 | GGAGGCATTACTAACC | 2166 | GATCCTAGTTAAGCCA | 2245 | GCAGCCTTGATAGTGC |
| 2088 | CCCAGTCTAGTACAGC | 2167 | GCCTAAGATTATCCCT | 2246 | TGTTGCACCTCTTAGG |
| 2089 | CACCCTTAGTATGAGT | 2168 | GAGCCAATAATATAGC | 2247 | ACACCTAGTACTCTAC |
| 2090 | TGAATAGACTCATCCC | 2169 | AGTCCTCCTTATGATA | 2248 | GATATTGCCTATCTGC |
| 2091 | TAGTTAAGCCATGCCA | 2170 | AGTAGACCTTCCATTG | 2249 | GTATATGTTCTGGCAC |
| 2092 | GCAGTCCTACTCAGTG | 2171 | TGGAACTACTCTGGTC | 2250 | GCTAGAAGTGCCTAAC |
| 2093 | AGCCTTGTGTGTACCC | 2172 | GCTACTAACAGAAGGT | 2251 | ATGTTGCACCTCTTAG |
| 2094 | AGCTAGGGTTATACTC | 2173 | GAGCCCAACTTACACC | 2252 | GTATAACTCTTAGTCC |
| 2095 | AGCCCATGTACTAAGC | 2174 | GTCCTCCTTATGATAG | 2253 | AGCACCCTGATAGCAC |
| 2096 | AGCTCCATTAGTCCTA | 2175 | AGTACCATCAACTGAC | 2254 | CTTGAGAGTAGTCCCT |
| 2097 | TAGCCTCCTTTATAGG | 2176 | GGTATCTACAGTGCAA | 2255 | CAGGTTCCAACTAAGT |
| 2098 | AAGGTCCCATAGTGAC | 2177 | ACTTTGGGTGGTGTCC | 2256 | GTTAATCCAGCACTAG |
| 2099 | TCTGGTCAAGAGTTGC | 2178 | GATAAGCTCACTAGGT | 2257 | TAGATTGGCTTGATGC |
| 2100 | ATTAGCCCTCAGCAGT | 2179 | GGCACCAACTCTATCC | 2258 | AGCAAGTAGCCAATTG |
| 2101 | TATTAGCCCTCAGCAG | 2180 | AGCACCCTTGTTTGGC | 2259 | GCTACTAACACCATGC |
| 2102 | AGGCTACCCTTCAAGA | 2181 | AGCTTTACCAGCCCTA | 2260 | GACTCATCACCTGGAC |
| 2103 | GAGATTACTCCTGATA | 2182 | CCTCACATATAGGACC | 2261 | ACCTCACATATAGGAC |
| 2104 | TGAGACTACTACCTGC | 2183 | TGAGCTAGGGATTACC | 2262 | TCTGGGACAGTTAGCC |
| 2105 | TAAGGTCCCATAGTGA | 2184 | GCACCCTTGTTTGGCA | 2263 | GACTACCTTAGCTGTC |
| 2106 | GTATTGTTACCTAGAC | 2185 | CTGTAGGCATGGTATA | 2264 | GGTTTACACCCATGAC |
| 2107 | AGTGTAGCAACCTTAC | 2186 | TACACCATAGGCTACC | 2265 | CTAGGCAACTTAAAGC |
| 2108 | AACTAAGGACCAATCC | 2187 | GAGACTTGTGGTAGCT | 2266 | CTAAGATTATCCCTAG |
| 2109 | CATAAGGTCCCATAGT | 2188 | GGACTCTGATCTACCA | 2267 | CTCCTAGCTTACCAGA |
| 2110 | ATTAAGCCTCAACTAG | 2189 | TGAGCCCAACTTACAC | 2268 | TAGCCTAATAAGACTC |
| 2111 | TGAGCACTTAGTACCC | 2190 | CCTACTAATGGAGTTA | 2269 | GTGACTATAGACCTTC |
| 2112 | CTAGTTGGTGTCCCTA | 2191 | AGCATAAGGTCCCATA | 2270 | TACACCACTTGCATGA |
| 2113 | GATAGTCATCCAGCCA | 2192 | GATGTACCTTTAGCAC | 2271 | TATCAAGAGCCAGTAC |
| 2114 | GGTATCTGACACTCCT | 2193 | TTGAATGGACTCAACC | 2272 | AAGTGTCCTACATAGC |
| 2115 | ACCCTGATAGCACTAC | 2194 | GCCAATCCCAGATTAC | 2273 | GAAGAGTACATATCCC |
| 2116 | GCCAGCTAACCACAAC | 2195 | GGCACCATTCAATTGC | 2274 | ACTAGATTCTTGGAGC |
| 2117 | CCCTGATAGCACTACC | 2196 | GTGTAATACTATCCTG | 2275 | GTCCTAGTTTGACCAC |
| 2118 | GCTTAGATGATATCCC | 2197 | GGGACTCTGATCTACC | 2276 | TTGACCAACAATAGCC |
| 2119 | CCTAATGTAGCCACAC | 2198 | TGCTCCCAGTAGACTA | 2277 | CTGTAAGGGTGGCAAC |
| 2120 | GGCATACCAAGTTGAA | 2199 | TACTAGAGCATCCTGC | 2278 | GTCACTACCCTAAGAG |
| 2121 | GCCCAATGATGATGCA | 2200 | AGCCTTGACCCACTTA | 2279 | TAGGTATCCCTCTCAA |
| 2122 | GGAACTACTCTGGTCA | 2201 | GTTGGGTAGCAGTGAC | 2280 | GGACTCTACAGCAGGT |
| 2123 | GTACACCACTTGCATG | 2202 | AGTATACCACATTGGC | 2281 | CAATCATGGAATGTCC |
| 2124 | CATAGAACCTTGGCTC | 2203 | GCATACAACAATGGGT | 2282 | CTTGTACCTAGAGCCT |
| 2125 | CTCACATATAGGACCC | 2204 | CCCTAGCCAATCTTAG | 2283 | GATAGTCAATTCAGTC |
| 2126 | CCTGATAGCACTACCA | 2205 | ATACCCTTGTAGAGAG | 2284 | CTCCTAATGACCTAGC |
| 2127 | AGCCTGCTAACAATCT | 2206 | TAGCATAAGGTCCCAT | 2285 | TGTTAGCCCATCTAGG |
| 2128 | GGCTCCAAGTTGTTAC | 2207 | GACTTTGGTTGACACC | 2286 | GACCATGACCAGACTA |
| 2129 | GTAGCCTCCTTTATAG | 2208 | GAGGTCTACAATCACC | 2287 | GCTTAATCAGCCTTAC |
| 2130 | AGAGACTTGTGGTAGC | 2209 | ACCCTCTGGTAAGTCC | 2288 | ATAGCTGATTACCCTA |
| 2131 | ATTGCCAGTACTTGAC | 2210 | GGCTGAACCCTAACTG | 2289 | GCTCCAGTAAGTGTAC |
| 2132 | GCCACTAAGAGCAGTA | 2211 | CTACTAGAGCATCCTG | 2290 | ACCAATCCTTAGACCA |
| 2133 | GTGTAGCAACCTTACC | 2212 | GTAGCAACCTTACCTG | 2291 | GGCTCTATCTAACCTG |
| 2134 | AGCTTGGACCTATTGC | 2213 | GCCACTATACAGCTTG | 2292 | AGTGACTATAGACCTT |
| 2135 | GTCCAGAATAGCATCC | 2214 | GATGGGATGGCTCTAA | 2293 | GACTATAGACCTTCTA |
| 2136 | TGTAGCCCTGTGGACC | 2215 | GACTGAGACTACTACC | 2294 | GTTGGTGCTTTGTCAC |
| 2137 | ATCCTAGAGTTTGACC | 2216 | GTACATGACTACCTTA | 2295 | GCATCTAACCTCTTAG |
| 2138 | GACACTCCTCTGATAG | 2217 | CTAGATGTAGCAGGTG | 2296 | GACTCACTAAGTACCT |
| 2139 | TCTGAAGGACCATAGC | 2218 | CTACTAACAGAAGGTC | 2297 | GCACCAGTAGTCACCT |
| 2140 | CTATTATAGAGCCAAC | 2219 | ACTCCAGTACCATAGT | 2298 | ATCCAGTCAACCTAGC |
| 2141 | CACTCCAGTACCATAG | 2220 | GCCCATCAGATACTTG | 2299 | CATTAGTCTGCCCAAC |
| 2142 | CAATCTGGAATAGGGC | 2221 | GTGCTAACTATACCAG | 2300 | CCAATCCTTAGACCAC |
| 2143 | GTACACCAGAGATATC | 2222 | GTGCTCACCAATAAGG | 2301 | GATGACTTAGACATGC |
| 2144 | GTACAGGACCCAGACC | 2223 | CATAGCATAAGGTCCC | 2302 | CCATACACTGGTTGCA |
| 2145 | TATTATAGAGCCAACC | 2224 | TATTAGTGCAGCCCTT | 2303 | CCTCCTTATGATAGAG |
| 2146 | GTGAATAGACTCATCC | 2225 | CATAGATAGCTCTAGG | 2304 | GTGGTTCTTTAGGCTC |
| 2147 | TCCATACACTGGTTGC | 2226 | CCTAGCTTACCAGAGG | 2305 | CTAAGGGAGGTTCACT |
| 2148 | ATTATAGAGCCAACCC | 2227 | TACCCTTGTAGAGAGC | 2306 | CCCAGTTGTATGTAGG |
| 2149 | CTAACAAGTAGTGTCT | 2228 | ACCTTAGCTGTCCAAC | 2307 | GCACCCTGATAGCACT |
| 2150 | GTAGACCTTCCATTGC | 2229 | GATACACTAGCTCAGC | 2308 | AGGTTCCAACTAAGTC |
| 2151 | GTAGCTCATGGATCAC | 2230 | CCATAGCATAAGGTCC | 2309 | GAGTCACCAACCATGA |
| 2152 | CCATAGGAAATAGCCC | 2231 | GGCTTGTATCACCCAA | 2310 | ATAGGTATCCCTCTCA |
| 2153 | GCAGACTAGGCTCCTT | 2232 | TGCTCACCAATAAGGC | | |
| 2154 | GAGCACTTAGTACCCA | 2233 | TAGCAACCTTACCTGC | | |
| 2155 | ATTGTCCCTACACCAG | 2234 | ACTAGGCAATGGCACC | | |

TABLE 16
Sequences of ASOs targeting PUFIN12.

| SEQ | | SEQ | | SEQ | |
|---|---|---|---|---|---|
| ID | | ID | | ID | |
| NO. | Sequence | NO | Sequence | NO. | Sequence |
| 2311 | GTGTACTCAGACCTTG | 2396 | CAGTTGAGGGCAGTAC | 2481 | GTAGACCCAGACCCTA |
| 2312 | CTTATCCAGTGCAACC | 2397 | CTATCAAACCATAGGC | 2482 | GTAAGTTCAGATGGCC |
| 2313 | CTACACTACAGAGCCC | 2398 | GTTTGATTGCAACCCT | 2483 | TGGCAATTAGATTACC |
| 2314 | GGGCATCCTCTAGGTT | 2399 | GCTAACCAATCTTTGC | 2484 | GTACACTTGCTAAGAC |
| 2315 | GCACATAAGACCCTGC | 2400 | GGATCAACCTGCTGCC | 2485 | GCACCATTGAGCTATC |
| 2316 | GGTCCCTAACAATGCC | 2401 | GCAAGACTGTAGAGCC | 2486 | GAACCTTGTGTCCTAA |
| 2317 | GCCTGGAAGTTAAGCC | 2402 | GAGCAACTTCAGGTAC | 2487 | CTGGAGCATTAAACCT |
| 2318 | GACTATATCAACCACC | 2403 | ACTTGTATGGCACCTA | 2488 | CTTGACCATTAGCTGC |
| 2319 | GGTCCCAACTCTAACC | 2404 | CCAAGTAATGGTGTGC | 2489 | GATGCCAGACTTGTAT |
| 2320 | GGATCAACCAAACCCT | 2405 | ATGCCTGACTAAGTGG | 2490 | ACTGATGATAGTGCTC |
| 2321 | GACACTAGACTGCATC | 2406 | GCATAATGACCTGACC | 2491 | GGACTAACCAACCACC |
| 2322 | CATAATGACCTGACCC | 2407 | GCAGGTATGACTACTC | 2492 | GATCCAACTGAACCAC |
| 2323 | GACCACTTACTAATGG | 2408 | AGGTTGTCAAGGGTCC | 2493 | TCTGGTTAGGTATGAC |
| 2324 | GTCCATGAGTACCAGC | 2409 | ATGCCCTTGGTTATAG | 2494 | AGCCTACAGTTAGATC |
| 2325 | GCATTCTATACAGCCC | 2410 | TGCTAGACTCAGGTGT | 2495 | GCCCTGCCACTAGATA |
| 2326 | GCACATAAGACCCTGC | 2411 | TTGTCCTAGCCAAGAC | 2496 | GCCTAGTTAAACACTC |
| 2327 | GATGCCAGTCTAACCT | 2412 | GTTGCCACTTGAGACT | 2497 | AATAGTGCTCTCCTAC |
| 2328 | CTAGCCTCACACTAAC | 2413 | TCTAGGGTAGGCTGAC | 2498 | GTACCAGGGCAATCAT |
| 2329 | GAGTTTAGGGCATACC | 2414 | GCACCATAAGAATCTC | 2499 | GAGACTTGCAGCCTAC |
| 2330 | GTCTAGCCTCACACTA | 2415 | ACTGGTGTTAGAGAGC | 2500 | GGAGTTTATCACAACC |
| 2331 | GAGTATCACTACCCTC | 2416 | ATGCCAGTCTAACCTG | 2501 | GAACTCTAGTCCACTT |
| 2332 | TGCCCTTGGTTATAGG | 2417 | CTAGTCAAGAACCAAC | 2502 | ACTCTAGTCCACTTGG |
| 2333 | CAGCCCATTAGTCATG | 2418 | GAGCATATTACTCCTC | 2503 | AACTCTAGTCCACTTG |
| 2334 | GATACCAAGTAGCATC | 2419 | GTCCAGAGCATATTAC | 2504 | GGCTAATCCTTTGCAC |
| 2335 | TGTAACCCATACAACC | 2420 | GACCCACAATGCTGAC | 2505 | TACCAGGGCAATCATC |
| 2336 | GGGCAAACATCTAGTG | 2421 | GGACTATATCAACCAC | 2506 | CCTTATCCAGTGCAAC |
| 2337 | GGATCACCCAACCAGG | 2422 | GCATACTCAGATTATC | 2507 | GTATGCCAGTTCACAC |
| 2338 | GTTGTTGACCCATTAC | 2423 | CACTAGTCCAGTCAAG | 2508 | AGCTACTGACCATGTC |
| 2339 | TGTTGACCCATTACTG | 2424 | GCCCTATCCACAAGGT | 2509 | AGTCCCACATTGTAGC |
| 2340 | CCAGCATGATAGGGTC | 2425 | GAATACACCAGCTTGC | 2510 | TGTTGTGTGACCACCC |
| 2341 | GTAACCCATACAACCC | 2426 | GAGTCATTGTTTGCCC | 2511 | TCATTGGTAGGTCAGA |
| 2342 | GTTGACCCATTACTGG | 2427 | GCATTGAGACACCCAC | 2512 | GACTTGATAAGCCCAC |
| 2343 | GTCTAGCCTCATACTG | 2428 | CCTACTAAGCCAAGCT | 2513 | GATCCTGATTGAGACC |
| 2344 | GTAAGTCCCATGACCC | 2429 | GGTCTGGTCAATGTCC | 2514 | AATGGGTGTAGGTTCC |
| 2345 | CCCATAGAGGATGCCC | 2430 | TGTTTGATTGCAACCC | 2515 | CTGATGATAGTGCTCC |
| 2346 | TTGTACCTACTAAGCC | 2431 | GACCACTCATCAGCCC | 2516 | AGACCATCATACCACC |
| 2347 | CCATCAAGCTCCTAGC | 2432 | GTAGTGTGGAGTCATG | 2517 | CAAAGTGCCCTACTGC |
| 2348 | GAGTTTATCACAACCC | 2433 | TGGACTCTATGCACCC | 2518 | TGAGAGTGGACCTAAT |
| 2349 | CTCTCATAGGATGCCC | 2434 | GGATGCTTGACTCTCC | 2519 | CTAACCCATACTGAGC |
| 2350 | GCACATAAGACCCTGC | 2435 | TAATCTACTAAGCCTC | 2520 | TCCTCCATAGGTCAGC |
| 2351 | CTACCTGCATTGAGAC | 2436 | GTACCATCAGAAGCCC | 2521 | ATATGGATGCTACCTC |
| 2352 | GGCATAGTTACCACAC | 2437 | GGCATACCCTCATTAT | 2522 | GGATGCTACCTCTGTT |
| 2353 | GCCATGTCCTAGACTG | 2438 | GGTAATCCTCATTGAC | 2523 | GTCTAGGGTGGTCAAC |
| 2354 | GCCCTAGCAAGCCTTG | 2439 | GTAGCATCCAGGCATC | 2524 | CTTAAGATTGTGTCCC |
| 2355 | GGCTGACCTCCATAGA | 2440 | GGACCTATCTTTCAGC | 2525 | GATAGAAGTCCCTGAC |
| 2356 | GCTGACCTCCATAGAT | 2441 | GACATCTGCTAGAGGC | 2526 | TAGTTACCACACCAGT |
| 2357 | GTGCTACCCAACCTTG | 2442 | TCATGGTCCTAAGTGG | 2527 | GCACTATGCTCAACCT |
| 2358 | GCATTACAACCTCCTA | 2443 | GCTGCCCTAGATCCAA | 2528 | AGTGTGCCAATCACCC |
| 2359 | GTTGTACCTACTAAGC | 2444 | GGTCCTACTCCATGTA | 2529 | CAGATAAGTGCCCTAT |
| 2360 | ATGAGTATCACTACCC | 2445 | GATGCCTGACTAAGTG | 2530 | GACTGGAGCATTAAAC |
| 2361 | CAGCATGATAGGGTCC | 2446 | TAGTCAAGAACCAACC | 2531 | GGTAAACCCATAACTC |
| 2362 | TAGGACTGACCATTCA | 2447 | TAAGCCTTTGACCAGC | 2532 | ACTTCAGTAGGAACCC |
| 2363 | GCATGGTCCCTAACAA | 2448 | CTAACAAGGGTCTAGC | 2533 | GCATATCCACTGATCA |
| 2364 | GCATAGTTACCACACC | 2449 | TAGCCTCATACTGACC | 2534 | CAATTGTCAAAGGGCC |
| 2365 | TTAGGCAACCCAGCAC | 2450 | GCCTGTAAGCATATAC | 2535 | GTCTGGTTAGGTATGA |
| 2366 | GCAAGTCCATACACTC | 2451 | GGATGTACTTAGGCAC | 2536 | GTGGATTCTCCCTAGT |
| 2367 | AGCCTGTCCATTGATC | 2452 | GCTGAGACTTACCCTC | 2537 | ATCCTGATTGAGACCC |
| 2368 | TAGGCTGACCTCCATA | 2453 | GCCCATATCTACCTGG | 2538 | GACAATGCAGACCCTA |
| 2369 | AGAGTGGACCTAATCC | 2454 | AGTTGAGGGCAGTACC | 2539 | ACCCTAGTGTCATTTG |
| 2370 | GTACCTACTAAGCCAA | 2455 | CAATGGGTGTAGGTTC | 2540 | CCTATCAGAGGTGTGA |
| 2371 | GACATCATATTAGCCC | 2456 | GTTACCCTTAGCCCAG | 2541 | TGCATAATGACCTGAC |
| 2372 | CTGGTTAGGTCTATAG | 2457 | GCCCATATCTACCTGG | 2542 | GCAGCCCTTGTAGCTT |
| 2373 | CCTCTCATAGGATGCC | 2458 | GTGACTTTGGTGGGCC | 2543 | GGATCTTGCCCATCAC |
| 2374 | GTGCCAAGCTATCATA | 2459 | CTACTTACACTGAGGG | 2544 | GTGCTACCTGACAAGA |
| 2375 | TACCTACTAAGCCAAG | 2460 | GTTCATGTACCACAGG | 2545 | TTAGCCCAGTAATCAC |
| 2376 | TTGGGTGATTAGGGCC | 2461 | TGCTACTAACCAGCCC | 2546 | CTAGGGACTCAAATGC |
| 2377 | GATACAGACTGCTAAC | 2462 | CTTGTATGGCACCTAT | 2547 | GCCTACAGTTAGATCT |
| 2378 | GTAATGCCTATCAAGC | 2463 | GTAACCAGTACCAGGA | 2548 | GAATGTATCAAGCCTC |
| 2379 | TGTATTGGCACCCAGA | 2464 | GCCACTAGATAAGGTC | 2549 | GACTTGCCACTGAACC |
| 2380 | GCAAGTTACCCTCCAC | 2465 | GACAGTGAGCCAGTAC | 2550 | TAGCCAGAGTGTAGAT |
| 2381 | GCCTGAGCACTTCTAG | 2466 | TTTACATAGGAGGGCC | 2551 | GATGAGTATCACTACC |
| 2382 | GCACTAAGGACTGTTC | 2467 | TGTTGTACCTACTAAG | 2552 | GAGACCCTTAGAATCC |
| 2383 | ACCTACTAAGCCAAGC | 2468 | ACTAGTCCAGTCAAGG | 2553 | CAAGTAATGGTGTGCA |
| 2384 | GAAGGTTACTCCAGTT | 2469 | ATTGTCAGCTAGGAGC | 2554 | GCCCTAGAATGTTGAC |
| 2385 | GCTCTAAGACCTTGTC | 2470 | GAGCAATCTCCCTTAC | 2555 | GATGTACCTAGAAGGC |
| 2386 | GACCTCAACTTGTGCA | 2471 | GCTAGTCAAGAACCAA | 2556 | GAGACTTGATAAGCCC |
| 2387 | AGGCTACCAGACCCTT | 2472 | GACCAAGTCCATGAGC | 2557 | GCCAGACTTAGATGCT |
| 2388 | AATACACCAGCTTGCC | 2473 | ACTAACAAGGGTCTAG | 2558 | CTAGGATGCTTCAGGT |
| 2389 | GCCAAGCTATCATAAC | 2474 | TAGTGTGGAGTCATGG | 2559 | CAGCCCTTGTAGCTTA |
| 2390 | AGTACTCCCAACAGGC | 2475 | GGACTTGCCAATATGA | 2560 | TTGGATGTACTTAGGC |
| 2391 | GGACCTCAACTTGTGC | 2476 | TTGTGTAAGGGCTTGG | 2561 | GTGAACCCTCTAATGA |
| 2392 | GAGAGTGGACCTAATC | 2477 | TGCCACTAGATAAGGT | 2562 | GGATTGGGCAACTGTT |
| 2393 | ACCCTTAGACAACCTC | 2478 | GATGTAGACCCAGACC | 2563 | GAACCAACCAATAGGC |
| 2394 | AGTTGCCACTTGAGAC | 2479 | GTATACCTTGCCAGCA | | |
| 2395 | CTAGGACTGACCATTC | 2480 | GTCAGAATGCCTAGAC | | |

TABLE 35
Sequences of primers and PUFINs

| lncRNA | Primers |
|---|---|
| PUFIN2 | Forward primer1: |
| | 5′-ATGGCCCGTGTCCTATCTATTG-3′ |
| | Reverse primer1: |
| | 5′-TTTGGGTGCTTGGGGTTTTG-3′ |

TABLE 46
IPF risk variants associated with PUFIN1 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs13052134 | A | G | rs220224 | A | G | rs150794 | C | G |
| rs1628526 | A | G | rs138371252 | A | G | rs73905510 | T | C |
| rs399856 | A | G | rs62215061 | T | C | rs62215088 | T | C |
| rs399888 | A | G | rs220229 | T | C | rs150795 | A | G |
| rs399907 | A | G | rs62215062 | A | G | rs143257524 | A | G |
| rs118152395 | T | G | rs466709 | A | G | rs8128318 | T | G |
| rs1788355 | T | G | rs458769 | C | G | rs73905511 | A | G |
| rs527288390 | A | G | rs12627136 | T | C | rs62215089 | C | G |
| rs1054526 | T | C | rs141433941 | T | C | rs62215090 | T | G |
| 21:43431830 | T | C | rs56386303 | T | C | rs11203183 | C | G |
| rs7278794 | T | C | rs220231 | T | C | rs62215091 | T | C |
| rs7280167 | A | G | rs116862349 | A | T | rs2839451 | A | G |
| rs141008972 | T | C | rs468866 | A | G | rs150796 | A | G |
| rs11701177 | T | C | rs466848 | A | G | rs73369545 | A | G |
| rs11701229 | A | C | rs2839441 | T | C | rs117084275 | A | G |
| rs9984999 | T | C | rs113638625 | A | G | rs118038513 | A | T |
| rs117440974 | A | G | rs78643935 | A | C | rs117654961 | A | C |
| rs142183780 | T | C | rs2839442 | C | G | rs73369548 | T | C |
| rs220195 | T | C | rs220232 | T | C | rs73369551 | A | G |
| rs9981167 | T | C | rs62215064 | T | C | rs74623856 | T | C |
| rs9974393 | A | G | rs2839443 | T | C | rs116090522 | C | G |
| rs220196 | T | C | rs76537889 | T | C | rs118167181 | T | C |
| rs2839436 | T | C | rs112750029 | T | C | rs117241927 | A | G |
| rs2839437 | T | C | rs111767220 | T | C | rs117467290 | A | G |
| rs77687831 | T | C | rs220233 | T | C | rs116853111 | T | C |
| rs115791637 | A | G | rs220234 | A | G | rs117139501 | T | C |
| rs28378451 | T | C | rs55930257 | A | G | rs117441331 | A | G |
| rs220197 | A | G | rs220235 | T | C | rs60469491 | A | C |
| rs75601502 | A | G | rs220236 | A | C | rs150797 | C | G |
| rs116456054 | A | G | rs55955802 | T | C | rs60148749 | A | G |
| rs115758864 | T | C | rs1788356 | T | C | rs78248586 | T | C |
| rs220198 | A | C | rs1671820 | A | G | rs73905514 | T | C |
| rs220199 | A | G | rs220237 | T | C | rs2839452 | T | C |
| rs220200 | A | G | rs2839444 | T | C | rs2839453 | T | C |
| rs2839438 | A | G | rs220238 | A | G | rs73223503 | T | C |
| rs220201 | T | G | rs220239 | T | C | rs118042502 | A | C |
| rs220202 | T | C | rs2839445 | A | G | rs117770514 | A | G |
| rs6586293 | A | G | rs394068 | T | C | rs117920145 | A | G |
| rs220203 | A | G | rs420783 | T | C | rs147808669 | A | G |
| rs220204 | T | G | rs2839446 | T | C | rs117321106 | T | C |
| rs220205 | A | G | rs113922787 | T | C | rs35357179 | T | G |
| rs8127168 | C | G | rs2839447 | C | G | rs117098964 | A | G |
| rs220206 | C | G | rs75179071 | A | G | rs117506870 | A | T |
| rs2839439 | A | G | rs75596907 | A | G | rs118166356 | T | C |
| rs220208 | T | C | rs8132061 | T | C | rs2839454 | A | T |
| rs117995466 | A | C | rs12482564 | A | G | rs2839455 | T | C |
| rs220209 | A | G | rs76662477 | A | G | rs2839456 | T | G |
| rs220210 | T | C | rs75056445 | T | C | rs2839457 | T | C |
| rs117245917 | A | C | rs220240 | A | G | rs13050478 | T | G |
| rs220211 | A | G | rs220241 | A | G | rs73905515 | T | G |
| rs117008556 | A | G | rs220242 | A | C | rs7279867 | T | C |
| rs220212 | T | C | rs457186 | T | C | rs150798 | A | G |
| rs220213 | T | C | rs56003408 | A | G | rs150799 | A | G |
| rs220215 | T | C | rs139405122 | A | C | rs118133708 | T | C |
| rs7276454 | T | C | rs79280913 | T | C | rs2839458 | A | G |
| rs11701616 | A | G | rs9985048 | T | G | rs150800 | A | G |
| rs11701203 | A | G | rs9978701 | A | T | rs1869770 | T | C |
| rs73367318 | A | G | rs9982783 | T | C | rs73369571 | T | C |
| rs220216 | T | G | rs9985014 | T | C | rs73369574 | A | T |
| rs11701770 | A | G | rs9753864 | A | G | rs181803292 | A | G |
| rs461207 | A | G | rs220243 | A | C | rs13047154 | A | C |
| rs75419238 | C | G | rs28487289 | T | C | rs117770500 | A | C |
| rs220217 | T | C | rs429870 | A | G | rs13051807 | A | G |
| rs17114159 | T | C | rs446831 | A | G | rs220247 | T | C |
| rs11701930 | A | G | rs117985031 | T | C | rs220248 | A | G |
| rs11203180 | A | G | rs2839448 | A | C | rs2839459 | T | C |
| rs11203181 | A | C | rs4920059 | T | C | rs2298688 | C | G |
| rs11203182 | A | G | rs73369515 | A | G | rs145721751 | A | G |
| rs7275759 | A | G | rs220245 | T | C | rs7281803 | A | G |
| rs118131263 | T | C | rs218372 | T | C | rs220249 | A | G |
| rs220218 | T | C | rs73221497 | T | G | rs220250 | T | C |
| 21:43443266 | A | C | rs56400714 | A | G | rs59203781 | T | C |
| rs144190050 | A | G | rs2839449 | T | C | rs220251 | A | G |
| rs62215060 | A | G | rs78494579 | T | C | rs220252 | A | G |
| rs2839440 | T | C | rs220246 | A | G | rs60818033 | C | G |
| rs220219 | C | G | rs12482228 | T | C | rs9981939 | A | C |
| rs220220 | T | C | rs73221500 | T | C | rs118058040 | T | C |
| rs3737429 | T | C | rs150790 | A | G | rs57215902 | A | G |
| rs11701948 | A | G | rs73221502 | T | C | rs59178061 | A | T |
| rs73221486 | A | C | rs35756574 | C | G | rs117456646 | A | G |
| rs67858562 | T | C | rs117635456 | A | T | rs117196041 | T | C |
| rs75967781 | A | C | rs150791 | A | G | rs462098 | C | G |
| 21:43446546 | T | G | rs150792 | A | G | | | |
| rs77898845 | A | G | rs150793 | T | C | | | |
| rs220223 | A | G | rs5024160 | A | T | | | |

TABLE 47
IPF risk variants associated with PUFIN2 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs78068165 | A | G | rs76145101 | A | G | rs1505279 | T | C |
| rs118180572 | A | T | rs67488148 | A | G | rs1505280 | A | G |
| rs4302005 | T | C | rs12440794 | A | G | rs56092237 | T | C |
| rs117911810 | A | G | rs11630372 | T | G | rs55949616 | A | C |
| rs62006290 | T | G | rs2411238 | T | C | rs2119402 | T | C |
| rs2624268 | T | C | rs2411239 | A | T | rs969564 | T | C |
| rs2725795 | T | C | rs59730940 | A | G | rs72722909 | A | T |
| rs7173968 | T | C | rs12442494 | A | C | rs140298961 | T | C |
| rs2624271 | A | G | rs10152813 | A | T | rs138033972 | A | G |
| rs2725794 | C | G | rs7179611 | C | G | rs156791 | A | G |
| rs10152568 | T | C | rs1380939 | T | C | rs79073092 | T | C |
| rs77881985 | A | G | rs1304827 | T | C | rs156790 | A | T |
| rs77468159 | A | G | rs1460798 | C | G | rs62006048 | T | C |
| rs61480073 | A | C | rs1460799 | T | C | rs156657 | T | C |
| rs4395048 | T | C | rs1460800 | A | G | rs72722912 | C | G |
| rs4258586 | T | C | rs79957485 | A | G | rs117754122 | C | G |
| rs12442819 | T | G | rs12443075 | T | C | rs150032908 | T | C |
| rs11073358 | T | G | rs7179980 | A | T | rs11070177 | T | C |
| rs4924306 | C | G | rs11639006 | A | C | rs156789 | T | C |
| rs9646220 | T | C | rs1821380 | C | G | rs11070178 | A | G |
| rs11858412 | A | T | rs2019469 | A | T | rs17613185 | T | C |
| rs28601720 | C | G | rs971203 | A | T | rs142440594 | T | C |
| rs72733127 | T | C | rs12907914 | C | G | rs17691453 | T | C |
| rs4924307 | A | C | rs973932 | C | G | rs12917528 | C | G |
| rs1023642 | A | G | rs973931 | C | G | rs11853050 | T | C |
| rs11855792 | T | C | rs7175659 | A | G | rs156783 | T | G |
| rs17660283 | T | C | rs9888720 | T | C | rs13329154 | T | C |
| rs11636042 | A | C | rs9919961 | A | G | rs156784 | A | G |
| rs11632328 | T | C | rs74697980 | C | G | rs12904023 | A | G |
| rs11632437 | T | C | rs34278069 | T | C | rs35201540 | T | C |
| rs1055725 | T | C | rs62006470 | A | G | rs17691608 | A | G |
| rs7167179 | A | G | rs1460794 | T | C | rs1847663 | A | G |
| rs72733132 | A | G | rs11853413 | A | G | rs72722918 | C | G |
| rs4417528 | A | C | rs4244568 | T | G | 15:39546528 | C | G |
| rs4429265 | T | C | rs11070169 | T | C | rs12915271 | T | C |
| rs4614698 | A | G | rs76940068 | T | C | rs156785 | A | G |
| rs72733134 | T | C | rs117538431 | A | G | rs60456859 | A | C |
| rs72733135 | T | C | rs11633975 | A | G | rs156786 | A | G |
| rs983743 | T | C | rs983482 | A | G | rs156787 | A | G |
| rs12916564 | A | C | rs10851378 | A | G | rs79514526 | A | G |
| rs77115844 | T | C | rs36098877 | T | C | rs11636461 | C | G |
| rs76850379 | C | G | rs74007714 | T | C | rs62008264 | A | C |
| rs7177787 | A | G | rs72737196 | A | G | rs145222516 | T | C |
| rs10852031 | A | G | rs10851379 | A | C | 15:39552068 | A | T |
| rs11853624 | A | G | rs11070170 | A | C | rs77470370 | A | G |
| rs11632791 | A | G | rs28425192 | A | G | rs72722921 | T | C |
| rs79840553 | A | G | rs11638316 | A | C | rs34566881 | T | C |
| rs75276658 | C | G | rs78547831 | T | C | rs138417060 | A | G |
| rs72733142 | A | C | rs112595174 | A | G | rs156805 | A | G |
| rs34225173 | A | G | rs1841438 | A | T | rs4392035 | A | G |
| rs12102059 | A | C | rs1841437 | A | G | rs75524703 | A | G |
| rs72733146 | T | C | rs62006471 | A | G | rs4509989 | T | C |
| rs17660474 | T | C | rs117564456 | A | G | rs116927841 | T | G |
| rs72733147 | T | C | rs143227939 | T | C | 15:39556231 | T | C |
| rs72733148 | T | C | rs11637986 | T | C | rs17691799 | A | G |
| rs111928892 | T | C | rs11070171 | T | C | rs276865 | A | G |
| rs76754134 | A | G | rs62006472 | A | G | rs16968570 | C | G |
| rs72733149 | T | C | rs7181384 | A | G | rs1505272 | T | C |
| rs9920784 | A | G | rs151025531 | A | G | rs79098946 | A | G |
| rs76719447 | A | T | rs2004175 | T | G | rs57303613 | A | G |
| rs4924308 | T | G | rs62006474 | A | C | rs59469479 | T | C |
| rs76065080 | T | G | rs1350747 | T | C | rs11854792 | A | G |
| rs80185266 | A | T | rs17605994 | T | C | rs12907606 | T | C |
| rs6496013 | T | C | rs62006487 | A | G | rs28557171 | A | T |
| rs6496014 | T | C | rs72737199 | T | C | rs28460795 | A | G |
| rs17660503 | A | G | rs62006488 | T | C | rs12442944 | A | G |
| rs7176726 | A | G | rs72737201 | T | C | rs12442876 | A | T |
| rs16967640 | T | C | rs4608312 | A | G | rs12442955 | T | C |
| rs4555128 | C | G | rs144054460 | A | G | rs12442932 | A | G |
| rs9920338 | A | G | rs12101778 | T | C | rs12440113 | T | C |
| rs148668510 | T | C | rs78379000 | C | G | rs77660997 | A | G |
| rs144380159 | A | G | rs78028880 | T | C | rs12905527 | T | C |
| rs147818212 | A | C | rs10851380 | A | G | rs62008266 | A | C |
| rs72733151 | C | G | rs113090061 | A | G | rs112833594 | T | C |
| rs141288153 | A | T | rs11070172 | T | C | rs11631371 | T | G |
| rs72733152 | T | C | rs59402905 | T | C | rs6492866 | A | G |
| rs72733154 | A | G | rs9806216 | T | C | rs72722933 | T | C |
| rs72733156 | A | G | rs7168805 | A | G | rs11634150 | A | G |
| rs72733157 | C | G | rs151072381 | A | G | rs1354597 | T | C |
| rs77112240 | C | G | rs4924326 | C | G | rs11637020 | A | C |
| rs72733159 | A | G | rs77845626 | A | C | rs11070182 | T | C |
| rs16967641 | C | G | rs10152803 | A | G | rs66504119 | C | G |
| rs8040465 | T | C | rs10152856 | T | C | rs147544050 | A | T |
| rs74771640 | T | C | rs72739304 | A | G | rs117363831 | T | C |
| rs17660586 | A | G | rs113807095 | A | G | rs8043034 | A | C |
| rs12594565 | A | G | rs78539969 | A | G | rs1371968 | T | G |
| rs76428393 | A | T | rs4924327 | A | G | rs6492867 | T | C |
| rs12594575 | A | G | rs4924328 | T | C | rs55815708 | A | G |
| rs17602752 | T | G | rs10520115 | A | G | rs12591462 | T | C |
| rs138312566 | T | C | rs56905145 | A | G | rs79925924 | A | G |
| rs11073360 | T | G | rs35151883 | T | G | rs4586401 | A | G |
| rs56152793 | A | G | rs117463733 | T | C | rs145172346 | A | G |
| rs10852032 | A | G | rs141094873 | T | C | rs112821124 | A | G |
| rs7496821 | A | G | rs880716 | A | T | rs35296796 | A | G |
| rs11629503 | A | G | rs750016 | T | G | rs139801643 | A | G |
| rs11636249 | T | C | rs1585452 | T | G | rs76221593 | T | C |
| rs7178589 | A | G | rs74394790 | T | C | rs149641937 | A | G |
| rs7178596 | A | G | rs137991046 | T | C | rs34356989 | T | C |
| rs17660736 | T | C | rs1460801 | A | C | rs12595668 | A | G |
| rs7181114 | C | G | rs79286965 | C | G | rs77252089 | T | G |
| rs55813105 | T | C | rs35873006 | C | G | rs2135146 | A | C |
| rs55774654 | T | C | rs79018971 | C | G | rs1157997 | T | C |
| rs7183083 | A | T | rs17680281 | A | G | rs6492868 | A | G |
| rs56138388 | A | T | rs1368812 | T | C | rs1157998 | T | C |
| rs79421485 | A | G | rs77537015 | T | C | rs1157999 | A | C |
| rs7183486 | A | T | rs1368813 | A | C | rs746265 | A | C |
| rs7181891 | T | G | rs1368814 | A | G | rs7178689 | T | C |
| 15:39179918 | A | C | rs1368815 | T | C | rs17614000 | T | C |
| rs138514699 | T | C | rs76262680 | A | C | rs7179853 | T | C |
| rs16967690 | A | G | rs4344714 | A | G | rs17614032 | A | T |
| rs117287392 | A | C | rs16968030 | A | T | rs34947582 | A | C |
| rs16967692 | T | C | rs117017809 | T | C | 15:39577879 | A | C |
| rs12594929 | C | G | rs990579 | A | G | rs78736494 | A | C |
| rs12594953 | A | G | rs8034272 | T | C | rs28630993 | T | C |
| rs12592767 | A | T | rs8034477 | T | C | rs17692246 | T | C |
| rs12593309 | T | C | rs8039377 | A | G | rs17692294 | A | G |
| rs12592792 | A | G | rs76502540 | A | G | rs16968662 | A | G |
| rs12592794 | A | G | rs7163993 | T | C | rs79568057 | A | G |
| rs35026885 | C | G | rs7164452 | A | G | rs11631702 | A | G |
| rs16967700 | A | G | rs7180398 | A | T | rs117159976 | T | C |
| rs4628936 | T | C | rs7180932 | A | C | rs117959877 | T | C |
| rs4477676 | A | C | rs12595806 | A | G | rs11070183 | T | C |
| rs1836169 | A | G | rs7180955 | T | C | rs142772826 | T | C |
| rs4310828 | A | G | rs7182405 | T | G | rs8037045 | C | G |
| rs1836170 | A | G | 15:39361986 | T | C | rs8037961 | T | C |
| rs12443342 | C | G | rs2117208 | T | C | rs6492869 | A | T |
| rs4541050 | A | G | rs55849520 | A | G | rs6492870 | C | G |
| rs2411219 | T | C | rs7171196 | T | C | rs10152469 | T | G |
| rs34053185 | T | G | rs7171382 | T | C | rs28397054 | T | C |
| rs62006314 | A | G | rs7171236 | A | C | rs4454943 | C | G |
| rs7172988 | A | G | rs1824491 | A | T | rs7173445 | T | C |
| rs7177997 | A | C | rs8037274 | T | C | rs7172230 | A | C |
| rs75342815 | A | C | rs4923815 | A | C | rs7174033 | T | C |
| rs12901090 | A | T | rs4924330 | A | G | rs12909502 | A | G |
| rs77932841 | T | G | rs4923816 | T | C | rs76894162 | C | G |
| 15:39188204 | T | C | rs4924331 | A | G | rs75737303 | T | C |
| rs11852946 | A | C | rs17607365 | T | C | rs76814026 | A | G |
| rs79340828 | T | C | rs6492855 | A | G | rs2119406 | T | C |
| rs75072142 | A | G | rs6492856 | A | G | rs77793894 | T | G |
| rs79818199 | T | C | rs115101834 | T | C | rs149735900 | A | G |
| rs77558024 | T | C | rs34565403 | A | G | rs6492871 | T | C |
| rs2411220 | T | C | rs12900493 | A | G | rs8038768 | A | G |
| rs77455143 | T | C | rs7162018 | A | G | rs6492872 | T | C |
| rs6496015 | A | G | rs77240555 | A | C | rs75378913 | T | G |
| rs76422111 | T | C | rs12900637 | A | C | rs116855688 | T | C |
| rs16967718 | C | G | rs7166929 | A | C | rs74599156 | T | C |
| rs1897737 | T | C | rs16968084 | T | C | rs11070184 | T | C |
| rs147068210 | C | G | rs7167457 | A | G | rs79214604 | T | C |
| rs78048698 | A | G | rs7167357 | T | C | rs16968670 | T | G |
| rs112795779 | T | C | rs7167354 | A | G | rs2196973 | C | G |
| rs1319670 | T | C | rs59904802 | A | G | rs72722947 | C | G |
| rs11852439 | A | G | rs16968091 | T | C | rs78396433 | T | C |
| rs75180414 | T | C | rs79635731 | T | C | rs16968678 | T | C |
| rs77222091 | T | C | rs4924332 | T | C | rs7168589 | C | G |
| rs74371785 | T | C | rs4924333 | A | G | rs7169101 | T | C |
| rs4923807 | T | C | rs4924334 | A | C | rs77123576 | A | T |
| rs80170723 | A | G | rs78287049 | A | T | rs78399646 | A | G |
| rs76236876 | A | T | 15:39367052 | A | C | rs62002276 | T | C |
| rs76618764 | T | C | rs62006496 | T | C | rs75795406 | T | C |
| rs79619835 | C | G | rs71403557 | A | G | rs62002277 | C | G |
| rs76946079 | A | C | rs12915964 | T | C | rs1394750 | A | G |
| rs79110517 | C | G | rs12595617 | A | G | rs74597454 | T | G |
| rs11632536 | T | C | rs12915961 | A | G | rs75638406 | C | G |
| rs11073362 | A | G | rs72739316 | A | T | rs76756124 | T | C |
| rs193000795 | A | G | rs12595675 | T | C | rs34799287 | T | C |
| rs1821383 | A | G | rs16968099 | T | C | rs74716084 | A | G |
| rs112906631 | T | C | rs7180973 | A | G | rs17692507 | A | G |
| rs28468472 | A | G | rs7180698 | A | G | rs77317459 | T | C |
| rs12443217 | T | C | rs12595747 | T | C | rs2053445 | C | G |
| rs2060369 | A | G | rs12595850 | C | G | rs17692567 | A | C |
| rs4343249 | A | G | rs12591588 | A | G | rs78932148 | A | G |
| rs7180133 | A | G | rs7169980 | A | C | rs57115092 | A | G |
| rs11853822 | T | C | rs79405725 | A | G | rs12907993 | A | G |
| rs1368808 | A | G | rs12591177 | T | C | rs11633545 | T | G |
| rs1368809 | T | C | rs17681215 | A | G | rs111521641 | T | C |
| rs8030435 | A | G | rs2100784 | T | C | rs76595690 | T | C |
| rs147611118 | T | C | rs2411240 | T | C | rs74244653 | A | G |
| rs10468093 | T | C | rs2411241 | A | G | rs77971865 | T | G |
| rs12439653 | T | C | rs16968115 | A | C | rs77492259 | A | G |
| rs12908361 | A | G | rs7166614 | T | G | rs78388737 | C | G |
| rs7175727 | C | G | rs16968121 | A | C | rs79921152 | A | G |
| rs1433894 | C | G | rs16968124 | T | C | rs74824754 | T | C |
| rs150223302 | T | C | rs7165302 | A | G | rs79350006 | A | T |
| rs7168832 | C | G | rs186780393 | A | C | rs12232252 | T | G |
| rs12440635 | A | T | rs11070173 | C | G | rs12232251 | T | C |
| rs12439991 | A | G | rs77174350 | A | G | rs116929653 | T | C |
| rs1946636 | A | G | rs2411242 | T | C | rs2271064 | A | G |
| rs141137277 | T | C | rs114721875 | A | G | rs12232322 | T | C |
| rs139111719 | A | C | rs2411243 | A | G | rs11631253 | A | T |
| rs28790611 | A | G | rs2411244 | T | G | rs74708443 | T | C |
| rs2411214 | T | C | rs2411245 | A | T | rs16968711 | A | G |
| rs1114247 | A | T | rs1460787 | T | C | rs80302897 | T | G |
| rs9972583 | T | C | rs76414176 | T | G | rs75212645 | T | G |
| rs28715410 | C | G | rs77351932 | A | G | rs28557453 | C | G |
| rs72733179 | C | G | rs12909296 | T | C | rs60035130 | A | T |
| rs113021992 | T | G | rs12372998 | T | C | rs79658763 | A | G |
| rs35538929 | A | G | rs12372999 | T | C | rs62002280 | T | C |
| rs116949350 | T | C | rs7173406 | C | G | rs62002281 | A | T |
| rs149003200 | A | C | rs62006517 | T | C | rs7497593 | T | C |
| rs724234 | A | G | 15:39372623 | A | G | rs62002282 | A | G |
| rs16967763 | T | C | 15:39372766 | A | G | rs62002283 | A | G |
| rs11853238 | A | T | rs8024778 | A | T | rs10152323 | T | C |
| rs78968044 | T | C | rs55658781 | T | C | rs9302101 | A | G |
| rs7163517 | T | C | rs716980 | T | C | rs59203590 | A | G |
| rs7162298 | A | G | rs77466127 | A | C | rs77609868 | T | C |
| rs892405 | A | C | rs12148236 | A | G | rs62002285 | C | G |
| rs117508131 | A | T | rs75357312 | T | C | rs7171648 | T | C |
| rs4410046 | A | G | rs117228223 | A | G | rs3892721 | T | G |
| rs731223 | T | C | rs117561334 | T | C | rs3892720 | T | C |
| rs16967765 | A | C | rs6492857 | T | C | rs3850778 | C | G |
| rs17661398 | A | T | rs6492858 | A | G | rs182231911 | T | G |
| rs11855813 | T | C | rs4628937 | T | C | rs72489206 | T | G |
| rs12914368 | A | C | rs4628938 | T | G | rs72722956 | A | G |
| rs79346257 | A | G | rs147656202 | A | C | rs1371964 | A | G |
| rs57332488 | A | T | rs80191201 | C | G | rs8036794 | A | T |
| rs60989439 | T | C | rs72739328 | A | G | rs1371963 | A | G |
| rs55660668 | C | G | rs8026370 | T | C | rs8041440 | A | C |
| rs56025640 | A | G | rs77240930 | A | G | rs77599564 | T | G |
| rs117111337 | A | G | rs12591981 | T | C | rs72722959 | A | G |
| rs34798757 | T | C | rs62006522 | A | G | rs66497604 | T | C |
| rs79137672 | A | G | rs2035065 | T | C | rs4328411 | T | C |
| rs116895890 | T | C | rs959863 | A | G | rs4429266 | A | G |
| rs12591604 | A | T | rs55987810 | T | C | rs4506868 | A | G |
| rs28558859 | C | G | rs80218661 | A | G | rs4260028 | T | C |
| rs12438882 | T | C | rs74870145 | A | T | rs3973350 | T | C |
| rs139540151 | A | G | rs56111333 | A | G | rs56214701 | T | G |
| rs146056042 | A | G | rs147250766 | C | G | rs8027538 | T | C |
| rs79984442 | A | T | rs6492861 | C | G | rs118081226 | A | G |
| rs78824515 | A | G | rs8033196 | A | G | rs8028438 | T | C |
| rs7181150 | T | C | rs1841441 | T | C | rs8028483 | C | G |
| rs117923554 | A | G | rs1841440 | A | T | rs117552204 | T | C |
| rs11070160 | A | G | rs1870657 | A | G | rs7167869 | C | G |
| rs10851370 | T | G | rs985452 | A | T | rs1530219 | A | G |
| rs11858981 | T | C | rs985451 | T | C | rs145943589 | A | G |
| rs8027106 | T | C | rs4924335 | A | G | rs10152992 | A | G |
| rs8026059 | A | C | rs4924336 | A | G | rs8041286 | A | G |
| rs8026341 | A | G | rs76484045 | T | C | rs16968739 | A | G |
| rs28435270 | T | C | rs2411247 | A | G | rs3862144 | T | C |
| rs9972543 | A | G | rs11630786 | T | C | rs3862145 | T | C |
| rs8037567 | A | G | rs78072829 | A | G | rs994612 | T | C |
| rs9972580 | T | C | rs7164957 | A | G | rs994613 | T | C |
| rs8037708 | A | G | rs7170532 | T | C | rs7164730 | T | C |
| rs28465416 | A | G | rs35258758 | A | G | rs4471651 | A | G |
| rs4924310 | T | G | rs34769210 | A | G | rs4622486 | A | C |
| rs60795407 | A | G | rs57745834 | T | G | rs8032190 | T | C |
| rs9972315 | A | G | rs56242331 | T | C | rs8031007 | T | C |
| rs77636544 | A | G | rs35130744 | A | G | rs8031142 | T | C |
| rs28834398 | C | G | rs36107824 | T | C | rs2868072 | A | G |
| rs117046709 | T | G | rs71470586 | A | G | rs997752 | T | C |
| rs1118028 | T | C | rs990536 | T | C | rs998005 | T | C |
| rs1118027 | A | G | rs7183660 | T | C | rs1354596 | A | T |
| rs1960601 | T | C | rs2068288 | A | C | rs139104748 | A | G |
| rs12911429 | A | T | rs16968128 | A | T | rs116947915 | A | G |
| rs2411216 | A | G | rs16968130 | T | C | rs2033938 | T | C |
| rs8039458 | A | T | rs12910546 | T | C | rs16968751 | T | C |
| rs8039460 | A | G | rs35483536 | A | G | rs2033937 | A | G |
| rs144889540 | T | C | rs73397662 | T | G | rs76608188 | A | C |
| rs8041326 | T | C | rs117665137 | T | C | rs894043 | A | G |
| rs7497579 | A | G | rs58417396 | T | C | rs894042 | A | G |
| rs78933758 | T | C | rs73397664 | T | C | rs992932 | A | T |
| rs28865003 | T | C | rs12915083 | A | C | rs1857792 | T | C |
| rs8036109 | A | G | rs16968139 | C | G | rs920506 | T | C |
| rs8035861 | C | G | rs71470588 | A | G | rs920505 | T | C |
| rs146728372 | T | C | rs16968141 | T | C | rs12905881 | A | C |
| rs8036186 | A | C | rs73397668 | T | C | rs59249266 | T | C |
| rs8036349 | A | C | rs11637989 | A | G | rs59764262 | T | C |
| rs8037838 | T | C | rs57389883 | A | G | rs7174914 | T | C |
| rs8036675 | A | C | rs36011093 | T | C | rs7173987 | C | G |
| rs28865009 | T | C | rs113246579 | A | G | rs7174004 | A | G |
| rs79725909 | A | G | rs16968143 | T | C | rs1899226 | A | T |
| rs28851524 | A | G | rs9972304 | T | C | rs10152848 | A | G |
| rs2411218 | A | G | rs34467867 | T | C | rs10152864 | C | G |
| rs11855699 | C | G | rs35064685 | A | G | rs8035243 | T | C |
| rs77777824 | T | G | rs74007753 | T | C | rs8035253 | A | C |
| rs3959231 | T | C | rs59771725 | A | G | rs1899225 | T | C |
| rs2411221 | T | C | rs2411246 | T | C | rs8041744 | A | T |
| rs2411222 | C | G | rs724729 | T | C | rs76235274 | T | C |
| rs35221528 | A | T | rs8034417 | A | G | rs11634774 | A | G |
| rs11856672 | T | C | rs71470589 | A | G | rs60312268 | T | C |
| rs75774801 | A | G | rs8035543 | A | G | rs62002309 | T | C |
| rs144114625 | A | G | rs113423750 | T | C | rs74771443 | A | T |
| rs12909862 | T | C | rs1841436 | C | G | rs141364926 | T | C |
| rs11857756 | A | G | rs149154271 | T | C | rs1505270 | T | C |
| rs28857283 | A | G | rs16968165 | T | G | rs28716672 | A | G |
| rs61501627 | T | C | rs1460793 | C | G | rs28508536 | T | C |
| rs7178881 | A | C | rs79563865 | T | C | rs28666897 | A | G |
| rs10851371 | C | G | rs1460791 | A | C | rs59912278 | A | G |
| rs75684959 | T | G | rs190622131 | A | G | rs56758724 | A | G |
| rs1594810 | A | C | rs74007755 | T | C | rs61648840 | A | T |
| rs58828841 | T | C | rs1460790 | T | C | rs28430572 | T | C |
| rs61620830 | A | G | rs1460789 | A | G | rs72722969 | A | G |
| rs117805056 | A | G | rs17608906 | A | G | rs76653295 | A | T |
| rs149642964 | T | C | rs7166411 | T | C | rs935051 | T | C |
| rs1368802 | T | C | rs1380934 | A | G | rs2411267 | T | C |
| rs1368801 | C | G | rs117522995 | A | T | rs75897752 | A | G |
| rs1368800 | C | G | rs16968186 | A | G | rs4277317 | T | C |
| rs1368799 | C | G | rs60976146 | T | C | rs4627311 | A | G |
| rs1368798 | A | G | rs73397702 | T | C | 15:39613803 | A | G |
| rs7497101 | T | C | 15:39404188 | A | T | rs140086489 | T | C |
| rs11854787 | C | G | rs73399703 | T | C | rs183375538 | A | G |
| rs1368797 | A | G | rs61212366 | T | C | rs56341217 | A | G |
| rs28635322 | T | C | rs73399704 | A | G | rs58587604 | T | G |
| rs1368796 | T | C | rs275230 | A | G | rs60380125 | A | G |
| rs1433887 | T | C | rs188005 | A | G | rs55851101 | T | C |
| rs1433886 | A | G | rs1078003 | A | G | rs55977125 | T | C |
| rs8027128 | T | C | rs73399709 | A | G | rs1371958 | A | G |
| rs8025766 | A | C | rs78165672 | A | G | rs1371959 | A | T |
| rs8026369 | A | G | rs12323962 | T | G | rs1371960 | T | C |
| rs8026102 | A | G | rs2219593 | C | G | rs1440800 | T | C |
| rs8026743 | A | G | rs2219592 | C | G | rs1978915 | A | C |
| rs8026750 | A | G | rs2199315 | T | C | rs73387421 | A | G |
| rs117830266 | A | G | rs73399712 | T | C | rs73387425 | C | G |
| rs28764177 | T | C | rs59882434 | T | G | rs72722970 | A | G |
| rs117855779 | T | C | rs275229 | A | C | rs16968766 | T | C |
| rs1821382 | T | C | rs275228 | A | G | rs57932457 | T | C |
| rs1836167 | C | G | rs2085823 | T | C | rs7170096 | C | G |
| rs59784184 | T | C | rs117538107 | A | G | rs76955089 | T | C |
| rs28820670 | A | G | rs142512885 | A | T | rs8031369 | C | G |
| rs28793588 | T | C | rs275227 | A | C | rs8035691 | A | G |
| rs139816989 | A | T | rs275226 | A | C | rs77944698 | T | G |
| rs71470580 | A | G | rs57493295 | A | G | rs186127820 | A | T |
| rs1433882 | A | G | rs59507768 | A | G | rs9944272 | A | T |
| rs28599693 | C | G | rs80340570 | T | C | rs77302526 | A | G |
| rs16967792 | A | G | rs115747213 | A | G | rs56003663 | T | C |
| rs7178890 | A | C | rs275225 | A | G | rs6492877 | A | G |
| rs1433881 | C | G | rs140000420 | T | G | rs72722972 | A | G |
| rs1433880 | T | G | rs113891117 | T | C | rs11636013 | T | C |
| rs1433879 | T | C | rs275223 | A | T | rs11631318 | T | C |
| rs1433878 | T | C | rs275222 | T | C | rs8032326 | A | C |
| rs75276651 | C | G | rs275221 | A | C | rs17621280 | T | C |
| rs12898413 | T | C | rs72739336 | A | C | rs6416433 | A | G |
| rs142319423 | T | C | rs188004 | A | G | rs8033007 | C | G |
| rs75816310 | A | G | rs275220 | T | C | rs74007800 | T | C |
| rs149271013 | T | C | rs112823786 | T | C | rs73387443 | T | C |
| rs117621153 | A | G | rs191792892 | T | C | rs73387445 | A | G |
| rs139626222 | C | G | rs275218 | A | T | rs59550961 | A | G |
| rs768269 | A | C | rs183341543 | A | G | rs17694064 | T | C |
| rs16967810 | T | G | rs73399744 | C | G | rs73387449 | A | G |
| rs77935481 | T | C | rs1870656 | A | G | rs17694082 | C | G |
| rs9989301 | A | T | rs73399745 | A | G | rs17694111 | T | C |
| rs1433877 | T | C | rs275217 | C | G | rs77693174 | T | G |
| rs111499479 | T | C | rs149505303 | A | G | rs77369538 | T | G |
| rs17661677 | A | T | rs75146277 | C | G | rs7167789 | A | T |
| rs1433876 | T | G | rs113502551 | A | C | rs56113415 | A | G |
| rs12904169 | A | G | rs275190 | A | T | rs11638016 | T | C |
| rs12905686 | T | G | rs74007759 | T | C | rs6492878 | T | G |
| rs4302006 | A | C | rs77139127 | T | C | rs7179941 | A | G |
| rs11853486 | T | C | rs79943956 | T | C | rs7181250 | T | C |
| rs146926009 | T | C | rs77558673 | A | G | rs6492879 | T | C |
| rs143517477 | A | G | rs11070174 | T | C | rs6492880 | T | C |
| rs7173055 | A | G | rs112405992 | A | G | rs12916873 | T | C |
| rs7173554 | A | C | rs2623468 | A | G | rs8024927 | T | C |
| rs7174950 | T | C | rs56165732 | T | G | rs10851382 | T | C |
| rs1433875 | A | G | rs60285256 | T | C | rs8025239 | A | G |
| rs148761446 | T | C | rs188397981 | T | C | rs10851383 | C | G |
| rs1036301 | A | G | rs275187 | C | G | rs2119403 | A | C |
| rs1036302 | T | G | rs78780618 | T | C | rs2119404 | T | C |
| rs4561440 | C | G | rs7359184 | T | C | rs74427884 | A | G |
| rs62006342 | C | G | rs77980869 | T | G | rs75684456 | T | C |
| rs1036303 | A | C | rs7359268 | A | G | rs72722976 | C | G |
| rs12102253 | A | T | rs275215 | A | G | rs17621729 | A | G |
| rs117007183 | T | C | rs56292980 | A | G | rs10851384 | A | G |
| rs1433895 | A | G | rs58467950 | T | C | rs11070185 | T | C |
| rs56323020 | C | G | rs55831150 | T | C | rs10851385 | A | G |
| rs4244566 | T | C | rs73399773 | T | C | rs11629938 | A | G |
| rs11630597 | C | G | rs16968218 | A | G | rs79387284 | T | C |
| rs4923809 | T | C | rs61685635 | A | T | rs10520128 | C | G |
| 15:39244786 | T | C | rs275213 | C | G | rs11070187 | A | C |
| 15:39244795 | A | T | rs275212 | T | C | rs11070188 | A | G |
| rs4283208 | A | G | rs275211 | T | C | rs11070189 | A | G |
| rs62006345 | A | G | rs73399783 | A | C | rs11631325 | A | G |
| rs8039562 | T | G | 15:39438489 | A | G | rs12591944 | A | C |
| rs8042298 | A | G | rs275203 | A | G | rs12592015 | A | G |
| rs4924311 | A | G | rs73399785 | T | C | rs11070190 | A | G |
| rs10851372 | A | G | rs79540806 | A | G | rs11070191 | A | G |
| rs10851373 | A | G | rs117227769 | T | C | rs11070192 | A | G |
| rs4924312 | T | C | rs78113786 | A | G | rs7174403 | T | C |
| rs142793800 | T | C | rs74523450 | A | G | rs7173531 | A | G |
| rs55869358 | C | G | rs72739341 | A | G | rs72722978 | T | C |
| rs116950017 | A | C | rs275199 | T | C | rs11070193 | T | C |
| rs9920380 | A | G | rs78599517 | A | G | rs79179908 | A | G |
| rs4924313 | A | G | rs76163926 | A | T | rs10851386 | T | C |
| rs4924314 | T | C | rs2816588 | T | C | rs62002318 | A | G |
| rs67653650 | A | T | rs79606349 | T | C | rs149753925 | T | C |
| rs67904742 | A | G | rs275184 | T | G | rs62002319 | T | C |
| rs967498 | T | C | rs275183 | A | G | rs9652437 | A | G |
| rs12906888 | A | G | rs77695349 | T | C | rs77493130 | T | C |
| rs118051931 | A | G | rs275182 | A | G | rs11632141 | T | C |
| rs12906667 | A | C | rs275181 | T | C | rs11637839 | T | C |
| 15:39250088 | A | T | rs275180 | A | C | rs11639391 | T | C |
| rs2117202 | C | G | rs275179 | A | G | rs71471500 | C | G |
| rs4924315 | T | G | rs275178 | T | C | rs7182018 | A | G |
| rs2117203 | C | G | rs75712825 | T | C | rs3913763 | T | C |
| rs12911780 | A | G | rs7169516 | A | C | rs10851387 | A | G |
| rs12911790 | A | G | rs275177 | T | C | rs2033939 | A | G |
| rs10400893 | T | C | rs73401709 | T | C | rs1080066 | A | G |
| rs2164006 | T | G | rs7172086 | T | C | rs1822105 | T | C |
| rs12593524 | C | G | rs7177128 | A | T | rs1440802 | T | C |
| rs66780974 | A | G | 15:39449803 | T | C | rs17694988 | T | C |
| rs149433097 | T | C | rs16968273 | A | G | rs16968876 | T | C |
| rs61341654 | A | G | rs35791759 | T | G | rs2119405 | T | C |
| rs7179864 | T | C | rs139857185 | A | G | rs8030163 | C | G |
| rs7179209 | A | G | rs62006528 | A | G | rs117193619 | T | C |
| rs1158417 | T | C | rs74007771 | A | T | rs6492881 | C | G |
| rs4924317 | A | G | rs191785614 | T | G | rs1440803 | T | C |
| rs1368803 | T | C | rs55857431 | T | G | rs7180177 | C | G |
| rs1368804 | C | G | rs4290448 | T | C | rs116989441 | T | C |
| rs1368805 | A | T | rs2170408 | T | C | rs7181922 | T | C |
| rs11853815 | A | C | rs56161634 | T | C | rs12917333 | C | G |
| rs1120895 | T | C | rs56021915 | A | G | rs16968890 | C | G |
| rs9788754 | T | C | rs55755296 | A | T | rs11639425 | A | G |
| rs28701473 | A | G | rs2062062 | A | C | rs11639470 | C | G |
| rs1114600 | T | C | rs74007784 | T | C | rs11634776 | T | C |
| rs4924318 | A | C | rs8039171 | T | C | rs11629523 | A | G |
| rs4924319 | A | C | rs12439974 | A | G | rs11639489 | A | G |
| rs4244567 | A | G | rs12439975 | A | G | rs78104480 | A | T |
| rs12595209 | A | G | rs1038275 | T | C | rs4924345 | A | C |
| 15:39260109 | T | C | rs73401719 | A | G | rs4924346 | A | G |
| rs117155974 | A | C | rs74975389 | A | T | rs11638679 | T | C |
| rs75885297 | A | T | rs73401721 | A | C | rs6492882 | T | C |
| rs6492845 | T | C | rs10467905 | A | G | rs187522933 | A | G |
| rs7182885 | C | G | rs4327020 | T | C | rs8042633 | A | G |
| rs12899419 | A | C | rs7175606 | C | G | rs8042555 | A | C |
| rs6416431 | A | T | rs7176058 | A | G | rs4257183 | T | C |
| rs7167349 | T | C | rs1038276 | A | C | rs2868071 | A | G |
| rs3910263 | A | G | rs76699737 | T | G | rs4611426 | A | C |
| rs3910264 | T | C | rs76224009 | T | C | rs10520129 | A | G |
| rs3850775 | A | G | 15:39465776 | A | T | rs12708396 | A | G |
| rs7167621 | A | G | rs74007788 | T | C | rs11637272 | A | G |
| rs6492846 | A | G | rs28590036 | C | G | rs118045615 | T | C |
| rs3850776 | A | T | rs16968322 | C | G | rs4506869 | A | G |
| rs2033544 | A | C | rs1947908 | T | C | rs74869668 | T | C |
| rs12439381 | A | G | rs113901143 | T | C | rs35327539 | A | C |
| rs11632819 | C | G | rs72739348 | A | G | rs34893836 | A | G |
| rs149482976 | A | G | rs117530543 | A | G | rs1866832 | A | G |
| rs12439469 | A | G | rs8031934 | T | C | rs8023965 | T | G |
| rs1347267 | A | G | rs72739349 | T | C | rs8030000 | A | G |
| rs1347268 | C | G | rs72739350 | T | C | rs17622532 | A | G |
| rs1347269 | A | G | rs2673100 | A | G | rs62002336 | T | C |
| rs6492847 | T | G | rs2816589 | T | C | rs35391898 | A | G |
| rs6492848 | T | C | rs2200038 | C | G | rs10851388 | T | G |
| rs4924320 | T | C | rs60098272 | A | C | rs11634554 | C | G |
| rs12441917 | A | G | rs2816590 | T | G | rs28520337 | T | C |
| rs2411223 | T | C | rs2852083 | C | G | rs3910644 | T | C |
| rs2411224 | T | C | rs11635609 | A | G | rs3910645 | C | G |
| rs2411225 | A | G | rs2119408 | T | C | rs2053446 | T | C |
| rs2411226 | A | G | rs2816591 | A | G | rs11631459 | T | G |
| rs2164009 | T | C | rs2673099 | T | C | rs61561541 | T | C |
| rs2117205 | A | G | rs1371966 | A | G | rs8030047 | T | C |
| rs2117206 | T | C | rs11638941 | T | C | rs10048020 | T | C |
| rs11637063 | A | G | rs1440807 | A | G | rs7496544 | A | G |
| rs67559964 | A | G | rs2852081 | A | C | rs150751687 | T | C |
| rs11638272 | C | G | rs2852080 | T | C | rs72722991 | A | G |
| rs11638214 | A | C | rs16968353 | A | T | rs1440806 | T | C |
| rs4488414 | C | G | rs1371967 | A | G | rs17622678 | A | C |
| rs4640152 | C | G | rs117074140 | T | C | rs17622684 | C | G |
| rs58185015 | A | T | rs2119411 | A | C | rs1371962 | T | C |
| rs11855986 | C | G | rs1505273 | T | C | rs4545788 | A | G |
| rs10851374 | A | C | rs1505274 | T | C | rs28595601 | C | G |
| rs11070161 | C | G | rs17684145 | A | G | rs10152999 | A | C |
| rs11070162 | T | C | rs17684157 | A | G | rs56787586 | T | C |
| rs11635288 | C | G | rs2623476 | A | C | rs12050799 | A | G |
| rs7168790 | T | C | rs7170515 | A | G | rs9806403 | A | T |
| 15:39270627 | T | G | rs117446708 | T | C | rs9806435 | A | G |
| rs8028719 | T | C | rs2852078 | T | C | rs12594730 | A | C |
| rs8028720 | C | G | rs2673101 | A | G | rs73389474 | T | G |
| rs6492849 | T | C | rs156806 | A | G | rs59448536 | A | G |
| rs7168645 | T | C | rs1505278 | A | T | rs978740 | T | C |
| rs7169140 | A | G | rs2623474 | C | G | rs11633745 | T | C |
| rs7168969 | A | C | rs78238585 | A | G | rs11638501 | C | G |
| rs2411228 | A | G | rs72739354 | T | C | rs11638546 | A | G |
| rs2411229 | T | C | rs72739355 | A | G | rs11638505 | T | C |
| rs7174193 | A | G | rs2218209 | C | G | rs11638467 | A | C |
| rs12911831 | T | C | rs10520122 | T | C | rs55948686 | A | G |
| rs13380305 | A | C | rs16968383 | A | G | rs8024163 | T | C |
| rs13379944 | T | C | rs56244329 | T | C | rs1470147 | A | G |
| rs12708392 | T | C | rs76098304 | T | C | rs1470148 | A | G |
| rs2196116 | T | C | rs7170961 | T | C | rs58697049 | C | G |
| rs9919993 | T | G | rs150271229 | A | G | rs4923821 | T | G |
| rs9920253 | A | T | rs72739358 | T | C | rs12439394 | A | G |
| rs9920185 | A | C | rs7176210 | T | C | rs4573910 | C | G |
| rs72737117 | A | C | rs7175348 | A | G | rs1036718 | T | G |
| rs9920221 | A | G | rs4924340 | A | T | rs878728 | A | G |
| rs12591611 | T | C | rs74323846 | A | G | rs28570488 | A | C |
| rs8026182 | C | G | rs77018855 | T | G | rs4566132 | A | G |
| rs8026431 | A | C | rs16968400 | A | T | rs1837636 | T | C |
| rs8026436 | A | T | rs10520123 | A | C | 15:39661840 | A | T |
| rs72737120 | A | C | rs79994695 | T | C | rs4923822 | A | G |
| rs72737121 | A | T | rs76719016 | T | C | rs141616990 | A | T |
| rs8032264 | T | C | rs8028507 | T | C | rs4923823 | T | G |
| rs2411230 | A | G | rs16968410 | C | G | rs12902895 | A | C |
| rs2411231 | T | C | rs76157186 | A | T | rs77830251 | A | G |
| rs2411234 | T | G | rs56315914 | A | T | rs56926628 | A | G |
| rs892404 | A | T | rs74439363 | A | G | rs34680120 | A | C |
| rs892402 | A | G | rs62006546 | T | C | rs34137121 | C | G |
| rs117386953 | A | T | rs137917648 | A | C | rs56074533 | A | G |
| rs2411235 | T | C | rs28480630 | T | C | rs144634525 | T | C |
| rs719031 | A | G | rs17684646 | A | G | 15:39664956 | A | G |
| rs719032 | T | C | rs4924341 | A | G | rs4482254 | T | C |
| rs142986251 | A | G | rs79743941 | A | T | rs4614701 | C | G |
| rs964144 | A | G | rs16968421 | A | C | rs73393411 | T | C |
| rs2196115 | A | G | rs79602121 | A | G | rs73393413 | A | G |
| rs4924321 | T | C | rs75393310 | C | G | rs77223491 | T | C |
| rs75563926 | A | C | rs76099242 | A | T | rs77971909 | A | C |
| rs4923811 | A | G | rs72739361 | A | G | rs10851389 | A | G |
| rs7184047 | A | C | rs28630027 | C | G | rs28360709 | A | G |
| rs11070163 | A | G | rs17684749 | A | G | rs148506131 | T | C |
| rs11070164 | A | G | rs8037218 | T | G | rs28464045 | T | G |
| rs11070165 | C | G | rs16968439 | T | C | rs28540138 | T | C |
| rs12438600 | T | C | rs7183427 | T | G | rs28605540 | A | G |
| rs12441504 | C | G | rs12440685 | T | C | rs75287848 | T | G |
| 15:39277781 | T | C | rs10520125 | A | G | rs57181161 | A | G |
| rs11856921 | A | C | 15:39504194 | T | G | rs7175443 | T | C |
| rs12905966 | T | C | rs17684864 | T | G | rs60580147 | A | G |
| rs12905822 | A | G | 15:39504624 | T | C | rs12916089 | A | G |
| rs12905984 | T | C | rs75360543 | A | G | rs57434596 | A | G |
| 15:39278502 | T | C | rs9920660 | A | C | rs28887172 | A | G |
| rs12910426 | A | G | rs8040281 | T | C | rs28790585 | T | C |
| rs8034449 | A | C | rs73383239 | A | C | rs56358021 | A | G |
| rs116742941 | T | C | rs79257726 | T | C | rs35511994 | A | C |
| rs6492851 | A | G | 15:39507763 | T | C | rs67029160 | T | G |
| rs8040312 | A | G | rs16968450 | A | G | rs4924347 | T | C |
| rs4368145 | A | C | rs4923819 | A | G | rs117021520 | C | G |
| rs11070166 | T | G | rs11070175 | A | G | rs11070197 | T | C |
| rs4372662 | A | G | rs77656818 | A | G | rs12594280 | A | C |
| rs4290446 | T | C | rs16952107 | A | G | rs4312280 | A | G |
| 15:39281721 | A | G | rs16968451 | T | C | rs4441247 | A | T |
| rs12917381 | A | C | rs8030573 | T | C | rs4541049 | T | C |
| rs11855642 | T | C | rs117505383 | A | C | 15:39673762 | A | C |
| rs6492852 | T | C | rs276852 | A | C | rs149334770 | T | G |
| rs6492853 | T | C | rs62006548 | C | G | rs8039714 | A | C |
| rs7165634 | C | G | rs12440873 | A | G | rs79367167 | A | C |
| rs2082584 | C | G | rs7168182 | A | T | rs6492884 | A | G |
| rs2099394 | A | T | rs7169773 | A | T | rs8036564 | A | G |
| rs2099393 | A | G | rs7168534 | T | C | rs74008125 | T | G |
| rs7166250 | T | C | rs156777 | T | C | rs77294858 | T | C |
| rs2898671 | A | G | rs8031607 | T | C | rs77965329 | T | C |
| rs12910064 | A | T | rs7175771 | T | C | rs142622559 | T | C |
| rs11632632 | T | C | rs28885119 | A | G | rs60664658 | A | G |
| rs10851375 | T | C | rs156778 | A | G | rs7167226 | A | G |
| rs55925003 | A | G | rs56108070 | T | G | rs149363887 | T | C |
| rs1824492 | A | C | rs146333101 | A | G | rs11631466 | T | C |
| rs1824493 | A | T | rs7178098 | A | G | rs11634087 | A | C |
| rs1824494 | A | G | rs56165890 | A | C | rs7180659 | T | G |
| rs1841439 | A | C | rs2047943 | T | C | rs4924349 | A | T |
| rs149592852 | T | C | rs2047944 | T | C | rs75367938 | T | C |
| rs7161833 | A | C | rs78401978 | A | G | rs56214200 | A | G |
| rs1368807 | T | G | rs76037333 | A | C | rs7183019 | A | C |
| rs1368806 | T | G | rs11636346 | T | C | rs8043207 | A | G |
| rs1347270 | T | C | rs80014839 | A | C | rs8026954 | A | C |
| rs10851376 | T | C | rs77821708 | A | G | rs11636438 | T | C |
| rs10851377 | T | G | rs156795 | A | G | rs4597287 | A | G |
| rs79898844 | T | G | rs166723 | T | C | rs112953940 | T | C |
| rs2411236 | T | C | rs2673103 | A | G | rs72725006 | T | C |
| rs117332504 | A | G | rs6492864 | T | C | rs3973365 | A | G |
| rs1433891 | T | C | rs276863 | T | C | rs59068114 | A | G |
| s78911305 | A | G | rs8035407 | A | G | rs3973349 | A | C |
| rs2035061 | T | C | rs116895886 | T | G | rs3973348 | A | G |
| rs2035060 | T | C | rs8036004 | A | G | rs56032964 | A | G |
| rs2164010 | A | T | rs8036333 | A | G | rs4408513 | A | C |
| rs2019492 | T | C | rs1847662 | A | G | rs3960597 | A | G |
| rs1380937 | T | C | rs156796 | C | G | rs74008128 | A | T |
| rs67279504 | A | C | rs13329368 | A | G | rs55848083 | A | G |
| rs7180968 | C | G | rs156797 | C | G | rs11635229 | C | G |
| rs11070167 | A | C | rs156798 | C | G | rs7174276 | C | G |
| rs112138203 | T | C | rs156799 | A | G | rs12148895 | A | G |
| rs2164011 | A | G | rs75390446 | T | C | rs78045298 | A | G |
| rs2117207 | T | C | rs77232017 | A | G | rs73393456 | T | C |
| rs1433893 | T | G | rs59122632 | T | C | rs72725007 | T | C |
| rs1350748 | T | C | rs79055809 | T | C | rs11631290 | T | C |
| rs1433892 | T | C | rs117570016 | T | C | rs117637933 | T | G |
| rs116924548 | C | G | rs150598670 | T | G | rs59238363 | A | T |
| rs1368811 | C | G | rs275408 | C | G | rs139738435 | C | G |
| rs11630969 | T | C | 15:39526147 | T | C | rs77912390 | T | C |
| rs1991886 | T | G | rs156782 | C | G | rs56313937 | C | G |
| rs1865281 | C | G | rs6492865 | A | C | rs4522393 | T | C |
| rs2898672 | T | C | rs1394755 | T | C | rs77742801 | A | G |
| rs2126038 | A | G | rs1394754 | A | G | rs8038785 | T | C |
| rs2126037 | A | C | rs76238861 | C | G | rs11070198 | T | C |
| rs2411237 | A | G | rs1394753 | T | C | rs75126934 | A | T |
| rs7173881 | A | T | rs79158653 | A | C | rs11630143 | T | C |
| rs2196117 | T | C | rs8030447 | A | C | rs4306475 | T | C |
| rs117160421 | T | C | rs80239187 | T | C | rs11630296 | T | G |
| rs118055981 | C | G | rs76919724 | A | G | rs11635586 | A | T |
| rs12899025 | A | G | rs79025630 | T | C | rs4556763 | T | C |
| rs117943186 | T | C | rs1394752 | A | G | rs4924350 | A | G |
| rs11070168 | A | G | rs8037339 | T | C | rs4244569 | A | G |
| rs72737163 | T | C | rs8037733 | T | C | rs12906753 | A | C |
| rs1982101 | A | T | rs142226604 | C | G | rs12907239 | C | G |
| rs8029263 | A | G | rs7181501 | A | G | rs58280064 | A | T |
| rs12441780 | A | G | rs169345 | A | C | rs60313653 | T | G |
| rs12442496 | T | G | rs8043204 | T | C | rs77598024 | A | G |
| rs7180259 | A | C | rs2657211 | A | G | rs112148080 | A | G |
| rs995107 | T | C | rs276854 | A | G | rs73393472 | T | C |
| rs75109649 | A | G | rs78559446 | T | C | rs11855573 | A | G |
| rs72737170 | C | G | rs276855 | T | C | rs11634541 | A | G |
| rs61011574 | A | T | rs276856 | A | G | rs72725013 | T | C |
| rs1380938 | T | C | rs117637167 | C | G | rs4622487 | T | G |
| rs2898673 | A | C | rs276857 | A | G | rs8026812 | T | C |
| rs12439080 | C | G | rs16968518 | T | C | rs4405525 | T | C |
| rs6492854 | T | C | rs1394759 | A | G | rs74602662 | C | G |
| rs8032159 | A | G | rs8027821 | A | C | rs4344715 | T | C |
| rs8032320 | C | G | rs276858 | T | C | rs113721830 | C | G |
| rs12913015 | T | C | rs1020180 | T | C | rs4417529 | A | G |
| rs1460795 | A | G | rs10851381 | A | G | rs62002352 | T | C |
| rs1460796 | C | G | rs166722 | A | G | rs11634167 | A | C |
| rs146593808 | A | G | rs66523236 | T | G | rs4244570 | A | G |
| rs1460797 | T | C | rs17612917 | A | C | rs9921020 | A | C |
| rs4924322 | A | G | rs156658 | A | G | rs148890456 | A | G |
| rs4923812 | T | C | rs2816587 | T | C | rs148106383 | T | C |
| rs4923813 | T | G | rs28728196 | A | G | rs59431794 | T | G |
| rs4924323 | T | C | rs156794 | T | C | rs111659113 | A | G |
| rs4923814 | A | C | rs62006047 | T | C | | | |

TABLE 48
IPF risk variants associated with PUFIN3 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs11632437 | T | C | rs7177787 | A | G | rs111928892 | T | C |
| rs1055725 | T | C | rs10852031 | A | G | rs76754134 | A | G |
| rs7167179 | A | G | rs11853624 | A | G | rs72733149 | T | C |
| rs72733132 | A | G | rs11632791 | A | G | rs9920784 | A | G |
| rs4417528 | A | C | rs79840553 | A | G | rs76719447 | A | T |
| rs4429265 | T | C | rs75276658 | C | G | rs4924308 | T | G |
| rs4614698 | A | G | rs72733142 | A | C | rs76065080 | T | G |
| rs72733134 | T | C | rs34225173 | A | G | rs80185266 | A | T |
| rs72733135 | T | C | rs12102059 | A | C | rs6496013 | T | C |
| rs983743 | T | C | rs72733146 | T | C | rs6496014 | T | C |
| rs12916564 | A | C | rs17660474 | T | C | rs17660503 | A | G |
| rs77115844 | T | C | rs72733147 | T | C | rs7176726 | A | G |
| rs76850379 | C | G | rs72733148 | T | C | | | |

TABLE 49
IPF risk variants associated with PUFIN4 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs9294970 | T | C | rs146080332 | A | G | rs10945389 | T | C |
| rs9294971 | A | C | rs193281323 | A | G | rs73040109 | T | G |
| rs3763267 | T | C | rs6455467 | T | C | rs12183731 | T | C |
| rs3763266 | T | C | rs7769958 | T | G | rs9346625 | A | G |
| rs12212161 | T | C | rs1475138 | T | C | rs73040115 | A | G |
| rs12210740 | A | G | rs1475139 | T | C | rs58473311 | A | G |
| rs73038069 | A | G | rs34927895 | A | G | rs1322642 | C | G |
| rs12203887 | T | C | rs7750606 | A | G | rs9766794 | A | G |
| rs183037737 | A | G | rs9455893 | A | C | rs12661910 | T | C |
| rs117773632 | A | G | rs1331981 | T | C | rs9766355 | A | G |
| rs981010 | A | G | rs7752098 | T | C | rs183293345 | A | T |
| rs9689800 | A | T | rs7751131 | A | G | rs115579551 | C | G |
| rs9355135 | T | C | rs1331982 | T | C | rs9294973 | T | C |
| rs144699622 | T | G | rs1331983 | T | C | rs183637004 | T | C |
| rs73038073 | A | C | rs9455545 | A | C | rs9689249 | A | G |
| rs9688479 | A | G | rs73038102 | A | G | rs9688864 | A | G |
| rs9689304 | T | C | rs2104217 | T | C | rs9455897 | A | T |
| rs10945386 | C | G | rs9364359 | A | G | rs12180073 | A | G |
| rs73038079 | T | C | rs9364360 | A | G | rs9689350 | T | G |
| rs148108998 | T | C | rs9355136 | T | C | rs9505941 | A | T |
| rs4708600 | T | G | rs9364361 | T | C | rs9689153 | T | C |
| rs4708414 | C | G | rs9455896 | C | G | rs1322640 | T | C |
| rs73038086 | A | G | rs78509414 | T | C | rs1322639 | A | G |
| rs1888835 | T | C | rs140912195 | T | G | rs9689079 | A | G |
| rs11751421 | T | C | rs9346509 | A | G | rs9689481 | T | G |
| rs13204417 | A | G | rs61323225 | A | G | rs9689273 | T | G |
| rs55668560 | C | G | rs149618752 | T | C | rs911972 | A | C |
| rs12197486 | C | G | rs112270844 | A | G | rs9689193 | A | G |
| rs1475135 | T | C | rs2208831 | T | C | rs9455898 | C | G |
| rs1358978 | T | G | rs2208830 | T | C | rs10945391 | A | G |
| rs1475136 | C | G | rs9294972 | A | G | rs10945392 | T | C |
| rs1475137 | A | G | rs9364202 | T | G | | | |

TABLE 50
IPF risk variants associated with PUFIN5 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs17002201 | T | C | rs34106002 | T | C | rs344111 | A | T |
| rs34892362 | T | C | rs35309423 | A | T | rs35228159 | A | G |
| rs115455080 | T | G | rs34451825 | A | G | rs35780569 | T | C |
| rs13113476 | A | G | rs10518156 | T | C | rs34494372 | A | G |
| rs6848654 | A | C | rs56368232 | A | G | rs58700276 | A | C |
| rs6826310 | A | G | rs75461947 | T | C | rs56410305 | A | C |
| rs147818087 | C | G | rs4859461 | T | C | rs344110 | T | C |
| rs35142533 | T | C | rs116744869 | T | C | rs344109 | T | C |
| rs13127172 | A | G | rs62303168 | A | G | rs7657830 | A | G |
| rs17002205 | C | G | rs6815123 | T | C | rs7680361 | T | C |
| rs13129187 | T | C | rs114330729 | T | C | rs77821553 | C | G |
| rs71607375 | T | G | rs34992898 | A | G | rs7658629 | A | G |
| rs114596400 | A | G | rs11727930 | T | C | rs11736026 | T | C |
| rs113009524 | A | G | rs6532612 | A | G | rs74886079 | C | G |
| rs71607376 | T | C | rs7678615 | T | C | rs553311787 | T | G |
| rs13141990 | A | G | rs7661695 | A | G | rs716525 | T | C |
| rs13143734 | T | C | rs4859715 | A | G | rs55889196 | T | C |
| rs13130226 | T | C | rs114125115 | A | G | rs35530196 | T | C |
| rs35471029 | C | G | rs55650799 | T | G | 4:77717963 | A | C |
| rs7659925 | T | C | rs344115 | T | C | rs34462859 | T | C |
| rs7676973 | A | G | 4:77707008 | A | G | rs188040922 | T | G |
| rs7660736 | T | C | 4:77707209 | T | C | rs344108 | A | G |
| rs13112443 | T | C | rs17002208 | T | C | rs149502180 | T | C |
| 4:77692534 | T | C | rs11727699 | A | T | rs11097519 | A | G |
| rs34384644 | A | G | rs11727700 | A | T | rs344107 | T | C |
| rs35569366 | A | G | rs112962774 | T | C | rs17324961 | A | G |
| rs34973367 | C | G | rs72663250 | A | G | rs115755791 | C | G |
| rs36013312 | A | G | rs114263002 | T | C | rs12646332 | T | C |
| rs36057275 | T | G | rs34869090 | A | C | rs344102 | T | C |
| rs6848493 | C | G | rs35127460 | T | C | rs7669515 | A | G |
| rs6826927 | A | T | rs74562887 | A | T | rs76427371 | C | G |
| rs344096 | A | G | rs344113 | A | G | rs10035073 | T | C |
| rs6854121 | C | G | rs7699856 | A | G | rs79205213 | A | C |
| rs6831992 | A | G | rs344112 | T | C | rs9998681 | A | G |
| rs4859713 | T | C | rs57836169 | T | C | | | |

TABLE 51
IPF risk variants associated with PUFIN6 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs1800012 | A | C | rs16970089 | A | G | rs2247073 | T | C |
| rs9898186 | T | C | rs60517067 | C | G | rs2247074 | A | G |
| rs2269336 | C | G | rs138021013 | T | C | rs2103273 | T | C |
| rs1107946 | A | C | rs59137583 | C | G | rs1881142 | T | C |
| rs62062999 | A | G | rs62063000 | T | G | rs9909503 | T | G |
| rs113706230 | A | G | rs12325763 | C | G | rs61754902 | T | G |
| rs2586501 | T | C | rs11652362 | T | G | rs1406012 | T | G |
| rs75275039 | C | G | rs2246839 | A | G | rs112256951 | T | C |
| 17:48284268 | T | C | rs2586502 | A | C | rs2696278 | A | G |
| rs76917178 | A | G | rs16970094 | C | G | rs72826522 | T | C |
| rs78270168 | T | C | rs16970096 | T | C | | | |
| rs111286707 | T | C | rs1569117 | A | G | | | |

TABLE 52
IPF risk variants associated with PUFIN7 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs13025811 | T | C | rs1004071 | T | C | rs1011146 | T | C |
| rs3113503 | C | G | rs1004070 | T | G | rs112534738 | T | C |
| rs16854802 | A | G | rs3821350 | T | C | rs3732353 | T | C |
| rs1004072 | T | C | rs1011145 | T | C | | | |

TABLE 53
IPF risk variants associated with PUFIN8 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs61738025 | T | C | rs150408645 | T | C | rs7150659 | T | C |
| rs11622992 | T | C | rs2304707 | T | G | rs3784028 | T | C |
| rs75069872 | A | G | rs57176747 | A | G | rs11159089 | A | G |
| rs61980881 | A | T | rs61980885 | T | G | rs61654245 | T | C |
| rs61980882 | T | C | rs10142206 | A | G | rs3729502 | T | G |
| rs10136102 | A | G | rs55762670 | A | G | rs1008007 | A | G |
| rs55927952 | A | G | rs7148764 | T | C | rs28627652 | A | G |

TABLE 54
IPF risk variants associated with PUFIN9 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs1878199 | A | G | rs1040186 | A | T | rs2176267 | C | G |
| rs78706466 | T | C | rs17358566 | T | C | rs41265581 | A | C |
| rs6434304 | T | C | rs116329144 | A | G | rs1914037 | A | G |
| rs6434305 | T | C | rs13306264 | A | G | rs17292943 | A | G |
| rs62181723 | T | C | rs74729314 | A | G | rs41265557 | T | C |
| rs12693524 | A | G | rs113452130 | A | G | rs17358825 | A | T |
| rs150239545 | T | G | rs76955295 | A | C | rs2056156 | T | C |
| rs11675837 | A | G | rs1878201 | A | G | rs41265563 | A | G |
| rs116244378 | T | C | rs2351420 | A | C | rs16830973 | A | G |
| rs1878200 | A | T | rs1878202 | T | C | rs145536351 | T | C |
| rs16830961 | A | G | 2:189843516 | A | G | | | |
| rs2138533 | T | C | rs1516454 | A | G | | | |

TABLE 55
IPF risk variants associated with PUFIN10 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs74046265 | T | C | rs72636989 | A | G | rs35705950 | T | G |

TABLE 56
IPF risk variants associated with PUFIN11 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| rs55667133 | T | G | rs59036341 | A | T | rs146005148 | A | G |
| rs191224316 | A | G | rs139385675 | T | C | rs12207522 | A | G |
| rs56172843 | A | G | rs12179208 | A | G | rs145994398 | T | C |
| rs111887779 | A | G | rs10943222 | A | G | rs34193681 | T | C |
| rs2095664 | A | T | rs10943223 | T | C | rs137900026 | T | C |
| rs34179553 | T | C | rs10943224 | A | G | rs62414149 | T | C |
| rs186716231 | A | G | rs140361695 | T | C | rs35890626 | T | C |
| rs73451893 | T | G | rs35308030 | A | C | rs79088609 | A | C |
| rs962009 | A | G | rs35512580 | A | G | rs76598219 | A | G |
| rs962008 | A | C | rs35858009 | T | G | rs12204535 | A | C |
| rs112003867 | A | G | rs78387010 | T | C | rs145917917 | T | G |
| rs1002361 | A | T | rs4993833 | A | G | rs140342865 | A | G |
| rs117121230 | T | C | rs4993832 | T | C | rs117632924 | T | C |
| rs59398503 | T | C | rs73758176 | T | G | rs77727358 | T | C |
| rs76977545 | A | G | rs73758177 | A | G | rs117343483 | A | G |
| rs75043180 | A | C | rs73758178 | T | C | rs145902310 | A | G |
| rs146238727 | T | G | rs7774530 | A | C | rs118078688 | A | T |
| rs188571501 | C | G | rs7774670 | A | C | rs112217081 | T | C |
| rs6922268 | A | G | rs76556688 | T | C | rs116859015 | A | G |
| rs12526043 | T | C | rs7775022 | A | C | rs79761769 | A | G |
| rs72968406 | T | C | rs9360856 | T | C | rs76379040 | T | C |
| rs73758155 | T | C | rs7775735 | A | G | rs10455306 | T | C |
| rs35779502 | T | G | rs9360857 | T | C | rs150016949 | C | G |
| rs75587050 | A | G | rs7761525 | T | C | rs149021062 | T | C |
| rs4084884 | A | G | 6:75549552 | T | C | rs35264814 | T | C |
| rs4084883 | A | G | rs2353329 | A | G | rs4706591 | T | C |
| rs61421532 | A | G | rs112654522 | T | C | rs6918848 | A | G |
| rs57530644 | T | C | rs2353330 | T | G | rs78874927 | T | C |
| rs73454012 | A | G | rs4645384 | T | G | rs117875698 | T | C |
| rs5020129 | C | G | rs4565264 | A | G | rs75444912 | A | C |
| rs74724774 | T | C | rs7756419 | A | C | rs112394072 | A | G |
| 6:75520837 | C | G | rs7756547 | T | C | rs34850405 | T | C |
| rs147013219 | T | C | rs7756570 | C | G | rs78524645 | T | C |
| rs7744026 | A | T | rs7756722 | A | G | rs75880919 | C | G |
| rs72968418 | T | C | rs9350576 | A | G | rs10943230 | T | G |
| rs72968419 | T | C | rs111288519 | T | C | rs113591482 | A | G |
| rs9443065 | A | G | rs9350577 | A | G | rs144543645 | C | G |
| rs118164490 | A | G | rs9341500 | A | T | rs10455307 | A | G |
| rs73454033 | T | C | rs9360858 | T | C | rs113974127 | A | C |
| rs73454038 | A | C | rs9343240 | T | C | rs4298307 | T | G |
| rs73454042 | T | G | rs9343241 | A | C | rs138715417 | T | G |
| rs73454045 | A | G | rs9343242 | T | C | rs62414157 | A | T |
| rs111559597 | T | G | rs62438811 | T | C | rs112398977 | A | G |
| rs4708167 | A | G | rs9343243 | T | C | rs4475293 | A | C |
| rs7768703 | A | G | rs9343244 | A | C | rs116874872 | A | G |
| rs58205256 | A | G | rs9343245 | T | C | rs62414159 | A | G |
| rs4084882 | A | G | rs9343246 | A | G | rs138482046 | A | G |
| rs7756101 | T | C | rs72968446 | T | C | rs553591134 | A | G |
| rs7760080 | T | C | rs4400180 | A | C | rs149808704 | T | C |
| rs7760092 | T | C | rs75373407 | C | G | rs76645693 | A | T |
| rs73454061 | T | C | rs4339417 | A | G | rs4346826 | A | G |
| rs73454063 | T | G | rs4112831 | A | G | rs144191157 | T | C |
| rs73454065 | A | G | rs6938007 | T | C | rs117452604 | A | G |
| rs12524330 | A | G | rs2353331 | A | G | rs117663742 | T | C |
| rs12527799 | T | G | rs4312940 | T | C | rs140001013 | A | C |
| rs2152827 | T | C | rs9443079 | T | C | rs9885908 | A | T |
| rs2152826 | A | T | rs9359111 | A | G | rs180820615 | T | C |
| rs7751557 | A | C | rs9341502 | T | C | rs4518462 | A | G |
| rs7756234 | A | G | rs9341503 | T | C | rs62415663 | A | G |
| rs72968425 | A | G | rs76557485 | T | C | rs78123791 | A | G |
| rs7756312 | A | C | rs2353332 | A | T | rs13209662 | C | G |
| rs4605845 | A | G | rs2353333 | T | G | rs12524721 | A | T |
| rs4279387 | A | G | rs12192355 | A | C | rs62415664 | T | C |
| rs4569926 | A | G | rs2184927 | C | G | rs4291062 | A | G |
| rs4084881 | A | G | rs4112830 | T | C | 6:75739925 | A | G |
| rs113413053 | A | G | rs34153603 | A | T | rs17185571 | A | C |
| rs73454084 | A | C | rs9447312 | A | G | rs12212942 | T | G |
| rs73454085 | A | G | rs9352167 | T | C | rs78241113 | T | C |
| rs73454086 | T | C | rs9447313 | T | G | rs12530065 | T | C |
| rs73454087 | A | G | rs9443082 | T | C | rs9360872 | A | G |
| rs73454091 | T | G | rs9447314 | A | G | rs4432952 | A | G |
| rs73454093 | T | G | rs72968450 | A | G | rs138573823 | T | C |
| rs73454094 | A | C | rs9293982 | A | G | rs145793243 | A | G |
| rs12524618 | A | G | rs191107167 | T | C | rs10455308 | T | C |
| rs12529890 | T | C | rs77921955 | T | C | rs10455309 | T | C |
| rs60869385 | T | C | rs4112828 | T | C | rs11758990 | A | C |
| rs9343235 | A | G | rs6932300 | A | G | rs75026337 | A | G |
| rs9360853 | T | C | rs6912253 | T | C | rs6453808 | C | G |
| rs58085300 | A | G | rs55655559 | C | G | rs6453809 | T | C |
| rs7755255 | A | G | rs55820941 | A | G | rs73462024 | T | C |
| rs188113657 | A | G | rs72968451 | T | C | rs11754104 | C | G |
| rs6918679 | T | C | rs77989827 | T | C | rs4495247 | T | C |
| 6:75532966 | T | C | rs6453788 | C | G | rs4569931 | A | G |
| rs61556471 | A | T | rs7749251 | T | G | rs6914147 | T | C |
| rs60280950 | T | C | rs9360862 | A | G | rs4413578 | T | C |
| rs4083898 | T | C | rs10081106 | T | G | rs148055133 | T | C |
| rs4083897 | T | C | rs9293983 | A | G | rs9359116 | A | C |
| rs1890091 | A | G | rs9293984 | A | G | rs112751227 | T | C |
| rs1890090 | T | C | rs57728927 | A | C | rs112023529 | A | G |
| rs73456110 | A | T | rs4708168 | T | C | rs4571524 | A | T |
| rs13212125 | A | G | rs61313163 | T | G | rs4560603 | T | G |
| rs35859490 | T | G | rs116982671 | A | T | rs11758551 | A | T |
| rs6909981 | T | C | rs147173490 | A | G | rs4708171 | A | G |
| rs112467026 | C | G | rs4320321 | C | G | rs56310973 | T | C |
| rs111611608 | A | C | rs945819 | T | C | rs4579316 | T | C |
| rs6910698 | T | C | rs76073069 | T | C | rs80161316 | T | C |
| rs73456114 | A | G | rs113853886 | A | G | rs140231712 | T | C |
| rs16885685 | A | G | rs9447323 | A | G | rs4601093 | T | C |
| rs73456118 | C | G | rs78635740 | A | C | rs4509089 | T | G |
| rs79231534 | A | C | rs117120077 | C | G | 6:75762044 | A | G |
| rs117147262 | A | G | rs75775098 | A | C | rs74384277 | T | C |
| rs111872955 | T | C | rs117549198 | C | G | rs6919710 | C | G |
| rs9352163 | C | G | rs80072717 | T | G | rs117453878 | A | G |
| rs9352164 | A | G | rs9352168 | A | T | rs73462037 | C | G |
| rs16879413 | T | G | rs12212000 | A | G | rs9360875 | T | G |
| rs16879415 | A | C | rs80255441 | T | C | rs79433649 | T | C |
| rs17786760 | A | G | 6:75577861 | T | C | rs1576754 | A | G |
| rs16879417 | A | G | rs150319951 | A | G | rs7773524 | A | C |
| rs12177973 | T | G | rs6903537 | A | G | rs9352187 | T | C |
| rs9447290 | A | C | rs188155694 | T | C | rs11965039 | T | C |
| rs9443067 | T | G | rs9443093 | A | G | rs28838319 | T | C |
| rs148925739 | A | G | rs16885806 | T | C | rs1304580 | A | G |
| rs118182668 | A | G | rs117826244 | T | C | rs7751171 | T | C |
| rs9447294 | T | C | rs117905363 | A | G | rs2065421 | T | G |
| rs7749761 | A | G | rs78028211 | T | C | rs118043261 | A | G |
| rs113148354 | T | G | rs79074021 | A | G | rs602160 | A | C |
| rs16820 | A | G | rs140267182 | T | C | rs9343264 | T | G |
| rs79187151 | T | C | rs78496860 | A | G | rs148279530 | A | G |
| rs9343237 | A | G | rs143056318 | A | G | rs117693620 | T | C |
| rs9443068 | A | C | rs62438843 | T | G | rs726242 | A | G |
| rs9443069 | A | T | rs12197372 | A | G | rs146202057 | T | G |
| rs9443070 | A | G | rs12173437 | A | G | rs7771969 | A | G |
| rs9447296 | T | G | rs7743423 | T | C | rs62415666 | T | C |
| rs9443072 | A | G | rs117982514 | A | G | rs6453812 | A | G |
| rs9447297 | T | C | rs117624271 | T | G | rs9360878 | T | C |
| rs12526503 | A | G | rs9343248 | T | G | rs6928356 | A | G |
| rs9443073 | A | G | rs7742016 | A | G | rs138334820 | A | G |
| rs7774441 | A | G | rs7772613 | T | C | rs1412275 | T | C |
| rs9447300 | A | G | rs9447347 | T | G | rs117770271 | C | G |
| rs72968442 | A | G | rs140740500 | A | G | rs9343266 | A | T |
| rs73758167 | A | C | 6:75611839 | T | C | rs148181992 | A | G |
| rs73758168 | A | T | rs186953283 | T | C | rs117260333 | T | G |
| rs59237305 | T | C | rs140826576 | A | G | rs765796 | A | G |
| rs60018538 | A | G | rs7757667 | A | G | rs77856908 | T | C |
| rs78747279 | T | C | rs150427930 | A | T | rs73745751 | A | C |
| rs9447302 | A | G | rs146358472 | T | G | rs6916346 | T | C |
| rs4395691 | A | G | rs149107361 | C | G | rs75496051 | T | C |
| rs4084878 | A | G | rs62438845 | A | C | rs554152 | T | C |
| rs4397194 | A | G | rs35323107 | T | C | rs885793 | A | G |
| rs4276467 | T | C | rs111449290 | T | C | rs79879478 | T | C |
| rs4285291 | T | C | rs75779365 | A | G | rs62415668 | A | G |
| rs6909748 | A | G | rs116948181 | A | G | rs116865777 | T | C |
| rs147279468 | T | C | rs193179432 | C | G | rs970547 | T | C |
| rs7748489 | A | C | rs76762868 | A | G | rs2273264 | A | G |
| rs7752612 | A | G | rs141605327 | T | C | rs150035394 | A | C |
| rs61128758 | T | C | rs13193857 | T | C | rs3818138 | T | C |
| rs7752504 | T | C | rs35363184 | A | G | rs76979570 | A | G |
| rs7772922 | T | C | rs192758048 | C | G | rs516533 | A | G |
| rs7752681 | A | G | rs2184925 | T | G | rs3765496 | T | C |
| rs79528332 | T | C | rs56138234 | A | T | rs10755329 | A | T |
| rs7753687 | A | C | rs141343508 | A | G | rs9447442 | T | C |
| rs9360855 | T | C | rs2485448 | T | C | rs9443154 | T | C |
| rs9343238 | A | G | rs74354527 | A | G | rs10943236 | A | G |
| rs9352165 | A | G | rs118040907 | T | C | | | |
| rs58720983 | T | C | rs11751258 | T | C | | | |

TABLE 57
IPF risk variants associated with PUFIN12 transcript.

| | Non- | | | Non- | | | Non- | |
|---|---|---|---|---|---|---|---|---|
| | effect | Effect | | effect | Effect | | effect | Effect |
| rsid | Allele | Allele | rsid | Allele | Allele | rsid | Allele | Allele |
| 16:86879475 | T | C | rs12921019 | T | C | rs8055677 | A | G |
| rs9674258 | A | T | rs74655565 | A | T | rs12927839 | A | C |
| rs4369671 | A | G | rs12919946 | A | G | rs8055704 | A | G |
| rs117009827 | T | C | rs79529294 | A | T | rs12928693 | A | G |
| rs12921251 | A | G | rs34704948 | A | G | rs149585321 | A | G |
| rs28494023 | T | C | rs71390869 | T | C | rs7202217 | T | C |
| rs74323672 | A | C | rs78747828 | A | G | rs7184988 | T | C |
| rs28393836 | T | C | rs74662937 | T | C | rs72812366 | A | G |
| rs7498805 | A | G | rs13332828 | A | G | rs12448736 | T | C |
| rs61137455 | C | G | rs4843482 | A | G | rs117489395 | C | G |
| rs7195233 | A | G | rs4843483 | A | G | rs117110764 | A | C |
| rs7195420 | A | G | rs56109913 | A | G | rs74567577 | T | G |
| rs4843467 | T | C | rs74663663 | A | G | rs9932157 | T | C |
| rs60416911 | A | C | rs6539986 | T | C | rs9939889 | C | G |
| rs113681680 | A | G | rs76384593 | A | T | rs56200259 | T | C |
| rs78638559 | A | G | rs12598069 | T | G | rs112419638 | C | G |
| rs4076536 | A | G | rs80283055 | A | G | rs4843504 | T | C |
| rs7186442 | T | C | rs183958843 | A | G | rs112613632 | T | C |
| rs9932284 | T | C | rs60921554 | A | G | rs12920049 | T | C |
| rs4843468 | C | G | rs60076424 | A | G | rs6539990 | C | G |
| rs80273810 | A | C | rs11864370 | T | C | rs8049677 | A | G |
| rs11642396 | A | G | rs76857713 | A | C | rs72812370 | A | T |
| rs11117220 | T | C | rs59585579 | T | C | rs72812371 | T | C |
| 16:86885593 | A | T | rs73258983 | A | T | rs143269780 | A | T |
| rs56890992 | T | G | rs12447754 | A | G | rs7192767 | T | C |
| rs79255665 | A | G | rs7499881 | T | C | rs9932544 | T | C |
| rs149577182 | T | C | rs144565240 | A | G | rs9922741 | A | T |
| rs113607549 | T | C | rs7203386 | T | C | rs11641441 | T | G |
| rs113692961 | T | C | rs7204112 | A | G | rs11117238 | A | G |
| rs59899563 | T | C | rs4586440 | A | T | rs10863175 | T | C |
| rs77968447 | A | T | rs11859321 | T | C | rs10863176 | T | C |
| rs7188686 | T | C | rs55990773 | A | T | rs62043391 | T | C |
| rs7187708 | A | G | rs8046283 | C | G | rs4843212 | T | G |
| rs58059090 | T | C | rs118081598 | T | G | rs78822709 | C | G |
| rs117299787 | A | G | rs8045410 | A | G | rs9932385 | A | G |
| rs72808475 | A | G | rs4843484 | A | G | rs116890779 | A | T |
| rs181816535 | T | C | rs117551230 | A | T | rs4338811 | T | C |
| rs12929574 | T | G | rs75436742 | A | G | rs13335455 | A | G |
| rs4076987 | T | C | rs8047566 | C | G | rs112392227 | C | G |
| rs4076988 | T | C | rs12920171 | C | G | rs9935976 | T | C |
| rs12448444 | A | G | rs58328141 | A | G | rs8058785 | A | T |
| rs72808476 | T | C | rs73258997 | A | C | rs8058192 | T | C |
| rs55737755 | T | C | rs13337137 | T | C | rs8063400 | T | C |
| rs76315510 | C | G | rs77107138 | A | C | rs78665568 | T | C |
| rs11117223 | C | G | rs57046201 | T | C | rs147098394 | C | G |
| rs11117224 | T | C | rs4240783 | T | C | rs9931220 | T | C |
| rs11640888 | T | C | rs76436442 | T | C | rs9939098 | T | C |
| rs147375017 | T | C | rs11645930 | T | C | rs66765067 | C | G |
| rs73272939 | A | G | rs4843485 | A | T | rs72812376 | T | C |
| rs77349469 | C | G | rs118034203 | T | C | rs62043393 | T | G |
| rs72808479 | T | C | rs73260610 | A | G | rs9931925 | A | G |
| rs4843469 | C | G | rs75546753 | T | C | rs111618284 | T | C |
| rs62042460 | C | G | rs7197567 | T | C | rs72812379 | A | G |
| rs62042461 | A | G | rs78887866 | T | C | rs151226268 | A | T |
| rs11640239 | C | G | rs74315602 | C | G | rs72812380 | A | T |
| rs60489741 | T | C | rs4843486 | A | C | rs8048512 | A | G |
| rs11117225 | A | G | rs58569666 | T | C | rs72812382 | T | C |
| rs113520903 | A | T | rs72810315 | T | C | rs79560096 | A | C |
| rs80315349 | A | G | rs73260617 | A | G | rs55695281 | C | G |
| rs4843470 | T | G | rs4843489 | T | C | rs112615707 | T | C |
| rs4843471 | T | C | rs7205063 | T | C | rs12923543 | A | C |
| rs12926612 | C | G | rs7205083 | A | C | rs34845927 | T | G |
| rs73272960 | A | G | rs7205247 | T | C | rs72812385 | C | G |
| rs59643091 | T | G | rs7205276 | T | C | rs75142546 | A | G |
| rs60615201 | T | G | rs78546537 | A | G | rs62043394 | A | G |
| rs59200236 | T | C | rs34466951 | C | G | rs117556055 | A | G |
| rs113909467 | T | C | rs12921571 | A | C | rs12927908 | T | G |
| rs34204151 | A | T | rs12920112 | A | G | rs192353983 | A | G |
| rs7206774 | C | G | rs12921890 | A | C | rs62043395 | T | C |
| rs56048800 | T | C | rs80289825 | T | C | rs62043396 | C | G |
| rs75470476 | A | G | rs7404179 | A | G | rs13333744 | A | G |
| rs4843472 | C | G | rs62042508 | A | G | rs77399143 | T | C |
| rs4843473 | T | C | rs35788096 | A | T | rs55929045 | T | C |
| rs57634362 | A | G | rs76262852 | T | C | rs12934217 | T | C |
| rs12924082 | A | G | rs78520547 | A | C | rs117620285 | A | G |
| rs60937234 | A | G | rs185759813 | A | G | rs35474936 | T | C |
| rs12929168 | A | C | rs62042509 | C | G | rs62043397 | A | G |
| rs12933143 | T | C | rs73260633 | A | G | rs80184641 | T | G |
| rs140744663 | T | C | rs74589715 | T | C | rs3849238 | A | C |
| rs74966730 | T | G | rs11863071 | T | C | rs36036243 | T | C |
| rs74319428 | A | G | rs75886763 | C | G | rs73242494 | A | G |
| rs57715595 | T | C | rs56286510 | A | T | rs12447368 | A | G |
| rs35483955 | A | C | rs547193654 | A | C | rs36102483 | C | G |
| rs4843474 | A | G | rs571916143 | A | T | rs3849237 | T | C |
| rs4843475 | T | G | rs80057018 | T | C | rs12445522 | T | C |
| rs4843476 | A | G | 16:86984787 | C | G | rs35979179 | T | C |
| rs35565712 | A | G | 16:86985329 | T | C | rs3849236 | A | G |
| rs9933616 | A | G | rs75092194 | T | C | rs12934686 | T | G |
| rs9935865 | T | G | rs68173841 | T | C | rs12934703 | T | G |
| rs4843477 | T | C | rs12711468 | A | G | rs12711469 | T | C |
| rs13380717 | A | G | rs74037303 | C | G | rs2313582 | T | C |
| rs142442552 | A | C | rs13380436 | T | G | rs56102488 | A | G |
| rs75245445 | A | C | rs149583170 | A | T | rs72812390 | T | C |
| rs113407053 | T | C | rs11641052 | T | C | rs12926896 | T | C |
| rs111661051 | A | C | rs34389271 | A | G | rs7195670 | C | G |
| rs116913104 | A | G | rs11644894 | A | C | rs72812391 | T | G |
| rs112717578 | C | G | rs34563066 | T | C | rs72812392 | T | G |
| rs8050596 | A | G | rs11863581 | T | C | rs58217848 | A | G |
| rs3935244 | T | C | rs4843197 | C | G | rs12444869 | A | G |
| rs8053747 | C | G | rs74037304 | T | C | rs12445035 | A | C |
| rs57790970 | A | G | rs4550457 | A | G | rs12445562 | A | G |
| rs58797760 | C | G | rs34871649 | A | G | rs7201815 | A | C |
| rs7195867 | A | G | rs58422188 | T | G | rs117760950 | A | G |
| rs75279030 | A | G | rs12925082 | A | G | rs12050917 | A | T |
| rs77945601 | A | C | rs34093478 | C | G | rs12050920 | T | C |
| rs11117226 | T | C | rs62042513 | T | C | rs56169293 | T | C |
| rs34123501 | A | G | rs7404835 | T | G | rs12051142 | T | G |
| rs34528289 | T | C | rs72810326 | A | G | rs12051143 | A | G |
| rs112204969 | T | C | rs72810327 | A | G | rs12051162 | A | C |
| rs58763922 | A | G | rs11647668 | A | G | rs35542687 | C | G |
| rs113802822 | A | G | rs7200534 | A | G | rs35021400 | A | C |
| rs62042462 | A | G | rs67815507 | A | G | rs71390870 | T | C |
| rs4545822 | A | G | rs6539988 | A | C | rs4552032 | T | C |
| rs78949425 | A | G | rs143893149 | A | G | rs4525494 | T | C |
| rs34805040 | T | G | rs34030213 | C | G | rs111362724 | A | G |
| rs9889091 | A | G | rs62042514 | T | G | 16:87047545 | T | G |
| rs9937860 | T | G | rs72810331 | T | C | rs35422950 | A | C |
| rs12325063 | T | C | rs11649124 | A | G | rs79331727 | A | T |
| rs9925222 | T | C | rs55677614 | T | C | rs28464130 | A | C |
| rs4240780 | T | C | rs11649216 | A | G | rs8051603 | A | C |
| rs12325533 | A | G | rs11649573 | T | C | rs12447918 | T | G |
| rs55877164 | T | C | rs4843490 | T | C | rs8054887 | A | G |
| rs11642990 | T | C | 16:86991422 | T | C | rs8056525 | T | C |
| rs7198809 | A | G | rs11647106 | A | G | rs16942474 | C | G |
| rs4843189 | T | C | rs9796837 | T | C | rs13337926 | A | C |
| rs7201351 | A | C | rs4843491 | A | G | rs13337958 | T | C |
| 16:86911311 | A | T | rs4240784 | A | G | rs12448109 | A | G |
| 16:86911702 | T | C | rs4264397 | A | G | rs12929657 | A | C |
| rs55997116 | T | C | rs4467084 | T | C | rs192778571 | A | G |
| rs11640938 | A | G | rs9939522 | A | G | rs35303213 | A | G |
| rs182475626 | A | C | rs9921492 | T | C | rs7187757 | T | C |
| rs187999534 | A | G | rs7202909 | T | C | rs189088033 | A | G |
| rs12149188 | A | G | rs7186038 | A | T | rs112387031 | A | C |
| rs144552243 | T | G | rs57538453 | C | G | rs7205573 | C | G |
| rs4581707 | T | G | rs4371163 | A | G | rs12149906 | A | G |
| rs4480814 | T | C | rs72810340 | A | C | rs12149939 | A | C |
| rs4573932 | C | G | rs72810341 | T | C | rs16942482 | T | C |
| rs4399543 | A | G | rs76666726 | C | G | rs16942488 | T | C |
| rs4479233 | T | C | rs72810342 | C | G | rs16942492 | C | G |
| rs4335778 | A | G | rs72810343 | T | G | rs13331665 | A | G |
| rs12928641 | T | C | rs145287139 | T | C | rs11648116 | T | G |
| rs66792073 | T | C | rs4390601 | T | C | rs4065981 | C | G |
| rs7186655 | T | C | rs73260664 | A | G | rs57957159 | A | G |
| rs7202673 | A | G | rs117093779 | A | G | rs60603451 | T | G |
| rs11117227 | T | C | rs11646583 | T | G | rs8059092 | A | C |
| rs112281320 | A | C | rs9922063 | A | G | rs1109879 | T | C |
| rs55646952 | A | C | rs9922158 | A | G | rs8059461 | C | G |
| rs11643826 | A | G | rs151065592 | A | G | rs6539993 | C | G |
| rs78426657 | A | G | rs4843198 | A | G | rs12932192 | A | T |
| rs4843478 | T | C | rs76943123 | T | C | rs11862201 | T | C |
| rs117414633 | A | G | rs9935789 | A | G | rs1974861 | C | G |
| rs35640584 | T | C | rs9937993 | T | G | rs12103297 | A | G |
| rs4843190 | A | T | rs12599689 | T | C | rs1559337 | T | C |
| rs4843191 | C | G | rs55764104 | A | G | 16:87053713 | A | G |
| rs4843192 | T | C | rs56335607 | A | G | 16:87053729 | T | G |
| rs11645491 | C | G | rs117192810 | A | G | rs72812400 | T | C |
| rs4843479 | T | C | rs62042517 | T | C | rs929860 | T | G |
| rs4843480 | A | G | rs4843199 | C | G | rs929859 | A | C |
| rs143667308 | T | C | rs192095736 | A | C | rs929856 | C | G |
| rs12447695 | T | C | rs35000812 | C | G | rs17290774 | A | C |
| rs12051139 | T | C | rs4077868 | T | C | rs79460491 | C | G |
| rs59876150 | C | G | rs13335726 | A | G | rs17218537 | A | C |
| rs58607576 | C | G | rs13332352 | T | C | rs12929461 | A | T |
| rs56225926 | C | G | rs11646035 | T | C | rs17218565 | T | C |
| rs112088240 | C | G | rs4843492 | T | C | rs13336920 | A | G |
| rs7501319 | T | C | rs7196400 | A | G | rs17290872 | A | T |
| rs77698327 | T | C | rs7195882 | T | C | rs9674246 | T | C |
| rs4843193 | T | C | rs11643420 | T | C | rs60913481 | A | G |
| rs59346730 | A | G | rs7404555 | A | G | rs9673420 | A | G |
| rs77394698 | A | C | rs4843201 | C | G | rs77890800 | A | G |
| rs60650113 | C | G | rs9926799 | A | C | 16:87056584 | A | G |
| rs60518159 | A | G | rs9936705 | T | C | 16:87056588 | T | G |
| rs59870172 | T | C | rs56385411 | C | G | rs56198238 | T | C |
| rs73254791 | C | G | rs11117235 | C | G | rs4843507 | T | C |
| rs60114760 | A | C | rs34664502 | A | T | rs72812402 | C | G |
| rs116788197 | T | G | 16:87001662 | A | G | rs117437365 | T | C |
| rs59467960 | T | C | rs79589654 | A | G | rs116950924 | A | G |
| rs57991993 | T | C | rs62042519 | A | G | rs9673656 | T | C |
| rs4843194 | T | C | rs72810353 | A | G | rs12445782 | T | C |
| rs77838875 | A | T | rs34249329 | T | G | rs7202450 | T | G |
| rs75901144 | T | C | rs72810354 | A | T | rs11643698 | A | T |
| rs75663580 | T | C | rs78534188 | A | T | rs9806881 | T | G |
| rs75057596 | A | G | rs9935171 | T | C | rs9806862 | A | G |
| rs55901444 | T | C | rs73260695 | C | G | rs8054722 | A | G |
| rs57027199 | T | C | rs72810355 | A | G | rs4843216 | A | T |
| rs117899768 | A | G | rs72810357 | A | G | rs4843511 | T | C |
| rs55739482 | T | C | rs7187045 | A | G | rs36044788 | A | C |
| rs55803544 | A | G | rs72810358 | T | C | rs8061668 | A | G |
| rs76047588 | T | G | rs7188798 | C | G | rs9932122 | T | C |
| rs11641193 | T | C | rs74703824 | T | C | 16:87060197 | A | G |
| rs4372698 | A | G | rs5024159 | T | C | rs78460606 | T | C |
| rs59019175 | C | G | rs5024158 | A | G | rs58510517 | C | G |
| rs112855655 | T | C | rs139638064 | A | G | rs12932746 | A | G |
| rs76825291 | T | G | rs76701327 | C | G | rs17291200 | T | C |
| rs4305026 | T | C | rs111210062 | A | C | rs12595980 | T | G |
| rs9926421 | A | G | 16:87006267 | A | G | rs59880838 | A | G |
| rs60519549 | A | C | rs12930964 | T | C | rs6539994 | C | G |
| rs9926602 | A | G | rs554184041 | A | G | rs7198470 | T | C |
| rs75918370 | T | C | rs12926793 | T | C | rs16942558 | T | C |
| rs9932360 | C | G | rs12149690 | T | C | rs71390875 | T | C |
| rs76063539 | A | G | rs4843202 | A | G | rs13331527 | A | G |
| rs9932721 | T | C | rs12149435 | T | C | rs17291430 | A | G |
| rs4444348 | A | G | rs75735876 | T | C | rs75428640 | A | G |
| rs4451955 | A | G | rs4843493 | C | G | rs12711470 | A | C |
| rs4268754 | T | C | rs7201567 | A | G | rs72628297 | C | G |
| rs4589560 | T | C | rs34327282 | C | G | rs13332710 | A | G |
| rs9935451 | T | C | rs4843494 | A | C | rs148367517 | T | C |
| rs9935749 | T | C | rs7206742 | T | C | rs11642905 | C | G |
| rs61268128 | C | G | rs73260698 | A | T | rs78700173 | C | G |
| rs76497033 | T | C | rs12598138 | T | C | rs8063008 | T | C |
| rs56158691 | T | G | rs62042549 | A | C | rs13332913 | A | G |
| rs76866247 | T | C | rs111802031 | T | C | rs12449151 | T | C |
| rs77154859 | T | C | rs12448659 | C | G | rs72813912 | A | G |
| rs78362076 | T | C | rs12448660 | A | C | rs72813913 | A | G |
| rs11647070 | T | C | rs78123748 | T | C | rs12445424 | A | G |
| rs78089027 | A | G | rs13339459 | T | G | rs17769763 | A | G |
| rs77544811 | C | G | rs58716793 | T | C | rs35635713 | A | G |
| rs77789822 | A | G | rs8045549 | A | G | rs149287763 | A | C |
| rs77118693 | A | C | rs9921719 | T | C | rs7205934 | T | C |
| rs75519305 | T | C | rs9934291 | A | G | rs11859917 | T | C |
| rs71390865 | A | G | rs4843203 | T | C | rs144650513 | A | G |
| rs11117228 | T | C | rs72810364 | A | G | rs55957868 | A | T |
| rs11117229 | A | T | rs72810366 | T | C | rs28599585 | T | C |
| rs79553371 | T | C | rs77856883 | C | G | rs7202117 | A | G |
| rs11641964 | T | G | rs8051419 | A | G | rs13334832 | T | C |
| rs4405560 | T | C | rs73262508 | C | G | rs75848718 | A | C |
| rs4617867 | C | G | rs57789292 | A | G | rs72628298 | T | C |
| rs117703016 | T | C | rs7186884 | T | G | rs2161646 | A | G |
| rs4441282 | A | T | rs62042550 | T | C | rs7201182 | A | G |
| rs4372697 | T | C | rs75509731 | A | C | rs58224561 | C | G |
| rs4473194 | A | C | rs10863173 | T | G | rs57132249 | A | G |
| rs4344755 | A | C | rs148559094 | T | C | rs11646105 | T | C |
| rs12445761 | T | C | rs11641463 | T | C | rs12595901 | A | G |
| rs12445762 | T | C | rs72810370 | T | C | rs72813919 | A | G |
| rs3934493 | A | G | rs8047569 | A | G | rs7191560 | T | C |
| rs3934494 | T | G | rs4843496 | A | G | rs4843514 | C | G |
| rs76322507 | A | C | rs113566637 | A | G | rs72813923 | C | G |
| rs73257016 | C | G | rs4843497 | A | C | rs17769781 | C | G |
| rs79680006 | A | G | rs4843498 | T | C | rs72813924 | A | G |
| rs72810304 | A | C | rs12925828 | T | G | rs78703012 | A | G |
| rs4075601 | T | C | rs73262512 | T | C | rs3860272 | A | G |
| rs4075602 | A | G | rs6539989 | C | G | rs78826894 | A | G |
| rs7186606 | A | G | rs62042551 | A | T | rs77405367 | C | G |
| rs4075600 | A | G | rs73262514 | C | G | rs17769799 | T | G |
| rs4076155 | T | G | rs62042552 | T | G | rs72813925 | A | G |
| rs55959471 | A | C | rs72810376 | T | C | rs3860270 | T | G |
| rs66825260 | C | G | 16:87013641 | A | C | rs3893739 | T | C |
| rs56201448 | A | G | 16:87013657 | T | C | 16:87069670 | A | C |
| rs4073239 | T | C | 16:87013678 | C | G | 16:87069805 | T | C |
| rs4072137 | T | C | rs117669610 | T | C | rs3901356 | T | G |
| rs7206179 | A | G | rs12929460 | A | G | rs112909154 | C | G |
| rs8062104 | T | G | rs12931207 | A | G | rs79551470 | T | C |
| rs117654548 | T | C | rs72810381 | A | G | rs3901357 | A | C |
| rs117166946 | T | C | rs9938781 | A | T | rs7194540 | T | C |
| rs77554460 | T | C | rs11648618 | T | C | rs17836043 | T | C |
| rs11860540 | C | G | rs11642297 | T | C | rs7195346 | A | G |
| rs7193141 | C | G | rs184680167 | A | C | rs72813927 | A | G |
| rs7201095 | T | C | rs9925922 | A | G | rs72813928 | A | C |
| rs11648463 | T | C | 16:87015610 | A | G | rs77924490 | A | G |
| rs60365364 | T | C | 16:87015617 | A | G | rs77755015 | A | G |
| rs73257030 | T | C | rs72628295 | A | T | rs1559335 | C | G |
| rs11642315 | A | G | rs72810386 | A | G | rs1011135 | A | G |
| rs8053225 | C | G | rs4843500 | T | C | rs2161645 | C | G |
| rs11643451 | T | C | rs8059711 | C | G | rs72813930 | T | G |
| rs11642398 | A | G | rs72628296 | T | C | 16:87073550 | A | C |
| rs73257036 | A | G | rs77239450 | T | C | rs721544 | A | G |
| rs35569067 | T | C | rs72810388 | T | C | rs116686582 | A | G |
| rs61639810 | C | G | rs62042554 | T | C | rs4066744 | T | C |
| rs59928952 | A | G | rs139161501 | A | C | rs79702534 | T | C |
| rs35012682 | A | G | rs11648927 | T | G | rs60967188 | A | G |
| rs56927929 | A | G | rs4843501 | C | G | rs113084920 | A | G |
| rs34528639 | A | G | rs62042555 | A | G | rs12446707 | A | C |
| rs73257039 | A | T | rs59245478 | A | G | rs79328787 | T | C |
| rs145991402 | T | C | rs58083962 | C | G | rs72813933 | T | G |
| rs34790797 | C | G | rs11117237 | A | C | rs7192841 | T | C |
| rs8052432 | C | G | rs12933656 | C | G | rs61399937 | A | G |
| rs34538350 | C | G | rs140719399 | C | G | rs112912155 | C | G |
| rs8056157 | A | G | rs77617632 | A | G | rs72813937 | C | G |
| rs8056328 | C | G | rs12921148 | T | C | rs4066743 | A | C |
| rs77379290 | A | G | rs149229313 | T | C | rs79992957 | T | G |
| rs8058128 | C | G | rs116851347 | A | C | rs72813939 | C | G |
| rs11644190 | T | C | rs4072201 | A | G | rs11647733 | A | G |
| rs143248065 | A | G | rs112092967 | T | C | rs7200290 | A | G |
| rs58618909 | A | T | rs4605172 | C | G | rs7199773 | T | C |
| rs12597884 | T | G | rs28405967 | A | G | rs7187668 | T | C |
| rs117301054 | T | C | rs4473193 | A | G | rs76231178 | T | C |
| rs12597972 | C | G | rs4074635 | A | G | rs9940627 | C | G |
| rs71390866 | T | G | rs117718488 | C | G | rs10779241 | A | G |
| rs66478303 | C | G | rs12444807 | A | G | rs8062884 | A | G |
| 16:86942931 | T | C | rs62042556 | T | G | rs6539999 | T | C |
| rs4077676 | A | G | rs11643143 | C | G | rs66720093 | A | G |
| rs117381857 | T | C | rs117441118 | A | G | rs67050666 | T | C |
| rs74036697 | T | G | rs9931460 | A | C | rs4378618 | A | T |
| rs28470675 | A | G | rs12918037 | C | G | rs72813943 | A | C |
| rs4074970 | A | C | rs66897481 | A | G | rs10863177 | T | G |
| rs59778474 | T | C | rs62042557 | T | C | rs78087318 | C | G |
| rs28588364 | T | C | rs67350156 | C | G | rs77878862 | C | G |
| rs11642848 | T | C | rs7201907 | A | G | rs72813945 | A | G |
| rs79863981 | T | C | rs7204035 | A | T | rs9928052 | A | G |
| rs11644371 | T | G | rs140737903 | T | C | rs150524744 | A | T |
| rs11117232 | C | G | rs56242802 | T | C | rs73246242 | A | C |
| rs75258357 | T | C | rs146993471 | A | G | rs3849233 | A | G |
| rs8054771 | T | C | rs4072133 | T | C | rs58371593 | T | C |
| rs61101584 | T | C | rs76876575 | T | C | rs3860268 | A | G |
| rs55786512 | A | G | rs75154802 | A | G | rs79182015 | T | C |
| rs57174997 | T | C | rs4074545 | T | C | rs12443938 | A | T |
| rs9925927 | C | G | rs4074544 | A | T | rs11117241 | A | C |
| rs4640189 | T | C | rs75937711 | C | G | rs34978691 | A | G |
| rs7500836 | T | C | rs4843204 | T | C | rs8062556 | T | C |
| rs9937401 | A | G | rs4843502 | A | C | rs11117242 | T | C |
| rs62042469 | T | C | rs62042558 | T | G | rs11641297 | T | G |
| rs10863172 | A | T | rs12925527 | A | G | rs11117243 | T | C |
| rs8045520 | T | C | rs7194455 | A | G | rs17769972 | C | G |
| rs8060943 | A | G | rs74939890 | T | C | rs4602049 | A | T |
| rs4624187 | T | C | rs4843205 | C | G | rs4496150 | A | C |
| rs71390868 | T | C | rs72812355 | T | C | rs1345118 | A | G |
| rs11648502 | T | C | rs4843206 | A | G | rs1965173 | A | G |
| rs8045065 | T | C | rs4843207 | T | C | rs1965172 | A | G |
| rs8060776 | C | G | rs4072858 | T | C | rs1345117 | A | G |
| rs8045280 | T | C | rs4843208 | A | T | rs16942600 | T | C |
| rs62042471 | C | G | rs4072857 | T | C | rs11643006 | C | G |
| rs113866193 | A | T | rs4843503 | T | C | rs17697331 | C | G |
| rs8045541 | A | C | rs4072856 | C | G | rs150083236 | T | C |
| rs6539984 | T | C | rs4072855 | C | G | rs8057767 | A | G |
| rs12933736 | T | C | rs4843209 | A | G | rs12449271 | T | C |
| rs117821282 | T | C | rs56364652 | A | G | rs146199257 | T | C |
| rs113077754 | A | G | rs4321203 | A | C | 16:87086729 | C | G |
| rs4340331 | A | T | rs72812359 | A | G | rs6540000 | C | G |
| rs74348516 | A | G | rs8063188 | A | C | rs77735684 | A | G |
| rs4488453 | A | G | rs8047948 | T | C | rs2416375 | C | G |
| rs79033967 | T | C | rs67788342 | A | G | rs895420 | A | G |
| rs28570535 | A | G | rs78800470 | A | T | rs7191444 | A | G |
| rs75502932 | A | G | rs7187934 | C | G | rs7190783 | T | C |
| rs11117234 | A | T | rs140025937 | A | G | rs7190936 | C | G |
| rs112436087 | A | G | rs78033689 | A | T | rs3909501 | A | T |
| rs148839421 | T | C | rs61281558 | T | C | rs3909502 | T | C |
| rs73258950 | A | G | rs11864228 | C | G | rs3909503 | T | C |
| rs58967411 | T | C | rs11860205 | A | G | rs58879762 | T | G |
| rs35794697 | C | G | rs11864040 | A | G | rs11643779 | A | T |
| rs13335005 | C | G | rs9934129 | T | C | rs11648465 | A | G |
| rs8043658 | A | G | rs9928537 | T | C | rs11648470 | T | G |
| rs58843785 | A | C | rs9928628 | A | T | rs11648671 | T | G |
| rs77490555 | A | G | rs12927039 | T | C | rs3849232 | T | C |
| rs79702723 | T | C | rs9926493 | A | G | rs72813954 | A | G |
| 16:86965965 | T | G | rs9928751 | T | C | rs79025839 | A | G |
| 16:86965989 | T | G | rs8054493 | A | C | rs7193281 | A | G |
| rs62636095 | C | G | rs77451386 | T | C | rs7192775 | C | G |
| rs78211164 | T | C | rs8054646 | T | C | rs12711471 | T | G |
| 16:86966434 | A | G | rs75304375 | A | G | | | |
| 16:86966484 | A | G | rs8060154 | T | C | | | |

TABLE 58
IPF transplant free survival variants associated with PUFIN1 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| 21:43429289 | A | G | 21:43446597 | A | G | 21:43461043 | A | G |
| 21:43429434 | A | G | 21:43446669 | A | G | 21:43461253 | A | G |
| rs907303666 | A | G | 21:43446687 | A | G | rs747606979 | T | C |
| 21:43429623 | A | G | rs570528396 | A | G | rs756692999 | A | T |
| 21:43429646 | A | G | rs56015857 | A | G | 21:43461809 | C | G |
| 21:43430837 | T | G | rs759575547 | A | G | 21:43461888 | T | C |
| 21:43430999 | T | G | 21:43447738 | T | G | rs1205978928 | A | G |
| rs562052587 | A | G | 21:43447746 | C | G | rs1028085905 | T | C |
| rs1359873392 | A | T | 21:43447862 | T | G | 21:43462379 | A | G |
| 21:43431575 | T | C | 21:43448159 | T | C | 21:43462542 | T | C |
| 21:43431830 | T | C | rs747069979 | T | C | rs762813528 | A | G |
| 21:43432254 | T | C | 21:43449024 | A | G | 21:43462712 | T | G |
| 21:43432297 | A | G | 21:43449213 | A | G | 21:43462868 | A | G |
| rs200753297 | A | G | 21:43449404 | C | G | 21:43463069 | C | G |
| 21:43432708 | T | C | 21:43449649 | T | C | rs1274119279 | T | G |
| 21:43432888 | T | C | 21:43449936 | T | C | rs762937005 | C | G |
| 21:43433131 | A | C | 21:43449967 | T | C | 21:43463562 | T | C |
| 21:43433157 | T | C | 21:43450149 | T | C | 21:43463599 | A | G |
| 21:43433161 | A | G | 21:43450314 | T | C | 21:43463749 | A | G |
| 21:43434141 | T | C | 21:43451056 | A | T | 21:43463801 | A | G |
| 21:43434177 | T | C | 21:43451141 | A | G | 21:43463948 | A | G |
| 21:43434243 | T | C | rs1319276776 | A | G | 21:43464179 | A | G |
| 21:43434303 | A | G | 21:43451368 | T | C | 21:43464182 | A | T |
| 21:43434362 | T | C | 21:43451397 | T | C | 21:43464217 | A | C |
| 21:43434601 | T | C | 21:43451586 | A | G | 21:43464361 | T | C |
| 21:43435406 | T | C | 21:43451659 | C | G | 21:43464392 | A | G |
| 21:43435555 | T | C | 21:43451868 | A | C | 21:43464413 | T | C |
| 21:43436503 | A | G | 21:43452154 | C | G | 21:43464736 | C | G |
| 21:43436630 | T | C | rs1462007946 | T | C | 21:43464924 | T | C |
| 21:43437886 | A | G | 21:43453558 | T | C | 21:43465427 | A | G |
| 21:43438017 | A | G | 21:43453662 | T | C | rs937269097 | A | G |
| 21:43438530 | A | G | 21:43453719 | T | C | 21:43465524 | T | C |
| 21:43438597 | A | G | 21:43453819 | T | C | 21:43465598 | T | C |
| 21:43438608 | T | C | 21:43453847 | T | C | 21:43465775 | A | G |
| 21:43438674 | A | C | 21:43453925 | T | C | 21:43465785 | A | C |
| 21:43438731 | A | G | rs924993617 | A | G | 21:43465831 | C | G |
| 21:43438748 | A | G | 21:43454215 | A | G | 21:43465928 | A | G |
| 21:43438761 | A | G | 21:43454252 | T | C | 21:43465959 | T | C |
| 21:43438766 | T | G | 21:43454304 | A | C | 21:43465984 | T | C |
| 21:43438781 | T | C | 21:43454402 | T | C | rs558655134 | T | C |
| 21:43438874 | A | G | 21:43454440 | T | C | rs866049712 | T | C |
| rs951994808 | A | G | 21:43454441 | A | G | 21:43466367 | T | C |
| 21:43438898 | T | G | 21:43454451 | T | C | 21:43466487 | T | C |
| 21:43438955 | A | G | 21:43454614 | T | C | rs1435482552 | A | C |
| 21:43439228 | C | G | 21:43454790 | A | G | 21:43466638 | A | G |
| 21:43439445 | C | G | 21:43454844 | T | C | 21:43466891 | A | G |
| 21:43439562 | A | G | 21:43454847 | A | G | 21:43466953 | A | G |
| rs534318351 | A | G | 21:43454885 | T | C | rs1008088471 | T | C |
| 21:43439679 | T | C | 21:43454888 | T | C | 21:43467225 | T | G |
| 21:43439681 | A | C | 21:43455125 | T | C | 21:43467264 | A | G |
| 21:43439732 | A | G | 21:43455168 | T | C | 21:43467498 | A | T |
| 21:43439820 | T | C | 21:43455180 | C | G | rs1396984718 | T | C |
| 21:43439827 | A | C | 21:43455291 | A | G | 21:43468115 | A | T |
| rs774985600 | A | G | 21:43455329 | A | G | rs1457943471 | T | C |
| 21:43440058 | A | G | 21:43455365 | T | C | rs1465728977 | T | G |
| 21:43440157 | T | C | 21:43455479 | A | G | rs75432668 | T | C |
| rs1223302049 | T | C | 21:43455741 | A | G | 21:43468830 | T | G |
| 21:43440257 | T | C | 21:43455850 | T | C | 21:43468836 | T | G |
| 21:43440838 | T | C | 21:43455949 | A | G | 21:43468984 | T | C |
| 21:43441250 | A | G | 21:43455957 | A | G | 21:43469094 | A | G |
| 21:43441315 | A | G | 21:43456048 | A | C | 21:43469183 | A | G |
| 21:43441479 | A | G | 21:43456164 | T | C | 21:43469570 | T | C |
| 21:43441554 | T | G | 21:43456812 | A | G | 21:43469650 | A | G |
| 21:43441834 | A | G | 21:43457124 | A | C | rs545168548 | A | G |
| rs200203860 | T | C | 21:43457469 | T | C | 21:43470113 | T | C |
| 21:43442049 | A | G | rs981259550 | T | G | 21:43470142 | T | C |
| 21:43442080 | A | G | 21:43457651 | A | T | 21:43470278 | A | T |
| rs1028782561 | C | G | 21:43457659 | T | C | 21:43470980 | A | G |
| 21:43442372 | T | C | 21:43457773 | T | C | rs1051714406 | A | C |
| 21:43442385 | T | C | 21:43457774 | A | G | 21:43471004 | A | C |
| rs1381471283 | A | G | 21:43457838 | A | C | 21:43471126 | A | G |
| 21:43442728 | A | G | 21:43457991 | T | C | 21:43471708 | T | C |
| rs1368045877 | A | C | rs1182994967 | A | G | 21:43471803 | T | C |
| 21:43442767 | A | G | rs1442275675 | A | G | 21:43471898 | A | G |
| 21:43442836 | A | G | 21:43458309 | T | C | 21:43471933 | T | C |
| 21:43442991 | T | C | 21:43458382 | T | C | 21:43471958 | C | G |
| 21:43443011 | T | C | 21:43458574 | A | C | 21:43472040 | A | G |
| 21:43443266 | A | C | 21:43458706 | T | C | 21:43472083 | A | G |
| 21:43443370 | A | G | 21:43458745 | A | G | 21:43472148 | A | G |
| 21:43443378 | A | G | 21:43459014 | A | T | 21:43472167 | T | C |
| 21:43443498 | T | C | 21:43459172 | T | C | 21:43472339 | T | C |
| 21:43444414 | C | G | 21:43459325 | T | C | rs566113852 | A | G |
| rs1227135914 | A | G | rs566458436 | T | G | 21:43472438 | A | G |
| rs542133500 | A | G | 21:43459958 | A | G | 21:43472588 | T | C |
| rs914881274 | T | C | 21:43459991 | T | C | 21:43472925 | T | C |
| 21:43444731 | T | C | 21:43460247 | T | C | rs1006521908 | C | G |
| 21:43445292 | A | G | 21:43460310 | A | G | 21:43473602 | A | C |
| rs1367366964 | A | C | 21:43460345 | T | C | 21:43473618 | T | C |
| 21:43446067 | T | C | rs556236327 | T | C | rs924823147 | A | G |
| 21:43446068 | A | G | 21:43460496 | A | G | rs756732695 | A | T |
| 21:43446474 | A | C | 21:43460608 | T | C | 21:43474163 | A | G |
| 21:43446487 | T | C | 21:43460852 | C | G | 21:43474929 | T | C |
| 21:43446546 | T | G | 21:43460912 | A | T | 21:43475390 | C | G |

TABLE 59
IPF transplant free survival variants associated with PUFIN2 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| rs894828979 | A | G | 15:39318750 | A | G | 15:39538624 | A | G |
| 15:39157332 | A | T | rs866770983 | T | C | 15:39538750 | T | G |
| rs1228048737 | T | C | 15:39319384 | A | T | 15:39538881 | T | C |
| 15:39157908 | A | G | 15:39319421 | A | G | 15:39538942 | A | C |
| 15:39158752 | T | G | 15:39319573 | A | G | rs949432890 | T | G |
| rs1178123021 | A | G | 15:39319915 | T | G | 15:39539555 | A | G |
| 15:39159021 | T | C | 15:39320011 | T | C | 15:39539621 | T | C |
| 15:39159221 | A | G | 15:39320924 | T | C | rs942541159 | T | C |
| 15:39159349 | A | G | 15:39320945 | A | C | 15:39539851 | A | T |
| 15:39159590 | T | C | 15:39321191 | A | G | 15:39539917 | T | C |
| 15:39159831 | T | C | rs182258573 | A | G | 15:39540343 | A | G |
| 15:39159867 | A | G | 15:39321589 | A | G | 15:39540578 | A | G |
| 15:39159955 | C | G | 15:39322212 | A | T | 15:39541379 | T | C |
| 15:39160069 | T | C | 15:39322422 | A | G | 15:39541390 | A | T |
| 15:39160313 | A | G | 15:39322865 | T | C | rs1161765371 | T | C |
| 15:39160362 | A | G | 15:39322975 | T | C | 15:39541532 | T | C |
| 15:39160431 | A | C | 15:39323778 | A | G | 15:39541533 | C | G |
| 15:39161011 | T | C | 15:39324626 | A | C | 15:39541631 | C | G |
| 15:39161147 | T | C | 15:39324646 | A | C | 15:39541864 | T | C |
| 15:39161381 | T | G | 15:39324690 | A | G | 15:39542033 | T | C |
| 15:39161727 | T | G | 15:39324880 | A | C | rs937955 | T | C |
| rs1385190898 | C | G | rs1274226450 | T | C | 15:39542265 | T | C |
| 15:39161964 | T | C | rs1303497178 | A | G | 15:39542375 | T | C |
| rs1320450739 | A | T | 15:39325487 | A | T | rs1448363301 | A | G |
| 15:39162337 | C | G | rs1480036450 | A | G | 15:39542919 | T | C |
| rs1235570860 | T | C | 15:39326839 | A | G | rs969409029 | T | C |
| 15:39162695 | A | C | 15:39327913 | A | G | rs955328535 | T | C |
| 15:39162913 | A | G | 15:39329919 | T | C | 15:39544129 | C | G |
| 15:39162990 | T | C | rs149600359 | T | C | 15:39544512 | T | C |
| rs1469612346 | T | C | 15:39330174 | T | C | rs956317979 | T | G |
| 15:39163669 | A | C | rs1359732081 | A | G | rs1409092516 | T | C |
| 15:39163687 | T | C | 15:39330791 | A | G | rs73387024 | A | G |
| 15:39163935 | T | C | rs1004896932 | A | G | 15:39545282 | A | G |
| 15:39164045 | T | C | 15:39331794 | T | G | 15:39545457 | T | C |
| 15:39164780 | A | G | 15:39333256 | T | C | 15:39545481 | A | G |
| 15:39164809 | A | G | 15:39333862 | A | C | 15:39546114 | A | G |
| 15:39164875 | A | G | 15:39334237 | T | C | 15:39546250 | A | G |
| 15:39164921 | A | C | 15:39334326 | T | C | rs80177059 | C | G |
| 15:39164983 | T | C | 15:39334387 | A | G | rs59249376 | C | G |
| 15:39165053 | A | G | 15:39334561 | T | C | 15:39546830 | T | C |
| 15:39165415 | C | G | 15:39335388 | A | G | 15:39547372 | A | G |
| 15:39165581 | T | C | 15:39335692 | T | C | 15:39547483 | T | C |
| 15:39165937 | T | C | 15:39335937 | T | C | 15:39547526 | A | C |
| 15:39166073 | T | C | 15:39338263 | A | G | 15:39547658 | A | G |
| 15:39166084 | A | C | rs73387489 | A | G | 15:39548327 | A | G |
| 15:39166417 | T | C | 15:39339450 | T | C | 15:39548420 | T | C |
| rs1039139264 | C | G | rs1237107069 | C | G | rs1293390602 | T | C |
| 15:39166735 | A | G | 15:39342128 | T | C | 15:39548742 | A | G |
| 15:39166885 | T | C | rs924291878 | A | G | 15:39548743 | C | G |
| 15:39167383 | A | G | 15:39342995 | A | G | 15:39549334 | A | T |
| rs368280268 | A | G | 15:39343032 | T | C | 15:39550557 | A | C |
| 15:39167916 | A | G | rs528777258 | T | C | 15:39550929 | T | C |
| rs971179827 | A | G | 15:39344267 | T | C | 15:39552068 | A | T |
| 15:39169241 | T | G | 15:39344759 | A | G | rs1041309131 | T | C |
| 15:39169695 | A | G | 15:39344929 | A | G | 15:39552699 | A | T |
| 15:39169698 | C | G | 15:39345346 | C | G | 15:39552754 | T | G |
| 15:39169750 | A | C | 15:39346000 | A | C | 15:39553167 | A | G |
| 15:39169779 | A | G | 15:39346077 | A | G | 15:39553976 | T | C |
| 15:39169786 | A | C | 15:39346118 | T | C | 15:39554097 | T | C |
| 15:39169885 | T | C | 15:39346437 | A | G | 15:39554614 | A | G |
| rs1439499168 | T | C | 15:39346474 | T | C | 15:39554618 | A | G |
| 15:39169960 | T | C | 15:39347039 | A | G | 15:39555182 | A | G |
| 15:39170014 | T | C | 15:39347046 | A | G | 15:39555439 | T | C |
| rs977256273 | T | C | rs1308815263 | A | G | 15:39555440 | A | G |
| rs1261621131 | A | G | rs1352851519 | T | C | 15:39555663 | T | C |
| 15:39170985 | T | C | rs566453792 | A | G | 15:39555993 | T | G |
| 15:39171059 | A | G | 15:39348606 | A | T | 15:39556231 | T | C |
| 15:39171614 | A | T | 15:39349187 | T | C | 15:39556329 | A | G |
| 15:39171742 | T | G | 15:39349762 | A | G | 15:39556435 | A | G |
| 15:39171746 | T | G | 15:39350160 | T | G | 15:39556482 | C | G |
| 15:39171762 | A | T | 15:39350584 | T | C | 15:39556796 | T | C |
| 15:39171851 | T | C | 15:39351386 | T | C | 15:39558386 | T | C |
| 15:39171857 | T | C | 15:39351937 | A | T | 15:39558430 | A | C |
| 15:39172049 | A | G | rs1302282442 | T | G | rs574145853 | A | G |
| 15:39172185 | A | G | 15:39352587 | T | G | 15:39559065 | A | G |
| 15:39172642 | T | C | 15:39352927 | T | C | rs1411341155 | T | C |
| 15:39173107 | C | G | 15:39353319 | A | C | 15:39559439 | A | G |
| 15:39173266 | T | C | rs866274592 | C | G | rs146661125 | T | G |
| 15:39173268 | A | G | 15:39354292 | T | C | 15:39559675 | T | C |
| 15:39173422 | T | C | 15:39354310 | C | G | rs374228327 | A | T |
| 15:39173423 | A | G | rs1227892432 | C | G | rs1364783544 | A | G |
| 15:39173568 | A | C | rs951992416 | A | G | 15:39560371 | T | C |
| 15:39173911 | C | G | 15:39355716 | T | C | 15:39560740 | T | C |
| 15:39173982 | A | T | 15:39355732 | T | C | rs1389693399 | A | G |
| 15:39174082 | T | C | rs1035938551 | A | C | 15:39561038 | A | T |
| 15:39174375 | A | G | 15:39355860 | A | G | 15:39561203 | T | C |
| 15:39174902 | A | G | rs1179892613 | T | C | 15:39561242 | A | G |
| 15:39175054 | C | G | 15:39356271 | A | C | 15:39561381 | T | C |
| 15:39175062 | C | G | rs537135781 | T | C | 15:39561721 | A | G |
| rs889467621 | A | G | 15:39356753 | A | G | rs1358681094 | T | G |
| rs970083027 | C | G | 15:39357211 | A | T | 15:39562498 | A | G |
| rs1056533468 | T | C | rs1207881058 | T | C | 15:39562502 | T | C |
| 15:39176291 | T | C | 15:39357403 | T | C | 15:39562712 | A | C |
| 15:39176365 | A | G | rs1421887694 | A | G | 15:39562953 | T | C |
| rs971791005 | A | G | 15:39358204 | T | C | 15:39562972 | T | G |
| 15:39176921 | A | T | 15:39358388 | T | C | 15:39563357 | T | C |
| 15:39176964 | A | G | rs892372556 | A | G | 15:39563428 | T | C |
| rs58154208 | T | G | 15:39359275 | T | C | 15:39563463 | T | G |
| 15:39177133 | T | C | rs1483039626 | A | G | 15:39563871 | T | G |
| rs978933971 | A | C | 15:39359778 | C | G | 15:39564293 | A | G |
| rs183665955 | A | T | 15:39360169 | T | C | 15:39564294 | T | C |
| 15:39178144 | T | G | 15:39360198 | A | G | 15:39564767 | A | G |
| 15:39178147 | A | G | 15:39360615 | A | T | 15:39566195 | T | C |
| 15:39178621 | A | G | 15:39360778 | A | T | 15:39566710 | A | C |
| 15:39178754 | A | G | 15:39360797 | A | G | 15:39567305 | A | C |
| 15:39178788 | A | G | 15:39360835 | A | C | 15:39568254 | A | G |
| 15:39178813 | T | G | 15:39360845 | A | G | 15:39568381 | T | C |
| 15:39179285 | T | C | 15:39360860 | T | C | 15:39568747 | C | G |
| 15:39179307 | A | G | rs892620708 | T | G | 15:39568859 | A | T |
| 15:39179321 | A | G | 15:39361986 | T | C | 15:39569250 | T | C |
| 15:39179357 | T | C | 15:39362142 | C | G | 15:39569335 | A | C |
| rs1038692235 | C | G | rs1234190063 | T | C | rs907223538 | T | G |
| 15:39179436 | T | C | rs771081711 | A | G | 15:39570051 | T | C |
| 15:39179446 | T | C | 15:39362834 | T | C | 15:39570229 | A | G |
| 15:39179449 | A | T | 15:39362902 | T | C | 15:39570244 | T | C |
| 15:39179518 | A | T | rs1470969625 | A | C | rs149257880 | T | C |
| rs529246748 | A | G | 15:39363256 | A | T | 15:39570749 | A | G |
| rs185889147 | A | T | 15:39363684 | T | C | rs1186504978 | A | G |
| 15:39179819 | T | G | 15:39363702 | A | G | 15:39571220 | A | G |
| 15:39179918 | A | C | 15:39363736 | A | G | rs72725044 | A | G |
| 15:39180047 | T | C | rs1412139446 | A | C | rs1022612903 | A | G |
| 15:39180207 | A | G | 15:39364078 | A | G | 15:39571293 | A | G |
| 15:39180595 | A | C | 15:39364173 | T | C | 15:39571323 | T | C |
| rs1208543203 | T | C | rs753899491 | A | G | 15:39572146 | A | G |
| 15:39182392 | A | C | 15:39364239 | A | G | 15:39572278 | T | C |
| rs1223747917 | C | G | 15:39364268 | T | C | 15:39572353 | A | G |
| 15:39182580 | A | G | 15:39364789 | A | G | 15:39573307 | T | C |
| 15:39182675 | A | T | 15:39364952 | A | G | 15:39573535 | A | G |
| 15:39182687 | T | C | 15:39365070 | T | C | 15:39573686 | A | G |
| 15:39182784 | A | G | 15:39365137 | A | G | 15:39574111 | T | G |
| rs1368122039 | A | G | rs989380250 | A | G | 15:39574914 | A | C |
| 15:39182832 | T | C | 15:39365195 | A | G | 15:39575166 | T | C |
| 15:39183106 | C | G | 15:39365230 | A | C | 15:39575219 | A | G |
| 15:39183370 | A | G | 15:39365290 | A | C | 15:39575394 | T | C |
| 15:39183430 | T | C | rs1371013044 | A | C | 15:39575638 | A | C |
| 15:39183551 | A | C | 15:39365414 | T | C | 15:39576560 | A | C |
| 15:39183592 | A | G | 15:39365456 | A | G | 15:39576705 | T | C |
| 15:39183676 | A | G | 15:39365505 | C | G | 15:39577100 | T | C |
| 15:39183729 | A | G | rs930154326 | T | C | 15:39577347 | T | C |
| 15:39184039 | C | G | 15:39365622 | A | G | 15:39577814 | A | T |
| 15:39184601 | A | G | 15:39365851 | A | G | 15:39577821 | A | C |
| 15:39184711 | T | C | 15:39365884 | T | C | 15:39577879 | A | C |
| 15:39184926 | T | G | 15:39366499 | T | C | 15:39577925 | A | C |
| 15:39186568 | A | G | rs568569971 | T | C | 15:39578531 | T | C |
| 15:39187185 | A | G | 15:39366792 | A | G | 15:39578635 | T | C |
| 15:39187441 | A | C | 15:39366899 | A | C | 15:39579082 | T | C |
| 15:39187846 | A | C | rs1371201699 | A | T | rs908981632 | C | G |
| 15:39187900 | A | T | 15:39367052 | A | C | 15:39579430 | T | C |
| 15:39188148 | T | G | 15:39367068 | T | C | 15:39579485 | A | G |
| 15:39188204 | T | C | rs1041522742 | T | C | 15:39580180 | A | G |
| 15:39188421 | A | C | 15:39367313 | A | G | 15:39580264 | A | G |
| rs994093137 | T | C | rs901379511 | T | C | 15:39580395 | A | G |
| 15:39188529 | T | C | 15:39367535 | A | G | 15:39580746 | A | G |
| rs919228953 | A | G | rs190905288 | A | G | 15:39580985 | T | C |
| rs1248079824 | T | G | 15:39367606 | A | T | rs1015178023 | T | C |
| 15:39188787 | T | C | 15:39367646 | T | C | 15:39581271 | T | C |
| 15:39188965 | T | C | 15:39367856 | T | C | 15:39581586 | A | G |
| 15:39189053 | T | C | 15:39367923 | A | G | 15:39582260 | T | C |
| 15:39189478 | T | C | rs913154009 | A | G | rs749046805 | C | G |
| 15:39189692 | A | G | 15:39368024 | T | C | rs1297392427 | T | C |
| 15:39190060 | T | C | 15:39368342 | C | G | 15:39582769 | A | T |
| rs988006801 | A | G | rs1355548851 | A | G | 15:39582770 | C | G |
| 15:39190790 | C | G | 15:39368667 | C | G | 15:39583120 | T | G |
| 15:39191412 | T | C | 15:39369043 | T | C | 15:39584031 | T | G |
| 15:39191873 | C | G | 15:39369397 | A | C | 15:39584123 | T | C |
| 15:39192017 | A | G | 15:39369451 | A | G | 15:39584159 | C | G |
| 15:39192232 | T | C | 15:39369574 | T | C | 15:39584747 | T | C |
| rs552024813 | A | G | 15:39369688 | A | G | rs1275552796 | A | C |
| 15:39192678 | T | C | 15:39369935 | T | C | 15:39585024 | T | C |
| 15:39192806 | A | G | 15:39370355 | T | C | 15:39585032 | A | G |
| 15:39193086 | T | C | 15:39370374 | A | G | 15:39585277 | C | G |
| 15:39193183 | T | C | 15:39370406 | A | C | rs1326315492 | T | C |
| 15:39193184 | T | C | 15:39370471 | T | G | 15:39585383 | A | G |
| 15:39193242 | T | C | 15:39370517 | A | G | 15:39585428 | A | T |
| rs957143281 | A | G | 15:39370520 | A | C | 15:39585535 | T | C |
| 15:39193265 | A | T | rs182772816 | T | C | rs917672293 | T | G |
| rs1036361092 | T | C | 15:39370567 | A | G | 15:39585703 | A | G |
| 15:39193338 | C | G | 15:39370638 | A | C | rs935400502 | T | C |
| rs994917231 | T | G | 15:39370742 | C | G | 15:39585865 | A | G |
| 15:39193593 | C | G | 15:39370877 | A | G | 15:39585893 | T | C |
| 15:39194040 | A | C | 15:39370929 | T | C | 15:39585974 | T | G |
| 15:39194084 | C | G | 15:39370956 | A | G | 15:39586152 | T | C |
| 15:39194310 | T | C | rs182975121 | A | G | 15:39586354 | T | C |
| 15:39194778 | A | G | 15:39371190 | T | G | rs1359083762 | T | C |
| rs1006320289 | A | G | 15:39371206 | A | T | 15:39586557 | A | G |
| 15:39195053 | A | G | rs901545645 | T | C | 15:39586694 | T | C |
| 15:39195367 | T | C | 15:39371299 | T | G | rs953832443 | T | G |
| 15:39195441 | A | G | 15:39371345 | T | G | 15:39587121 | C | G |
| rs1330141252 | T | C | rs114798820 | A | G | 15:39587293 | C | G |
| rs564205113 | A | G | 15:39371768 | T | C | 15:39587337 | T | C |
| 15:39196664 | A | G | 15:39372060 | T | C | rs376997190 | T | C |
| 15:39197015 | A | G | rs891404297 | T | C | 15:39587664 | C | G |
| 15:39197926 | T | C | 15:39372238 | C | G | rs1271146540 | T | C |
| rs1013315038 | T | C | 15:39372394 | T | C | rs1030689806 | A | T |
| rs1431959214 | A | G | rs963651617 | A | G | rs1217517264 | A | G |
| 15:39198444 | T | C | 15:39372766 | A | G | 15:39588417 | T | C |
| 15:39198688 | A | G | 15:39373654 | A | T | rs756948328 | T | C |
| 15:39198839 | T | C | rs1350232365 | A | T | rs1223692427 | C | G |
| 15:39198985 | T | C | rs1399138376 | C | G | rs1404325090 | A | G |
| 15:39199595 | T | C | 15:39373991 | T | C | 15:39588964 | T | G |
| 15:39199610 | A | G | 15:39374094 | T | C | rs1206575648 | C | G |
| rs1209761451 | C | G | 15:39374365 | A | C | 15:39589126 | T | C |
| 15:39200031 | C | G | 15:39374715 | A | T | rs1450043238 | T | C |
| 15:39200117 | T | C | 15:39374881 | A | G | 15:39589195 | A | G |
| 15:39200457 | C | G | rs112831552 | A | G | 15:39589246 | A | G |
| 15:39200686 | T | G | 15:39375151 | T | C | 15:39589280 | T | C |
| 15:39201040 | A | T | 15:39375211 | A | G | rs971669835 | C | G |
| 15:39201066 | A | G | 15:39375300 | A | G | 15:39589704 | T | C |
| 15:39201431 | A | G | 15:39375343 | T | C | 15:39589733 | A | C |
| 15:39201719 | T | C | 15:39375657 | T | C | 15:39589738 | A | G |
| 15:39201815 | A | C | 15:39375743 | A | G | 15:39590048 | A | G |
| 15:39202496 | A | G | 15:39375751 | A | G | 15:39590057 | A | G |
| 15:39202530 | T | C | 15:39376227 | C | G | 15:39590488 | T | G |
| 15:39203056 | A | T | 15:39376501 | T | C | 15:39590500 | T | C |
| rs1427207811 | T | C | 15:39376697 | T | G | 15:39591148 | T | C |
| rs1227516155 | C | G | 15:39376875 | A | C | 15:39591344 | A | G |
| 15:39204139 | C | G | 15:39377000 | C | G | 15:39591497 | T | G |
| 15:39204616 | T | G | 15:39377031 | T | C | 15:39591957 | A | G |
| rs1477443252 | A | G | 15:39377465 | A | G | 15:39592002 | C | G |
| 15:39204937 | T | C | 15:39377572 | A | G | 15:39592155 | A | G |
| rs1371900993 | A | C | 15:39377736 | T | C | 15:39592183 | T | C |
| 15:39205447 | A | G | 15:39378139 | A | G | rs1409601268 | A | T |
| 15:39205468 | T | C | 15:39378301 | T | C | 15:39592538 | T | G |
| 15:39206294 | A | T | 15:39378582 | A | G | 15:39592554 | T | C |
| 15:39206339 | T | C | 15:39378971 | T | C | 15:39592655 | T | C |
| 15:39206509 | T | C | 15:39379069 | A | G | 15:39592695 | A | G |
| 15:39206635 | A | G | 15:39379074 | T | C | rs764699626 | T | C |
| rs1208238594 | T | C | rs1283819087 | A | G | 15:39593078 | A | T |
| rs1464762025 | A | C | 15:39379355 | A | T | 15:39593106 | T | C |
| rs1025320671 | A | T | 15:39379475 | A | G | rs1053689953 | A | G |
| rs72739361 | A | G | 15:39380027 | C | G | 15:39593437 | T | G |
| rs894076664 | T | C | rs1251631709 | C | G | 15:39593579 | T | G |
| 15:39207763 | A | C | rs561562822 | A | G | rs1330273716 | C | G |
| 15:39207837 | A | T | 15:39380526 | T | C | 15:39594247 | A | T |
| 15:39208257 | T | C | 15:39380647 | A | T | 15:39594628 | A | G |
| 15:39208442 | A | C | rs942642119 | A | G | 15:39594681 | T | C |
| 15:39208553 | A | G | 15:39381311 | A | T | 15:39594826 | A | T |
| 15:39208765 | A | T | 15:39381486 | T | C | rs961423458 | T | C |
| 15:39209095 | T | C | 15:39381672 | A | G | 15:39595301 | T | C |
| 15:39209373 | C | G | 15:39381731 | A | G | 15:39595340 | A | G |
| rs1041939175 | A | G | 15:39381785 | T | C | 15:39595347 | A | G |
| 15:39209585 | A | G | rs766965274 | T | C | 15:39595429 | A | G |
| 15:39209855 | T | C | 15:39382087 | A | G | 15:39595577 | T | C |
| 15:39210056 | A | G | 15:39382250 | T | C | 15:39595684 | A | G |
| 15:39210169 | T | C | 15:39382665 | T | C | rs777787926 | A | G |
| 15:39210294 | A | T | 15:39382946 | T | C | rs908919227 | T | C |
| rs1459314135 | C | G | 15:39383194 | A | G | rs1017746076 | C | G |
| 15:39211239 | T | C | 15:39383337 | A | G | 15:39597034 | T | C |
| 15:39211405 | A | G | 15:39383421 | T | C | rs1274149815 | T | G |
| rs866076316 | A | G | 15:39383570 | A | G | rs1216891158 | T | C |
| 15:39211729 | C | G | 15:39383597 | A | G | rs942276045 | C | G |
| 15:39212126 | A | T | 15:39383860 | T | G | 15:39597432 | T | G |
| rs370057638 | A | G | rs953302193 | T | C | 15:39597461 | T | G |
| rs1374833498 | T | C | rs1424414790 | T | C | rs993107780 | A | G |
| rs1165921884 | A | G | rs574013580 | A | G | 15:39597702 | T | G |
| 15:39213142 | A | G | 15:39384805 | T | C | 15:39597883 | A | C |
| 15:39213172 | T | G | 15:39385506 | A | G | 15:39598016 | A | G |
| 15:39213181 | T | C | 15:39385545 | T | C | 15:39598111 | A | T |
| 15:39213734 | T | C | 15:39385691 | T | C | 15:39598133 | A | G |
| 15:39213851 | A | C | rs1389280675 | A | C | 15:39598392 | A | C |
| 15:39213858 | A | G | rs1319178877 | A | T | 15:39598394 | T | G |
| 15:39214245 | T | C | 15:39387298 | T | C | 15:39598407 | T | C |
| 15:39215301 | T | C | rs1000592745 | T | C | 15:39598529 | A | G |
| rs1251478483 | A | G | 15:39387518 | A | G | 15:39598596 | T | C |
| 15:39215618 | A | G | 15:39387629 | T | G | 15:39598672 | T | C |
| 15:39215895 | T | C | 15:39388031 | T | C | 15:39598843 | A | G |
| 15:39215902 | T | C | rs1015833579 | T | C | 15:39598965 | A | G |
| rs969663592 | A | G | rs192912418 | T | C | rs1020633894 | T | C |
| 15:39216330 | A | G | 15:39388654 | A | C | 15:39599319 | T | C |
| 15:39216432 | T | G | 15:39388882 | C | G | 15:39599667 | T | G |
| 15:39216471 | A | G | rs1299128845 | A | G | 15:39599818 | T | C |
| 15:39216950 | A | G | 15:39389419 | T | C | 15:39599878 | A | G |
| rs1336828823 | A | G | 15:39390021 | T | C | 15:39600026 | T | C |
| 15:39217159 | A | G | 15:39390253 | A | G | 15:39600291 | T | C |
| 15:39217606 | C | G | 15:39390286 | A | G | 15:39600364 | A | G |
| 15:39218101 | T | G | 15:39390479 | T | C | 15:39600386 | C | G |
| 15:39218128 | A | G | 15:39390740 | A | G | 15:39600572 | T | C |
| 15:39218132 | T | C | 15:39390938 | T | C | 15:39601513 | C | G |
| 15:39218481 | T | C | rs888398279 | T | C | rs1415485330 | A | G |
| 15:39218504 | A | G | 15:39391520 | T | C | rs763287582 | A | G |
| rs1033961355 | T | C | 15:39391704 | A | G | 15:39603701 | A | G |
| 15:39218874 | A | G | 15:39394081 | T | C | rs1272854499 | A | G |
| 15:39219009 | C | G | rs1045803496 | A | G | 15:39604140 | A | G |
| 15:39219116 | A | T | 15:39395507 | A | T | 15:39604292 | T | C |
| 15:39219362 | A | C | 15:39395544 | T | C | 15:39604340 | T | C |
| 15:39219412 | A | G | 15:39396761 | T | C | 15:39604391 | T | C |
| 15:39219416 | C | G | rs1309550200 | A | G | 15:39604529 | T | C |
| rs1261310766 | A | T | 15:39397306 | A | G | 15:39604835 | T | C |
| rs1169655308 | A | G | 15:39397325 | A | G | 15:39605344 | A | G |
| 15:39219505 | T | C | 15:39397579 | T | C | rs922084362 | A | C |
| 15:39219704 | T | C | 15:39397650 | T | G | 15:39605404 | T | C |
| 15:39219791 | A | G | 15:39397761 | C | G | 15:39605465 | T | C |
| rs112624316 | T | C | 15:39397870 | T | C | 15:39605493 | T | C |
| 15:39220258 | T | C | rs955220619 | T | C | 15:39605726 | A | G |
| 15:39220743 | A | G | 15:39398813 | T | G | 15:39605728 | T | C |
| rs1465671242 | C | G | 15:39399154 | C | G | 15:39605946 | T | C |
| 15:39220787 | T | C | 15:39399430 | T | C | 15:39606026 | T | C |
| 15:39221020 | A | C | 15:39399436 | A | C | rs1020433460 | A | T |
| rs1408730289 | A | G | 15:39399758 | A | G | 15:39606442 | A | G |
| rs1302672362 | A | C | 15:39400204 | T | C | rs1320352515 | T | C |
| 15:39221189 | T | C | 15:39400332 | T | C | rs1045909340 | A | G |
| 15:39221262 | A | C | 15:39400958 | A | G | rs940087819 | T | C |
| 15:39221324 | T | C | rs1316190182 | A | G | 15:39607401 | T | C |
| 15:39221398 | A | G | 15:39401172 | T | C | 15:39607442 | A | G |
| 15:39221630 | A | G | 15:39401850 | A | G | 15:39607514 | A | G |
| 15:39221710 | A | G | 15:39402179 | A | T | 15:39607589 | A | C |
| 15:39221817 | C | G | 15:39402251 | A | G | rs938322441 | A | G |
| 15:39221992 | T | G | rs1396877448 | T | C | 15:39607665 | A | G |
| rs1374756615 | T | C | 15:39403211 | A | T | 15:39608192 | A | T |
| 15:39222614 | T | C | 15:39403450 | T | C | 15:39608391 | T | C |
| 15:39222898 | C | G | rs190647755 | A | T | 15:39609115 | T | C |
| 15:39222988 | A | T | 15:39404337 | T | C | 15:39609118 | T | C |
| 15:39223082 | T | C | 15:39404393 | T | C | 15:39609167 | A | C |
| 15:39223143 | A | G | 15:39405070 | A | G | 15:39609296 | T | C |
| 15:39223600 | T | G | 15:39405081 | A | G | 15:39609393 | T | C |
| rs1401075231 | A | G | 15:39405148 | A | G | 15:39609598 | T | C |
| 15:39224137 | T | C | 15:39405690 | T | C | rs745391454 | C | G |
| 15:39224334 | A | G | 15:39405829 | A | T | 15:39609907 | A | G |
| 15:39224711 | A | G | 15:39406150 | A | G | 15:39610231 | A | T |
| 15:39224886 | T | C | 15:39406243 | A | G | 15:39610292 | A | G |
| 15:39224897 | A | C | 15:39406269 | A | G | 15:39610324 | C | G |
| rs568970815 | C | G | rs1330492020 | A | G | 15:39610360 | T | C |
| 15:39224986 | T | G | 15:39406778 | T | G | 15:39610373 | A | C |
| 15:39225365 | A | C | 15:39406791 | C | G | 15:39610377 | T | C |
| 15:39225945 | T | C | 15:39406827 | C | G | 15:39610666 | A | T |
| 15:39226012 | A | G | rs959128005 | T | C | 15:39610675 | T | C |
| 15:39226219 | A | G | 15:39407035 | T | C | 15:39610941 | A | G |
| 15:39226550 | A | G | 15:39407191 | T | G | 15:39611046 | A | G |
| 15:39226813 | T | C | 15:39407280 | A | C | rs1461716800 | T | C |
| 15:39227458 | A | C | 15:39407364 | A | G | 15:39611101 | T | C |
| 15:39227541 | T | C | 15:39407534 | T | C | rs1161242821 | A | T |
| 15:39227595 | C | G | 15:39407597 | A | G | rs535863496 | T | C |
| 15:39227644 | C | G | 15:39407621 | A | T | rs1327402327 | T | C |
| 15:39227715 | C | G | rs537249723 | A | C | rs1284531783 | A | G |
| 15:39227734 | A | G | 15:39408092 | A | C | 15:39611842 | T | C |
| 15:39228122 | T | C | rs1030938994 | A | G | 15:39611891 | A | G |
| 15:39228203 | C | G | 15:39408611 | A | G | 15:39611904 | A | G |
| 15:39228471 | A | G | 15:39408765 | T | C | 15:39612213 | A | G |
| 15:39228587 | T | C | rs1258490913 | A | G | rs139384283 | A | T |
| 15:39228610 | T | C | 15:39408907 | A | G | 15:39612264 | T | C |
| 15:39229103 | T | C | 15:39409097 | A | G | rs915243528 | A | G |
| 15:39229276 | A | G | rs1367913951 | T | G | 15:39612580 | A | T |
| 15:39229446 | T | C | 15:39409675 | T | C | 15:39613043 | T | C |
| 15:39229447 | A | C | 15:39410177 | A | T | 15:39613234 | T | C |
| 15:39229553 | A | G | 15:39410330 | T | C | rs1367789868 | A | G |
| 15:39229611 | A | G | 15:39411077 | A | T | 15:39613267 | T | C |
| 15:39229740 | A | G | 15:39411773 | A | C | 15:39613339 | A | G |
| 15:39229752 | A | G | 15:39412123 | A | C | 15:39613763 | T | C |
| rs564860175 | A | G | 15:39412350 | A | G | rs952182733 | A | G |
| rs1248502560 | T | C | 15:39412569 | T | C | 15:39613830 | T | C |
| 15:39229948 | T | C | 15:39412590 | T | C | 15:39613859 | A | G |
| 15:39230387 | T | C | 15:39412758 | T | C | 15:39614055 | A | G |
| rs1030122235 | C | G | rs1184465527 | A | T | 15:39614064 | T | G |
| 15:39230938 | T | C | rs994883307 | C | G | rs1345460438 | A | G |
| rs1013531357 | T | C | 15:39413196 | A | G | 15:39614238 | T | C |
| 15:39231397 | A | G | rs1383104432 | C | G | 15:39614315 | T | C |
| 15:39231457 | T | C | 15:39413575 | A | G | 15:39614331 | A | G |
| rs1431294407 | A | T | 15:39413773 | A | G | 15:39614333 | A | T |
| 15:39232829 | A | G | 15:39414306 | C | G | rs1282997825 | T | C |
| rs894596360 | A | G | 15:39414639 | A | G | 15:39614736 | T | C |
| 15:39233253 | C | G | 15:39416713 | C | G | 15:39615016 | A | G |
| 15:39233452 | A | G | rs879578183 | A | G | 15:39615145 | A | C |
| 15:39233486 | A | C | 15:39417307 | T | C | 15:39615418 | A | G |
| 15:39233794 | C | G | 15:39419181 | A | C | 15:39615599 | C | G |
| 15:39233814 | T | G | 15:39419538 | A | T | rs993884460 | A | G |
| rs1002517585 | T | C | 15:39421632 | T | C | 15:39615636 | T | C |
| 15:39233941 | T | C | 15:39423132 | T | C | 15:39615903 | T | C |
| 15:39233989 | C | G | 15:39424370 | T | C | rs1254022670 | C | G |
| 15:39234305 | T | C | rs1419877584 | A | G | 15:39616189 | T | C |
| 15:39234334 | T | C | 15:39426121 | T | C | 15:39616279 | C | G |
| 15:39234425 | T | C | 15:39426516 | A | G | 15:39616390 | A | G |
| 15:39234496 | A | G | 15:39426899 | T | C | 15:39616715 | T | G |
| 15:39234895 | T | C | 15:39427665 | A | G | 15:39617142 | A | T |
| 15:39235110 | A | G | 15:39428081 | T | G | 15:39617143 | A | T |
| rs550821273 | C | G | 15:39428162 | T | C | 15:39617262 | A | G |
| 15:39235357 | A | C | 15:39428514 | T | C | 15:39617295 | T | C |
| 15:39235596 | T | G | rs1368472770 | T | C | rs577264599 | A | G |
| 15:39235700 | T | C | rs914473670 | C | G | rs138630370 | A | G |
| 15:39236171 | A | T | rs568886788 | C | G | 15:39617951 | T | G |
| rs371625302 | T | C | 15:39430806 | T | C | 15:39618885 | T | C |
| 15:39237182 | T | C | 15:39430914 | A | C | 15:39618899 | T | C |
| 15:39237677 | A | T | 15:39432056 | T | C | rs978987992 | A | C |
| 15:39237995 | T | G | 15:39432772 | T | G | 15:39619458 | T | C |
| 15:39238819 | A | G | 15:39433297 | T | C | 15:39619621 | A | G |
| rs972082602 | T | G | 15:39433298 | A | T | rs780785908 | C | G |
| 15:39239014 | A | C | 15:39433947 | A | G | 15:39619957 | T | C |
| 15:39239162 | T | C | 15:39434363 | A | G | 15:39619980 | T | C |
| rs1295228977 | T | C | 15:39434458 | A | G | 15:39620028 | A | G |
| rs1381563320 | A | G | 15:39434616 | A | G | 15:39620247 | A | G |
| rs927518507 | A | G | 15:39434682 | T | C | 15:39620334 | T | C |
| 15:39240110 | A | C | 15:39434980 | T | C | 15:39620527 | A | G |
| rs776317940 | T | C | 15:39435145 | T | C | 15:39620602 | C | G |
| 15:39240759 | A | G | 15:39435280 | A | G | rs1041724652 | T | C |
| 15:39241067 | T | C | rs1354046968 | A | G | 15:39620854 | T | G |
| 15:39241068 | A | G | rs1486195961 | A | T | 15:39620866 | T | G |
| 15:39241109 | T | G | 15:39436303 | C | G | 15:39620920 | A | T |
| 15:39241180 | A | C | rs562123589 | T | C | 15:39621418 | A | G |
| 15:39241566 | C | G | 15:39436707 | T | C | 15:39621773 | T | C |
| rs973385490 | C | G | rs954219147 | A | C | 15:39622468 | T | G |
| 15:39241852 | A | C | 15:39437713 | A | G | 15:39622472 | A | G |
| 15:39242724 | A | T | 15:39438461 | A | C | 15:39622593 | T | C |
| 15:39243426 | T | C | rs902504992 | A | G | 15:39622615 | T | C |
| rs1219132454 | A | G | 15:39438795 | A | G | 15:39622687 | T | C |
| 15:39243458 | C | G | 15:39439200 | T | C | 15:39623097 | T | C |
| 15:39243482 | T | C | 15:39441064 | A | G | 15:39623509 | T | C |
| 15:39243499 | C | G | 15:39441229 | T | C | rs1425877139 | T | C |
| 15:39243639 | T | C | 15:39441256 | A | G | rs1455015180 | A | G |
| 15:39244786 | T | C | rs537988974 | A | G | 15:39623847 | C | G |
| rs1214531750 | A | T | 15:39441862 | A | G | 15:39624268 | A | C |
| 15:39245900 | A | G | rs1327238629 | A | G | 15:39624418 | T | C |
| 15:39246101 | A | G | 15:39442104 | A | G | 15:39624725 | A | G |
| rs983995811 | T | G | 15:39442474 | A | G | rs746620023 | T | C |
| rs190640884 | A | G | 15:39442635 | T | C | 15:39624864 | C | G |
| 15:39246712 | A | G | rs564223863 | T | C | 15:39625000 | A | G |
| 15:39246754 | A | G | 15:39443051 | A | G | rs79213093 | A | G |
| rs1046672447 | A | G | 15:39443068 | A | C | 15:39625546 | A | G |
| 15:39246929 | T | C | 15:39443137 | A | G | rs371827538 | T | C |
| 15:39247244 | T | C | 15:39443343 | A | T | 15:39625778 | A | G |
| 15:39247504 | C | G | 15:39443563 | A | T | rs1441293303 | A | G |
| 15:39247605 | A | C | 15:39444113 | T | C | rs1471318897 | T | C |
| 15:39248115 | A | G | 15:39444653 | T | C | 15:39627146 | C | G |
| 15:39248406 | A | G | 15:39444679 | T | G | 15:39627294 | T | C |
| 15:39248441 | T | C | 15:39444966 | A | G | 15:39627330 | A | C |
| 15:39248798 | A | T | 15:39445114 | T | C | rs1475514690 | A | G |
| rs1396264424 | A | G | 15:39445333 | T | C | 15:39627809 | A | G |
| 15:39248959 | T | C | 15:39445407 | T | C | 15:39627855 | T | C |
| 15:39249755 | A | G | rs1356324275 | A | G | 15:39627866 | A | G |
| rs545306682 | A | G | 15:39446251 | T | C | 15:39627982 | A | C |
| 15:39249829 | A | C | 15:39446820 | A | C | 15:39628131 | A | G |
| 15:39249984 | T | C | 15:39447205 | A | G | 15:39628187 | T | C |
| 15:39250088 | A | T | 15:39447529 | A | G | 15:39628260 | A | G |
| 15:39250097 | C | G | 15:39448180 | T | C | 15:39628287 | T | C |
| 15:39250128 | T | G | 15:39448288 | T | C | 15:39628309 | A | G |
| 15:39250129 | C | G | rs1441636957 | A | C | 15:39628403 | A | G |
| 15:39250617 | A | G | 15:39449003 | T | C | 15:39628562 | T | C |
| 15:39250642 | A | G | 15:39449131 | T | C | rs1026890735 | A | G |
| 15:39251190 | T | C | rs920796380 | A | G | 15:39628675 | T | C |
| 15:39251462 | T | G | rs944776293 | T | C | rs753667371 | T | C |
| rs900329231 | C | G | 15:39449489 | A | T | 15:39628866 | A | G |
| 15:39252031 | A | G | rs1019537285 | T | C | 15:39628909 | T | C |
| 15:39253829 | A | T | rs1261566281 | A | G | 15:39629050 | A | G |
| 15:39253904 | T | C | 15:39450612 | T | G | rs1014841409 | T | C |
| rs1426959587 | A | G | 15:39451170 | A | G | 15:39629777 | T | C |
| 15:39254837 | T | C | 15:39451210 | A | G | rs771565291 | A | G |
| 15:39254995 | A | G | 15:39452310 | A | T | 15:39630017 | T | C |
| 15:39255335 | T | C | 15:39453781 | T | G | 15:39630271 | T | C |
| rs549545866 | A | G | 15:39453970 | T | G | 15:39630461 | T | C |
| rs1487174277 | A | G | rs1220694991 | T | C | 15:39631119 | A | G |
| rs1238112816 | T | C | 15:39454860 | T | C | rs1028699112 | A | G |
| 15:39256931 | C | G | 15:39456466 | T | C | 15:39631771 | T | C |
| 15:39256937 | A | T | 15:39456631 | A | G | 15:39631967 | A | G |
| rs1473012905 | A | C | rs942698692 | A | T | rs1485370910 | C | G |
| rs895297945 | T | C | 15:39458292 | A | C | 15:39632269 | A | G |
| 15:39257870 | T | C | rs1385728447 | T | C | 15:39632490 | T | C |
| 15:39258832 | A | G | 15:39458912 | T | C | 15:39633765 | A | G |
| 15:39258866 | T | C | rs945978332 | A | G | 15:39633904 | A | G |
| 15:39258870 | A | C | 15:39459141 | T | C | 15:39633954 | T | C |
| 15:39259415 | A | C | 15:39459355 | A | G | 15:39634143 | T | C |
| 15:39259418 | A | G | rs935749871 | A | G | rs1242286056 | A | G |
| 15:39259504 | A | G | 15:39459369 | T | G | 15:39634303 | T | C |
| rs1045725844 | T | C | 15:39459475 | T | G | 15:39635124 | T | C |
| rs548367332 | T | C | 15:39459715 | T | C | 15:39636027 | C | G |
| 15:39260520 | A | C | 15:39460138 | T | C | 15:39636227 | T | C |
| rs1461798222 | A | T | 15:39462059 | T | C | 15:39636490 | C | G |
| 15:39260835 | T | C | 15:39462374 | A | G | 15:39636877 | T | C |
| 15:39260841 | C | G | rs1047239009 | A | T | 15:39636898 | T | C |
| rs1190970790 | A | T | 15:39462617 | A | C | 15:39637230 | C | G |
| 15:39261036 | A | C | 15:39463314 | A | G | rs1406044950 | T | C |
| 15:39261480 | A | G | rs559570153 | T | C | 15:39637529 | C | G |
| rs1002915096 | A | T | 15:39463691 | C | G | 15:39638023 | T | C |
| rs1369027062 | T | C | rs1415088054 | C | G | 15:39638395 | C | G |
| rs1294091560 | A | G | 15:39464115 | A | G | 15:39638543 | T | C |
| 15:39262124 | T | C | 15:39464167 | A | G | 15:39638686 | T | C |
| 15:39262165 | A | G | 15:39464752 | A | C | 15:39638932 | C | G |
| 15:39262265 | A | G | 15:39465344 | T | G | 15:39638991 | C | G |
| rs1360991351 | A | G | rs535677213 | T | C | 15:39639144 | A | G |
| 15:39262872 | A | T | 15:39465776 | A | T | 15:39639171 | C | G |
| rs1479341053 | A | C | 15:39466005 | A | G | 15:39639203 | T | C |
| 15:39263036 | A | G | 15:39466290 | A | C | 15:39639228 | A | G |
| 15:39263049 | C | G | 15:39466338 | T | C | 15:39639326 | A | G |
| 15:39263118 | A | G | rs1158719472 | T | C | 15:39639441 | A | G |
| 15:39263372 | A | G | rs1329100729 | A | G | 15:39639633 | A | T |
| rs1432543071 | A | G | 15:39466797 | A | G | 15:39639898 | A | C |
| 15:39264016 | C | G | 15:39466953 | A | C | 15:39639992 | A | G |
| 15:39264050 | A | G | 15:39466971 | C | G | 15:39640148 | T | C |
| 15:39264667 | T | G | 15:39467276 | A | G | 15:39640301 | T | C |
| 15:39264809 | T | C | 15:39467420 | T | C | rs1162496527 | T | C |
| rs1036706666 | T | C | 15:39467433 | T | C | 15:39640710 | T | C |
| 15:39265671 | A | G | 15:39467471 | T | C | 15:39640895 | A | G |
| 15:39265778 | T | C | 15:39467552 | A | G | 15:39641058 | A | G |
| 15:39265821 | T | C | 15:39467710 | A | G | 15:39641243 | A | C |
| rs534984276 | A | G | 15:39467770 | T | C | 15:39641338 | T | C |
| 15:39265911 | A | G | rs12148631 | A | G | 15:39641424 | A | G |
| 15:39266009 | T | C | 15:39468469 | T | C | rs1459558585 | A | C |
| 15:39266056 | A | G | 15:39468831 | C | G | 15:39641885 | A | G |
| rs1018330304 | T | C | 15:39469731 | A | G | 15:39641958 | A | G |
| 15:39266308 | A | G | 15:39470305 | C | G | 15:39642478 | A | G |
| 15:39266725 | A | G | rs982844036 | T | C | rs992893645 | A | G |
| 15:39266793 | C | G | 15:39470729 | T | C | 15:39642932 | T | C |
| rs956697344 | A | C | rs989592876 | A | G | 15:39643092 | A | G |
| 15:39267017 | A | T | 15:39471385 | A | G | 15:39643168 | T | C |
| 15:39267908 | C | G | rs901570387 | T | C | 15:39643175 | A | G |
| 15:39267951 | C | G | 15:39472628 | A | G | 15:39643601 | A | C |
| 15:39268912 | A | T | 15:39472665 | T | C | rs1334765234 | A | G |
| 15:39269390 | A | T | 15:39472683 | T | C | 15:39643695 | A | G |
| rs1252966008 | C | G | 15:39473093 | A | T | 15:39644427 | T | G |
| 15:39270049 | A | C | rs1380612364 | T | C | rs544300893 | A | G |
| 15:39270193 | C | G | 15:39474607 | A | G | 15:39646223 | A | G |
| rs1425438630 | T | C | 15:39474692 | T | C | rs1321329054 | T | C |
| 15:39270328 | C | G | 15:39475129 | C | G | 15:39647278 | A | G |
| 15:39270515 | T | C | 15:39476400 | A | C | 15:39647670 | T | G |
| 15:39270627 | T | G | rs780720034 | T | G | 15:39647797 | C | G |
| rs1007280681 | T | C | 15:39476805 | A | G | 15:39647894 | T | C |
| 15:39270787 | C | G | 15:39476963 | C | G | rs1187530859 | T | C |
| rs1199259658 | T | C | 15:39477137 | A | G | 15:39648251 | C | G |
| 15:39271165 | T | C | 15:39478241 | A | G | rs1255400543 | T | C |
| 15:39271265 | A | G | 15:39478409 | A | G | 15:39649306 | T | G |
| 15:39271293 | A | C | rs60451170 | T | C | 15:39649509 | T | C |
| 15:39271340 | A | G | 15:39479116 | A | G | 15:39650572 | C | G |
| 15:39271677 | T | C | 15:39479601 | T | C | 15:39651068 | T | C |
| rs542374249 | A | G | rs930580966 | A | G | 15:39651363 | T | C |
| rs1334167299 | T | C | 15:39481950 | T | C | rs925475010 | T | C |
| 15:39272379 | A | C | 15:39482084 | A | G | 15:39651534 | A | G |
| 15:39272457 | T | C | 15:39482451 | A | C | 15:39651637 | T | C |
| 15:39272505 | T | C | rs1164685848 | T | C | 15:39651750 | A | G |
| 15:39272564 | T | C | rs1169728883 | A | T | 15:39651875 | T | C |
| rs1382904434 | T | C | 15:39484756 | A | G | 15:39651902 | A | C |
| 15:39273534 | A | T | rs895383453 | A | C | 15:39652238 | T | C |
| 15:39273575 | A | C | 15:39485745 | T | C | 15:39652308 | A | G |
| rs1241565616 | A | C | 15:39485834 | T | C | 15:39652913 | C | G |
| rs1486750879 | A | G | 15:39485982 | A | G | 15:39653053 | A | C |
| 15:39273700 | T | C | 15:39486935 | A | G | 15:39653458 | T | C |
| 15:39273944 | C | G | 15:39486972 | A | T | 15:39654281 | A | G |
| 15:39274261 | A | C | rs1019735993 | A | G | 15:39654553 | A | G |
| 15:39274290 | A | G | 15:39487255 | A | G | 15:39654646 | A | T |
| 15:39274310 | A | T | rs1200973939 | A | C | 15:39654956 | A | G |
| 15:39274460 | A | C | rs192676943 | A | G | 15:39655356 | A | C |
| 15:39274478 | A | T | 15:39487697 | A | G | rs1172834822 | T | G |
| rs1037448707 | T | C | 15:39487972 | T | C | 15:39655622 | A | G |
| 15:39274609 | A | G | 15:39488869 | T | C | rs1160052381 | T | C |
| 15:39274670 | T | C | rs1037730650 | A | G | 15:39655924 | T | C |
| 15:39274817 | T | G | rs1353450878 | T | C | rs2411310 | C | G |
| rs1373021035 | A | T | rs1447868764 | A | G | 15:39656115 | A | G |
| 15:39274998 | A | G | rs1484560325 | A | T | 15:39656166 | T | C |
| 15:39275095 | A | G | 15:39491492 | C | G | 15:39656222 | A | C |
| 15:39275222 | A | T | 15:39491953 | A | G | 15:39657177 | A | G |
| 15:39275321 | T | C | 15:39491999 | T | C | 15:39657238 | T | C |
| 15:39275963 | A | G | 15:39492047 | A | G | 15:39657464 | A | G |
| 15:39275998 | T | C | 15:39492099 | C | G | 15:39657600 | A | G |
| rs998465654 | A | G | 15:39492267 | T | C | 15:39657646 | A | G |
| 15:39276318 | A | G | 15:39492309 | A | G | 15:39658191 | C | G |
| 15:39276701 | A | G | 15:39492426 | T | C | 15:39658443 | T | G |
| 15:39276949 | T | C | 15:39492776 | T | C | 15:39658985 | A | G |
| 15:39277034 | A | C | 15:39492839 | A | C | 15:39659178 | C | G |
| 15:39277124 | A | G | 15:39493179 | T | C | 15:39659184 | A | G |
| 15:39277140 | A | C | 15:39493463 | A | G | 15:39659211 | T | G |
| rs1336748419 | A | G | 15:39493465 | T | C | rs996101146 | A | G |
| 15:39277379 | A | G | rs1244630776 | T | C | 15:39661115 | A | C |
| 15:39277388 | C | G | 15:39493654 | A | G | 15:39661138 | A | G |
| 15:39277508 | T | C | rs1031879402 | A | T | rs903112394 | T | C |
| rs533328730 | C | G | 15:39494423 | A | G | rs1392205607 | A | T |
| 15:39277781 | T | C | 15:39494447 | T | G | 15:39662277 | A | G |
| 15:39278107 | A | C | rs1348027535 | A | T | rs1176824251 | A | T |
| 15:39278261 | T | C | 15:39494976 | A | C | 15:39662669 | T | G |
| 15:39278262 | A | G | rs1233706423 | T | C | 15:39662789 | A | C |
| rs1470432101 | T | C | 15:39495992 | T | C | 15:39662900 | T | C |
| 15:39278502 | T | C | rs1459714291 | T | C | rs1381359926 | A | G |
| rs4586401 | A | G | 15:39496525 | C | G | 15:39663756 | A | G |
| 15:39278940 | A | C | 15:39496544 | T | C | rs999577839 | A | C |
| 15:39279441 | T | C | 15:39496842 | A | T | rs1420334975 | C | G |
| 15:39279903 | A | G | 15:39497209 | A | T | 15:39664210 | A | G |
| 15:39280103 | A | G | 15:39497414 | A | G | rs912291277 | T | C |
| 15:39280655 | A | C | 15:39497486 | T | C | 15:39664956 | A | G |
| 15:39280797 | T | G | rs1013581913 | A | C | 15:39665465 | T | C |
| 15:39281191 | A | G | 15:39497651 | T | C | 15:39665499 | C | G |
| 15:39281432 | T | C | rs569776733 | A | G | 15:39665537 | A | G |
| 15:39281721 | A | G | 15:39498153 | A | G | rs1352893134 | T | G |
| 15:39281770 | A | C | 15:39498481 | A | T | 15:39665941 | T | C |
| 15:39281968 | T | C | 15:39498642 | A | C | 15:39666002 | A | G |
| 15:39282454 | T | C | 15:39498782 | A | G | 15:39666480 | T | C |
| 15:39282481 | T | C | rs749948972 | C | G | 15:39666960 | A | C |
| 15:39282648 | C | G | rs140195366 | A | T | 15:39667199 | A | G |
| 15:39282766 | C | G | 15:39499448 | A | G | 15:39667201 | A | G |
| 15:39282979 | A | T | 15:39499972 | A | G | 15:39667219 | T | C |
| 15:39282988 | A | G | 15:39500949 | T | C | 15:39667281 | T | G |
| 15:39283101 | T | C | 15:39500981 | C | G | 15:39667296 | T | C |
| 15:39283152 | A | G | rs1330940476 | A | T | 15:39667362 | A | G |
| 15:39283736 | A | T | rs116825171 | A | G | 15:39667882 | T | G |
| 15:39283767 | T | C | 15:39502388 | T | C | 15:39667921 | A | G |
| 15:39283979 | T | C | 15:39503050 | T | G | 15:39668328 | T | C |
| 15:39284445 | A | G | 15:39503192 | T | C | 15:39668329 | A | G |
| 15:39284513 | A | C | 15:39503508 | T | G | rs1031978318 | A | G |
| rs770952967 | A | T | rs1030801202 | T | C | 15:39668713 | A | G |
| 15:39284763 | A | G | 15:39504145 | A | G | 15:39668760 | A | G |
| 15:39285085 | A | C | 15:39504179 | T | G | rs556658945 | A | G |
| 15:39285601 | T | C | 15:39504194 | T | G | 15:39668870 | T | C |
| 15:39285635 | A | C | 15:39504512 | T | G | rs1440263733 | A | G |
| 15:39285679 | T | G | 15:39504624 | T | C | 15:39669952 | A | G |
| 15:39286538 | T | G | 15:39505007 | A | G | 15:39670175 | A | C |
| 15:39286543 | T | C | 15:39505716 | T | C | 15:39670370 | T | G |
| 15:39286628 | T | C | 15:39505815 | A | G | rs1405234474 | T | G |
| 15:39286732 | T | G | 15:39506341 | T | C | 15:39671113 | T | C |
| 15:39286859 | T | G | 15:39506360 | A | C | 15:39671188 | C | G |
| 15:39286919 | T | C | 15:39506788 | T | C | 15:39671514 | T | C |
| rs780391829 | A | G | 15:39506899 | A | C | 15:39671752 | T | G |
| 15:39286986 | T | C | 15:39507046 | T | C | 15:39671879 | A | G |
| 15:39287164 | A | G | 15:39507763 | T | C | 15:39672087 | A | C |
| 15:39287286 | T | C | 15:39507844 | A | G | 15:39672185 | A | G |
| rs892473175 | T | C | 15:39507940 | A | G | 15:39672231 | A | T |
| 15:39287452 | A | T | 15:39508124 | A | G | rs1271834367 | T | C |
| 15:39287694 | T | C | 15:39508271 | A | G | 15:39673762 | A | C |
| 15:39287953 | T | C | 15:39508745 | A | G | rs1024838512 | T | G |
| 15:39288125 | A | C | 15:39508822 | T | C | 15:39674385 | A | C |
| 15:39288527 | C | G | rs1448503109 | T | C | 15:39674839 | A | C |
| rs1377882160 | A | C | 15:39509914 | T | C | rs1285397052 | C | G |
| 15:39289017 | T | C | 15:39510030 | A | C | 15:39675440 | A | G |
| 15:39289029 | A | G | 15:39510216 | A | C | 15:39677152 | A | G |
| rs978718614 | T | C | rs879291665 | C | G | 15:39677303 | T | G |
| 15:39289415 | T | C | rs982363748 | A | G | 15:39677726 | T | C |
| 15:39289708 | T | G | 15:39511680 | A | T | rs918577994 | T | C |
| 15:39289834 | T | C | 15:39511698 | A | T | 15:39679805 | T | C |
| 15:39290352 | T | C | 15:39511759 | T | C | 15:39679996 | A | G |
| 15:39290682 | C | G | 15:39512099 | T | C | 15:39680795 | A | G |
| 15:39290741 | C | G | 15:39512301 | T | C | 15:39680810 | A | G |
| 15:39291535 | T | C | 15:39513190 | T | C | 15:39681236 | T | C |
| 15:39291996 | T | G | 15:39514615 | A | G | 15:39681337 | T | C |
| rs1305729721 | C | G | rs1398306890 | A | G | 15:39681857 | T | C |
| 15:39292434 | T | C | 15:39515693 | A | G | rs928175285 | A | G |
| 15:39292501 | A | G | 15:39516153 | C | G | 15:39682798 | A | C |
| rs1329025168 | A | C | 15:39516210 | T | G | 15:39683011 | T | G |
| 15:39292724 | A | G | 15:39516214 | A | G | 15:39683024 | A | T |
| 15:39293297 | A | T | 15:39516742 | A | G | 15:39683677 | T | C |
| 15:39293567 | T | C | 15:39517255 | A | C | 15:39685230 | A | G |
| 15:39293679 | T | C | 15:39518016 | T | C | rs186754460 | A | C |
| 15:39296956 | T | C | 15:39518040 | T | C | 15:39687127 | A | G |
| 15:39297872 | A | G | 15:39518244 | A | G | 15:39687402 | A | C |
| 15:39299494 | T | C | rs1475690227 | A | C | 15:39687451 | T | G |
| 15:39300266 | A | G | 15:39518563 | T | C | 15:39687926 | A | G |
| rs1256675948 | T | C | 15:39519092 | A | C | rs928056113 | T | C |
| 15:39300917 | A | T | 15:39519866 | A | G | 15:39690086 | A | G |
| 15:39301246 | A | G | rs530857032 | A | G | 15:39690109 | T | C |
| 15:39301285 | A | G | 15:39520759 | T | C | 15:39690384 | A | G |
| 15:39301668 | T | G | 15:39520813 | A | G | 15:39691319 | T | C |
| 15:39302638 | T | G | rs1482010418 | T | C | 15:39692647 | T | C |
| rs917179817 | A | C | rs1050618144 | A | G | 15:39693241 | A | G |
| 15:39303070 | T | C | 15:39520912 | T | C | 15:39693779 | A | G |
| 15:39303200 | A | G | 15:39521402 | A | G | 15:39694069 | A | G |
| 15:39303212 | C | G | 15:39521408 | A | G | 15:39694259 | A | G |
| rs1287682490 | A | G | 15:39521446 | T | G | 15:39694355 | A | C |
| rs1191994847 | A | T | 15:39521489 | A | G | 15:39694652 | A | G |
| rs1290786038 | T | C | 15:39521720 | A | G | 15:39694872 | A | G |
| 15:39304580 | A | C | rs552187265 | A | G | rs935344243 | A | C |
| 15:39305277 | C | G | 15:39522211 | C | G | 15:39695057 | A | G |
| 15:39305287 | T | C | 15:39522961 | A | G | 15:39695451 | A | T |
| 15:39305346 | A | G | rs1269708584 | C | G | 15:39696003 | A | G |
| 15:39305379 | C | G | 15:39523790 | A | G | 15:39696151 | C | G |
| 15:39305443 | T | C | 15:39524231 | C | G | 15:39697670 | C | G |
| 15:39305533 | A | G | 15:39524396 | A | G | 15:39698737 | A | G |
| 15:39305687 | C | G | 15:39524741 | T | C | 15:39698876 | A | G |
| 15:39305768 | A | G | rs527375099 | A | G | 15:39699662 | T | C |
| 15:39305804 | T | C | 15:39524869 | T | C | rs1344504431 | T | C |
| 15:39305854 | A | G | rs981753559 | T | C | 15:39700391 | T | C |
| 15:39305877 | A | C | 15:39525341 | T | C | 15:39701857 | T | C |
| rs1002907296 | A | G | 15:39525706 | T | G | 15:39702023 | T | G |
| 15:39306244 | C | G | 15:39525976 | C | G | 15:39702040 | A | T |
| rs562005242 | A | G | rs894241790 | T | C | 15:39702071 | C | G |
| 15:39306454 | T | C | 15:39526180 | C | G | 15:39702684 | A | C |
| 15:39306470 | T | G | 15:39526683 | A | C | 15:39703259 | T | C |
| 15:39306506 | T | C | 15:39527533 | T | C | 15:39703486 | C | G |
| 15:39306612 | A | C | 15:39527590 | A | G | 15:39703495 | T | C |
| 15:39306833 | A | G | 15:39527769 | C | G | 15:39703574 | A | G |
| 15:39306866 | A | G | 15:39527948 | T | C | 15:39703982 | T | C |
| 15:39307177 | A | G | rs1271058178 | A | C | 15:39704124 | T | C |
| rs1430780855 | T | G | 15:39528166 | A | C | rs1312754293 | A | T |
| rs1409843036 | T | C | 15:39528313 | T | C | 15:39704708 | T | C |
| 15:39308721 | A | T | 15:39528314 | A | G | 15:39704875 | T | C |
| 15:39308868 | A | G | 15:39528326 | T | C | 15:39704921 | T | G |
| 15:39309082 | A | C | rs1226685489 | A | G | 15:39704973 | A | T |
| 15:39309366 | A | T | rs1459361066 | T | C | rs954713288 | T | C |
| rs1325541352 | T | C | 15:39529285 | T | C | rs1200093092 | A | G |
| 15:39309558 | C | G | 15:39529325 | C | G | 15:39705721 | A | G |
| rs1319039751 | C | G | 15:39529717 | A | G | 15:39705961 | A | C |
| rs1056014243 | T | G | 15:39529985 | A | C | rs1054358711 | C | G |
| 15:39309815 | T | C | 15:39530108 | T | C | 15:39706673 | A | T |
| rs138253710 | T | C | rs943941497 | T | C | 15:39706723 | T | G |
| 15:39310585 | A | G | rs1270802535 | A | G | 15:39707006 | A | G |
| 15:39311059 | T | C | 15:39530508 | A | G | 15:39707453 | A | G |
| 15:39311249 | C | G | 15:39530983 | T | C | 15:39707516 | T | C |
| 15:39311550 | T | C | rs866097803 | T | C | 15:39707639 | A | G |
| 15:39311629 | A | G | 15:39531321 | A | G | 15:39707812 | A | G |
| 15:39311853 | A | G | 15:39531553 | C | G | rs1286239718 | T | C |
| 15:39312569 | A | G | 15:39531658 | A | G | 15:39708645 | A | G |
| 15:39312590 | T | C | 15:39531740 | T | C | 15:39708779 | T | G |
| 15:39312664 | A | T | 15:39532450 | A | G | 15:39708896 | T | C |
| 15:39313182 | A | C | 15:39532647 | A | C | 15:39709702 | T | C |
| rs1383564109 | C | G | 15:39532831 | T | C | 15:39709954 | C | G |
| 15:39314154 | A | T | rs1241764979 | T | C | 15:39709977 | T | C |
| 15:39314231 | A | T | 15:39533338 | A | G | 15:39710000 | C | G |
| 15:39315358 | C | G | rs1343229400 | A | G | 15:39710010 | A | G |
| 15:39315489 | C | G | 15:39533786 | T | G | rs1387227904 | T | C |
| 15:39315814 | C | G | 15:39534404 | A | C | 15:39710546 | A | C |
| 15:39315897 | A | G | rs1249642056 | A | G | 15:39710654 | A | G |
| 15:39316086 | A | G | rs751473011 | T | C | 15:39711188 | A | C |
| 15:39316187 | T | C | 15:39535900 | A | G | 15:39711353 | T | C |
| 15:39316630 | A | G | rs923731110 | T | C | 15:39711499 | A | G |
| 15:39317485 | C | G | rs1365646846 | T | C | 15:39712286 | T | C |
| 15:39318037 | T | C | 15:39537873 | T | C | 15:39719282 | T | G |
| | | | | | | rs548930095 | A | G |

TABLE 60
IPF transplant free survival variants associated with PUFIN3 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| 15: 39163935 | T | C | 15: 39166735 | A | G | 15: 39170014 | T | C |
| 15: 39164045 | T | C | 15: 39166885 | T | C | rs977256273 | T | C |
| 15: 39164780 | A | G | 15: 39167383 | A | G | rs1261621131 | A | G |
| 15: 39164809 | A | G | rs368280268 | A | G | 15: 39170985 | T | C |
| 15: 39164875 | A | G | 15: 39167916 | A | G | 15: 39171059 | A | G |
| 15: 39164921 | A | C | rs971179827 | A | G | 15: 39171614 | A | T |
| 15: 39164983 | T | C | 15: 39169241 | T | G | 15: 39171742 | T | G |
| 15: 39165053 | A | G | 15: 39169695 | A | G | 15: 39171746 | T | G |
| 15: 39165415 | C | G | 15: 39169698 | C | G | 15: 39171762 | A | T |
| 15: 39165581 | T | C | 15: 39169750 | A | C | 15: 39171851 | T | C |
| 15: 39165937 | T | C | 15: 39169779 | A | G | 15: 39171857 | T | C |
| 15: 39166073 | T | C | 15: 39169786 | A | C | 15: 39172049 | A | G |
| 15: 39166084 | A | C | 15: 39169885 | T | C | 15: 39172185 | A | G |
| 15: 39166417 | T | C | rs1439499168 | T | C | | | |
| rs1039139264 | C | G | 15: 39169960 | T | C | | | |

TABLE 61
IPF transplant free survival variants associated with PUFIN4 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| rs947816072 | T | C | 6: 169576847 | A | G | 6: 169583607 | T | C |
| 6: 169571488 | A | C | 6: 169577017 | A | G | 6: 169583681 | A | G |
| 6: 169571693 | T | C | 6: 169577210 | T | C | rs930175474 | A | G |
| 6: 169571815 | T | C | rs1413954450 | T | G | 6: 169584051 | A | G |
| 6: 169572144 | T | C | 6: 169577587 | T | C | 6: 169584184 | C | G |
| 6: 169572241 | A | G | rs900603489 | T | C | 6: 169584218 | A | G |
| 6: 169572248 | A | G | 6: 169577986 | A | G | 6: 169584264 | A | G |
| 6: 169572252 | T | C | 6: 169578348 | A | G | 6: 169584388 | T | C |
| rs1462581380 | A | G | 6: 169578362 | A | C | 6: 169584640 | A | G |
| 6: 169572487 | A | G | 6: 169578505 | T | C | 6: 169584647 | A | T |
| 6: 169572602 | A | G | 6: 169578555 | T | C | 6: 169584872 | C | G |
| 6: 169572709 | A | G | 6: 169578710 | A | G | 6: 169584891 | T | C |
| 6: 169572945 | A | T | 6: 169578944 | T | C | 6: 169585279 | A | C |
| 6: 169573006 | T | C | 6: 169579088 | T | C | 6: 169585616 | T | C |
| 6: 169573064 | A | G | 6: 169579194 | A | C | rs999404275 | A | G |
| 6: 169573184 | T | G | 6: 169579314 | A | G | 6: 169585679 | A | G |
| rs1383133712 | A | C | 6: 169579645 | T | C | rs964675529 | A | T |
| 6: 169573278 | A | G | 6: 169579816 | A | G | 6: 169585997 | A | G |
| 6: 169573396 | T | C | 6: 169580174 | A | G | rs1260729879 | T | G |
| 6: 169573400 | C | G | 6: 169580186 | T | C | rs1293921681 | A | T |
| 6: 169573835 | T | C | 6: 169580499 | T | C | 6: 169586825 | T | C |
| 6: 169573897 | T | C | rs1196476820 | C | G | rs1234821773 | T | C |
| 6: 169574046 | T | G | 6: 169581542 | T | C | rs1159164771 | A | G |
| 6: 169574144 | C | G | 6: 169581677 | T | G | 6: 169587148 | A | G |
| 6: 169574540 | A | G | 6: 169581833 | A | G | 6: 169587347 | A | G |
| rs909293721 | T | C | 6: 169582079 | A | G | 6: 169587711 | T | G |
| 6: 169574993 | T | C | 6: 169582411 | T | C | rs919177606 | T | G |
| rs112011691 | A | G | 6: 169582514 | A | G | 6: 169588023 | A | C |
| rs987612971 | C | G | 6: 169582569 | T | C | rs899907995 | A | G |
| 6: 169576138 | C | G | 6: 169582723 | T | C | 6: 169588279 | A | G |
| 6: 169576385 | T | C | 6: 169582985 | A | G | 6: 169588297 | C | G |
| 6: 169576552 | T | G | 6: 169583020 | T | G | 6: 169588360 | A | G |
| 6: 169576599 | C | G | 6: 169583214 | T | C | 6: 169588366 | T | C |
| 6: 169576765 | A | G | 6: 169583472 | T | G | | | |

TABLE 62
IPF transplant free survival variants associated with PUFIN5 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| rs563609128 | T | C | 4: 77694953 | A | T | 4: 77712281 | A | G |
| rs951701667 | T | C | 4: 77695021 | A | G | 4: 77712293 | A | G |
| rs1161784907 | T | C | rs1429751230 | T | C | 4: 77712584 | T | C |
| 4: 77682939 | T | G | 4: 77695128 | A | G | rs1376792774 | A | G |
| 4: 77684099 | A | G | 4: 77695161 | T | C | 4: 77713158 | A | C |
| 4: 77684512 | A | C | rs569666743 | T | C | rs1257179690 | A | C |
| 4: 77686695 | A | G | 4: 77695923 | T | C | 4: 77713790 | T | C |
| rs1161981172 | A | G | 4: 77696073 | A | G | 4: 77713874 | T | C |
| rs1026237227 | C | G | 4: 77696533 | T | C | 4: 77714410 | A | G |
| 4: 77687334 | T | C | rs1026582456 | A | G | rs1222298135 | T | C |
| 4: 77687993 | A | G | rs377486474 | T | C | 4: 77714534 | C | G |
| 4: 77688019 | C | G | 4: 77697071 | A | G | 4: 77714855 | A | G |
| 4: 77688152 | T | C | rs1455287857 | T | C | rs1224431218 | T | C |
| 4: 77688558 | A | G | 4: 77697928 | A | G | 4: 77716321 | C | G |
| 4: 77688592 | T | G | rs1289678495 | T | C | 4: 77716556 | T | G |
| 4: 77688688 | A | G | 4: 77701276 | A | T | 4: 77717083 | T | C |
| rs988026960 | A | G | 4: 77701312 | A | C | 4: 77717278 | C | G |
| 4: 77689717 | A | G | rs1359972917 | A | G | 4: 77717417 | T | C |
| 4: 77689855 | T | C | 4: 77702510 | A | G | 4: 77717762 | T | C |
| 4: 77690109 | A | G | 4: 77702689 | A | G | 4: 77717963 | A | C |
| 4: 77690224 | T | C | 4: 77703432 | T | G | 4: 77718377 | A | T |
| rs1295490228 | T | C | 4: 77704820 | T | C | 4: 77718392 | T | C |
| rs1345157131 | C | G | 4: 77706175 | A | G | 4: 77718456 | T | C |
| 4: 77691344 | T | C | 4: 77706234 | T | C | 4: 77718591 | T | G |
| rs1326578072 | A | G | 4: 77707008 | A | G | 4: 77718624 | A | G |
| 4: 77691764 | T | C | 4: 77707209 | T | C | 4: 77719340 | T | C |
| 4: 77692455 | T | C | 4: 77707376 | T | C | 4: 77719779 | A | G |
| 4: 77692534 | T | C | 4: 77707749 | A | G | rs562303709 | T | C |
| 4: 77692781 | A | G | rs1018337463 | A | T | 4: 77719935 | A | G |
| 4: 77693382 | A | G | 4: 77707780 | A | T | 4: 77720034 | C | G |
| 4: 77693429 | C | G | 4: 77707919 | T | C | 4: 77720135 | T | C |
| 4: 77693528 | A | G | 4: 77708325 | A | G | 4: 77720977 | C | G |
| 4: 77693636 | T | G | rs944017483 | T | C | 4: 77721086 | T | C |
| rs1199837466 | T | C | 4: 77709118 | A | C | 4: 77721605 | A | G |
| 4: 77693956 | C | G | 4: 77710383 | T | C | 4: 77721626 | C | G |
| 4: 77694232 | A | T | 4: 77710384 | A | T | 4: 77721651 | T | C |
| 4: 77694258 | A | G | 4: 77710427 | A | G | rs1399958760 | A | C |
| 4: 77694300 | C | G | 4: 77710893 | A | G | 4: 77721904 | A | G |
| 4: 77694385 | A | G | 4: 77711284 | T | C | 4: 77721997 | A | G |
| 4: 77694475 | T | C | 4: 77711313 | T | C | 4: 77722906 | A | G |
| 4: 77694753 | T | C | 4: 77712011 | A | T | | | |

TABLE 63
IPF transplant free survival variants associated with PUFIN6 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| 17: 48277749 | A | C | rs955596024 | A | G | 17: 48288396 | A | G |
| rs1411876148 | C | G | 17: 48283828 | A | G | 17: 48289070 | A | C |
| 17: 48278359 | C | G | 17: 48283884 | T | C | 17: 48289778 | C | G |
| 17: 48278507 | T | C | 17: 48284126 | C | G | 17: 48289855 | T | C |
| 17: 48279035 | A | G | 17: 48284268 | T | C | 17: 48290061 | A | G |
| 17: 48279521 | A | G | 17: 48284426 | A | G | 17: 48290286 | T | C |
| 17: 48280066 | A | G | 17: 48284814 | T | C | 17: 48290293 | A | G |
| 17: 48280356 | C | G | 17: 48285181 | T | C | 17: 48290686 | T | C |
| 17: 48280394 | A | T | 17: 48285375 | A | G | 17: 48291281 | T | C |
| 17: 48280990 | A | C | 17: 48285769 | A | G | 17: 48291366 | T | G |
| 17: 48281028 | A | G | 17: 48285993 | C | G | 17: 48292128 | T | G |
| rs2938479 | A | G | rs1485800765 | T | C | 17: 48292725 | T | G |
| 17: 48281235 | T | C | 17: 48286263 | C | G | 17: 48292844 | T | C |
| 17: 48281332 | C | G | 17: 48287411 | T | G | 17: 48292986 | T | C |
| rs550295774 | A | G | rs765113803 | T | C | 17: 48293081 | A | G |
| rs939965065 | C | G | 17: 48287807 | A | G | 17: 48293097 | A | G |
| 17: 48281911 | T | C | rs933589807 | C | G | 17: 48293254 | T | C |
| rs992055036 | A | G | 17: 48288035 | T | G | | | |

TABLE 64
IPF transplant free survival variants associated with PUFIN7 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| rs375021795 | T | C | 2: 216584523 | T | C | 2: 216585187 | A | G |
| 2: 216583355 | T | C | rs1305581474 | T | G | 2: 216585196 | T | C |
| 2: 216583873 | C | G | 2: 216585015 | T | C | 2: 216585356 | T | C |
| 2: 216583999 | A | G | 2: 216585023 | T | C | | | |
| 2: 216584458 | T | C | 2: 216585037 | T | C | | | |

TABLE 65
IPF transplant free survival variants associated with PUFIN8 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| rs761902257 | T | C | 14: 75022192 | T | C | 14: 75025162 | C | G |
| 14: 75019157 | T | C | 14: 75022271 | T | G | 14: 75025175 | A | G |
| 14: 75019875 | T | C | rs550947852 | A | G | 14: 75025713 | T | C |
| 14: 75021162 | A | G | 14: 75023369 | T | G | 14: 75026097 | A | G |
| 14: 75021396 | A | T | 14: 75023630 | A | G | 14: 75026125 | T | G |
| 14: 75021464 | T | C | rs910773189 | A | G | rs1255064335 | A | G |
| rs1285738968 | C | G | rs1017683821 | T | C | rs1032304895 | A | G |
| 14: 75021783 | A | G | 14: 75024752 | T | C | | | |
| 14: 75021952 | A | G | rs1398051432 | T | C | | | |

TABLE 66
IPF transplant free survival variants associated with PUFIN9 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| 2: 189831353 | A | G | 2: 189837995 | T | C | rs1407649417 | A | G |
| rs966226727 | T | C | 2: 189838122 | T | C | 2: 189844061 | C | G |
| rs1236010572 | C | G | 2: 189838389 | A | G | rs1469268371 | A | G |
| 2: 189833911 | T | C | 2: 189838800 | A | G | 2: 189844304 | A | C |
| 2: 189833962 | T | C | 2: 189839015 | T | C | rs5743114 | A | G |
| rs1029065539 | T | C | 2: 189839136 | A | G | 2: 189845515 | A | G |
| 2: 189834437 | A | G | 2: 189839530 | T | C | 2: 189845793 | A | G |
| rs978382033 | C | G | 2: 189839645 | A | G | 2: 189846133 | C | G |
| 2: 189836302 | T | G | 2: 189840221 | A | G | 2: 189847300 | A | G |
| 2: 189836303 | A | G | 2: 189841261 | T | C | rs1287402006 | T | C |
| 2: 189836589 | T | C | 2: 189841569 | A | C | 2: 189848384 | A | T |
| rs537048212 | A | T | rs78800852 | A | G | 2: 189848468 | T | C |
| 2: 189837112 | A | G | 2: 189841753 | A | C | 2: 189848940 | A | G |
| 2: 189837212 | T | C | 2: 189841916 | T | C | 2: 189849330 | A | G |
| 2: 189837378 | A | T | 2: 189842822 | A | T | 2: 189849355 | T | C |
| 2: 189837782 | A | C | 2: 189843516 | A | G | | | |

TABLE 67
IPF transplant free survival variants associated with PUFIN10 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| 11: 1239788 | T | C | 11: 1240485 | A | G | 11: 1241221 | T | G |
| rs768130429 | T | C | 11: 1241005 | T | C | rs1341136813 | A | G |

TABLE 68
IPF transplant free survival variants associated with PUFIN11 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| 6: 75508321 | T | G | 6: 75551717 | A | G | 6: 75668199 | C | G |
| 6: 75508733 | A | G | 6: 75551936 | T | C | rs1364351333 | T | C |
| 6: 75508785 | A | G | 6: 75551950 | A | C | 6: 75670186 | T | C |
| rs1008786107 | A | G | rs1364201823 | C | G | 6: 75670414 | T | C |
| rs141525330 | A | T | 6: 75552172 | A | G | rs541128050 | T | C |
| rs1337464001 | T | G | 6: 75552738 | A | G | 6: 75671302 | A | G |
| 6: 75511082 | T | C | rs1320989640 | T | C | 6: 75671324 | A | G |
| rs1035441084 | A | G | rs1446647071 | A | G | 6: 75671816 | A | T |
| 6: 75511841 | T | G | 6: 75553294 | T | C | 6: 75671821 | T | C |
| 6: 75512153 | T | C | 6: 75553805 | T | C | 6: 75672038 | T | C |
| 6: 75512286 | A | G | 6: 75554203 | A | G | 6: 75673095 | A | G |
| rs1426929665 | A | C | 6: 75554276 | T | C | rs898318803 | T | C |
| 6: 75512357 | A | G | 6: 75554369 | T | C | rs1300499297 | A | G |
| 6: 75512392 | A | T | 6: 75554442 | T | C | rs1174534770 | T | G |
| 6: 75512962 | A | T | rs1278148945 | A | T | 6: 75674223 | T | C |
| rs548350808 | T | C | 6: 75555307 | T | G | 6: 75675026 | A | G |
| 6: 75513075 | T | C | 6: 75555512 | A | C | 6: 75675093 | T | C |
| rs1284664354 | A | G | 6: 75555980 | C | G | 6: 75675446 | A | G |
| 6: 75513330 | A | C | 6: 75556207 | T | C | rs1216680498 | T | C |
| 6: 75513580 | T | C | 6: 75556479 | A | T | 6: 75676056 | C | G |
| 6: 75513968 | A | G | 6: 75557123 | A | G | 6: 75676252 | A | G |
| 6: 75514140 | T | G | rs760566421 | A | C | 6: 75676320 | T | G |
| 6: 75514175 | C | G | 6: 75557131 | T | C | rs769255313 | A | T |
| 6: 75514669 | A | G | 6: 75557719 | T | G | 6: 75677474 | C | G |
| 6: 75515201 | A | G | 6: 75557820 | T | C | 6: 75677567 | T | C |
| 6: 75516678 | A | G | 6: 75557870 | A | G | 6: 75678018 | A | G |
| 6: 75516703 | T | C | 6: 75558195 | A | G | 6: 75679114 | A | G |
| 6: 75516726 | T | C | 6: 75558759 | A | G | 6: 75679133 | T | C |
| rs1336822558 | T | C | 6: 75558906 | T | C | 6: 75679425 | T | C |
| 6: 75516849 | T | G | 6: 75559038 | T | C | rs1438996237 | A | G |
| rs1037075928 | A | G | 6: 75559265 | T | C | 6: 75680571 | A | G |
| 6: 75517251 | A | G | 6: 75559326 | A | G | 6: 75680713 | T | C |
| 6: 75517725 | A | G | 6: 75559564 | T | C | 6: 75681252 | T | G |
| 6: 75517861 | A | G | 6: 75559956 | C | G | 6: 75681469 | T | C |
| 6: 75518052 | A | G | 6: 75560087 | T | C | 6: 75681538 | T | C |
| 6: 75518053 | T | C | rs1311981307 | A | G | 6: 75682069 | T | C |
| rs1344268230 | A | G | 6: 75560341 | T | C | rs1447388256 | T | C |
| 6: 75519637 | T | G | 6: 75560529 | T | C | 6: 75682797 | A | T |
| 6: 75520343 | C | G | 6: 75560616 | C | G | rs1258985183 | A | G |
| 6: 75520737 | T | C | 6: 75560968 | T | G | rs898167301 | A | G |
| 6: 75520837 | C | G | rs1354906482 | T | C | rs1215333874 | A | T |
| 6: 75520922 | T | C | 6: 75561056 | A | G | 6: 75685600 | A | G |
| 6: 75521046 | A | T | 6: 75561571 | T | G | 6: 75685871 | A | G |
| 6: 75521201 | T | C | 6: 75561591 | A | G | 6: 75685973 | A | T |
| 6: 75521514 | A | C | 6: 75561757 | A | T | rs1435295210 | T | C |
| rs1052101860 | T | C | rs774177244 | A | G | rs1266654342 | T | C |
| 6: 75521693 | A | G | 6: 75562039 | A | C | rs1355863126 | T | C |
| 6: 75521765 | A | G | 6: 75562196 | T | C | 6: 75687247 | A | G |
| 6: 75521897 | A | T | 6: 75562286 | T | G | 6: 75687997 | T | C |
| 6: 75522030 | T | C | 6: 75562885 | T | C | 6: 75688817 | A | G |
| 6: 75522110 | A | C | 6: 75563041 | A | T | 6: 75689747 | T | G |
| rs531921749 | T | G | 6: 75564033 | A | G | rs1439447447 | A | G |
| 6: 75522187 | A | G | 6: 75564396 | C | G | 6: 75690656 | A | T |
| 6: 75522348 | T | G | 6: 75567052 | A | C | rs1205016261 | A | C |
| 6: 75522351 | T | C | 6: 75567726 | T | C | 6: 75691451 | A | G |
| rs546843403 | A | G | 6: 75568360 | A | G | 6: 75691488 | T | G |
| rs909437240 | A | G | 6: 75569832 | T | C | 6: 75691885 | A | C |
| 6: 75522706 | A | G | 6: 75570292 | T | C | 6: 75692113 | A | C |
| 6: 75523058 | A | G | 6: 75570548 | T | C | 6: 75692413 | T | C |
| 6: 75523616 | A | G | 6: 75570727 | A | G | 6: 75694356 | C | G |
| rs116285756 | T | C | 6: 75571074 | T | G | 6: 75694758 | A | G |
| 6: 75523948 | T | C | 6: 75571713 | T | G | 6: 75694896 | A | G |
| 6: 75523976 | T | C | rs746445565 | T | C | 6: 75695517 | A | T |
| 6: 75524953 | T | C | 6: 75572277 | A | G | rs935742456 | A | G |
| 6: 75525178 | T | C | 6: 75572299 | T | C | 6: 75695962 | T | C |
| 6: 75525296 | T | G | rs889014630 | A | G | 6: 75696027 | T | C |
| rs1243675275 | A | G | 6: 75572443 | A | G | 6: 75696331 | C | G |
| 6: 75525548 | A | G | rs1044202583 | A | C | 6: 75696630 | T | C |
| rs1261020033 | T | G | 6: 75572814 | C | G | 6: 75697511 | T | C |
| 6: 75525739 | T | C | 6: 75573187 | T | C | 6: 75697531 | A | G |
| 6: 75525927 | A | T | 6: 75573867 | A | C | 6: 75697947 | A | G |
| rs1180470929 | C | G | 6: 75574046 | T | C | 6: 75698168 | A | C |
| 6: 75526165 | A | G | 6: 75574400 | A | G | 6: 75699299 | A | G |
| 6: 75526624 | A | C | 6: 75574836 | C | G | 6: 75699322 | T | C |
| 6: 75526838 | A | G | rs113020307 | T | G | 6: 75700158 | T | C |
| 6: 75526916 | A | G | 6: 75576040 | A | T | 6: 75700246 | A | C |
| 6: 75527052 | A | C | 6: 75576325 | T | C | 6: 75701127 | C | G |
| 6: 75527662 | A | G | 6: 75576427 | A | G | 6: 75701502 | C | G |
| 6: 75527687 | A | G | 6: 75577097 | T | G | 6: 75701667 | T | G |
| 6: 75527717 | A | G | 6: 75577359 | T | C | 6: 75702318 | A | G |
| 6: 75527856 | A | G | 6: 75577453 | T | C | 6: 75702692 | A | C |
| 6: 75528205 | A | G | 6: 75577861 | T | C | rs781619667 | A | G |
| 6: 75528647 | A | C | 6: 75578046 | T | C | 6: 75703522 | C | G |
| 6: 75528741 | T | G | 6: 75578119 | A | G | rs1464231261 | A | G |
| 6: 75528903 | A | G | 6: 75578149 | A | G | 6: 75705146 | T | C |
| 6: 75528959 | T | C | 6: 75579300 | A | G | 6: 75707099 | C | G |
| 6: 75529012 | A | G | 6: 75580548 | A | T | 6: 75707227 | A | G |
| 6: 75529074 | T | G | 6: 75581563 | A | G | 6: 75707632 | A | G |
| 6: 75529992 | T | G | rs940915976 | A | G | rs987810587 | A | G |
| 6: 75530228 | A | C | rs1437883169 | A | C | rs910582512 | A | C |
| 6: 75530315 | T | C | rs999533833 | A | G | 6: 75708289 | A | G |
| rs1016499867 | C | G | 6: 75584667 | T | C | rs1490002922 | A | C |
| 6: 75531172 | A | G | 6: 75584679 | A | G | 6: 75709170 | A | C |
| 6: 75531303 | T | C | 6: 75584893 | A | T | 6: 75711165 | T | G |
| 6: 75531414 | T | C | 6: 75584957 | T | C | 6: 75712122 | T | G |
| 6: 75531422 | A | G | 6: 75585492 | T | C | 6: 75712166 | A | T |
| 6: 75531666 | C | G | 6: 75585527 | T | C | 6: 75712490 | A | G |
| 6: 75531681 | T | C | rs905457240 | T | C | 6: 75713432 | A | G |
| rs1022311086 | A | G | 6: 75586618 | A | G | 6: 75713970 | T | C |
| 6: 75532250 | T | G | 6: 75587079 | T | C | 6: 75714093 | A | C |
| rs1321405263 | A | G | rs1415089898 | A | G | rs1485828008 | A | G |
| rs1253830077 | A | G | 6: 75588358 | A | C | 6: 75714149 | C | G |
| 6: 75532536 | T | G | 6: 75588524 | T | C | rs897548512 | T | C |
| 6: 75532577 | T | C | 6: 75590052 | A | G | 6: 75714270 | T | C |
| rs1162144116 | T | C | 6: 75590535 | T | C | 6: 75716121 | T | C |
| 6: 75533070 | A | T | 6: 75591021 | A | G | 6: 75716353 | T | C |
| 6: 75533126 | T | C | 6: 75592649 | A | G | 6: 75716729 | A | G |
| rs762289057 | T | C | 6: 75592787 | A | G | 6: 75717579 | T | C |
| rs1377659664 | T | C | 6: 75592989 | T | G | 6: 75718038 | A | G |
| 6: 75533420 | A | G | 6: 75593095 | A | C | 6: 75719030 | A | G |
| 6: 75533452 | T | C | 6: 75593540 | T | C | 6: 75720431 | A | G |
| 6: 75533702 | A | T | 6: 75593656 | A | G | rs189331100 | T | C |
| 6: 75533863 | A | C | rs1331884979 | T | C | 6: 75720952 | A | T |
| 6: 75533897 | A | G | 6: 75595964 | T | C | 6: 75721085 | A | T |
| 6: 75534047 | T | G | 6: 75596235 | A | G | 6: 75721569 | A | C |
| 6: 75534152 | T | C | 6: 75597332 | T | C | 6: 75722491 | A | G |
| 6: 75534228 | C | G | 6: 75597748 | T | G | rs894482977 | T | C |
| 6: 75534430 | A | C | 6: 75598042 | T | C | 6: 75722937 | A | C |
| 6: 75534536 | A | G | 6: 75598427 | A | G | 6: 75723079 | A | G |
| 6: 75534581 | T | C | 6: 75598481 | A | G | 6: 75723629 | T | C |
| 6: 75534950 | T | C | 6: 75598654 | T | C | 6: 75724241 | A | C |
| 6: 75534970 | A | G | 6: 75598983 | A | G | rs1281763607 | T | G |
| 6: 75535025 | A | G | 6: 75599121 | A | G | 6: 75725065 | A | T |
| 6: 75535080 | T | C | 6: 75599386 | A | G | 6: 75725654 | T | C |
| 6: 75535355 | C | G | 6: 75600016 | A | G | rs1013610191 | A | T |
| 6: 75535509 | A | C | 6: 75600201 | T | G | 6: 75727245 | T | C |
| 6: 75536221 | A | G | 6: 75600804 | T | C | 6: 75728009 | T | C |
| 6: 75536429 | A | G | 6: 75600908 | A | G | 6: 75728077 | A | G |
| rs1021292185 | T | C | 6: 75602656 | T | C | 6: 75728179 | T | C |
| 6: 75536669 | C | G | 6: 75602977 | C | G | rs941600582 | A | G |
| 6: 75536681 | A | G | 6: 75604845 | A | G | rs576067761 | T | C |
| 6: 75536823 | T | G | rs1484568603 | T | C | 6: 75732084 | A | G |
| 6: 75536917 | A | C | 6: 75605445 | T | G | rs1269689531 | A | G |
| 6: 75536940 | A | G | rs58570618 | A | G | 6: 75733775 | A | G |
| 6: 75537109 | A | G | 6: 75606397 | T | G | 6: 75734077 | C | G |
| 6: 75537122 | T | G | 6: 75606514 | T | C | 6: 75734973 | T | C |
| 6: 75537200 | A | C | rs1223188722 | A | G | 6: 75735643 | A | T |
| 6: 75537573 | T | G | 6: 75607294 | T | C | 6: 75737881 | T | C |
| 6: 75537926 | T | G | 6: 75608454 | C | G | 6: 75737968 | A | G |
| 6: 75537948 | A | G | 6: 75608690 | T | C | 6: 75739925 | A | G |
| 6: 75538210 | A | G | rs1302638274 | T | C | 6: 75740057 | A | C |
| 6: 75538225 | A | G | 6: 75610722 | T | G | 6: 75740176 | T | G |
| rs753366709 | T | C | 6: 75611637 | A | G | 6: 75740700 | T | C |
| 6: 75538379 | T | C | rs1264384851 | A | T | 6: 75741330 | T | C |
| 6: 75538524 | A | G | rs1341518925 | T | C | 6: 75741836 | A | G |
| 6: 75538575 | A | G | rs766499680 | T | C | 6: 75742044 | A | G |
| 6: 75538622 | A | G | 6: 75612601 | A | G | rs1165138247 | A | G |
| 6: 75538631 | T | C | 6: 75613111 | C | G | rs1440086130 | T | C |
| 6: 75538718 | T | G | rs1269479703 | A | G | 6: 75743533 | T | C |
| 6: 75538722 | A | G | rs1283180852 | A | G | 6: 75745366 | A | G |
| 6: 75538762 | A | T | 6: 75613822 | T | C | 6: 75745798 | T | C |
| rs758824251 | A | T | 6: 75613832 | C | G | 6: 75745875 | T | C |
| 6: 75538836 | T | C | 6: 75613869 | A | G | 6: 75745901 | T | C |
| 6: 75538940 | T | G | rs1346779412 | T | G | 6: 75746765 | A | T |
| 6: 75539046 | A | G | rs971907780 | A | G | 6: 75747378 | A | C |
| 6: 75539142 | A | G | 6: 75615589 | A | G | 6: 75749256 | A | G |
| 6: 75539208 | T | C | rs977270154 | C | G | 6: 75749392 | A | G |
| 6: 75539431 | A | G | 6: 75615973 | T | C | 6: 75750042 | C | G |
| 6: 75539434 | A | G | 6: 75616164 | T | C | 6: 75750133 | T | C |
| 6: 75539478 | T | C | 6: 75616948 | A | T | 6: 75751339 | T | C |
| 6: 75539539 | T | C | 6: 75617272 | A | T | 6: 75751429 | T | C |
| 6: 75539629 | T | C | 6: 75617743 | T | G | rs1050472068 | T | G |
| 6: 75539666 | T | C | 6: 75618555 | C | G | 6: 75752306 | C | G |
| 6: 75539717 | A | C | rs1266415821 | A | C | 6: 75752316 | T | C |
| rs1434239624 | T | C | 6: 75620220 | T | G | rs539691428 | A | G |
| 6: 75539866 | A | T | rs1042958293 | A | T | 6: 75753175 | T | C |
| 6: 75539914 | A | G | 6: 75620440 | A | G | 6: 75753342 | T | C |
| 6: 75540006 | A | T | 6: 75620466 | T | G | 6: 75753594 | T | C |
| 6: 75540174 | A | G | 6: 75620721 | A | G | 6: 75753595 | A | C |
| 6: 75540246 | C | G | rs1272215868 | A | G | 6: 75754020 | T | C |
| 6: 75540363 | T | G | 6: 75622440 | A | G | 6: 75754065 | A | G |
| 6: 75540435 | A | G | 6: 75622882 | A | G | 6: 75754877 | A | T |
| 6: 75540457 | T | C | 6: 75625251 | A | C | 6: 75754917 | T | G |
| 6: 75540478 | A | G | 6: 75625362 | T | C | 6: 75755218 | A | T |
| 6: 75540670 | A | G | 6: 75625903 | A | G | 6: 75755603 | A | G |
| 6: 75540870 | T | C | 6: 75625966 | T | C | 6: 75756117 | A | G |
| 6: 75540963 | A | G | 6: 75626058 | T | C | 6: 75757543 | T | C |
| 6: 75541238 | A | G | 6: 75627754 | A | G | 6: 75757561 | T | C |
| 6: 75541475 | A | G | 6: 75628412 | A | T | 6: 75757573 | T | C |
| 6: 75541577 | A | C | 6: 75630208 | A | G | 6: 75759911 | T | C |
| 6: 75541660 | A | T | 6: 75630423 | A | G | 6: 75761144 | T | C |
| rs889763492 | T | C | 6: 75630562 | A | G | rs1451802622 | T | G |
| 6: 75542009 | A | G | 6: 75630790 | T | C | 6: 75762044 | A | G |
| 6: 75542080 | T | C | 6: 75631168 | A | G | 6: 75762298 | T | C |
| 6: 75542459 | A | G | 6: 75632019 | A | G | 6: 75762882 | C | G |
| 6: 75542717 | A | G | 6: 75632257 | A | G | 6: 75763552 | A | G |
| rs769638811 | A | G | 6: 75632652 | C | G | 6: 75764992 | C | G |
| 6: 75542900 | A | G | 6: 75632693 | A | T | rs1158922273 | T | G |
| 6: 75542933 | T | C | 6: 75632997 | T | C | 6: 75765898 | T | C |
| 6: 75542980 | T | C | 6: 75634577 | A | G | 6: 75766515 | A | G |
| 6: 75543270 | A | G | 6: 75634822 | A | G | 6: 75766579 | A | C |
| 6: 75543546 | T | C | 6: 75634825 | T | C | rs1440059508 | A | C |
| 6: 75543827 | A | C | 6: 75635813 | A | G | rs71561441 | T | C |
| 6: 75543866 | A | G | 6: 75636574 | T | C | rs1250128289 | T | C |
| 6: 75543878 | T | C | rs762260971 | T | G | 6: 75768987 | T | C |
| 6: 75543964 | T | C | 6: 75637216 | A | G | rs1347006317 | T | C |
| 6: 75544095 | T | C | 6: 75637364 | T | C | 6: 75769110 | A | G |
| 6: 75544130 | A | G | rs902192468 | A | G | 6: 75769395 | T | C |
| 6: 75544607 | T | C | 6: 75638092 | C | G | rs1348249532 | T | C |
| 6: 75544801 | A | C | 6: 75638299 | T | G | 6: 75769615 | A | G |
| 6: 75544942 | T | C | 6: 75638514 | A | T | rs1478964549 | T | G |
| 6: 75545026 | A | G | 6: 75639575 | A | G | 6: 75770777 | T | C |
| rs1481473034 | A | G | rs868157139 | A | G | 6: 75771435 | A | G |
| 6: 75545338 | T | C | 6: 75639837 | T | C | rs1463870268 | A | C |
| rs1272445573 | A | T | 6: 75640192 | T | C | rs1297066640 | A | C |
| 6: 75545404 | T | C | 6: 75640276 | A | G | 6: 75775381 | T | G |
| 6: 75545503 | A | G | rs537422300 | A | G | 6: 75776345 | A | G |
| 6: 75545675 | A | G | rs549808895 | A | G | 6: 75776760 | T | C |
| 6: 75545720 | T | C | 6: 75641875 | T | C | 6: 75776984 | A | G |
| 6: 75545767 | A | G | 6: 75642282 | T | C | 6: 75777148 | T | C |
| rs1234157830 | A | C | 6: 75642415 | A | G | 6: 75777241 | T | G |
| 6: 75546153 | T | C | 6: 75642822 | A | G | 6: 75778665 | A | G |
| 6: 75547024 | A | C | 6: 75642984 | A | G | rs1177315840 | T | C |
| rs1192696532 | A | G | 6: 75643138 | A | G | 6: 75779343 | A | G |
| 6: 75547170 | T | G | 6: 75644222 | A | G | 6: 75779533 | T | C |
| 6: 75547183 | T | C | 6: 75645136 | T | C | 6: 75779564 | T | C |
| 6: 75547471 | A | G | 6: 75645260 | T | G | 6: 75779950 | A | G |
| rs1030088494 | T | C | 6: 75645453 | T | C | 6: 75780530 | A | G |
| 6: 75547944 | T | G | 6: 75645868 | A | G | 6: 75780564 | T | C |
| 6: 75547951 | A | G | rs1048112024 | T | C | 6: 75780764 | C | G |
| 6: 75548035 | T | C | 6: 75646760 | A | G | rs1242227336 | A | T |
| 6: 75548240 | A | C | 6: 75646852 | A | G | 6: 75782320 | A | G |
| 6: 75548272 | A | C | 6: 75647521 | A | G | 6: 75782622 | T | G |
| 6: 75548437 | T | C | 6: 75648453 | A | G | rs976151949 | A | G |
| 6: 75548503 | A | C | 6: 75648744 | T | C | rs1293162371 | T | C |
| 6: 75548534 | T | C | 6: 75649152 | T | C | 6: 75783991 | A | C |
| 6: 75548749 | A | G | rs894253468 | T | C | 6: 75785178 | T | C |
| rs1250579336 | T | C | rs965810211 | T | C | 6: 75785671 | T | C |
| rs1486505033 | T | C | 6: 75651807 | T | C | 6: 75789526 | T | C |
| 6: 75549552 | T | C | 6: 75652748 | A | C | rs1180908665 | A | G |
| 6: 75549672 | A | G | 6: 75652823 | T | C | 6: 75790389 | T | C |
| 6: 75549705 | T | C | 6: 75653435 | A | G | rs1349281866 | A | T |
| 6: 75549825 | T | G | 6: 75654467 | T | C | 6: 75792353 | A | T |
| 6: 75549830 | T | G | 6: 75656031 | A | C | 6: 75792756 | A | G |
| 6: 75549979 | A | G | 6: 75656786 | A | G | 6: 75792781 | T | C |
| 6: 75550018 | A | C | 6: 75656974 | A | C | 6: 75797302 | T | C |
| 6: 75550062 | T | C | 6: 75660345 | A | C | 6: 75797391 | T | C |
| 6: 75550101 | C | G | 6: 75661247 | A | C | 6: 75797549 | A | G |
| 6: 75550236 | A | G | 6: 75661566 | A | G | rs1367377497 | A | C |
| 6: 75550598 | A | G | 6: 75662836 | A | G | 6: 75797800 | T | C |
| 6: 75550599 | T | C | 6: 75663351 | A | C | 6: 75798429 | A | G |
| rs1457759725 | A | G | 6: 75663471 | T | C | 6: 75800793 | A | G |
| 6: 75550978 | A | T | 6: 75664105 | A | C | 6: 75800931 | T | C |
| rs987118458 | T | C | 6: 75664109 | A | G | rs202095922 | A | T |
| 6: 75551164 | T | C | 6: 75664956 | T | C | 6: 75802471 | T | C |
| 6: 75551342 | A | C | 6: 75665090 | T | G | 6: 75802507 | T | C |
| 6: 75551444 | T | C | 6: 75665399 | C | G | rs1006104757 | A | T |
| 6: 75551617 | T | C | 6: 75665409 | T | C | 6: 75804783 | A | G |
| 6: 75551626 | T | C | 6: 75666137 | T | G | | | |
| 6: 75551657 | A | C | rs936770897 | A | G | | | |
| rs1382138344 | T | C | 6: 75667061 | T | G | | | |

TABLE 69
IPF transplant free survival variants associated with PUFIN12 transcript.

| | | Non- | | | Non- | | | Non- |
|---|---|---|---|---|---|---|---|---|
| | Effect | effect | | Effect | effect | | Effect | effect |
| rsid | Allele | allele | rsid | Allele | allele | rsid | Allele | allele |
| 16: 86879172 | T | C | 16: 86962880 | A | G | 16: 87029323 | A | G |
| 16: 86879475 | T | C | 16: 86963005 | T | C | 16: 87029635 | A | T |
| 16: 86879830 | T | G | 16: 86963186 | A | G | 16: 87029769 | C | G |
| 16: 86879923 | A | T | 16: 86963373 | T | C | 16: 87029789 | C | G |
| 16: 86880376 | A | G | 16: 86963844 | C | G | rs1311434971 | A | G |
| 16: 86880674 | T | C | rs188506693 | C | G | 16: 87030179 | A | T |
| 16: 86881001 | T | C | 16: 86964036 | A | G | 16: 87030214 | T | C |
| 16: 86881362 | A | G | 16: 86964379 | A | C | 16: 87030219 | C | G |
| 16: 86882110 | A | G | 16: 86965024 | A | G | rs928658850 | A | G |
| 16: 86882139 | T | C | rs1490966927 | T | C | 16: 87030390 | A | G |
| 16: 86882168 | A | C | rs955400995 | C | G | 16: 87030561 | T | C |
| rs189507290 | T | C | rs145360433 | T | G | rs1021590381 | T | C |
| 16: 86882228 | A | G | 16: 86965989 | T | G | rs967478568 | T | G |
| 16: 86882251 | C | G | rs12446284 | C | G | rs1453064442 | A | T |
| 16: 86883263 | A | G | 16: 86966053 | T | C | 16: 87030805 | T | C |
| 16: 86883375 | A | G | rs188940506 | A | G | 16: 87030847 | A | G |
| rs74036654 | T | C | rs951049037 | A | G | 16: 87030956 | T | C |
| 16: 86884164 | A | C | 16: 86966485 | A | G | 16: 87031056 | A | C |
| rs1309735220 | A | G | rs1181958877 | T | C | 16: 87031062 | T | C |
| rs1421701585 | A | G | rs1008284441 | A | T | rs753486631 | T | C |
| 16: 86884333 | A | G | 16: 86966632 | A | G | rs1486540646 | A | G |
| 16: 86884575 | T | C | rs1370082398 | A | T | 16: 87031303 | T | C |
| 16: 86884651 | T | C | 16: 86966707 | A | G | 16: 87031304 | A | G |
| rs1451891491 | T | C | rs775333721 | T | C | 16: 87031309 | A | C |
| 16: 86885225 | C | G | 16: 86966971 | A | G | 16: 87031351 | A | G |
| rs1170269569 | A | C | 16: 86967051 | T | C | 16: 87031536 | A | G |
| rs1299900755 | A | G | 16: 86967234 | A | G | 16: 87031564 | A | G |
| 16: 86885518 | T | C | 16: 86967265 | T | C | rs1043760808 | T | C |
| 16: 86885542 | T | C | rs143640506 | A | G | rs1285389186 | T | C |
| 16: 86885593 | A | T | 16: 86968343 | A | G | rs997173219 | A | G |
| 16: 86886246 | T | G | 16: 86968447 | A | G | rs992101937 | T | C |
| 16: 86886543 | A | G | 16: 86968712 | A | G | rs1470452989 | C | G |
| 16: 86886604 | T | C | 16: 86968754 | A | G | 16: 87032294 | A | C |
| 16: 86886670 | T | C | 16: 86969210 | T | C | 16: 87032408 | T | G |
| 16: 86886714 | T | C | 16: 86969363 | A | T | 16: 87032527 | T | C |
| 16: 86887058 | T | C | 16: 86969486 | T | C | rs937261198 | C | G |
| 16: 86887388 | A | T | rs1206855941 | T | G | 16: 87032601 | T | C |
| 16: 86887448 | T | C | 16: 86969724 | A | G | 16: 87032647 | C | G |
| rs1320958354 | A | G | rs776929246 | A | G | rs12595901 | T | C |
| 16: 86887676 | A | G | 16: 86970212 | A | G | 16: 87033282 | T | C |
| 16: 86888065 | T | C | 16: 86970454 | A | G | 16: 87033405 | T | C |
| 16: 86888152 | A | G | 16: 86970479 | T | C | 16: 87033474 | C | G |
| rs1344385188 | A | G | 16: 86970503 | A | C | 16: 87033705 | T | G |
| 16: 86888574 | T | C | 16: 86970633 | T | C | 16: 87033714 | A | G |
| 16: 86888863 | T | G | rs547924968 | A | T | 16: 87034282 | A | T |
| 16: 86888942 | A | G | 16: 86970995 | A | G | rs1456035641 | T | C |
| 16: 86889249 | T | C | 16: 86971077 | T | C | rs1414102040 | A | T |
| 16: 86889560 | T | C | 16: 86971630 | T | C | rs1027052582 | T | C |
| 16: 86889621 | A | G | 16: 86971663 | A | C | 16: 87034712 | T | C |
| rs1223559192 | T | C | 16: 86972178 | A | G | 16: 87034722 | A | T |
| 16: 86890014 | T | C | 16: 86972183 | T | C | 16: 87034758 | T | G |
| 16: 86890468 | T | C | 16: 86972212 | A | G | 16: 87034828 | A | G |
| 16: 86890533 | C | G | 16: 86972555 | A | T | 16: 87034874 | T | C |
| 16: 86890700 | C | G | 16: 86972701 | T | C | 16: 87034885 | T | C |
| 16: 86890722 | T | C | 16: 86972876 | A | T | rs1021561876 | T | C |
| 16: 86890740 | T | C | 16: 86973196 | C | G | 16: 87035113 | T | G |
| 16: 86890840 | T | C | rs1260894289 | T | G | rs1426906220 | C | G |
| rs1388654066 | A | G | 16: 86973468 | A | G | 16: 87035241 | A | G |
| 16: 86891790 | C | G | rs1381688831 | A | G | rs937781960 | A | T |
| rs1015865281 | T | C | 16: 86974973 | A | T | rs1240094993 | T | C |
| 16: 86892017 | C | G | 16: 86975044 | A | G | 16: 87035586 | A | G |
| 16: 86892072 | T | C | 16: 86975155 | C | G | rs572802182 | C | G |
| 16: 86892464 | C | G | 16: 86975204 | C | G | 16: 87036156 | T | C |
| rs923483059 | A | G | rs75444601 | A | G | rs531575829 | T | C |
| 16: 86893902 | C | G | 16: 86976339 | A | C | 16: 87036486 | A | T |
| 16: 86894376 | A | G | 16: 86976433 | T | C | rs537505173 | T | C |
| 16: 86895761 | T | C | 16: 86977008 | A | C | 16: 87036843 | T | C |
| 16: 86895915 | C | G | rs1267392449 | T | C | 16: 87036947 | A | C |
| rs991479047 | T | C | 16: 86977214 | T | C | 16: 87037125 | T | C |
| 16: 86896105 | A | G | 16: 86977582 | T | C | 16: 87037206 | C | G |
| 16: 86896199 | A | T | 16: 86977962 | T | C | 16: 87037459 | T | C |
| 16: 86896530 | A | G | 16: 86978033 | A | T | 16: 87037608 | T | C |
| 16: 86896671 | T | G | rs920707792 | T | C | 16: 87037864 | C | G |
| rs1341472521 | T | C | 16: 86979422 | A | G | 16: 87038045 | T | C |
| 16: 86896940 | C | G | 16: 86979824 | T | C | 16: 87038209 | T | G |
| rs143418278 | A | G | 16: 86980044 | T | C | rs915898613 | A | G |
| 16: 86897796 | T | G | 16: 86980112 | T | C | 16: 87038679 | T | C |
| 16: 86897823 | T | G | 16: 86980149 | C | G | 16: 87038722 | C | G |
| 16: 86898075 | T | C | 16: 86980174 | A | C | 16: 87038766 | A | G |
| 16: 86898196 | T | C | rs1000410797 | T | C | 16: 87038989 | A | T |
| rs557442272 | A | T | 16: 86980881 | T | C | rs550640679 | A | T |
| 16: 86898888 | C | G | rs144462375 | T | C | 16: 87039041 | A | G |
| 16: 86898988 | A | G | rs528795122 | A | G | 16: 87039042 | A | G |
| 16: 86899287 | T | C | 16: 86981136 | T | C | 16: 87039128 | T | C |
| rs1175347641 | A | G | 16: 86981236 | A | G | rs1486918147 | A | C |
| 16: 86899511 | C | G | rs1173801698 | T | C | 16: 87039215 | C | G |
| 16: 86899565 | T | C | 16: 86981532 | T | C | 16: 87039254 | T | C |
| 16: 86899694 | A | G | 16: 86981573 | A | C | 16: 87039315 | A | C |
| 16: 86899737 | A | G | rs192381862 | T | C | rs1182688713 | T | G |
| 16: 86899915 | A | G | rs184845079 | T | C | 16: 87039968 | C | G |
| 16: 86900000 | A | G | 16: 86981883 | A | G | 16: 87040011 | A | G |
| rs181695842 | A | C | rs1288852474 | C | G | 16: 87040315 | C | G |
| 16: 86900823 | T | C | rs1323290094 | A | C | 16: 87040384 | A | G |
| 16: 86901098 | T | C | rs1488531372 | C | G | 16: 87040385 | A | G |
| 16: 86901383 | T | G | 16: 86982309 | A | C | 16: 87040444 | A | G |
| 16: 86901516 | A | G | 16: 86982405 | A | G | 16: 87040507 | T | G |
| 16: 86902011 | T | C | rs1361523641 | A | C | 16: 87040617 | A | G |
| 16: 86902233 | A | C | 16: 86982623 | T | C | 16: 87040715 | T | C |
| rs1451119665 | A | G | rs920887761 | A | G | 16: 87040792 | C | G |
| rs570719230 | T | G | 16: 86982691 | C | G | rs1399008443 | A | G |
| 16: 86902650 | A | G | 16: 86982750 | A | G | 16: 87041491 | T | C |
| 16: 86902813 | A | G | 16: 86982872 | A | T | 16: 87041515 | T | C |
| 16: 86903194 | A | G | 16: 86982939 | T | C | 16: 87041589 | A | T |
| 16: 86903330 | T | G | rs1192600300 | A | C | 16: 87041644 | T | C |
| 16: 86904001 | T | C | rs1300179403 | A | G | 16: 87041776 | A | G |
| rs1053619652 | A | G | 16: 86983375 | C | G | 16: 87041870 | T | C |
| 16: 86904262 | A | C | 16: 86983425 | A | G | 16: 87042137 | A | G |
| rs992138533 | A | C | 16: 86983453 | A | C | 16: 87042173 | T | G |
| 16: 86905196 | T | C | rs984313947 | T | C | 16: 87042218 | A | C |
| rs1260323764 | A | C | 16: 86984273 | T | C | rs1420505731 | T | C |
| 16: 86905325 | A | G | 16: 86984442 | C | G | rs546560020 | A | G |
| 16: 86905411 | C | G | rs555618358 | A | T | 16: 87042708 | A | G |
| 16: 86905565 | A | G | rs756095161 | A | C | 16: 87043015 | C | G |
| 16: 86905649 | T | C | rs546816094 | A | T | 16: 87043029 | T | C |
| rs371022022 | T | C | 16: 86984595 | C | G | 16: 87043230 | A | G |
| 16: 86906109 | C | G | rs752186659 | T | C | rs1383913549 | T | C |
| rs529924069 | A | G | rs1428105561 | C | G | rs141173426 | T | C |
| rs759298228 | A | C | rs1169099072 | T | C | 16: 87043359 | A | G |
| 16: 86906401 | C | G | 16: 86985522 | A | G | 16: 87043822 | A | G |
| 16: 86906458 | A | G | rs937058916 | T | C | 16: 87043844 | T | G |
| 16: 86906662 | A | G | rs1015625822 | T | C | 16: 87043874 | T | G |
| 16: 86906829 | A | C | 16: 86986031 | A | G | 16: 87043916 | T | C |
| 16: 86906933 | T | C | 16: 86986035 | C | G | rs762590120 | A | G |
| rs1266864878 | A | G | 16: 86986257 | T | G | 16: 87044630 | T | C |
| 16: 86907297 | T | C | 16: 86986279 | A | T | 16: 87044755 | A | G |
| 16: 86907500 | T | C | 16: 86986792 | T | C | 16: 87044771 | A | G |
| rs117301054 | T | C | rs1310715726 | A | G | 16: 87045020 | T | C |
| 16: 86908200 | T | C | rs867245857 | A | C | 16: 87045177 | T | C |
| 16: 86908243 | A | G | 16: 86987193 | T | C | rs565945932 | C | G |
| 16: 86908470 | A | G | 16: 86987407 | T | C | 16: 87045284 | T | G |
| 16: 86908553 | A | G | rs1014265231 | C | G | rs1407469355 | T | G |
| 16: 86908652 | A | G | rs913827451 | T | C | rs1256811515 | A | G |
| rs190856976 | A | G | rs111625942 | A | G | rs1310066670 | A | G |
| 16: 86908807 | T | G | 16: 86987981 | A | G | rs1310536442 | A | C |
| rs776043853 | A | G | 16: 86987991 | A | G | 16: 87045712 | C | G |
| 16: 86909239 | T | G | 16: 86988098 | T | G | 16: 87045728 | A | C |
| 16: 86909301 | T | C | 16: 86988186 | A | G | rs1447324903 | A | G |
| rs1347596089 | T | C | 16: 86988256 | T | C | 16: 87046012 | A | C |
| 16: 86909604 | T | C | rs906188144 | C | G | rs1228256534 | A | G |
| 16: 86909900 | A | G | 16: 86988417 | T | C | rs879817638 | A | T |
| rs1007884032 | T | C | rs1332035826 | T | G | 16: 87046188 | T | C |
| 16: 86910194 | T | C | rs1397046427 | A | G | 16: 87046246 | T | C |
| 16: 86910387 | A | G | 16: 86989268 | A | G | 16: 87046322 | T | G |
| 16: 86910392 | T | C | rs1228841851 | A | G | 16: 87046327 | A | G |
| rs1027657048 | A | C | rs1051605126 | A | T | 16: 87046363 | A | C |
| 16: 86911311 | A | T | 16: 86989603 | A | G | rs1358699949 | C | G |
| rs542058207 | T | C | 16: 86989610 | A | G | 16: 87046632 | A | C |
| 16: 86912043 | T | C | 16: 86989746 | A | C | rs935935786 | T | C |
| 16: 86912155 | A | G | 16: 86989868 | A | G | 16: 87047185 | T | C |
| rs1387223120 | A | C | 16: 86990084 | C | G | 16: 87047368 | A | G |
| 16: 86912279 | A | G | 16: 86990133 | T | G | 16: 87047405 | T | C |
| 16: 86912585 | T | C | 16: 86990173 | T | C | rs1297747786 | A | G |
| rs915865783 | T | C | 16: 86990194 | A | G | 16: 87047495 | T | C |
| 16: 86912887 | A | G | 16: 86990322 | T | C | 16: 87047545 | T | G |
| 16: 86912940 | T | G | 16: 86990402 | A | G | 16: 87047968 | A | C |
| 16: 86912941 | C | G | 16: 86990619 | T | C | 16: 87047978 | T | G |
| 16: 86913229 | T | G | rs781306937 | T | C | 16: 87048080 | A | T |
| 16: 86914430 | T | C | 16: 86991422 | T | C | 16: 87048419 | A | C |
| 16: 86914474 | C | G | 16: 86991714 | A | G | 16: 87048558 | A | C |
| 16: 86914500 | A | G | rs1408473441 | T | C | 16: 87048684 | T | G |
| 16: 86914668 | T | C | 16: 86991938 | A | G | 16: 87048842 | A | G |
| 16: 86914671 | T | C | 16: 86992246 | A | G | rs1426895289 | A | G |
| 16: 86914742 | A | G | 16: 86992536 | A | G | 16: 87049055 | T | C |
| 16: 86915098 | T | C | 16: 86992636 | T | C | rs1045282486 | C | G |
| 16: 86915615 | T | C | 16: 86993202 | A | G | 16: 87049362 | A | C |
| 16: 86915973 | T | C | 16: 86993229 | T | G | 16: 87049396 | T | C |
| 16: 86916029 | A | G | 16: 86993310 | T | C | 16: 87049427 | A | G |
| rs990623883 | T | C | 16: 86993623 | T | C | 16: 87049438 | A | C |
| 16: 86916287 | A | C | 16: 86994022 | A | T | 16: 87049473 | A | G |
| 16: 86916377 | A | C | 16: 86994356 | C | G | 16: 87050050 | A | G |
| 16: 86916495 | A | G | 16: 86994457 | A | G | rs1290878083 | T | C |
| rs1011740994 | C | G | 16: 86994682 | A | C | 16: 87050317 | A | G |
| 16: 86916739 | A | G | 16: 86994969 | A | C | 16: 87050368 | A | C |
| rs997340060 | T | C | 16: 86995183 | A | C | 16: 87050379 | C | G |
| rs1439542611 | A | G | 16: 86995419 | T | C | rs535913355 | A | G |
| 16: 86917117 | T | C | 16: 86995513 | C | G | 16: 87050635 | A | C |
| rs771273406 | A | T | 16: 86995654 | C | G | 16: 87050765 | T | G |
| 16: 86917171 | C | G | 16: 86995690 | T | G | 16: 87050910 | T | C |
| 16: 86917201 | T | C | 16: 86995883 | T | C | rs950629915 | T | C |
| 16: 86917296 | C | G | 16: 86995964 | T | C | 16: 87051325 | C | G |
| rs75258357 | T | C | 16: 86996284 | A | G | 16: 87051698 | A | G |
| rs1360557128 | T | C | 16: 86996296 | T | C | rs1471577348 | T | G |
| 16: 86918113 | A | G | 16: 86996308 | A | G | 16: 87052013 | C | G |
| 16: 86918194 | T | C | 16: 86996352 | A | G | rs1012707193 | A | C |
| rs9925927 | A | G | 16: 86996369 | T | G | 16: 87052687 | A | G |
| rs112002378 | T | C | 16: 86996472 | A | G | rs561188614 | T | G |
| rs1391594255 | T | C | 16: 86996569 | A | G | 16: 87052937 | A | C |
| rs113705222 | T | C | 16: 86996662 | A | G | 16: 87052977 | T | C |
| 16: 86918734 | C | G | rs1314290340 | A | G | 16: 87053094 | C | G |
| 16: 86918823 | C | G | 16: 86997124 | T | C | 16: 87053159 | C | G |
| rs1034356397 | C | G | 16: 86997217 | A | G | 16: 87053205 | A | T |
| 16: 86918906 | C | G | 16: 86997247 | A | G | 16: 87053257 | T | C |
| 16: 86918918 | A | G | 16: 86997248 | T | G | 16: 87053261 | A | G |
| 16: 86918926 | T | C | 16: 86997497 | T | C | 16: 87053359 | C | G |
| 16: 86918942 | C | G | rs1227586934 | C | G | 16: 87053416 | A | G |
| 16: 86919099 | T | C | rs8054864 | A | G | rs927153103 | T | C |
| rs765111364 | T | C | 16: 86998046 | A | G | 16: 87053713 | A | G |
| 16: 86919486 | A | G | rs186678346 | A | G | rs1220157345 | T | G |
| 16: 86919509 | A | C | rs954252263 | A | G | rs925336206 | T | C |
| rs117362436 | C | G | 16: 86998611 | T | C | 16: 87053869 | T | G |
| 16: 86919609 | A | G | 16: 86998879 | C | G | 16: 87053923 | A | C |
| 16: 86919612 | T | C | 16: 86998944 | A | C | 16: 87053953 | A | G |
| 16: 86919668 | C | G | 16: 86998982 | C | G | 16: 87054119 | C | G |
| 16: 86919765 | A | C | 16: 86999588 | T | C | 16: 87054437 | A | C |
| rs1012738151 | T | G | rs572284262 | A | G | 16: 87054556 | C | G |
| 16: 86919774 | T | C | 16: 87000330 | T | C | 16: 87054579 | A | C |
| 16: 86920089 | T | C | 16: 87000406 | T | C | 16: 87054705 | A | T |
| 16: 86920181 | T | C | rs951563268 | T | C | rs1324392405 | T | C |
| 16: 86920349 | A | T | rs557886259 | T | C | 16: 87055006 | A | G |
| 16: 86920398 | T | C | 16: 87000753 | A | G | 16: 87055052 | A | C |
| 16: 86920511 | T | C | 16: 87000813 | T | C | 16: 87055076 | A | T |
| 16: 86920523 | A | G | 16: 87000951 | T | C | rs961296653 | T | C |
| 16: 86920642 | T | C | 16: 87000963 | A | G | 16: 87055387 | A | G |
| 16: 86920724 | T | C | 16: 87001044 | T | C | rs956089610 | A | G |
| 16: 86921048 | A | G | 16: 87001063 | C | G | 16: 87055542 | T | G |
| 16: 86921192 | T | C | rs917215932 | A | C | 16: 87056084 | A | G |
| 16: 86921278 | A | G | 16: 87001102 | T | C | 16: 87056584 | A | G |
| 16: 86921307 | A | G | 16: 87001281 | C | G | 16: 87056588 | T | G |
| rs906007722 | T | G | 16: 87001432 | C | G | 16: 87056710 | T | C |
| 16: 86921487 | C | G | rs952531358 | A | T | rs189530621 | T | C |
| 16: 86921530 | T | C | rs542055861 | A | G | 16: 87057046 | T | C |
| rs1176880761 | A | G | rs1287859797 | A | G | 16: 87057358 | C | G |
| 16: 86921847 | C | G | 16: 87002526 | A | G | 16: 87057703 | T | C |
| 16: 86921916 | T | C | rs1227105839 | A | G | rs918216461 | A | G |
| 16: 86921969 | T | G | rs1010604706 | A | G | 16: 87057998 | T | G |
| 16: 86921991 | T | C | rs569005590 | T | G | 16: 87058814 | T | C |
| 16: 86922010 | A | C | rs991851629 | A | T | rs1467203081 | T | C |
| 16: 86922292 | A | G | 16: 87003023 | A | T | 16: 87059060 | T | G |
| 16: 86922387 | A | C | 16: 87003593 | T | C | 16: 87059323 | A | T |
| 16: 86922534 | A | G | rs1373022079 | C | G | 16: 87059336 | T | G |
| 16: 86922661 | A | G | rs1471846187 | A | G | 16: 87059395 | A | G |
| rs8045541 | T | C | 16: 87004342 | C | G | 16: 87059497 | A | G |
| 16: 86923608 | A | T | rs1488032145 | C | G | 16: 87059527 | C | G |
| 16: 86923785 | T | C | 16: 87004792 | A | G | 16: 87059603 | A | T |
| 16: 86923791 | C | G | 16: 87005086 | A | G | 16: 87059642 | T | C |
| 16: 86923820 | A | G | 16: 87005231 | A | G | 16: 87059734 | T | C |
| rs939792660 | T | C | 16: 87005351 | T | C | 16: 87059764 | A | T |
| 16: 86924766 | A | G | 16: 87005397 | C | G | 16: 87059950 | A | C |
| 16: 86925142 | A | G | 16: 87005441 | C | G | 16: 87059995 | A | G |
| 16: 86925168 | A | G | rs764701453 | T | C | 16: 87060126 | T | C |
| 16: 86925454 | T | C | rs1398758500 | T | C | rs990076676 | A | G |
| rs1301180485 | T | C | rs1384807000 | T | C | rs1320769447 | A | G |
| rs910591303 | T | C | rs1233061487 | A | G | 16: 87060311 | T | C |
| 16: 86925689 | T | C | rs1224401385 | T | G | rs1406387983 | C | G |
| 16: 86925917 | T | C | 16: 87005894 | A | G | 16: 87060629 | A | G |
| rs964166784 | T | C | 16: 87006036 | C | G | 16: 87060646 | T | C |
| 16: 86926098 | C | G | 16: 87006226 | A | C | 16: 87060905 | T | G |
| rs533148900 | T | C | 16: 87006267 | A | G | rs1440993666 | A | G |
| 16: 86926192 | T | G | 16: 87006458 | T | C | 16: 87061023 | C | G |
| 16: 86926315 | T | C | 16: 87006475 | A | G | 16: 87061782 | T | C |
| 16: 86926389 | T | C | 16: 87006557 | T | C | rs1186215838 | T | C |
| rs1192721354 | T | C | 16: 87006665 | T | C | 16: 87062046 | T | C |
| 16: 86926445 | T | C | rs1267033635 | A | G | 16: 87062205 | A | G |
| 16: 86926467 | A | G | rs954851140 | T | C | rs1381581499 | A | G |
| 16: 86926569 | C | G | 16: 87006881 | T | C | rs77107665 | A | G |
| rs1401483330 | A | G | 16: 87006882 | C | G | 16: 87062530 | A | C |
| rs912842522 | A | G | 16: 87006982 | A | G | 16: 87062542 | C | G |
| rs555477874 | A | C | rs1341007540 | C | G | rs1013798422 | A | G |
| 16: 86926872 | T | C | 16: 87007157 | A | C | rs1480591049 | T | C |
| 16: 86926894 | A | G | rs1290143275 | T | C | 16: 87062832 | C | G |
| rs537026588 | T | C | rs886151857 | A | T | 16: 87062944 | C | G |
| 16: 86927370 | A | T | 16: 87007198 | T | C | 16: 87063060 | T | C |
| 16: 86927603 | T | C | 16: 87007433 | A | C | 16: 87063190 | A | G |
| 16: 86927608 | T | G | 16: 87007492 | T | C | rs1382908798 | T | C |
| 16: 86927672 | T | C | rs774478886 | C | G | rs1156904470 | A | G |
| 16: 86927674 | C | G | rs958148065 | C | G | 16: 87063327 | A | G |
| 16: 86927677 | T | C | 16: 87007600 | A | C | rs1345887245 | A | G |
| 16: 86927732 | A | T | 16: 87007631 | C | G | 16: 87063437 | A | G |
| 16: 86927744 | T | C | rs1236880345 | A | T | 16: 87063751 | A | G |
| 16: 86927850 | A | C | 16: 87008067 | T | C | rs1468362487 | A | C |
| 16: 86927904 | A | C | 16: 87008077 | T | G | 16: 87063876 | T | C |
| 16: 86928135 | T | C | 16: 87008174 | T | C | 16: 87064225 | T | C |
| 16: 86928141 | T | C | 16: 87008319 | A | G | 16: 87064376 | A | G |
| 16: 86928408 | A | G | 16: 87008549 | A | G | 16: 87064385 | A | T |
| 16: 86928435 | T | G | 16: 87008561 | T | C | 16: 87064724 | T | C |
| 16: 86928543 | A | C | rs1037257298 | A | G | 16: 87065033 | A | G |
| 16: 86928681 | C | G | 16: 87008781 | T | C | 16: 87065318 | T | C |
| 16: 86929002 | A | G | 16: 87008956 | A | G | 16: 87065348 | A | C |
| 16: 86929044 | T | C | rs1402520680 | T | C | rs1282775300 | T | C |
| 16: 86929062 | A | C | 16: 87009288 | C | G | 16: 87065455 | A | G |
| 16: 86929282 | T | C | rs946510554 | T | C | 16: 87065603 | A | G |
| 16: 86929302 | A | G | 16: 87009486 | A | G | 16: 87065675 | C | G |
| 16: 86929329 | A | G | 16: 87009542 | A | G | 16: 87065850 | A | G |
| 16: 86929340 | A | G | 16: 87009632 | T | G | 16: 87066201 | T | C |
| rs181809264 | T | G | 16: 87009674 | C | G | rs937947854 | A | G |
| 16: 86929731 | A | C | rs970399090 | A | G | 16: 87066499 | A | G |
| rs1246349427 | C | G | 16: 87010387 | T | C | rs909916315 | T | C |
| 16: 86930152 | C | G | 16: 87011086 | C | G | 16: 87066659 | T | C |
| 16: 86930189 | A | G | 16: 87011159 | A | G | 16: 87067192 | C | G |
| rs1238349257 | T | C | 16: 87011162 | T | G | rs1349945892 | C | G |
| 16: 86930650 | C | G | rs1166644606 | T | C | 16: 87068409 | C | G |
| 16: 86930695 | T | C | rs182434511 | A | C | 16: 87068463 | T | C |
| 16: 86931350 | A | G | rs539204047 | T | G | 16: 87068506 | A | G |
| rs1049797119 | A | G | 16: 87011751 | C | G | 16: 87068626 | A | G |
| rs1254257971 | T | G | 16: 87011802 | T | C | 16: 87068634 | A | G |
| 16: 86932687 | T | C | 16: 87011918 | T | C | 16: 87068876 | A | G |
| 16: 86932754 | T | C | 16: 87012097 | T | C | 16: 87068932 | C | G |
| 16: 86932895 | A | G | rs1338389978 | A | G | 16: 87068975 | A | G |
| 16: 86932973 | T | C | 16: 87012164 | A | G | 16: 87069194 | T | G |
| rs891084812 | A | G | 16: 87012264 | A | G | 16: 87069216 | A | G |
| 16: 86933771 | A | C | rs985907864 | A | C | 16: 87069386 | T | G |
| 16: 86933779 | C | G | 16: 87012498 | T | C | 16: 87069462 | T | C |
| 16: 86933894 | C | G | 16: 87012648 | T | G | rs1182679150 | A | C |
| 16: 86934448 | T | C | 16: 87012702 | A | G | rs1480870914 | T | C |
| 16: 86934569 | T | C | 16: 87012745 | T | C | rs1158231012 | T | G |
| 16: 86934612 | T | C | rs960564209 | C | G | 16: 87070155 | C | G |
| 16: 86934677 | T | C | 16: 87012911 | A | T | 16: 87070172 | T | C |
| 16: 86935809 | A | G | 16: 87012944 | A | G | 16: 87070208 | A | C |
| 16: 86935914 | C | G | rs556669351 | C | G | 16: 87070496 | T | C |
| rs1030523191 | T | C | 16: 87013445 | T | G | 16: 87070781 | T | C |
| rs1393236689 | T | C | 16: 87013586 | T | C | 16: 87071161 | A | G |
| 16: 86936060 | A | G | 16: 87013641 | A | C | 16: 87071346 | A | G |
| 16: 86936266 | A | G | 16: 87013657 | T | C | 16: 87071479 | A | C |
| 16: 86936302 | A | G | 16: 87013678 | C | G | 16: 87071662 | A | G |
| 16: 86936502 | T | C | 16: 87013709 | T | G | 16: 87071857 | A | G |
| 16: 86936665 | A | T | rs1189071227 | T | C | 16: 87072587 | T | C |
| 16: 86936666 | T | C | 16: 87013890 | A | G | 16: 87072676 | C | G |
| 16: 86937260 | A | T | 16: 87014057 | A | G | rs975408631 | A | G |
| rs1160207411 | T | C | rs1243626937 | T | G | 16: 87073000 | C | G |
| 16: 86937543 | A | G | rs1417191101 | A | G | 16: 87073152 | T | G |
| 16: 86937691 | A | G | 16: 87014655 | C | G | rs1397858680 | T | G |
| rs538936639 | C | G | rs978041536 | A | T | 16: 87073550 | A | C |
| 16: 86937892 | A | G | rs1026007052 | T | C | 16: 87073635 | A | G |
| 16: 86937998 | A | G | 16: 87015491 | T | C | rs1015407008 | A | G |
| rs1421159539 | T | G | 16: 87015520 | A | G | 16: 87074902 | T | C |
| 16: 86938234 | A | G | 16: 87015524 | A | C | 16: 87074906 | T | C |
| 16: 86938416 | A | G | rs987325876 | A | G | 16: 87075000 | A | G |
| 16: 86938792 | A | T | rs1228312345 | A | G | rs1358806005 | A | G |
| 16: 86938895 | T | C | 16: 87015617 | A | G | rs1211264261 | A | C |
| 16: 86938917 | C | G | rs528444284 | A | T | 16: 87075398 | T | C |
| 16: 86939550 | C | G | 16: 87016016 | A | G | 16: 87075494 | T | G |
| 16: 86939691 | C | G | 16: 87016532 | T | C | rs1030793604 | T | C |
| 16: 86940018 | A | G | rs1461825732 | C | G | 16: 87075646 | A | G |
| rs1378853459 | C | G | 16: 87017075 | A | C | 16: 87075851 | C | G |
| rs1219295665 | A | G | rs1162881634 | T | C | 16: 87076052 | C | G |
| 16: 86940385 | C | G | 16: 87018465 | T | C | rs1365970841 | A | C |
| 16: 86940478 | A | G | 16: 87018807 | T | C | 16: 87076134 | A | G |
| 16: 86940721 | T | C | 16: 87019166 | T | C | 16: 87076572 | T | G |
| 16: 86940722 | A | G | 16: 87019415 | T | C | rs1323866248 | T | C |
| 16: 86940919 | C | G | 16: 87019735 | A | C | 16: 87077189 | C | G |
| 16: 86940957 | A | T | rs1468931604 | T | G | rs1297297764 | A | G |
| 16: 86941095 | T | G | 16: 87019981 | C | G | 16: 87077363 | A | G |
| 16: 86941380 | T | C | 16: 87020014 | A | G | 16: 87077399 | T | C |
| 16: 86941550 | C | G | rs1355867363 | A | G | 16: 87077722 | T | C |
| 16: 86941556 | A | G | rs1441248011 | C | G | 16: 87077823 | T | C |
| 16: 86941833 | T | G | rs1390281104 | T | C | rs146495273 | C | G |
| 16: 86942017 | C | G | 16: 87020278 | A | C | rs1278387504 | A | G |
| rs567149609 | T | C | 16: 87020352 | C | G | rs143486902 | A | G |
| 16: 86943361 | A | G | 16: 87020627 | C | G | 16: 87079002 | T | C |
| rs569374812 | T | C | rs1401774423 | A | G | 16: 87079255 | A | G |
| 16: 86944627 | T | C | rs183303920 | T | C | 16: 87079491 | T | C |
| rs1242826297 | T | G | 16: 87020938 | T | C | rs74039608 | A | T |
| rs1230046408 | A | G | 16: 87021299 | T | C | rs1326242591 | A | C |
| 16: 86945763 | A | C | 16: 87021437 | A | C | 16: 87080429 | T | G |
| rs1441061528 | T | C | 16: 87021527 | A | G | 16: 87080483 | C | G |
| rs375952251 | T | C | rs926989364 | T | C | 16: 87080607 | C | G |
| 16: 86947471 | A | G | rs1034445385 | C | G | 16: 87080636 | A | G |
| rs549374405 | T | C | rs1338994035 | A | G | 16: 87080714 | A | G |
| 16: 86947685 | A | G | 16: 87022127 | A | G | rs117641916 | A | T |
| 16: 86947848 | T | C | rs971876648 | A | G | 16: 87080971 | A | C |
| rs1295818635 | T | C | 16: 87022342 | C | G | 16: 87081125 | T | G |
| rs772775468 | T | G | 16: 87022476 | A | G | rs80235720 | A | G |
| 16: 86949318 | A | C | 16: 87022764 | T | C | 16: 87081582 | T | C |
| rs1241358472 | T | G | rs553336507 | T | G | 16: 87081829 | A | G |
| 16: 86949463 | C | G | rs1255531264 | C | G | rs61631820 | T | C |
| 16: 86950418 | T | C | 16: 87023444 | T | C | rs1194626789 | A | T |
| 16: 86950613 | A | G | rs1252880403 | A | G | 16: 87083202 | A | C |
| 16: 86950759 | C | G | rs373830778 | A | G | 16: 87084185 | A | G |
| 16: 86951109 | T | C | 16: 87023862 | A | C | 16: 87084423 | T | C |
| 16: 86951220 | T | C | 16: 87024168 | T | C | 16: 87084701 | T | C |
| 16: 86951267 | T | C | 16: 87024412 | C | G | rs1375643975 | T | G |
| 16: 86951280 | T | C | rs1299246266 | C | G | 16: 87085067 | T | C |
| 16: 86951284 | T | C | 16: 87024663 | A | G | 16: 87085158 | C | G |
| 16: 86951339 | A | G | 16: 87024861 | T | C | 16: 87085165 | A | T |
| rs924636964 | T | C | 16: 87024993 | C | G | 16: 87085237 | A | C |
| rs1362408747 | T | C | 16: 87025112 | A | G | rs1263194384 | A | G |
| 16: 86951807 | C | G | 16: 87025127 | A | T | 16: 87085348 | A | G |
| 16: 86952035 | T | C | 16: 87025636 | T | C | 16: 87085366 | A | G |
| 16: 86952151 | T | C | rs1013946700 | T | C | 16: 87085394 | A | G |
| 16: 86952227 | A | G | 16: 87025993 | A | G | 16: 87085778 | T | C |
| rs531975811 | T | G | 16: 87026073 | T | C | 16: 87085927 | C | G |
| rs769646666 | T | C | 16: 87026097 | T | C | rs1423759 | C | G |
| rs568154925 | A | T | rs981897813 | A | G | 16: 87086453 | T | C |
| 16: 86953077 | T | C | 16: 87026463 | T | C | rs1048846531 | A | G |
| rs1379135826 | T | C | rs1323599762 | A | T | 16: 87086492 | T | C |
| rs1385031136 | A | G | 16: 87026497 | C | G | 16: 87086555 | T | C |
| 16: 86954485 | T | G | rs1297495674 | C | G | rs1046411656 | C | G |
| rs868821313 | T | C | 16: 87026720 | T | C | rs1011236574 | T | C |
| rs1407245453 | T | C | 16: 87026786 | A | C | 16: 87087379 | C | G |
| 16: 86955023 | T | C | 16: 87026838 | T | G | 16: 87087512 | A | G |
| 16: 86955287 | C | G | 16: 87027059 | A | G | rs1303871767 | C | G |
| 16: 86956304 | T | C | 16: 87027124 | T | G | 16: 87088176 | A | G |
| rs1449406385 | C | G | 16: 87027355 | A | G | 16: 87088293 | A | G |
| 16: 86956369 | T | C | 16: 87027671 | T | C | rs1248471838 | T | C |
| rs370277799 | T | C | 16: 87027680 | C | G | 16: 87088381 | C | G |
| 16: 86956445 | C | G | 16: 87027693 | T | C | 16: 87088531 | A | T |
| 16: 86956576 | A | T | 16: 87027702 | A | G | 16: 87088709 | T | C |
| rs957279524 | A | C | 16: 87027717 | A | G | rs1422575614 | T | C |
| 16: 86958876 | T | C | rs924511841 | T | C | rs1191055809 | T | G |
| 16: 86958976 | T | C | rs1048580939 | T | C | rs1468670401 | A | T |
| 16: 86959103 | T | C | 16: 87027848 | A | T | 16: 87089825 | A | G |
| 16: 86959106 | A | G | 16: 87027860 | T | C | 16: 87089842 | T | G |
| 16: 86959497 | A | G | rs118154610 | T | C | 16: 87090206 | T | G |
| 16: 86959560 | A | T | rs1395611933 | C | G | 16: 87090641 | A | G |
| rs748235051 | T | C | 16: 87027974 | C | G | rs935957589 | T | C |
| 16: 86959650 | A | G | 16: 87027987 | A | G | 16: 87091139 | A | G |
| rs946624660 | A | G | rs998360174 | A | G | 16: 87091199 | A | G |
| 16: 86960492 | T | C | rs933752879 | A | C | 16: 87091538 | A | G |
| 16: 86961436 | T | C | 16: 87028625 | A | G | 16: 87091739 | A | G |
| rs1007103093 | A | G | 16: 87028633 | T | C | 16: 87091785 | C | G |
| rs1267095348 | A | G | 16: 87028808 | A | C | 16: 87091867 | T | G |
| 16: 86962607 | A | T | rs11648124 | A | G | | | |
| rs1220961528 | A | G | 16: 87029010 | T | C | | | |

| SEQ | | | | SEQ | | | |
|---|---|---|---|---|---|---|---|
| ID | | chr_ | chr_ | ID | | chr_ | chr_ |
| NO: | Sequence | start | end | NO: | Sequence | start | end |
| 2603 | CATCTGTGAGTTCCCG | 42044105 | 42044120 | 2725 | GTGGCTCTGATGTGACTCTT | 42041461 | 42041480 |
| 2604 | ATCTGTGAGTTCCCGT | 42044106 | 42044121 | 2726 | TGGCTCTGATGTGACTCTTC | 42041462 | 42041481 |
| 2605 | TGTGAGTTCCCGTCCA | 42044109 | 42044124 | 2727 | GGCTCTGATGTGACTCTTCT | 42041463 | 42041482 |
| 2606 | CATGGTTGGGCGTCTG | 42046518 | 42046533 | 2728 | GCTCTGATGTGACTCTTCTG | 42041464 | 42041483 |
| 2607 | ATGGTTGGGCGTCTGC | 42046519 | 42046534 | 2729 | TCTGATGTGACTCTTCTGTG | 42041466 | 42041485 |
| 2608 | TGGTTGGGCGTCTGCA | 42046520 | 42046535 | 2730 | CTGATGTGACTCTTCTGTGG | 42041467 | 42041486 |
| 2609 | CAACACGGAGACCACA | 42046776 | 42046791 | 2731 | TGATGTGACTCTTCTGTGGC | 42041468 | 42041487 |
| 2610 | ACACGGAGACCACATT | 42046778 | 42046793 | 2732 | GATGTGACTCTTCTGTGGCT | 42041469 | 42041488 |
| 2611 | CACGGAGACCACATTT | 42046779 | 42046794 | 2733 | GACGTGAAGCAAAGACACTC | 42042245 | 42042264 |
| 2612 | TTAGGTCACTATGGGA | 42047148 | 42047163 | 2734 | TGTGTCTCAATTAATTTGCC | 42042751 | 42042770 |
| 2613 | TAGGTCACTATGGGAG | 42047149 | 42047164 | 2735 | GTGTCTCAATTAATTTGCCG | 42042752 | 42042771 |
| 2614 | GTCTGTCGGGTAATTG | 42048039 | 42048054 | 2736 | TGTCTCAATTAATTTGCCGT | 42042753 | 42042772 |
| 2615 | TCTGTCGGGTAATTGA | 42048040 | 42048055 | 2737 | GTCTCAATTAATTTGCCGTG | 42042754 | 42042773 |
| 2616 | CTGTCGGGTAATTGAA | 42048041 | 42048056 | 2738 | TCTCAATTAATTTGCCGTGT | 42042755 | 42042774 |
| 2617 | TGTCGGGTAATTGAAT | 42048042 | 42048057 | 2739 | CTCAATTAATTTGCCGTGTA | 42042756 | 42042775 |
| 2618 | GTCGGGTAATTGAATG | 42048043 | 42048058 | 2740 | GCCTGCTCTGGTGGACGGTA | 42043323 | 42043342 |
| 2619 | TCGGGTAATTGAATGA | 42048044 | 42048059 | 2741 | GGCAGCAGGACTCTGAGGAC | 42043440 | 42043459 |
| 2620 | CGGGTAATTGAATGAC | 42048045 | 42048060 | 2742 | GGCACAAGGATTGGTGGTGT | 42043631 | 42043650 |
| 2621 | GCTTGTCACGGGTCAG | 42048711 | 42048726 | 2743 | GCACAAGGATTGGTGGTGTC | 42043632 | 42043651 |
| 2622 | CTTGTCACGGGTCAGT | 42048712 | 42048727 | 2744 | CACAAGGATTGGTGGTGTCT | 42043633 | 42043652 |
| 2623 | TAAGTCCATGTTTGCG | 42049526 | 42049541 | 2745 | ACAAGGATTGGTGGTGTCTG | 42043634 | 42043653 |
| 2624 | AAGTCCATGTTTGCGG | 42049527 | 42049542 | 2746 | CAAGGATTGGTGGTGTCTGC | 42043635 | 42043654 |
| 2625 | AGTCCATGTTTGCGGG | 42049528 | 42049543 | 2747 | AAGGATTGGTGGTGTCTGCC | 42043636 | 42043655 |
| 2626 | GTCCATGTTTGCGGGA | 42049529 | 42049544 | 2748 | AGGATTGGTGGTGTCTGCCT | 42043637 | 42043656 |
| 2627 | TCCATGTTTGCGGGAA | 42049530 | 42049545 | 2749 | GGATTGGTGGTGTCTGCCTG | 42043638 | 42043657 |
| 2628 | CCATGTTTGCGGGAAC | 42049531 | 42049546 | 2750 | GATTGGTGGTGTCTGCCTGA | 42043639 | 42043658 |
| 2629 | CATGTTTGCGGGAACC | 42049532 | 42049547 | 2751 | ATTGGTGGTGTCTGCCTGAA | 42043640 | 42043659 |
| 2630 | ATGTTTGCGGGAACCT | 42049533 | 42049548 | 2752 | TTGGTGGTGTCTGCCTGAAG | 42043641 | 42043660 |
| 2631 | TGTTTGCGGGAACCTA | 42049534 | 42049549 | 2753 | CATCTGTGAGTTCCCGTCCA | 42044105 | 42044124 |
| 2632 | GTTTGCGGGAACCTAA | 42049535 | 42049550 | 2754 | CCCACCTTCAGACTTTCAAG | 42045415 | 42045434 |
| 2633 | TTTGCGGGAACCTAAG | 42049536 | 42049551 | 2755 | CCACCTTCAGACTTTCAAGA | 42045416 | 42045435 |
| 2634 | GGCCACGCTCTTTGAT | 42051783 | 42051798 | 2756 | TTGGCTGCGTGTCCTGGTGT | 42045801 | 42045820 |
| 2635 | GATGCGATGTTGGTCA | 42051931 | 42051946 | 2757 | TGGCTGCGTGTCCTGGTGTC | 42045802 | 42045821 |
| 2636 | GAGATGATGTGATCGC | 42052025 | 42052040 | 2758 | GGCTGCGTGTCCTGGTGTCT | 42045803 | 42045822 |
| 2637 | AGATGATGTGATCGCC | 42052026 | 42052041 | 2759 | GCTGCGTGTCCTGGTGTCTG | 42045804 | 42045823 |
| 2638 | GATGATGTGATCGCCC | 42052027 | 42052042 | 2760 | CTGCGTGTCCTGGTGTCTGT | 42045805 | 42045824 |
| 2639 | ATGATGTGATCGCCCT | 42052028 | 42052043 | 2761 | TGCGTGTCCTGGTGTCTGTC | 42045806 | 42045825 |
| 2640 | TGATGTGATCGCCCTT | 42052029 | 42052044 | 2762 | GCGTGTCCTGGTGTCTGTCA | 42045807 | 42045826 |
| 2641 | GATGTGATCGCCCTTG | 42052030 | 42052045 | 2763 | TACAGTCAGCTCTTCTTGAG | 42045918 | 42045937 |
| 2642 | ATGTGATCGCCCTTGG | 42052031 | 42052046 | 2764 | CTCTTCTTGAGTATTGGAAT | 42045927 | 42045946 |
| 2643 | GCTGTTCGCAACTTGG | 42054047 | 42054062 | 2765 | CAGTGGTTCTGTATGTCTAA | 42046015 | 42046034 |
| 2644 | CTGTTCGCAACTTGGT | 42054048 | 42054063 | 2766 | AGTGGTTCTGTATGTCTAAC | 42046016 | 42046035 |
| 2645 | TGTTCGCAACTTGGTA | 42054049 | 42054064 | 2767 | GTGGTTCTGTATGTCTAACT | 42046017 | 42046036 |
| 2646 | GTTCGCAACTTGGTAC | 42054050 | 42054065 | 2768 | TGGTTCTGTATGTCTAACTC | 42046018 | 42046037 |
| 2647 | GCAACTTGGTACGAAT | 42054054 | 42054069 | 2769 | GGTTCTGTATGTCTAACTCT | 42046019 | 42046038 |
| 2648 | CAACTTGGTACGAATG | 42054055 | 42054070 | 2770 | GTTCTGTATGTCTAACTCTA | 42046020 | 42046039 |
| 2649 | ATGGCTCAATTTACATGACA | 42037644 | 42037663 | 2771 | TGTATGTCTAACTCTATCTG | 42046024 | 42046043 |
| 2650 | CACGTCCAGAATATGCAAAT | 42037662 | 42037681 | 2772 | GTATGTCTAACTCTATCTGA | 42046025 | 42046044 |
| 2651 | ACGTCCAGAATATGCAAATT | 42037663 | 42037682 | 2773 | CTTGTTTCTTAAAGGATCTG | 42047100 | 42047119 |
| 2652 | GGTGAATTGAATAGCATGTG | 42037862 | 42037881 | 2774 | TTGTTTCTTAAAGGATCTGG | 42047101 | 42047120 |
| 2653 | CAACTGAACAAATGCGTGGC | 42038186 | 42038205 | 2775 | TGTTTCTTAAAGGATCTGGA | 42047102 | 42047121 |
| 2654 | AACTGAACAAATGCGTGGCA | 42038187 | 42038206 | 2776 | GTTTCTTAAAGGATCTGGAC | 42047103 | 42047122 |
| 2655 | ACTGAACAAATGCGTGGCAG | 42038188 | 42038207 | 2777 | GACTTTAGGTCACTATGGGA | 42047144 | 42047163 |
| 2656 | CTGAACAAATGCGTGGCAGG | 42038189 | 42038208 | 2778 | ACTTTAGGTCACTATGGGAG | 42047145 | 42047164 |
| 2657 | CAAATGCGTGGCAGGAATAT | 42038194 | 42038213 | 2779 | CTTTAGGTCACTATGGGAGT | 42047146 | 42047165 |
| 2658 | TGCGTGGCAGGAATATAAAC | 42038198 | 42038217 | 2780 | TTTAGGTCACTATGGGAGTC | 42047147 | 42047166 |
| 2659 | ACAACAGATGTTCATTTCTC | 42039204 | 42039223 | 2781 | CTTAAGCATCTCTAGTCCAA | 42047993 | 42048012 |
| 2660 | CAACAGATGTTCATTTCTCA | 42039205 | 42039224 | 2782 | TTAAGCATCTCTAGTCCAAA | 42047994 | 42048013 |
| 2661 | AACAGATGTTCATTTCTCAC | 42039206 | 42039225 | 2783 | TAAGCATCTCTAGTCCAAAC | 42047995 | 42048014 |
| 2662 | ACAGATGTTCATTTCTCACA | 42039207 | 42039226 | 2784 | ATGTCTGTCGGGTAATTGAA | 42048037 | 42048056 |
| 2663 | CAGATGTTCATTTCTCACAG | 42039208 | 42039227 | 2785 | TGTCTGTCGGGTAATTGAAT | 42048038 | 42048057 |
| 2664 | AGATGTTCATTTCTCACAGT | 42039209 | 42039228 | 2786 | GTCTGTCGGGTAATTGAATG | 42048039 | 42048058 |
| 2665 | GATGTTCATTTCTCACAGTT | 42039210 | 42039229 | 2787 | TCTGTCGGGTAATTGAATGA | 42048040 | 42048059 |
| 2666 | TTACAGCAGTGACGGGAAAC | 42039556 | 42039575 | 2788 | GTCGGGTAATTGAATGACAT | 42048043 | 42048062 |
| 2667 | TACAGCAGTGACGGGAAACT | 42039557 | 42039576 | 2789 | GATTCTGAAGTCATTACCAC | 42048255 | 42048274 |
| 2668 | GTGACGGGAAACTGACACAT | 42039564 | 42039583 | 2790 | ATTCTGAAGTCATTACCACT | 42048256 | 42048275 |
| 2669 | TGACGGGAAACTGACACATG | 42039565 | 42039584 | 2791 | TGCGATAAGTCCATGTTTGC | 42049521 | 42049540 |
| 2670 | TCAGTTGCTTGGAGTCTGTT | 42040228 | 42040247 | 2792 | GATAAGTCCATGTTTGCGGG | 42049524 | 42049543 |
| 2671 | CAGTTGCTTGGAGTCTGTTC | 42040229 | 42040248 | 2793 | ATAAGTCCATGTTTGCGGGA | 42049525 | 42049544 |
| 2672 | AGTTGCTTGGAGTCTGTTCA | 42040230 | 42040249 | 2794 | TAAGTCCATGTTTGCGGGAA | 42049526 | 42049545 |
| 2673 | GCTTGGAGTCTGTTCAGCAG | 42040234 | 42040253 | 2795 | AAGTCCATGTTTGCGGGAAC | 42049527 | 42049546 |
| 2674 | CTTGGAGTCTGTTCAGCAGC | 42040235 | 42040254 | 2796 | AGTCCATGTTTGCGGGAACC | 42049528 | 42049547 |
| 2675 | TCTATGTTCTGAGTTGATGT | 42040310 | 42040329 | 2797 | GTCCATGTTTGCGGGAACCT | 42049529 | 42049548 |
| 2676 | CTATGTTCTGAGTTGATGTC | 42040311 | 42040330 | 2798 | TCCATGTTTGCGGGAACCTA | 42049530 | 42049549 |
| 2677 | TATGTTCTGAGTTGATGTCT | 42040312 | 42040331 | 2799 | CCATGTTTGCGGGAACCTAA | 42049531 | 42049550 |
| 2678 | ATGTTCTGAGTTGATGTCTT | 42040313 | 42040332 | 2800 | CATGTTTGCGGGAACCTAAG | 42049532 | 42049551 |
| 2679 | TGTTCTGAGTTGATGTCTTG | 42040314 | 42040333 | 2801 | AATAATCATTCTTTGTCCCG | 42051912 | 42051931 |
| 2680 | GTTCTGAGTTGATGTCTTGA | 42040315 | 42040334 | 2802 | ATAATCATTCTTTGTCCCGA | 42051913 | 42051932 |
| 2681 | ATCCATGAGTGTGATGTTTA | 42040792 | 42040811 | 2803 | AATCATTCTTTGTCCCGATG | 42051915 | 42051934 |
| 2682 | TCCATGAGTGTGATGTTTAG | 42040793 | 42040812 | 2804 | AGATGATGTGATCGCCCTTG | 42052026 | 42052045 |
| 2683 | CCATGAGTGTGATGTTTAGG | 42040794 | 42040813 | 2805 | GATGATGTGATCGCCCTTGG | 42052027 | 42052046 |
| 2684 | CATGAGTGTGATGTTTAGGC | 42040795 | 42040814 | 2806 | CATTCCTGAGTTACATTACA | 42052466 | 42052485 |
| 2685 | ATGAGTGTGATGTTTAGGCT | 42040796 | 42040815 | 2807 | TCCTGAGTTACATTACATAG | 42052469 | 42052488 |
| 2686 | TGAGTGTGATGTTTAGGCTG | 42040797 | 42040816 | 2808 | CCTGAGTTACATTACATAGA | 42052470 | 42052489 |
| 2687 | GAGTGTGATGTTTAGGCTGA | 42040798 | 42040817 | 2809 | TACAGTGTTATGTGCAAATG | 42052834 | 42052853 |
| 2688 | AGTGTGATGTTTAGGCTGAG | 42040799 | 42040818 | 2810 | AAATTCAACATCATCTTCCG | 42053491 | 42053510 |
| 2689 | GTGTGATGTTTAGGCTGAGA | 42040800 | 42040819 | 2811 | AATTCAACATCATCTTCCGC | 42053492 | 42053511 |
| 2690 | TGTGATGTTTAGGCTGAGAG | 42040801 | 42040820 | 2812 | AACATCATCTTCCGCTGTGT | 42053497 | 42053516 |
| 2691 | GTGATGTTTAGGCTGAGAGT | 42040802 | 42040821 | 2813 | TCCATCCATCTGAAGCCTGT | 42053518 | 42053537 |
| 2692 | TGATGTTTAGGCTGAGAGTA | 42040803 | 42040822 | 2814 | CCATCCATCTGAAGCCTGTT | 42053519 | 42053538 |
| 2693 | GATGTTTAGGCTGAGAGTAG | 42040804 | 42040823 | 2815 | CATCCATCTGAAGCCTGTTA | 42053520 | 42053539 |
| 2694 | GCCCTGATACGACTGTTCTG | 42041420 | 42041439 | 2816 | ATCCATCTGAAGCCTGTTAG | 42053521 | 42053540 |
| 2695 | CCCTGATACGACTGTTCTGT | 42041421 | 42041440 | 2817 | TCTGAAGCCTGTTAGAATTT | 42053526 | 42053545 |
| 2696 | CCTGATACGACTGTTCTGTG | 42041422 | 42041441 | 2818 | GCAACTTGGTACGAATGAGT | 42054054 | 42054073 |
| 2697 | CTGATACGACTGTTCTGTGG | 42041423 | 42041442 | 2819 | CAACTTGGTACGAATGAGTT | 42054055 | 42054074 |
| 2698 | TGATACGACTGTTCTGTGGC | 42041424 | 42041443 | 2820 | TCCTTGGACAAACTCACTTC | 42054804 | 42054823 |
| 2699 | GATACGACTGTTCTGTGGCT | 42041425 | 42041444 | 2821 | CCTTGGACAAACTCACTTCT | 42054805 | 42054824 |
| 2700 | ATACGACTGTTCTGTGGCTC | 42041426 | 42041445 | 2822 | CTTGGACAAACTCACTTCTC | 42054806 | 42054825 |
| 2701 | TACGACTGTTCTGTGGCTCT | 42041427 | 42041446 | | | | |

Note: ASOs listed in Table 70 are designed to target IPF risk variants and IPF transplant free survival variants associated with PUFIN1, except for SEQ ID NOs: 2583-2586, 2653-2658, and 2806-2808, which target only IPF transplant free survival variants.

TABLE 71
Sequences of ASOs designed to target IPF risk variants and IPF transplant free survival variants
associated with PUFIN3.

| SEQ | | | | SEQ | | | |
|---|---|---|---|---|---|---|---|
| ID | | chr_ | chr_ | ID | | chr_ | chr_ |
| NO: | Sequence | start | end | NO: | Sequence | start | end |
| 2823 | ACAAGCCAAGTCCCAC | 38879984 | 38879999 | 2872 | GCTTAGATGGTAACAAAGTG | 38877682 | 38877701 |
| 2824 | CTGGTCTGCTACAATG | 38879838 | 38879853 | 2873 | CTTAGATGGTAACAAAGTGA | 38877681 | 38877700 |
| 2825 | GGTCTGCTACAATGAT | 38879836 | 38879851 | 2874 | CACACTGGATGAAACTGCTG | 38877576 | 38877595 |
| 2826 | GTCTGCTACAATGATC | 38879835 | 38879850 | 2875 | ACACTGGATGAAACTGCTGA | 38877575 | 38877594 |
| 2827 | TCTGCTACAATGATCC | 38879834 | 38879849 | 2876 | CACTGGATGAAACTGCTGAA | 38877574 | 38877593 |
| 2828 | AATTGCAAGTAGACCC | 38877804 | 38877819 | 2877 | ACTGGATGAAACTGCTGAAG | 38877573 | 38877592 |
| 2829 | ATTGCAAGTAGACCCA | 38877803 | 38877818 | 2878 | CTGGATGAAACTGCTGAAGG | 38877572 | 38877591 |
| 2830 | TAGGAACTGTTTAGCC | 38877760 | 38877775 | 2879 | CAGGCTGTGGTGCTAAAGGC | 38877480 | 38877499 |
| 2831 | GCATCGAGGGCATGAT | 38877709 | 38877724 | 2880 | AGGCTGTGGTGCTAAAGGCA | 38877479 | 38877498 |
| 2832 | CATCGAGGGCATGATA | 38877708 | 38877723 | 2881 | GGCTGTGGTGCTAAAGGCAT | 38877478 | 38877497 |
| 2833 | CTAGTTAAGGGTTCCA | 38874685 | 38874700 | 2882 | TGCAATCCACAGTAAGAAAT | 38876242 | 38876261 |
| 2834 | TGGGTCTAAGATAGGC | 38874245 | 38874260 | 2883 | GCAATCCACAGTAAGAAATA | 38876241 | 38876260 |
| 2835 | AACAAGCCAAGTCCCACATT | 38879981 | 38880000 | 2884 | CAGCTCATTACCTCCTACAG | 38875174 | 38875193 |
| 2836 | ACAAGCCAAGTCCCACATTG | 38879980 | 38879999 | 2885 | AGCTCATTACCTCCTACAGT | 38875173 | 38875192 |
| 2837 | GCCAAGTCCCACATTGCATC | 38879976 | 38879995 | 2886 | GCTCATTACCTCCTACAGTG | 38875172 | 38875191 |
| 2838 | CCAAGTCCCACATTGCATCA | 38879975 | 38879994 | 2887 | ATCTGATGGCTGGTCCTGGC | 38874534 | 38874553 |
| 2839 | CAAGTCCCACATTGCATCAT | 38879974 | 38879993 | 2888 | TCTGATGGCTGGTCCTGGCA | 38874533 | 38874552 |
| 2840 | AAGTCCCACATTGCATCATC | 38879973 | 38879992 | 2889 | AGGCTGGGTCTAAGATAGGC | 38874245 | 38874264 |
| 2841 | AGTCCCACATTGCATCATCC | 38879972 | 38879991 | 2890 | GGCTGGGTCTAAGATAGGCG | 38874244 | 38874263 |
| 2842 | CCACATTGCATCATCCCACA | 38879968 | 38879987 | 2891 | CATAAGGAAGTCTTAAGTTG | 38874200 | 38874219 |
| 2843 | CACATTGCATCATCCCACAT | 38879967 | 38879986 | 2892 | ATAAGGAAGTCTTAAGTTGG | 38874199 | 38874218 |
| 2844 | TTTCTGGTCTGCTACAATGA | 38879837 | 38879856 | 2893 | TAAGGAAGTCTTAAGTTGGT | 38874198 | 38874217 |
| 2845 | TTCTGGTCTGCTACAATGAT | 38879836 | 38879855 | 2894 | AAGGAAGTCTTAAGTTGGTT | 38874197 | 38874216 |
| 2846 | TCTGGTCTGCTACAATGATC | 38879835 | 38879854 | 2895 | AGGAAGTCTTAAGTTGGTTT | 38874196 | 38874215 |
| 2847 | CTGAAGACTTACTAATTTGG | 38878850 | 38878869 | 2896 | TGGCTATTTAGTGGAATTTG | 38873736 | 38873755 |
| 2848 | TGAAGACTTACTAATTTGGC | 38878849 | 38878868 | 2897 | GGCTATTTAGTGGAATTTGG | 38873735 | 38873754 |
| 2849 | CCCTGAGAATCTTAAATTGT | 38878770 | 38878789 | 2898 | GCTATTTAGTGGAATTTGGT | 38873734 | 38873753 |
| 2850 | CCTGAGAATCTTAAATTGTG | 38878769 | 38878788 | 2899 | CTATTTAGTGGAATTTGGTG | 38873733 | 38873752 |
| 2851 | GAGAATCTTAAATTGTGTGC | 38878766 | 38878785 | 2900 | TTAGTGGAATTTGGTGATTC | 38873729 | 38873748 |
| 2852 | AGAATCTTAAATTGTGTGCC | 38878765 | 38878784 | 2901 | TAGTGGAATTTGGTGATTCA | 38873728 | 38873747 |
| 2853 | GAATCTTAAATTGTGTGCCT | 38878764 | 38878783 | 2902 | AGTGGAATTTGGTGATTCAG | 38873727 | 38873746 |
| 2854 | CCTCCAGAATTGCCAAATGA | 38877922 | 38877941 | 2903 | GTGGAATTTGGTGATTCAGA | 38873726 | 38873745 |
| 2855 | TCCAGAATTGCCAAATGAGT | 38877920 | 38877939 | 2904 | TGGAATTTGGTGATTCAGAA | 38873725 | 38873744 |
| 2856 | CCAGAATTGCCAAATGAGTC | 38877919 | 38877938 | 2905 | GGAATTTGGTGATTCAGAAC | 38873724 | 38873743 |
| 2857 | CAGAATTGCCAAATGAGTCT | 38877918 | 38877937 | 2906 | GAATTTGGTGATTCAGAACT | 38873723 | 38873742 |
| 2858 | AGAATTGCCAAATGAGTCTG | 38877917 | 38877936 | 2907 | GAGACAGGGACCAAATATTT | 38873377 | 38873396 |
| 2859 | CAAATGAGTCTGGTTGTTAC | 38877909 | 38877928 | 2908 | AAACTGATGGGTGTCTGTTT | 38872769 | 38872788 |
| 2860 | AAATGAGTCTGGTTGTTACT | 38877908 | 38877927 | 2909 | GCAGCCTCCAGAATCACAAC | 38872594 | 38872613 |
| 2861 | AAATTGCAAGTAGACCCAGA | 38877801 | 38877820 | 2910 | CAGCCTCCAGAATCACAACA | 38872593 | 38872612 |
| 2862 | AATTGCAAGTAGACCCAGAA | 38877800 | 38877819 | 2911 | AGCCTCCAGAATCACAACAG | 38872592 | 38872611 |
| 2863 | ATTGCAAGTAGACCCAGAAT | 38877799 | 38877818 | 2912 | GCCTCCAGAATCACAACAGA | 38872591 | 38872610 |
| 2864 | TTGCAAGTAGACCCAGAATT | 38877798 | 38877817 | 2913 | CCTCCAGAATCACAACAGAT | 38872590 | 38872609 |
| 2865 | TGCAAGTAGACCCAGAATTA | 38877797 | 38877816 | 2914 | CTCCAGAATCACAACAGATT | 38872589 | 38872608 |
| 2866 | GAAATAGGAACTGTTTAGCC | 38877760 | 38877779 | 2915 | CAACAGATTCAGAGAGAGTC | 38872578 | 38872597 |
| 2867 | GCATCGAGGGCATGATAGAT | 38877705 | 38877724 | 2916 | GTGGCTTACTTTGGTCAATA | 38871845 | 38871864 |
| 2868 | CATCGAGGGCATGATAGATC | 38877704 | 38877723 | 2917 | TGGCTTACTTTGGTCAATAG | 38871844 | 38871863 |
| 2869 | CTGGCTTAGATGGTAACAAA | 38877685 | 38877704 | 2918 | GGCTTACTTTGGTCAATAGA | 38871843 | 38871862 |
| 2870 | TGGCTTAGATGGTAACAAAG | 38877684 | 38877703 | 2919 | GCTTACTTTGGTCAATAGAA | 38871842 | 38871861 |
| 2871 | GGCTTAGATGGTAACAAAGT | 38877683 | 38877702 | | | | |

Note: ASOs listed in Table 71 are designed to target IPF risk variants and IPF transplant free survival variants associated with PUFIN3, except for SEQ ID NO: 2833, which targets only IPF transplant free survival variants.

TABLE 72
Sequences of ASOs designed to target IPF risk variants and IPF transplant free survival variants
associated with PUFIN7.

| SEQ | | | | SEQ | | | |
|---|---|---|---|---|---|---|---|
| ID | | chr_ | chr_ | ID | | chr _ | chr_ |
| NO: | Sequence | start | end | NO: | Sequence | start | end |
| 2920 | CTGGCTATACAATGAC | 215719137 | 215719152 | 2928 | AGAATCACCACCTCACATTC | 215719728 | 215719747 |
| 2921 | GGCTATACAATGACAG | 215719139 | 215719154 | 2929 | GAATCACCACCTCACATTCT | 215719729 | 215719748 |
| 2922 | GTCCAGTAAAGAGACC | 215720454 | 215720469 | 2930 | AATCACCACCTCACATTCTA | 215719730 | 215719749 |
| 2923 | CAACAGCACCTCTTCCTTAA | 215719274 | 215719293 | 2931 | AGCAGCAGACAAGTAAGTTC | 215719976 | 215719995 |
| 2924 | AACAGCACCTCTTCCTTAAA | 215719275 | 215719294 | 2932 | GCAGCAGACAAGTAAGTTCA | 215719977 | 215719996 |
| 2925 | ATAATAGAGCAGAATCACCA | 215719718 | 215719737 | 2933 | CAGCAGACAAGTAAGTTCAG | 215719978 | 215719997 |
| 2926 | GCAGAATCACCACCTCACAT | 215719726 | 215719745 | 2934 | CTGGAGAAGCCAATAAAGTT | 215720313 | 215720332 |
| 2927 | CAGAATCACCACCTCACATT | 215719727 | 215719746 | 2935 | TGGAGAAGCCAATAAAGTTT | 215720314 | 215720333 |

Note: ASOs listed in Table 72 are designed to target IPF risk variants and IPF transplant free survival variants associated with PUFIN7, except for SEQ ID NO: 2922, which targets only IPF transplant free survival variants.

TABLE 73
Sequences of ASOs designed to target IPF risk variants and IPF transplant free survival
variants associated with PUFIN2.

| SEQ | | | | SEQ | | | |
|---|---|---|---|---|---|---|---|
| ID | | chr_ | chr_ | ID | | chr_ | chr_ |
| NO: | Sequence | start | end | NO: | Sequence | start | end |
| 2936 | GCCGTAGAGAGCAACC | 39427180 | 39427195 | 3420 | TGACTCGATGGTACTG | 39068586 | 39068601 |
| 2937 | CCGTAGAGAGCAACCA | 39427179 | 39427194 | 3421 | GACTCGATGGTACTGT | 39068585 | 39068600 |
| 2938 | TCGGTCAGATGGAAAC | 39418247 | 39418262 | 3422 | ACTCGATGGTACTGTT | 39068584 | 39068599 |
| 2939 | GTAGTGCGGTGTTAAG | 39407446 | 39407461 | 3423 | CTCGATGGTACTGTTA | 39068583 | 39068598 |
| 2940 | TAGTGCGGTGTTAAGG | 39407445 | 39407460 | 3424 | TCGATGGTACTGTTAC | 39068582 | 39068597 |
| 2941 | GCCACAAGAGACCACG | 39406674 | 39406689 | 3425 | CGATGGTACTGTTACA | 39068581 | 39068596 |
| 2942 | CCACAAGAGACCACGA | 39406673 | 39406688 | 3426 | GATCCGAAACTTAGAT | 39030764 | 39030779 |
| 2943 | ACGTTCCATGTAGTTC | 39402672 | 39402687 | 3427 | ATCCGAAACTTAGATG | 39030763 | 39030778 |
| 2944 | CATGTAGTTCCGTGGA | 39402666 | 39402681 | 3428 | GTACGAATGGAACTCA | 39064162 | 39064177 |
| 2945 | ATGTAGTTCCGTGGAT | 39402665 | 39402680 | 3429 | TACGAATGGAACTCAA | 39064161 | 39064176 |
| 2946 | TGTAGTTCCGTGGATG | 39402664 | 39402679 | 3430 | ACGAATGGAACTCAAT | 39064160 | 39064175 |
| 2947 | GTAGTTCCGTGGATGC | 39402663 | 39402678 | 3431 | CGAATGGAACTCAATG | 39064159 | 39064174 |
| 2948 | TAGTTCCGTGGATGCC | 39402662 | 39402677 | 3432 | TCCGAAACTTAGATGC | 39030762 | 39030777 |
| 2949 | AGTTCCGTGGATGCCC | 39402661 | 39402676 | 3433 | CCGAAACTTAGATGCA | 39030761 | 39030776 |
| 2950 | GGTTCCACCAGACGAT | 39402448 | 39402463 | 3434 | CGAAACTTAGATGCAG | 39030760 | 39030775 |
| 2951 | ACCAGACGATGCCAGA | 39402442 | 39402457 | 3435 | GTAAGTTTCTGGAGCG | 39027810 | 39027825 |
| 2952 | CCAGACGATGCCAGAA | 39402441 | 39402456 | 3436 | GCGCTTGGAATTGATT | 39027797 | 39027812 |
| 2953 | CAGACGATGCCAGAAT | 39402440 | 39402455 | 3437 | CGCTTGGAATTGATTC | 39027796 | 39027811 |
| 2954 | GACGATGCCAGAATAT | 39402438 | 39402453 | 3438 | CAATATCGCTTCCATA | 39023281 | 39023296 |
| 2955 | ACGATGCCAGAATATC | 39402437 | 39402452 | 3439 | ACTGAAGATACGGATC | 39054265 | 39054280 |
| 2956 | CGATGCCAGAATATCT | 39402436 | 39402451 | 3440 | AATATCGCTTCCATAG | 39023280 | 39023295 |
| 2957 | GCGTCTCTGCTGATTG | 39395200 | 39395215 | 3441 | GCCATCATACTGTCCG | 39023155 | 39023170 |
| 2958 | TTTGACAGTTAGCGTG | 39391474 | 39391489 | 3442 | CATCATACTGTCCGTG | 39023153 | 39023168 |
| 2959 | TTGACAGTTAGCGTGC | 39391473 | 39391488 | 3443 | ATCATACTGTCCGTGA | 39023152 | 39023167 |
| 2960 | CGTGCCATGCTTTGGA | 39391462 | 39391477 | 3444 | TCATACTGTCCGTGAG | 39023151 | 39023166 |
| 2961 | AGGAAGTGATCTTACG | 39389642 | 39389657 | 3445 | CATACTGTCCGTGAGC | 39023150 | 39023165 |
| 2962 | GGAAGTGATCTTACGA | 39389641 | 39389656 | 3446 | ATACTGTCCGTGAGCC | 39023149 | 39023164 |
| 2963 | GAAGTGATCTTACGAC | 39389640 | 39389655 | 3447 | TACTGTCCGTGAGCCA | 39023148 | 39023163 |
| 2964 | ACTGTCGTGTAGTTGT | 39389124 | 39389139 | 3448 | ACTGTCCGTGAGCCAT | 39023147 | 39023162 |
| 2965 | TGTCGTGTAGTTGTGC | 39389122 | 39389137 | 3449 | CTGTCCGTGAGCCATC | 39023146 | 39023161 |
| 2966 | GTCGTGTAGTTGTGCT | 39389121 | 39389136 | 3450 | GCAGGCATTGTGACGG | 39019046 | 39019061 |
| 2967 | TCGTGTAGTTGTGCTA | 39389120 | 39389135 | 3451 | GCATTGTGACGGAAGT | 39019042 | 39019057 |
| 2968 | TTTCAAAGAGGGACCG | 39385525 | 39385540 | 3452 | CATTGTGACGGAAGTA | 39019041 | 39019056 |
| 2969 | TTCAAAGAGGGACCGT | 39385524 | 39385539 | 3453 | TAATTACAGGAGTACG | 39014975 | 39014990 |
| 2970 | TCAAAGAGGGACCGTT | 39385523 | 39385538 | 3454 | ATTATAATCTGGGACG | 39013603 | 39013618 |
| 2971 | CACTCTCTCTAACGAA | 39379548 | 39379563 | 3455 | TTATAATCTGGGACGT | 39013602 | 39013617 |
| 2972 | ACTCTCTCTAACGAAG | 39379547 | 39379562 | 3456 | AGGTAGGTGACGGTTC | 39013082 | 39013097 |
| 2973 | TCTCTCTAACGAAGCC | 39379545 | 39379560 | 3457 | GGTAGGTGACGGTTCT | 39013081 | 39013096 |
| 2974 | CTCTCTAACGAAGCCA | 39379544 | 39379559 | 3458 | GTAGGTGACGGTTCTT | 39013080 | 39013095 |
| 2975 | TCCGATTGTTGATTGC | 39374758 | 39374773 | 3459 | TAGGTGACGGTTCTTC | 39013079 | 39013094 |
| 2976 | GATTGTTGATTGCGGT | 39374755 | 39374770 | 3460 | AGGTGACGGTTCTTCC | 39013078 | 39013093 |
| 2977 | ATTGTTGATTGCGGTG | 39374754 | 39374769 | 3461 | GGTGACGGTTCTTCCT | 39013077 | 39013092 |
| 2978 | TTGTTGATTGCGGTGT | 39374753 | 39374768 | 3462 | GCCGTATCTAGTCCTG | 39011917 | 39011932 |
| 2979 | TGTTGATTGCGGTGTC | 39374752 | 39374767 | 3463 | CACGTAGAAATTTAGC | 39008052 | 39008067 |
| 2980 | GTTGATTGCGGTGTCC | 39374751 | 39374766 | 3464 | GGGACCACCACAATAG | 39412710 | 39412725 |
| 2981 | TTGATTGCGGTGTCCT | 39374750 | 39374765 | 3465 | GGACCACCACAATAGA | 39412709 | 39412724 |
| 2982 | TGATTGCGGTGTCCTA | 39374749 | 39374764 | 3466 | GATCCTTAACTATCAC | 39412675 | 39412690 |
| 2983 | GATTGCGGTGTCCTAA | 39374748 | 39374763 | 3467 | AAGCTCGTTGTCTTGG | 38995484 | 38995499 |
| 2984 | ATTGCGGTGTCCTAAA | 39374747 | 39374762 | 3468 | AGCTCGTTGTCTTGGA | 38995483 | 38995498 |
| 2985 | TTGCGGTGTCCTAAAT | 39374746 | 39374761 | 3469 | GCTCGTTGTCTTGGAA | 38995482 | 38995497 |
| 2986 | TGCGGTGTCCTAAATA | 39374745 | 39374760 | 3470 | CTCGTTGTCTTGGAAC | 38995481 | 38995496 |
| 2987 | GCGGTGTCCTAAATAC | 39374744 | 39374759 | 3471 | TCGTTGTCTTGGAACT | 38995480 | 38995495 |
| 2988 | CGGTGTCCTAAATACT | 39374743 | 39374758 | 3472 | CGTTGTCTTGGAACTG | 38995479 | 38995494 |
| 2989 | ACGTGTCTGATCCCAA | 39363900 | 39363915 | 3473 | CCATCTAGTAGCTCGC | 39018857 | 39018872 |
| 2990 | CGTGTCTGATCCCAAA | 39363899 | 39363914 | 3474 | GCTCGCTTATGATCTA | 39018847 | 39018862 |
| 2991 | CTATAGCGGGATATGG | 39363718 | 39363733 | 3475 | TCGCTTATGATCTATG | 39018845 | 39018860 |
| 2992 | TAGCACACTGAAAGCG | 39362351 | 39362366 | 3476 | CGCTTATGATCTATGA | 39018844 | 39018859 |
| 2993 | ACTGAAAGCGTCAATA | 39362345 | 39362360 | 3477 | CTATAGCGTGACAGGA | 38994334 | 38994349 |
| 2994 | CGATCCTTGGCATGAC | 39348846 | 39348861 | 3478 | TAGCTGGTATCGTAAA | 39014044 | 39014059 |
| 2995 | TGTAAGTCTTATCACG | 39348305 | 39348320 | 3479 | AGCTGGTATCGTAAAG | 39014043 | 39014058 |
| 2996 | GTAAGTCTTATCACGT | 39348304 | 39348319 | 3480 | TATAGCGTGACAGGAT | 38994333 | 38994348 |
| 2997 | TAAGTCTTATCACGTC | 39348303 | 39348318 | 3481 | ATAGCGTGACAGGATT | 38994332 | 38994347 |
| 2998 | AAGTCTTATCACGTCC | 39348302 | 39348317 | 3482 | TAGCGTGACAGGATTG | 38994331 | 38994346 |
| 2999 | GACATTACGAGAGATT | 39345320 | 39345335 | 3483 | GCGTGACAGGATTGGT | 38994329 | 38994344 |
| 3000 | CTGAACAGACACACGC | 39341699 | 39341714 | 3484 | CGTGACAGGATTGGTA | 38994328 | 38994343 |
| 3001 | AACTCTAGTCGGTTTC | 39336196 | 39336211 | 3485 | GACAACATTTGGCTCG | 38993477 | 38993492 |
| 3002 | TATTCAAGTTTACGGG | 39336105 | 39336120 | 3486 | ACAACATTTGGCTCGC | 38993476 | 38993491 |
| 3003 | ATTCAAGTTTACGGGC | 39336104 | 39336119 | 3487 | CTCGGTTGGTGAGCAT | 38993401 | 38993416 |
| 3004 | TTCAAGTTTACGGGCT | 39336103 | 39336118 | 3488 | TCGGTTGGTGAGCATG | 38993400 | 38993415 |
| 3005 | TCAAGTTTACGGGCTA | 39336102 | 39336117 | 3489 | CATAACTGCATTCGCT | 38992559 | 38992574 |
| 3006 | CAAGTTTACGGGCTAG | 39336101 | 39336116 | 3490 | ATAACTGCATTCGCTA | 38992558 | 38992573 |
| 3007 | AAGTTTACGGGCTAGG | 39336100 | 39336115 | 3491 | TAACTGCATTCGCTAA | 38992557 | 38992572 |
| 3008 | GTAAACAAAGGCTCGC | 39358369 | 39358384 | 3492 | AACTGCATTCGCTAAA | 38992556 | 38992571 |
| 3009 | TAAACAAAGGCTCGCT | 39358368 | 39358383 | 3493 | ACTGCATTCGCTAAAT | 38992555 | 38992570 |
| 3010 | AACAAAGGCTCGCTTC | 39358366 | 39358381 | 3494 | CTGCATTCGCTAAATT | 38992554 | 38992569 |
| 3011 | ACAAAGGCTCGCTTCA | 39358365 | 39358380 | 3495 | TGCATTCGCTAAATTG | 38992553 | 38992568 |
| 3012 | CAAAGGCTCGCTTCAT | 39358364 | 39358379 | 3496 | GCATTCGCTAAATTGA | 38992552 | 38992567 |
| 3013 | AAAGGCTCGCTTCATG | 39358363 | 39358378 | 3497 | TAGCCTACGGTTTGAA | 38990559 | 38990574 |
| 3014 | AGTTTACGGGCTAGGC | 39336099 | 39336114 | 3498 | AGCCTACGGTTTGAAT | 38990558 | 38990573 |
| 3015 | TCGGCTCAATCTGTAC | 39333418 | 39333433 | 3499 | GCCTACGGTTTGAATC | 38990557 | 38990572 |
| 3016 | CCGGTCTGGATGTCTG | 39332986 | 39333001 | 3500 | CCTACGGTTTGAATCA | 38990556 | 38990571 |
| 3017 | CCGCTCAGGCAAGGTT | 39331295 | 39331310 | 3501 | CTACGGTTTGAATCAT | 38990555 | 38990570 |
| 3018 | CGCTCAGGCAAGGTTA | 39331294 | 39331309 | 3502 | CGGTTTGAATCATATC | 38990552 | 38990567 |
| 3019 | CCACACGAGAACTAAG | 39350678 | 39350693 | 3503 | AAGCAACATTTATCCG | 38988595 | 38988610 |
| 3020 | CACACGAGAACTAAGC | 39350677 | 39350692 | 3504 | AGCAACATTTATCCGT | 38988594 | 38988609 |
| 3021 | GAGAACTTCACACGAT | 39330264 | 39330279 | 3505 | GCAACATTTATCCGTG | 38988593 | 38988608 |
| 3022 | AGAACTTCACACGATG | 39330263 | 39330278 | 3506 | CAACATTTATCCGTGC | 38988592 | 38988607 |
| 3023 | GTTGTCACATTAACCG | 39328401 | 39328416 | 3507 | AACATTTATCCGTGCA | 38988591 | 38988606 |
| 3024 | TTGTCACATTAACCGT | 39328400 | 39328415 | 3508 | CATTTATCCGTGCATA | 38988589 | 38988604 |
| 3025 | TGTCACATTAACCGTC | 39328399 | 39328414 | 3509 | AAACCACCAATCTTCG | 38986301 | 38986316 |
| 3026 | AACTACTGTGTGTACG | 39326684 | 39326699 | 3510 | CAATCTTCGGCTGGGC | 38986294 | 38986309 |
| 3027 | ACTACTGTGTGTACGG | 39326683 | 39326698 | 3511 | GATATCCTTACTCACG | 38985186 | 38985201 |
| 3028 | CTACTGTGTGTACGGT | 39326682 | 39326697 | 3512 | ATATCCTTACTCACGA | 38985185 | 38985200 |
| 3029 | AACTCAGACGTGGCTT | 39339047 | 39339062 | 3513 | ATCCTTACTCACGATA | 38985183 | 38985198 |
| 3030 | ACTCAGACGTGGCTTC | 39339046 | 39339061 | 3514 | TCCTTACTCACGATAA | 38985182 | 38985197 |
| 3031 | AGACGTGGCTTCAGAC | 39339042 | 39339057 | 3515 | CCTTACTCACGATAAA | 38985181 | 38985196 |
| 3032 | GACGTGGCTTCAGACT | 39339041 | 39339056 | 3516 | TAAATATTCCGAGACC | 38985169 | 38985184 |
| 3033 | TACTGTGTGTACGGTG | 39326681 | 39326696 | 3517 | ACAGCGTGATAAATTG | 38981489 | 38981504 |
| 3034 | GTGTACGGTGGTGGAC | 39326675 | 39326690 | 3518 | CAGCGTGATAAATTGG | 38981488 | 38981503 |
| 3035 | TGTACGGTGGTGGACA | 39326674 | 39326689 | 3519 | GCGTGATAAATTGGAT | 38981486 | 38981501 |
| 3036 | GTACGGTGGTGGACAT | 39326673 | 39326688 | 3520 | GCGTGGTCATTACATA | 38975693 | 38975708 |
| 3037 | TACGGTGGTGGACATT | 39326672 | 39326687 | 3521 | CGTGGTCATTACATAG | 38975692 | 38975707 |
| 3038 | ACGGTGGTGGACATTA | 39326671 | 39326686 | 3522 | TGTTTGAAAGCCACGC | 38974522 | 38974537 |
| 3039 | CGGGTACTATTGGCAA | 39319704 | 39319719 | 3523 | CGCCTCTATGGAAATC | 38974509 | 38974524 |
| 3040 | GCAATATTCCTCGTGC | 39312088 | 39312103 | 3524 | TCACACCAATTCACGT | 38973845 | 38973860 |
| 3041 | GCGCCACATAAGATGA | 39311925 | 39311940 | 3525 | CACACCAATTCACGTT | 38973844 | 38973859 |
| 3042 | CGCCACATAAGATGAA | 39311924 | 39311939 | 3526 | ACACCAATTCACGTTG | 38973843 | 38973858 |
| 3043 | TAAATTCCGTTTGAGC | 39332791 | 39332806 | 3527 | CAATTCACGTTGGACA | 38973839 | 38973854 |
| 3044 | AATTCCGTTTGAGCAC | 39332789 | 39332804 | 3528 | ACACACGAATGAGCTT | 38969954 | 38969969 |
| 3045 | ATTCCGTTTGAGCACC | 39332788 | 39332803 | 3529 | CACACGAATGAGCTTC | 38969953 | 38969968 |
| 3046 | CCGTTTGAGCACCTTG | 39332785 | 39332800 | 3530 | ACACGAATGAGCTTCA | 38969952 | 38969967 |
| 3047 | CGTTTGAGCACCTTGT | 39332784 | 39332799 | 3531 | CACGAATGAGCTTCAA | 38969951 | 38969966 |
| 3048 | GAATTGAGGCTTAACG | 39296306 | 39296321 | 3532 | ACGAATGAGCTTCAAA | 38969950 | 38969965 |
| 3049 | AATTGAGGCTTAACGT | 39296305 | 39296320 | 3533 | CGAATGAGCTTCAAAT | 38969949 | 38969964 |
| 3050 | ATTGAGGCTTAACGTT | 39296304 | 39296319 | 3534 | TCGATAATGAGTAAGC | 38967203 | 38967218 |
| 3051 | TGAGGCTTAACGTTTG | 39296302 | 39296317 | 3535 | CGATAATGAGTAAGCC | 38967202 | 38967217 |
| 3052 | GCTTAACGTTTGCATA | 39296298 | 39296313 | 3536 | AGTCCTCGTGAATGGC | 38965661 | 38965676 |
| 3053 | CCTAAATCCGATGATT | 39296116 | 39296131 | 3537 | GTCCTCGTGAATGGCA | 38965660 | 38965675 |
| 3054 | CTAAATCCGATGATTG | 39296115 | 39296130 | 3538 | TTTGAAAGCGTCATGC | 38964498 | 38964513 |
| 3055 | TAAATCCGATGATTGG | 39296114 | 39296129 | 3539 | AATTTCGTATGGTCAG | 38956231 | 38956246 |
| 3056 | AAATCCGATGATTGGT | 39296113 | 39296128 | 3540 | CCTGCAAACACACCGA | 38980294 | 38980309 |
| 3057 | AATCCGATGATTGGTC | 39296112 | 39296127 | 3541 | CTGCAAACACACCGAC | 38980293 | 38980308 |
| 3058 | CGATGATTGGTCTCCT | 39296108 | 39296123 | 3542 | TGCAAACACACCGACT | 38980292 | 38980307 |
| 3059 | AGAACCTATTCCGTAT | 39285617 | 39285632 | 3543 | GCAAACACACCGACTC | 38980291 | 38980306 |
| 3060 | AACCTATTCCGTATGG | 39285615 | 39285630 | 3544 | CAAACACACCGACTCC | 38980290 | 38980305 |
| 3061 | GGTCTTCCCACACGAA | 39283015 | 39283030 | 3545 | AAACACACCGACTCCC | 38980289 | 38980304 |
| 3062 | CTTCCCACACGAAATG | 39283012 | 39283027 | 3546 | AACACACCGACTCCCA | 38980288 | 38980303 |
| 3063 | CACACGAAATGGCTGT | 39283007 | 39283022 | 3547 | TTTCGTATGGTCAGTC | 38956229 | 38956244 |
| 3064 | CCATTTCAGCGGATTA | 39322809 | 39322824 | 3548 | ACGCAGTCTTAAGTTC | 38951268 | 38951283 |
| 3065 | CATTTCAGCGGATTAT | 39322808 | 39322823 | 3549 | CGCAGTCTTAAGTTCT | 38951267 | 38951282 |
| 3066 | TTCAGCGGATTATATC | 39322805 | 39322820 | 3550 | TGCTCACTGCCCGATG | 38942102 | 38942117 |
| 3067 | ACACGAAATGGCTGTC | 39283006 | 39283021 | 3551 | CTTAACTACCCACGAG | 38941578 | 38941593 |
| 3068 | CTGTCTTGTAAGAACG | 39315312 | 39315327 | 3552 | CACCTGGGACAAACCG | 38941250 | 38941265 |
| 3069 | TGTCTTGTAAGAACGG | 39315311 | 39315326 | 3553 | ACCTGGGACAAACCGC | 38941249 | 38941264 |
| 3070 | GTCTTGTAAGAACGGG | 39315310 | 39315325 | 3554 | CCTGGGACAAACCGCT | 38941248 | 38941263 |
| 3071 | TCTTGTAAGAACGGGC | 39315309 | 39315324 | 3555 | CTGGGACAAACCGCTT | 38941247 | 38941262 |
| 3072 | CTTGTAAGAACGGGCT | 39315308 | 39315323 | 3556 | ACAGATTAGATGTACG | 38940629 | 38940644 |
| 3073 | TTGTAAGAACGGGCTA | 39315307 | 39315322 | 3557 | CAGATTAGATGTACGC | 38940628 | 38940643 |
| 3074 | AGAACGGGCTAGACTT | 39315302 | 39315317 | 3558 | TCCGTATTCTTGTAAG | 38937064 | 38937079 |
| 3075 | GAACGGGCTAGACTTA | 39315301 | 39315316 | 3559 | CCGTATTCTTGTAAGG | 38937063 | 38937078 |
| 3076 | AACGGGCTAGACTTAG | 39315300 | 39315315 | 3560 | CGTATTCTTGTAAGGA | 38937062 | 38937077 |
| 3077 | ACGGGCTAGACTTAGT | 39315299 | 39315314 | 3561 | AGTCAAAGTCGAAGTT | 38936257 | 38936272 |
| 3078 | CGGGCTAGACTTAGTG | 39315298 | 39315313 | 3562 | CAAAGTCGAAGTTGGT | 38936254 | 38936269 |
| 3079 | CACGAAATGGCTGTCT | 39283005 | 39283020 | 3563 | TTACGCCAGTGTTCTA | 38935340 | 38935355 |
| 3080 | CGAAATGGCTGTCTTG | 39283003 | 39283018 | 3564 | TACGCCAGTGTTCTAA | 38935339 | 38935354 |
| 3081 | TGTCCTTACCGAAATG | 39278729 | 39278744 | 3565 | ACGCCAGTGTTCTAAA | 38935338 | 38935353 |
| 3082 | GTCCTTACCGAAATGT | 39278728 | 39278743 | 3566 | CGCCAGTGTTCTAAAG | 38935337 | 38935352 |
| 3083 | TCCTTACCGAAATGTC | 39278727 | 39278742 | 3567 | GCATGATATTCTACGG | 38934604 | 38934619 |
| 3084 | CCTTACCGAAATGTCA | 39278726 | 39278741 | 3568 | CATGATATTCTACGGT | 38934603 | 38934618 |
| 3085 | GTCGTATAATTTGCTG | 39277850 | 39277865 | 3569 | ATGATATTCTACGGTG | 38934602 | 38934617 |
| 3086 | TCGTATAATTTGCTGC | 39277849 | 39277864 | 3570 | TGATATTCTACGGTGT | 38934601 | 38934616 |
| 3087 | CGTATAATTTGCTGCT | 39277848 | 39277863 | 3571 | GATATTCTACGGTGTA | 38934600 | 38934615 |
| 3088 | GTAGATATGGATGTCG | 39273988 | 39274003 | 3572 | CAATTCTACGAAGGTT | 38933162 | 38933177 |
| 3089 | TAGATATGGATGTCGT | 39273987 | 39274002 | 3573 | TGGTCCTAGCAGATCG | 38932132 | 38932147 |
| 3090 | AGATATGGATGTCGTC | 39273986 | 39274001 | 3574 | GGTCCTAGCAGATCGG | 38932131 | 38932146 |
| 3091 | GATATGGATGTCGTCA | 39273985 | 39274000 | 3575 | CCTAGCAGATCGGAAA | 38932128 | 38932143 |
| 3092 | ATATGGATGTCGTCAC | 39273984 | 39273999 | 3576 | CCCTTGCGAGATACTA | 38930933 | 38930948 |
| 3093 | TATGGATGTCGTCACA | 39273983 | 39273998 | 3577 | CCTTGCGAGATACTAC | 38930932 | 38930947 |
| 3094 | ATGGATGTCGTCACAT | 39273982 | 39273997 | 3578 | AGCATCTACGGAACAC | 38929616 | 38929631 |
| 3095 | TCTAGGTCACATTCGT | 39266745 | 39266760 | 3579 | GCATCTACGGAACACA | 38929615 | 38929630 |
| 3096 | CTAGGTCACATTCGTT | 39266744 | 39266759 | 3580 | CATCTACGGAACACAG | 38929614 | 38929629 |
| 3097 | TAGGTCACATTCGTTG | 39266743 | 39266758 | 3581 | ATCTACGGAACACAGC | 38929613 | 38929628 |
| 3098 | AGGTCACATTCGTTGA | 39266742 | 39266757 | 3582 | TCTACGGAACACAGCT | 38929612 | 38929627 |
| 3099 | GGTCACATTCGTTGAT | 39266741 | 39266756 | 3583 | CTACGGAACACAGCTA | 38929611 | 38929626 |
| 3100 | TCATCGAAGAGGTTGC | 39264591 | 39264606 | 3584 | TACGGAACACAGCTAA | 38929610 | 38929625 |
| 3101 | CATCGAAGAGGTTGCA | 39264590 | 39264605 | 3585 | ATCTAGTCTGGGTCCG | 38924230 | 38924245 |
| 3102 | AGCTCCGTCATGTGAT | 39252302 | 39252317 | 3586 | TCTAGTCTGGGTCCGT | 38924229 | 38924244 |
| 3103 | GCTCCGTCATGTGATG | 39252301 | 39252316 | 3587 | TAACAAGTCAGATTCG | 38935257 | 38935272 |
| 3104 | CTCCGTCATGTGATGA | 39252300 | 39252315 | 3588 | AACAAGTCAGATTCGT | 38935256 | 38935271 |
| 3105 | TCCGTCATGTGATGAG | 39252299 | 39252314 | 3589 | AGTCAGATTCGTCACT | 38935252 | 38935267 |
| 3106 | CCGTCATGTGATGAGT | 39252298 | 39252313 | 3590 | GTCAGATTCGTCACTT | 38935251 | 38935266 |
| 3107 | CGTCATGTGATGAGTG | 39252297 | 39252312 | 3591 | CAGATTCGTCACTTGT | 38935249 | 38935264 |
| 3108 | TTTGCACGAGTTCATC | 39274501 | 39274516 | 3592 | AGATTCGTCACTTGTT | 38935248 | 38935263 |
| 3109 | TTGCACGAGTTCATCA | 39274500 | 39274515 | 3593 | CTAGTCTGGGTCCGTA | 38924228 | 38924243 |
| 3110 | TGCACGAGTTCATCAG | 39274499 | 39274514 | 3594 | TCTGGGTCCGTATTTA | 38924224 | 38924239 |
| 3111 | GCACGAGTTCATCAGG | 39274498 | 39274513 | 3595 | CTGGGTCCGTATTTAT | 38924223 | 38924238 |
| 3112 | CACGAGTTCATCAGGT | 39274497 | 39274512 | 3596 | TGGGTCCGTATTTATC | 38924222 | 38924237 |
| 3113 | ACGAGTTCATCAGGTC | 39274496 | 39274511 | 3597 | GGGTCCGTATTTATCT | 38924221 | 38924236 |
| 3114 | CGAGTTCATCAGGTCA | 39274495 | 39274510 | 3598 | ACTCAGGCTTGGCGAA | 38920957 | 38920972 |
| 3115 | AAAGTTTGCGAATGGC | 39249852 | 39249867 | 3599 | CTCAGGCTTGGCGAAT | 38920956 | 38920971 |
| 3116 | AAGTTTGCGAATGGCT | 39249851 | 39249866 | 3600 | TCAGGCTTGGCGAATC | 38920955 | 38920970 |
| 3117 | AGTTTGCGAATGGCTG | 39249850 | 39249865 | 3601 | AAACATAAGTCGCCTA | 38919024 | 38919039 |
| 3118 | GTTTGCGAATGGCTGA | 39249849 | 39249864 | 3602 | ATTTCTTCGGTAGAGG | 38917374 | 38917389 |
| 3119 | TTTGCGAATGGCTGAG | 39249848 | 39249863 | 3603 | TTTCTTCGGTAGAGGA | 38917373 | 38917388 |
| 3120 | GCGCTAATTCTGTGAC | 39249318 | 39249333 | 3604 | ATCGATTACTAGGGTA | 38916346 | 38916361 |
| 3121 | CGCTAATTCTGTGACT | 39249317 | 39249332 | 3605 | TCGATTACTAGGGTAG | 38916345 | 38916360 |
| 3122 | CGGTCCATATTTGAAT | 39248369 | 39248384 | 3606 | CGATTACTAGGGTAGA | 38916344 | 38916359 |
| 3123 | TTTCCGCCAAGTGTGG | 39243164 | 39243179 | 3607 | CCTTCCAGACAAACCG | 38910294 | 38910309 |
| 3124 | TGTGATTTGTCATACG | 39230856 | 39230871 | 3608 | CCAGACAAACCGAAGC | 38910290 | 38910305 |
| 3125 | GTGATTTGTCATACGT | 39230855 | 39230870 | 3609 | AACATCAGCGTTAAAC | 38909223 | 38909238 |
| 3126 | TGATTTGTCATACGTG | 39230854 | 39230869 | 3610 | ACATCAGCGTTAAACA | 38909222 | 38909237 |
| 3127 | CACGTAAGTACCAAAT | 39266169 | 39266184 | 3611 | CATCAGCGTTAAACAA | 38909221 | 38909236 |
| 3128 | TTGTCATACGTGGATG | 39230850 | 39230865 | 3612 | ATCAGCGTTAAACAAC | 38909220 | 38909235 |
| 3129 | GTCATACGTGGATGGA | 39230848 | 39230863 | 3613 | TCAGCGTTAAACAACA | 38909219 | 38909234 |
| 3130 | TCATACGTGGATGGAT | 39230847 | 39230862 | 3614 | CACTTAAAGGCTGCGT | 38907380 | 38907395 |
| 3131 | TACGTGGATGGATGTC | 39230844 | 39230859 | 3615 | ACTTAAAGGCTGCGTA | 38907379 | 38907394 |
| 3132 | ACGTGGATGGATGTCA | 39230843 | 39230858 | 3616 | CTTAAAGGCTGCGTAA | 38907378 | 38907393 |
| 3133 | CTCCATAACCTCGAAT | 39226039 | 39226054 | 3617 | CTGGTAGTAAATGCCG | 38904461 | 38904476 |
| 3134 | TCCATAACCTCGAATT | 39226038 | 39226053 | 3618 | TGGTAGTAAATGCCGT | 38904460 | 38904475 |
| 3135 | CCAAACACCTTTCGCA | 39225837 | 39225852 | 3619 | GGTAGTAAATGCCGTG | 38904459 | 38904474 |
| 3136 | CAAACACCTTTCGCAT | 39225836 | 39225851 | 3620 | GTAGTAAATGCCGTGA | 38904458 | 38904473 |
| 3137 | ACACCTTTCGCATTGG | 39225833 | 39225848 | 3621 | TAGTAAATGCCGTGAC | 38904457 | 38904472 |
| 3138 | ACCTTTCGCATTGGAA | 39225831 | 39225846 | 3622 | CGGTTTCTGAGATATA | 38902837 | 38902852 |
| 3139 | CCTTTCGCATTGGAAC | 39225830 | 39225845 | 3623 | TAGGCGTATCTTTCTG | 38901031 | 38901046 |
| 3140 | CTTTCGCATTGGAACA | 39225829 | 39225844 | 3624 | AGGCGTATCTTTCTGC | 38901030 | 38901045 |
| 3141 | TTTCGCATTGGAACAG | 39225828 | 39225843 | 3625 | GGCGTATCTTTCTGCA | 38901029 | 38901044 |
| 3142 | TCGCATTGGAACAGGA | 39225826 | 39225841 | 3626 | GGTGTTGTCCGCATGG | 38897475 | 38897490 |
| 3143 | ATAACGGAATTGCATG | 39216707 | 39216722 | 3627 | CAGGGTAGATTACTCG | 38889601 | 38889616 |
| 3144 | TAACGGAATTGCATGA | 39216706 | 39216721 | 3628 | AGGGTAGATTACTCGT | 38889600 | 38889615 |
| 3145 | AACGGAATTGCATGAT | 39216705 | 39216720 | 3629 | GGGTAGATTACTCGTT | 38889599 | 38889614 |
| 3146 | ACGGAATTGCATGATT | 39216704 | 39216719 | 3630 | GGTAGATTACTCGTTG | 38889598 | 38889613 |
| 3147 | CGGAATTGCATGATTT | 39216703 | 39216718 | 3631 | GTAGATTACTCGTTGT | 38889597 | 38889612 |
| 3148 | CCGCTGTGTCTATGAG | 39211930 | 39211945 | 3632 | TAGATTACTCGTTGTG | 38889596 | 38889611 |
| 3149 | CGCTGTGTCTATGAGA | 39211929 | 39211944 | 3633 | AGATTACTCGTTGTGC | 38889595 | 38889610 |
| 3150 | GTTCAGAGACACGGTG | 39208776 | 39208791 | 3634 | GATTACTCGTTGTGCA | 38889594 | 38889609 |
| 3151 | AGACACGGTGGGATCT | 39208770 | 39208785 | 3635 | ATTACTCGTTGTGCAT | 38889593 | 38889608 |
| 3152 | GACACGGTGGGATCTA | 39208769 | 39208784 | 3636 | ACTCGTTGTGCATTTG | 38889590 | 38889605 |
| 3153 | ACACGGTGGGATCTAT | 39208768 | 39208783 | 3637 | ACGATTGTAGAAAGCC | 38883204 | 38883219 |
| 3154 | CACGGTGGGATCTATT | 39208767 | 39208782 | 3638 | CGATTGTAGAAAGCCA | 38883203 | 38883218 |
| 3155 | ACGGTGGGATCTATTT | 39208766 | 39208781 | 3639 | TTATTGGGTGAAGGCG | 38882173 | 38882188 |
| 3156 | CGGTGGGATCTATTTG | 39208765 | 39208780 | 3640 | GCATCGAGGGCATGAT | 38877709 | 38877724 |
| 3157 | ACGGCATTGGTTGTTT | 39205793 | 39205808 | 3641 | CATCGAGGGCATGATA | 38877708 | 38877723 |
| 3158 | GGACTTAAACATTCGT | 39202781 | 39202796 | 3642 | CATATGGACGATGCCT | 38870705 | 38870720 |
| 3159 | TTATAGCGTTCACAAC | 39201448 | 39201463 | 3643 | ATATGGACGATGCCTG | 38870704 | 38870719 |
| 3160 | TATAGCGTTCACAACT | 39201447 | 39201462 | 3644 | TATGGACGATGCCTGT | 38870703 | 38870718 |
| 3161 | TAGCGTTCACAACTAC | 39201445 | 39201460 | 3645 | ATGGACGATGCCTGTC | 38870702 | 38870717 |
| 3162 | AGCGTTCACAACTACA | 39201444 | 39201459 | 3646 | AGCTCGTCACTTCCAA | 38870480 | 38870495 |
| 3163 | GCGTTCACAACTACAG | 39201443 | 39201458 | 3647 | ACGTCCAGATTGCTGT | 38896324 | 38896339 |
| 3164 | CGTTCACAACTACAGA | 39201442 | 39201457 | 3648 | AGGACCGTATGAACCT | 38865705 | 38865720 |
| 3165 | GACCCTGTCATACGTT | 39199891 | 39199906 | 3649 | GGACCGTATGAACCTG | 38865704 | 38865719 |
| 3166 | ACCCTGTCATACGTTA | 39199890 | 39199905 | 3650 | GACCGTATGAACCTGA | 38865703 | 38865718 |
| 3167 | GTCATACGTTACTTTG | 39199885 | 39199900 | 3651 | ACCGTATGAACCTGAA | 38865702 | 38865717 |
| 3168 | GCATACACGACCTTCC | 39213500 | 39213515 | 3652 | CCGTATGAACCTGAAT | 38865701 | 38865716 |
| 3169 | CATACGTTACTTTGAC | 39199883 | 39199898 | 3653 | CGTATGAACCTGAATC | 38865700 | 38865715 |
| 3170 | ACGCTACTTCCTTAAG | 39199738 | 39199753 | 3654 | AACTGCTAAACGTGAC | 38864854 | 38864869 |
| 3171 | TTCTAAGTCTCGTGCA | 39199287 | 39199302 | 3655 | ACTGCTAAACGTGACT | 38864853 | 38864868 |
| 3172 | TCTAAGTCTCGTGCAA | 39199286 | 39199301 | 3656 | CTGCTAAACGTGACTC | 38864852 | 38864867 |
| 3173 | CTAAGTCTCGTGCAAT | 39199285 | 39199300 | 3657 | TGCTAAACGTGACTCC | 38864851 | 38864866 |
| 3174 | TAAGTCTCGTGCAATT | 39199284 | 39199299 | 3658 | ATTGCTTGTAGTGACC | 39409821 | 39409836 |
| 3175 | AAGTCTCGTGCAATTG | 39199283 | 39199298 | 3659 | TCACAGCCCAATTACC | 39406532 | 39406547 |
| 3176 | AGTCTCGTGCAATTGG | 39199282 | 39199297 | 3660 | GATGTTGCAGGGCTAA | 39405461 | 39405476 |
| 3177 | GTCTCGTGCAATTGGT | 39199281 | 39199296 | 3661 | TGTTGCAGGGCTAATC | 39405459 | 39405474 |
| 3178 | GGAATACGCCACTTTC | 39207771 | 39207786 | 3662 | GTTGCAGGGCTAATCT | 39405458 | 39405473 |
| 3179 | ACTTTCGGCTTAGGTA | 39207761 | 39207776 | 3663 | TTGCAGGGCTAATCTC | 39405457 | 39405472 |
| 3180 | CTTTCGGCTTAGGTAA | 39207760 | 39207775 | 3664 | GCAGGGCTAATCTCAC | 39405455 | 39405470 |
| 3181 | TTTCGGCTTAGGTAAA | 39207759 | 39207774 | 3665 | AAGCTCCCTATGTATG | 39402143 | 39402158 |
| 3182 | TTCGGCTTAGGTAAAG | 39207758 | 39207773 | 3666 | AGCTCCCTATGTATGG | 39402142 | 39402157 |
| 3183 | TCGGCTTAGGTAAAGA | 39207757 | 39207772 | 3667 | GGCCATTACTTATCCA | 39397882 | 39397897 |
| 3184 | TCTCGTGCAATTGGTG | 39199280 | 39199295 | 3668 | GTATACTCAATGGGTA | 39382182 | 39382197 |
| 3185 | CTCGTGCAATTGGTGT | 39199279 | 39199294 | 3669 | ATACTCAATGGGTAGC | 39382180 | 39382195 |
| 3186 | TCGTGCAATTGGTGTC | 39199278 | 39199293 | 3670 | CCATTAGTGGTCTGAA | 39377968 | 39377983 |
| 3187 | CGTGCAATTGGTGTCT | 39199277 | 39199292 | 3671 | TAGCTAGTCTTGAGCA | 39376670 | 39376685 |
| 3188 | ATCGTGTGAGATGAGT | 39198355 | 39198370 | 3672 | GCTAGTCTTGAGCAGT | 39376668 | 39376683 |
| 3189 | TCGTGTGAGATGAGTA | 39198354 | 39198369 | 3673 | GGTTATGCCCATCAAT | 39364968 | 39364983 |
| 3190 | TATCTGAAGGGTTGCG | 39192981 | 39192996 | 3674 | CATATGGACTGGACAC | 39349029 | 39349044 |
| 3191 | ATCTGAAGGGTTGCGG | 39192980 | 39192995 | 3675 | GGGATACTATTGTTAC | 39347792 | 39347807 |
| 3192 | TCTGAAGGGTTGCGGT | 39192979 | 39192994 | 3676 | CAAGCTAAGTCTGGTT | 39342088 | 39342103 |
| 3193 | CTGAAGGGTTGCGGTT | 39192978 | 39192993 | 3677 | TGAGGGTAAGCTGATC | 39332629 | 39332644 |
| 3194 | TGAAGGGTTGCGGTTG | 39192977 | 39192992 | 3678 | AACACTCATACTATCC | 39328319 | 39328334 |
| 3195 | AAGGGTTGCGGTTGAT | 39192975 | 39192990 | 3679 | GTTCTAAGACTAACTC | 39327815 | 39327830 |
| 3196 | AGGGTTGCGGTTGATA | 39192974 | 39192989 | 3680 | GGTGGTGGACATTAAC | 39326669 | 39326684 |
| 3197 | GGGTTGCGGTTGATAA | 39192973 | 39192988 | 3681 | GTCAATATAAGAGACC | 39323395 | 39323410 |
| 3198 | GGTTGCGGTTGATAAA | 39192972 | 39192987 | 3682 | CCCTGGAGCTATATTA | 39322349 | 39322364 |
| 3199 | GTTGCGGTTGATAAAT | 39192971 | 39192986 | 3683 | GAGCTATATTAGGAGT | 39322344 | 39322359 |
| 3200 | TCATTAAGTCCCGTTA | 39192551 | 39192566 | 3684 | AGCTATATTAGGAGTG | 39322343 | 39322358 |
| 3201 | CATTAAGTCCCGTTAG | 39192550 | 39192565 | 3685 | GCTATATTAGGAGTGA | 39322342 | 39322357 |
| 3202 | ATTAAGTCCCGTTAGC | 39192549 | 39192564 | 3686 | GCCTAGATAGCACATG | 39319152 | 39319167 |
| 3203 | TTAAGTCCCGTTAGCA | 39192548 | 39192563 | 3687 | TGTCACAAGGTCTAGG | 39318113 | 39318128 |
| 3204 | TAAGTCCCGTTAGCAA | 39192547 | 39192562 | 3688 | GCAATAGACTTGGGCA | 39313262 | 39313277 |
| 3205 | GTCCCGTTAGCAACTC | 39192544 | 39192559 | 3689 | GCCATAGGTGTGCTTA | 39313193 | 39313208 |
| 3206 | CCGTTAGCAACTCTTG | 39192541 | 39192556 | 3690 | GTAGGTCTGTTCTAAC | 39313129 | 39313144 |
| 3207 | CGTTAGCAACTCTTGA | 39192540 | 39192555 | 3691 | GTCCTCTAGCAATGGT | 39311489 | 39311504 |
| 3208 | CCTTCTGAAATCATCG | 39198367 | 39198382 | 3692 | CTGGTGGATCTTCTAC | 39297078 | 39297093 |
| 3209 | GAAATCATCGTGTGAG | 39198361 | 39198376 | 3693 | GGTGCAACTGTTATGT | 39287977 | 39287992 |
| 3210 | AAATCATCGTGTGAGA | 39198360 | 39198375 | 3694 | CATGCAGTAAGGGATC | 39284896 | 39284911 |
| 3211 | TTGTGGTTAACTTTCG | 39190374 | 39190389 | 3695 | GGGCTATCATTAGTAA | 39348097 | 39348112 |
| 3212 | TGTGGTTAACTTTCGT | 39190373 | 39190388 | 3696 | CTTCAATTGGTGTGCA | 39284505 | 39284520 |
| 3213 | AAGTCCGTGCTTGTCA | 39194771 | 39194786 | 3697 | GGGTAGGTATACTGGT | 39344290 | 39344305 |
| 3214 | GTGGTTAACTTTCGTA | 39190372 | 39190387 | 3698 | GGTAGGTATACTGGTC | 39344289 | 39344304 |
| 3215 | TGGTTAACTTTCGTAG | 39190371 | 39190386 | 3699 | GGTATACTGGTCAATG | 39344285 | 39344300 |
| 3216 | GGTTAACTTTCGTAGA | 39190370 | 39190385 | 3700 | GTATACTGGTCAATGG | 39344284 | 39344299 |
| 3217 | ATATACTCTGGGCTCG | 39184199 | 39184214 | 3701 | GTCACAGGCTAACTTC | 39343823 | 39343838 |
| 3218 | TATACTCTGGGCTCGA | 39184198 | 39184213 | 3702 | TCAATTGGTGTGCAAC | 39284503 | 39284518 |
| 3219 | ATACTCTGGGCTCGAT | 39184197 | 39184212 | 3703 | GTTGAACAGATGGTAC | 39335090 | 39335105 |
| 3220 | TACTCTGGGCTCGATG | 39184196 | 39184211 | 3704 | CAATTGGTGTGCAACA | 39284502 | 39284517 |
| 3221 | ACTCTGGGCTCGATGT | 39184195 | 39184210 | 3705 | TATCCCTCCACATAAG | 39264227 | 39264242 |
| 3222 | CTCTGGGCTCGATGTA | 39184194 | 39184209 | 3706 | GGTTCAATGTTCCATC | 39255324 | 39255339 |
| 3223 | CTCGATGTAAGCCATG | 39184187 | 39184202 | 3707 | CCTATATGGACAATAG | 39253240 | 39253255 |
| 3224 | CCTACAATGACCAGCG | 39178604 | 39178619 | 3708 | GACCAATTCCTGTCTA | 39245140 | 39245155 |
| 3225 | CTACAATGACCAGCGA | 39178603 | 39178618 | 3709 | CCCTTAGATCAGAATC | 39243688 | 39243703 |
| 3226 | ACGTTAATTCCTCATG | 39178099 | 39178114 | 3710 | TAGCTTAACCAGTATG | 39243581 | 39243596 |
| 3227 | CGTTAATTCCTCATGG | 39178098 | 39178113 | 3711 | GCTTAACCAGTATGTC | 39243579 | 39243594 |
| 3228 | AGGTTCAAGTATCACG | 39176620 | 39176635 | 3712 | GGTGTTCCAAATGCAC | 39242191 | 39242206 |
| 3229 | GGTTCAAGTATCACGG | 39176619 | 39176634 | 3713 | GGCATGGCTCTCTTAC | 39239120 | 39239135 |
| 3230 | GTTCAAGTATCACGGT | 39176618 | 39176633 | 3714 | GGGTGAACTCAAATGC | 39318733 | 39318748 |
| 3231 | TTCAAGTATCACGGTT | 39176617 | 39176632 | 3715 | AAGCTCAATCACCTTA | 39239104 | 39239119 |
| 3232 | TCAAGTATCACGGTTG | 39176616 | 39176631 | 3716 | CACTGGTCAGGGTTAT | 39232193 | 39232208 |
| 3233 | CAAGTATCACGGTTGT | 39176615 | 39176630 | 3717 | ACTGGTCAGGGTTATA | 39232192 | 39232207 |
| 3234 | AAGTATCACGGTTGTC | 39176614 | 39176629 | 3718 | TGGTCAGGGTTATAAG | 39232190 | 39232205 |
| 3235 | AAATGTTGGCGACATG | 39176266 | 39176281 | 3719 | GTCAGGGTTATAAGGT | 39232188 | 39232203 |
| 3236 | AATGTTGGCGACATGC | 39176265 | 39176280 | 3720 | TTAGATTGATTGGCAC | 39232022 | 39232037 |
| 3237 | AGAGTCGGTGTGTAAC | 39172548 | 39172563 | 3721 | TCATCAATCTAGCAAC | 39228682 | 39228697 |
| 3238 | GAGTCGGTGTGTAACT | 39172547 | 39172562 | 3722 | ATCAATCTAGCAACAC | 39228680 | 39228695 |
| 3239 | AGTCGGTGTGTAACTG | 39172546 | 39172561 | 3723 | GTATGACACCTGTTGT | 39226327 | 39226342 |
| 3240 | GTCGGTGTGTAACTGC | 39172545 | 39172560 | 3724 | CACTACCTACACACTA | 39211312 | 39211327 |
| 3241 | TCGGTGTGTAACTGCA | 39172544 | 39172559 | 3725 | ACTACCTACACACTAT | 39211311 | 39211326 |
| 3242 | CGGTGTGTAACTGCAA | 39172543 | 39172558 | 3726 | GACATAGGGAGCTATC | 39271670 | 39271685 |
| 3243 | CAAAGACCACAATTCG | 39167514 | 39167529 | 3727 | ACATAGGGAGCTATCT | 39271669 | 39271684 |
| 3244 | AAAGACCACAATTCGT | 39167513 | 39167528 | 3728 | AGGGAGCTATCTATTA | 39271665 | 39271680 |
| 3245 | AGACCACAATTCGTAT | 39167511 | 39167526 | 3729 | GGGAGCTATCTATTAC | 39271664 | 39271679 |
| 3246 | GACCACAATTCGTATG | 39167510 | 39167525 | 3730 | GGAGCTATCTATTACC | 39271663 | 39271678 |
| 3247 | ACCACAATTCGTATGT | 39167509 | 39167524 | 3731 | GGGATAACTCTTATAG | 39205950 | 39205965 |
| 3248 | ACATGTTACGAAGTGA | 39177523 | 39177538 | 3732 | GGATAACTCTTATAGC | 39205949 | 39205964 |
| 3249 | CCGGTTAAGATGCTAC | 39164428 | 39164443 | 3733 | CACCTACCATATTGAG | 39200097 | 39200112 |
| 3250 | CGGTTAAGATGCTACA | 39164427 | 39164442 | 3734 | TGGACTATGGTCAGGT | 39195765 | 39195780 |
| 3251 | TGATGGCGGTGTTGTA | 39158412 | 39158427 | 3735 | GGACTATGGTCAGGTA | 39195764 | 39195779 |
| 3252 | GATGGCGGTGTTGTAC | 39158411 | 39158426 | 3736 | GACTATGGTCAGGTAA | 39195763 | 39195778 |
| 3253 | GTCTGGGAGGAGTTCG | 39146993 | 39147008 | 3737 | TTGGTTAGGGTATTCC | 39188240 | 39188255 |
| 3254 | TCTGGGAGGAGTTCGA | 39146992 | 39147007 | 3738 | TAGGTTCAAGTATCAC | 39176621 | 39176636 |
| 3255 | GCCTGTGCCGTCCAAT | 39144174 | 39144189 | 3739 | GTCAGATCATTGCTAG | 39171656 | 39171671 |
| 3256 | CCAATGTAGGGACCGC | 39132490 | 39132505 | 3740 | AGATCATTGCTAGTAC | 39171653 | 39171668 |
| 3257 | CTCTGAGTGACGTAGC | 39132165 | 39132180 | 3741 | GTACCCAACTTCCTGA | 39171641 | 39171656 |
| 3258 | GCGTGATGGATTGGCT | 39174302 | 39174317 | 3742 | AGGTGGATCATACATC | 39164261 | 39164276 |
| 3259 | CGTGATGGATTGGCTA | 39174301 | 39174316 | 3743 | GATCATACATCCAACT | 39164256 | 39164271 |
| 3260 | GATTGGCTATGTGGCG | 39174294 | 39174309 | 3744 | GGTGTTGTACCCTTGT | 39158405 | 39158420 |
| 3261 | ATTGGCTATGTGGCGT | 39174293 | 39174308 | 3745 | GTGTTGTACCCTTGTG | 39158404 | 39158419 |
| 3262 | TTGGCTATGTGGCGTA | 39174292 | 39174307 | 3746 | GACCAGCACCCTTAAG | 39140572 | 39140587 |
| 3263 | GCTATGTGGCGTAAGG | 39174289 | 39174304 | 3747 | CAGTAGATTGTTAGAC | 39134307 | 39134322 |
| 3264 | CTATGTGGCGTAAGGG | 39174288 | 39174303 | 3748 | AGTAGATTGTTAGACC | 39134306 | 39134321 |
| 3265 | TCTGAGTGACGTAGCA | 39132164 | 39132179 | 3749 | GCAACTCAATACTCTC | 39214133 | 39214148 |
| 3266 | GTGACGTAGCACCTAT | 39132159 | 39132174 | 3750 | CTGGTCATGGGCATAC | 39213510 | 39213525 |
| 3267 | GACGTAGCACCTATCT | 39132157 | 39132172 | 375 | GTAGATTGTTAGACCC | 39134305 | 39134320 |
| 3268 | ACGTAGCACCTATCTG | 39132156 | 39132171 | 3752 | GCTCTCATAGGGTATA | 39122090 | 39122105 |
| 3269 | CGTAGCACCTATCTGA | 39132155 | 39132170 | 3753 | GTGATCCAGCTTGCAA | 39208745 | 39208760 |
| 3270 | CCATTTCGTGGTTGGA | 39124503 | 39124518 | 3754 | GATCCAGCTTGCAATA | 39208743 | 39208758 |
| 3271 | AATGGCTATTCTCCGT | 39171491 | 39171506 | 3755 | CTCTCATAGGGTATAG | 39122089 | 39122104 |
| 3272 | GGCTATTCTCCGTCAA | 39171488 | 39171503 | 3756 | GTAGGATAACTCAGAC | 39115396 | 39115411 |
| 3273 | CATTTCGTGGTTGGAT | 39124502 | 39124517 | 3757 | GACTATACAAGTAGGT | 39115163 | 39115178 |
| 3274 | TTTCGTGGTTGGATTG | 39124500 | 39124515 | 3758 | CTATACAAGTAGGTGC | 39115161 | 39115176 |
| 3275 | TTCGTGGTTGGATTGG | 39124499 | 39124514 | 3759 | ATACAAGTAGGTGCTC | 39115159 | 39115174 |
| 3276 | TAGGCTGTTTCTTACG | 39122434 | 39122449 | 3760 | GCCTCTCCCAAGTTAG | 39114231 | 39114246 |
| 3277 | TACCCGCTCTCATAGG | 39122095 | 39122110 | 3761 | GTCACTCCAACTTGAT | 39194759 | 39194774 |
| 3278 | CCGCTCTCATAGGGTA | 39122092 | 39122107 | 3762 | GTGGTGTGCTTAGTCC | 39091762 | 39091777 |
| 3279 | CGCTCTCATAGGGTAT | 39122091 | 39122106 | 3763 | CCTTGAGGTTGTCCAC | 39091380 | 39091395 |
| 3280 | TATGGAGCGTAGCAGC | 39121181 | 39121196 | 3764 | GTCCTGTGCAATTAGT | 39175217 | 39175232 |
| 3281 | AGCAGCCAACAGTTCG | 39121171 | 39121186 | 3765 | TCCTGTGCAATTAGTC | 39175216 | 39175231 |
| 3282 | GACATAGTGTCCCGCC | 39150933 | 39150948 | 3766 | CCTGTGCAATTAGTCC | 39175215 | 39175230 |
| 3283 | ACATAGTGTCCCGCCC | 39150932 | 39150947 | 3767 | CTGTGCAATTAGTCCC | 39175214 | 39175229 |
| 3284 | CATAGTGTCCCGCCCA | 39150931 | 39150946 | 3768 | TAACACTTGATCTGAC | 39174770 | 39174785 |
| 3285 | ATAGTGTCCCGCCCAT | 39150930 | 39150945 | 3769 | ATCTGACCACCAACTA | 39174761 | 39174776 |
| 3286 | AACGTTGTCTCCTGTA | 39150868 | 39150883 | 3770 | CTGACCACCAACTAAC | 39174759 | 39174774 |
| 3287 | ACGTTGTCTCCTGTAG | 39150867 | 39150882 | 3771 | TGACCACCAACTAACA | 39174758 | 39174773 |
| 3288 | TAAATACGTTGTGGCT | 39150800 | 39150815 | 3772 | CACCAACTAACAATAG | 39174754 | 39174769 |
| 3289 | AATACGTTGTGGCTAT | 39150798 | 39150813 | 3773 | CCAACTAACAATAGGG | 39174752 | 39174767 |
| 3290 | ATACGTTGTGGCTATG | 39150797 | 39150812 | 3774 | GTAATAGTCTAACTGC | 39174400 | 39174415 |
| 3291 | TACGTTGTGGCTATGG | 39150796 | 39150811 | 3775 | AATAGTCTAACTGCAC | 39174398 | 39174413 |
| 3292 | ACGTTGTGGCTATGGG | 39150795 | 39150810 | 3776 | ATAGTCTAACTGCACA | 39174397 | 39174412 |
| 3293 | TGCTGCATCCCGTAGA | 39118125 | 39118140 | 3777 | GGCCCTAGTGTCATAC | 39174131 | 39174146 |
| 3294 | TCAACCTTGTCTCGGA | 39116381 | 39116396 | 3778 | GCCCTAGTGTCATACC | 39174130 | 39174145 |
| 3295 | AGGAAATACGCTCAAT | 39145505 | 39145520 | 3779 | CCCTAGTGTCATACCA | 39174129 | 39174144 |
| 3296 | GGAAATACGCTCAATT | 39145504 | 39145519 | 3780 | CCTAGTGTCATACCAT | 39174128 | 39174143 |
| 3297 | ACGCTCAATTATTGTG | 39145498 | 39145513 | 3781 | GAGGTGACTGACTAGC | 39173804 | 39173819 |
| 3298 | CGCTCAATTATTGTGA | 39145497 | 39145512 | 3782 | CAGGCTGCTACATTAC | 39088324 | 39088339 |
| 3299 | CAACCTTGTCTCGGAT | 39116380 | 39116395 | 3783 | GCTGCTACATTACCTA | 39088321 | 39088336 |
| 3300 | CGGTCACAATTCTATT | 39143113 | 39143128 | 3784 | GCTACATTACCTAAAC | 39088318 | 39088333 |
| 3301 | GAATTCAAACGGTAGT | 39136340 | 39136355 | 3785 | CTAACTGAGGTGTGAC | 39077718 | 39077733 |
| 3302 | ACGGTAGTTTGATTGA | 39136332 | 39136347 | 3786 | GGTTACCACTCAAGAT | 39074683 | 39074698 |
| 3303 | AACCTTGTCTCGGATC | 39116379 | 39116394 | 3787 | TAACTCACCCAATAGC | 39074592 | 39074607 |
| 3304 | GGATAACTCAGACGGT | 39115393 | 39115408 | 3788 | AACTCACCCAATAGCC | 39074591 | 39074606 |
| 3305 | GATAACTCAGACGGTT | 39115392 | 39115407 | 3789 | GACCCTAATTCAGCAC | 39073669 | 39073684 |
| 3306 | ATAACTCAGACGGTTG | 39115391 | 39115406 | 3790 | GTGTGGTGCAGCTAAT | 39073377 | 39073392 |
| 3307 | TAACTCAGACGGTTGA | 39115390 | 39115405 | 3791 | TGTGGTGCAGCTAATC | 39073376 | 39073391 |
| 3308 | AACTCAGACGGTTGAA | 39115389 | 39115404 | 3792 | GTGGTGCAGCTAATCA | 39073375 | 39073390 |
| 3309 | ACTCAGACGGTTGAAC | 39115388 | 39115403 | 3793 | GGCTATTGCTCATGTC | 39068626 | 39068641 |
| 3310 | AGAGAACCTGACTGCG | 39114766 | 39114781 | 3794 | CAATCCAGTTATGTAC | 39067265 | 39067280 |
| 3311 | TACGTACACCAGCAGT | 39114613 | 39114628 | 3795 | GGTATTGTCCTACTTG | 39064547 | 39064562 |
| 3312 | CTGAGTCACGCTGAAA | 39113942 | 39113957 | 3796 | GTTGTTATGACAGGAC | 39055106 | 39055121 |
| 3313 | TGAGTCACGCTGAAAG | 39113941 | 39113956 | 3797 | GTTATGACAGGACTGG | 39055103 | 39055118 |
| 3314 | AGCGTGAGGTGCTTCC | 39110290 | 39110305 | 3798 | TATGACAGGACTGGTA | 39055101 | 39055116 |
| 3315 | GCGTGAGGTGCTTCCT | 39110289 | 39110304 | 3799 | CCCTTTACAACAGACC | 39053137 | 39053152 |
| 3316 | CGTGTTGCATAGGTAA | 39109650 | 39109665 | 3800 | GATGGGTGATTCCTAG | 39037972 | 39037987 |
| 3317 | CAGAAATCAGTCACGC | 39107997 | 39108012 | 3801 | TACCAAACCTACTATG | 39022027 | 39022042 |
| 3318 | AGAAATCAGTCACGCC | 39107996 | 39108011 | 3802 | GAGGGTATGTTAATCC | 39016425 | 39016440 |
| 3319 | GAAATCAGTCACGCCT | 39107995 | 39108010 | 3803 | TAAGGTGCATAAGTTG | 39014400 | 39014415 |
| 3320 | AAATCAGTCACGCCTA | 39107994 | 39108009 | 3804 | ACCAACTATCACCAAC | 38997508 | 38997523 |
| 3321 | AATCAGTCACGCCTAG | 39107993 | 39108008 | 3805 | CTATCACCAACCAATC | 38997503 | 38997518 |
| 3322 | ATCAGTCACGCCTAGA | 39107992 | 39108007 | 3806 | ACCAATCTGTCCCAAT | 38997494 | 38997509 |
| 3323 | TCAGTCACGCCTAGAT | 39107991 | 39108006 | 3807 | ACTGACTATATGGAGC | 38994744 | 38994759 |
| 3324 | CAGTCACGCCTAGATA | 39107990 | 39108005 | 3808 | GAGCCAGATGATACAC | 38994712 | 38994727 |
| 3325 | AGTCACGCCTAGATAC | 39107989 | 39108004 | 3809 | GCCAGATGATACACAC | 38994710 | 38994725 |
| 3326 | GTCACGCCTAGATACA | 39107988 | 39108003 | 3810 | GTGACAGGATTGGTAC | 38994327 | 38994342 |
| 3327 | TCACGCCTAGATACAT | 39107987 | 39108002 | 3811 | GGGCACTTTAGGGTAT | 38990437 | 38990452 |
| 3328 | GTTAATCTTTAGCCGG | 39107228 | 39107243 | 3812 | GGCACTTTAGGGTATC | 38990436 | 38990451 |
| 3329 | TTATTGGGTCATCGTA | 39106613 | 39106628 | 3813 | ATTAGATCATACCAGC | 39071531 | 39071546 |
| 3330 | TATTGGGTCATCGTAC | 39106612 | 39106627 | 3814 | TTAGATCATACCAGCC | 39071530 | 39071545 |
| 3331 | ATTGGGTCATCGTACT | 39106611 | 39106626 | 3815 | GCACTTTAGGGTATCT | 38990435 | 38990450 |
| 3332 | TTGGGTCATCGTACTC | 39106610 | 39106625 | 3816 | CACCTAATGGACTGCA | 38983021 | 38983036 |
| 3333 | GGGTCATCGTACTCCA | 39106608 | 39106623 | 3817 | ACAAACCCAATAGACC | 38982762 | 38982777 |
| 3334 | GGTCATCGTACTCCAT | 39106607 | 39106622 | | CAAACCCAATAGACCT | 38982761 | 38982776 |
| 3335 | CCGGACAATTATAGAA | 39099316 | 39099331 | 3819 | AACCCAATAGACCTTA | 38982759 | 38982774 |
| 3336 | CGGACAATTATAGAAC | 39099315 | 39099330 | 3820 | ACCCAATAGACCTTAC | 38982758 | 38982773 |
| 3337 | TAATGAACTATCCGAG | 39099095 | 39099110 | 3821 | CCCAATAGACCTTACA | 38982757 | 38982772 |
| 3338 | AATGAACTATCCGAGC | 39099094 | 39099109 | 3822 | CCAATAGACCTTACAC | 38982756 | 38982771 |
| 3339 | ATGAACTATCCGAGCA | 39099093 | 39099108 | 3823 | ATAGACCTTACACAAG | 38982753 | 38982768 |
| 3340 | TGAACTATCCGAGCAC | 39099092 | 39099107 | 3824 | CATAGAGTTGAAGCAC | 39020361 | 39020376 |
| 3341 | GAACTATCCGAGCACC | 39099091 | 39099106 | 3825 | CAATCAGCAAGGGTAT | 38981735 | 38981750 |
| 3342 | TTTGCGAGTCAGTAGG | 39095831 | 39095846 | 3826 | GGTTAATAGAAGCCTA | 38977637 | 38977652 |
| 3343 | TTGCGAGTCAGTAGGA | 39095830 | 39095845 | 3827 | GTATACCTAAAGCCCA | 38951242 | 38951257 |
| 3344 | TGCGAGTCAGTAGGAT | 39095829 | 39095844 | 3828 | TATACCTAAAGCCCAC | 38951241 | 38951256 |
| 3345 | GCGAGTCAGTAGGATT | 39095828 | 39095843 | 3829 | GTTATATGGTCTGGGC | 38949645 | 38949660 |
| 3346 | CGAGTCAGTAGGATTT | 39095827 | 39095842 | 3830 | TGTATTGAACCCTAGC | 38997215 | 38997230 |
| 3347 | GTCCACACGATACTTA | 39091370 | 39091385 | 3831 | TTTGGTACTCTACAAC | 38946797 | 38946812 |
| 3348 | TCCACACGATACTTAT | 39091369 | 39091384 | 3832 | GACTACCACAATTCAT | 38945462 | 38945477 |
| 3349 | CCACACGATACTTATT | 39091368 | 39091383 | 3833 | GGTCTGTTACCCATTG | 38943391 | 38943406 |
| 3350 | GAATACCCACGTTTGA | 39087154 | 39087169 | 3834 | CCAGCTCATTAGGCAC | 38941737 | 38941752 |
| 3351 | AATACCCACGTTTGAT | 39087153 | 39087168 | 3835 | AGCTCATTAGGCACAC | 38941735 | 38941750 |
| 3352 | ATACCCACGTTTGATC | 39087152 | 39087167 | 3836 | GCTCATTAGGCACACT | 38941734 | 38941749 |
| 3353 | ACCCACGTTTGATCTG | 39087150 | 39087165 | 3837 | CAGGGAGCCTTAACTA | 38941586 | 38941601 |
| 3354 | CACTTTAGCATGAACG | 39086770 | 39086785 | 3838 | AGGGAGCCTTAACTAC | 38941585 | 38941600 |
| 3355 | ACTTTAGCATGAACGT | 39086769 | 39086784 | 3839 | CTCAAGATTGACTAAC | 38936002 | 38936017 |
| 3356 | CTTTAGCATGAACGTG | 39086768 | 39086783 | 3840 | GGACCTCCATACACAT | 38932740 | 38932755 |
| 3357 | TTTAGCATGAACGTGA | 39086767 | 39086782 | 3841 | CTGATCTGAACAACCC | 38932509 | 38932524 |
| 3358 | TTAGCATGAACGTGAG | 39086766 | 39086781 | 3842 | GTCAAACTAAGGTTGT | 38930693 | 38930708 |
| 3359 | TTCAAAGTGTGCGTTC | 39085532 | 39085547 | 3843 | ACTAAGGTTGTAAGGC | 38930688 | 38930703 |
| 3360 | CGTTCTGTGTATAAAC | 39085521 | 39085536 | 3844 | CTAAGGTTGTAAGGCA | 38930687 | 38930702 |
| 3361 | CTCCTGTTATACGTGG | 39080554 | 39080569 | 3845 | ACTGTATAGTGTAACC | 38924783 | 38924798 |
| 3362 | TCCTGTTATACGTGGT | 39080553 | 39080568 | 3846 | ATATCTAGTCTGGGTC | 38924232 | 38924247 |
| 3363 | CTGTTATACGTGGTCC | 39080551 | 39080566 | 3847 | TATCTAGTCTGGGTCC | 38924231 | 38924246 |
| 3364 | TGTTATACGTGGTCCT | 39080550 | 39080565 | 3848 | TTACTAGGGTAGATGC | 38916341 | 38916356 |
| 3365 | GTTATACGTGGTCCTC | 39080549 | 39080564 | 3849 | GTATTGCTACCTTACA | 38915636 | 38915651 |
| 3366 | GGACCCGCAATATAGA | 39080412 | 39080427 | 3850 | GCTACCTTACAAAGAC | 38915631 | 38915646 |
| 3367 | GACCCGCAATATAGAG | 39080411 | 39080426 | 3851 | TCAGTCTAAGTGTAGC | 38914692 | 38914707 |
| 3368 | CCGCAATATAGAGGAC | 39080408 | 39080423 | 3852 | CAGTCTAAGTGTAGCA | 38914691 | 38914706 |
| 3369 | CGCAATATAGAGGACC | 39080407 | 39080422 | 3853 | AGTCTAAGTGTAGCAC | 38914690 | 38914705 |
| 3370 | GAGCGTTTGTGTCTAC | 39078743 | 39078758 | 3854 | GTCTAAGTGTAGCACC | 38914689 | 38914704 |
| 3371 | AGCGTTTGTGTCTACA | 39078742 | 39078757 | 3855 | GTAGCACCTTATGTAT | 38914681 | 38914696 |
| 3372 | GCCAATTACATCAACG | 39077486 | 39077501 | 3856 | TCTATGGGCTACAACA | 38908866 | 38908881 |
| 3373 | CCAATTACATCAACGG | 39077485 | 39077500 | 3857 | CTATGGGCTACAACAT | 38908865 | 38908880 |
| 3374 | TTTGTAGCGTGTAACC | 39075798 | 39075813 | 3858 | CAACTAAACCCACATC | 38903241 | 38903256 |
| 3375 | TTGTAGCGTGTAACCC | 39075797 | 39075812 | 3859 | AATTGTAGTTAGCCCA | 38891170 | 38891185 |
| 3376 | AACCACAGACGTTGAA | 39075440 | 39075455 | 3860 | GTAGTTAGCCCATATC | 38891166 | 38891181 |
| 3377 | ATTCACCAAACCTTCG | 39074699 | 39074714 | 3861 | TAGTTAGCCCATATCC | 38891165 | 38891180 |
| 3378 | ACAGATAAACTTTCCG | 39074491 | 39074506 | 3862 | AGTTAGCCCATATCCA | 38891164 | 38891179 |
| 3379 | CAGATAAACTTTCCGA | 39074490 | 39074505 | 3863 | TCAGGGTAGATTACTC | 38889602 | 38889617 |
| 3380 | GAAGTGTTGAACGTAC | 39073201 | 39073216 | 3864 | TAAAGGACTAACACCC | 38887618 | 38887633 |
| 3381 | AAGTGTTGAACGTACA | 39073200 | 39073215 | 3865 | CAGATGCTAGGGCTAA | 38887240 | 38887255 |
| 3382 | CGTACAACAGATTCCT | 39073190 | 39073205 | 3866 | ACAAGCCAAGTCCCAC | 38879984 | 38879999 |
| 3383 | TTAGAAATACGGTGAC | 39070733 | 39070748 | 3867 | CTGGTCTGCTACAATG | 38879838 | 38879853 |
| 3384 | TGGTTTAGAGAGCGGT | 39070699 | 39070714 | 3868 | GGTCTGCTACAATGAT | 38879836 | 38879851 |
| 3385 | GGTTTAGAGAGCGGTT | 39070698 | 39070713 | 3869 | GTCTGCTACAATGATC | 38879835 | 38879850 |
| 3386 | GTTTAGAGAGCGGTTA | 39070697 | 39070712 | 3870 | TCTGCTACAATGATCC | 38879834 | 38879849 |
| 3387 | GAGCGGTTAGATTGAT | 39070690 | 39070705 | 3871 | AATTGCAAGTAGACCC | 38877804 | 38877819 |
| 3388 | AGCGGTTAGATTGATG | 39070689 | 39070704 | 3872 | ATTGCAAGTAGACCCA | 38877803 | 38877818 |
| 3389 | GCTGCCGTAAGTTATC | 39070055 | 39070070 | 3873 | TAGGAACTGTTTAGCC | 38877760 | 38877775 |
| 3390 | CTGCCGTAAGTTATCA | 39070054 | 39070069 | 3874 | TGGGTCTAAGATAGGC | 38874245 | 38874260 |
| 3391 | TGCCGTAAGTTATCAC | 39070053 | 39070068 | 3875 | CTCAAACTAGGTAGCA | 38867387 | 38867402 |
| 3392 | GCCGTAAGTTATCACA | 39070052 | 39070067 | 3876 | TCAAACTAGGTAGCAC | 38867386 | 38867401 |
| 3393 | CGTAAGTTATCACAGA | 39070050 | 39070065 | 3877 | CAAACTAGGTAGCACT | 38867385 | 38867400 |
| 3394 | TAACCAGGGACGCAGT | 39067992 | 39068007 | 3878 | AAACTAGGTAGCACTC | 38867384 | 38867399 |
| 3395 | ACCAGGGACGCAGTGT | 39067990 | 39068005 | 3879 | GTAGCACTCCCTCATA | 38867377 | 38867392 |
| 3396 | CAGAGCGTAACAAACC | 39063506 | 39063521 | 3880 | TAGCACTCCCTCATAG | 38867376 | 38867391 |
| 3397 | GCGTAACAAACCAGAC | 39063502 | 39063517 | 3881 | AGCACTCCCTCATAGC | 38867375 | 38867390 |
| 3398 | CGTAACAAACCAGACA | 39063501 | 39063516 | 3882 | CACTTCATGGGTCTAG | 38866541 | 38866556 |
| 3399 | AGATGGTCTCACAACG | 39063410 | 39063425 | 3883 | GTATGAACCTGAATCC | 38865699 | 38865714 |
| 3400 | GATGGTCTCACAACGG | 39063409 | 39063424 | 3884 | GCCGTAGAGAGCAACC | 39427180 | 39427195 |
| 3401 | ATGGTCTCACAACGGC | 39063408 | 39063423 | 3885 | CCGTAGAGAGCAACCA | 39427179 | 39427194 |
| 3402 | TCGTTACAACTCACTG | 39058370 | 39058385 | 3886 | TCGGTCAGATGGAAAC | 39418247 | 39418262 |
| 3403 | CATTACTGTATTGCCG | 39050793 | 39050808 | 3887 | GCCACAAGAGACCACG | 39406674 | 39406689 |
| 3404 | ACTGTATTGCCGTCTA | 39050789 | 39050804 | 3888 | CCACAAGAGACCACGA | 39406673 | 39406688 |
| 3405 | CTGTTGGTTGATTCCG | 39042125 | 39042140 | 3889 | ACGTTCCATGTAGTTC | 39402672 | 39402687 |
| 3406 | TGTTGGTTGATTCCGC | 39042124 | 39042139 | 3890 | CATGTAGTTCCGTGGA | 39402666 | 39402681 |
| 3407 | TTGGTTGATTCCGCAC | 39042122 | 39042137 | 3891 | ATGTAGTTCCGTGGAT | 39402665 | 39402680 |
| 3408 | GGTTGATTCCGCACCT | 39042120 | 39042135 | 3892 | TGTAGTTCCGTGGATG | 39402664 | 39402679 |
| 3409 | TGATTCCGCACCTTGA | 39042117 | 39042132 | 3893 | GTAGTTCCGTGGATGC | 39402663 | 39402678 |
| 3410 | GATTCCGCACCTTGAC | 39042116 | 39042131 | 3894 | TAGTTCCGTGGATGCC | 39402662 | 39402677 |
| 3411 | CCGCACCTTGACTTGA | 39042112 | 39042127 | 3895 | AGTTCCGTGGATGCCC | 39402661 | 39402676 |
| 3412 | CGCACCTTGACTTGAA | 39042111 | 39042126 | 3896 | GGTTCCACCAGACGAT | 39402448 | 39402463 |
| 3413 | TCCTAGAGAAGACGAT | 39037962 | 39037977 | 3897 | ACCAGACGATGCCAGA | 39402442 | 39402457 |
| 3414 | AGACAGCATTGACTCG | 39068595 | 39068610 | 3898 | CCAGACGATGCCAGAA | 39402441 | 39402456 |
| 3415 | GACAGCATTGACTCGA | 39068594 | 39068609 | 3899 | CAGACGATGCCAGAAT | 39402440 | 39402455 |
| 3416 | ACAGCATTGACTCGAT | 39068593 | 39068608 | 3900 | GACGATGCCAGAATAT | 39402438 | 39402453 |
| 3417 | CATTGACTCGATGGTA | 39068589 | 39068604 | 3901 | ACGATGCCAGAATATC | 39402437 | 39402452 |
| 3418 | ATTGACTCGATGGTAC | 39068588 | 39068603 | 3902 | CGATGCCAGAATATCT | 39402436 | 39402451 |
| 3419 | TTGACTCGATGGTACT | 39068587 | 39068602 | 3903 | GCGTCTCTGCTGATTG | 39395200 | 39395215 |

Note: ASOs listed in Table 73 are designed to target IPF risk variants and IPF transplant free survival variants associated with PUFIN2, except for SEQ ID NOs: 2939-2940, 2961-2963, 2971-2974, 3008-3013, 3019-3020, 3029-3032, 3043-3047, 3064-3066, 3068-3078, 3108-3114, 3127-3168, 3178-3183, 3208-3213, 3248, 3258-3264, 3271-3272, 3282-3292, 3295-3298, 3300-3302, 3414-3425, 3428-3431, 3478-3479, 3540-3546, 3587-3592, 3647, 3695, 3697-3701, 3703, 3714, 3726-3730, 3749-3750, 3753-3754, 3761, 3764-3781, 3813-3814, 3824, and 3830, which target only IPF transplant free survival variants, and except for SEQ ID NO: 2990, which targets only IPF risk variants.

TABLE 74
Sequences of ASOs designed to target IPF risk variants and IPF transplant free survival
variants associated with PUFIN4.

| SEQ | | | | SEQ | | | |
|---|---|---|---|---|---|---|---|
| ID | | chr_ | chr_ | ID | | chr_ | chr_ |
| NO: | Sequence | start | end | NO: | Sequence | start | end |
| 3904 | CGTGAGTGTCCAGAGA | 169188188 | 169188203 | 3924 | AAGACACCAACCTACG | 169183373 | 169183388 |
| 3905 | GTCCCGTAGCACCTAA | 169187241 | 169187256 | 3925 | AGACACCAACCTACGG | 169183372 | 169183387 |
| 3906 | TCCCGTAGCACCTAAA | 169187240 | 169187255 | 3926 | GACACCAACCTACGGA | 169183371 | 169183386 |
| 3907 | CCCGTAGCACCTAAAT | 169187239 | 169187254 | 3927 | ACACCAACCTACGGAC | 169183370 | 169183385 |
| 3908 | CCGTAGCACCTAAATA | 169187238 | 169187253 | 3928 | CACCAACCTACGGACT | 169183369 | 169183384 |
| 3909 | CGTAGCACCTAAATAA | 169187237 | 169187252 | 3929 | ACCAACCTACGGACTC | 169183368 | 169183383 |
| 3910 | GATGCCCACCTCGTCA | 169187040 | 169187055 | 3930 | ATGCGGGACAATTTGG | 169182469 | 169182484 |
| 3911 | AGCTTCTGTCCGTGGA | 169187003 | 169187018 | 3931 | TGCGGGACAATTTGGA | 169182468 | 169182483 |
| 3912 | CTTCTGTCCGTGGAGG | 169187001 | 169187016 | 3932 | CGGGACAATTTGGAAC | 169182466 | 169182481 |
| 3913 | GTCCGTGGAGGTGGTT | 169186996 | 169187011 | 3933 | CCAATGGACAGCTCGT | 169180403 | 169180418 |
| 3914 | CCGTGGAGGTGGTTGA | 169186994 | 169187009 | 3934 | CAATGGACAGCTCGTG | 169180402 | 169180417 |
| 3915 | TGGAATGGATTCAGCG | 169185896 | 169185911 | 3935 | CGTGAGACTTTGCATG | 169179706 | 169179721 |
| 3916 | GGAATGGATTCAGCGT | 169185895 | 169185910 | 3936 | ATCGTGTATTACCATC | 169178240 | 169178255 |
| 3917 | GAATGGATTCAGCGTG | 169185894 | 169185909 | 3937 | TCGTGTATTACCATCA | 169178239 | 169178254 |
| 3918 | AATGGATTCAGCGTGC | 169185893 | 169185908 | 3938 | CGTGTATTACCATCAT | 169178238 | 169178253 |
| 3919 | ATGGATTCAGCGTGCT | 169185892 | 169185907 | 3939 | TCCGAAGTATCTCACT | 169177697 | 169177712 |
| 3920 | GTGATGTGGACTAAAC | 169184294 | 169184309 | 3940 | CCGAAGTATCTCACTC | 169177696 | 169177711 |
| 3921 | AGTGTACCATCAGGAG | 169184080 | 169184095 | 3941 | CATCCTCAGCAATTCG | 169173146 | 169173161 |
| 3922 | ACAAGACACCAACCTA | 169183375 | 169183390 | 3942 | GCCCTTCTAACACAGT | 169172260 | 169172275 |
| 3923 | CAAGACACCAACCTAC | 169183374 | 169183389 | 3943 | CCGGAGTCTGCCTTCA | 169172144 | 169172159 |

Note: ASOs listed in Table 74 are designed to target IPF risk variants and IPF transplant free survival variants associated with PUFIN4, except for SEQ ID NOs: 3905-3909, which target only IPF transplant free survival variants.

TABLE 75
Sequences of ASOs designed to target IPF risk variants and IPF transplant free survival
variants associated with PUFIN6.

| SEQ | | | | SEQ | | | |
|---|---|---|---|---|---|---|---|
| ID | | chr_ | chr_ | ID | | chr_ | chr_ |
| NO: | Sequence | start | end | NO: | Sequence | start | end |
| 3944 | CCAGCAACACTCTAGC | 50200989 | 50201004 | 3946 | GCCCTGTAGTTGATCC | 50208403 | 50208418 |
| 3945 | GGCCCTGTAGTTGATC | 50208402 | 50208417 | 3947 | GTAGTAGTGAGACCAT | 50208629 | 50208644 |

Note: ASOs listed in Table 75 are designed to target IPF risk variants and IPF transplant free survival variants associated with PUFIN6, except for SEQ ID NO: 3944, which targets only IPF transplant free survival variants.

TABLE 76
Sequences of ASOs designed to target IPF risk variants and IPF transplant free survival
variants associated with PUFIN8.

| SEQ | | | | SEQ | | | |
|---|---|---|---|---|---|---|---|
| ID | | chr_ | chr_ | ID | | chr_ | chr_ |
| NO: | Sequence | start | end | NO: | Sequence | start | end |
| 3948 | ATAGGCAGGTGGCAGAACTT | 74552294 | 74552313 | 3967 | AGTGGGTACAAGGATTCTAT | 74554680 | 74554699 |
| 3949 | TAGGCAGGTGGCAGAACTTC | 74552295 | 74552314 | 3968 | GTGGGTACAAGGATTCTATT | 74554681 | 74554700 |
| 3950 | GGCTGCATTCATCTCAAGGG | 74553152 | 74553171 | 3969 | TGGGTACAAGGATTCTATTT | 74554682 | 74554701 |
| 3951 | GCTGCATTCATCTCAAGGGC | 74553153 | 74553172 | 3970 | GGGTACAAGGATTCTATTTG | 74554683 | 74554702 |
| 3952 | CTGCATTCATCTCAAGGGCA | 74553154 | 74553173 | 3971 | GGTACAAGGATTCTATTTGG | 74554684 | 74554703 |
| 3953 | TGCATTCATCTCAAGGGCAT | 74553155 | 74553174 | 3972 | GTACAAGGATTCTATTTGGA | 74554685 | 74554704 |
| 3954 | GCATTCATCTCAAGGGCATT | 74553156 | 74553175 | 3973 | TACAAGGATTCTATTTGGAG | 74554686 | 74554705 |
| 3955 | CATTCATCTCAAGGGCATTT | 74553157 | 74553176 | 3974 | ACAAGGATTCTATTTGGAGT | 74554687 | 74554706 |
| 3956 | ATTCATCTCAAGGGCATTTG | 74553158 | 74553177 | 3975 | CAAGGATTCTATTTGGAGTG | 74554688 | 74554707 |
| 3957 | TTCATCTCAAGGGCATTTGC | 74553159 | 74553178 | 3976 | AAGGATTCTATTTGGAGTGA | 74554689 | 74554708 |
| 3958 | TAGTGAGTTGTTATCATGCC | 74554439 | 74554458 | 3977 | AGGATTCTATTTGGAGTGAT | 74554690 | 74554709 |
| 3959 | TGAGTTGTTATCATGCCACC | 74554442 | 74554461 | 3978 | GGATTCTATTTGGAGTGATA | 74554691 | 74554710 |
| 3960 | GCTGCTTAGTGGGTACAAGG | 74554673 | 74554692 | 3979 | CTCAGGCACAGATGTAAACA | 74554860 | 74554879 |
| 3961 | CTGCTTAGTGGGTACAAGGA | 74554674 | 74554693 | 3980 | TCAGGCACAGATGTAAACAG | 74554861 | 74554880 |
| 3962 | TGCTTAGTGGGTACAAGGAT | 74554675 | 74554694 | 3981 | CAGTTCTGTTAGCCCTTGGT | 74558043 | 74558062 |
| 3963 | GCTTAGTGGGTACAAGGATT | 74554676 | 74554695 | 3982 | AGTTCTGTTAGCCCTTGGTG | 74558044 | 74558063 |
| 3964 | CTTAGTGGGTACAAGGATTC | 74554677 | 74554696 | 3983 | GTTCTGTTAGCCCTTGGTGA | 74558045 | 74558064 |
| 3965 | TTAGTGGGTACAAGGATTCT | 74554678 | 74554697 | 3984 | AGAGAGGTGGCTGAACTGCT | 74558458 | 74558477 |
| 3966 | TAGTGGGTACAAGGATTCTA | 74554679 | 74554698 | 3985 | CGAGAACAGTGAGACCATGC | 74559010 | 74559029 |

Note: ASOs listed in Table 76 are designed to target IPF risk variants and IPF transplant free survival variants associated with PUFIN8, except for SEQ ID NOs: 3950-3957 and 3979-3980, which target only IPF transplant free survival variants.

TABLE 77
Sequences of ASOs designed to target IPF risk variants and IPF transplant free
survival variants associated with PUFIN10.

| SEQ | | | | SEQ | | | |
|---|---|---|---|---|---|---|---|
| ID | | chr_ | chr_ | ID | | chr_ | chr_ |
| NO: | Sequence | start | end | NO: | Sequence | start | end |
| 3986 | GCGCCTTCAACTGTGA | 1219989 | 1220004 | 3987 | CGCCTTCAACTGTGAA | 1219990 | 1220005 |

Note: ASOs listed in Table 77 are designed to target IPF risk variants and IPF transplant free survival variants associated with PUFIN10.

1. A synthetic antisense oligonucleotide of a long noncoding RNA (lncRNA), wherein the lncRNA is transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +); chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +), relative to human genome GRCh38.p14 (GCF_000001405.40), wherein the lncRNA is a pulmonary-expressed lncRNA.

2. (canceled)

3. The synthetic antisense oligonucleotide of claim 1, wherein the region comprises one or more genetic variants selected from one or more single nucleotide polymorphisms (SNPs), indel variations, copy number variations (CNV), or a combination thereof.

4. The synthetic antisense oligonucleotide of claim 3, wherein the one or more SNPs comprises rs150797, rs220262, rs220249, rs2292305, rs3763267, rs2586502, rs61980882, rs37505950, rs150794, or a combination thereof.

5. (canceled)

6. The synthetic antisense oligonucleotide of claim 1, wherein the synthetic antisense oligonucleotide comprises a nucleic acid sequence that is complementary to at least 13 contiguous nucleotides of the lncRNA.

7. The synthetic antisense oligonucleotide of claim 6, wherein the synthetic or artificial antisense oligonucleotide is about 13-35, or about 16-20 nucleotides in length.

8. (canceled)

9. The synthetic antisense oligonucleotide of claim 1, wherein the synthetic antisense oligonucleotide comprises i) a GapmeR comprising a central region of consecutive DNA nucleotides flanked by a 5′-wing region and 3′-wing region, wherein at least one of 5′-wing region and 3′-wing region comprises at least one nucleic acid analogue, and/or ii) a MixmeR of DNA and at least one nucleic acid analogue.

10. The synthetic antisense oligonucleotide of claim 9, wherein the at least one nucleic acid analogue comprises an LNA or a 2′-methoxyethyl (MOE)-modified nucleotide.

11. The synthetic antisense oligonucleotide of claim 10, wherein the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA.

12. (canceled)

13. The synthetic antisense oligonucleotide of claim 11, wherein the 5′-wing region comprises three, four, or five consecutive LNAs or 2′-methoxyethyl (MOE)-modified nucleotides.

14. (canceled)

15. The synthetic antisense oligonucleotide of claim 13, wherein the 3′-wing region comprises two, three or four, five consecutive LNAs or 2′-methoxyethyl (MOE)-modified nucleotides.

16. The synthetic antisense oligonucleotide of claim 15, wherein at least one internucleotide linkage of the synthetic antisense oligonucleotide is a phosphorothioate internucleotide linkage.

17. The synthetic antisense oligonucleotide of claim 16, wherein each internucleotide linkage of the synthetic antisense oligonucleotide is a phosphorothioate internucleotide linkage.

18. The synthetic antisense oligonucleotide of claim 1, wherein the synthetic antisense oligonucleotide comprises a nucleic acid sequence comprising at least 80% identical to a sequence selected from any one of SEQ ID NOs: 1-2566 and 2580-3987.

19. The synthetic antisense oligonucleotide of claim 1, wherein the synthetic antisense oligonucleotide comprises a nucleic acid sequence comprising at least 80% identical to a sequence selected from any one of SEQ ID NOs: 1-28, 31-37, 378, 385, 387, 2010-2018, and 2564-2566.

20. The synthetic antisense oligonucleotide of claim 1, wherein the synthetic antisense oligonucleotide comprises a nucleic acid sequence comprising at least 80% identical to a sequence selected from any one of SEQ ID NOs: 1-2013, 2564-2566, and 2580-2947.

21.-23. (canceled)

24. A method of modulating expression or activity of one or more pulmonary expressed lncRNAs in a subject, comprising administering an effective amount of the synthetic antisense oligonucleotide of claim 1 to the subject in need thereof, wherein the synthetic antisense oligonucleotide modulates expression or activity of one or more pulmonary expressed lncRNAs in the subject.

25.-26. (canceled)

27. The method of claim 24, wherein the synthetic antisense oligonucleotide reduces RNA expression level, protein expression level, or both, of one or more fibrotic genes selected from a group consisting of COL3A1, COL1A1, ACTA2, POSTN, FN1, FAP, CTHRC1, THBS1, and THBS2.

28.-36. (canceled)

37. A method of i) diagnosing idiopathic pulmonary fibrosis (IPF), or ii) monitoring, determining or predicting severity or progression of idiopathic pulmonary fibrosis (IPF) in a subject, the method comprising:
(a) obtaining a sample from the subject;
(b) determining an RNA expression level of one or more pulmonary expressed lncRNAs; and
(c) diagnosing idiopathic pulmonary fibrosis (IPF), or monitoring, determining or predicting severity or progression of idiopathic pulmonary fibrosis (IPF) in the subject based on the measurement from step (b).

38. (canceled)

39. The method of claim 37, wherein the one or more pulmonary expressed lncRNAs are transcribed from a region located within a genomic locus selected from a group consisting of chr21: 42009177-42055325 (strand +); chr15: 38864806-39427195 (strand −); chr15: 38871616-38880364 (strand −); chr6: 169171312-169188635 (strand −); chr4: 76758554-76801964 (strand −); chr17: 50199876-50215922 (strand +), chr2: 215717811-215721161 (strand +); chr14: 74552181-74560688 (strand +); chr2: 188966458-188984794 (strand −); chr11: 1218530-1220242 (strand +); chr6: 74069451-74690727 (strand +); chr16: 86845338-87058332 (strand −); and chr9: 22646200-22824213 (strand +), relative to human genome GRCh38.p14 (GCF_000001405.40).

40.-43. (canceled)

44. The method of claim 39, wherein the RNA expression level of the one or more pulmonary-expressed lncRNAs is at least about 2 folds higher in the pulmonary myofibroblasts than in the pulmonary fibroblasts.

45. The method of claim 37, wherein (b) further comprising determining an RNA expression level of a plurality of genes selected from a group consisting of COL3A1, COL1A1, ACTA2, POSTN, FN1, FAP, CTHRC1, THBS1, and THBS2.
